SEQUENCE LISTING (2 sequences)

SEQ ID NO: 1 - protein, 129 aa, Homo sapiens
SMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRR

SEQ ID NO: 2 - protein, 135 aa, Homo sapiens
MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQE

(19) United States
(12) Patent Application Publication
(10) Pub. No.: US 2004/0005689 A1
(43) Pub. Date: Jan. 8, 2004

(54) Crystal structure of the (AML1 Runt domain)/(CBFBETA) heterodimer and the ternary complex with DNA

(75) Inventors: Alan John Warren, Huntingdon (GB); Terence Rabbitts, Cambridge (GB)

Correspondence Address: PALMER & DODGE, LLP, KATHLEEN M. WILLIAMS, 111 HUNTINGTON AVENUE, BOSTON, MA, 02199, US

(73) Assignee: Medical Research Council

(21) Appl. No.: 10/253,084

(22) Filed: Sep. 24, 2002

Related U.S. Application Data
(63) Continuation of application No. PCT/GB01/01316, filed on Mar. 26, 2001.

(30) Foreign Application Priority Data
Mar. 24, 2000 (GB) 0007267.8
Feb. 23, 2001 (GB) 0104527.7

(51) Int. Cl. C12N 9/22; G06F 19/00; G01N 33/48; G01N 33/50

(52) U.S. Cl. 435/199; 702/19

(57) ABSTRACT

The present invention relates to a crystal structure for the AML1 Runt domain and/or CBFβ. In particular, the present invention relates to a crystal structure for an AML1 Runt domain:CBFβ, complex and a ternary AML1 Runt domain:CBFβ:DNA complex; and the use of the crystal structures to identify ligands capable of binding to the AML1 Runt domain and/or CBFβ, and/or capable of modulating the DNA-binding capacity of the AML1 Runt domain.

The present invention relates to a crystal structure for the AML1 Runt domain and/or CBFβ. In particular, the present invention relates to a crystal structure for an AML1 Runt domain:CBFβ complex and a ternary AML1 Runt domain:CBFβ:DNA complex; and the use of the crystal structures to identify ligands capable of binding to the AML1 Runt domain and/or CBFβ, and/or capable of modulating the DNA-binding capacity of the AML1 Runt domain.

### BACKGROUND

Leukaemias are characterised by the presence of recurrent chromosomal translocations (Rabbitts, 1994). The genes associated with these chromosomal breakpoints in acute leukaemias frequently encode transcription factors which have been demonstrated to play pivotal roles in normal development and leukaemogenesis. The core binding factors (CBFs) are one such family. These heterodimeric transcription factors consist of a DNA binding α subunit, and a non-DNA-binding β subunit, which associates well with the α subunit (Ogawa et al., 1993b). All α subunits share an evolutionarily conserved region of 128 amino acids known as the Runt domain (FIG. 2C), which mediates both DNA binding and heterodimerisation to the β-subunit. The Drosophila gene runt, which is the “founding member” of the α subunit family, is required for segmentation, sex determination and neurogenesis. Three mammalian genes encode the α-subunit: AML1/CBFA2/PEPBP2αB (herein called AML1) (Miyoshi et al., 1991), AML2/CBFA3/PEBP2αC (Levanon et al., 1994), and AML3/CBFA1/PEBP2αA/Osf2 (Ogawa et al., 1993b) (herein called AML3). Only one gene (CBFB) is known to encode the mammalian β subunit, called CBFβ (Ogawa et al., 1993a) (Wang et al., 1993), whereas two homologues brother, and big brother, have been identified in Drosophila (FIG. 2D) (Golling et al., 1996).

The AML1 gene was originally cloned from one of the most frequently acquired cytogenetic abnormalities in acute myeloid leukaemia (AML), the t(8;21)(q22;q22) chromosomal translocation (Miyoshi et al., 1993), and was subsequently shown to be involved in the recurrent chromosomal translocation t(12;21)(p13;q22) associated with childhood acute lymphoblastic leukaemias (Golub et al., 1994) (Romana et al., 1995), and the translocation t(3;21)(q26;q22) associated with therapy-related leukaemias and myelodysplasia (Nucifora et al., 1993). Nonsense, missense and frameshift mutations in the AML1 gene are associated with sporadic AML (Osato et al., 1999), and congenital mutations in AML1 have been described in individuals with the rare autosomal dominant disease, familial platelet disorder (FDP), in which there is a congenital predisposition to the development of AML (Song et al., 1999). The disease-associated mutations in the sporadic and congenital disorders cluster within the Runt domain of AML1, which spans residues 50-177 out of 451. The C terminus is rich in proline-serine-threonine, which is necessary for transcriptional activation of target genes. In man, mutations in AML3 are associated with cleidocranial dysplasia (CCD), an autosomal dominant disorder of skeletal morphogenesis (Mundlos et al., 1997) (Lee et al., 1997), and again, the majority of the point mutations associated with this disorder cluster within the Runt domain (Lee et al., 1997) (Quack et al., 1999) (Zhou et al., 1999). Thus, the conserved Runt domain of the CBF family of transcription factors is a key target for disease-associated mutations in man.

The gene encoding CBFβ, the β-subunit of the CBFs, is also involved in chromosomal translocations in AML as a result of the inv(16)(p13q22), t(16;16) and del(16)(q22), which fuse the N-terminal 165 amino acids of CBFβ in frame with a C-terminal portion of the smooth muscle myosin heavy chain in the M4Eo French-American-British (FAB) subtype of AML (15% of AML) (Liu et al., 1993). Together, the heterodimeric CBF transcription factor genes AML1 and CBFB are the most frequently mutated in human acute leukaemia, accounting for 25% of AML and 20% of paediatric common B cell acute lymphoblastic leukaemia (Look, 1997). Chromosomal translocations involving the AML1 and CBFB genes result in the production of novel chimaeric fusion proteins, all of which retain the core Runt domain which forms DNA binding complexes with CBFβ. Chromosomal translocations involving AML1 are thought to create dominant transcriptional repressor proteins, which may have a common mechanism of action in leukaemogenesis. Thus, the t(8;21) translocation fuses the ETO (MTG8) co-repressor C-terminal to the Runt domain, and inhibits transcription through recruitment of histone deacetylase (Gelmetti et al., 1998) (Lutterbach et al., 1998); the t(12;21) fuses a repressor domain from the TEL gene to the N-terminus of the AML1 protein to create a constitutive repressor (Hiebert et al., 1996). Recent data also demonstrates that the inv(16) fusion protein product recruits the co-repressor mSin3A (Lutterbach et al., 1999).

Gene targeting experiments in mice have revealed essential roles for the core binding factor proteins in normal osteogenesis and haematopoiesis. Aml3 is essential for osteoblast differentiation and bone development, as mice homozygous for a null mutation in Aml3 completely lack bone formation (Komori et al., 1997) (Otto et al., 1997). Null mutations in the genes encoding either murine Aml1 or Cbfβ result in failure of foetal liver-derived haematopoiesis (Okuda et al., 1996) (Wang et al., 1996b) (Wang et al., 1996a), as the complex is essential for the emergence of definitive haematopoietic progenitors in the developing embryo (North et al., 1999). In Drosophila, a mutant Runt protein which was specifically inactivated for heterodimerisation with Brother protein was defective in several in vivo functional assays (Li and Gergen, 1999). Thus, the function of the core binding family of transcription factors is critically dependent on the interaction of the Runt domain with CBFβ and its homologues.

AML1 binds as a monomer to the core DNA sequence TGT/cGGT, which is present in a number of different viral and cellular promoters and enhancers, as well as haematopoietic cell-specific genes including the T cell receptor α, -β, -γ, CD3ε, myeloperoxidase, neutrophil elastase, granzyme B and in several genes encoding cytokines and their receptors (Thornell et al., 1991) (Manley et al., 1993) (Giese et al., 1995) (Rodan and Harada, 1997). Methylation and ethylation interference experiments show that the Runt domain binds to the major groove of DNA (Thornell et al., 1988) (Melnikova et al., 1993). CBFβ enhances the DNA binding activity of AML1 by at least two distinct mechanisms. Electrophoretic mobility shift assays show that CBFβ enhances DNA binding of the full length AML1 molecule 40 fold by overcoming an inhibitory mechanism mediated by sequences C-terminal and N-terminal to the Runt domain (Kanno et al., 1998a). In vitro experiments have demonstrated that the Runt domain of AML1 binds to a high affinity recognition site with a dissociation constant of 50 nM (Crute et al., 1996). DNA binding of the isolated Runt domain is enhanced five to six fold by addition of CBFβ (Ogawa et al., 1993a), but the DNA footprint is not extended in ethylation interference analysis (Kamachi et al., 1990). Runt domain Cys 81 is protected from oxidation on binding CBFβ by a mechanism which does not depend on steric hindrance (Akamatsu et al., 1997a). From these data, it has been suggested that the Runt domain undergoes a conformational change on binding to CBFβ which results in enhanced DNA binding and alters the intrinsic oxidisability of Cys 81, but direct evidence for this has not yet been obtained.

The leukaemia-associated chromosomal translocations involving AML1 and CBFB genes result in fusion proteins, in which the AML1 Runt domain remains competent for heterodimerisation to CBFβ. This function is also predicted to be retained by many of the AML1 point mutants associated with sporadic AML and the FPD/AML pedigrees. As the active heterodimer is essential for normal haematopoiesis, heterodimerisation competency of mutant AML1 alleles may reduce the availability of the β-subunit to the active gene product, thus altering its transcriptional transactivation properties. Heterodimerisation may also underlie the process of leukaemic transformation in the M4Eo FAB subtype of AML, where the CBFB-MYH11 fusion gene product may sequester the functional AML1 subunit in an inactive transcription complex.

To date, no crystal structures have been provided for the AML1 Runt domain, or CBFβ, either alone or as a heterodimeric complex; moreover, a crystal structure of the AML1 Runt domain:CBFβ:DNA ternary complex, which would explain the interaction of the AML1 Runt domain:CBFβ complex with DNA, has not been available.

### SUMMARY OF THE INVENTION

The present invention provides a crystal comprising the AML1 Runt domain, and a crystal comprising CBFβ, as well as crystal structures of complexes thereof with each other and with DNA.

As mentioned above, AML1 and CBFβ dimerise in vivo to produce a transcription factor complex, and the Runt domain of AML1 is responsible for heterodimerisation to the β-subunit. In a further aspect, the present invention provides a crystal structure for a complex between the AML1 Runt domain and CBFβ. The structure of this crystal has been determined and is set forth in Table 3.

Elucidation of the crystal structure of the AML1 Runt domain:CBFβ complex reveals the regions of the AML1 Runt domain and CBFβ which are involved in heterodimerisation. In a further aspect, the present invention provides a crystal structure comprising the regions of the AML1 Runt domain and/or the regions of CBFβ which are involved in heterodimerisation.

In particular, the crystal structure indicates that the regions of the AML1 Runt domain which are involved in heterodimerisation include loops βF-G, βO-A, βB-C and βC-D and strands βC, βF and βG, and that the regions of CBFβ which are involved in heterodimerisation include arnino-terminal loop and helix H1; strand β1 and loop β1-H3; strands β2, β3 and the connecting loop β2-β3; and strand β4 and the at least part of loop β4-β5. The residues of the AML1 Runt domain and CBFβ involved in heterodimerisation are shown in Table 2.

The crystal structure of the heterodimeric complex enables a model to be produced for AML1, CBFβ and the heterodimerisation region. Thus, the present invention provides a model which can be used to screen for ligands which are capable of interacting with the AML1 Runt domain and/or CBFβ.

The present invention moreover provides a crystal structure of an AML1 Runt domain:CBFβ:DNA ternary complex. Such a structure may be used to produce a model of the AML1 Runt domain:CBFβ—DNA interaction, and accordingly of the key interactions between the polypeptides and DNA which are important in mediating DNA binding. Thus, the present invention provides a model which can be used to screen for ligands which are capable of modulating the DNA binding capacity of the AML-1 Runt domain. Such a ligand may, for example, induce or prevent a CBFβ-associated conformational change in AML1, modulate DNA-induced conformation change in the AML1 Runt domain:CBFβ heterodimer, or block the DNA binding site on the AML1 Runt domain.

The above model indicates that the regions of the AML1 Runt domain involved in DNA interaction include loops A′-B and E′-F and the C-terminus. The above model also provides information on the structural rearrangements of the Runt domain induced by the conformational change. In particular, the conformational change results in ordering of the C-terminal residues of the Runt domain and shifting of the βA′-βG′β sheet towards the βE′-F loop. There is also a shift in the position of Cys 81 of the Runt domain, such that it may form hydrogen-bonding interactions with Thr 169 and/or Val 137. From the model, it can be predicted that stabilisation of the Tyr162 side chain of the Runt domain induces the conformational change.

The present invention also provides a computer readable medium having stored thereon the crystal structure of the AML1 Runt domain and/or CBFβ, or one of the above-mentioned models.

In addition to the ligand itself, the present invention also provides a pharmaceutical composition comprising such a ligand, and a method of treating and/or preventing a disease comprising administering such a ligand or pharmaceutical composition to a mammalian patient. The method is predicted to be particularly useful in treating and/or preventing leukaemias which are associated with chromosomal translocations involving AML1 and CBFB genes.

### BRIEF DESCRIPTION OF DRAWINGS

FIG. 1 The crystal packing arrangement in the unit cell. Six Runt domain subunits and four CBFβ subunits are packed in alternating layers in the crystal. One layer is composed of two dimers of heterodimers (Runt domain/CBFβ)₂, and the second comprises a single Runt domain homodimer. Runt domain subunits are labelled A, C, E, G, Q and R; CBFβ subunits are labelled B, D, F and H.

FIG. 2 Structure of the Runt domain/CBFβ heterodimeric complex

(a) A stereoscopic diagram of the Cα trace of the AML1 Runt domain, residues 54-178, bound to CBFβ residues 2-135, prepared with MOLSCRIPT (Kraulis, 1991). CBFβ residues 73-78 are highly disordered and are not shown. The numbering corresponds to the amino acid sequences of human AML1 (Miyoshi et al., 1991) and human CBFβ (Liu et al., 1993).

(b) Ribbon diagram of a dimer of Runt domain/CBFβ heterodimers (two per asymmetric unit). The Runt domain β-strands are labelled βO to βG, consistent with the established immunoglobulin fold nomenclature (Nagata et al., 1999). The extensions to strands βA and βG, respectively, have been labelled βA′ and βG′. The β-strands of CBFβ are labelled β1-6, and the helices numbered H1-5, consistent with previous nomenclature (Goger et al., 1999). Only one hydrogen bond corresponding to a short potential 3₁₀ helix was seen in the region corresponding to helix H4, which was not therefore represented. The figure was prepared with MOLSCRIPT (Esnouf, 1997).

(c) Sequence alignment of the human AML1 Runt domain with human homologues AML1, AML2 and AML3, PEBP2αB from mouse, SpRunt from the sea-urchin Strongylocentrotus purpuratus, Xanml1 from Xenopus laevis, Lozenge and Runt from Drosophila, RNT-1 from C. elegans (program CLUSTALW (Thompson et al., 1994), manually adjusted). Absolutely conserved residues are shaded in dark grey, conservatively substituted residues are shaded in pale grey. Secondary structure elements follow the scheme of (a). The last line of the alignment shows the structural homology of the Runt domain to murine STAT3β. Conserved residues in the sequence are indicated. The numbering for STAT3β is based on the murine sequence (Acc. No. P42227).

(d) Sequence alignment of human CBFβ and the Drosophila homologues Brother and Big Brother. The numbering is for human CBFβ (Acc. No. Q13951). Secondary structure elements follows the scheme of (b). Residues 70-74 in CBFβ have no equivalent in Drosophila, and are indicated by dashed lines.

FIG. 3 The mode of interaction between the Runt domain and CBFβ.

(a) Runt domain (Runt) and CBFβ viewed parallel to the long axis of CBFβ. The concave surface of the Runt domain β-sheet, formed from strands βG, βF and βC, packs against the complementary concave strand β3 of CBFβ.

(b) Two views of the Runt domain/CBFβ structure related by a 180° rotation about the vertical axis. The long axes of the CBFβ and Runt domain β-barrels are orthogonal to one another.

FIG. 4 The relative orientations of interacting Runt domain subunits differs in the dimer of heterodimer and homodimer interfaces.

The figure was generated by least squares superposition of the Runt domain chains C and A onto the Runt domain chains Q and R. Chains A and C are represented by thin lines, and chain R is shown in bold. Chain Q, which was superimposed onto chain C, is not shown.

FIG. 5 The Runt domain complexed to CBFβ has a novel conformation.

Least squares superposition of the Cα trace of the Runt domain from the crystal structure onto the most closely related NMR conformer (PDB code 1cmo (Nagata et al., 1999)), (the analysis was performed for all the NMR conformers, and similar results were obtained). The NMR structure of the Runt domain is depicted as thick lines, and the crystal structure is depicted as thin lines.

FIG. 6 The location of human disease mutations on the core binding factor structure

(a) A ribbon diagram of the Runt domain/CBFβ heterodimer is shown in which residues mutated in cleidocranial dysplasia (G158, M124, T149, L148, F146, S140, R139, R174 and K167), familial platelet disorder (FPD) and acute myeloid leukaemia (R135, C81, C83, R80, D171, R139, R174 and R177) are indicated.

(b) Model orienting the core binding factor heterodimer with respect to B-form DNA. The model was generated by least squares superposition of the coordinates of the STAT3β-DNA complex (PDB code, 1bgl (Becker et al., 1998)) onto the AMT1 Runt domain. The amino acid residues mutated in human disease mutations are indicated as spheres.

FIG. 7 Overall structure of the AML1 Runt domain-CBFβ-DNA ternary complex.

(a) Ribbon diagram of the ternary complex. This view has the DNA axis parallel to the plane of the page. The Runt domain is shown in cyan; CBFβ, magenta; DNA strand E, yellow; DNA strand F, mauve. Strands and helices are labelled consistent with (Warren et al., 2000), except for an additional β-strand in CBFβ, labelled β3′. The structural features of the Runt domain that contact the DNA are highlighted: strand βA′ and part of loop βA′-B (residues T77-T84) in red; loop βE-F (residues N132-S145) in green, including the ‘wing’ (residues R139-S145); strand βG′ (residues 166-169) in grey; the extended C-terminal ‘tail’ (residues V170-R177) in blue. Residues 75-79 of CBFβ are disordered and are shown as a dashed line. Numbering corresponds to the amino acid sequences of human AML1 (Miyoshi et al., 1991) and CBFβ (Liu et al., 1993). Figures were prepared with the program PREPI.

(b) View of the ternary complex rotated by 90° about the x-axis so that the DNA axis is perpendicular to the page.

(c) Surface representation of the ternary complex. The Runt domain clamps DNA strand F between the C-terminal ‘tail’ in the major groove and the ‘wing’, which straddles across the nucleotide backbone into the minor groove. The figure was prepared using GRASP (Nicholls et al., 1991).

(d) The sequence of the DNA duplex used for the co-crystallisation is shown with the numbering scheme used in the text.

FIG. 8 DNA recognition by the AML1 Runt domain-CBFβ protein complex

(a) Polar (red) and hydrophobic (blue) interactions between the DNA and the protein. DNA strand E is shown in yellow, strand F, mauve. The central 6 base pairs corresponding to the consensus DNA binding sequence are highlighted in bold. The indirect water contact from T84 to base T3, atom 02P (strand E) is found in only one of the two complexes.

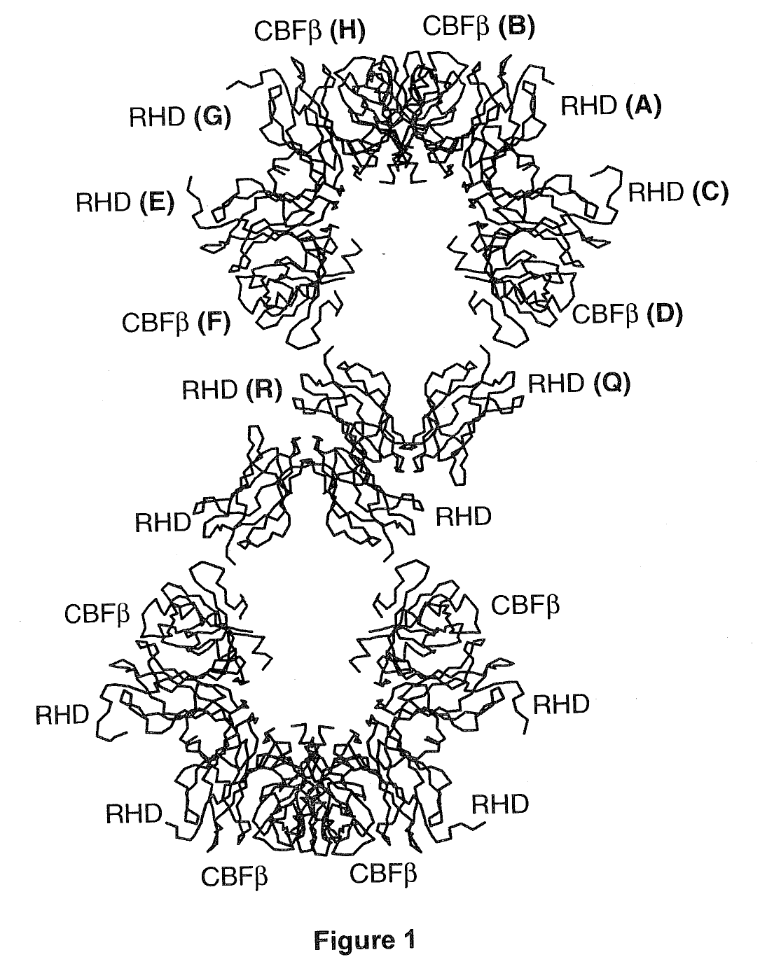

(b) Ribbon diagram of the interactions of the AML1 Runt domain with DNA. Structural elements involved in DNA binding are highlighted as in FIG. 1. Residues making contacts to DNA that are mutated in human disease are indicated by black boxes. Polar interactions are indicated by dashed lines. The included water molecules (red spheres) are present in both copies of the complex. Figure was prepared with MOLSCRIPT (Kraulis, 1991).

FIG. 9 DNA-induced conformational change in the Runt domain-CBFβ complex.

Least squares superposition of the Cα traces of the Runt domain-CBFβ crystal structure (pdb code 1 e50) onto the Runt domain-CBFβ-DNA ternary complex crystal structure. In the ternary complex, the Runt domain is coloured cyan, and CBFβ is magenta; the superimposed binary complex shows the Runt domain in brown, CBFβ in yellow. Arrows indicate Runt domain residue G143 and the C-terminal ‘tail’. Residues K144 (‘wing’), D110 and E111 (loop βC-D) are indicated. The two structures differ in the relative positions of the βE′-F loop and the C-terminal ‘tail’.

FIG. 10 β-galactosidase activity conferred by wild type and mutated Runt domains in yeast one-hybrid assays

(a) Filter β-galactosidase assays. The GAL4-Runt domain fusion derivatives carried in the yeast transformants are indicated. Six independent colonies were streaked in each segment.

(b) Quantitative liquid β-galactosidase assays.

FIG. 11 Comparison of DNA recognition mechanism of the AML1 Runt domain, class II Rel proteins and NFAT.

Specific base contacts are indicated by arrows for the human AML1 Runt domain (this work), mouse p65 (Chen et al., 2000), Gambifl (Cramer et al., 1999) and NFAT (Chen et al., 1998). The buttressing hydrogen bonds made from the conserved glutamates or aspartate (Runt domain) are indicated (dashed lines). The conserved GG core elements are highlighted in red.

### DETAILED DESCRIPTION OF THE INVENTION

In one aspect of the present invention, there is provided a crystal comprising the AML1 Runt domain and/or CBFβ and/or the DNA bound by the AML1:CBFβ dimer.

As used herein, the term “crystal” means a structure (such as a three dimensional (3D) solid aggregate) in which the plane faces intersect at definite angles and in which there is a regular structure (such as internal structure) of the constituent chemical species. Thus, the term “crystal” can include any one of: a solid physical crystal form such as an experimentally prepared crystal, a 3D model based on the crystal structure, a representation thereof such as a schematic representation thereof or a diagrammatic representation thereof, a data set thereof for a computer.

As explained above, the Runt domain is highly conserved between members of the CBF family of transcription factors, and the majority of disease-associated mutations cluster within this domain. The Runt domain spans an 128 amino acid region, and mediates both DNA binding and heterodimerisation to the β-subunit. The AML1 Runt domain spans amino acid residues 50-177 out of 451. The complete coding sequence for AML1 (including the three versions generated by alternative splicing) are available (for example, GenBank accession numbers NM001754, AF 0258411, E05037 and D43967-D43969).

CBFβ is the β-subunit of the core-binding factors, which associates with any of the three α-subunits (AML1, AML2 and AML3). The complete sequence of CBFβ is known (see, for example, Ogawa et al (1993) Virology 194:314-331, or GenBank accession No: L20298).

In another aspect of the present invention, there is provided a crystal comprising a complex between the AML1 Runt domain and CBFβ.

The core binding factors function as heterodimers of a DNA-binding α-subunit (such as AML1) and a non-DNA-binding β-subunit (CBFβ). The crystals of this aspect of the invention may be made by crystallising pre-formed heterodimers, so as to co-crystallise the α and β-subunits.

In a preferred embodiment of this aspect of the invention, the asymmetric unit of the crystal comprises six Runt domain subunits and four CBFβ subunits arranged as two dimers of Runt domain/CBFβ heterodimers (Runt domain/CBFβ)₂ and a Runt domain homodimer (as shown in FIG. 1). Most preferred is a crystal with has P2₁ symmetry which comprises a unit cell having the following dimensions: a=103.2 Å, b=79.4 Å, c=130.1 Å. Depending on the particular conditions used for crystallisation, the above parameters characterising the unit cell may vary within a limited range, for example, a, b and c may vary by up to 5.

In a highly preferred embodiment of this aspect of the invention, the crystal has the structural coordinates as shown in Table 3.

As used herein, the term “structural co-ordinates” refer to a set of values which define the position of one or more amino acid residues with reference to a system of axes.

The present invention also provides a crystal structure of the AML1 Runt domain and/or CBFβ. The structure of the crystal may be solved by any known method, for example by X-ray diffraction followed by multiple anomalous dispersion (MAD).

The crystal structure of a AML1 Runt domain:CBFβ complex of the present invention provides information about the overall structure of each subunit and about the particular elements of secondary structure for each subunit which are involved in heterodimerisation. In this respect, the crystal structure indicates that the regions of which AML1 are important in heterodimerisation include various loops between β strands (namely βF-G, βO-A, βB-C and βC-D) and a number of β-strands (βC, βF and βG). As regards CBFβ, the following elements of secondary structure are indicated as being of importance: amino-terminal loop and α-helix H1; strand β1 and the loop between β1 and α-helix H3; β-strands β2, β3 and the connecting loop β2-β3; and β-strand β4 and the proximal part of loop β4-β5.

As used herein, the term “α-helix” means a helical or spiral configuration of a polypeptide chain in which successive turns of the helix are held together by hydrogen bonds between the amide (peptide) links, the carbonyl group of any given residue being hydrogen-bonded to the imino group of the third residue behind it in the chain. This is the case for all of the carbonyl and amide groups of the peptide bonds of the main chain. Typically, the α-helix has 3.6 residues per turn and the translation or pitch along the helical axis is 1.5 Å per residue and 5.4 Å per turn. The helix may be left- or right-handed, the latter being much more common. The a-helix is one of the two basic elements of the secondary structure adopted by the polypeptide chain within the hydrophobic core of a globular protein. The other basic element is the β strand.

β strands are usually from 5 to 10 residues long and are in an almost fully extended conformation with φ, ψ angles within the broad structurally allowed region in the upper left quadrant of the Ramachandran plot. These β strands are aligned adjacent to each other such that hydrogen bonds can form between C'O groups of one β strand and NH groups on an adjacent β strand and vice versa. Parallel or antiparallel β sheets may be formed from several such β strands which are “pleated” with C_{α} atoms successively a little above and below the plane of the β sheet. The side chains follow this pattern such that within a β strand they also point alternatively above and below the β sheet.

The amino acid residues involved in heterodimerisation (as elucidated from the crystal structure) are shown in Table 2.

By using known modelling techniques, the crystal structure of the present invention can be used to produce a model for at least part of the AML1 Runt domain and/or at least part of CBFβ and/or for DNA binding by the AML1 Runt domain.

As used herein, the term “modelling” includes the quantitative and qualitative analysis of molecular structure and/or finction based on atomic structural information and interaction models. The term “modelling” includes conventional numeric-based molecular dynamic and energy minimisation models, interactive computer graphic models, modified molecular mechanics models, distance geometry and other structure-based constraint models.

The crystal structure of the present invention can be used to generate a structural model such as a three dimensional (3D) structural model (or a representation thereof). Alternatively, the crystal structure may be used to generate a computer model for the structure.

In a preferred embodiment, a model for DNA binding by the AML1 Runt domain is generated by least squares superimposition of the coordinates of the STAT3β/DNA co-crystal complex on to the AML1 Runt domain. STAT3β is a transcription factor to which the AML1 Runt domain is closely related.

In a further aspect, the invention provides a crystal structure for a AML1:CBFβ:DNA ternary complex. In a preferred aspect, the crystal has the coordinates set forth in table 4.

The structure may be used to evaluate the accuracy of models prepared as described above; moreover, more accurate representations of the ternary complex may be based directly on the coordinates, thus obviating the need for models as described above altogether.

In another aspect of the present invention, the above-mentioned models and structures may be used to screen for ligands which are capable of binding the AML1 Runt domain and/or CBFβ and/or which are capable of modulating the DNA binding capacity of the AML1 Runt domain.

The screen may employ a solid 3D screening system or a computational screening system. Using these systems, test compounds may be screened to find those which interact spatially and preferentially with the AML1 Runt domain and/or CBFβ and/or DNA, through either computational or manual docking.

As used herein, the term “test compound” means a compound which may be tested for its capacity to interact with the AML1 Runt domain and/or CBFβ and/or to modulate the DNA binding capacity of the AML1 Runt domain. The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a structural or functional mimetic, a peptide, a peptidomimetics, a derivatised test compound, a peptide cleaved from a whole protein, or one or more peptides synthesised synthetically (such as, by way of example, either using a peptide synthesiser or by recombinant techniques or combinations thereof, a recombinant test compound, a natural or a non-natural test compound, a fusion protein or equivalent thereof and mutants, derivatives or combinations thereof.

Thus, the structural information from the crystal structure of the present invention is useful in the design of potential ligands capable of interacting with the AML1 Runt domain and/or CBFβ and/or capable of modulating the DNA binding capacity of the AML1 Runt domain, and the models of the present invention are useful to examine the effect such a ligand is likely to have on the structure and/or function of the AML1 Runt domain and/or CBFβ. The present invention also relates to a ligand identified using such methods.

As used herein, the term “ligand” refers to a test compound capable of binding to AML1 Runt domain and/or CBFβ. Preferably the ligand modulates the interaction between AML1 Runt domain and CBFβ, and/or modulates the DNA binding capacity of the AML1 Runt domain.

The ligands may be natural or synthetic. The term “ligand” also refers to a chemically modified ligand.

In one aspect, the identified ligand may act as a ligand model (for example, a template) for the development of other compounds.

Ligands which interact with the heterodimerisation regions of AML1 Runt domain or CBFβ may influence the heterodimerisation capacity. Such ligands may interact with at least some of loops βF-G, βO-A, βB-C and βC-D and strands βC, βF and βG of the AML1 Runt domain or at least some of the following elements of secondary structure of CBFβ: amino-terminal loop and helix H1; strand β1 and loop β1-H3; strands β2, β3 and the connecting loop β2-β3; and strand β4 and the at least part of loop β4-β5. Such ligands may interact with the at least some of the heterodimerisation residues shown in Table 2, for example, a sufficient number of heterodimerisation residues to influence the heterodimerisation capacity. Further sites for ligand interaction will be apparent from the following examples.

As explained above, the fusion proteins resulting from leukaemia-associated chromosomal translocations involving AML1 and CBFB genes retain the capacity to heterodimerise. From a therapeutic perspective, it is therefore preferable for a ligand which binds to the heterodimerisation region of the active heterodimer also to be able to bind to the mutant heterodimer.

Ligands which are capable of modulating the DNA-binding capacity of the AML1 Runt domain may be able to bind the A′-B and E′-F loops and the C-terminus of the AML1 Runt domain.

As explained above, the binding of CBFβ enhances DNA binding by the Runt domain. The crystal structure of the present invention supports the theory that this is due to a conformational change in the Runt domain, and gives information about the structural rearrangements in the AML1 domain responsible for the increased binding affinity. The ligand of the present invention may enhance the DNA binding affinity of an AML1 Runt domain by inducing a conformational change in the Runt domain.

The conformational change may, for example, be analogous to the conformational change induced by CBFβ-binding, such as to result in ordering of the C-terminal residues of the Runt domain and shifting of the βA′-βG′β sheet towards the βE′-F loop and/or to cause a shift in the position of Cys 81 of the Runt domain, such that it may form hydrogen-bonding interactions with Thr 169 and/or Val 137.

Again, in a manner analogous to CBFβ binding, the ligand may stabilise the Tyr162 side chain of the Runt domain, thus inducing the conformational change.

Test compounds and ligands which are identified with the crystal of the present invention can be screened in assays such as are well known in the art. Screening can be, for example in vitro, in cell culture, and/or in vivo. Biological screening assays preferably centre on activity-based response models, binding assays (which measure how well a compound binds to the AML1 Runt domain and/or CBFβ), and bacterial, yeast and animal cell lines (which measure the biological effect of a compound in a cell). The assays can be automated for high capacity-high throughput screening (HTS) in which large numbers of compounds can be tested to identify compounds with the desired activity. The biological assay, may also be an assay for ligand binding activity a compound that selectively binds to AML1 Runt domain and/or CBFβ.

The ligand of the present invention may be used in a pharmaceutical composition, optionally together with a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the pharmaceutical composition is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The invention further provides a method of preventing and/or treating disease in a mammal, the method comprising administering to a mammal a ligand or pharmaceutical composition of the present invention.

Typically, a physician will determine the actual dosage which will be most suitable for an individual subject and it will vary with the age, weight and response of the particular patient and severity of the condition. The dosages below are exemplary of the average case. There can, of course, be individual instances where higher or lower dosage ranges are merited.

The compositions (or component parts thereof) of the present invention may be administered orally. In addition or in the alternative the compositions (or component parts thereof) of the present invention may be administered by direct injection. In addition or in the alternative the compositions (or component parts thereof) of the present invention may be administered topically. In addition or in the alternative the compositions (or component parts thereof) of the present invention may be administered by inhalation. In addition or in the alternative the compositions (or component parts thereof) of the present invention may also be administered by one or more of: parenteral, mucosal, intramuscular, intravenous, subcutaneous, intraocular or transdermal administration means, and are formulated for such administration.

By way of further example, the pharmaceutical composition of the present invention may be administered in accordance with a regimen of 1 to 10 times per day, such as once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The term “administered” also includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Hence, the pharmaceutical composition of the present invention may be administered by one or more of the following routes: oral administration, injection (such as direct injection), topical, inhalation, parenteral administration, mucosal administration, intramuscular administration, intravenous administration, subcutaneous administration, intraocular administration or transdermal administration.

The method of the present invention is particularly suited to the treatment or prevention of acute myeloid leukaemia (AML), childhood acute lymphoblastic leukaemias, therapy-related leukaemias, myelodysplasia, familial platelet disorder (FDP), cleidocranial dysplasia (CCD), and conditions associated with impaired osteogenesis and/or haematopoiesis.

In particular, the method of the invention is used to treat to prevent a leukaemia associated with chromosomal translocations involving the AML1 and/or the CBFB gene.

Production of the Crystal

The crystals of the present invention may be prepared by expressing a nucleotide sequence encoding the AML1 Runt domain and/or CBFβ, by the use of a suitable host cell, and then crystallising the purified protein(s).

As used herein, the term “nucleotide sequence” refers to nucleotide sequences, oligonucleotide sequences, polynucleotide sequences and variants, homologues, fragments and derivatives thereof (such as portions thereof) which comprise the nucleotide sequences encoding the AML1 Runt domain and/or CBFβ amino acid sequences. The nucleotide sequence may be DNA or RNA of genomic or synthetic or recombinant origin which may be double-stranded or single-stranded whether representing the sense or antisense strand or combinations thereof. Preferably, the term nucleotide sequence is prepared by use of recombinant DNA techniques (e.g. recombinant DNA). The nucleotide sequence may include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vitro activity or life span of nucleotide sequences of the invention.

To produce an AML1 Runt domain:CBFβ complex, it is preferable for the gene encoding the AML1 Runt domain and the gene encoding CBFβ to be “coexpressed” (i.e. driven from the same promoter).

The nucleotide sequence may encode the wild type AML1 runt domain amino acid sequence shown in SEQ ID No 1, and/or at least residues 2-135 of the wild type CBFβ sequence shown SEQ ID No 2, or a mutant or homologue thereof.

The term “wild type” refers to the phenotype that is characteristic of most of the members of a species occurring naturally and which contrasts with the phenotype of a mutant species.

The term “mutant” refers to any organism that has undergone mutation or that carries a mutant gene that is expressed in the phenotype of that organism. A mutation may arise due to a substitution of one nucleotide for another or from a deletion of a nucleotide or an insertion of a nucleotide relative to a referenced wild type sequence. These single nucleotide variations are sometimes referred to as single nucleotide polymorphisms (SNPs). Some SNPs may occur in protein-coding sequences, in which case, one of the polymorphic forms may give rise to the expression of a defective or other variant protein and, potentially, a genetic disease. Other SNPs may occur in noncoding regions. Some of these polymorphisms may also result in defective protein expression (e.g., as a result of defective splicing). Other SNPs may have no phenotypic effects.

As used herein, the term “mutant” refers to polypeptide comprising any one or more changes in the sequence (the structural co-ordinates) and of the amino acid residues in the AML1 Runt domain and/or CBFβ. In particular, but not exclusively, the mutation in the AML1 Runt domain and/or CBFβ may be in those regions involved in heterodimerisation (as shown in Table 2).

As used herein, the term “homologue” refers to an AML1 Runt domain and/or CBFβ or a portion thereof which may have deletions, insertions or substitutions of amino acid residues as long as the binding specificity of the molecule is retained. In this regard, deliberate amino acid substitutions may be made on the basis of similarity in polarity, charge, solubility, hydrophobicity, hydrophilicity, and/or the amphipathic nature of the residues as long as the binding specificity of the AML1 Runt domain and/or CBFβ molecule is retained.

A wide variety of host cells can be employed for expression of the nucleotide sequences encoding the AML1 Runt domain and/or CBFβ proteins of the present invention. These cells may be both prokaryotic and eukaryotic host cells. Suitable host cells include bacteria such as E. coli, yeast, filamentous fungi, insect cells, mammalian cells, typically immortalised, e.g., mouse, CHO, human and monkey cell lines and derivatives thereof. Preferred host cells are able to process the expression products to produce an appropriate mature polypeptide(s). Processing includes but is not limited to glycosylation, ubiquitination, disulphide bond formation and general post-translational modification.

The proteins comprising the AML1 Runt domain and/or CBFβ, produced by a host recombinant cell, may be secreted or may be contained intracellularly depending on the nucleotide sequence and/or the vector used. As will be understood by those of skill in the art, expression vectors containing the AML1 Runt domain and/or CBFβ encoding nucleotide sequences can be designed with signal sequences which direct secretion of the AML1 Runt domain and/or CBFβ coding sequences through a particular prokaryotic or eukaryotic cell membrane.

Other recombinant constructions may join the AML1 Runt domain and/or CBFβ encoding sequence to nucleotide sequence encoding a polypeptide domain which will facilitate purification of soluble proteins (Kroll D J et al (1993) DNA Cell Biol 12:441-53). Such purification facilitating domains include, but are not limited to, metal chelating peptides such as histidine-tryptophan modules that allow purification on immobilised metals (Porath J (1992) Protein Expr Purif 3 -.26328 1), protein A domains that allow purification on immobilised immunoglobulin, and the domain utilised in the FLAGS extension/affinity purification system (Immunex Corp, Seattle, Wash.). The inclusion of a cleavable linker sequence such as Factor XA or enterokinase (Invitrogen, San Diego, Calif.) between the purification domain and the AR and PR is useful to facilitate purification.

The AML1 Runt domain and/or CBFβ may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences.

Preferably the fusion protein will not hinder the heterodimerisation capacity of the AML1 Runt domain and/or CBFβ.

After expression, the proteins may be purified and/or concentrated, for example by immobilised metal affinity chromatography, ion-exchange chromatography, and/or gel filtration. For crystals of the AML1 Runt domain:CBFβ complex, the AML1 Runt domain and CBFβ fragments may be purified and allowed to dimerise in solution. The capacity of the complex to bind DNA may be determined by, for example, electrophoretic mobility shift assays.

The protein(s) may be crystallised using any of the known techniques. Usually, in a crystallisation process, a crystallisation buffer is prepared with a lower concentration of a precipitating agent necessary for crystal formation. For crystal formation, the concentration of the precipitating agent has to be increased, by addition of precipitating agent or by diffusion of the precipitating agent between the crystallisation buffer and a reservoir buffer. Diffusion may be achieved by known techniques such as the “hanging drop” or the “sitting drop” method. In these methods, a drop of crystallisation buffer containing the protein(s) is hanging above or sitting beside a much larger pool of reservoir buffer. Alternatively, the balancing of the precipitating agent can be achieved through a semi-permeable membrane that separates the crystallisation buffer and prevents dilution of the protein into the reservoir buffer.

Generating the Crystal Structure

Once the crystals have been obtained, the structure may be solved by known X-ray diffraction techniques. Many techniques use chemically modified crystals, such as those modified by heavy atom derivatization. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location(s) of the bound heavy metal atom(s) can then be determined by X-ray diffraction analysis of the soaked crystal. The patterns obtained on diffraction of a monochromatic beam of X-rays by the atoms (scattering centres) of the crystal can be solved by mathematical equations to give mathematical coordinates. The diffraction data are used to calculate an electron density map of the repeating unit of the crystal. The electron density maps are used to establish the positions of the individual atoms within the unit cell of the crystal. Blundel, T. L. and N. L. Johnson, Protein Crystallography, Academic Press (1976).

Those of skill in the art would understand that a set of structure coordinates determined by X-ray crystallography is not without standard error. For the purpose of this invention, any set of structure coordinates for AML1: CBFβ complexes that have a root mean square deviation of protein backbone atoms of less than 0.75 Å when superimposed (using backbone atoms) on the structure coordinates listed in Table 3 shall be considered identical.

Modelling

The three dimensional structure of a new crystal may be modelled using molecular replacement. The term “molecular replacement” refers to a method that involves generating a preliminary model of a molecule or complex whose structure coordinates are unknown, by orienting and positioning a molecule whose structure coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972).

Other molecular modelling techniques may also be employed in accordance with this invention. See, e.g., Cohen, N. C. et al., “Molecular Modelling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Using the structure coordinates of the AML1:CBFβ crystal complex provided by this invention, molecular replacement may be used to determine the structure coordinates of a crystalline mutant or homologue of AML1 and/or CBFβ or of a related protein (such as AML2, AML3, or another transcription factor).

Ligand Screening

One skilled in the art may use one of several methods to test compounds for their ability to associate with AML1 and/or CBFβ. This process may begin by visual inspection of, for example, a target site on the computer screen based on the structure coordinates given in Table 3. Selected test compounds may then be positioned in a variety of orientations, or docked, within an individual target site of AML1 and/or CBFβ as defined supra. Docking may be accomplished using software such as Quanta and Sybyl, followed by energy minimisation and molecular dynamics with standard molecular mechanics forcefields, such as CHARMM and AMBER. Specialised computer programs may also assist in the process of selecting potential ligands. These include:

1. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (Miranker, A. and M. Karplus, “Functionality Maps of Binding Sites: A Multiple Copy Simultaneous Search Method.” Proteins: Structure. Function and Genetics, 11, pp. 29-34 (1991)). MCSS is available from Molecular Simulations, Burlington, Mass.

3. AUTODOCK (Goodsell, D. S. and A. J. Olsen, “Automated Docking of Substrates to Proteins by Simulated Annealing”, Proteins: Structure. Function, and Genetics, 8, pp. 195-202 (1990)). AUTODOCK is available from Scripps Research Institute, La Jolla, Calif.

4. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

Once a ligand has been optimally selected or designed, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analysed for efficiency of fit to AML1 and/or CBFβ by the same computer methods described above.

### EXAMPLES

The following examples serve to illustrate the present invention, but should not be construed as a limitation thereof. The invention particularly relates to the specific embodiments described in these examples.

### Example 1 Crystallisation of the Runt Domain/CBFβ Complex

Protein Expression and Purification

The DNA sequence encoding human AML1, residues 50-183 is amplified from plasmid pET/RM7 (gift of Dr. F. Calabi) by the polymerase chain reaction. A DNA fragment is obtained with unique restriction sites for BamH1 and EcoR1. This PCR fragment is cloned into the mini-pRSET vector (gift from O. Perisic), which allows transcription from the T7 promoter and adds 16 residues at the N-terminus (MRGSHHHHHHGLVPRG). The DNA sequence encoding human CBFP residues 1-135, is amplified by PCR from cDNA synthesised from RNA purified from HEL cells, to give a fragment with unique restriction sites for Nde1 and BamH1. This fragment is cloned downstream of a Shine-Delgarno sequence in pBluescript, and subsequently subcloned into the mini-pRSET vector downstream of the AML1 coding sequence. This bicistronic clone allows coexpression of AML1 and CBFβ from the same T7 promoter. Coexpression of the human AML1 and CBFβ proteins is convenient for obtaining a soluble functional heterodimeric complex without a requirement for mutagenesis or refolding.

The pmini-α/β expression plasmid is transfected into E.coli C41 (DE3) cells (Miroux and Walker, 1996). Transformed cells are grown at 37° C. in 2×TY medium containing ampicillin at 100 μg/ml to an OD₆₀₀ of 0.6, induced for 4 hours with 1 mM isopropylthio-β-D-galactoside (IPTG). Cells are harvested and sonicated in 20 mM β-mercapto-ethanol, HEPES 20 mM pH 8.0, glycerol 10%, NaCl 500 mM, imidazole 20 mM. The expressed fragments of human AML1 (residues 50-183), herein referred to as the Runt domain, and CBFβ (residues 2-135) form a stable complex in solution which is fully active in sequence-specific DNA binding as determined by electrophoretic mobility shift assays (data not shown). The heterodimeric complex is purified from the lysate by immobilised metal affinity chromatography, ion-exchange chromatography, and gel filtration. To this end, the lysate is loaded onto a Ni2+-NTA column (Quiagen), washed extensively in the same buffer, and eluted (in 20 mM β-mercapto-ethanol, HEPES 20 mM pH 7.0, NaCl 500 mM, glycerol 10%, imidazole 150 mM) directly into tubes contained DTT and EDTA to final concentrations of 5 mM and 1 mM, respectively. The concentration of NaCl is reduced to 200 mM by dilution with NaCl-free buffer, and the sample applied to a Resource S column (Pharmacia) equilibrated in HEPES 20 mM pH 7.0, NaCl 200 mM, EDTA 1 mM and DTT, 5 mM. The eluate is applied to a HiPrep Sephacryl S100 column (Pharmacia), equilibrated in Hepes 20 mM, pH 6.6, NaCl 300 mM, DTT 1 mM and EDTA 0.1 mM. Pooled fractions are concentrated using a Centricon ultrafiltration membrane and aliquots stored in liquid nitrogen at a concentration of 12 mg/ml in a solution containing 20 mM Hepes pH 6.6, NaCl 300 mM, DTT 10 mM and EDTA 1 mM.

Preparation of Selenomethionine-substituted AML1/CBFβ

Selenomethionine (SeMet)-substituted protein is expressed from the pmini-α/β plasmid in the E.coli DE3 host, which is a methionine-requiring auxotroph. Log phase cells (OD₆₀₀=0.3) precultured in 2×TY, are diluted 1:100 into 2×M9 minimal medium supplemented with 0.4% glucose, 19 amino acids at 40 μg/ml, seleno-L-methionine (Sigma) at 40 μg/ml, and vitamins at 1 μg/ml ((Ramakrishnan and Biou, 1997)). Bacteria are left to grow at 37° C., and are induced after 8 hours (OD₆₀₀=0.6). Induction is at 25° C. overnight (OD₆₀₀=1.1). The method of protein purification is identical to that of the unsubstituted protein. The masses of the unsubstituted and SeMet-substituted proteins are assayed by MALDI mass spectrophotometry (CBFμ-observed 15786 Da, calculated 15923 Da; AML1-observed 16730 Da, calculated 16720 Da; SeMet CBFβ-observed 15880 Da, SeMet AML1-observed 16916 Da) and is consistent with complete substitution of methionine residues with SeMet, but loss of the N-terminal methionine of CBFβ.

Crystallisation

Initially, crystals are grown by the hanging drop vapour diffusion technique. The purified complex is soluble up to concentrations of 30 mg/ml. Crystals of the complex are grown as hanging drops against a reservoir containing 100 mM imidazole, 6% PEG 8000, 5% sucrose and 5 mM DTT. Droplets are initially set up at 18° C. using 1μl of 12 mg/ml protein solution and 1 μl of crystallisation solution. Trays are equilibrated at 21° C. for at least one week. To overcome problems with crystal twinning, macroseeding is used to obtain diffraction quality crystals. Seeds are washed in 100 mM imidazole, 5% sucrose, 5% PEG 8000, 200 mM NaCl, 5% 2-methyl-2,4pentanediol (MPD) (Hampton), transferred individually to fresh drops of 2 μl 12 mg/ml protein and 2 μl wash buffer which had been pre-equilibrated overnight against buffer without the MPD additive, supplemented with 1 mM DTT. Crystals grew to their final size over 2 weeks. They are harvested by gradual transfer to a final cryobuffer containing 30% sucrose, 12% PEG 8000, 5% MPD, 200 mM NaCl, 100 mM imidazole pH 6.5, for 30 seconds and then cryocooled in liquid nitrogen. Crystals are prepared from SeMet substituted protein under similar conditions, except that 5% MPD is incorporated as an additive in the initial drops. Crystals belonging to space group P61 are also obtained using a crystallisation solution with 400 mM NaCl, HEPES 50 mM pH 7.5, 5 mM DTT, 5% PEG 8000. These are cryocooled by prior transfer to a buffer containing 30% glycerol, 100 mM HEPES pH 7.5, 200 mM NaCl, 5% PEG 8000.

### Example 2 Crystal Structure Determination

Data Collection Crystals used for native protein data collection are typically 400×200×40 μm or larger, have P21 symmetry with unit cell dimensions of a=103.2 Å, b=79.4 Å, c=130.1 Å, β=101.63°. These crystals diffract to 2.6 Å. Se-Met substituted crystals have unit cell dimensions a=104.0 Å, b=79.3 Å, c=130.9 Å, β=101.5°, are somewhat smaller and diffract to only 2.8 Å resolution. Diffraction data for the native crystal are collected at ESRF beamline ID14-3 using a MAR CCD detector and the data for the Se-Met substituted crystal are collected at ID14-4 using an ADSC CCD detector. Native data are processed using MOSFLM (Leslie, 1992). Each data set is collected as 180 1° oscillations. For the Se-Met-substituted crystal, data sets are collected at two wavelengths, λ1=0.9793 Å and λ2=0.9795 Å, corresponding to the peak and inflection points in the fluorescence spectrum of a Se-Met-substituted crystal.

Data Processing

Data sets for the Se-Met-substituted crystal are integrated with HKL2000 and scaled with SCALEPACK. The native data are scaled and merged with SCALA. Final merging of the Se-Met data is carried out with SOLVE (Terwilliger and Berendzen, 1999). The R factor for dispersive differences between data collected at λ1 and data collected at λ2 is 0.046. The R factor for anomalous differences is 0.077 for λ1 and 0.066 for λ2. SOLVE is used to locate 21 Se sites. The SOLVE Z-scores for the Patterson correlation, cross-validation Fourier, native Fourier and mean figure of merit for the 21-site solution are 7.4, 52, 21 and 9.4, respectively. The overall Z-score is 66. The mean figure of merit is 0.47. Se-Met data to 3.5 Å resolution are used to provide an initial estimate for the phases of the native data set. Density modification is carried out to improve the phases and extend them to 2.6 Å resolution (Brünger et al., 1998). A model is built into the 2.6 Å resolution electron density maps using the program O (Jones et al., 1991), and refined using CNS (Brünger et al., 1998). The average B-factor for all atoms is 55.9 Å². The structure shows good stereochemistry with no residues in disallowed regions of the Ramachandran plot. Additional native data sets may be collected from beamline 9.6 (Daresbury UK), beamline X16 (Hamburg Germany), and ELETTRA (Trieste, Italy).

### Example 3 Homology Studies

The coordinates from the crystal structure are used to search the protein database for other transcription factors (such as p53, NF-κB p52, NF-κB p65, NFAT1, the T-domain and the STAT proteins) using the Dali server.

### Example 4 Generating a Model for DNA Binding by the Runt Domain

Least squares superposition of the coordinates of the STAT3β/DNA (PDB code 1bgl) co-crystal complex onto the AML1 Runt domain is used to generate a model for major groove DNA binding by the Runt domain/CBFβ heterodimer.

Results 1 Structural Studies

Overall Crystal Structure of the Runt Domain/CBFβ Heterodimer

The asymmetric unit of the crystal contains six Runt domain subunits and four CBFβ subunits arranged as two dimers of Runt domain/CBFβ heterodimers (Runt domain/CBFβ)_{2,} and, in addition, a Runt domain homodimer (FIG. 1). The molecules pack in alternate layers of heterodimers and homodimers. The final atomic model is most complete for the heterodimer CD, which includes residues 54-177 of AML1 (all the residues from the conserved Runt domain apart from the N-terminal 2 amino acids), and residues 2-135 from the CBFβ subunit. Residues 178-183 C-terminal to the Runt domain are disordered, as are residues 75-80 from CBFβ, and are not included in the model. 121 water molecules are also included in the model. The Runt domain homodimer QR (FIG. 1) has an indication of density at the interfaces where CBFβ binds in the heterodimers, but attempts to fit the CBFβ model into the density resulted in a higher R factor. This may represent partial occupancy by CBFβ subunits. The formation of (Runt domain/CBFβ)₂ dimers of heterodimers mediated by interactions between the amino-terminal residues in two Runt domains in two different crystal forms (P2₁ and P6₂ symmetry) and in 3 independent examples in the asymmetric unit of each crystal form is observed. Gel filtration studies (data not shown) are also consistent with the formation of (Runt domain/CBFβ)₂ dimers in solution. These data suggest that the formation of (Runt domain/CBFβ)2 dimers is not a consequence of crystal packing, but reflects homodimerisation of the Runt domain/CBFβ complex in solution.

### Overall Structure of the Runt Domain/CBFβ Heterodimeric Complex

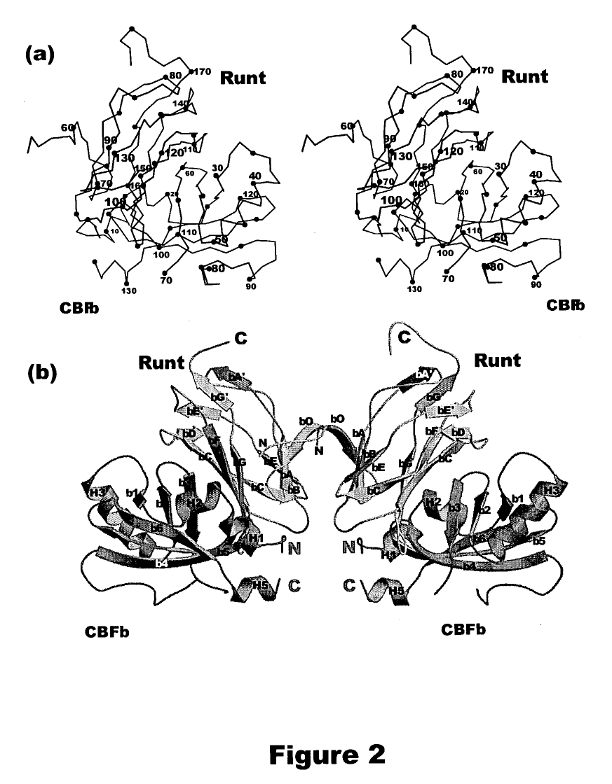

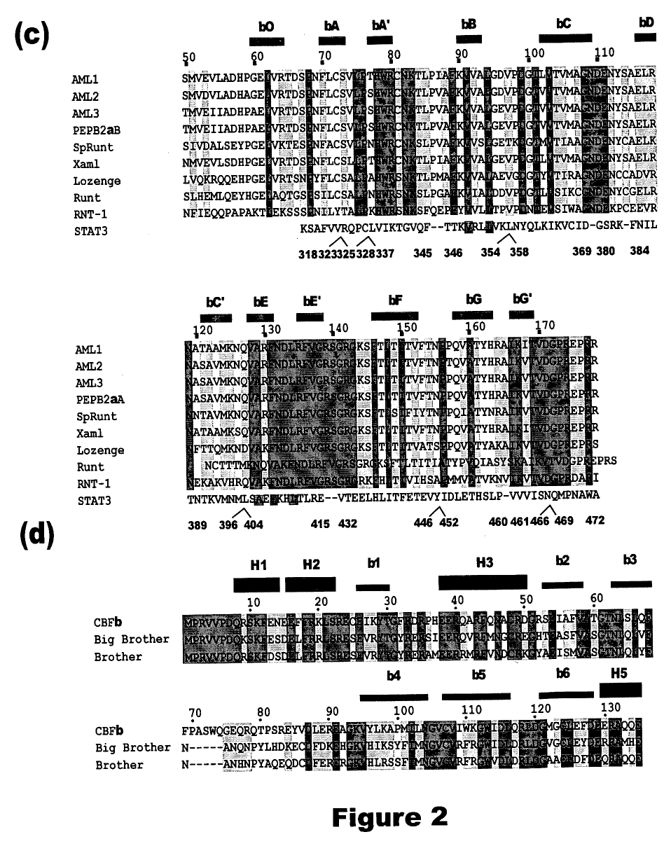

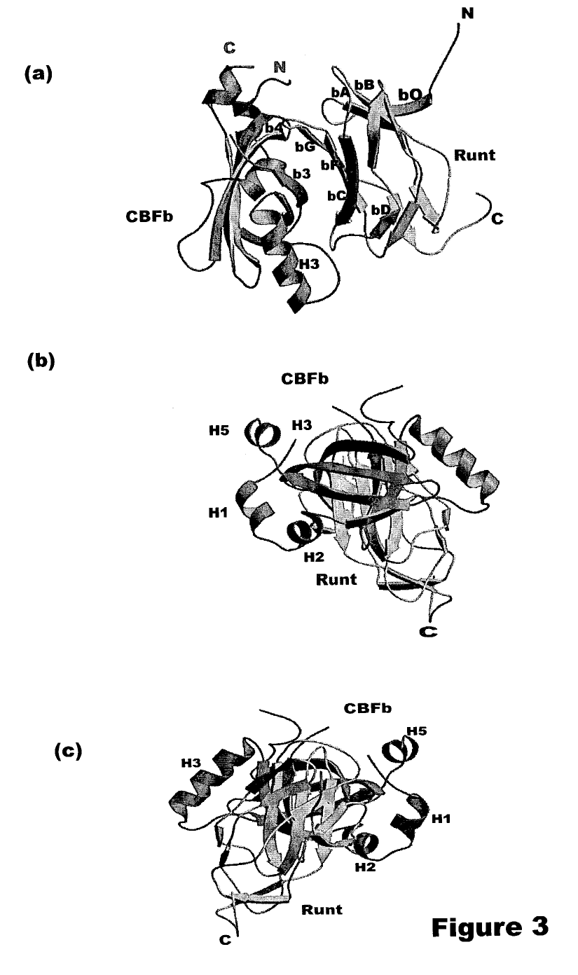

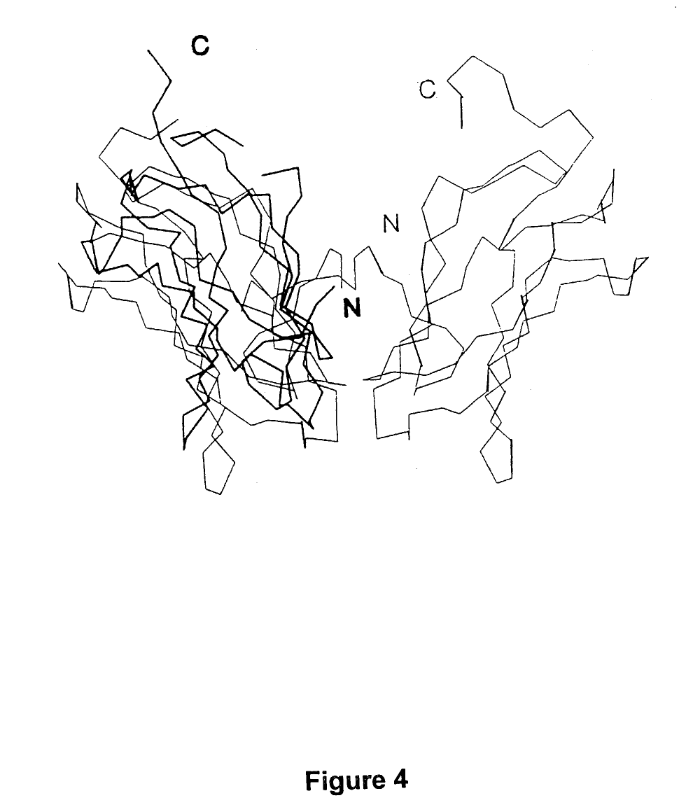

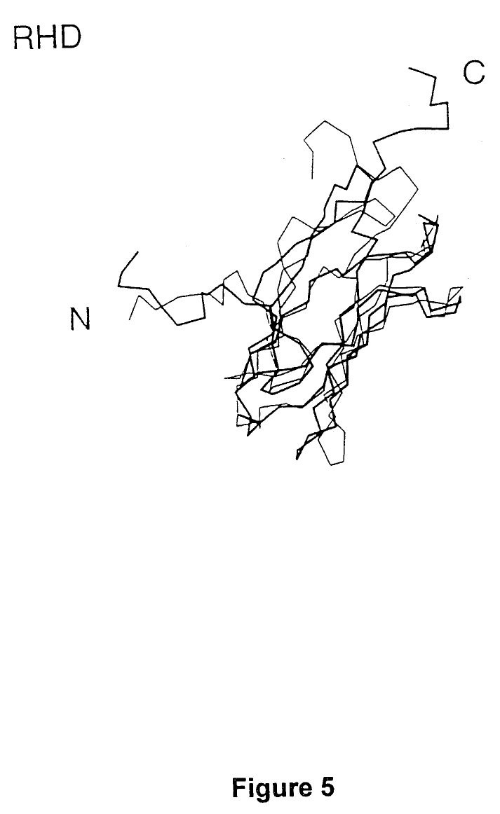

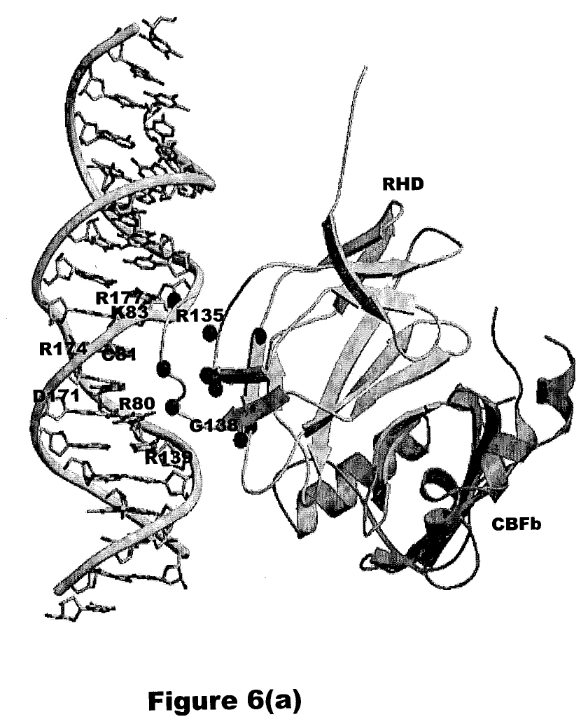

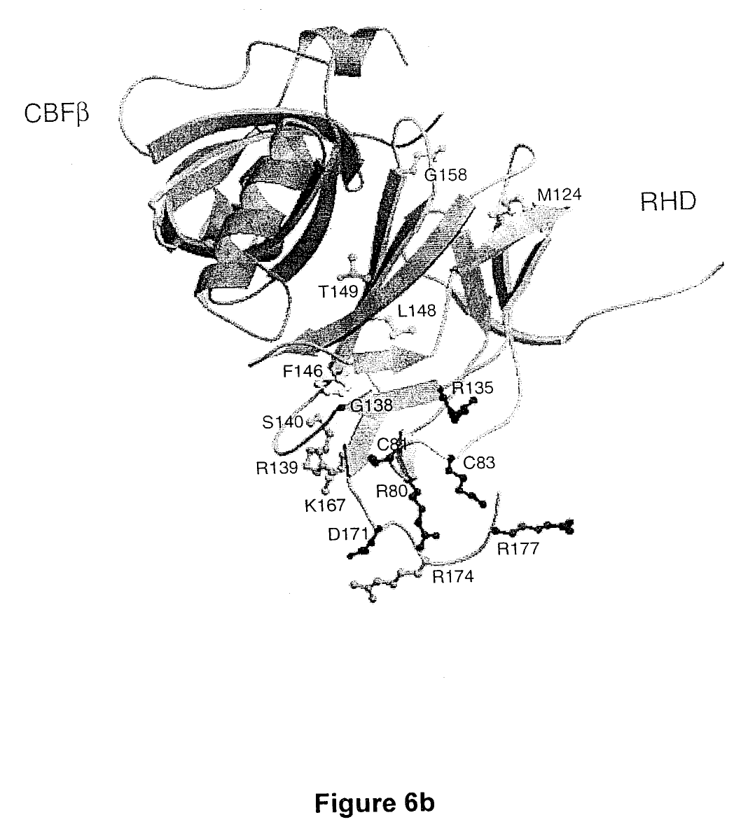

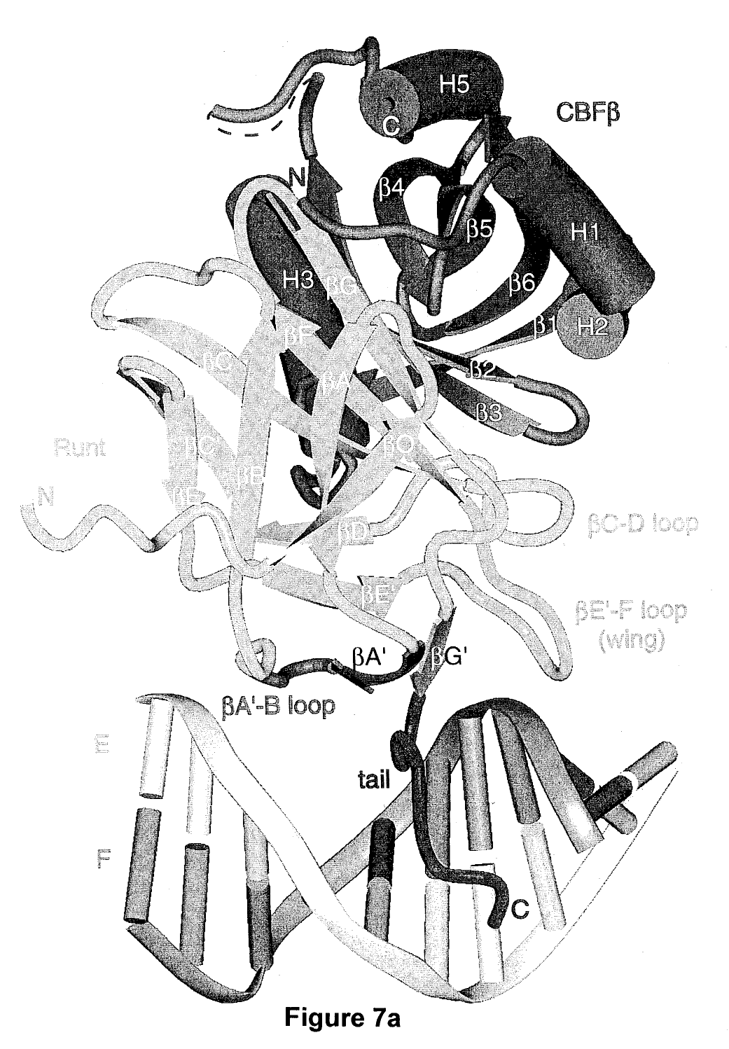

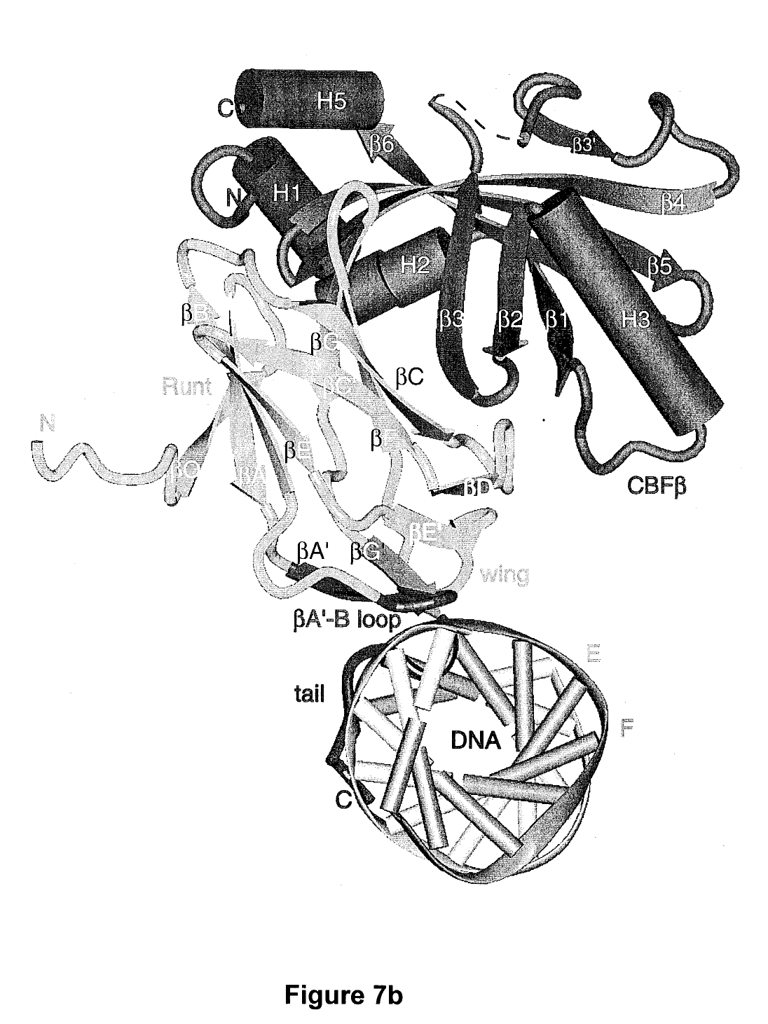

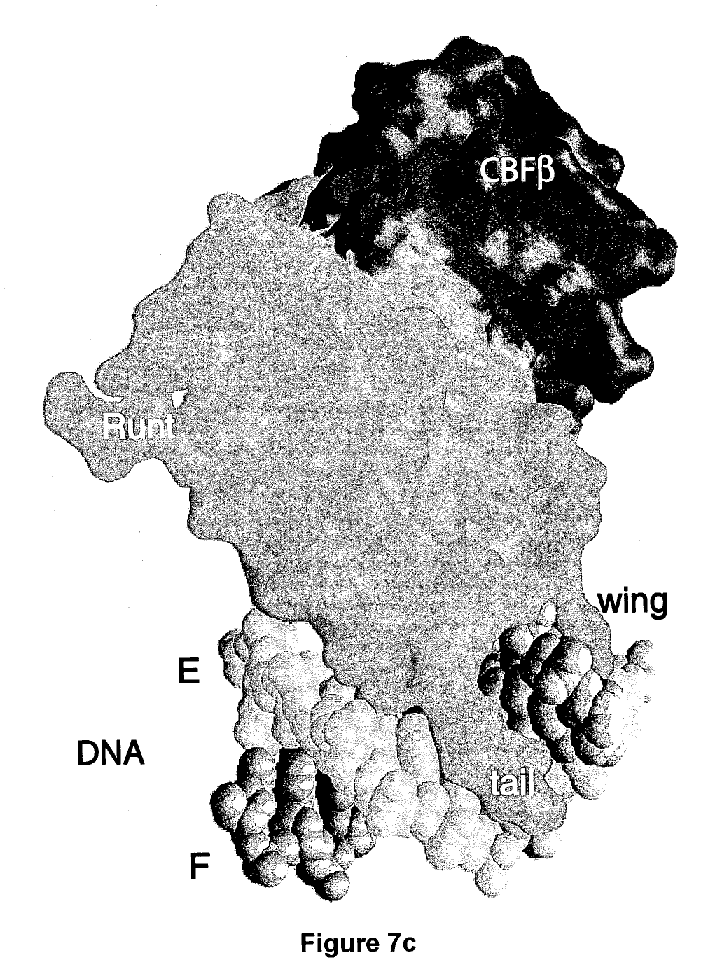

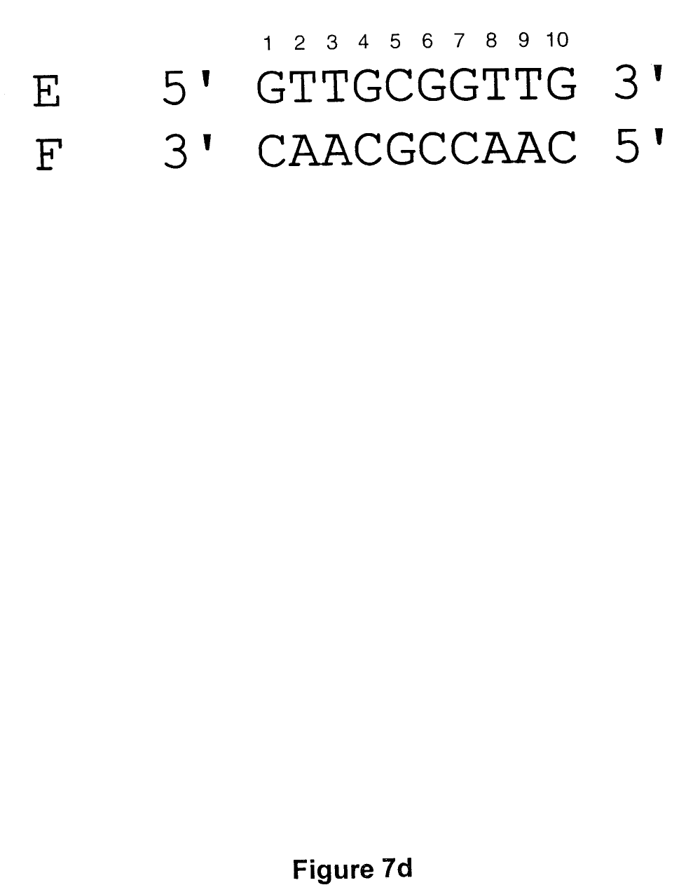

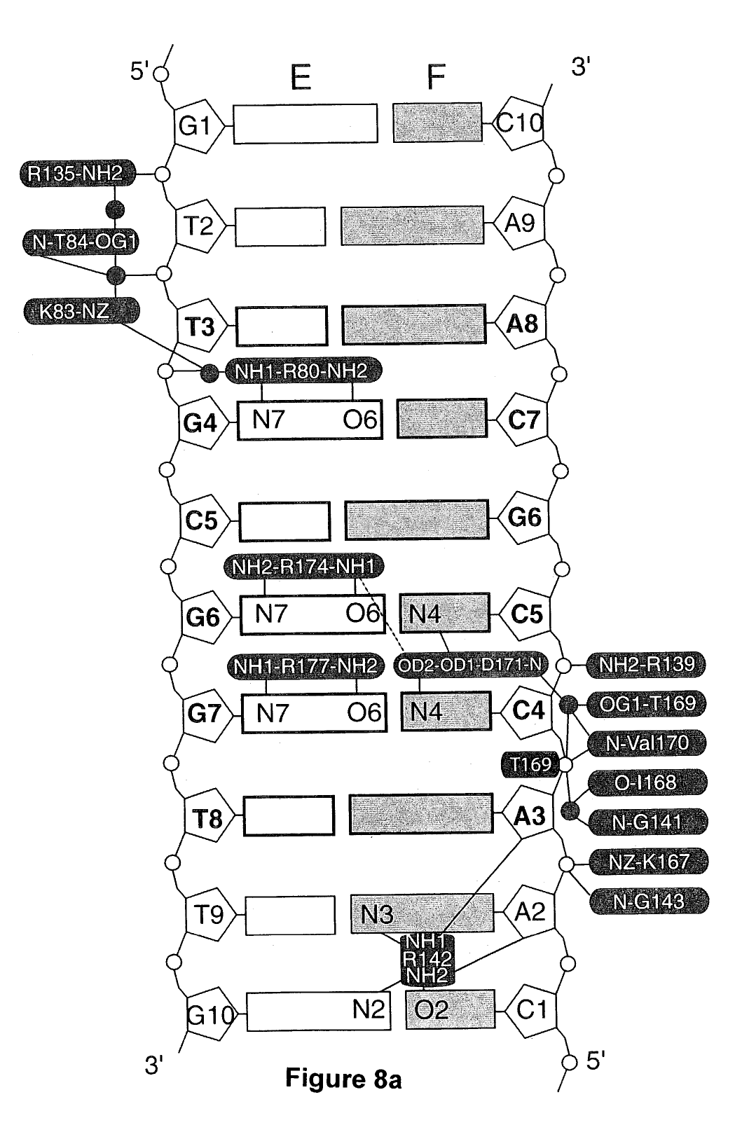

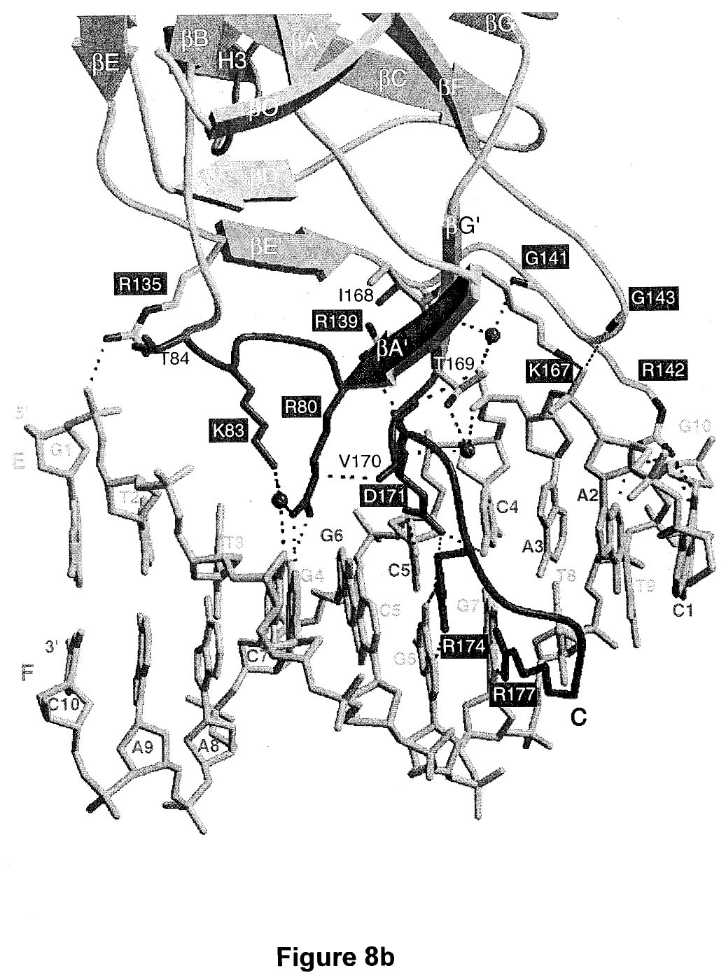

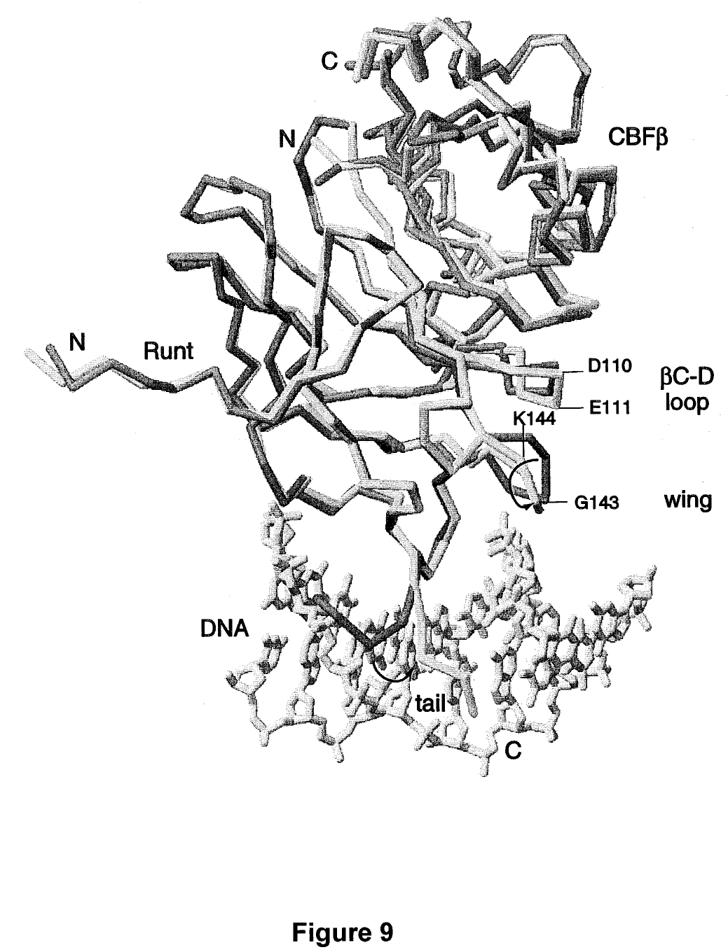

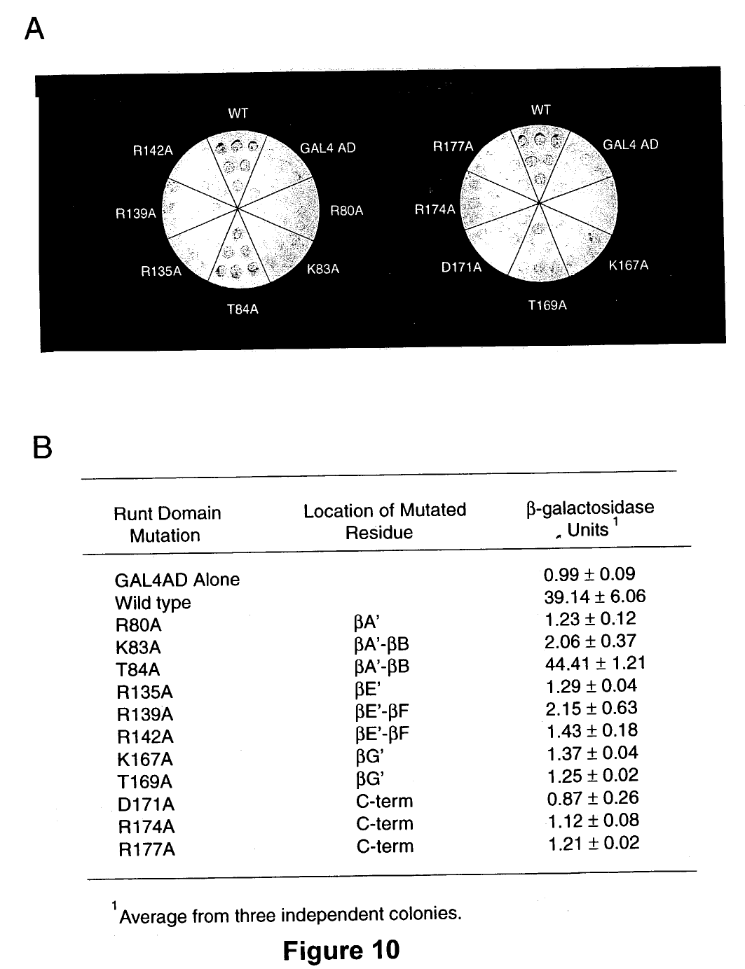

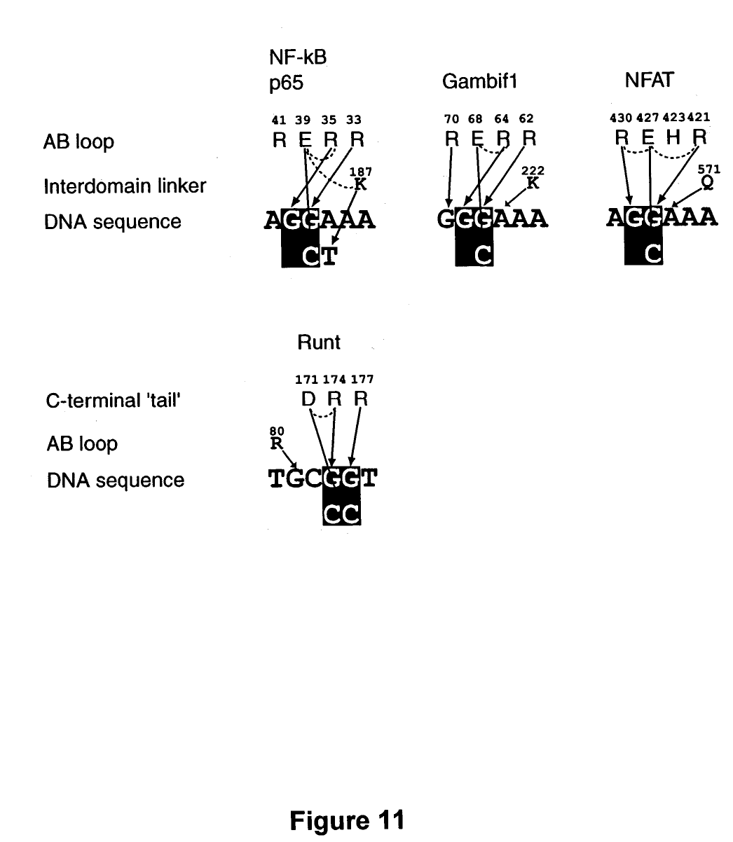

The overall structure of the (Runt domain/CBFβ)₂ dimer of heterodimers is shown in FIG. 2. Although the fold of the individual subunits of the complex are consistent with recent NMR studies (Berardi et al., 1999) (Goger et al., 1999; Nagata et al., 1999) (Huang et al., 1999), specific regions of the Runt domain differ in conformation (discussed later). The Runt domain of AML1 forms a twelve-stranded (10 antiparallel and 2 parallel strands) β-barrel which adopts an s-type immunoglobulin fold. CBFβ is a mixed α/β structure, consisting of a partly open six-stranded β-barrel with α-helices packed against the top and bottom. Although structural comparisons with the DALI database show that the β-barrel component of CBFβ has overall structural similarity to a number of functionally unrelated proteins, the fold is quite distinct, in contrast to an earlier claim (Goger et al., 1999), and is quite unrelated to the OB fold (Murzin et al., 1995). As classified in the SCOP protein structure database (Murzin et al., 1995), the OB fold consists of a 5-stranded β-barrel, with Greek key topology and a shear number of 8 or 10. By contrast, CBFβ forms a partly opened 6-stranded β-barrel with a unique combination of a meandering up and down topology of the β-strands and a shear number of 10. Consequently, this particular β-barrel structure can be classified as a novel fold from the distinct combination of topology and shear number. FIGS. 2D and E show the secondary structure elements of the Runt domain and CBFβ aligned at the top of the protein sequences.

There are extensive heterodimeric contacts at the interface between the Runt domain and CBFβ subunits, and homodimeric contacts between the amino termini of the Runt domains, but no contacts between the CBFβ subunits. The temperature factors for the (Runt domain/CBFβ)₂ structure are lowest for the Runt domain, and are relatively higher overall within the CBFβ subunit. This indicates that structurally, the Runt domain forms a relatively stable core, whereas the CBFβ subunit is more mobile.

The overall dimensions of a single Runt domain/CBFβ heterodimer are around 41×50×27 Å. There are no significant differences in the root mean square deviation (rmsd) of the Cα backbone traces between the four Runt domain/CBFβ heterodimers in the asymmetric unit of the crystal. As shown in FIG. 3, the Runt domain and CBFβ are oriented such that the long axes of the two β-barrel domains are orthogonal to one another to one another, and interact along a large continuous curved interface. When viewed from the perspective of FIG. 3B, with the C-terminus of the Runt domain oriented down, it is evident that CBFβ is related only to the upper part of the Runt β-barrel. CBFβ makes no direct contact with Runt domain loops βA-B, βE′-F or the C-terminus. Residues within these loops have been shown biochemically to be essential for DNA binding (Kagoshima et al., 1996) (Osato et al., 1999). The α-helices H5, H1 and H2 of CBFβ lie on the right lateral aspect of the upper half of the Runt β-barrel, and helix H3 lies on the upper left lateral aspect (FIG. 3B).

The data from the crystal structure differs from previous proposals for the mode of interaction between the Runt domain and CBFβ, based on chemical shift analysis (Nagata et al., 1999). The chemical shift data is interpreted by orienting CBFβ in the heterodimer with helices H1, H2 and H5 up, and helix H3 down, placing CBFβ residues Gln 74, Gln 79 and Arg 83 on the same face of the heterodimer as the proposed DNA binding surface of the Runt domain (loops βA-B and βE′-F and the C-terminus). In fact, the crystal structure demonstrates that the CBFβ is rotated by 90° relative to this proposal, placing the evolutionarily non-conserved CBFβ loop β3-β4 (residues 68-93) on the upper outer aspect of the heterodimer (FIG. 3B), with no component residues either buried in the heterodimer interface, or related to the proposed DNA binding surface of the molecule.

The conformation of the C-terminus of the Runt domain (residues 169-177), which is essential for DNA binding, is clearly defined in the crystal structure. The C-terminus shows no evidence of secondary structure, but forms a loop which extends towards the N-terminus of the Runt domain, passing below loop βA′-B (in the orientation seen in FIG. 3B). The C-terminus is not defined beyond residue 168 in the NMR structure from Nagata et al. (Nagata et al., 1999). All the loops on the lower face of the Runt domain β-barrel, as orientated in FIG. 3B (βC-D, βE′-F, the C-terminus and βA′-B), are linked to one another and are well buttressed on one side as a result of the interaction of loop βC-D with CBFβ. The C-terminus is stabilised through interactions between the flanking loops βE′-F and βA′-B. Strands βG′ and βA′ form a small parallel β-sheet, linking loop βA′-B to the C-terminus. The carbonyl of Ser 140 links loop βE′-F to Ile 168 in strand βG′ through an apparent hydrogen bond. Runt domain loop βE′-F Ser 145 interacts with loop βC-D Asp 110, thus linking the four loops on this face of the molecule. Runt domain loop βC-D, which has not been implicated in DNA binding, makes a number of contacts with CBFβ through the side chain of Tyr 113. Finally, the three Cys residues in CBFβ (Cys 25, Cys 107 and Cys 124) are not related to the heterodimer interface as proposed (Huang et al., 1999), suggesting that these residues are not directly related to the modulation of oxidation state-dependent behaviour of AML1.

Interactions at the Runt Domain-CBFβ Interface

The Runt domain and CBFβ subunits interact over a large continuous curved interface (FIG. 3), such that a total of 1900 Å² (calculated as described (Nicholls et al., 1991)) in solvent accessible surface area is buried. The interaction surface of the Runt domain is concave, and packs against a complementary convex surface on CBFβ. The curved heterodimerisation surface of the Runt domain shown in FIG. 3 involves loops βF-G, βO-A and βB-C at the top of the β-barrel; a three-stranded β-sheet (strands βC, βF and βG); and loop βC-D at the bottom of the β-barrel. The regions of CBFβ involved in heterodimerisation are the amino-terminal loop and helix II1; strand β1 and loop β1-H3; strands β2, β3 and the connecting loop β2-β3, which together form the central convex interaction surface; strand β4 and the proximal part of loop β4-β5.

The specific residues from CBFβ which make up the heterodimer interface are given in Table 2. The residues on the surface of CBFβ which are buried in the Runt domain at the interface, may be defined by the programme GRASP Nicholls et al., 1991). Of the 21 residues indicated by such a procedure, only 2 are not conserved between human CBFβ and the two related Drosophila proteins Brother and Big brother. 13 residues out of 21 are absolutely conserved, and 6 are conservative substitutions (FIG. 2E).

The Runt domain-CBFβ interaction involves the N-terminus of CBFβ (residues 2-5). The Runt domain loop βO-A, loop βB-C and strand βG are all involved in contacts with the N-terminus of CBFβ, with four potential hydrogen bonding interactions mediated by CBFβ Arg 3 (Table 2). This explains mutagenesis studies which demonstrate a requirement for the N-terminal 5-6 residues of CBFβ for heterodimerisation to the Runt domain (Golling et al., 1996) (Kagoshima et al., 1996), and is consistent with chemical shift data from Huang et al. (Huang et al., 1999). Goger et al. (Goger et al., 1999) note significant chemical shifts for Gln 74, Gln 79 and Arg 83 on binding of a Runt domain-DNA complex to CBFβ, but the crystal structure demonstrates that this is not a result of burying these residues at the heterodimer interface, and that they do not come to lie proximal to the putative DNA binding loops βE′-F or βA′-B of the Runt domain.

The contacts mediated by residues in the Runt domain at the heterodimer interface are also shown in Table 2. The residues on the surface of the Runt domain which are buried in the CBFβ subunit may again be defined by the programme GRASP (Nicholls et al., 1991). Two thirds of the residues identified by the programme are either fully conserved or conservatively substituted (FIG. 2E). Structurally, Cis-Pro 156, which is absolutely conserved, induces a kink in Runt domain loop βF-G, which makes important contacts with residues in CBFβ. The Runt domain strand βG (residues 159-161) pairs with CBFβ strand β4 (residues 102-104) to form a short antiparallel β-sheet extension. The β-sheet extension between the two subunits is stabilised by a cluster of conserved hydrophobic residues (Runt domain Val 159 and Thr 161; CBFβ Met 101, Ile 102 and Leu 103β. The sequence conservation in this region supports an important underlying function. Runt domain loop βF-G and strand βG are structurally homologous to the region of NFAT which interacts with AP1 in the crystal structure of this complex bound to DNA (Chen et al., 1998). Thus, analogous regions of other Ig fold proteins are involved in protein-protein interactions. At the opposite end of the heterodimer interface, Runt domain Ser 114 and Tyr 113 provide a large surface area of interaction with CBFβ. These residues lie in a solvent accessible polar environment, consistent with previous spectroscopic analysis (Crute et al., 1996). CBFβ residues Glu 111 and Asp 110 also contribute to this polar environment. Runt domain Met 106 makes a significant contribution to the buried surface area in the central part of the heterodimer interface. The functional importance of this residue to the interaction is supported by biochemical studies, which demonstrate that a M106V mutation abolishes the interaction between AML1 and CBFβ in vitro (Akamatsu et al., 1997b).

Out a total of 42 contacts between the two subunits, 10 represent potential hydrogen bonding interactions. Bridging contacts are also mediated indirectly via water molecules near the interface. We have identified 8 water molecules buried at the heterodimer interface, the role of which is presumably to optimise the complementarity of the interaction interfaces. The surface residues of the Runt domain identified as buried in CBFβ at the heterodimer interface is generally consistent with the chemical shift data of Nagata et al. (Nagata et al., 1999).

Homodimer Interface of the Runt Domains

The crystal structure identifies two homodimeric interactions between the Runt domains, one interface between the Runt domains in the dimer of heterodimers, and a smaller interface involving a subset of these interactions within the homodimer QR (890 Å² solvent accessible surface area buried as opposed to 1300 Å²; determined using GRASP (Nicholls et al., 1991)). The interfaces are shown in FIG. 2. The N-terminal residues (59-66) make the most prominent contribution to this interface, stabilised further by contacts between strands βA and βB, and from loops βB-C and βC′-E.

The residues buried in the homodimer interface may be identified using the GRASP programme. Runt domain Asp 66 contributes to both the heterodimer and homodimer interfaces. The homodimeric interaction in the dimer of heterodimers is mediated by a short edge to edge anti-parallel β-sheet formed from the pairing of residues 60-62 at the amino-terminal end of the Runt domain, which is stabilised by a hydrophobic cluster involving Pro 59, Leu 62, Val 63, Leu 71, Val 92, and Leu 94 from each subunit. The hydrophobic core of the interface shows strong sequence conservation (FIG. 2D). Four potential hydrogen bonding interactions also contribute to this interface, in addition to those mediating the β-sheet interaction. Superposition of the Q:R homodimer onto C:A (FIG. 4) demonstrates that the R subunit in the Q:R homodimer is relatively rotated and tilted away from the contact interface with respect to the A:C dimer. This suggests that there is flexibility between the RHD surfaces at the homodimer interface.

Electrostatic Surface Potential of the Runt Domain/CBFβ Heterodimer

There are two contrasting surfaces on the heterodimer in terms of the electrostatic surface potential. A strongly basic surface corresponds to the position of loops βA-B, βE′-F and the C-terminus. This supports the biochemical and human mutation data which directly implicate these regions of the Runt domain in DNA binding. In contrast, rotation by 145° from this region reveals a strikingly acidic surface, corresponding to the upper outer surface of CBFβ. Five areas on the surface of CBFβ make up this charged surface: the end of strand β6 and the residues prior to helix H5; residues in loop β3-β4; the β5-β6 loop; strand β1; and helix H1. The majority of these residues are conserved in evolution (FIG. 2E), suggesting that they are functionally important.

Conformational Differences between the Runt Domain Bound to DNA and Bound to CBFβ

Interaction with CBFβ is essential for the in vivo function of AML1 (Sasaki et al., 1996) (Wang et al., 1996b) (Wang et al., 1996a). Biochemically, CBFβ decreases the dissociation constant (K_{d}) of the isolated Runt domain for its cognate DNA-binding site 5-10 fold (Kagoshima et al., 1996). The molecular basis for this enhanced DNA binding affinity of the Runt domain in the presence of CBFβ is not clear. The availability of structures of the Runt domain/CBFβ binary complex (this work) and the coordinates for the NMR structures of the Runt domain (performed in the presence of DNA) (Nagata et al., 1999) and the bundle of NMR structures of the uncomplexed CBFβ subunit (Huang et al., 1999) (Goger et al., 1999) this issue to be addressed.

No significant differences in the crystal structure of CBFβ were found with respect to available NMR coordinates (Huang et al., 1999) (Goger et al., 1999) (PDB codes 2jhb and 1c13), apart from the disorder in the flexible non-conserved loop β3-β4 (residues 75-80) in the heterodimer. The coordinate precision is high (0.59 Å²) for the backbone residues of the bundle of Runt domain NMR structures (residues 62-170) for which coordinates are available (PDB code lcmo). The Cα traces for the structures of the CBFβ- and DNA-bound forms of the Runt domain superimpose well for the most part (FIG. 5A). In the region of the putative DNA binding loops, however, the structures differ significantly. In the Runt domain-CBFβ complex, the C-terminal residues of the Runt domain are ordered, defining the conformation of this essential DNA binding region. The small parallel β-sheet (βA′-βG′) linking Runt domain loop βA′-B and the C-terminus is shifted significantly towards the βE′-F loop. The conformation in the crystal structure is stabilised by the potential main chain hydrogen bonding interactions between loop βE′-F (Arg 139 and Gly 141) and the Runt domain C-terminus (Ile 168). Cys 81 forms potential hydrogen bonding interactions linking loop βA′-B (Cys 81) to strand βG′ (Thr 169) and to strand βE′ (Val 137), following a shift of 7.2 Å in its position (the largest change in relative position of any Cα). This change in chemical environment would be expected to alter the susceptibility of Cys 81 to oxidation. It has been proposed that the oxidation state of Runt domain Cys 81 may be modulated as a result of CBFβ-induced conformational changes (Akarnatsu et al., 1997a), and the structural data from the crystal supports this hypothesis. In the NMR structure, Runt domain loops βC-D and βE′-F are more closely related, with potential hydrogen bonding interactions between Asp 110 (loop βC-D) and Ser 145 (loop βE′-F) not evident in the CBFβ-Runt domain complex. To accommodate the movement of the βA′-βG′ sheet, there is a bulge in the main chain conformation from residues 85-89 (Pro 86 is shifted by 5.9 Å), and an upward kink at Ala 165 (shifted by 6.5 Å), relative to the NMR structure. The kink at Ala 165 is stabilised by main chain potential hydrogen bonding with Tyr 162. Tyr 162 projects into the hydrophobic core of the molecule, but the residues immediately adjacent (Thr 161 and His 163) are buried in the CBFβ subunit at the heterodimer interface, suggesting the possibility of induced conformational change transmitted from the heterodimer interface by stabilisation of the orientation of the Tyr 162 side chain. In addition, there is a change in the conformation of the Runt domain βF-G loop (residues 153-159), which is twisted through a 90° angle in the crystal structure, resulting in displacement of Pro 156 by 6.15 Å relative to its position in the NMR structures. As discussed above, this region of the Runt domain forms extensive contacts with CBFβ. These comparisons demonstrate ordering of the C-terminus of the Runt domain in the presence of CBFβ and conformational flexibility within the Runt domain which can alter the relative orientation of a specific DNA-binding subdomain. This suggests that the promotion of a specific conformation of the AML1 Runt domain DNA-binding surface on binding to CBFβ may provide one mechanism for enhanced DNA binding by the Runt domain-CBFβ complex.

Results 2 Mapping of Known Mutations to the Structure

Mutations in Cleidocranial Dysplasia Map to Heterodimer Interface

Congenital point mutations within the Runt domain of AML1 and AML3 have been attributed to the pathogenesis of congenital familial platelet disorder which progresses to acute myeloid leukaemia (AML1) (Song et al., 1999), and to a disorder of osteogenesis, cleidocranial dysplasia (AML3) (Lee et al., 1997) (Mundlos et al., 1997). Somatic mutations in AML1 have also been demonstrated in acute leukaemia (Osato et al., 1999). The majority of point mutations involve residues directly implicated in DNA binding, but recently described missense mutations associated with skeletal dysgenesis map to the heterodimer interface (Zhou et al., 1999) (Quack et al., 1999) (FIG. 7A).

Three mutations affect residues identified in the crystal structure as buried at the heterodimer interface: T149A, Q158R and S67R (FIG. 6A and Table 2), and map to the Runt domain strands βF, strand βG and loop βO-A respectively. The T149A mutation is associated with variable expressivity in a single family, ranging from classical CCD, to dental anomalies alone, while the Q158R and S67R mutations are associated with classical CCD (Zhou et al., 1999). The T149A mutation would be expected to disrupt a potential salt bridge with CBFβ Asn 63. Q158 interacts with CBFβ, but also stabilises the conformation of Runt domain loops βO-A and βF-G through potential salt bridges. The Q158L mutation in vitro results in impairment of DNA binding and loss of heterodimerisation (Lenny et al., 1995). Q158R would also be expected to disrupt potential salt bridges. These mutations demonstrate that the conformation of Runt domain loops βF-G and βO-A is crucial both for interaction with CBFβ and for DNA binding, despite being located on the opposite face of the β-barrel to the putative DNA binding surface.

The crystal structure defines the conformation of the Runt domain C-terminus, which is essential for nuclear localisation and DNA binding (Quack et al., 1999) (Kagoshima et al., 1996) (Osato et al., 1999). Mapping mutations associated with FDP, AML and CCD onto the structure clearly delineates the potential DNA binding surface (FIG. 7A). One of the mutations described in FPD, R139Q, which is also described in CCD, has indeed been shown to confer loss of function in vitro (Zhou et al., 1999). Arg 174 is the residue most frequently affected by mutation in the AML3 Runt domain in a recent CCD study (Quack et al., 1999), which also demonstrated that the mutation R174Q abolishes DNA binding in vitro, and that R174Q and R174W disrupt the function of the nuclear localisation signal in accumulating AML3 in the nucleus.

The structure of the heterodimer interface is consistent with the in vitro Runt domain mutants M106V, G108R and N109D, which show loss of heterodimerisation but preservation of DNA binding activity. Mutations in the Runt domain residues 66-69 located on the βO-A loop, also abolish heterodimerisation. Recent in vivo experiments in Drosophila demonstrated that the G108R Runt protein mutant is also dysfunctional in several in vivo assays, showing that the interaction of Runt with the CBFβ homologues Brother and Big brother is essential in vivo for the function of the core binding factor complex.

Results 3 Homology Modelling with Other Transcription Factors

The structural homology of the AML1Runt domain to transcription factors p53, NF-κB p52, NF-κB p65, NFAT1, the T-domain and the STAT proteins has been noted (Nagata et al., 1999) (Berardi et al., 1999). The coordinates from the crystal structure are used to search the protein database using the Dali server, and this showed that Runt domain is most closely related to the transcription factor STAT3β. Structural alignment of the Runt domain with STAT3β gives an rmsd of 2.1 Å over 82 residues (see FIG. 2C). The structural homology is prominent even within the loop regions of the Runt domain, most strikingly within the C-terminus. The smaller size of the Runt domain DNA-binding module is related to the shorter length of the putative DNA binding loops. Loops analogous to Runt domain loop βA-B which are found in the five other related transcription factors, mediate important DNA contacts in the major groove. NMR studies have demonstrated NOEs from Runt domain Arg 80 in the βA-B loop to the DNA (Nagata et al., 1999), and the mutation R80C abolishes DNA binding (Osato et al., 1999). The residues in STAT3β structurally equivalent to Runt domain Arg 80, Lys 83 and Thr 84 mediate DNA binding contacts. Runt domain Arg 80 has structural equivalents in the analogous βA-B loops of NF-βB and NFAT1 which make direct contacts with the DNA. Although the Runt domain is closely related to STAT3β structurally, there is no evidence that the mode of DNA binding is other than monomeric (Ogawa et al., 1993a).

Results 4 Modelled Structure of the AML1 DNA Binding Site

The crystal structure may be used to generate a model for major groove DNA binding by the Runt domain/CBFβ heterodimer, by least squares superposition of the coordinates of the STAT3β/DNA (PDB code 1bgl) co-crystal complex onto the AML1 Runt domain (FIG. 6B). In this model, the orientation of the Runt domain/CBFβ complex with respect to the DNA is consistent with other data: the location of human disease mutations (FIG. 6A); the electrostatic surface potential of the heterodimer; in vitro mutagenesis data (Kagoshima et al., 1996) (Lenny et al., 1995) (Osato et al., 1999); chemical footprinting analysis (Thomell et al., 1988) (Melnikova et al., 1993); limited NOE data from NMR studies (Nagata et al., 1999); and the observations that CBFβ does not contact DNA directly, or extend the Runt domain footprint on DNA (Kamachi et al., 1990). Three loops from the Runt domain β-barrel are predicted to make close contacts with the DNA, from the model. The βA-B loop and the C-terminus are predicted to participate in core sequence recognition in the major groove. The conformation of the C-terminus, which interacts with loop βA-B through the small parallel β-sheet, is a conserved structural feature between the related Ig fold transcription factors. The model of the present invention predicts that loop βE′-F would make DNA backbone and minor groove contacts. The analogous segment in STAT3β is involved in DNA backbone interactions (Becker et al., 1998).

The S140N mutation abolishes DNA binding by the Runt domain (Lee et al., 1997). Ser 140 is a conserved residue which stabilises the conformation of the βE′-F loop through potential hydrogen bonding interactions within the βE′-F loop and mediates main chain interactions from loop βE′-F to the C-terminus. The S140N mutation is predicted to alter the potential hydrogen bonding interactions, and is predicted to have similar consequences to the in vitro mutant S140G, which abolishes both the DNA binding and heterodimerisation functions of the Runt domain (Akamatsu et al., 1997b). The mutation G138D (P. Femaux, personal communication), associated with AML, affects a residue just proximal to the critical βE′-F loop, introducing a charged side chain into the hydrophobic core of the molecule. Analogous mutations, which are predicted to alter the flexibility of the βE′-F loop, disrupt the DNA binding function of STAT3β and STAT6 (Becker et al., 1998). These data suggest that the degree of flexibility of the loops on the lower face of the Runt domain β-barrel is a critical determinant both of DNA-binding and heterodimerisation functions of the Runt domain. This suggests that an important mechanism through which CBFβ alters the DNA binding affinity of the Runt domain is by buttressing the DNA-binding loops and stabilising their conformation.

Berardi et al. have noted NOEs to DNA from Runt domain loop βF-G in their NMR studies (Berardi et al., 1999). However, in the crystal structure of the heterodimeric complex, residues on the surface of Runt domain loop βF-G (cis-Pro 156, Pro 157, Gln 158) are clearly buried within the CBFβ subunit (FIG. 4B, and Table 2). Runt domain loop βF-G and strand βG are structurally homologous to the region of NFAT which interacts with AP1 in the crystal structure of this complex bound to DNA (Chen et al., 1998), and the model of the present invention is compatible with existing footprinting data which demonstrates that the AML1/CBFβ complex protects about 10 base pairs of DNA (Thornell et al., 1988) . The model therefore suggests that the core binding factor heterodimer binds DNA through Runt domain loops βE′-F, βA′-B, and the C-terminus.

Example 5 Crystal Structure of Ternary Complex

A single AML1 Runt domain-CBFβ heterodimer binds to the major and minor grooves of the 10 base pair (bp) target oligonucleotide, making potential hydrogen bonding interactions to the bases and nucleotide backbone of the DNA (FIG. 7). Overall, the dimensions of the complex are around 50×62×24 Å. One face of the Runt domain immunoglobulin β-barrel binds to the DNA, but CBFβ makes no DNA contacts. The overall fold of the Runt domain-CBFβ heterodimer is consistent with our previous report (Warren et al., 2000), but two structural adaptations ensure that the Runt domain has an extended interaction with the DNA. The C-terminal ‘tail’ and the ‘wing’ of the Runt domain change conformation dramatically to clamp the DNA, which is itself significantly bent towards the protein within the consensus AML1 binding site.

AML1 Runt Domain

The Runt domain twelve-stranded β-barrel is orientated with the long axis perpendicular to the DNA helical axis (FIG. 7). All the structural elements that contact the DNA project from one face of the β-barrel. The Runt domain uses two loops, a β-sheet, a β-strand and the extended C-terminal ‘tail’ to contact DNA. The βA′-B loop (residues 81-89), the βA′-G′ sheet (βA′-residues 77-80; βG′-residues 166-169), the βE′ strand (residues 135-138) and the C-terminal ‘tail’ (residues 170-177) dock in the major groove and make contacts to the edges of the DNA base pairs and nucleotide backbone. The βE′-F loop (‘wing’) (residues 139-145) provides an arginine that fits into the minor groove. Residues from the β-A′-G′ sheet, the ‘tail’ and the ‘wing’ contact the nucleotide backbone that divides the major and minor grooves, forming a continuous surface which clamps around the DNA like the claw of a crab (FIG. 7C). The C-terminal ‘tail’ and the ‘wing’ significantly enhance the overall surface area of contact with the DNA in the major groove, burying a total of 1519 Å² in solvent-accessible surface area (assuming default radii of GRASP and a 1.4 Å solvent probe) (Nicholls et al., 1991).

CBFβ

The structural model for CBFβ in the ternary Runt domain-CBFβ-DNA complex contains an additional short β-strand (residues Y85-D87), designated β3′ (FIG. 7B). Strand β3′ extends the antiparallel β-sheet formed by β-strands β4, β5 and β6 through hydrogen bonding interactions with strand β4 (residues K98-T96). Strand β340 was also present following further refinement of the Runt domain-CBFβ heterodimer data, but is found in a part of the structure associated with high temperature (B) factor values. A previous NMR study documents the presence of a 3₁₀-helix (H4) involving CBFβ residues R83-R90 (Werner et al., 1999). The differing secondary structures seen in these studies, and the associated high B factors, suggest that this region is conformationally flexible. Our current model for CBFβ comprises a seven-stranded β-barrel with four peripheral α-helices. CBFβ makes neither direct nor indirect contacts with the DNA (FIG. 7). However, CBFβ loop β2-3 projects down from the central part of the molecule to stack above Runt domain loop βC-D, which in turn packs against Runt domain loop βE′-F (FIG. 7B). Mutational analysis has demonstrated a critical energetic and functional role for CBFβ loop β2-3 in heterodimerisation with the Runt domain (Tang et al., 2000b).

DNA Conformation in the Complex

The two DNA duplexes (designated EF and GH) in the asymmetric unit of the crystal have nearly identical structures, with an all atom root mean square deviation (rmsd) of 0.171 (for 404 atoms). Table 2 lists various parameters describing the structure of DNA oligonucleotide EF. The DNA duplex adopts an overall B-DNA-like conformation, with slight underwinding (10.8 base pairs per turn), and an average helical rise of 3.27 Å, however, there are deviations from standard B-DNA geometry. The most striking is the marked inclination between base pairs G4-C5 (18.8 degrees), G7-T8 (18.5 degrees), and T9-G10 (17.3 degrees) on strand E. These base-pair steps have associated high roll values, resulting in significant bending of the DNA towards the protein (by about 40°). The major groove is wide (18.3 Å), and the minor groove is enlarged (13.4 Å) in the region of sequence-specific base contacts by the Runt domain. This type of DNA structure, which has been noted in other unrelated protein-DNA complexes, has been classified as a B_{eg}-DNA (where eg means enlarged groove) subclass DNA (Nekludova and Pabo, 1994). Thus, the DNA conformation in the Runt-CBFβ-DNA complex shows similarities to the otherwise unrelated glucocorticoid, MetJ, engrailed, and Tramtrack protein-DNA complexes (Nekludova and Pabo, 1994), suggesting that the DNA itself plays an important role in the protein-DNA recognition.

Mechanism of DNA Recognition

Backbone Contacts

The Runt domain is anchored on either side of the major groove through direct and water-mediated contacts with the regions of the phosphate backbone that flank and partly overlap the core recognition sequence (FIG. 8). The network of interactions is essentially the same for both ternary complexes in the asymmetric unit, with the exception of T84, which makes water-mediated contacts to the phosphate backbone of DNA duplex GH only. The potential neutralising contacts with the phosphate groups lie on one face of the DNA helix, and involve consecutive phosphates on either side of the major groove. This may encourage DNA bending towards the protein surface as a result of phosphate neutralisation. At the 5′ end of the consensus-binding site, residues from strand βE′ (R135) and loop βA′-B (R83, T84) contact backbone nucleotides of DNA strand E (T2, T3 and G4) (FIG. 8A). Backbone nucleotides on the opposite DNA strand F (A2, A3, C4, C5), are contacted by residues from the ‘wing’ (R139, G141, R142, G143), strand βG′ (I168, T169) and the C-terminus (V170) (FIG. 8A). R142 from the ‘wing’ makes additional contacts to bases from both strands in the minor groove (strand E, base G10; strand F, base A2). The amino acid sequence between strands βE and βF contains the longest stretch of conserved sequence identity in the Runt domain, supporting a critical role for these residues in the DNA binding mechanism.

Base Pair Contacts

The two Runt domain-CBFβ heterodimers in the asymmetric unit show the same pattern of base-specific contacts (FIGS. 2A and B). The primary DNA binding specificity determinants localise to strand β-A′ (R80), and the C-terminal ‘tail’ (D171, R174 and R177). In particular, the side chain atoms NH1 and NH2 from R80 (strand βA′) form potential bidentate hydrogen bonding interactions with nitrogen N7 and the exocyclic oxygen 06, respectively, of base G4 (strand E). Two other interactions anchor the position of the R80 side chain and guarantee specificity. R80 (atom NE1) forms a potential hydrogen bond with the main chain carbonyl oxygen of V170 (FIG. 8B), and a potential water-mediated hydrogen bond (through atom NH1) to base G4 (backbone atom O1P) on strand E. The side chain of K83 makes a similar potential indirect backbone contact to base G4, further stabilising the position of the R80 side chain.

The Runt domain C-terminal ‘tail’ makes the most extensive set of base contacts. R174 (atoms NH1 and NH2) makes a potential bidentate hydrogen bond to atoms O6 and N7, respectively, of base G6 (strand E); R177 (side chain atoms NH1 and NH2) makes potential bidentate hydrogen bonds to atoms N7 and O6, respectively, of base G7 (strand E). D171 (side chain atoms OD1 and OD2) forms potential hydrogen bonding interactions with the two N4 nitrogen atoms on the complementary bases C4 and C5 (strand F). D171 also makes a potential water mediated contact to the O1P backbone atom of base C4 (strand F), and a stabilising potential salt bridge to R174 (side chain atom NH1). Thus, the orientation of the R174 and D171 side chains are precisely fixed by a buttressing network of hydrogen bonds, which further enhances the specificity of the DNA interaction.

DNA-induced Conformational Changes in the Runt Domain-CBFβ Complex

Overall, the Cα traces for the Runt domain-CBFβ-DNA ternary complex and the Runt domain-CBFβ heterodimer superimpose well, with an rmsd of 0.71 over 242 atoms (FIG. 9). However, this comparison reveals marked DNA-induced conformational changes in the βE′-F loop (‘wing’) and the C-terminal ‘tail’ of the Runt domain.

The most dramatic conformational change in the presence of DNA occurs within the extended C-terminal ‘tail’ that makes the majority of the sequence-specific DNA contacts. Beyond P173, the ‘tail’ undergoes a 180-degree rotation to align R174 and R177 with bases G6 and G7 (strand E), respectively. The extended ‘tail’ runs down through the major groove, showing striking surface complementarity to the DNA.

Within the ‘wing’, the most striking shift in the position of any Cα (7 Å) is the main chain nitrogen of residue G143, which forms a potential hydrogen bond to the nucleotide backbone of DNA strand F (base A3, atom O2P) in the ternary complex. The position of the R142 side chain also changes significantly, enabling it to make backbone and base contacts in the minor groove. The entropic cost of locking the Runt domain βE′-F loop into an alternate conformation may be offset by the formation of additional polar interactions by residues within the loop. This hypothesis is supported by the fact that the side chain of K144 (loop βE′-F), disordered in the absence of DNA, is clearly defined in the ternary complex. K144 makes potential salt bridges to the side chains of D110 and E111 (loop βC-D), thus bridging the ‘wing’ and the βC-D loop of the Runt domain (FIG. 9). Mutation analysis supports an important structural role for K144 (Lenny et al., 1995; Crute et al., 1996). A further novel potential hydrogen bond is formed between N109 (βC-D loop) and the carbonyl oxygen of S140 (βE′-F loop). Runt domain S140 (βE′-F loop) forms a novel potential hydrogen bond to the carbonyl oxygen of K144. Thus, S140 bridges Runt domain loops βC-D, βE′-F, and the parallel βA′-G′ sheet which makes base-specific contact to the DNA. The mutants S140G and N109D show loss of DNA binding and heterodimerisation, demonstrating the functional importance of these residues (Akamatsu et al., 1997).

The Kd for the CBFβ-Runt domain interaction in solution (5.2±1.2×10⁻⁷ M) is 10 fold higher than the Kd for the CBFβ interaction with the DNA-bound Runt domain (4.5±0.5×10⁻⁸ M) (Tang et al., 2000a). This suggests that one or both proteins undergo a conformational change when the heterodimer binds DNA. Comparison of the Runt domain-CBFβ-DNA complex with the Runt domain-CBFβ complex identifies local conformational changes between CBFβ loop β2-3 and Runt domain loop βC-D (FIG. 9). In particular, the bulky aromatic side chain of Y113 changes conformation, accommodated by reorientation of the side chain of CBFβ N63. As CBFβ loop β2-3 makes an important energetic and functional contribution to the heterodimer interface (Tang et al., 2000b), conformational change at this interface offers a possible explanation for the change in the free energy of interaction between CBFβ and the Runt domain in the presence and absence of DNA.

Mutational Analysis of DNA Contact Residues

Alanine residues were substituted for all the side chains implicated in contacting the DNA from the structural analysis. DNA binding was monitored using a yeast-one-hybrid assay. Substitution with alanine should remove interactions with DNA mediated by atoms beyond the β carbon of an amino acid side chain.

The mutant Runt domain proteins were assayed for their ability to interact with a high affinity consensus DNA binding site in a yeast one-hybrid assay. Mutant Runt domain proteins were expressed as fusions to the GAL4 activation domain. The plasmids expressing the fusion proteins were transformed into a yeast strain containing a lacZ reporter gene under the regulation of three tandem repeats of the high affinity binding site, and both filter and liquid β-galactosidase (β-gal) assays were performed (FIG. 10). Alanine substitution of all side chains directly contacting DNA bases or the nucleotide backbone in the structure (including R80, R135, R139, R142, K167, D171, R174, and R177), severely perturb DNA binding, reducing it to background levels in the yeast assays. Two side chains participating in water-mediated contacts, K83 and T169, were also sensitive to alanine substitution. Substitution of T84 with alanine did not affect DNA binding. However, the water-mediated contact by T84 to DNA was only observed in one of the two ternary complexes in the crystal.

The structural and mutational analysis provides a firm basis for understanding the determinants of DNA binding specificity in the AML1 Runt domain-CBFβ-DNA ternary complex and the molecular consequences of human disease mutations for the DNA binding function. The structure shows that the architecture of the DNA bound Runt domain-CBFβ complex is quite distinct from other members of the immunoglobulin superfamily of transcription factors, yet there is remarkable conservation of the base-specific contacts with the Rel group of transcription factors. Structural and functional similarities to p53 have implications for understanding the DNA binding mechanism of the Runt domain-CBFβ complex.

Residues Mutated in Human Disease Make Critical DNA Contacts

Acquired somatic mutations in the AML1 gene are described in patients with leukaemia and myelodysplasia, with a particularly high incidence of mutations in MoAML and myeloid malignancies with acquired trisomy 21 (Osato et al., 1999; Imai et al., 2000; Preudhomme et al., 2000). Congenital mutations in AML1 are described in individuals with FPD/AML, and in the related AML3 gene in the congenital disorder of osteogenesis, cleidocranial dysplasia (CCD) (Lee et al., 1997; Quack et al., 1999; Song et al., 1999; Zhou et al., 1999).

A key finding that emerges from the structure of the ternary complex is that most of the disease-associated missense mutations which map to the Runt domain affect residues involved in sequence-specific base interactions or nucleotide backbone contacts (FIG. 8B). Leukaemia-associated mutations target most of the residues which contact DNA in the structure: R80C, K83N, R135G, R139Q, D171G, R174Q, R174G, R177G, R177Q and R177ter (R177 mutated to a termination codon). All of these mutations would potentially disrupt critical hydrogen bonding interactions to the DNA nucleotide backbone (K83N, R135G, R139Q) or bases (R80C, D171G, R174Q, R174G, R177G, R177Q). Interestingly, R177ter and R177Q showed barely detectable DNA binding by themselves, but in the presence of CBFβ, they produced supershifted bands with markedly increased intensities (Osato et al., 1999). Restoration of significant DNA binding activity in these mutants in the presence of CBFβ may be explained by stabilisation of the DNA-induced conformational changes in loops βC-D and βE′-F.

R80C was identified in a patient with blast transformation of chronic myeloid leukaemia, and K83N in a patient with relapsed AML M3 (Osato et al., 1999). The R174Q mutation was reported in the context of FPD/AML, and R174 was the residue in AML3 (Runx2/CBFA1) that was most frequently affected by congenital mutations (including R174Q, R174W) in a study of patients with cleidocranial dysplasia (Quack et al., 1999; Song et al., 1999; Zhou et al., 1999). R135G, G138D and R171G were described in MoAML, while R139Q was reported in FPD/AML and cleidocranial dysplasia (Quack et al., 1999; Song et al., 1999; Zhou et al., 1999; Preudhomme et al., 2000). Although G138 (strand βE′) does not interact directly with the DNA, the G138D mutation would be expected to disrupt the conformation of the βE′-F and βC-D loops, thus disrupting binding.

Several other mutations are described in the context of CCD: K167N, F146S, M124R and S140N (Lee et al., 1997; Werner et al., 1999; Zhang et al., 2000). Loss of DNA binding by K167N is explained by disruption of a critical nucleotide backbone contact (FIG. 8). The mutations L148F, F146S and M124R would be expected to destabilise the overall fold of the Runt domain, as they affect residues projecting into the hydrophobic core of the structure. The structural role of S140 was discussed above.

In summary, the structural and mutational analysis of the AML1 Runt domain-CBFβ-DNA ternary complex clearly explains the molecular consequences of the human disease-associated mutations on DNA binding.

Rel-like DNA Contacts from the Extended Runt Domain C-terminal ‘Tail’

The AML1 Runt domain belongs to the Ig domain superfamily of transcription factors with closest structural homology to STAT3β (Warren et al., 2000). In common with the other members of the superfamily, the Runt domain contacts DNA using loops from a single face of the β-barrel core (FIG. 7). Thus, the βA′-B loop corresponds structurally and functionally to the AB recognition loop of NF-κB, the T domain of Brachyury, the STATs and p53. The AB loops all make major groove interactions via arginine side chains, and the relative orientation of the domains with respect to DNA is maintained. Moreover, the Runt domain βE′-F loop is analogous structurally and functionally to the E≧F loop of NFAT (Chen et al., 1998) and the L3 loop of p53 (Cho et al., 1994), which bridge across the DNA nucleotide backbone from the major to the minor groove to make base contacts.

However, the Runt domain has a distinctive variation in its DNA binding architecture. Most of the base contacts in the major groove are made from an unusual extended C-terminal ‘tail’ projecting from the core Ig β-barrel. By contrast, the p53 DNA binding domain (Cho et al., 1994) and Brachyury T domain (Müller and Herrmann, 1997) use C-terminal α-helical extensions to contact the major and minor grooves, respectively. The functional importance of residues within the Runt domain ‘tail’ for DNA recognition is demonstrated by alanine mutagenesis and the mapping of human disease-associated mutations to this region. Use of an extended ‘tail’ in the major groove enhances the area of accessible surface (1519 Å²) buried on DNA binding. This structural variation may maximise the surface area available to the DNA-bound Runt domain-CBFβ complex for protein-protein interaction in the context of a higher order transcription complex. Thus, the observed distortion and bending of the DNA observed in the Runt domain-CBFβ-DNA ternary complex may facilitate interaction with factors such as CREB/ATF, LEF1 and Ets-1 on the T cell receptor enhancer (Giese et al., 1995). The ability of the C-terminus and the βE′-F loop of the Runt domain to adopt alternate conformations may play a role in regulating the interaction of the full length AML1 protein with DNA and with other proteins.

Despite lack of sequence homology, and the use of different structural elements for sequence-specific DNA binding, the structure of the Runt domain-CBFβ-DNA complex reveals remarkable conservation of the specific base contacts used with the class II Rel family of proteins (FIG. 11). This family is typified by NF-κB p65, RelB and c-Rel, the malarial protein Gambif1, and the Drosophila Dorsal and Dif proteins, which, like AML1, all contain transcription activation domains and have important roles in development (Cramer et al., 1999). Three guanine bases (G4, G6 and G7) are conserved in all Runt domain DNA binding recognition sites identified (Thomell et al., 1988; Kamachi et al., 1990; Wang and Speck, 1992). The Rel protein family has a core GG element that is contacted by three conserved residues, two arginines and a glutamate, from the AB recognition loop of a Rel monomer. By contrast, the Runt domain contributes two conserved arginines and an aspartate from the C-terminal ‘tail’ to contact the core GG element (FIGS. 2 and 5), and uses a single arginine from the βA′ strand to contact the third upstream G base. Thus, the Runt domain-CBFβ-DNA structure reveals clear conservation in the DNA binding mechanism with the Rel family of proteins and raises further questions about the evolutionary relationships between the Ig domain superfamily of transcription factors.

Structural and Functional Similarities to p53

The structure of the AML1 Runt domain-CBFβ-DNA ternary complex suggests that Runt domain loops βC-D and βE′-F are analogous structurally and functionally to p53 loops L2 and L3 (Cho et al., 1994). The structurally analogous residues K144 (Runt domain) and R249 (p53) make salt bridge interactions between the two loops. R249S is a common transforming mutation, and appears to reduce the DNA binding affinity of p53 by decreasing the stability of protein folding (DeDecker, 2000). An analogous Runt domain mutation, K144M, impairs DNA binding, while K144R does not (Crute et al., 1996). Runt domain N109 and p53 R175 (a mutation hotspot) make similar bridging interactions between the same two loops and mutation disrupts DNA binding function. (Cho et al., 1994; Akamatsu et al., 1997). As Runt domain loops βC-D and βE′-F and the analogous L2 and L3 loops in p53, have little regular secondary structure, the lack of extensive hydrogen bonding interactions may be compensated by side chain to side chain and side chain to backbone interactions between the loops. By contrast with the Runt domain-CBFβ complex, a bound zinc atom in addition to salt bridge interactions links the analogous loops in p53. Additional stabilisation of the conformation of Runt domain loops βC-D and βE′-F may be provided by interaction with CBFβ, which lowers the binding constant for the interaction of the Runt domain with DNA.

In summary, these comparisons suggest that changes in the conformation of Runt domain loops βC-D and βE′-F have an important influence in modulating the affinity of the interaction between the Runt domain-CBFβ complex and DNA.

Consistency with Biochemical Data

The base pair and nucleotide backbone contacts demonstrated in the crystal structure of the ternary complex (FIG. 8A) account for most of the observed effects of chemical modifications on the DNA. The three conserved guanine bases G4, G6 and G7 (strand E) in the core site were demonstrated to be critical contacts for the binding of Runt domain proteins from methylation interference analyses (Thornell et al., 1988; Kamachi et al., 1990; Wang and Speck, 1992). Base pairs T3, C5 and T8 (strand E) are not in direct contact with protein, in agreement with the sequence variability of the DNA in natural target sites at these positions. The selection for pyrimidine residues at these positions may reflect ‘indirect’ preferences for bases that allow bending of the DNA, supported by the high degree of roll and the inclination observed at these base steps in the structure. The observed distortion and bending of the DNA is consistent with circular permutation analysis of the AML1-CBFβ-DNA complex (Golling et al., 1996). The equilibrium binding constants for the Runt domain-CBFβ-DNA interaction is high (in the range of 10⁻¹⁰ to 10⁻¹² M), despite the fact that the Runt domain itself only binds DNA as a monomer. This may be explained by the large area of accessible surface (1519 Å²) that is buried upon complex formation, combined with the large number of direct base contacts observed in the structure (a total of 10).

These biochemical data are consistent with our structural and mutational analysis of the Runt domain-CBFβ-DNA ternary complex. All the critical side chain to DNA contacts identified in the structure are made by residues which are evolutionarily invariant in comparative sequence alignment of Runt domain proteins (Warren et al., 2000). This suggests that the DNA binding mechanism revealed by the AML1 Runt domain-CBFβ-DNA structure is general to the entire family of Runt domain proteins.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or biology or related fields are intended to be covered by the present invention. All publications mentioned in the present specification are herein incorporated by reference.

### REFERENCES

Akamatsu, Y., Ohno, T., Hirota, K., Kagoshima, H., Yodoi, J. and Shigesada, K. (1997a) Redox regulation of the DNA binding activity in transcription factor PEBP2. The roles of two conserved cysteine residues J Biol Chem 272, 14497-500.

Akamatsu, Y., Tsukumo, S., Kagoshima, H., Tsurushita, N. and Shigesada, K. (1997b) A simple screening for mutant DNA binding proteins: application to murine transcription factor PEBP2α subunit, a founding member of the Runt domain protein family Gene 185, 111-7.

Becker, S., Groner, B. and Müller, C. W. (1998) Three-dimensional structure of the Stat3β homodimer bound to DNA Nature 394, 145-51.

Berardi, M. J., Sun, C., Zehr, M., Abildgaard, F., Peng, J., Speck, N. A. and Bushweller, J. H. (1999) The Ig fold of the core binding factor α Runt domain is a member of a family of structurally and functionally related Ig-fold DNA-binding domains Structure Fold Des 7, 1247-56.

Bruhn, L., Munnerlyn, A. and Grosschedl, R. (1997) ALY, a context-dependent coactivator of LEF-1 and AML-1, is required for TCRα enhancer function Genes Dev 11, 640-53.

Brünger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T. and Warren, G. L. (1998) Crystallography & NMR system: A new software suite for macromolecular structure determination Acta Crystallogr D Biol Crystallogr 54, 905-21.

CCP4 (1994). Collaborative Computing Project 4: A suite of programs for protein crystallography. Acta Crystallogr D 50: 760-763.

Chen, L., Glover, J. N., Hogan, P. G., Rao, A. and Harrison, S. C. (1998) Structure of the DNA-binding domains from NFAT, Fos and Jun bound specifically to DNA Nature 392, 42-8. 392: 42-8.

Chen, Y. Q., Sengchanthalangsy, L. L., Hackett, A. and Ghosh, G. (2000). NF-kB p65 (RelA) homodimer uses distinct mechanisms to recognize DNA targets. Structure Fold Des 8: 419-28.

Cho, Y., Gorina, S., Jeffrey, P. D. and Pavletich, N. P. (1994). Crystal structure of a p53 tumor suppressor-DNA complex: understanding tumorigenic mutations [see comments]. Science 265: 346-55.

Cramer, P., Varrot, A., Barillas-Mury, C., Kafatos, F. C. and Müller, C. W. (1999). Structure of the specificity domain of the Dorsal homologue Gambifl bound to DNA. Structure Fold Des 7: 841-52.

Crute, B. E., Lewis, A. F., Wu, Z., Bushweller, J. H. and Speck, N. A. (1996) Biochemical and biophysical properties of the core-binding factor α2 (AML1) DNA-binding domain J Biol Chem 271, 26251-60.

DeDecker, B. S. (2000). Allosteric drugs: thinking outside the active-site box. Chem Biol 7: R103-7.

Esnouf, R. M. (1997) An extensively modified version of MolScript that includes greatly enhanced coloring capabilities J Mol Graph Model 15, 1324, 112-3.

Gamou, T., Kitamura, E., Hosoda, F., Shimizu, K., Shinohara, K., Hayashi, Y., Nagase, T., Yokoyama, Y. and Ohki, M. (1998). The partner gene of AML1 in t(16;21) myeloid malignancies is a novel member of the MTG8(ETO) family. Blood 91: 4028-37.

Gelmetti, V., Zhang, J., Fanelli, M., Minucci, S., Pelicci, P. G. and La7ar, M. A. (1998) Aberrant recruitment of the nuclear receptor corepressor-histone deacetylase complex by the acute myeloid leukaemia fusion partner ETO Mol Cell Biol 18, 7185-91.

Ghosh, G., van Duyne, G., Ghosh, S. and Sigler, P. B. (1995) Structure of NF-κB p50 homodimer bound to a κB site Nature 373, 303-10.

Giese, K., Kingsley, C., Kirshner, J. R. and Grosschedl, R. (1995) Assembly and function of a TCRα enhancer complex is dependent on LEF-1-induced DNA bending and multiple protein-protein interactions Genes Dev 9, 995-1008.

Goger, M., Gupta, V., Kim, W. Y., Shigesada, K., Ito, Y. and Werner, M. H. (1999) Molecular insights into PEBP2/CBFβ-SMMHC associated acute leukaemia revealed from the structure of PEBP2/CBFβ Nat Struct Biol 6, 620-3.

Golling, G., Li, L., Pepling, M., Stebbins, M. and Gergen, J. P. (1996) Drosophila homologs of the proto-oncogene product PEBP2/CBFβ regulate the DNA-binding properties of Runt Mol Cell Biol 16, 932-42.

Golub, T. R., Barker, G. F., Lovett, M. and Gilliland, D. G. (1994) Fusion of PDGF receptor β to a novel ets-like gene, tel, in chronic myelomonocytic leukaemia with t(5;12) chromosomal translocation Cell 77, 307-316.

Hendrickson, W. A., Horton, J. R. and LeMaster, D. M. (1990) Selenomethionyl proteins produced for analysis by multiwavelength anomalous diffraction (MAD): a vehicle for direct determination of three-dimensional structure EMBO J 9, 1665-72.

Hiebert, S. W., Sun, W., Davis, J. N., Golub, T., Shurtleff, S., Buijs, A., Downing, J. R., Grosveld, G., Roussell, M. F., Gilliland, D. G., Lenny, N. and Meyers, S. (1996) The t(12;21) translocation converts AML-1β from an activator to a repressor of transcription Mol Cell Biol 16, 1349-55.

Huang, X., Peng, J. W., Speck, N. A. and Bushweller, J. H. (1999) Solution structure of core binding factor α and map of the CBFβ binding site Nat Struct Biol 6, 624-7.

Imai, Y., Kurokawa, M., Izutsu, K., Hangaishi, A., Takeuchi, K., Maid, K., Ogawa, S., Chiba, S., Mitani, K. and Hirai, H. (2000). Mutations of the AML1 gene in myelodysplastic syndrome and their functional implications in leukemogenesis. Blood 96: 3154-60.

Jones, T. A., Zou, J. Y., Cowan, S. W. and Kjeldgaard, M. (1991) Improved methods for building protein models in electron density maps and the location of errors in these models Acta Crystallogr A 47, 110-9.

Kagoshima, H., Akamatsu, Y., Ito, Y. and Shigesada, K. (1996) Functional dissection of the α and α subunits of transcription factor PEBP2 and the redox susceptibility of its DNA binding activity J Biol Chem 271, 33074-82.

Kagoshima, H., Shigesada, K., Satake, M., Ito, Y., Miyoshi, H., Ohki, M., Pepling, M. and Gergen, P. (1993). The Runt domain identifies a new family of heteromeric transcriptional regulators [letter]. Trends Genet 9: 338-41.

Kamachi, Y., Ogawa, E., Asano, M., Ishida, S., Murakarni, Y., Satake, M., Ito, Y. and Shigesada, K. (1990) Purification of a mouse nuclear factor that binds to both the A and B cores of the polyomavirus enhancer J Virol 64, 4808-19.

Kanno, T., Kanno, Y., Chen, L. F., Ogawa, E., Kim, W. Y. and Ito, Y. (1998a) Intrinsic transcriptional activation-inhibition domains of the polyomavirus enhancer binding protein 2/core binding factor a subunit revealed in the presence of the β subunit Mol Cell Biol 18, 2444-54.

Kanno, Y., Kanno, T., Sakakura, C., Bae, S. C. and Ito, Y. (1998b) Cytoplasmic sequestration of the polyomavirus enhancer binding protein 2 (PEBP2)/core binding factor α (CBFβ) subunit by the leukaemia-related PEBP2/CBFβ-SMMHC fusion protein inhibits PEBP2/CBF-mediated transactivation Mol Cell Biol 18, 4252-61.

Komori, T., Yagi, H., Nomura, S., Yamaguchi, A., Sasaki, K., Deguchi, K., Shimizu, Y., Bronson, R. T., Gao, Y. H., Inada, M., Sato, M., Okamoto, R., Kitamura, Y., Yoshiki, S. and Kishimoto, T. (1997) Targeted disruption of Cbfal results in a complete lack of bone formation owing to maturational arrest of osteoblasts Cell 89, 755-64.

Kraulis, P. J. (1991) A program to produce both detailed and schematic plots of protein structures J Appl Crystallogr 24, 946-950.

Lee, B., Thirunavukkarasu, K., Zhou, L., Pastore, L., Baldini, A., Hecht, J., Geoffroy, V., Ducy, P. and Karsenty, G. (1997) Missense mutations abolishing DNA binding of the osteoblast-specific transcription factor OSF2/CBFA1 in cleidocranial dysplasia Nat Genet 16, 307-10.

Lenny, N., Meyers, S. and Hiebert, S. W. (1995) Functional domains of the t(8;21) fusion protein, AML-1/ETO Oncogene 11, 1761-9.

Leslie, A. G. W. (1992). Recent changes to the MOSFLM package for film and image plate data. Joint CCP4 and ESF-EACMB Newsletter on Protein Crystallography, Daresbury Laboratory, Warrington, UK,

Levanon, D., Negreanu, V., Bernstein, Y., Bar-Am, I., Avivi, L. and Groner, Y. (1994) AML1, AML2, and AML3, the human members of the runt domain gene-family: cDNA structure, expression, and chromosomal localization Genomics 23, 425-32.

Li, L. H. and Gergen, J. P. (1999) Differential interactions between Brother proteins and Runt domain proteins in the Drosophila embryo and eye Development 126, 3313-22.

Liu, P., Tarle, S. A., Claxton, D. F., Marlton, P., Freedman, M., Siciliano, M. J. and Collins, F. S. (1993) Fusion between transcription factor CBFβ/PEBP2β and a myosin heavy chain in acute myeloid leukaemia Science 261, 1041-1044.

Look, A. T. (1997) Oncogenic transcription factors in the human acute leukaemias Science 278, 1059-1065.

Lu, X. J., Shakked, Z. and Olson, W. K. (2000). A-form confornational motifs in ligand-bound DNA structures. J Mol Biol 300: 819-40.

Lutterbach, B., Hou, Y., Durst, K. L. and Hiebert, S. W. (1999) The inv(16) encodes an acute myeloid leukaemia 1 transcriptional corepressor Proc Natl Acad Sci USA 96, 12822-7.

Lutterbach, B., Westendorf, J. J., Linggi, B., Patten, A., Moniwa, M., Davie, J. R., Huynh, K. D., Bardwell, V. J., Lavinsky, R. M., Rosenfeld, M. G., Glass, C., Seto, E. and Hiebert, S. W. (1998) ETO, a target of t(8;21) in acute leukaemia, interacts with the N-CoR and mSin3 corepressors Mol Cell Biol 18, 7176-84.

Manley, N. R., O'Connell, M., Sun, W., Speck, N. A. and Hopkins, N. (1993) Two factors that bind to highly conserved sequences in mammalian type C retroviral enhancers J Virol 67, 1967-75.

Melnikova, I. N., Crute, B. E., Wang, S. and Speck, N. A. (1993) Sequence specificity of the core-binding factor J Virol 67, 2408-11.

Meyers, S., Downing, J. R. and Hiebert, S. W. (1993). Identification of AML-1 and the (8;21) translocation protein (AML-1/ETO) as sequence-specific DNA-binding proteins: the runt homology domain is required for DNA binding and protein-protein interactions. Mol Cell Biol 13: 633645.

Miroux, B. and Walker, J. E. (1996) Over-production of proteins in Escherichia coli: mutant hosts that allow synthesis of some membrane proteins and globular proteins at high levels J Mol Biol 260,289-98.

Miyoshi, H., Kozu, T., Shimizu, K., Enomoto, K., Maseki, N., Kaneko, Y., Kamada, N. and Ohki, M. (1993) The t(8;21) translocation in acute myeloid leukaemia results in production of an AML1-MTG8 fusion transcript EMBO J 12,2715-2721.

Miyoshi, H., Shimizu, K., Kozu, T., Maseki, N., Kaneko, Y. and Ohki, M. (1991) t(8;21) breakpoints on chromosome 21 in acute myeloid leukaemia are clustered within a limited region of a single gene, AML1 Proc. Natl. Acad. Sci. USA 88, 10431-10434.

Müller, C. W. and Herrmann, B. G. (1997). Crystallographic structure of the T domain-DNA complex of the Brachyury transcription factor. Nature 389: 884-8.

Mundlos, S., Otto, F., Mundlos, C., Mulliken, J. B., Aylsworth, A. S., Albright, S., Lindhout, D., Cole, W. G., Henn, W., Knoll, J. H., Owen, M. J., Mertelsmann, R, Zabel, B. U. and Olsen, B. R. (1997) Mutations involving the transcription factor CBFA1 cause cleidocranial dysplasia Cell 89, 773-9.

Murzin, A. G., Brenner, S. E., Hubbard, T. and Chothia, C. (1995) SCOP: a structural classification of proteins database for the investigation of sequences and structures J Mol Biol 247, 536-40.

Nagata, T., Gupta, V., Sorce, D., Kim, W. Y., Sali, A., Chait, B. T., Shigesada, K., Ito, Y. and Werner, M. H. (1999) Immunoglobulin motif DNA recognition and heterodimerization of the PEBP2/CBF Runt domain Nat Struct Biol 6, 615-9.

Nekludova, L. and Pabo, C. O. (1994). Distinctive DNA conformation with enlarged major groove is found in Zn- finger-DNA and other protein-DNA complexes. Proc Natl Acad Sci USA 91: 6948-52.

Nicholls, A., Sharp, K. A. and Honig, B. (1991) Protein folding and association: insights from the interfacial and thermodynamic properties of hydrocarbons Proteins 11, 281-96.

North, T., Gu, T. L., Stacy, T., Wang, Q., Howard, L., Binder, M., Marín-Padilla, M. and Speck, N. A. (1999) Cbfa2 is required for the formation of intra-aortic hematopoietic clusters Development 126, 2563-75.

Nucifora, G., Begy, C. R., Erickson, P., Drabkin, H. A. and Rowley, J. D. (1993) The 3;21 translocation in myelodysplasia results in a fusion transcript between the AML1 gene and the gene for EAP, a highly conserved protein associated with the Epstein-Barr virus small RNA EBER1 Proc. Natl. Acad. Sci. USA 90, 7784-7788.

Ogawa, E., Inuzuka, M., Maruyama, M., Satake, M., Naito-Fujimoto, M., Ito, Y. and Shigesada, K. (1993a) Molecular cloning and characterization of PEBP2β, the heterodimeric partner of a novel Drosophila runt-related DNA binding protein PEBP2 α Virology 194, 314-31.

Ogawa, E., Maruyama, M., Kagoshima, H., Inuzuka, M., Lu, J., Satake, M., Shigesada, K. and Ito, Y. (1993b) PEBP2/PEA2 represents a family of transcription factors homologous to the products of the Drosophila runt gene and the human AML1 gene Proc Natl Acad Sci USA 90, 6859-63.

Okuda, T., van Deursen, J., Hiebert, S. W., Grosveld, G. and Downing, J. R. (1996) AML1, the target of multiple chromosomal translocations in human leukaemia, is essential for normal fetal liver hematopoiesis Cell 84, 321-30.

Osato, M., Asou, N., Abdalla, E., Hoshino, K., Yamasaki, H., Okubo, T., Suzushima, H., Takatsuki, K., Kanno, T., Shigesada, K. and Ito, Y. (1999) Biallelic and heterozygous point mutations in the Runt domain of the AML1/PEBP2αB gene associated with myeloblastic leukaemias Blood 93, 1817-24.

Otto, F., Thornell, A. P., Crompton, T., Denzel, A., Gilmour, K. C., Rosewell, I. R., Stamp, G. W., Beddington, R. S., Mundlos, S., Olsen, B. R., Selby, P. B. and Owen, M. J. (1997) Cbfa1, a candidate gene for cleidocranial dysplasia syndrome, is essential for osteoblast differentiation and bone development Cell 89, 765-71.

Petrovick, M. S., Hiebert, S. W., Friedman, A. D., Hetherington, C. J., Tenen, D. G. and Zhang, D. E. (1998) Multiple functional domains of AML1: PU.1 and C/EBPα synergize with different regions of AML1 Mol Cell Biol 18, 3915-25.

Preudhomme, C., Warot-Loze, D., Roumier, C., Grardel-Duflos, N., Garand, R, Lai, J. L., Dastugue, N., Macintyre, E., Denis, C., Bauters, F., Kerckaert, J. P., Cosson, A. and Fenaux, P. (2000). High incidence of biallelic point mutations in the runt domain of the AML1/PEBP2αB gene in Mo acute myeloid leukaemia and in myeloid malignancies with acquired trisomy 21. Blood 96: 2862-9.

Quack, I., Vonderstrass, B., Stock, M., Aylsworth, A. S., Becker, A., Brueton, L., Lee, P. J., Majewski, F., Mulliken, J. B., Suri, M., Zenker, M., Mundlos, S. and Otto, F. (1999) Mutation analysis of core binding factor A1 in patients with cleidocranial dysplasia Am J Hum Genet 65, 1268-78.

Rabbitts, T. H. (1994) Chromosomal translocations in human cancer Nature 372, 143-149.

Ramakrishnan, V. and Biou, V. (1997) Treatment of multiwavelength anomalous diffraction data as a special case of multiple isomorphous replacement Methods Enzymol 276, 538-57.

Rodan, G. A. and Harada, S. (1997) The missing bone Cell 89, 677-80.

Romana, S. P., Mauchauffe, M., Le Coniat, M., Chumakov, I., Le Paslier, D., Berger, R. and Bernard, O. A. (1995) The t(12;21) of acute lymphoblastic leukaemia results in a tel-AML1gene fusion Blood 85, 3662-70.

Rubnitz, J. E. and Look, A. T. (1998). Molecular basis of leukemogenesis. Curr Opin Hematol 5: 264-70.

Sasaki, K., Yagi, H., Bronson, R. T., Tominaga, K., Matsunashi, T., Deguchi, K., Tani, Y., Kishimoto, T. and Komori, T. (1996) Absence of fetal liver hematopoiesis in mice deficient in transcriptional coactivator core binding factor β Proc Natl Acad Sci USA 93, 12359-63.

Schiestl, R. H. and Gietz, R. D. (1989). High efficiency transformation of intact yeast cells using single stranded nucleic acids as a carrier. Curr Genet 16: 33946.

Shurtleff, S. A., Meyers, S., Hiebert, S. W., Raimondi, S. C., Head, D. R., Willman, C. L., Wolman, S., Slovak, M. L., Carroll, A. J., Behm, F. and et al. (1995). Heterogeneity in CBFb/MYH11 fusion messages encoded by the inv(16)(p13q22) and the t(16;16)(p13;q22) in acute myelogenous leukaemia. Blood 85: 3695-703.

Song, W. J., et al., (1999) Haploinsufficiency of CBFA2 causes familial thrombocytopenia with propensity to develop acute myelogenous leukaemia Nat Genet 23, 166-75.

Tang, Y. Y., Crute, B. E., Kelley, J. J., Huang, X., Yan, J., Shi, J., Hartman, K. L., Laue, T. M., Speck, N. A. and Bushweller, J. H. (2000a). Biophysical characterization of interactions between the core binding factor a and b subunits and DNA. FEBS Lett 470: 167-72.

Tang, Y. Y., Shi, J., Zhang, L., Davis, A., Bravo, J., Warren, A. J., Speck, N. A. and Bushweller, J. H. (2000b). Energetic and functional contribution of residues in the core binding factor b (CBFb) subunit to heterodimerization with CBFa. J Biol Chem 275: 39579-88.

Terwilliger, T. C. and Berendzen, J. (1999) Automated MAD and MIR structure solution Acta Crystallogr D Biol Crystallogr 55, 849-61.

Thompson, J. D., Higgins, D. G. and Gibson, T. J. (1994) CLUSTAL W: improving the sensitivity of progressive multiple sequence alignment through sequence weighting, position-specific gap penalties and weight matrix choice Nucleic Acids Res 22, 4673-80.

Thomell, A., Hallberg, B. and Grundström, T. (1988) Differential protein binding in lymphocytes to a sequence in the enhancer of the mouse retrovirus SL3-3 Mol Cell Biol 8, 1625-37.

Thornell, A., Hallberg, B. and Grundström, T. (1991) Binding of SL3-3 enhancer factor 1 transcriptional activators to viral and chromosomal enhancer sequences J Virol 65, 42-50.

Wang, Q., Stacy, T., Binder, M., Marín-Padilla, M., Sharpe, A. H. and Speck, N. A. (1996a) Disruption of the Cbfa2 gene causes necrosis and hemorrhaging in the central nervous system and blocks definitive hematopoiesis Proc Natl Acad Sci USA 93, 3444-9.

Wang, Q., Stacy, T., Miller, J. D., Lewis, A. F., Gu, T. L., Huang, X., Bushweller, J. H., Bories, J. C., Alt, F. W., Ryan, G., Liu, P. P., Wynshaw-Boris, A., Binder, M., Marín-Padilla, M., Sharpe, A. H. and Speck, N. A. (1996b) The CBFβ subunit is essential for CBFα2 (AML1) function in vivo Cell 87, 697-708.

Wang, S. W. and Speck, N. A. (1992) Purification of core-binding factor, a protein that binds the conserved core site in murine leukaemia virus enhancers. Mol Cell Biol 12: 89-102.

Wang, S., Wang, Q., Crute, B. E., Melnikova, I. N., Keller, S. and Speck, N. A. (1993) Cloning and characterization of subunits of the T cell receptor and murine leukaemia virus enhancer core-binding factor Mol. Cell. Biol. 13, 3324-3339.

Warren, A. J., Bravo, J., Williams, R. L. and Rabbitts, T. H. (2000). Structural basis for the heterodimeric interaction between the acute leukaemia-associated transcription factors AML1 and CBFb. Embo J 19: 3004-15.

Werner, M. H., Shigesada, K. and Ito, Y. (1999). Runt domains take the lead in hematopoiesis and osteogenesis [news]. Nat Med 5: 1356-7.

Wotton, D., Ghysdael, J., Wang, S., Speck, N. A. and Owen, M. J. (1994) Cooperative binding of Ets-1 and core binding factor to DNA Mol Cell Biol 14, 840-50.

Zhang, Y. W., Yasui, N., Kakazu, N., Abe, T., Takada, K., Imai, S., Sato, M., Nomura, S., Ochi, T., Okuzumi, S., Nogami, H., Nagai, T., Ohashi, H. and Ito, Y. (2000). PEBP2aA/CBFA1 mutations in Japanese cleidocranial dysplasia patients. Gene 244: 21-8.

Zhou, G., Chen, Y., Zhou, L., Thirunavukkarasu, K., Hecht, J., Chitayat, D., Gelb, B. D., Pirinen, S., Berry, S. A., Greenberg, C. R., Karsenty, G. and Lee, B. (1999) CBFA1 mutation analysis and functional correlation with phenotypic variability in cleidocranial dysplasia Hum Mol Genet 8, 2311-6.

Zhou, P., Sun, L. J., Dötsch, V., Wagner, G. and Verdine, G. L. (1998) Solution structure of the core NFATC1/DNA complex Cell 92, 687-96.

### SEQUENCES

### SEQ ID NO: 1

AML1 (residues 50-178)

SMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRR

### SEQ ID NO: 2

CBFβ (Residues 1-135)

MPRVVPDQRSKFENEEFFRKLSRECEIKYTGFRDRPHEERQARFQNACRDGRSEIAFVATGTNLSLQFFPASWQGEQRQTPSREYVDLEREAGKVYLKAPMILNGVCVIWKGWIDLQRLDGMGCLEFDEERAQQE

TABLE 1
Data collection, structure determination and refinement statistics
Data collection and MIR phasing statistics

| | Resolution | Observations/ | Completness | R_{merge}¹ |
|---|---|---|---|---|
| Data set | (Å) | unique reflections | (last shell) % | (last shell) |
| Se-Met λ1 | 3.2 | 78580/60509 | 88.9(84.1) | 0.047(0.15) |
| Se-Met λ2 | 3.2 | 86114/63424 | 92.8(85.4) | 0.066(0.25) |
| Native | 2.6 | 259515/71198 | 99.3(99.2) | 0.084(0.28) |

Structure refinement statistics

| Resolution | Protein | | | R_{free}² | r.m.s.d. from ideality³ | | |
|---|---|---|---|---|---|---|---|
| (Å) | atoms | Waters | R_{cryst}² | (% data) | bonds | angles | dihedral |
| 25.0-2.6 | 9551 | 121 | 26.46 | 29.49 | 0.0072 | 1.3921 | 24.76 |
| | | | | used)³ | | | |

¹R_{merge} = ΣhklΣi|Iᵢ(hkl) − <I(hkl)>|/ΣhklΣi Iᵢ(hkl)
²R_{cryst} and R_{free} = Σ|F_{obs} − F_{calc}|/ΣF_{obs}; R_{free} calculated with the percentage of the data shown in parentheses
³r.m.s. deviations for bond angles and lengths in regard to Engh & Huber parameters.

TABLE 2
Interactions between the AML1 Runt domain and CBFb

| | |
|---|---|
| Runt domain | CBFb |

| Residue | Location | Residue | Location |
|---|---|---|---|
| Asp 66 | loop bO-A | Lys 11 | helix H1 |
| Asp 66 | loop bO-A | Asn 104 | loop b4-b5 |
| Ser 67 | loop bO-A | Asn 104 | loop b4-b5 |
| Pro68* | loop bO-A | Arg 3 | N-terminal loop |
| Pro68 | loop bO-A | Val 4 | N-terminal loop |
| Pro68 | loop bO-A | Val 5 | N-terminal loop |
| Pro68 | loop bO-A | Asn 104 | loop b4-b5 |
| Pro68 | loop bO-A | Gly 105 | loop b4-b5 |
| Asn 69* | loop bO-A | Arg 3 | N-terminal loop |
| Leu 94* | loop bB-C | Arg 3 | N-terminal loop |
| Met 106 | strand bC | Glu 54 | strand b2 |
| Met 106 | strand bC | Asn 63 | strand b3 |
| Met 106 | strand bC | Leu 64 | strand b3 |
| Met 106 | strand bC | Ser 65 | strand b3 |
| Asn 109 | strand bC | Gly 61 | loop b2-b3 |
| Tyr 113 | loop bC-D | Lys 28 | strand b1 |
| Tyr 113 | loop bC-D | Thr 30 | strand b1 |
| Tyr 113 | loop bC-D | Arg 33 | loop b1-helix H3 |
| Tyr 113* | loop bC-D | Val 58 | strand b2 |
| Tyr 113 | loop bC-D | Asn 63 | strand b3 |
| Ser 114 | strand bD | Thr 30 | strand b1 |
| Ser 114* | strand bD | Asn 63 | strand b3 |
| Thr 149* | strand bF | Asn 63 | strand b3 |
| Phe 153 | loop bF-G | Ser 65 | strand b3 |
| Phe 153 | loop bF-G | Gln 67 | strand b3 |
| CPR 156 | loop bF-G | Pro 100 | strand b4 |
| CPR 156 | loop bF-G | Met 101 | strand b4 |
| Pro 157 | loop bF-G | Gln 67 | strand b3 |
| Pro 157 | loop bF-G | Pro 100 | strand b4 |
| Pro 157 | loop bF-G | Met 101 | strand b4 |
| Pro 157* | loop bF-G | Ile 102 | strand b4 |
| Gln 158 | strand bG | Pro 2 | N-terminal loop |
| Gln 158* | strand bG | Arg 3 | N-terminal loop |
| Gln 158 | strand bG | Ile 102 | strand b4 |
| Val 159 | strand bG | Leu 64 | strand b3 |
| Val 159 | strand bG | Met 101 | strand b4 |
| Val 159* | strand bG | Ile 102 | strand b4 |
| Val 159 | strand bG | Leu 103 | loop b4-b5 |
| Val 159* | strand bG | Asn 104 | loop b4-b5 |
| Ala 160 | strand bG | Asn 104 | loop b4-b5 |
| Thr 161* | strand bG | Asn 104 | loop b4-b5 |
| His 163 | strand bG | Phe 17 | helix H2 |

*Indicates potential hydrogen bonding interactions. Pairwise interactions were identified in CNS REF

TABLE 3
REMARK coordinates from restrained individual B-factor refinement
REMARK refinement resolution: 500.0-2.6 A
REMARK starting r = 0.2677 free_r = 0.2973
REMARK final r = 0.2646 free_r = 0.2949
REMARK B rmsd for bonded mainchain atoms = 1.992 target = 1.5
REMARK B rmsd for bonded sidechain atoms = 3.077 target = 2.0
REMARK B rmsd for angle mainchain atoms = 3.299 target = 2.0
REMARK B rmsd for angle sidechain atoms = 4.527 target = 2.5
REMARK wa = 2.84177
REMARK rweight = 5.800572E−02
REMARK target = mlf steps = 60
REMARK sg = P2 (1) a = 103.259 b = 79.383 c = 130.101 alpha = 90
beta = 101.388 gamma = 90
REMARK parameter file 1: CNS_TOPPAR: protein_rep.param
REMARK parameter file 2: CNS_TOPPAR: water_rep.param
REMARK molecular structure file: aml.mtf
REMARK input coordinates: aml_minimize.pdb
REMARK reflection file = gw10_2.6_free.hkl
REMARK ncs = none
REMARK B-correction resolution: 6.0-2.6
REMARK initial B-factor correction applied to fobs:
REMARK B11 = 8.510 B22 = −4.214 B33 = −4.296
REMARK B12 = 0.000 B13 = −1.074 B23 = 0.000
REMARK B-factor correction applied to coordinate array B: −0.259
REMARK bulk solvent: density level = 0.354179 e/A\hat{ }3, B-factor = 41.3786 A\hat{ }2
REMARK reflections with |Fobs|/sigma_F < 0.0 rejected
REMARK reflections with |Fobs| > 10000 * rms (Fobs) rejected
REMARK theoretical total number of refl. in resol. range: 63790 (100.0%)
REMARK number of unobserved reflections (no entry or |F|=0): 467 (0.7%)
REMARK number of reflections rejected: 0 (0.0%)
REMARK total number of reflections used: 63323 (99.3%)
REMARK number of reflections in working set: 61418 (96.3%)
REMARK number of reflections in test set: 1905 (3.0%)
CRYST1 103.259 79.383 130.101 90.00 101.39 90.00 P 21
REMARK
FILENAME = “/pnac1/jero/ajw/refi/cns/A2R/A2R_edited/am1_bindividual.pdb”
REMARK DATE: 10-Nov-99 13:15:23 created by user: root
REMARK VERSION: 0.9a

| | | | | | | | | | | | |
|---|---|---|---|---|---|---|---|---|---|---|---|
| ATOM | 1950 | CB | ALA | C | 54 | −9.503 | 20.880 | 39.829 | 1.00 | 96.49 | C |
| ATOM | 1951 | C | ALA | C | 54 | −10.892 | 22.616 | 38.661 | 1.00 | 96.43 | C |
| ATOM | 1952 | O | ALA | C | 54 | −10.702 | 23.768 | 39.052 | 1.00 | 97.28 | C |
| ATOM | 1953 | N | ALA | C | 54 | −8.455 | 22.357 | 38.148 | 1.00 | 96.12 | C |
| ATOM | 1954 | CA | ALA | C | 54 | −9.714 | 21.643 | 38.522 | 1.00 | 96.83 | C |
| ATOM | 1955 | N | ALA | C | 55 | −12.097 | 22.139 | 38.340 | 1.00 | 95.69 | C |
| ATOM | 1956 | CA | ALA | C | 55 | −13.332 | 22.935 | 38.405 | 1.00 | 95.09 | C |
| ATOM | 1957 | CB | ALA | C | 55 | −14.501 | 22.042 | 38.834 | 1.00 | 93.93 | C |
| ATOM | 1958 | C | ALA | C | 55 | −13.248 | 24.157 | 39.322 | 1.00 | 94.44 | C |
| ATOM | 1959 | O | ALA | C | 55 | −13.473 | 25.291 | 38.884 | 1.00 | 93.92 | C |
| ATOM | 1960 | N | ALA | C | 56 | −12.935 | 23.915 | 40.594 | 1.00 | 94.26 | C |
| ATOM | 1961 | CA | ALA | C | 56 | −12.800 | 24.979 | 41.588 | 1.00 | 93.06 | C |
| ATOM | 1962 | CB | ALA | C | 56 | −11.815 | 26.042 | 41.080 | 1.00 | 92.96 | C |
| ATOM | 1963 | C | ALA | C | 56 | −14.142 | 25.625 | 41.955 | 1.00 | 91.87 | C |
| ATOM | 1964 | O | ALA | C | 56 | −14.760 | 26.312 | 41.134 | 1.00 | 92.62 | C |
| ATOM | 1965 | N | ALA | C | 57 | −14.568 | 25.396 | 43.200 | 1.00 | 88.52 | C |
| ATOM | 1966 | CA | ALA | C | 57 | −15.824 | 25.915 | 43.753 | 1.00 | 84.71 | C |
| ATOM | 1967 | CB | ALA | C | 57 | −16.975 | 25.711 | 42.758 | 1.00 | 84.53 | C |
| ATOM | 1968 | C | ALA | C | 57 | −16.107 | 25.136 | 45.038 | 1.00 | 82.38 | C |
| ATOM | 1969 | O | ALA | C | 57 | −15.191 | 24.531 | 45.601 | 1.00 | 82.34 | C |
| ATOM | 1970 | N | ALA | C | 58 | −17.365 | 25.171 | 45.494 | 1.00 | 77.91 | C |
| ATOM | 1971 | CA | ALA | C | 58 | −17.833 | 24.447 | 46.688 | 1.00 | 73.46 | C |
| ATOM | 1972 | CB | ALA | C | 58 | −16.965 | 23.195 | 46.937 | 1.00 | 72.77 | C |
| ATOM | 1973 | C | ALA | C | 58 | −18.036 | 25.199 | 48.014 | 1.00 | 70.73 | C |
| ATOM | 1974 | O | ALA | C | 58 | −18.837 | 24.761 | 48.839 | 1.00 | 72.41 | C |
| ATOM | 1975 | N | PRO | C | 59 | −17.310 | 26.309 | 48.261 | 1.00 | 66.88 | C |
| ATOM | 1976 | CD | PRO | C | 59 | −16.111 | 26.872 | 47.619 | 1.00 | 64.59 | C |
| ATOM | 1977 | CA | PRO | C | 59 | −17.568 | 26.974 | 49.548 | 1.00 | 63.39 | C |
| ATOM | 1978 | CB | PRO | C | 59 | −16.550 | 28.104 | 49.563 | 1.00 | 62.51 | C |
| ATOM | 1979 | CG | PRO | C | 59 | −15.415 | 27.528 | 48.789 | 1.00 | 65.04 | C |
| ATOM | 1980 | C | PRO | C | 59 | −19.012 | 27.483 | 49.604 | 1.00 | 61.79 | C |
| ATOM | 1981 | O | PRO | C | 59 | −19.700 | 27.320 | 50.610 | 1.00 | 60.35 | C |
| ATOM | 1982 | N | GLY | C | 60 | −19.454 | 28.103 | 48.510 | 1.00 | 60.66 | C |
| ATOM | 1983 | CA | GLY | C | 60 | −20.818 | 28.602 | 48.407 | 1.00 | 58.68 | C |
| ATOM | 1984 | C | GLY | C | 60 | −21.158 | 29.976 | 48.969 | 1.00 | 57.35 | C |
| ATOM | 1985 | O | GLY | C | 60 | −20.308 | 30.866 | 49.070 | 1.00 | 57.56 | C |
| ATOM | 1986 | N | GLU | C | 61 | −22.434 | 30.129 | 49.321 | 1.00 | 54.18 | C |
| ATOM | 1987 | CA | GLU | C | 61 | −22.992 | 31.354 | 49.888 | 1.00 | 50.07 | C |
| ATOM | 1988 | CB | GLU | C | 61 | −24.508 | 31.224 | 49.890 | 1.00 | 50.41 | C |
| ATOM | 1989 | CG | GLU | C | 61 | −25.214 | 32.363 | 50.547 | 1.00 | 57.12 | C |
| ATOM | 1990 | CD | GLU | C | 61 | −25.222 | 33.589 | 49.689 | 1.00 | 59.19 | C |
| ATOM | 1991 | OE1 | GLU | C | 61 | −24.164 | 33.910 | 49.115 | 1.00 | 60.92 | C |
| ATOM | 1992 | OE2 | GLU | C | 61 | −26.288 | 34.232 | 49.595 | 1.00 | 63.49 | C |
| ATOM | 1993 | C | GLU | C | 61 | −22.477 | 31.569 | 51.324 | 1.00 | 47.40 | C |
| ATOM | 1994 | O | GLU | C | 61 | −22.909 | 30.885 | 52.260 | 1.00 | 48.01 | C |
| ATOM | 1995 | N | LEU | C | 62 | −21.569 | 32.521 | 51.511 | 1.00 | 40.37 | C |
| ATOM | 1996 | CA | LEU | C | 62 | −21.004 | 32.746 | 52.845 | 1.00 | 36.36 | C |
| ATOM | 1997 | CB | LEU | C | 62 | −19.508 | 32.411 | 52.815 | 1.00 | 29.31 | C |
| ATOM | 1998 | CG | LEU | C | 62 | −19.182 | 30.978 | 52.368 | 1.00 | 28.80 | C |
| ATOM | 1999 | CD1 | LEU | C | 62 | −17.706 | 30.859 | 52.066 | 1.00 | 26.49 | C |
| ATOM | 2000 | CD2 | LEU | C | 62 | −19.601 | 29.974 | 53.443 | 1.00 | 22.10 | C |
| ATOM | 2001 | C | LEU | C | 62 | −21.199 | 34.128 | 53.471 | 1.00 | 33.73 | C |
| ATOM | 2002 | O | LEU | C | 62 | −21.354 | 35.132 | 52.772 | 1.00 | 36.23 | C |
| ATOM | 2003 | N | VAL | C | 63 | −21.206 | 34.160 | 54.798 | 1.00 | 29.49 | C |
| ATOM | 2004 | CA | VAL | C | 63 | −21.323 | 35.408 | 55.551 | 1.00 | 31.48 | C |
| ATOM | 2005 | CB | VAL | C | 63 | −22.644 | 35.508 | 56.387 | 1.00 | 31.06 | C |
| ATOM | 2006 | CG1 | VAL | C | 63 | −23.828 | 35.619 | 55.486 | 1.00 | 30.92 | C |
| ATOM | 2007 | CG2 | VAL | C | 63 | −22.787 | 34.291 | 57.317 | 1.00 | 30.01 | C |
| ATOM | 2008 | C | VAL | C | 63 | −20.166 | 35.435 | 56.548 | 1.00 | 33.15 | C |
| ATOM | 2009 | O | VAL | C | 63 | −19.537 | 34.409 | 56.807 | 1.00 | 35.16 | C |
| ATOM | 2010 | N | ARG | C | 64 | −19.887 | 36.601 | 57.118 | 1.00 | 34.30 | C |
| ATOM | 2011 | CA | ARG | C | 64 | −18.817 | 36.702 | 58.100 | 1.00 | 35.37 | C |
| ATOM | 2012 | CB | ARG | C | 64 | −18.244 | 38.134 | 58.152 | 1.00 | 30.36 | C |
| ATOM | 2013 | CG | ARG | C | 64 | −18.051 | 38.753 | 56.768 | 1.00 | 38.22 | C |
| ATOM | 2014 | CD | ARG | C | 64 | −16.918 | 39.775 | 56.697 | 1.00 | 34.48 | C |
| ATOM | 2015 | NE | ARG | C | 64 | −15.676 | 39.139 | 57.116 | 1.00 | 35.82 | C |
| ATOM | 2016 | CZ | ARG | C | 64 | −14.461 | 39.499 | 56.724 | 1.00 | 30.77 | C |
| ATOM | 2017 | NH1 | ARG | C | 64 | −14.294 | 40.519 | 55.876 | 1.00 | 23.82 | C |
| ATOM | 2018 | NH2 | ARG | C | 64 | −13.418 | 38.816 | 57.181 | 1.00 | 20.56 | C |
| ATOM | 2019 | C | ARG | C | 64 | −19.423 | 36.331 | 59.448 | 1.00 | 35.66 | C |
| ATOM | 2020 | O | ARG | C | 64 | −20.615 | 36.529 | 59.669 | 1.00 | 37.90 | C |
| ATOM | 2021 | N | THR | C | 65 | −18.610 | 35.760 | 60.329 | 1.00 | 35.18 | C |
| ATOM | 2022 | CA | THR | C | 65 | −19.055 | 35.398 | 61.667 | 1.00 | 33.06 | C |
| ATOM | 2023 | CB | THR | C | 65 | −18.449 | 34.048 | 62.081 | 1.00 | 32.31 | C |
| ATOM | 2024 | OG1 | THR | C | 65 | −17.029 | 34.175 | 62.197 | 1.00 | 29.11 | C |
| ATOM | 2025 | CG2 | THR | C | 65 | −18.747 | 32.991 | 61.029 | 1.00 | 32.01 | C |
| ATOM | 2026 | C | THR | C | 65 | −18.506 | 36.528 | 62.543 | 1.00 | 32.60 | C |
| ATOM | 2027 | O | THR | C | 65 | −17.857 | 37.420 | 62.027 | 1.00 | 29.94 | C |
| ATOM | 2028 | N | ASP | C | 66 | −18.743 | 36.508 | 63.851 | 1.00 | 34.44 | C |
| ATOM | 2029 | CA | ASP | C | 66 | −18.219 | 37.581 | 64.695 | 1.00 | 31.84 | C |
| ATOM | 2030 | CB | ASP | C | 66 | −18.914 | 37.597 | 66.045 | 1.00 | 32.52 | C |
| ATOM | 2031 | CG | ASP | C | 66 | −20.400 | 37.791 | 65.912 | 1.00 | 41.70 | C |
| ATOM | 2032 | OD1 | ASP | C | 66 | −20.785 | 38.582 | 65.028 | 1.00 | 42.68 | C |
| ATOM | 2033 | OD2 | ASP | C | 66 | −21.180 | 37.167 | 66.674 | 1.00 | 41.52 | C |
| ATOM | 2034 | C | ASP | C | 66 | −16.716 | 37.453 | 64.883 | 1.00 | 34.39 | C |
| ATOM | 2035 | O | ASP | C | 66 | −16.077 | 38.290 | 65.528 | 1.00 | 35.61 | C |
| ATOM | 2036 | N | SER | C | 67 | −16.146 | 36.398 | 64.312 | 1.00 | 34.17 | C |
| ATOM | 2037 | CA | SER | C | 67 | −14.709 | 36.180 | 64.397 | 1.00 | 32.58 | C |
| ATOM | 2038 | CB | SER | C | 67 | −14.393 | 34.716 | 64.696 | 1.00 | 31.68 | C |
| ATOM | 2039 | OG | SER | C | 67 | −13.012 | 34.460 | 64.504 | 1.00 | 29.20 | C |
| ATOM | 2040 | C | SER | C | 67 | −14.068 | 36.567 | 63.071 | 1.00 | 31.02 | C |
| ATOM | 2041 | O | SER | C | 67 | −14.589 | 36.268 | 62.001 | 1.00 | 35.75 | C |
| ATOM | 2042 | N | PRO | C | 68 | −12.928 | 37.249 | 63.128 | 1.00 | 27.83 | C |
| ATOM | 2043 | CD | PRO | C | 68 | −12.257 | 37.794 | 64.324 | 1.00 | 21.54 | C |
| ATOM | 2044 | CA | PRO | C | 68 | −12.251 | 37.658 | 61.890 | 1.00 | 26.94 | C |
| ATOM | 2045 | CB | PRO | C | 68 | −11.248 | 38.700 | 62.387 | 1.00 | 20.48 | C |
| ATOM | 2046 | CG | PRO | C | 68 | −10.903 | 38.166 | 63.790 | 1.00 | 18.95 | C |
| ATOM | 2047 | C | PRO | C | 68 | −11.562 | 36.493 | 61.154 | 1.00 | 28.28 | C |
| ATOM | 2048 | O | PRO | C | 68 | −11.026 | 36.674 | 60.056 | 1.00 | 27.05 | C |
| ATOM | 2049 | N | ASN | C | 69 | −11.581 | 35.305 | 61.759 | 1.00 | 27.29 | C |
| ATOM | 2050 | CA | ASN | C | 69 | −10.919 | 34.140 | 61.166 | 1.00 | 26.80 | C |
| ATOM | 2051 | CB | ASN | C | 69 | −10.081 | 33.436 | 62.228 | 1.00 | 24.12 | C |
| ATOM | 2052 | CG | ASN | C | 69 | −8.900 | 32.682 | 61.654 | 1.00 | 24.64 | C |
| ATOM | 2053 | OD1 | ASN | C | 69 | −8.329 | 33.072 | 60.636 | 1.00 | 22.53 | C |
| ATOM | 2054 | ND2 | ASN | C | 69 | −8.495 | 32.614 | 62.337 | 1.00 | 26.99 | C |
| ATOM | 2055 | C | ASN | C | 69 | −11.873 | 33.128 | 60.552 | 1.00 | 27.07 | C |
| ATOM | 2056 | O | ASN | C | 69 | −11.429 | 32.148 | 59.967 | 1.00 | 30.84 | C |
| ATOM | 2057 | N | PHE | C | 70 | −13.177 | 33.358 | 60.675 | 1.00 | 27.80 | C |
| ATOM | 2058 | CA | PHE | C | 70 | −14.142 | 32.412 | 60.126 | 1.00 | 26.17 | C |
| ATOM | 2059 | CB | PHE | C | 70 | −14.651 | 31.480 | 61.223 | 1.00 | 24.38 | C |
| ATOM | 2060 | CG | PHE | C | 70 | −13.565 | 30.794 | 61.966 | 1.00 | 27.28 | C |
| ATOM | 2061 | CD1 | PHE | C | 70 | −13.094 | 31.312 | 63.171 | 1.00 | 25.12 | C |
| ATOM | 2062 | CD2 | PHE | C | 70 | −12.951 | 29.673 | 61.431 | 1.00 | 22.24 | C |
| ATOM | 2063 | CE1 | PHE | C | 70 | −12.024 | 30.725 | 63.824 | 1.00 | 24.21 | C |
| ATOM | 2064 | CE2 | PHE | C | 70 | −11.879 | 29.085 | 62.083 | 1.00 | 27.66 | C |
| ATOM | 2065 | CZ | PHE | C | 70 | −11.414 | 29.612 | 63.278 | 1.00 | 24.67 | C |
| ATOM | 2066 | C | PHE | C | 70 | −15.347 | 32.988 | 59.417 | 1.00 | 27.68 | C |
| ATOM | 2067 | O | PHE | C | 70 | −15.908 | 34.008 | 59.820 | 1.00 | 30.11 | C |
| ATOM | 2068 | N | LEU | C | 71 | −15.731 | 32.306 | 58.347 | 1.00 | 25.86 | C |
| ATOM | 2069 | CA | LEU | C | 71 | −16.901 | 32.656 | 57.579 | 1.00 | 26.61 | C |
| ATOM | 2070 | CB | LEU | C | 71 | −16.540 | 32.879 | 56.101 | 1.00 | 28.42 | C |
| ATOM | 2071 | CG | LEU | C | 71 | −15.586 | 34.001 | 55.675 | 1.00 | 23.99 | C |
| ATOM | 2072 | CD1 | LEU | C | 71 | −15.458 | 34.033 | 54.173 | 1.00 | 18.21 | C |
| ATOM | 2073 | CD2 | LEU | C | 71 | −16.104 | 35.317 | 56.158 | 1.00 | 25.86 | C |
| ATOM | 2074 | C | LEU | C | 71 | −17.737 | 31.378 | 57.727 | 1.00 | 29.32 | C |
| ATOM | 2075 | O | LEU | C | 71 | −17.193 | 30.318 | 58.054 | 1.00 | 28.01 | C |
| ATOM | 2076 | N | CYS | C | 72 | −19.048 | 31.475 | 57.532 | 1.00 | 27.51 | C |
| ATOM | 2077 | CA | CYS | C | 72 | −19.908 | 30.303 | 57.604 | 1.00 | 27.51 | C |
| ATOM | 2078 | CB | CYS | C | 72 | −20.487 | 30.092 | 59.011 | 1.00 | 22.79 | C |
| ATOM | 2079 | SG | CYS | C | 72 | −21.731 | 31.271 | 59.537 | 1.00 | 27.60 | C |
| ATOM | 2080 | C | CYS | C | 72 | −21.027 | 30.463 | 56.595 | 1.00 | 29.16 | C |
| ATOM | 2081 | O | CYS | C | 72 | −21.201 | 31.535 | 56.003 | 1.00 | 30.53 | C |
| ATOM | 2082 | N | SER | C | 73 | −21.773 | 29.386 | 56.393 | 1.00 | 31.33 | C |
| ATOM | 2083 | CA | SER | C | 73 | −22.865 | 29.379 | 55.440 | 1.00 | 34.25 | C |
| ATOM | 2084 | CB | SER | C | 73 | −23.375 | 27.948 | 55.229 | 1.00 | 34.47 | C |
| ATOM | 2085 | OG | SER | C | 73 | −22.388 | 27.148 | 54.603 | 1.00 | 37.57 | C |
| ATOM | 2086 | C | SER | C | 73 | −24.009 | 30.251 | 55.893 | 1.00 | 33.98 | C |
| ATOM | 2087 | O | SER | C | 73 | −24.273 | 30.354 | 57.085 | 1.00 | 35.04 | C |
| ATOM | 2088 | N | VAL | C | 74 | −24.670 | 30.889 | 54.932 | 1.00 | 35.67 | C |
| ATOM | 2089 | CA | VAL | C | 74 | −25.828 | 31.719 | 55.222 | 1.00 | 40.21 | C |
| ATOM | 2090 | CB | VAL | C | 74 | −26.294 | 32.545 | 53.997 | 1.00 | 41.17 | C |
| ATOM | 2091 | CG1 | VAL | C | 74 | −27.470 | 33.419 | 54.385 | 1.00 | 41.13 | C |
| ATOM | 2092 | CG2 | VAL | C | 74 | −25.162 | 33.400 | 53.486 | 1.00 | 47.12 | C |
| ATOM | 2093 | C | VAL | C | 74 | −26.922 | 30.715 | 55.533 | 1.00 | 38.40 | C |
| ATOM | 2094 | O | VAL | C | 74 | −27.102 | 29.745 | 54.795 | 1.00 | 38.40 | C |
| ATOM | 2095 | N | LEU | C | 75 | −27.639 | 30.940 | 56.626 | 1.00 | 36.95 | C |
| ATOM | 2096 | CA | LEU | C | 75 | −28.706 | 30.039 | 57.023 | 1.00 | 36.14 | C |
| ATOM | 2097 | CB | LEU | C | 75 | −28.639 | 29.751 | 58.530 | 1.00 | 30.71 | C |
| ATOM | 2098 | CG | LEU | C | 75 | −27.570 | 28.755 | 58.971 | 1.00 | 31.12 | C |
| ATOM | 2099 | CD1 | LEU | C | 75 | −27.556 | 28.603 | 60.510 | 1.00 | 24.51 | C |
| ATOM | 2100 | CD2 | LEU | C | 75 | −27.848 | 27.425 | 58.270 | 1.00 | 22.61 | C |
| ATOM | 2101 | C | LEU | C | 75 | −30.028 | 30.684 | 56.709 | 1.00 | 36.90 | C |
| ATOM | 2102 | O | LEU | C | 75 | −30.091 | 31.891 | 56.501 | 1.00 | 39.23 | C |
| ATOM | 2103 | N | PRO | C | 76 | −31.100 | 29.881 | 56.638 | 1.00 | 38.41 | C |
| ATOM | 2104 | CD | PRO | C | 76 | −31.107 | 28.406 | 56.611 | 1.00 | 37.97 | C |
| ATOM | 2105 | CA | PRO | C | 76 | −32.434 | 30.422 | 56.355 | 1.00 | 36.75 | C |
| ATOM | 2106 | CB | PRO | C | 76 | −33.304 | 29.174 | 56.246 | 1.00 | 38.25 | C |
| ATOM | 2107 | CG | PRO | C | 76 | −32.341 | 28.115 | 55.794 | 1.00 | 43.03 | C |
| ATOM | 2108 | C | PRO | C | 76 | −32.800 | 31.247 | 57.584 | 1.00 | 38.15 | C |
| ATOM | 2109 | O | PRO | C | 76 | −32.376 | 30.925 | 58.692 | 1.00 | 40.29 | C |
| ATOM | 2110 | N | THR | C | 77 | −33.575 | 32.305 | 57.400 | 1.00 | 39.34 | C |
| ATOM | 2111 | CA | THR | C | 77 | −33.977 | 33.156 | 58.514 | 1.00 | 38.66 | C |
| ATOM | 2112 | CB | THR | C | 77 | −34.687 | 34.410 | 57.983 | 1.00 | 40.84 | C |
| ATOM | 2113 | OG1 | THR | C | 77 | −33.697 | 35.330 | 57.512 | 1.00 | 44.54 | C |
| ATOM | 2114 | CG2 | THR | C | 77 | −35.543 | 35.067 | 59.061 | 1.00 | 40.42 | C |
| ATOM | 2115 | C | THR | C | 77 | −34.900 | 32.421 | 59.478 | 1.00 | 40.63 | C |
| ATOM | 2116 | O | THR | C | 77 | −34.829 | 32.602 | 60.700 | 1.00 | 38.63 | C |
| ATOM | 2117 | N | HIS | C | 78 | −35.766 | 31.595 | 58.903 | 1.00 | 39.96 | C |
| ATOM | 2118 | CA | HIS | C | 78 | −36.742 | 30.820 | 59.651 | 1.00 | 40.41 | C |
| ATOM | 2119 | CB | HIS | C | 78 | −38.125 | 31.462 | 59.512 | 1.00 | 38.67 | C |
| ATOM | 2120 | CG | HIS | C | 78 | −39.181 | 30.834 | 60.365 | 1.00 | 40.89 | C |
| ATOM | 2121 | CD2 | HIS | C | 78 | −39.124 | 29.820 | 61.261 | 1.00 | 41.72 | C |
| ATOM | 2122 | ND1 | HIS | C | 78 | −40.489 | 31.275 | 60.373 | 1.00 | 38.88 | C |
| ATOM | 2123 | CE1 | HIS | C | 78 | −41.187 | 30.564 | 61.237 | 1.00 | 38.79 | C |
| ATOM | 2124 | NE2 | HIS | C | 78 | −40.383 | 29.674 | 61.791 | 1.00 | 43.14 | C |
| ATOM | 2125 | C | HIS | C | 78 | −36.742 | 29.425 | 59.041 | 1.00 | 40.68 | C |
| ATOM | 2126 | O | HIS | C | 78 | −36.702 | 29.272 | 57.819 | 1.00 | 43.76 | C |
| ATOM | 2127 | N | TRP | C | 79 | −36.771 | 28.407 | 59.888 | 1.00 | 36.36 | C |
| ATOM | 2128 | CA | TRP | C | 79 | −36.757 | 27.046 | 59.396 | 1.00 | 32.70 | C |
| ATOM | 2129 | CB | TRP | C | 79 | −35.327 | 26.562 | 59.251 | 1.00 | 29.75 | C |
| ATOM | 2130 | CG | TRP | C | 79 | −35.209 | 25.465 | 58.282 | 1.00 | 30.22 | C |
| ATOM | 2131 | CD2 | TRP | C | 79 | −35.464 | 25.545 | 56.882 | 1.00 | 27.92 | C |
| ATOM | 2132 | CE2 | TRP | C | 79 | −35.185 | 24.277 | 56.336 | 1.00 | 29.39 | C |
| ATOM | 2133 | CE3 | TRP | C | 79 | −35.901 | 26.566 | 56.035 | 1.00 | 30.13 | C |
| ATOM | 2134 | CD1 | TRP | C | 79 | −34.805 | 24.193 | 58.530 | 1.00 | 31.38 | C |
| ATOM | 2135 | NE1 | TRP | C | 79 | −34.785 | 23.468 | 57.365 | 1.00 | 31.51 | C |
| ATOM | 2136 | CZ2 | TRP | C | 79 | −35.326 | 24.000 | 54.973 | 1.00 | 29.40 | C |
| ATOM | 2137 | CZ3 | TRP | C | 79 | −36.042 | 26.291 | 54.681 | 1.00 | 31.68 | C |
| ATOM | 2138 | CH2 | TRP | C | 79 | −35.753 | 25.016 | 54.164 | 1.00 | 27.90 | C |
| ATOM | 2139 | C | TRP | C | 79 | −37.523 | 26.102 | 60.305 | 1.00 | 32.57 | C |
| ATOM | 2140 | O | TRP | C | 79 | −37.702 | 26.365 | 61.494 | 1.00 | 31.61 | C |
| ATOM | 2141 | N | ARG | C | 80 | −37.973 | 24.992 | 59.737 | 1.00 | 34.09 | C |
| ATOM | 2142 | CA | ARG | C | 80 | −38.731 | 24.026 | 60.506 | 1.00 | 35.79 | C |
| ATOM | 2143 | CB | ARG | C | 80 | −39.606 | 23.167 | 59.581 | 1.00 | 35.49 | C |
| ATOM | 2144 | CG | ARG | C | 80 | −40.293 | 22.012 | 60.291 | 1.00 | 38.40 | C |
| ATOM | 2145 | CD | ARG | C | 80 | −41.158 | 21.213 | 59.345 | 1.00 | 40.84 | C |
| ATOM | 2146 | NE | ARG | C | 80 | −42.406 | 21.904 | 59.047 | 1.00 | 43.22 | C |
| ATOM | 2147 | CZ | ARG | C | 80 | −43.338 | 21.441 | 58.222 | 1.00 | 43.11 | C |
| ATOM | 2148 | NH1 | ARG | C | 80 | −43.161 | 20.278 | 57.603 | 1.00 | 42.49 | C |
| ATOM | 2149 | NH2 | ARG | C | 80 | −44.456 | 22.132 | 58.033 | 1.00 | 37.61 | C |
| ATOM | 2150 | C | ARG | C | 80 | −37.840 | 23.131 | 61.358 | 1.00 | 34.71 | C |
| ATOM | 2151 | O | ARG | C | 80 | −36.778 | 22.674 | 60.931 | 1.00 | 30.77 | C |
| ATOM | 2152 | N | CYS | C | 81 | −38.303 | 22.889 | 62.573 | 1.00 | 34.67 | C |
| ATOM | 2153 | CA | CYS | C | 81 | −37.607 | 22.044 | 63.519 | 1.00 | 34.88 | C |
| ATOM | 2154 | CB | CYS | C | 81 | −38.486 | 21.927 | 64.768 | 1.00 | 31.37 | C |
| ATOM | 2155 | SG | CYS | C | 81 | −38.399 | 20.409 | 65.673 | 1.00 | 50.90 | C |
| ATOM | 2156 | C | CYS | C | 81 | −37.291 | 20.680 | 62.882 | 1.00 | 33.61 | C |
| ATOM | 2157 | O | CYS | C | 81 | −38.156 | 20.027 | 62.304 | 1.00 | 34.69 | C |
| ATOM | 2158 | N | ASN | C | 82 | −36.025 | 20.287 | 62.960 | 1.00 | 34.12 | C |
| ATOM | 2159 | CA | ASN | C | 82 | −35.523 | 19.023 | 62.424 | 1.00 | 30.63 | C |
| ATOM | 2160 | CB | ASN | C | 82 | −36.160 | 17.842 | 63.162 | 1.00 | 29.59 | C |
| ATOM | 2161 | CG | ASN | C | 82 | −35.436 | 16.534 | 62.885 | 1.00 | 31.48 | C |
| ATOM | 2162 | OD1 | ASN | C | 82 | −34.204 | 16.498 | 62.805 | 1.00 | 33.61 | C |
| ATOM | 2163 | ND2 | ASN | C | 82 | −36.192 | 15.453 | 62.744 | 1.00 | 33.04 | C |
| ATOM | 2164 | C | ASN | C | 82 | −35.627 | 18.796 | 60.912 | 1.00 | 31.43 | C |
| ATOM | 2165 | O | ASN | C | 82 | −35.462 | 17.677 | 60.439 | 1.00 | 31.86 | C |
| ATOM | 2166 | N | LYS | C | 83 | −35.879 | 19.848 | 60.147 | 1.00 | 32.65 | C |
| ATOM | 2167 | CA | LYS | C | 83 | −35.971 | 19.708 | 58.699 | 1.00 | 33.64 | C |
| ATOM | 2168 | CB | LYS | C | 83 | −36.978 | 20.726 | 58.145 | 1.00 | 35.90 | C |
| ATOM | 2169 | CG | LYS | C | 83 | −37.206 | 20.703 | 56.632 | 1.00 | 35.53 | C |
| ATOM | 2170 | CD | LYS | C | 83 | −38.315 | 21.687 | 56.287 | 1.00 | 43.16 | C |
| ATOM | 2171 | CE | LYS | C | 83 | −38.331 | 22.105 | 54.819 | 1.00 | 41.81 | C |
| ATOM | 2172 | NZ | LYS | C | 83 | −38.858 | 21.058 | 53.917 | 1.00 | 45.98 | C |
| ATOM | 2173 | C | LYS | C | 83 | −34.578 | 19.936 | 58.109 | 1.00 | 36.45 | C |
| ATOM | 2174 | O | LYS | C | 83 | −33.814 | 20.764 | 58.612 | 1.00 | 39.62 | C |
| ATOM | 2175 | N | THR | C | 84 | −34.236 | 19.194 | 57.061 | 1.00 | 34.66 | C |
| ATOM | 2176 | CA | THR | C | 84 | −32.932 | 19.350 | 56.432 | 1.00 | 35.55 | C |
| ATOM | 2177 | CB | THR | C | 84 | −32.745 | 18.360 | 55.254 | 1.00 | 34.77 | C |
| ATOM | 2178 | OG1 | THR | C | 84 | −32.632 | 17.027 | 55.765 | 1.00 | 37.42 | C |
| ATOM | 2179 | CG2 | THR | C | 84 | −31.483 | 18.691 | 54.465 | 1.00 | 33.12 | C |
| ATOM | 2180 | C | THR | C | 84 | −32.779 | 20.775 | 55.912 | 1.00 | 38.12 | C |
| ATOM | 2181 | O | THR | C | 84 | −33.738 | 21.373 | 55.429 | 1.00 | 39.49 | C |
| ATOM | 2182 | N | LEU | C | 85 | −31.577 | 21.326 | 56.023 | 1.00 | 39.88 | C |
| ATOM | 2183 | CA | LEU | C | 85 | −31.336 | 22.683 | 55.541 | 1.00 | 38.74 | C |
| ATOM | 2184 | CB | LEU | C | 85 | −30.021 | 23.223 | 56.108 | 1.00 | 37.40 | C |
| ATOM | 2185 | CG | LEU | C | 85 | −30.060 | 23.450 | 57.621 | 1.00 | 38.73 | C |
| ATOM | 2186 | CD1 | LEU | C | 85 | −28.648 | 23.680 | 58.152 | 1.00 | 34.16 | C |
| ATOM | 2187 | CD2 | LEU | C | 85 | −30.987 | 24.620 | 57.929 | 1.00 | 32.68 | C |
| ATOM | 2188 | C | LEU | C | 85 | −31.301 | 22.682 | 54.021 | 1.00 | 36.41 | C |
| ATOM | 2189 | O | LEU | C | 85 | −30.950 | 21.681 | 53.402 | 1.00 | 34.24 | C |
| ATOM | 2190 | N | PRO | C | 86 | −31.690 | 23.801 | 53.401 | 1.00 | 35.90 | C |
| ATOM | 2191 | CD | PRO | C | 86 | −32.248 | 25.001 | 54.046 | 1.00 | 36.58 | C |
| ATOM | 2192 | CA | PRO | C | 86 | −31.707 | 23.935 | 51.941 | 1.00 | 38.25 | C |
| ATOM | 2193 | CB | PRO | C | 86 | −32.493 | 25.231 | 51.729 | 1.00 | 37.13 | C |
| ATOM | 2194 | CG | PRO | C | 86 | −32.168 | 26.024 | 52.948 | 1.00 | 37.77 | C |
| ATOM | 2195 | C | PRO | C | 86 | −30.309 | 23.966 | 51.304 | 1.00 | 40.07 | C |
| ATOM | 2196 | O | PRO | C | 86 | −30.170 | 23.843 | 50.087 | 1.00 | 41.00 | C |
| ATOM | 2197 | N | ILE | C | 87 | −29.287 | 24.129 | 52.143 | 1.00 | 39.29 | C |
| ATOM | 2198 | CA | ILE | C | 87 | −27.887 | 24.166 | 51.717 | 1.00 | 38.33 | C |
| ATOM | 2199 | CB | ILE | C | 87 | −27.364 | 25.642 | 51.593 | 1.00 | 41.38 | C |
| ATOM | 2200 | CG2 | ILE | C | 87 | −28.195 | 26.401 | 50.573 | 1.00 | 41.48 | C |
| ATOM | 2201 | CG1 | ILE | C | 87 | −27.466 | 26.387 | 52.938 | 1.00 | 44.39 | C |
| ATOM | 2202 | CD1 | ILE | C | 87 | −26.379 | 26.052 | 53.960 | 1.00 | 43.12 | C |
| ATOM | 2203 | C | ILE | C | 87 | −27.096 | 23.428 | 52.793 | 1.00 | 35.39 | C |
| ATOM | 2204 | O | ILE | C | 87 | −27.558 | 23.319 | 53.920 | 1.00 | 36.57 | C |
| ATOM | 2205 | N | ALA | C | 88 | −25.917 | 22.916 | 52.458 | 1.00 | 32.88 | C |
| ATOM | 2206 | CA | ALA | C | 88 | −25.095 | 22.206 | 53.440 | 1.00 | 31.82 | C |
| ATOM | 2207 | CB | ALA | C | 88 | −24.186 | 21.205 | 52.741 | 1.00 | 27.94 | C |
| ATOM | 2208 | C | ALA | C | 88 | −24.253 | 23.206 | 54.234 | 1.00 | 32.23 | C |
| ATOM | 2209 | O | ALA | C | 88 | −23.427 | 23.925 | 53.662 | 1.00 | 33.93 | C |
| ATOM | 2210 | N | PHE | C | 89 | −24.445 | 23.243 | 55.547 | 1.00 | 29.93 | C |
| ATOM | 2211 | CA | PHE | C | 89 | −23.695 | 24.172 | 56.383 | 1.00 | 33.13 | C |
| ATOM | 2212 | CB | PHE | C | 89 | −24.115 | 24.061 | 57.846 | 1.00 | 28.93 | C |
| ATOM | 2213 | CG | PHE | C | 89 | −23.634 | 25.203 | 58.680 | 1.00 | 29.06 | C |
| ATOM | 2214 | CD1 | PHE | C | 89 | −24.099 | 26.494 | 58.435 | 1.00 | 29.92 | C |
| ATOM | 2215 | CD2 | PHE | C | 89 | −22.700 | 25.005 | 59.689 | 1.00 | 25.08 | C |
| ATOM | 2216 | CE1 | PHE | C | 89 | −23.642 | 27.577 | 59.185 | 1.00 | 26.73 | C |
| ATOM | 2217 | CE2 | PHE | C | 89 | −22.234 | 26.078 | 60.445 | 1.00 | 27.16 | C |
| ATOM | 2218 | CZ | PHE | C | 89 | −22.708 | 27.371 | 60.190 | 1.00 | 28.28 | C |
| ATOM | 2219 | C | PHE | C | 89 | −22.181 | 23.997 | 56.313 | 1.00 | 34.47 | C |
| ATOM | 2220 | O | PHE | C | 89 | −21.660 | 22.894 | 56.512 | 1.00 | 38.01 | C |
| ATOM | 2221 | N | LYS | C | 90 | −21.478 | 25.095 | 56.049 | 1.00 | 32.15 | C |
| ATOM | 2222 | CA | LYS | C | 90 | −20.020 | 25.069 | 55.972 | 1.00 | 34.17 | C |
| ATOM | 2223 | CB | LYS | C | 90 | −19.528 | 25.317 | 54.541 | 1.00 | 34.96 | C |
| ATOM | 2224 | CG | LYS | C | 90 | −19.916 | 24.279 | 53.513 | 1.00 | 41.90 | C |
| ATOM | 2225 | CD | LYS | C | 90 | −19.718 | 24.850 | 52.109 | 1.00 | 46.04 | C |
| ATOM | 2226 | CE | LYS | C | 90 | −20.420 | 24.011 | 51.052 | 1.00 | 48.91 | C |
| ATOM | 2227 | NZ | LYS | C | 90 | −19.753 | 22.692 | 50.872 | 1.00 | 51.57 | C |
| ATOM | 2228 | C | LYS | C | 90 | −19.429 | 26.163 | 56.853 | 1.00 | 34.46 | C |
| ATOM | 2229 | O | LYS | C | 90 | −20.013 | 27.243 | 56.997 | 1.00 | 29.80 | C |
| ATOM | 2230 | N | VAL | C | 91 | −18.279 | 25.865 | 57.454 | 1.00 | 33.33 | C |
| ATOM | 2231 | CA | VAL | C | 91 | −17.555 | 26.834 | 58.261 | 1.00 | 29.64 | C |
| ATOM | 2232 | CB | VAL | C | 91 | −17.228 | 26.306 | 59.660 | 1.00 | 29.02 | C |
| ATOM | 2233 | CG1 | VAL | C | 91 | −16.301 | 27.286 | 60.368 | 1.00 | 26.17 | C |
| ATOM | 2234 | CG2 | VAL | C | 91 | −18.512 | 26.133 | 60.472 | 1.00 | 28.25 | C |
| ATOM | 2235 | C | VAL | C | 91 | −16.260 | 26.995 | 57.478 | 1.00 | 29.47 | C |
| ATOM | 2236 | O | VAL | C | 91 | −15.572 | 26.015 | 57.220 | 1.00 | 31.84 | C |
| ATOM | 2237 | N | VAL | C | 92 | −15.948 | 28.215 | 57.056 | 1.00 | 27.86 | C |
| ATOM | 2238 | CA | VAL | C | 92 | −14.723 | 28.453 | 56.301 | 1.00 | 26.10 | C |
| ATOM | 2239 | CB | VAL | C | 92 | −14.986 | 29.288 | 55.023 | 1.00 | 26.95 | C |
| ATOM | 2240 | CG1 | VAL | C | 92 | −13.667 | 29.522 | 54.281 | 1.00 | 20.20 | C |
| ATOM | 2241 | CG2 | VAL | C | 92 | −15.997 | 28.569 | 54.117 | 1.00 | 21.64 | C |
| ATOM | 2242 | C | VAL | C | 92 | −13.685 | 29.185 | 57.140 | 1.00 | 26.50 | C |
| ATOM | 2243 | O | VAL | C | 92 | −13.941 | 30.275 | 57.653 | 1.00 | 24.74 | C |
| ATOM | 2244 | N | ALA | C | 93 | −12.514 | 28.573 | 57.285 | 1.00 | 28.12 | C |
| ATOM | 2245 | CA | ALA | C | 93 | −11.436 | 29.186 | 58.048 | 1.00 | 29.15 | C |
| ATOM | 2246 | CB | ALA | C | 93 | −10.583 | 28.132 | 58.700 | 1.00 | 18.92 | C |
| ATOM | 2247 | C | ALA | C | 93 | −10.597 | 30.024 | 57.108 | 1.00 | 30.42 | C |
| ATOM | 2248 | O | ALA | C | 93 | −10.171 | 29.543 | 56.054 | 1.00 | 33.81 | C |
| ATOM | 2249 | N | LEU | C | 94 | −10.376 | 31.284 | 57.474 | 1.00 | 32.11 | C |
| ATOM | 2250 | CA | LEU | C | 94 | −9.563 | 32.172 | 56.646 | 1.00 | 30.55 | C |
| ATOM | 2251 | CB | LEU | C | 94 | −9.791 | 33.639 | 57.028 | 1.00 | 25.20 | C |
| ATOM | 2252 | CG | LEU | C | 94 | −11.243 | 34.140 | 56.914 | 1.00 | 27.89 | C |
| ATOM | 2253 | CD1 | LEU | C | 94 | −11.282 | 35.628 | 57.206 | 1.00 | 26.21 | C |
| ATOM | 2254 | CD2 | LEU | C | 94 | −11.809 | 33.872 | 55.522 | 1.00 | 20.22 | C |
| ATOM | 2255 | C | LEU | C | 94 | −8.112 | 31.778 | 56.877 | 1.00 | 30.67 | C |
| ATOM | 2256 | O | LEU | C | 94 | −7.494 | 31.130 | 56.025 | 1.00 | 31.46 | C |
| ATOM | 2257 | N | GLY | C | 95 | −7.577 | 32.143 | 58.034 | 1.00 | 24.96 | C |
| ATOM | 2258 | CA | GLY | C | 95 | −6.205 | 31.796 | 58.335 | 1.00 | 25.58 | C |
| ATOM | 2259 | C | GLY | C | 95 | −5.988 | 30.298 | 58.483 | 1.00 | 29.63 | C |
| ATOM | 2260 | O | GLY | C | 95 | −6.899 | 29.547 | 58.871 | 1.00 | 31.58 | C |
| ATOM | 2261 | N | ASP | C | 96 | −4.771 | 29.860 | 58.186 | 1.00 | 27.73 | C |
| ATOM | 2262 | CA | ASP | C | 96 | −4.420 | 28.445 | 58.265 | 1.00 | 31.37 | C |
| ATOM | 2263 | CB | ASP | C | 96 | −2.890 | 28.263 | 58.318 | 1.00 | 30.07 | C |
| ATOM | 2264 | CG | ASP | C | 96 | −2.184 | 28.646 | 57.016 | 1.00 | 39.54 | C |
| ATOM | 2265 | OD1 | ASP | C | 96 | −2.730 | 29.434 | 56.211 | 1.00 | 42.05 | C |
| ATOM | 2266 | OD2 | ASP | C | 96 | −1.047 | 28.163 | 56.809 | 1.00 | 44.03 | C |
| ATOM | 2267 | C | ASP | C | 96 | −5.017 | 27.727 | 59.483 | 1.00 | 32.25 | C |
| ATOM | 2268 | O | ASP | C | 96 | −4.991 | 28.253 | 60.604 | 1.00 | 32.60 | C |
| ATOM | 2269 | N | VAL | C | 97 | −5.558 | 26.534 | 59.237 | 1.00 | 27.90 | C |
| ATOM | 2270 | CA | VAL | C | 97 | −6.101 | 25.659 | 60.276 | 1.00 | 31.30 | C |
| ATOM | 2271 | CB | VAL | C | 97 | −7.605 | 25.867 | 60.523 | 1.00 | 32.25 | C |
| ATOM | 2272 | CG1 | VAL | C | 97 | −8.138 | 24.751 | 61.420 | 1.00 | 31.35 | C |
| ATOM | 2273 | CG2 | VAL | C | 97 | −7.832 | 27.207 | 61.189 | 1.00 | 32.29 | C |
| ATOM | 2274 | C | VAL | C | 97 | −5.864 | 24.257 | 59.733 | 1.00 | 29.45 | C |
| ATOM | 2275 | O | VAL | C | 97 | −6.371 | 23.898 | 58.684 | 1.00 | 31.48 | C |
| ATOM | 2276 | N | PRO | C | 98 | −5.083 | 23.445 | 60.445 | 1.00 | 30.86 | C |
| ATOM | 2277 | CD | PRO | C | 98 | −4.395 | 23.734 | 61.717 | 1.00 | 25.22 | C |
| ATOM | 2278 | CA | PRO | C | 98 | −4.799 | 22.084 | 59.970 | 1.00 | 29.68 | C |
| ATOM | 2279 | CB | PRO | C | 98 | −3.812 | 21.549 | 61.016 | 1.00 | 28.70 | C |
| ATOM | 2280 | CG | PRO | C | 98 | −4.105 | 22.361 | 62.241 | 1.00 | 28.52 | C |
| ATOM | 2281 | C | PRO | C | 98 | −5.968 | 21.138 | 59.732 | 1.00 | 31.46 | C |
| ATOM | 2282 | O | PRO | C | 98 | −6.914 | 21.078 | 60.526 | 1.00 | 32.86 | C |
| ATOM | 2283 | N | ASP | C | 99 | −5.889 | 20.399 | 58.625 | 1.00 | 31.31 | C |
| ATOM | 2284 | CA | ASP | C | 99 | −6.910 | 19.417 | 58.276 | 1.00 | 34.49 | C |
| ATOM | 2285 | CB | ASP | C | 99 | −6.461 | 18.522 | 57.116 | 1.00 | 38.17 | C |
| ATOM | 2286 | CG | ASP | C | 99 | −6.408 | 19.242 | 55.786 | 1.00 | 44.07 | C |
| ATOM | 2287 | OD1 | ASP | C | 99 | −7.065 | 20.296 | 55.628 | 1.00 | 47.55 | C |
| ATOM | 2288 | OD2 | ASP | C | 99 | −5.711 | 18.724 | 54.883 | 1.00 | 45.48 | C |
| ATOM | 2289 | C | ASP | C | 99 | −7.154 | 18.504 | 59.473 | 1.00 | 35.28 | C |
| ATOM | 2290 | O | ASP | C | 99 | −6.241 | 18.231 | 60.249 | 1.00 | 36.07 | C |
| ATOM | 2291 | N | GLY | C | 100 | −8.383 | 18.018 | 59.611 | 1.00 | 33.38 | C |
| ATOM | 2292 | CA | GLY | C | 100 | −8.683 | 17.128 | 60.709 | 1.00 | 29.75 | C |
| ATOM | 2293 | C | GLY | C | 100 | −9.160 | 17.835 | 61.952 | 1.00 | 30.53 | C |
| ATOM | 2294 | O | GLY | C | 100 | −9.792 | 17.202 | 62.801 | 1.00 | 32.37 | C |
| ATOM | 2295 | N | THR | C | 101 | −8.870 | 19.134 | 62.064 | 1.00 | 27.85 | C |
| ATOM | 2296 | CA | THR | C | 101 | −9.276 | 19.926 | 63.223 | 1.00 | 25.69 | C |
| ATOM | 2297 | CB | THR | C | 101 | −8.883 | 21.406 | 63.062 | 1.00 | 28.49 | C |
| ATOM | 2298 | OG1 | THR | C | 101 | −7.462 | 21.517 | 62.897 | 1.00 | 30.37 | C |
| ATOM | 2299 | CG2 | THR | C | 101 | −9.324 | 22.205 | 64.289 | 1.00 | 20.01 | C |
| ATOM | 2300 | C | THR | C | 101 | −10.786 | 19.863 | 63.370 | 1.00 | 29.15 | C |
| ATOM | 2301 | O | THR | C | 101 | −11.518 | 20.154 | 62.422 | 1.00 | 29.95 | C |
| ATOM | 2302 | N | LEU | C | 102 | −11.256 | 19.493 | 64.556 | 1.00 | 30.63 | C |
| ATOM | 2303 | CA | LEU | C | 102 | −12.690 | 19.387 | 64.799 | 1.00 | 30.79 | C |
| ATOM | 2304 | CB | LEU | C | 102 | −12.953 | 18.547 | 66.044 | 1.00 | 28.51 | C |
| ATOM | 2305 | CG | LEU | C | 102 | −12.380 | 17.128 | 65.910 | 1.00 | 36.23 | C |
| ATOM | 2306 | CD1 | LEU | C | 102 | −12.381 | 16.440 | 67.266 | 1.00 | 37.67 | C |
| ATOM | 2307 | CD2 | LEU | C | 102 | −13.171 | 16.330 | 64.895 | 1.00 | 33.96 | C |
| ATOM | 2308 | C | LEU | C | 102 | −13.412 | 20.725 | 64.924 | 1.00 | 32.89 | C |
| ATOM | 2309 | O | LEU | C | 102 | −12.928 | 21.669 | 65.555 | 1.00 | 34.47 | C |
| ATOM | 2310 | N | VAL | C | 103 | −14.576 | 20.801 | 64.292 | 1.00 | 29.35 | C |
| ATOM | 2311 | CA | VAL | C | 103 | −15.394 | 21.990 | 64.355 | 1.00 | 26.95 | C |
| ATOM | 2312 | CB | VAL | C | 103 | −15.513 | 22.654 | 62.988 | 1.00 | 24.98 | C |
| ATOM | 2313 | CG1 | VAL | C | 103 | −16.294 | 23.937 | 63.118 | 1.00 | 21.66 | C |
| ATOM | 2314 | CG2 | VAL | C | 103 | −14.135 | 22.918 | 62.421 | 1.00 | 28.10 | C |
| ATOM | 2315 | C | VAL | C | 103 | −16.783 | 21.566 | 64.822 | 1.00 | 26.32 | C |
| ATOM | 2316 | O | VAL | C | 103 | −17.374 | 20.635 | 64.274 | 1.00 | 28.87 | C |
| ATOM | 2317 | N | THR | C | 104 | −17.293 | 22.227 | 65.850 | 1.00 | 24.59 | C |
| ATOM | 2318 | CA | THR | C | 104 | −18.618 | 21.906 | 66.336 | 1.00 | 27.26 | C |
| ATOM | 2319 | CB | THR | C | 104 | −18.619 | 21.396 | 67.799 | 1.00 | 28.77 | C |
| ATOM | 2320 | OG1 | THR | C | 104 | −18.353 | 22.482 | 68.694 | 1.00 | 32.01 | C |
| ATOM | 2321 | CG2 | THR | C | 104 | −17.566 | 20.316 | 67.986 | 1.00 | 31.71 | C |
| ATOM | 2322 | C | THR | C | 104 | −19.452 | 23.161 | 66.259 | 1.00 | 28.14 | C |
| ATOM | 2323 | O | THR | C | 104 | −18.922 | 24.268 | 66.141 | 1.00 | 30.24 | C |
| ATOM | 2324 | N | VAL | C | 105 | −20.762 | 22.968 | 66.307 | 1.00 | 29.13 | C |
| ATOM | 2325 | CA | VAL | C | 105 | −21.724 | 24.055 | 66.255 | 1.00 | 29.87 | C |
| ATOM | 2326 | CB | VAL | C | 105 | −22.565 | 24.004 | 64.963 | 1.00 | 28.84 | C |
| ATOM | 2327 | CG1 | VAL | C | 105 | −23.610 | 25.090 | 64.981 | 1.00 | 34.13 | C |
| ATOM | 2328 | CG2 | VAL | C | 105 | −21.681 | 24.153 | 63.770 | 1.00 | 32.31 | C |
| ATOM | 2329 | C | VAL | C | 105 | −22.686 | 23.879 | 67.424 | 1.00 | 31.67 | C |
| ATOM | 2330 | O | VAL | C | 105 | −22.985 | 22.752 | 67.834 | 1.00 | 33.85 | C |
| ATOM | 2331 | N | MET | C | 106 | −23.164 | 24.998 | 67.955 | 1.00 | 28.11 | C |
| ATOM | 2332 | CA | MET | C | 106 | −24.134 | 24.983 | 69.031 | 1.00 | 25.41 | C |
| ATOM | 2333 | CB | MET | C | 106 | −23.457 | 25.185 | 70.380 | 1.00 | 23.76 | C |
| ATOM | 2334 | CG | MET | C | 106 | −22.458 | 24.107 | 70.699 | 1.00 | 24.51 | C |
| ATOM | 2335 | SD | MET | C | 106 | −21.995 | 24.096 | 72.433 | 1.00 | 35.20 | C |
| ATOM | 2336 | CE | MET | C | 106 | −21.065 | 25.615 | 72.578 | 1.00 | 23.30 | C |
| ATOM | 2337 | C | MET | C | 106 | −25.092 | 26.120 | 68.739 | 1.00 | 26.71 | C |
| ATOM | 2338 | O | MET | C | 106 | −24.719 | 27.096 | 68.081 | 1.00 | 26.62 | C |
| ATOM | 2339 | N | ALA | C | 107 | −26.330 | 25.988 | 69.203 | 1.00 | 26.98 | C |
| ATOM | 2340 | CA | ALA | C | 107 | −27.332 | 27.021 | 68.979 | 1.00 | 28.83 | C |
| ATOM | 2341 | CB | ALA | C | 107 | −28.242 | 26.629 | 67.828 | 1.00 | 29.04 | C |
| ATOM | 2342 | C | ALA | C | 107 | −28.152 | 27.260 | 70.232 | 1.00 | 31.43 | C |
| ATOM | 2343 | O | ALA | C | 107 | −28.545 | 26.324 | 70.920 | 1.00 | 31.42 | C |
| ATOM | 2344 | N | GLY | C | 108 | −28.398 | 28.527 | 70.532 | 1.00 | 30.92 | C |
| ATOM | 2345 | CA | GLY | C | 108 | −29.185 | 28.840 | 71.698 | 1.00 | 30.07 | C |
| ATOM | 2346 | C | GLY | C | 108 | −29.409 | 30.323 | 71.837 | 1.00 | 34.12 | C |
| ATOM | 2347 | O | GLY | C | 108 | −28.868 | 31.114 | 71.073 | 1.00 | 34.25 | C |
| ATOM | 2348 | N | ASN | C | 109 | −30.242 | 30.688 | 72.802 | 1.00 | 37.52 | C |
| ATOM | 2349 | CA | ASN | C | 109 | −30.522 | 32.078 | 73.104 | 1.00 | 41.79 | C |
| ATOM | 2350 | CB | ASN | C | 109 | −31.748 | 32.592 | 72.330 | 1.00 | 43.32 | C |
| ATOM | 2351 | CG | ASN | C | 109 | −32.936 | 31.658 | 72.406 | 1.00 | 44.21 | C |
| ATOM | 2352 | OD1 | ASN | C | 109 | −33.216 | 31.056 | 73.445 | 1.00 | 47.27 | C |
| ATOM | 2353 | ND2 | ASN | C | 109 | −33.658 | 31.548 | 71.303 | 1.00 | 46.40 | C |
| ATOM | 2354 | C | ASN | C | 109 | −30.731 | 32.166 | 74.613 | 1.00 | 45.76 | C |
| ATOM | 2355 | O | ASN | C | 109 | −30.242 | 31.313 | 75.342 | 1.00 | 47.24 | C |
| ATOM | 2356 | N | ASP | C | 110 | −31.450 | 33.181 | 75.078 | 1.00 | 50.37 | C |
| ATOM | 2357 | CA | ASP | C | 110 | −31.690 | 33.373 | 76.508 | 1.00 | 55.06 | C |
| ATOM | 2358 | CB | ASP | C | 110 | −32.341 | 34.735 | 76.740 | 1.00 | 58.47 | C |
| ATOM | 2359 | CG | ASP | C | 110 | −33.603 | 34.909 | 75.934 | 1.00 | 62.99 | C |
| ATOM | 2360 | OD1 | ASP | C | 110 | −33.507 | 34.981 | 74.692 | 1.00 | 67.69 | C |
| ATOM | 2361 | OD2 | ASP | C | 110 | −34.695 | 34.958 | 76.538 | 1.00 | 69.79 | C |
| ATOM | 2362 | C | ASP | C | 110 | −32.545 | 32.282 | 77.154 | 1.00 | 55.76 | C |
| ATOM | 2363 | O | ASP | C | 110 | −32.342 | 31.938 | 78.315 | 1.00 | 54.12 | C |
| ATOM | 2364 | N | GLU | C | 111 | −33.511 | 31.754 | 76.410 | 1.00 | 58.49 | C |
| ATOM | 2365 | CA | GLU | C | 111 | −34.380 | 30.692 | 76.922 | 1.00 | 59.19 | C |
| ATOM | 2366 | CB | GLU | C | 111 | −35.708 | 30.665 | 76.172 | 1.00 | 61.48 | C |
| ATOM | 2367 | CG | GLU | C | 111 | −36.538 | 31.906 | 76.322 | 1.00 | 70.63 | C |
| ATOM | 2368 | CD | GLU | C | 111 | −37.827 | 31.814 | 75.545 | 1.00 | 71.90 | C |
| ATOM | 2369 | OE1 | GLU | C | 111 | −38.548 | 30.809 | 75.726 | 1.00 | 72.19 | C |
| ATOM | 2370 | OE2 | GLU | C | 111 | −38.119 | 32.745 | 74.764 | 1.00 | 73.76 | C |
| ATOM | 2371 | C | GLU | C | 111 | −33.693 | 29.358 | 76.703 | 1.00 | 56.37 | C |
| ATOM | 2372 | O | GLU | C | 111 | −33.134 | 28.756 | 77.627 | 1.00 | 54.81 | C |
| ATOM | 2373 | N | ASN | C | 112 | −33.757 | 28.899 | 75.460 | 1.00 | 48.89 | C |
| ATOM | 2374 | CA | ASN | C | 112 | −33.139 | 27.645 | 75.088 | 1.00 | 45.44 | C |
| ATOM | 2375 | CB | ASN | C | 112 | −33.735 | 27.171 | 73.765 | 1.00 | 40.94 | C |
| ATOM | 2376 | CG | ASN | C | 112 | −33.512 | 25.710 | 73.527 | 1.00 | 41.15 | C |
| ATOM | 2377 | OD1 | ASN | C | 112 | −32.724 | 25.072 | 74.225 | 1.00 | 43.65 | C |
| ATOM | 2378 | ND2 | ASN | C | 112 | −34.193 | 25.162 | 72.532 | 1.00 | 38.21 | C |
| ATOM | 2379 | C | ASN | C | 112 | −31.636 | 27.938 | 74.958 | 1.00 | 45.15 | C |
| ATOM | 2380 | O | ASN | C | 112 | −31.168 | 28.420 | 73.923 | 1.00 | 43.37 | C |
| ATOM | 2381 | N | TYR | C | 113 | −30.896 | 27.670 | 76.032 | 1.00 | 42.09 | C |
| ATOM | 2382 | CA | TYR | C | 113 | −29.458 | 27.918 | 76.082 | 1.00 | 41.24 | C |
| ATOM | 2383 | CB | TYR | C | 113 | −28.929 | 27.687 | 77.500 | 1.00 | 44.97 | C |
| ATOM | 2384 | CG | TYR | C | 113 | −29.482 | 28.644 | 78.514 | 1.00 | 48.59 | C |
| ATOM | 2385 | CD1 | TYR | C | 113 | −30.190 | 28.186 | 79.626 | 1.00 | 52.29 | C |
| ATOM | 2386 | CE1 | TYR | C | 113 | −30.722 | 29.080 | 80.569 | 1.00 | 54.47 | C |
| ATOM | 2387 | CD2 | TYR | C | 113 | −29.310 | 30.015 | 78.361 | 1.00 | 49.69 | C |
| ATOM | 2388 | CE2 | TYR | C | 113 | −29.826 | 30.915 | 79.289 | 1.00 | 55.35 | C |
| ATOM | 2389 | CZ | TYR | C | 113 | −30.534 | 30.446 | 80.391 | 1.00 | 55.07 | C |
| ATOM | 2390 | OH | TYR | C | 113 | −31.052 | 31.353 | 81.295 | 1.00 | 56.26 | C |
| ATOM | 2391 | C | TYR | C | 113 | −28.654 | 27.057 | 75.124 | 1.00 | 40.30 | C |
| ATOM | 2392 | O | TYR | C | 113 | −27.616 | 27.476 | 74.617 | 1.00 | 41.74 | C |
| ATOM | 2393 | N | SER | C | 114 | −29.130 | 25.846 | 74.882 | 1.00 | 39.35 | C |
| ATOM | 2394 | CA | SER | C | 114 | −28.424 | 24.941 | 74.005 | 1.00 | 37.57 | C |
| ATOM | 2395 | CB | SER | C | 114 | −27.414 | 24.147 | 74.829 | 1.00 | 37.77 | C |
| ATOM | 2396 | OG | SER | C | 114 | −26.465 | 23.508 | 74.005 | 1.00 | 42.42 | C |
| ATOM | 2397 | C | SER | C | 114 | −29.419 | 24.012 | 73.330 | 1.00 | 38.29 | C |
| ATOM | 2398 | O | SER | C | 114 | −29.746 | 22.958 | 73.862 | 1.00 | 42.79 | C |
| ATOM | 2399 | N | ALA | C | 115 | −29.903 | 24.409 | 72.158 | 1.00 | 36.65 | C |
| ATOM | 2400 | CA | ALA | C | 115 | −30.870 | 23.614 | 71.407 | 1.00 | 35.98 | C |
| ATOM | 2401 | CB | ALA | C | 115 | −31.348 | 24.404 | 70.197 | 1.00 | 37.14 | C |
| ATOM | 2402 | C | ALA | C | 115 | −30.305 | 22.272 | 70.949 | 1.00 | 37.13 | C |
| ATOM | 2403 | O | ALA | C | 115 | −29.103 | 22.139 | 70.727 | 1.00 | 38.16 | C |
| ATOM | 2404 | N | GLU | C | 116 | −31.170 | 21.270 | 70.810 | 1.00 | 36.87 | C |
| ATOM | 2405 | CA | GLU | C | 116 | −30.706 | 19.973 | 70.346 | 1.00 | 37.22 | C |
| ATOM | 2406 | CB | GLU | C | 116 | −31.748 | 18.876 | 70.591 | 1.00 | 34.85 | C |
| ATOM | 2407 | CG | GLU | C | 116 | −31.442 | 17.590 | 69.816 | 1.00 | 38.89 | C |
| ATOM | 2408 | CD | GLU | C | 116 | −32.401 | 16.437 | 70.106 | 1.00 | 43.12 | C |
| ATOM | 2409 | OE1 | GLU | C | 116 | −33.551 | 16.696 | 70.527 | 1.00 | 44.28 | C |
| ATOM | 2410 | OE2 | GLU | C | 116 | −31.999 | 15.267 | 69.892 | 1.00 | 44.72 | C |
| ATOM | 2411 | C | GLU | C | 116 | −30.406 | 20.085 | 68.855 | 1.00 | 38.26 | C |
| ATOM | 2412 | O | GLU | C | 116 | −31.188 | 20.648 | 68.090 | 1.00 | 36.97 | C |
| ATOM | 2413 | N | LEU | C | 117 | −29.262 | 19.551 | 68.451 | 1.00 | 37.89 | C |
| ATOM | 2414 | CA | LEU | C | 117 | −28.856 | 19.592 | 67.062 | 1.00 | 37.08 | C |
| ATOM | 2415 | CB | LEU | C | 117 | −27.644 | 20.512 | 66.908 | 1.00 | 37.77 | C |
| ATOM | 2416 | CG | LEU | C | 117 | −27.804 | 21.963 | 67.382 | 1.00 | 41.21 | C |
| ATOM | 2417 | CD1 | LEU | C | 117 | −26.472 | 22.698 | 67.233 | 1.00 | 42.22 | C |
| ATOM | 2418 | CD2 | LEU | C | 117 | −28.881 | 22.660 | 66.568 | 1.00 | 39.29 | C |
| ATOM | 2419 | C | LEU | C | 117 | −28.497 | 18.192 | 66.592 | 1.00 | 36.94 | C |
| ATOM | 2420 | O | LEU | C | 117 | −28.311 | 17.284 | 67.399 | 1.00 | 37.86 | C |
| ATOM | 2421 | N | ARG | C | 118 | −28.416 | 18.018 | 65.280 | 1.00 | 33.70 | C |
| ATOM | 2422 | CA | ARG | C | 118 | −28.043 | 16.739 | 64.710 | 1.00 | 32.20 | C |
| ATOM | 2423 | CB | ARG | C | 118 | −29.207 | 16.114 | 63.939 | 1.00 | 36.79 | C |
| ATOM | 2424 | CG | ARG | C | 118 | −30.433 | 15.833 | 64.767 | 1.00 | 42.31 | C |
| ATOM | 2425 | CD | ARG | C | 118 | −30.135 | 14.858 | 65.894 | 1.00 | 43.92 | C |
| ATOM | 2426 | NE | ARG | C | 118 | −29.670 | 13.563 | 65.403 | 1.00 | 43.61 | C |
| ATOM | 2427 | CZ | ARG | C | 118 | −29.430 | 12.523 | 66.196 | 1.00 | 43.50 | C |
| ATOM | 2428 | NH1 | ARG | C | 118 | −29.611 | 12.638 | 67.508 | 1.00 | 41.76 | C |
| ATOM | 2429 | NH2 | ARG | C | 118 | −29.015 | 11.375 | 65.683 | 1.00 | 40.45 | C |
| ATOM | 2430 | C | ARG | C | 118 | −26.901 | 16.986 | 63.744 | 1.00 | 31.49 | C |
| ATOM | 2431 | O | ARG | C | 118 | −27.000 | 17.857 | 62.872 | 1.00 | 30.15 | C |
| ATOM | 2432 | N | ASN | C | 119 | −25.828 | 16.214 | 63.900 | 1.00 | 30.74 | C |
| ATOM | 2433 | CA | ASN | C | 119 | −24.658 | 16.310 | 63.037 | 1.00 | 29.31 | C |
| ATOM | 2434 | CB | ASN | C | 119 | −25.060 | 16.043 | 61.589 | 1.00 | 29.76 | C |
| ATOM | 2435 | CG | ASN | C | 119 | −25.946 | 14.840 | 61.466 | 1.00 | 29.59 | C |
| ATOM | 2436 | OD1 | ASN | C | 119 | −25.668 | 13.800 | 62.049 | 1.00 | 31.99 | C |
| ATOM | 2437 | ND2 | ASN | C | 119 | −27.030 | 14.973 | 60.720 | 1.00 | 36.26 | C |
| ATOM | 2438 | C | ASN | C | 119 | −24.016 | 17.669 | 63.154 | 1.00 | 29.76 | C |
| ATOM | 2439 | O | ASN | C | 119 | −23.739 | 18.325 | 62.156 | 1.00 | 31.14 | C |
| ATOM | 2440 | N | ALA | C | 120 | −23.776 | 18.074 | 64.396 | 1.00 | 33.36 | C |
| ATOM | 2441 | CA | ALA | C | 120 | −23.186 | 19.360 | 64.700 | 1.00 | 33.38 | C |
| ATOM | 2442 | CB | ALA | C | 120 | −23.869 | 19.942 | 65.918 | 1.00 | 30.18 | C |
| ATOM | 2443 | C | ALA | C | 120 | −21.673 | 19.281 | 64.920 | 1.00 | 34.62 | C |
| ATOM | 2444 | O | ALA | C | 120 | −21.116 | 20.020 | 65.723 | 1.00 | 38.23 | C |
| ATOM | 2445 | N | THR | C | 121 | −21.011 | 18.383 | 64.202 | 1.00 | 32.90 | C |
| ATOM | 2446 | CA | THR | C | 121 | −19.565 | 18.227 | 64.311 | 1.00 | 32.11 | C |
| ATOM | 2447 | CB | THR | C | 121 | −19.170 | 17.011 | 65.194 | 1.00 | 33.55 | C |
| ATOM | 2448 | OG1 | THR | C | 121 | −19.897 | 17.046 | 66.428 | 1.00 | 39.13 | C |
| ATOM | 2449 | CG2 | THR | C | 121 | −17.686 | 17.053 | 65.523 | 1.00 | 34.12 | C |
| ATOM | 2450 | C | THR | C | 121 | −19.053 | 17.964 | 62.905 | 1.00 | 32.36 | C |
| ATOM | 2451 | O | THR | C | 121 | −19.716 | 17.286 | 62.129 | 1.00 | 34.28 | C |
| ATOM | 2452 | N | ALA | C | 122 | −17.894 | 18.514 | 62.561 | 1.00 | 30.66 | C |
| ATOM | 2453 | CA | ALA | C | 122 | −17.305 | 18.281 | 61.247 | 1.00 | 28.53 | C |
| ATOM | 2454 | CB | ALA | C | 122 | −17.867 | 19.250 | 60.232 | 1.00 | 31.29 | C |
| ATOM | 2455 | C | ALA | C | 122 | −15.806 | 18.458 | 61.374 | 1.00 | 29.78 | C |
| ATOM | 2456 | O | ALA | C | 122 | −15.307 | 18.857 | 62.431 | 1.00 | 31.82 | C |
| ATOM | 2457 | N | ALA | C | 123 | −15.082 | 18.149 | 60.305 | 1.00 | 29.48 | C |
| ATOM | 2458 | CA | ALA | C | 123 | −13.632 | 18.267 | 60.309 | 1.00 | 27.76 | C |
| ATOM | 2459 | CB | ALA | C | 123 | −13.017 | 16.916 | 60.121 | 1.00 | 26.03 | C |
| ATOM | 2460 | C | ALA | C | 123 | −13.143 | 19.203 | 59.218 | 1.00 | 31.35 | C |
| ATOM | 2461 | O | ALA | C | 123 | −13.649 | 19.178 | 58.090 | 1.00 | 31.70 | C |
| ATOM | 2462 | N | MET | C | 124 | −12.160 | 20.034 | 59.556 | 1.00 | 32.01 | C |
| ATOM | 2463 | CA | MET | C | 124 | −11.588 | 20.953 | 58.587 | 1.00 | 31.64 | C |
| ATOM | 2464 | CB | MET | C | 124 | −10.611 | 21.896 | 59.271 | 1.00 | 34.37 | C |
| ATOM | 2465 | CG | MET | C | 124 | −11.250 | 23.078 | 59.947 | 1.00 | 39.95 | C |
| ATOM | 2466 | SD | MET | C | 124 | −11.680 | 24.376 | 58.770 | 1.00 | 39.87 | C |
| ATOM | 2467 | CE | MET | C | 124 | −12.397 | 25.614 | 59.948 | 1.00 | 37.23 | C |
| ATOM | 2468 | C | MET | C | 124 | −10.840 | 20.168 | 57.522 | 1.00 | 32.06 | C |
| ATOM | 2469 | O | MET | C | 124 | −10.089 | 19.236 | 57.822 | 1.00 | 32.54 | C |
| ATOM | 2470 | N | LYS | C | 125 | −11.048 | 20.542 | 56.273 | 1.00 | 32.84 | C |
| ATOM | 2471 | CA | LYS | C | 125 | −10.370 | 19.901 | 55.164 | 1.00 | 34.78 | C |
| ATOM | 2472 | CB | LYS | C | 125 | −11.253 | 18.816 | 54.554 | 1.00 | 37.64 | C |
| ATOM | 2473 | CG | LYS | C | 125 | −10.576 | 17.957 | 53.504 | 1.00 | 45.47 | C |
| ATOM | 2474 | CD | LYS | C | 125 | −11.592 | 16.990 | 52.880 | 1.00 | 55.83 | C |
| ATOM | 2475 | CE | LYS | C | 125 | −10.961 | 16.058 | 51.849 | 1.00 | 60.58 | C |
| ATOM | 2476 | NZ | LYS | C | 125 | −9.988 | 15.100 | 52.466 | 1.00 | 64.33 | C |
| ATOM | 2477 | C | LYS | C | 125 | −10.149 | 21.033 | 54.179 | 1.00 | 34.20 | C |
| ATOM | 2478 | O | LYS | C | 125 | −11.102 | 21.534 | 53.587 | 1.00 | 32.34 | C |
| ATOM | 2479 | N | ASN | C | 126 | −8.898 | 21.451 | 54.023 | 1.00 | 30.46 | C |
| ATOM | 2480 | CA | ASN | C | 126 | −8.589 | 22.544 | 53.125 | 1.00 | 31.96 | C |
| ATOM | 2481 | CB | ASN | C | 126 | −8.947 | 22.178 | 51.684 | 1.00 | 36.86 | C |
| ATOM | 2482 | CG | ASN | C | 126 | −8.154 | 20.993 | 51.173 | 1.00 | 45.11 | C |
| ATOM | 2483 | OD1 | ASN | C | 126 | −6.927 | 20.959 | 51.285 | 1.00 | 48.41 | C |
| ATOM | 2484 | ND2 | ASN | C | 126 | −8.849 | 20.012 | 50.601 | 1.00 | 47.56 | C |
| ATOM | 2485 | C | ASN | C | 126 | −9.338 | 23.811 | 53.537 | 1.00 | 31.04 | C |
| ATOM | 2486 | O | ASN | C | 126 | −10.034 | 24.421 | 52.727 | 1.00 | 29.54 | C |
| ATOM | 2487 | N | GLN | C | 127 | −9.189 | 24.183 | 54.806 | 1.00 | 28.27 | C |
| ATOM | 2488 | CA | GLN | C | 127 | −9.804 | 25.380 | 55.383 | 1.00 | 29.05 | C |
| ATOM | 2489 | CB | GLN | C | 127 | −9.345 | 26.647 | 54.627 | 1.00 | 31.06 | C |
| ATOM | 2490 | CG | GLN | C | 127 | −7.820 | 26.870 | 54.584 | 1.00 | 28.27 | C |
| ATOM | 2491 | CD | GLN | C | 127 | −7.204 | 26.988 | 55.976 | 1.00 | 33.30 | C |
| ATOM | 2492 | OE1 | GLN | C | 127 | −6.353 | 26.188 | 56.353 | 1.00 | 37.54 | C |
| ATOM | 2493 | NE2 | GLN | C | 127 | −7.638 | 27.985 | 56.744 | 1.00 | 34.51 | C |
| ATOM | 2494 | C | GLN | C | 127 | −11.326 | 25.346 | 55.418 | 1.00 | 29.78 | C |
| ATOM | 2495 | O | GLN | C | 127 | −11.958 | 26.327 | 55.810 | 1.00 | 33.17 | C |
| ATOM | 2496 | N | VAL | C | 128 | −11.919 | 24.226 | 55.018 | 1.00 | 27.09 | C |
| ATOM | 2497 | CA | VAL | C | 128 | −13.372 | 24.134 | 55.000 | 1.00 | 26.31 | C |
| ATOM | 2498 | CB | VAL | C | 128 | −13.925 | 24.031 | 53.552 | 1.00 | 26.13 | C |
| ATOM | 2499 | CG1 | VAL | C | 128 | −15.440 | 23.846 | 53.578 | 1.00 | 27.65 | C |
| ATOM | 2500 | CG2 | VAL | C | 128 | −13.553 | 25.279 | 52.763 | 1.00 | 24.52 | C |
| ATOM | 2501 | C | VAL | C | 128 | −13.932 | 22.966 | 55.800 | 1.00 | 28.20 | C |
| ATOM | 2502 | O | VAL | C | 128 | −13.536 | 21.814 | 55.620 | 1.00 | 27.75 | C |
| ATOM | 2503 | N | ALA | C | 129 | −14.861 | 23.272 | 56.692 | 1.00 | 29.62 | C |
| ATOM | 2504 | CA | ALA | C | 129 | −15.492 | 22.232 | 57.497 | 1.00 | 32.84 | C |
| ATOM | 2505 | CB | ALA | C | 129 | −15.454 | 22.604 | 58.987 | 1.00 | 30.86 | C |
| ATOM | 2506 | C | ALA | C | 129 | −16.933 | 22.099 | 56.995 | 1.00 | 32.34 | C |
| ATOM | 2507 | O | ALA | C | 129 | −17.777 | 22.973 | 57.233 | 1.00 | 32.04 | C |
| ATOM | 2508 | N | ARG | C | 130 | −17.193 | 21.009 | 56.281 | 1.00 | 31.86 | C |
| ATOM | 2509 | CA | ARG | C | 130 | −18.507 | 20.751 | 55.719 | 1.00 | 33.45 | C |
| ATOM | 2510 | CB | ARG | C | 130 | −18.361 | 20.040 | 54.379 | 1.00 | 35.33 | C |
| ATOM | 2511 | CG | ARG | C | 130 | −19.665 | 19.826 | 53.676 | 1.00 | 45.75 | C |
| ATOM | 2512 | CD | ARG | C | 130 | −19.479 | 19.090 | 52.362 | 1.00 | 54.06 | C |
| ATOM | 2513 | NE | ARG | C | 130 | −20.756 | 18.917 | 51.664 | 1.00 | 61.23 | C |
| ATOM | 2514 | CZ | ARG | C | 130 | −21.792 | 18.229 | 52.143 | 1.00 | 63.07 | C |
| ATOM | 2515 | NH1 | ARG | C | 130 | −21.712 | 17.637 | 53.333 | 1.00 | 63.02 | C |
| ATOM | 2516 | NH2 | ARG | C | 130 | −22.913 | 18.137 | 51.435 | 1.00 | 64.23 | C |
| ATOM | 2517 | C | ARG | C | 130 | −19.343 | 19.895 | 56.649 | 1.00 | 34.66 | C |
| ATOM | 2518 | O | ARG | C | 130 | −18.976 | 18.760 | 56.944 | 1.00 | 35.29 | C |
| ATOM | 2519 | N | PHE | C | 131 | −20.460 | 20.434 | 57.125 | 1.00 | 34.15 | C |
| ATOM | 2520 | CA | PHE | C | 131 | −21.327 | 19.659 | 58.002 | 1.00 | 33.96 | C |
| ATOM | 2521 | CB | PHE | C | 131 | −22.112 | 20.571 | 58.940 | 1.00 | 32.95 | C |
| ATOM | 2522 | CG | PHE | C | 131 | −21.249 | 21.251 | 59.956 | 1.00 | 31.91 | C |
| ATOM | 2523 | CD1 | PHE | C | 131 | −20.435 | 22.325 | 59.589 | 1.00 | 29.13 | C |
| ATOM | 2524 | CD2 | PHE | C | 131 | −21.199 | 20.781 | 61.268 | 1.00 | 30.24 | C |
| ATOM | 2525 | CE1 | PHE | C | 131 | −19.584 | 22.920 | 60.512 | 1.00 | 31.57 | C |
| ATOM | 2526 | CE2 | PHE | C | 131 | −20.346 | 21.370 | 62.206 | 1.00 | 34.72 | C |
| ATOM | 2527 | CZ | PHE | C | 131 | −19.536 | 22.444 | 61.824 | 1.00 | 33.05 | C |
| ATOM | 2528 | C | PHE | C | 131 | −22.266 | 18.808 | 57.178 | 1.00 | 35.03 | C |
| ATOM | 2529 | O | PHE | C | 131 | −22.851 | 19.265 | 56.209 | 1.00 | 34.67 | C |
| ATOM | 2530 | N | ASN | C | 132 | −22.386 | 17.550 | 57.568 | 1.00 | 41.65 | C |
| ATOM | 2531 | CA | ASN | C | 132 | −23.231 | 16.596 | 56.863 | 1.00 | 41.54 | C |
| ATOM | 2532 | CB | ASN | C | 132 | −22.569 | 15.211 | 56.924 | 1.00 | 44.92 | C |
| ATOM | 2533 | CG | ASN | C | 132 | −23.418 | 14.136 | 56.297 | 1.00 | 53.89 | C |
| ATOM | 2534 | OD1 | ASN | C | 132 | −23.853 | 14.256 | 55.148 | 1.00 | 57.06 | C |
| ATOM | 2535 | ND2 | ASN | C | 132 | −23.663 | 13.070 | 57.047 | 1.00 | 58.25 | C |
| ATOM | 2536 | C | ASN | C | 132 | −24.633 | 16.567 | 57.484 | 1.00 | 40.07 | C |
| ATOM | 2537 | O | ASN | C | 132 | −24.844 | 15.998 | 58.563 | 1.00 | 41.33 | C |
| ATOM | 2538 | N | ASP | C | 133 | −25.574 | 17.205 | 56.798 | 1.00 | 34.50 | C |
| ATOM | 2539 | CA | ASP | C | 133 | −26.962 | 17.288 | 57.234 | 1.00 | 35.58 | C |
| ATOM | 2540 | CB | ASP | C | 133 | −27.636 | 15.906 | 57.173 | 1.00 | 37.21 | C |
| ATOM | 2541 | CG | ASP | C | 133 | −29.171 | 15.987 | 57.256 | 1.00 | 43.90 | C |
| ATOM | 2542 | OD1 | ASP | C | 133 | −29.770 | 16.831 | 56.552 | 1.00 | 43.63 | C |
| ATOM | 2543 | OD2 | ASP | C | 133 | −29.784 | 15.199 | 58.013 | 1.00 | 43.87 | C |
| ATOM | 2544 | C | ASP | C | 133 | −27.130 | 17.878 | 58.627 | 1.00 | 34.54 | C |
| ATOM | 2545 | O | ASP | C | 133 | −27.699 | 17.241 | 59.512 | 1.00 | 33.61 | C |
| ATOM | 2546 | N | LEU | C | 134 | −26.629 | 19.093 | 58.826 | 1.00 | 35.80 | C |
| ATOM | 2547 | CA | LEU | C | 134 | −26.767 | 19.760 | 60.117 | 1.00 | 34.83 | C |
| ATOM | 2548 | CB | LEU | C | 134 | −25.970 | 21.068 | 60.149 | 1.00 | 34.32 | C |
| ATOM | 2549 | CG | LEU | C | 134 | −26.262 | 22.028 | 61.312 | 1.00 | 32.15 | C |
| ATOM | 2550 | CD1 | LEU | C | 134 | −25.800 | 21.417 | 62.604 | 1.00 | 26.87 | C |
| ATOM | 2551 | CD2 | LEU | C | 134 | −25.562 | 23.352 | 61.089 | 1.00 | 30.03 | C |
| ATOM | 2552 | C | LEU | C | 134 | −28.244 | 20.070 | 60.254 | 1.00 | 34.79 | C |
| ATOM | 2553 | O | LEU | C | 134 | −28.860 | 20.599 | 59.332 | 1.00 | 36.02 | C |
| ATOM | 2554 | N | ARG | C | 135 | −28.816 | 19.739 | 61.400 | 1.00 | 34.48 | C |
| ATOM | 2555 | CA | ARG | C | 135 | −30.228 | 19.990 | 61.622 | 1.00 | 31.38 | C |
| ATOM | 2556 | CB | ARG | C | 135 | −31.012 | 18.685 | 61.491 | 1.00 | 34.23 | C |
| ATOM | 2557 | CG | ARG | C | 135 | −32.006 | 18.652 | 60.343 | 1.00 | 39.58 | C |
| ATOM | 2558 | CD | ARG | C | 135 | −32.706 | 17.314 | 60.300 | 1.00 | 39.03 | C |
| ATOM | 2559 | NE | ARG | C | 135 | −31.749 | 16.258 | 60.014 | 1.00 | 44.15 | C |
| ATOM | 2560 | CZ | ARG | C | 135 | −31.725 | 15.080 | 60.622 | 1.00 | 43.28 | C |
| ATOM | 2561 | NH1 | ARG | C | 135 | −32.609 | 14.790 | 61.565 | 1.00 | 42.27 | C |
| ATOM | 2562 | NH2 | ARG | C | 135 | −30.805 | 14.192 | 60.285 | 1.00 | 45.32 | C |
| ATOM | 2563 | C | ARG | C | 135 | −30.482 | 20.584 | 62.992 | 1.00 | 30.74 | C |
| ATOM | 2564 | O | ARG | C | 135 | −29.874 | 20.164 | 63.978 | 1.00 | 32.87 | C |
| ATOM | 2565 | N | PHE | C | 136 | −31.369 | 21.575 | 63.046 | 1.00 | 30.05 | C |
| ATOM | 2566 | CA | PHE | C | 136 | −31.745 | 22.201 | 64.305 | 1.00 | 30.91 | C |
| ATOM | 2567 | CB | PHE | C | 136 | −32.048 | 23.692 | 64.115 | 1.00 | 29.30 | C |
| ATOM | 2568 | CG | PHE | C | 136 | −30.908 | 24.465 | 63.547 | 1.00 | 29.53 | C |
| ATOM | 2569 | CD1 | PHE | C | 136 | −30.594 | 24.372 | 62.199 | 1.00 | 31.18 | C |
| ATOM | 2570 | CD2 | PHE | C | 136 | −30.099 | 25.240 | 64.372 | 1.00 | 29.41 | C |
| ATOM | 2571 | CE1 | PHE | C | 136 | −29.480 | 25.036 | 61.679 | 1.00 | 33.45 | C |
| ATOM | 2572 | CE2 | PHE | C | 136 | −28.993 | 25.905 | 63.867 | 1.00 | 28.24 | C |
| ATOM | 2573 | CZ | PHE | C | 136 | −28.678 | 25.804 | 62.519 | 1.00 | 30.00 | C |
| ATOM | 2574 | C | PHE | C | 136 | −32.996 | 21.470 | 64.783 | 1.00 | 32.66 | C |
| ATOM | 2575 | O | PHE | C | 136 | −34.059 | 21.590 | 64.184 | 1.00 | 33.06 | C |
| ATOM | 2576 | N | VAL | C | 137 | −32.847 | 20.701 | 65.854 | 1.00 | 34.52 | C |
| ATOM | 2577 | CA | VAL | C | 137 | −33.946 | 19.929 | 66.427 | 1.00 | 37.64 | C |
| ATOM | 2578 | CB | VAL | C | 137 | −33.420 | 18.630 | 67.080 | 1.00 | 37.40 | C |
| ATOM | 2579 | CG1 | VAL | C | 137 | −34.560 | 17.881 | 67.750 | 1.00 | 32.20 | C |
| ATOM | 2580 | CG2 | VAL | C | 137 | −32.741 | 17.762 | 66.028 | 1.00 | 30.97 | C |
| ATOM | 2581 | C | VAL | C | 137 | −34.725 | 20.723 | 67.473 | 1.00 | 38.54 | C |
| ATOM | 2582 | O | VAL | C | 137 | −35.950 | 20.694 | 67.486 | 1.00 | 41.88 | C |
| ATOM | 2583 | N | GLY | C | 138 | −34.011 | 21.410 | 68.359 | 1.00 | 39.84 | C |
| ATOM | 2584 | CA | GLY | C | 138 | −34.658 | 22.213 | 69.381 | 1.00 | 37.62 | C |
| ATOM | 2585 | C | GLY | C | 138 | −35.233 | 23.470 | 68.762 | 1.00 | 40.04 | C |
| ATOM | 2586 | O | GLY | C | 138 | −34.643 | 24.061 | 67.867 | 1.00 | 42.09 | C |
| ATOM | 2587 | N | ARG | C | 139 | −36.392 | 23.894 | 69.230 | 1.00 | 44.05 | C |
| ATOM | 2588 | CA | ARG | C | 139 | −37.007 | 25.082 | 68.671 | 1.00 | 47.72 | C |
| ATOM | 2589 | CB | ARG | C | 139 | −38.529 | 24.913 | 68.656 | 1.00 | 52.02 | C |
| ATOM | 2590 | CG | ARG | C | 139 | −39.122 | 24.775 | 70.047 | 1.00 | 62.63 | C |
| ATOM | 2591 | CD | ARG | C | 139 | −40.624 | 24.497 | 70.044 | 1.00 | 68.39 | C |
| ATOM | 2592 | NE | ARG | C | 139 | −40.983 | 23.265 | 69.342 | 1.00 | 74.23 | C |
| ATOM | 2593 | CZ | ARG | C | 139 | −40.317 | 22.116 | 69.428 | 1.00 | 77.66 | C |
| ATOM | 2594 | NH1 | ARG | C | 139 | −39.231 | 22.020 | 70.186 | 1.00 | 80.47 | C |
| ATOM | 2595 | NH2 | ARG | C | 139 | −40.748 | 21.052 | 68.761 | 1.00 | 77.97 | C |
| ATOM | 2596 | C | ARG | C | 139 | −36.622 | 26.312 | 69.479 | 1.00 | 46.26 | C |
| ATOM | 2597 | O | ARG | C | 139 | −36.347 | 26.212 | 70.668 | 1.00 | 44.22 | C |
| ATOM | 2598 | N | SER | C | 140 | −36.574 | 27.467 | 68.826 | 1.00 | 48.51 | C |
| ATOM | 2599 | CA | SER | C | 140 | −36.259 | 28.709 | 69.519 | 1.00 | 51.43 | C |
| ATOM | 2600 | CB | SER | C | 140 | −35.452 | 29.642 | 68.610 | 1.00 | 49.26 | C |
| ATOM | 2601 | OG | SER | C | 140 | −36.145 | 29.940 | 67.411 | 1.00 | 49.43 | C |
| ATOM | 2602 | C | SER | C | 140 | −37.590 | 29.360 | 69.930 | 1.00 | 55.05 | C |
| ATOM | 2603 | O | SER | C | 140 | −37.618 | 30.412 | 70.567 | 1.00 | 55.52 | C |
| ATOM | 2604 | N | GLY | C | 141 | −38.689 | 28.710 | 69.548 | 1.00 | 58.84 | C |
| ATOM | 2605 | CA | GLY | C | 141 | −40.024 | 29.180 | 69.875 | 1.00 | 64.47 | C |
| ATOM | 2606 | C | GLY | C | 141 | −40.358 | 30.628 | 69.561 | 1.00 | 67.78 | C |
| ATOM | 2607 | O | GLY | C | 141 | −39.944 | 31.176 | 68.537 | 1.00 | 69.18 | C |
| ATOM | 2608 | N | ARG | C | 142 | −41.135 | 31.242 | 70.449 | 1.00 | 69.80 | C |
| ATOM | 2609 | CA | ARG | C | 142 | −41.542 | 32.634 | 70.295 | 1.00 | 72.00 | C |
| ATOM | 2610 | CB | ARG | C | 142 | −42.778 | 32.927 | 71.153 | 1.00 | 73.96 | C |
| ATOM | 2611 | CG | ARG | C | 142 | −44.031 | 32.168 | 70.739 | 1.00 | 77.32 | C |
| ATOM | 2612 | CD | ARG | C | 142 | −45.174 | 32.450 | 71.698 | 1.00 | 80.89 | C |
| ATOM | 2613 | NE | ARG | C | 142 | −46.401 | 31.753 | 71.321 | 1.00 | 83.78 | C |
| ATOM | 2614 | CZ | ARG | C | 142 | −47.527 | 31.787 | 72.028 | 1.00 | 85.49 | C |
| ATOM | 2615 | NH1 | ARG | C | 142 | −47.584 | 32.484 | 73.154 | 1.00 | 84.05 | C |
| ATOM | 2616 | NH2 | ARG | C | 142 | −48.598 | 31.125 | 71.609 | 1.00 | 86.25 | C |
| ATOM | 2617 | C | ARG | C | 142 | −40.399 | 33.540 | 70.725 | 1.00 | 71.62 | C |
| ATOM | 2618 | O | ARG | C | 142 | −40.457 | 34.755 | 70.546 | 1.00 | 71.95 | C |
| ATOM | 2619 | N | GLY | C | 143 | −39.362 | 32.932 | 71.295 | 1.00 | 70.85 | C |
| ATOM | 2620 | CA | GLY | C | 143 | −38.207 | 33.685 | 71.747 | 1.00 | 68.52 | C |
| ATOM | 2621 | C | GLY | C | 143 | −37.324 | 34.171 | 70.614 | 1.00 | 67.12 | C |
| ATOM | 2622 | O | GLY | C | 143 | −37.663 | 34.047 | 69.435 | 1.00 | 67.34 | C |
| ATOM | 2623 | N | ALA | C | 144 | −36.177 | 34.732 | 70.972 | 1.00 | 64.79 | C |
| ATOM | 2624 | CA | ALA | C | 144 | −35.252 | 35.245 | 69.974 | 1.00 | 61.89 | C |
| ATOM | 2625 | CB | ALA | C | 144 | −34.090 | 35.957 | 70.666 | 1.00 | 63.07 | C |
| ATOM | 2626 | C | ALA | C | 144 | −34.730 | 34.120 | 69.085 | 1.00 | 58.43 | C |
| ATOM | 2627 | O | ALA | C | 144 | −34.755 | 32.951 | 69.464 | 1.00 | 57.88 | C |
| ATOM | 2628 | N | SER | C | 145 | −34.265 | 34.474 | 67.895 | 1.00 | 54.22 | C |
| ATOM | 2629 | CA | SER | C | 145 | −33.725 | 33.479 | 66.988 | 1.00 | 51.13 | C |
| ATOM | 2630 | CB | SER | C | 145 | −33.501 | 34.089 | 65.602 | 1.00 | 51.71 | C |
| ATOM | 2631 | OG | SER | C | 145 | −32.457 | 35.044 | 65.623 | 1.00 | 53.53 | C |
| ATOM | 2632 | C | SER | C | 145 | −32.396 | 33.008 | 67.583 | 1.00 | 47.91 | C |
| ATOM | 2633 | O | SER | C | 145 | −31.706 | 33.772 | 68.252 | 1.00 | 48.09 | C |
| ATOM | 2634 | N | PHE | C | 146 | −32.045 | 31.751 | 67.347 | 1.00 | 43.89 | C |
| ATOM | 2635 | CA | PHE | C | 146 | −30.808 | 31.192 | 67.879 | 1.00 | 39.89 | C |
| ATOM | 2636 | CB | PHE | C | 146 | −30.653 | 29.718 | 67.466 | 1.00 | 35.86 | C |
| ATOM | 2637 | CG | PHE | C | 146 | −31.558 | 28.769 | 68.205 | 1.00 | 35.09 | C |
| ATOM | 2638 | CD1 | PHE | C | 146 | −32.105 | 27.658 | 67.551 | 1.00 | 33.70 | C |
| ATOM | 2639 | CD2 | PHE | C | 146 | −31.858 | 28.966 | 69.548 | 1.00 | 29.93 | C |
| ATOM | 2640 | CE1 | PHE | C | 146 | −32.937 | 26.757 | 68.225 | 1.00 | 26.60 | C |
| ATOM | 2641 | CE2 | PHE | C | 146 | −32.688 | 28.072 | 70.227 | 1.00 | 31.24 | C |
| ATOM | 2642 | CZ | PHE | C | 146 | −33.227 | 26.962 | 69.557 | 1.00 | 30.73 | C |
| ATOM | 2643 | C | PHE | C | 146 | −29.538 | 31.929 | 67.472 | 1.00 | 38.25 | C |
| ATOM | 2644 | O | PHE | C | 146 | −29.459 | 32.584 | 66.429 | 1.00 | 36.92 | C |
| ATOM | 2645 | N | THR | C | 147 | −28.542 | 31.810 | 68.335 | 1.00 | 37.43 | C |
| ATOM | 2646 | CA | THR | C | 147 | −27.238 | 32.376 | 68.092 | 1.00 | 34.88 | C |
| ATOM | 2647 | CB | THR | C | 147 | −26.690 | 33.113 | 69.321 | 1.00 | 35.55 | C |
| ATOM | 2648 | OG1 | THR | C | 147 | −27.469 | 34.286 | 69.567 | 1.00 | 41.56 | C |
| ATOM | 2649 | CG2 | THR | C | 147 | −25.248 | 33.505 | 69.095 | 1.00 | 37.91 | C |
| ATOM | 2650 | C | THR | C | 147 | −26.383 | 31.146 | 67.852 | 1.00 | 35.30 | C |
| ATOM | 2651 | O | THR | C | 147 | −26.460 | 30.166 | 68.610 | 1.00 | 31.84 | C |
| ATOM | 2652 | N | LEU | C | 148 | −25.597 | 31.174 | 66.784 | 1.00 | 35.02 | C |
| ATOM | 2653 | CA | LEU | C | 148 | −24.728 | 30.056 | 66.499 | 1.00 | 37.28 | C |
| ATOM | 2654 | CB | LEU | C | 148 | −24.467 | 29.920 | 65.006 | 1.00 | 38.81 | C |
| ATOM | 2655 | CG | LEU | C | 148 | −25.489 | 29.251 | 64.106 | 1.00 | 42.52 | C |
| ATOM | 2656 | CD1 | LEU | C | 148 | −24.801 | 28.957 | 62.782 | 1.00 | 44.66 | C |
| ATOM | 2657 | CD2 | LEU | C | 148 | −26.004 | 27.961 | 64.734 | 1.00 | 46.30 | C |
| ATOM | 2658 | C | LEU | C | 148 | −23.392 | 30.274 | 67.188 | 1.00 | 38.96 | C |
| ATOM | 2659 | O | LEU | C | 148 | −22.838 | 31.373 | 67.144 | 1.00 | 40.09 | C |
| ATOM | 2660 | N | THR | C | 149 | −22.891 | 29.231 | 67.841 | 1.00 | 35.31 | C |
| ATOM | 2661 | CA | THR | C | 149 | −21.591 | 29.302 | 68.473 | 1.00 | 31.90 | C |
| ATOM | 2662 | CB | THR | C | 149 | −21.673 | 28.973 | 69.950 | 1.00 | 32.28 | C |
| ATOM | 2663 | OG1 | THR | C | 149 | −22.312 | 30.056 | 70.621 | 1.00 | 33.54 | C |
| ATOM | 2664 | CG2 | THR | C | 149 | −20.288 | 28.793 | 70.537 | 1.00 | 31.41 | C |
| ATOM | 2665 | C | THR | C | 149 | −20.735 | 28.275 | 67.742 | 1.00 | 31.48 | C |
| ATOM | 2666 | O | THR | C | 149 | −20.931 | 27.069 | 67.869 | 1.00 | 32.23 | C |
| ATOM | 2667 | N | ILE | C | 150 | −19.805 | 28.763 | 66.940 | 1.00 | 30.13 | C |
| ATOM | 2668 | CA | ILE | C | 150 | −18.941 | 27.887 | 66.181 | 1.00 | 29.56 | C |
| ATOM | 2669 | CB | ILE | C | 150 | −18.665 | 28.461 | 64.782 | 1.00 | 23.40 | C |
| ATOM | 2670 | CG2 | ILE | C | 150 | −17.695 | 27.579 | 64.043 | 1.00 | 18.98 | C |
| ATOM | 2671 | CG1 | ILE | C | 150 | −19.971 | 28.570 | 64.003 | 1.00 | 23.80 | C |
| ATOM | 2672 | CD1 | ILE | C | 150 | −19.857 | 29.432 | 62.768 | 1.00 | 24.95 | C |
| ATOM | 2673 | C | ILE | C | 150 | −17.634 | 27.748 | 66.927 | 1.00 | 32.46 | C |
| ATOM | 2674 | O | ILE | C | 150 | −16.981 | 28.749 | 67.240 | 1.00 | 30.65 | C |
| ATOM | 2675 | N | THR | C | 151 | −17.255 | 26.508 | 67.218 | 1.00 | 30.98 | C |
| ATOM | 2676 | CA | THR | C | 151 | −16.008 | 26.277 | 67.915 | 1.00 | 30.46 | C |
| ATOM | 2677 | CB | THR | C | 151 | −16.240 | 25.592 | 69.273 | 1.00 | 33.42 | C |
| ATOM | 2678 | OG1 | THR | C | 151 | −17.228 | 26.320 | 70.016 | 1.00 | 34.64 | C |
| ATOM | 2679 | CG2 | THR | C | 151 | −14.952 | 25.575 | 70.071 | 1.00 | 34.36 | C |
| ATOM | 2680 | C | THR | C | 151 | −15.057 | 25.428 | 67.088 | 1.00 | 30.67 | C |
| ATOM | 2681 | O | THR | C | 151 | −15.369 | 24.304 | 66.699 | 1.00 | 32.02 | C |
| ATOM | 2682 | N | VAL | C | 152 | −13.894 | 25.995 | 66.810 | 1.00 | 30.57 | C |
| ATOM | 2683 | CA | VAL | C | 152 | −12.850 | 25.323 | 66.060 | 1.00 | 30.34 | C |
| ATOM | 2684 | CB | VAL | C | 152 | −12.203 | 26.302 | 65.073 | 1.00 | 30.33 | C |
| ATOM | 2685 | CG1 | VAL | C | 152 | −11.000 | 25.663 | 64.420 | 1.00 | 27.83 | C |
| ATOM | 2686 | CG2 | VAL | C | 152 | −13.244 | 26.736 | 64.020 | 1.00 | 28.18 | C |
| ATOM | 2687 | C | VAL | C | 152 | −11.835 | 24.850 | 67.105 | 1.00 | 33.29 | C |
| ATOM | 2688 | O | VAL | C | 152 | −11.215 | 25.664 | 67.796 | 1.00 | 35.90 | C |
| ATOM | 2689 | N | PHE | C | 153 | −11.668 | 23.538 | 67.227 | 1.00 | 33.01 | C |
| ATOM | 2690 | CA | PHE | C | 153 | −10.764 | 23.003 | 68.232 | 1.00 | 31.57 | C |
| ATOM | 2691 | CB | PHE | C | 153 | −11.254 | 21.618 | 68.700 | 1.00 | 28.21 | C |
| ATOM | 2692 | CG | PHE | C | 153 | −12.533 | 21.681 | 69.506 | 1.00 | 30.26 | C |
| ATOM | 2693 | CD1 | PHE | C | 153 | −13.774 | 21.666 | 68.876 | 2.00 | 28.52 | C |
| ATOM | 2694 | CD2 | PHE | C | 153 | −12.491 | 21.884 | 70.887 | 1.00 | 27.37 | C |
| ATOM | 2695 | CE1 | PHE | C | 153 | −14.947 | 21.862 | 69.603 | 1.00 | 29.98 | C |
| ATOM | 2696 | CE2 | PHE | C | 153 | −13.658 | 22.081 | 71.623 | 1.00 | 30.38 | C |
| ATOM | 2697 | CZ | PHE | C | 153 | −14.885 | 22.073 | 70.987 | 1.00 | 29.04 | C |
| ATOM | 2698 | C | PHE | C | 153 | −9.270 | 22.969 | 67.940 | 1.00 | 32.74 | C |
| ATOM | 2699 | O | PHE | C | 153 | −8.679 | 21.907 | 67.752 | 1.00 | 34.62 | C |
| ATOM | 2700 | N | THR | C | 154 | −8.671 | 24.153 | 67.893 | 1.00 | 32.26 | C |
| ATOM | 2701 | CA | THR | C | 154 | −7.228 | 24.294 | 67.729 | 1.00 | 31.96 | C |
| ATOM | 2702 | CB | THR | C | 154 | −6.854 | 25.569 | 66.955 | 1.00 | 29.50 | C |
| ATOM | 2703 | OG1 | THR | C | 154 | −7.674 | 26.650 | 67.409 | 1.00 | 35.13 | C |
| ATOM | 2704 | CG2 | THR | C | 154 | −7.048 | 25.383 | 65.462 | 1.00 | 27.83 | C |
| ATOM | 2705 | C | THR | C | 154 | −6.785 | 24.469 | 69.183 | 1.00 | 34.27 | C |
| ATOM | 2706 | O | THR | C | 154 | −7.594 | 24.304 | 70.102 | 1.00 | 34.83 | C |
| ATOM | 2707 | N | ASN | C | 155 | −5.526 | 24.822 | 69.409 | 1.00 | 38.31 | C |
| ATOM | 2708 | CA | ASN | C | 155 | −5.038 | 25.002 | 70.776 | 1.00 | 40.65 | C |
| ATOM | 2709 | CB | ASN | C | 155 | −3.975 | 23.953 | 71.068 | 1.00 | 45.15 | C |
| ATOM | 2710 | CG | ASN | C | 155 | −4.535 | 22.548 | 71.041 | 1.00 | 53.11 | C |
| ATOM | 2711 | OD1 | ASN | C | 155 | −5.215 | 22.124 | 71.985 | 1.00 | 54.31 | C |
| ATOM | 2712 | ND2 | ASN | C | 155 | −4.272 | 21.816 | 69.947 | 1.00 | 54.44 | C |
| ATOM | 2713 | C | ASN | C | 155 | −4.473 | 26.391 | 71.044 | 1.00 | 37.93 | C |
| ATOM | 2714 | O | ASN | C | 155 | −3.416 | 26.729 | 70.540 | 1.00 | 41.20 | C |
| ATOM | 2715 | N | CPR | C | 156 | −5.195 | 27.233 | 71.807 | 1.00 | 36.86 | C |
| ATOM | 2716 | CD | CPR | C | 156 | −4.704 | 28.577 | 72.174 | 1.00 | 31.25 | C |
| ATOM | 2717 | CA | CPR | C | 156 | −6.498 | 26.973 | 72.430 | 1.00 | 34.34 | C |
| ATOM | 2718 | CB | CPR | C | 156 | −6.603 | 28.103 | 73.445 | 1.00 | 33.09 | C |
| ATOM | 2719 | CG | CPR | C | 156 | −5.929 | 29.238 | 72.724 | 1.00 | 31.00 | C |
| ATOM | 2720 | C | CPR | C | 156 | −7.623 | 27.018 | 71.369 | 1.00 | 36.18 | C |
| ATOM | 2721 | O | CPR | C | 156 | −7.388 | 27.393 | 70.208 | 1.00 | 32.78 | C |
| ATOM | 2722 | N | PRO | C | 157 | −8.859 | 26.652 | 71.755 | 1.00 | 34.60 | C |
| ATOM | 2723 | CD | PRO | C | 157 | −9.348 | 26.256 | 73.082 | 1.00 | 34.64 | C |
| ATOM | 2724 | CA | PRO | C | 157 | −9.958 | 26.670 | 70.789 | 1.00 | 32.99 | C |
| ATOM | 2725 | CB | PRO | C | 157 | −11.154 | 26.157 | 71.598 | 1.00 | 31.57 | C |
| ATOM | 2726 | CG | PRO | C | 157 | −10.531 | 25.402 | 72.726 | 1.00 | 36.98 | C |
| ATOM | 2727 | C | PRO | C | 157 | −10.219 | 28.067 | 70.268 | 1.00 | 31.65 | C |
| ATOM | 2728 | O | PRO | C | 157 | −9.872 | 29.047 | 70.913 | 1.00 | 33.19 | C |
| ATOM | 2729 | N | GLN | C | 158 | −10.826 | 28.149 | 69.092 | 1.00 | 30.63 | C |
| ATOM | 2730 | CA | GLN | C | 158 | −11.199 | 29.436 | 68.525 | 1.00 | 29.88 | C |
| ATOM | 2731 | CB | GLN | C | 158 | −10.587 | 29.608 | 67.134 | 1.00 | 29.95 | C |
| ATOM | 2732 | CG | GLN | C | 158 | −9.059 | 29.679 | 67.149 | 1.00 | 29.44 | C |
| ATOM | 2733 | CD | GLN | C | 158 | −8.469 | 29.844 | 65.759 | 1.00 | 33.68 | C |
| ATOM | 2734 | OE1 | GLN | C | 158 | −8.689 | 30.861 | 65.094 | 1.00 | 31.91 | C |
| ATOM | 2735 | NE2 | GLN | C | 158 | −7.721 | 28.837 | 65.306 | 1.00 | 34.25 | C |
| ATOM | 2736 | C | GLN | C | 158 | −12.731 | 29.384 | 68.465 | 1.00 | 31.04 | C |
| ATOM | 2737 | O | GLN | C | 158 | −13.319 | 28.386 | 68.047 | 1.00 | 30.16 | C |
| ATOM | 2738 | N | VAL | C | 159 | −13.375 | 30.442 | 68.934 | 1.00 | 30.80 | C |
| ATOM | 2739 | CA | VAL | C | 159 | −14.827 | 30.502 | 68.956 | 1.00 | 27.00 | C |
| ATOM | 2740 | CB | VAL | C | 159 | −15.349 | 30.670 | 70.378 | 1.00 | 28.10 | C |
| ATOM | 2741 | CG1 | VAL | C | 159 | −16.872 | 30.717 | 70.370 | 1.00 | 24.62 | C |
| ATOM | 2742 | CG2 | VAL | C | 159 | −14.820 | 29.536 | 71.255 | 1.00 | 29.13 | C |
| ATOM | 2743 | C | VAL | C | 159 | −15.349 | 31.660 | 68.137 | 1.00 | 27.97 | C |
| ATOM | 2744 | O | VAL | C | 159 | −14.871 | 32.792 | 68.251 | 1.00 | 27.57 | C |
| ATOM | 2745 | N | ALA | C | 160 | −16.339 | 31.360 | 67.308 | 1.00 | 26.03 | C |
| ATOM | 2746 | CA | ALA | C | 160 | −16.952 | 32.355 | 66.461 | 1.00 | 26.93 | C |
| ATOM | 2747 | CB | ALA | C | 160 | −16.658 | 32.049 | 65.017 | 1.00 | 25.07 | C |
| ATOM | 2748 | C | ALA | C | 160 | −18.445 | 32.309 | 66.701 | 1.00 | 28.90 | C |
| ATOM | 2749 | O | ALA | C | 160 | −19.041 | 31.242 | 66.692 | 1.00 | 30.05 | C |
| ATOM | 2750 | N | THR | C | 161 | −19.052 | 33.462 | 66.940 | 1.00 | 30.90 | C |
| ATOM | 2751 | CA | THR | C | 161 | −20.489 | 33.499 | 67.140 | 1.00 | 29.65 | C |
| ATOM | 2752 | CB | THR | C | 161 | −20.903 | 34.316 | 68.385 | 1.00 | 27.74 | C |
| ATOM | 2753 | OG1 | THR | C | 161 | −20.343 | 35.627 | 68.313 | 1.00 | 30.29 | C |
| ATOM | 2754 | CG2 | THR | C | 161 | −20.441 | 33.628 | 69.640 | 1.00 | 28.26 | C |
| ATOM | 2755 | C | THR | C | 161 | −21.131 | 34.095 | 65.903 | 1.00 | 28.92 | C |
| ATOM | 2756 | O | THR | C | 161 | −20.467 | 34.728 | 65.084 | 1.00 | 27.33 | C |
| ATOM | 2757 | N | TYR | C | 162 | −22.430 | 33.861 | 65.786 | 1.00 | 30.38 | C |
| ATOM | 2758 | CA | TYR | C | 162 | −23.252 | 34.314 | 64.678 | 1.00 | 32.56 | C |
| ATOM | 2759 | CB | TYR | C | 162 | −23.277 | 33.195 | 63.635 | 1.00 | 36.78 | C |
| ATOM | 2760 | CG | TYR | C | 162 | −24.112 | 33.397 | 62.387 | 1.00 | 45.90 | C |
| ATOM | 2761 | CD1 | TYR | C | 162 | −24.421 | 32.297 | 61.575 | 1.00 | 47.99 | C |
| ATOM | 2762 | CE1 | TYR | C | 162 | −25.162 | 32.434 | 60.411 | 1.00 | 51.64 | C |
| ATOM | 2763 | CD2 | TYR | C | 162 | −24.574 | 34.659 | 61.997 | 1.00 | 46.21 | C |
| ATOM | 2764 | CE2 | TYR | C | 162 | −25.323 | 34.812 | 60.823 | 1.00 | 51.49 | C |
| ATOM | 2765 | CZ | TYR | C | 162 | −25.612 | 33.688 | 60.033 | 1.00 | 54.89 | C |
| ATOM | 2766 | OH | TYR | C | 162 | −26.337 | 33.792 | 58.857 | 1.00 | 55.83 | C |
| ATOM | 2767 | C | TYR | C | 162 | −24.626 | 34.509 | 65.326 | 1.00 | 33.25 | C |
| ATOM | 2768 | O | TYR | C | 162 | −25.388 | 33.548 | 65.468 | 1.00 | 32.90 | C |
| ATOM | 2769 | N | HIS | C | 163 | −24.917 | 35.740 | 65.746 | 1.00 | 31.28 | C |
| ATOM | 2770 | CA | HIS | C | 163 | −26.189 | 36.073 | 66.399 | 1.00 | 34.00 | C |
| ATOM | 2771 | CB | HIS | C | 163 | −26.063 | 37.367 | 67.210 | 1.00 | 36.61 | C |
| ATOM | 2772 | CG | HIS | C | 163 | −25.039 | 37.301 | 68.299 | 1.00 | 37.87 | C |
| ATOM | 2773 | CD2 | HIS | C | 163 | −25.160 | 37.018 | 69.617 | 1.00 | 38.98 | C |
| ATOM | 2774 | ND1 | HIS | C | 163 | −23.690 | 37.452 | 68.062 | 1.00 | 40.16 | C |
| ATOM | 2775 | CE1 | HIS | C | 163 | −23.023 | 37.259 | 69.186 | 1.00 | 41.31 | C |
| ATOM | 2776 | NE2 | HIS | C | 163 | −23.892 | 36.993 | 70.144 | 1.00 | 42.08 | C |
| ATOM | 2777 | C | HIS | C | 163 | −27.336 | 36.224 | 65.412 | 1.00 | 35.43 | C |
| ATOM | 2778 | O | HIS | C | 163 | −27.117 | 36.539 | 64.239 | 1.00 | 33.12 | C |
| ATOM | 2779 | N | ARG | C | 164 | −28.557 | 36.009 | 65.903 | 1.00 | 38.60 | C |
| ATOM | 2780 | CA | ARG | C | 164 | −29.763 | 36.099 | 65.076 | 1.00 | 41.71 | C |
| ATOM | 2781 | CB | ARG | C | 164 | −30.013 | 37.544 | 64.642 | 1.00 | 48.15 | C |
| ATOM | 2782 | CG | ARG | C | 164 | −30.006 | 38.582 | 65.751 | 1.00 | 56.48 | C |
| ATOM | 2783 | CD | ARG | C | 164 | −29.960 | 39.970 | 65.120 | 1.00 | 65.82 | C |
| ATOM | 2784 | NE | ARG | C | 164 | −29.912 | 41.050 | 66.103 | 1.00 | 74.97 | C |
| ATOM | 2785 | CZ | ARG | C | 164 | −29.835 | 42.344 | 65.791 | 1.00 | 76.67 | C |
| ATOM | 2786 | NH1 | ARG | C | 164 | −29.798 | 42.723 | 64.520 | 1.00 | 76.31 | C |
| ATOM | 2787 | NH2 | ARG | C | 164 | −29.799 | 43.261 | 66.750 | 1.00 | 76.70 | C |
| ATOM | 2788 | C | ARG | C | 164 | −29.558 | 35.252 | 63.829 | 1.00 | 38.09 | C |
| ATOM | 2789 | O | ARG | C | 164 | −29.961 | 35.640 | 62.739 | 1.00 | 38.70 | C |
| ATOM | 2790 | N | ALA | C | 165 | −28.924 | 34.100 | 63.990 | 1.00 | 35.94 | C |
| ATOM | 2791 | CA | ALA | C | 165 | −28.632 | 33.243 | 62.852 | 1.00 | 36.52 | C |
| ATOM | 2792 | CB | ALA | C | 165 | −27.610 | 32.180 | 63.244 | 1.00 | 35.18 | C |
| ATOM | 2793 | C | ALA | C | 165 | −29.846 | 32.580 | 62.238 | 1.00 | 36.86 | C |
| ATOM | 2794 | O | ALA | C | 165 | −29.998 | 32.569 | 61.018 | 1.00 | 37.95 | C |
| ATOM | 2795 | N | ILE | C | 166 | −30.718 | 32.038 | 63.077 | 1.00 | 36.60 | C |
| ATOM | 2796 | CA | ILE | C | 166 | −31.880 | 31.343 | 62.564 | 1.00 | 35.26 | C |
| ATOM | 2797 | CB | ILE | C | 166 | −31.431 | 29.976 | 61.994 | 1.00 | 35.79 | C |
| ATOM | 2798 | CG2 | ILE | C | 166 | −30.664 | 29.194 | 63.060 | 1.00 | 31.10 | C |
| ATOM | 2799 | CG1 | ILE | C | 166 | −32.631 | 29.186 | 61.485 | 1.00 | 35.25 | C |
| ATOM | 2800 | CD1 | ILE | C | 166 | −32.245 | 27.865 | 60.856 | 1.00 | 38.70 | C |
| ATOM | 2801 | C | ILE | C | 166 | −33.010 | 31.136 | 63.578 | 1.00 | 35.18 | C |
| ATOM | 2802 | O | ILE | C | 166 | −32.775 | 30.902 | 64.762 | 1.00 | 33.01 | C |
| ATOM | 2803 | N | LYS | C | 167 | −34.245 | 31.248 | 63.100 | 1.00 | 35.63 | C |
| ATOM | 2804 | CA | LYS | C | 167 | −35.417 | 31.032 | 63.941 | 1.00 | 34.27 | C |
| ATOM | 2805 | CB | LYS | C | 167 | −36.522 | 32.039 | 63.607 | 1.00 | 36.54 | C |
| ATOM | 2806 | CG | LYS | C | 167 | −37.760 | 31.875 | 64.465 | 1.00 | 46.00 | C |
| ATOM | 2807 | CD | LYS | C | 167 | −38.946 | 32.704 | 63.975 | 1.00 | 48.30 | C |
| ATOM | 2808 | CE | LYS | C | 167 | −40.159 | 32.455 | 64.873 | 1.00 | 55.36 | C |
| ATOM | 2809 | NZ | LYS | C | 167 | −41.365 | 33.277 | 64.554 | 1.00 | 58.81 | C |
| ATOM | 2810 | C | LYS | C | 167 | −35.879 | 29.603 | 63.639 | 1.00 | 33.90 | C |
| ATOM | 2811 | O | LYS | C | 167 | −35.968 | 29.196 | 62.482 | 1.00 | 27.13 | C |
| ATOM | 2812 | N | ILE | C | 168 | −36.144 | 28.835 | 64.686 | 1.00 | 33.58 | C |
| ATOM | 2813 | CA | ILE | C | 168 | −36.564 | 27.453 | 64.523 | 1.00 | 32.54 | C |
| ATOM | 2814 | CB | ILE | C | 168 | −35.497 | 26.495 | 65.105 | 1.00 | 33.53 | C |
| ATOM | 2815 | CG2 | ILE | C | 168 | −36.015 | 25.050 | 65.086 | 1.00 | 30.35 | C |
| ATOM | 2816 | CG1 | ILE | C | 168 | −34.181 | 26.637 | 64.321 | 1.00 | 32.41 | C |
| ATOM | 2817 | CD1 | ILE | C | 168 | −34.219 | 26.074 | 62.893 | 1.00 | 28.41 | C |
| ATOM | 2818 | C | ILE | C | 168 | −37.890 | 27.223 | 65.236 | 1.00 | 33.87 | C |
| ATOM | 2819 | O | ILE | C | 168 | −37.984 | 27.377 | 66.459 | 1.00 | 35.02 | C |
| ATOM | 2820 | N | THR | C | 169 | −38.917 | 26.865 | 64.475 | 1.00 | 30.22 | C |
| ATOM | 2821 | CA | THR | C | 169 | −40.231 | 26.605 | 65.062 | 1.00 | 31.80 | C |
| ATOM | 2822 | CB | THR | C | 169 | −41.285 | 27.668 | 64.641 | 1.00 | 32.01 | C |
| ATOM | 2823 | OG1 | THR | C | 169 | −41.416 | 27.689 | 63.212 | 1.00 | 34.75 | C |
| ATOM | 2824 | CG2 | THR | C | 169 | −40.879 | 29.050 | 65.129 | 1.00 | 30.40 | C |
| ATOM | 2825 | C | THR | C | 169 | −40.699 | 25.239 | 64.603 | 1.00 | 31.70 | C |
| ATOM | 2826 | O | THR | C | 169 | −40.129 | 24.673 | 63.674 | 1.00 | 29.33 | C |
| ATOM | 2827 | N | VAL | C | 170 | −41.726 | 24.709 | 65.258 | 1.00 | 32.42 | C |
| ATOM | 2828 | CA | VAL | C | 170 | −42.255 | 23.400 | 64.905 | 1.00 | 37.98 | C |
| ATOM | 2829 | CB | VAL | C | 170 | −43.406 | 22.959 | 65.856 | 1.00 | 42.41 | C |
| ATOM | 2830 | CG1 | VAL | C | 170 | −43.865 | 21.553 | 65.503 | 1.00 | 41.99 | C |
| ATOM | 2831 | CG2 | VAL | C | 170 | −42.941 | 22.994 | 67.301 | 1.00 | 44.49 | C |
| ATOM | 2832 | C | VAL | C | 170 | −42.777 | 23.370 | 63.478 | 1.00 | 38.87 | C |
| ATOM | 2833 | O | VAL | C | 170 | −42.601 | 22.382 | 62.773 | 1.00 | 37.76 | C |
| ATOM | 2834 | N | ASP | C | 171 | −43.412 | 24.448 | 63.037 | 1.00 | 42.37 | C |
| ATOM | 2835 | CA | ASP | C | 171 | −43.944 | 24.461 | 61.680 | 1.00 | 46.42 | C |
| ATOM | 2836 | CB | ASP | C | 171 | −45.284 | 25.204 | 61.629 | 1.00 | 47.98 | C |
| ATOM | 2837 | CG | ASP | C | 171 | −46.346 | 24.561 | 62.498 | 1.00 | 53.83 | C |
| ATOM | 2838 | OD1 | ASP | C | 171 | −46.434 | 23.312 | 62.526 | 1.00 | 56.54 | C |
| ATOM | 2839 | OD2 | ASP | C | 171 | −47.107 | 25.311 | 63.146 | 1.00 | 58.56 | C |
| ATOM | 2840 | C | ASP | C | 171 | −42.989 | 25.080 | 60.670 | 1.00 | 46.43 | C |
| ATOM | 2841 | O | ASP | C | 171 | −42.985 | 24.708 | 59.498 | 1.00 | 46.56 | C |
| ATOM | 2842 | N | GLY | C | 172 | −42.174 | 26.021 | 61.132 | 1.00 | 46.92 | C |
| ATOM | 2843 | CA | GLY | C | 172 | −41.252 | 26.685 | 60.236 | 1.00 | 46.93 | C |
| ATOM | 2844 | C | GLY | C | 172 | −42.034 | 27.647 | 59.366 | 1.00 | 48.69 | C |
| ATOM | 2845 | O | GLY | C | 172 | −43.213 | 27.890 | 59.625 | 1.00 | 45.44 | C |
| ATOM | 2846 | N | PRO | C | 173 | −41.411 | 28.219 | 58.329 | 1.00 | 51.27 | C |
| ATOM | 2847 | CD | PRO | C | 173 | −40.021 | 28.034 | 57.878 | 1.00 | 50.98 | C |
| ATOM | 2848 | CA | PRO | C | 173 | −42.120 | 29.154 | 57.454 | 1.00 | 56.17 | C |
| ATOM | 2849 | CB | PRO | C | 173 | −40.989 | 29.761 | 56.628 | 1.00 | 53.10 | C |
| ATOM | 2850 | CG | PRO | C | 173 | −40.054 | 28.617 | 56.480 | 1.00 | 49.25 | C |
| ATOM | 2851 | C | PRO | C | 173 | −43.156 | 28.422 | 56.597 | 1.00 | 62.42 | C |
| ATOM | 2852 | O | PRO | C | 173 | −43.326 | 27.205 | 56.727 | 1.00 | 62.94 | C |
| ATOM | 2853 | N | ARG | C | 174 | −43.835 | 29.166 | 55.725 | 1.00 | 69.93 | C |
| ATOM | 2854 | CA | ARG | C | 174 | −44.858 | 28.604 | 54.837 | 1.00 | 76.32 | C |
| ATOM | 2855 | CB | ARG | C | 174 | −46.159 | 29.399 | 54.968 | 1.00 | 77.16 | C |
| ATOM | 2856 | CG | ARG | C | 174 | −46.554 | 29.715 | 56.399 | 1.00 | 81.72 | C |
| ATOM | 2857 | CD | ARG | C | 174 | −47.685 | 30.734 | 56.444 | 1.00 | 85.13 | C |
| ATOM | 2858 | NE | ARG | C | 174 | −47.872 | 31.265 | 57.792 | 1.00 | 87.59 | C |
| ATOM | 2859 | CZ | ARG | C | 174 | −48.687 | 32.270 | 58.099 | 1.00 | 88.15 | C |
| ATOM | 2860 | NH1 | ARG | C | 174 | −49.405 | 32.866 | 57.154 | 1.00 | 88.50 | C |
| ATOM | 2861 | NH2 | ARG | C | 174 | −48.773 | 32.685 | 59.356 | 1.00 | 87.76 | C |
| ATOM | 2862 | C | ARG | C | 174 | −44.410 | 28.648 | 53.378 | 1.00 | 79.86 | C |
| ATOM | 2863 | O | ARG | C | 174 | −43.251 | 28.933 | 53.083 | 1.00 | 79.65 | C |
| ATOM | 2864 | N | GLU | C | 175 | −45.357 | 28.359 | 52.488 | 1.00 | 85.68 | C |
| ATOM | 2865 | CA | GLU | C | 175 | −45.184 | 28.370 | 51.032 | 1.00 | 91.53 | C |
| ATOM | 2866 | CB | GLU | C | 175 | −45.301 | 29.809 | 50.517 | 1.00 | 92.84 | C |
| ATOM | 2867 | CG | GLU | C | 175 | −46.175 | 30.717 | 51.359 | 1.00 | 96.02 | C |
| ATOM | 2868 | CD | GLU | C | 175 | −45.984 | 32.171 | 50.988 | 1.00 | 98.68 | C |
| ATOM | 2869 | OE1 | GLU | C | 175 | −44.818 | 32.625 | 50.958 | 1.00 | 98.81 | C |
| ATOM | 2870 | OE2 | GLU | C | 175 | −46.992 | 32.862 | 50.732 | 1.00 | 101.11 | C |
| ATOM | 2871 | C | GLU | C | 175 | −43.942 | 27.688 | 50.415 | 1.00 | 95.17 | C |
| ATOM | 2872 | O | GLU | C | 175 | −43.977 | 26.485 | 50.163 | 1.00 | 96.71 | C |
| ATOM | 2873 | N | PRO | C | 176 | −42.836 | 28.434 | 50.165 | 1.00 | 98.10 | C |
| ATOM | 2874 | CD | PRO | C | 176 | −42.500 | 29.767 | 50.713 | 1.00 | 98.53 | C |
| ATOM | 2875 | CA | PRO | C | 176 | −41.631 | 27.835 | 49.569 | 1.00 | 99.90 | C |
| ATOM | 2876 | CB | PRO | C | 176 | −40.512 | 28.568 | 50.289 | 1.00 | 100.80 | C |
| ATOM | 2877 | CG | PRO | C | 176 | −41.050 | 29.962 | 50.284 | 1.00 | 100.77 | C |
| ATOM | 2878 | C | PRO | C | 176 | −41.472 | 26.313 | 49.613 | 1.00 | 101.27 | C |
| ATOM | 2879 | O | PRO | C | 176 | −42.032 | 25.610 | 48.773 | 1.00 | 100.74 | C |
| ATOM | 2880 | N | ARG | C | 177 | −40.692 | 25.817 | 50.574 | 1.00 | 103.49 | C |
| ATOM | 2881 | CA | ARG | C | 177 | −40.448 | 24.379 | 50.749 | 1.00 | 104.61 | C |
| ATOM | 2882 | CB | ARG | C | 177 | −40.209 | 23.695 | 49.395 | 1.00 | 106.93 | C |
| ATOM | 2883 | CG | ARG | C | 177 | −39.238 | 24.421 | 48.479 | 1.00 | 109.34 | C |
| ATOM | 2884 | CD | ARG | C | 177 | −39.044 | 23.652 | 47.184 | 1.00 | 111.71 | C |
| ATOM | 2885 | NE | ARG | C | 177 | −38.327 | 24.434 | 46.180 | 1.00 | 113.33 | C |
| ATOM | 2886 | CZ | ARG | C | 177 | −38.003 | 23.986 | 44.970 | 1.00 | 113.79 | C |
| ATOM | 2887 | NH1 | ARG | C | 177 | −38.327 | 22.752 | 44.605 | 1.00 | 114.52 | C |
| ATOM | 2888 | NH2 | ARG | C | 177 | −37.359 | 24.774 | 44.121 | 1.00 | 113.88 | C |
| ATOM | 2889 | C | ARG | C | 177 | −39.261 | 24.112 | 51.675 | 1.00 | 103.63 | C |
| ATOM | 2890 | O | ARG | C | 177 | −39.271 | 24.525 | 52.834 | 1.00 | 102.56 | C |
| ATOM | 2891 | N | GLY | C | 178 | −38.243 | 23.422 | 51.165 | 1.00 | 102.94 | C |
| ATOM | 2892 | CA | GLY | C | 178 | −37.076 | 23.128 | 51.978 | 1.00 | 102.65 | C |
| ATOM | 2893 | C | GLY | C | 178 | −35.894 | 22.525 | 51.239 | 1.00 | 101.95 | C |
| ATOM | 2894 | OT1 | GLY | C | 178 | −35.417 | 21.448 | 51.659 | 1.00 | 101.09 | C |
| ATOM | 2895 | OT2 | GLY | C | 178 | −35.430 | 23.130 | 50.250 | 1.00 | 101.36 | C |
| ATOM | 2896 | CB | PRO | D | 2 | −5.292 | 30.702 | 65.460 | 1.00 | 52.27 | D |
| ATOM | 2897 | CG | PRO | D | 2 | −4.693 | 29.924 | 66.647 | 1.00 | 48.76 | D |
| ATOM | 2898 | C | PRO | D | 2 | −4.924 | 33.088 | 64.680 | 1.00 | 46.61 | D |
| ATOM | 2899 | O | PRO | D | 2 | −4.346 | 33.598 | 63.717 | 1.00 | 56.06 | D |
| ATOM | 2900 | N | PRO | D | 2 | −3.858 | 32.123 | 66.739 | 1.00 | 47.77 | D |
| ATOM | 2901 | CD | PRO | D | 2 | −4.279 | 31.031 | 67.638 | 1.00 | 45.88 | D |
| ATOM | 2902 | CA | PRO | D | 2 | −4.316 | 31.856 | 65.361 | 1.00 | 45.72 | D |
| ATOM | 2903 | N | ARG | D | 3 | −6.073 | 33.583 | 65.131 | 1.00 | 39.26 | D |
| ATOM | 2904 | CA | ARG | D | 3 | −6.598 | 34.796 | 64.496 | 1.00 | 33.81 | D |
| ATOM | 2905 | CB | ARG | D | 3 | −7.497 | 34.483 | 63.316 | 1.00 | 36.56 | D |
| ATOM | 2906 | CG | ARG | D | 3 | −7.703 | 35.662 | 62.394 | 1.00 | 29.67 | D |
| ATOM | 2907 | CD | ARG | D | 3 | −6.404 | 35.920 | 61.683 | 1.00 | 39.45 | D |
| ATOM | 2908 | NE | ARG | D | 3 | −6.453 | 37.097 | 60.832 | 1.00 | 42.92 | D |
| ATOM | 2909 | CZ | ARG | D | 3 | −7.346 | 37.272 | 59.870 | 1.00 | 45.83 | D |
| ATOM | 2910 | NH1 | ARG | D | 3 | −8.268 | 36.347 | 59.636 | 1.00 | 43.30 | D |
| ATOM | 2911 | NH2 | ARG | D | 3 | −7.316 | 38.376 | 59.144 | 1.00 | 47.57 | D |
| ATOM | 2912 | C | ARG | D | 3 | −7.337 | 35.737 | 65.423 | 1.00 | 32.64 | D |
| ATOM | 2913 | O | ARG | D | 3 | −8.282 | 35.363 | 66.123 | 1.00 | 30.04 | D |
| ATOM | 2914 | N | VAL | D | 4 | −6.916 | 36.990 | 65.381 | 1.00 | 29.44 | D |
| ATOM | 2915 | CA | VAL | D | 4 | −7.480 | 37.999 | 66.244 | 1.00 | 26.58 | D |
| ATOM | 2916 | CB | VAL | D | 4 | −6.520 | 38.227 | 67.414 | 1.00 | 21.86 | D |
| ATOM | 2917 | CG1 | VAL | D | 4 | −6.331 | 36.925 | 68.192 | 1.00 | 13.19 | D |
| ATOM | 2918 | CG2 | VAL | D | 4 | −5.162 | 38.661 | 66.868 | 1.00 | 23.56 | D |
| ATOM | 2919 | C | VAL | D | 4 | −7.673 | 39.298 | 65.482 | 1.00 | 26.33 | D |
| ATOM | 2920 | O | VAL | D | 4 | −7.024 | 39.524 | 64.474 | 1.00 | 28.70 | D |
| ATOM | 2921 | N | VAL | D | 5 | −8.585 | 40.139 | 65.955 | 1.00 | 29.69 | D |
| ATOM | 2922 | CA | VAL | D | 5 | −8.820 | 41.437 | 65.342 | 1.00 | 32.32 | D |
| ATOM | 2923 | CB | VAL | D | 5 | −9.999 | 42.122 | 65.990 | 1.00 | 29.86 | D |
| ATOM | 2924 | CG1 | VAL | D | 5 | −11.249 | 41.280 | 65.822 | 1.00 | 26.92 | D |
| ATOM | 2925 | CG2 | VAL | D | 5 | −9.695 | 42.334 | 67.459 | 1.00 | 29.50 | D |
| ATOM | 2926 | C | VAL | D | 5 | −7.545 | 42.206 | 65.688 | 1.00 | 37.11 | D |
| ATOM | 2927 | O | VAL | D | 5 | −6.834 | 41.809 | 66.603 | 1.00 | 38.36 | D |
| ATOM | 2928 | N | PRO | D | 6 | −7.232 | 43.299 | 64.965 | 1.00 | 40.76 | D |
| ATOM | 2929 | CD | PRO | D | 6 | −7.927 | 43.843 | 63.788 | 1.00 | 41.67 | D |
| ATOM | 2930 | CA | PRO | D | 6 | −6.010 | 44.072 | 65.252 | 1.00 | 44.89 | D |
| ATOM | 2931 | CB | PRO | D | 6 | −6.004 | 45.145 | 64.162 | 1.00 | 43.45 | D |
| ATOM | 2932 | CG | PRO | D | 6 | −7.444 | 45.267 | 63.774 | 1.00 | 46.28 | D |
| ATOM | 2933 | C | PRO | D | 6 | −5.858 | 44.661 | 66.663 | 1.00 | 48.66 | D |
| ATOM | 2934 | O | PRO | D | 6 | −4.759 | 44.655 | 67.226 | 1.00 | 50.43 | D |
| ATOM | 2935 | N | ASP | D | 7 | −6.950 | 45.155 | 67.241 | 1.00 | 49.36 | D |
| ATOM | 2936 | CA | ASP | D | 7 | −6.888 | 45.721 | 68.581 | 1.00 | 48.13 | D |
| ATOM | 2937 | CB | ASP | D | 7 | −7.271 | 47.199 | 68.514 | 1.00 | 53.05 | D |
| ATOM | 2938 | CG | ASP | D | 7 | −7.185 | 47.897 | 69.860 | 1.00 | 58.19 | D |
| ATOM | 2939 | OD1 | ASP | D | 7 | −6.548 | 47.357 | 70.795 | 1.00 | 55.76 | D |
| ATOM | 2940 | OD2 | ASP | D | 7 | −7.753 | 49.006 | 69.968 | 1.00 | 61.95 | D |
| ATOM | 2941 | C | ASP | D | 7 | −7.809 | 44.952 | 69.537 | 1.00 | 47.09 | D |
| ATOM | 2942 | O | ASP | D | 7 | −8.892 | 45.426 | 69.909 | 1.00 | 47.05 | D |
| ATOM | 2943 | N | GLN | D | 8 | −7.362 | 43.761 | 69.931 | 1.00 | 42.57 | D |
| ATOM | 2944 | CA | GLN | D | 8 | −8.124 | 42.897 | 70.824 | 1.00 | 42.76 | D |
| ATOM | 2945 | CB | GLN | D | 8 | −7.289 | 41.691 | 71.259 | 1.00 | 39.32 | D |
| ATOM | 2946 | CG | GLN | D | 8 | −7.027 | 40.687 | 70.164 | 1.00 | 35.60 | D |
| ATOM | 2947 | CD | GLN | D | 8 | −6.088 | 39.592 | 70.607 | 1.00 | 41.78 | D |
| ATOM | 2948 | OE1 | GLN | D | 8 | −6.366 | 38.852 | 71.567 | 1.00 | 42.74 | D |
| ATOM | 2949 | NE2 | GLN | D | 8 | −4.956 | 39.479 | 69.917 | 1.00 | 39.94 | D |
| ATOM | 2950 | C | GLN | D | 8 | −8.662 | 43.590 | 72.061 | 1.00 | 45.21 | D |
| ATOM | 2951 | O | GLN | D | 8 | −9.877 | 43.731 | 72.206 | 1.00 | 49.07 | D |
| ATOM | 2952 | N | ARG | D | 9 | −7.767 | 44.018 | 72.952 | 1.00 | 47.40 | D |
| ATOM | 2953 | CA | ARG | D | 9 | −8.170 | 44.678 | 74.199 | 1.00 | 47.57 | D |
| ATOM | 2954 | CB | ARG | D | 9 | −6.955 | 45.275 | 74.916 | 1.00 | 47.64 | D |
| ATOM | 2955 | CG | ARG | D | 9 | −7.276 | 46.465 | 75.817 | 1.00 | 51.19 | D |
| ATOM | 2956 | CD | ARG | D | 9 | −6.061 | 46.916 | 76.643 | 1.00 | 48.34 | D |
| ATOM | 2957 | NE | ARG | D | 9 | −6.061 | 46.283 | 77.955 | 1.00 | 48.89 | D |
| ATOM | 2958 | CZ | ARG | D | 9 | −5.070 | 45.539 | 78.437 | 1.00 | 54.12 | D |
| ATOM | 2959 | NH1 | ARG | D | 9 | −3.967 | 45.321 | 77.727 | 1.00 | 55.02 | D |
| ATOM | 2960 | NH2 | ARG | D | 9 | −5.197 | 44.983 | 79.630 | 1.00 | 54.27 | D |
| ATOM | 2961 | C | ARG | D | 9 | −9.242 | 45.747 | 74.033 | 1.00 | 45.82 | D |
| ATOM | 2962 | O | ARG | D | 9 | −10.171 | 45.821 | 74.837 | 1.00 | 46.48 | D |
| ATOM | 2963 | N | SER | D | 10 | −9.138 | 46.565 | 72.994 | 1.00 | 42.59 | D |
| ATOM | 2964 | CA | SER | D | 10 | −10.140 | 47.601 | 72.806 | 1.00 | 44.38 | D |
| ATOM | 2965 | CB | SER | D | 10 | −9.712 | 48.576 | 71.719 | 1.00 | 44.30 | D |
| ATOM | 2966 | OG | SER | D | 10 | −10.715 | 49.556 | 71.529 | 1.00 | 48.10 | D |
| ATOM | 2967 | C | SER | D | 10 | −11.517 | 47.030 | 72.475 | 1.00 | 44.71 | D |
| ATOM | 2968 | O | SER | D | 10 | −12.541 | 47.576 | 72.890 | 1.00 | 41.68 | D |
| ATOM | 2969 | N | LYS | D | 11 | −11.554 | 45.939 | 71.715 | 1.00 | 45.68 | D |
| ATOM | 2970 | CA | LYS | D | 11 | −12.835 | 45.338 | 71.382 | 1.00 | 42.24 | D |
| ATOM | 2971 | CB | LYS | D | 11 | −12.658 | 44.259 | 70.324 | 1.00 | 40.36 | D |
| ATOM | 2972 | CG | LYS | D | 11 | −13.959 | 43.605 | 69.938 | 1.00 | 39.06 | D |
| ATOM | 2973 | CD | LYS | D | 11 | −13.795 | 42.642 | 68.777 | 1.00 | 40.91 | D |
| ATOM | 2974 | CE | LYS | D | 11 | −15.152 | 42.111 | 68.367 | 1.00 | 38.35 | D |
| ATOM | 2975 | NZ | LYS | D | 11 | −15.035 | 41.130 | 67.279 | 1.00 | 37.77 | D |
| ATOM | 2976 | C | LYS | D | 11 | −13.444 | 44.739 | 72.655 | 1.00 | 40.49 | D |
| ATOM | 2977 | O | LYS | D | 11 | −14.638 | 44.892 | 72.914 | 1.00 | 41.05 | D |
| ATOM | 2978 | N | PHE | D | 12 | −12.609 | 44.076 | 73.450 | 1.00 | 38.47 | D |
| ATOM | 2979 | CA | PHE | D | 12 | −13.037 | 43.458 | 74.705 | 1.00 | 41.22 | D |
| ATOM | 2980 | CB | PHE | D | 12 | −11.866 | 42.701 | 75.336 | 1.00 | 40.33 | D |
| ATOM | 2981 | CG | PHE | D | 12 | −12.244 | 41.904 | 76.552 | 1.00 | 41.28 | D |
| ATOM | 2982 | CD1 | PHE | D | 12 | −12.565 | 40.557 | 76.441 | 1.00 | 39.90 | D |
| ATOM | 2983 | CD2 | PHE | D | 12 | −12.304 | 42.511 | 77.810 | 1.00 | 41.55 | D |
| ATOM | 2984 | CE1 | PHE | D | 12 | −12.944 | 39.820 | 77.565 | 1.00 | 40.16 | D |
| ATOM | 2985 | CE2 | PHE | D | 12 | −12.679 | 41.789 | 78.936 | 1.00 | 38.30 | D |
| ATOM | 2986 | CZ | PHE | D | 12 | −13.000 | 40.440 | 78.814 | 1.00 | 42.29 | D |
| ATOM | 2987 | C | PHE | D | 12 | −13.572 | 44.478 | 75.719 | 1.00 | 41.82 | D |
| ATOM | 2988 | O | PHE | D | 12 | −14.441 | 44.162 | 76.534 | 1.00 | 40.78 | D |
| ATOM | 2989 | N | GLU | D | 13 | −13.044 | 45.697 | 75.671 | 1.00 | 45.34 | D |
| ATOM | 2990 | CA | GLU | D | 13 | −13.464 | 46.749 | 76.592 | 1.00 | 47.95 | D |
| ATOM | 2991 | CB | GLU | D | 13 | −12.305 | 47.714 | 76.873 | 1.00 | 51.79 | D |
| ATOM | 2992 | CG | GLU | D | 13 | −11.192 | 47.150 | 77.756 | 1.00 | 56.27 | D |
| ATOM | 2993 | CD | GLU | D | 13 | −10.047 | 48.138 | 77.976 | 1.00 | 58.98 | D |
| ATOM | 2994 | OE1 | GLU | D | 13 | −10.107 | 49.267 | 77.436 | 1.00 | 56.25 | D |
| ATOM | 2995 | OE2 | GLU | D | 13 | −9.086 | 47.776 | 78.692 | 1.00 | 59.82 | D |
| ATOM | 2996 | C | GLU | D | 13 | −14.646 | 47.557 | 76.089 | 1.00 | 47.78 | D |
| ATOM | 2997 | O | GLU | D | 13 | −15.429 | 48.075 | 76.887 | 1.00 | 46.36 | D |
| ATOM | 2998 | N | ASN | D | 14 | −14.790 | 47.638 | 74.768 | 1.00 | 47.49 | D |
| ATOM | 2999 | CA | ASN | D | 14 | −15.849 | 48.442 | 74.188 | 1.00 | 45.52 | D |
| ATOM | 3000 | CB | ASN | D | 14 | −15.239 | 49.417 | 73.171 | 1.00 | 48.00 | D |
| ATOM | 3001 | CG | ASN | D | 14 | −14.030 | 50.160 | 73.727 | 1.00 | 49.24 | D |
| ATOM | 3002 | OD1 | ASN | D | 14 | −14.017 | 50.552 | 74.889 | 1.00 | 49.07 | D |
| ATOM | 3003 | ND2 | ASN | D | 14 | −13.012 | 50.362 | 72.893 | 1.00 | 49.54 | D |
| ATOM | 3004 | C | ASN | D | 14 | −17.061 | 47.765 | 73.564 | 1.00 | 46.07 | D |
| ATOM | 3005 | O | ASN | D | 14 | −18.084 | 48.427 | 73.391 | 1.00 | 46.79 | D |
| ATOM | 3006 | N | GLU | D | 15 | −16.983 | 46.482 | 73.208 | 1.00 | 46.51 | D |
| ATOM | 3007 | CA | GLU | D | 15 | −18.158 | 45.834 | 72.608 | 1.00 | 45.54 | D |
| ATOM | 3008 | CB | GLU | D | 15 | −17.756 | 44.757 | 71.589 | 1.00 | 51.10 | D |
| ATOM | 3009 | CG | GLU | D | 15 | −18.848 | 44.407 | 70.513 | 1.00 | 54.30 | D |
| ATOM | 3010 | CD | GLU | D | 15 | −20.321 | 44.511 | 71.010 | 1.00 | 59.14 | D |
| ATOM | 3011 | OE1 | GLU | D | 15 | −20.793 | 45.644 | 71.287 | 1.00 | 63.53 | D |
| ATOM | 3012 | OE2 | GLU | D | 15 | −21.015 | 43.473 | 71.120 | 1.00 | 52.39 | D |
| ATOM | 3013 | C | GLU | D | 15 | −19.070 | 45.205 | 73.663 | 1.00 | 44.72 | D |
| ATOM | 3014 | O | GLU | D | 15 | −18.625 | 44.440 | 74.521 | 1.00 | 43.99 | D |
| ATOM | 3015 | N | GLU | D | 16 | −20.350 | 45.544 | 73.586 | 1.00 | 43.25 | D |
| ATOM | 3016 | CA | GLU | D | 16 | −21.359 | 45.029 | 74.496 | 1.00 | 47.57 | D |
| ATOM | 3017 | CB | GLU | D | 16 | −22.751 | 45.394 | 73.957 | 1.00 | 53.03 | D |
| ATOM | 3018 | CG | GLU | D | 16 | −23.917 | 44.813 | 74.750 | 1.00 | 63.96 | D |
| ATOM | 3019 | CD | GLU | D | 16 | −25.280 | 45.085 | 74.108 | 1.00 | 72.90 | D |
| ATOM | 3020 | OE1 | GLU | D | 16 | −25.487 | 44.702 | 72.928 | 1.00 | 73.75 | D |
| ATOM | 3021 | OE2 | GLU | D | 16 | −26.147 | 45.679 | 74.793 | 1.00 | 76.33 | D |
| ATOM | 3022 | C | GLU | D | 16 | −21.246 | 43.507 | 74.696 | 1.00 | 46.41 | D |
| ATOM | 3023 | O | GLU | D | 16 | −21.428 | 42.995 | 75.803 | 1.00 | 44.71 | D |
| ATOM | 3024 | N | PHE | D | 17 | −20.935 | 42.784 | 73.626 | 1.00 | 45.23 | D |
| ATOM | 3025 | CA | PHE | D | 17 | −20.824 | 41.342 | 73.731 | 1.00 | 43.26 | D |
| ATOM | 3026 | CB | PHE | D | 17 | −20.390 | 40.723 | 72.400 | 1.00 | 41.80 | D |
| ATOM | 3027 | CG | PHE | D | 17 | −20.207 | 39.237 | 72.469 | 1.00 | 38.73 | D |
| ATOM | 3028 | CD1 | PHE | D | 17 | −21.305 | 38.397 | 72.541 | 1.00 | 36.31 | D |
| ATOM | 3029 | CD2 | PHE | D | 17 | −18.930 | 38.683 | 72.526 | 1.00 | 38.69 | D |
| ATOM | 3030 | CE1 | PHE | D | 17 | −21.144 | 37.013 | 72.674 | 1.00 | 36.88 | D |
| ATOM | 3031 | CE2 | PHE | D | 17 | −18.753 | 37.306 | 72.658 | 1.00 | 39.55 | D |
| ATOM | 3032 | CZ | PHE | D | 17 | −19.871 | 36.466 | 72.733 | 1.00 | 39.32 | D |
| ATOM | 3033 | C | PHE | D | 17 | −19.850 | 40.910 | 74.827 | 1.00 | 43.33 | D |
| ATOM | 3034 | O | PHE | D | 17 | −20.133 | 39.968 | 75.565 | 1.00 | 44.76 | D |
| ATOM | 3035 | N | PHE | D | 18 | −18.703 | 41.579 | 74.925 | 1.00 | 41.85 | D |
| ATOM | 3036 | CA | PHE | D | 18 | −17.712 | 41.229 | 75.942 | 1.00 | 39.93 | D |
| ATOM | 3037 | CB | PHE | D | 18 | −16.290 | 41.551 | 75.459 | 1.00 | 39.83 | D |
| ATOM | 3038 | CG | PHE | D | 18 | −15.816 | 40.680 | 74.336 | 1.00 | 33.33 | D |
| ATOM | 3039 | CD1 | PHE | D | 18 | −15.962 | 41.086 | 73.019 | 1.00 | 30.99 | D |
| ATOM | 3040 | CD2 | PHE | D | 18 | −15.254 | 39.438 | 74.602 | 1.00 | 29.30 | D |
| ATOM | 3041 | CE1 | PHE | D | 18 | −15.560 | 40.271 | 71.972 | 1.00 | 31.50 | D |
| ATOM | 3042 | CE2 | PHE | D | 18 | −14.845 | 38.611 | 73.572 | 1.00 | 32.58 | D |
| ATOM | 3043 | CZ | PHE | D | 18 | −15.000 | 39.030 | 72.244 | 1.00 | 34.37 | D |
| ATOM | 3044 | C | PHE | D | 18 | −17.950 | 41.930 | 77.276 | 1.00 | 40.89 | D |
| ATOM | 3045 | O | PHE | D | 18 | −17.563 | 41.423 | 78.330 | 1.00 | 35.27 | D |
| ATOM | 3046 | N | ARG | D | 19 | −18.584 | 43.097 | 77.228 | 1.00 | 44.33 | D |
| ATOM | 3047 | CA | ARG | D | 19 | −18.852 | 43.845 | 78.447 | 1.00 | 48.14 | D |
| ATOM | 3048 | CB | ARG | D | 19 | −19.381 | 45.243 | 78.116 | 1.00 | 52.14 | D |
| ATOM | 3049 | CG | ARG | D | 19 | −18.485 | 46.007 | 77.150 | 1.00 | 60.23 | D |
| ATOM | 3050 | CD | ARG | D | 19 | −18.893 | 47.456 | 76.987 | 1.00 | 62.72 | D |
| ATOM | 3051 | NE | ARG | D | 19 | −18.406 | 48.277 | 78.087 | 1.00 | 70.79 | D |
| ATOM | 3052 | CZ | ARG | D | 19 | −18.412 | 49.606 | 78.083 | 1.00 | 74.77 | D |
| ATOM | 3053 | NH1 | ARG | D | 19 | −18.884 | 50.268 | 77.031 | 1.00 | 74.83 | D |
| ATOM | 3054 | NH2 | ARG | D | 19 | −17.939 | 50.275 | 79.126 | 1.00 | 76.79 | D |
| ATOM | 3055 | C | ARG | D | 19 | −19.858 | 43.104 | 79.300 | 1.00 | 49.93 | D |
| ATOM | 3056 | O | ARG | D | 19 | −19.628 | 42.884 | 80.494 | 1.00 | 50.25 | D |
| ATOM | 3057 | N | LYS | D | 20 | −20.971 | 42.709 | 78.683 | 1.00 | 49.87 | D |
| ATOM | 3058 | CA | LYS | D | 20 | −22.023 | 41.990 | 79.397 | 1.00 | 49.53 | D |
| ATOM | 3059 | CB | LYS | D | 20 | −23.183 | 41.673 | 78.447 | 1.00 | 51.70 | D |
| ATOM | 3060 | CG | LYS | D | 20 | −23.710 | 42.891 | 77.700 | 1.00 | 56.47 | D |
| ATOM | 3061 | CD | LYS | D | 20 | −24.831 | 42.527 | 76.742 | 1.00 | 61.12 | D |
| ATOM | 3062 | CE | LYS | D | 20 | −26.120 | 42.211 | 77.480 | 1.00 | 66.78 | D |
| ATOM | 3063 | NZ | LYS | D | 20 | −27.223 | 41.804 | 76.537 | 1.00 | 69.12 | D |
| ATOM | 3064 | C | LYS | D | 20 | −21.485 | 40.703 | 80.010 | 1.00 | 46.66 | D |
| ATOM | 3065 | O | LYS | D | 20 | −22.091 | 40.131 | 80.911 | 1.00 | 47.82 | D |
| ATOM | 3066 | N | LEU | D | 21 | −20.332 | 40.264 | 79.523 | 1.00 | 43.33 | D |
| ATOM | 3067 | CA | LEU | D | 21 | −19.706 | 39.042 | 80.006 | 1.00 | 44.59 | D |
| ATOM | 3068 | CB | LEU | D | 21 | −19.371 | 38.133 | 78.821 | 1.00 | 42.13 | D |
| ATOM | 3069 | CG | LEU | D | 21 | −20.404 | 37.154 | 78.271 | 1.00 | 39.84 | D |
| ATOM | 3070 | CD1 | LEU | D | 21 | −21.794 | 37.732 | 78.362 | 1.00 | 36.89 | D |
| ATOM | 3071 | CD2 | LEU | D | 21 | −20.020 | 36.808 | 76.822 | 1.00 | 35.53 | D |
| ATOM | 3072 | C | LEU | D | 21 | −18.429 | 39.286 | 80.803 | 1.00 | 44.92 | D |
| ATOM | 3073 | O | LEU | D | 21 | −17.836 | 38.342 | 81.320 | 1.00 | 43.01 | D |
| ATOM | 3074 | N | SER | D | 22 | −18.005 | 40.542 | 80.907 | 1.00 | 47.78 | D |
| ATOM | 3075 | CA | SER | D | 22 | −16.767 | 40.867 | 81.614 | 1.00 | 50.52 | D |
| ATOM | 3076 | CB | SER | D | 22 | −16.248 | 42.226 | 81.136 | 1.00 | 47.09 | D |
| ATOM | 3077 | OG | SER | D | 22 | −17.260 | 43.210 | 81.208 | 1.00 | 53.22 | D |
| ATOM | 3078 | C | SER | D | 22 | −16.823 | 40.820 | 83.152 | 1.00 | 52.28 | D |
| ATOM | 3079 | O | SER | D | 22 | −15.811 | 40.540 | 83.801 | 1.00 | 50.04 | D |
| ATOM | 3080 | N | ARG | D | 23 | −17.994 | 41.077 | 83.730 | 1.00 | 54.15 | D |
| ATOM | 3081 | CA | ARG | D | 23 | −18.154 | 41.041 | 85.186 | 1.00 | 56.17 | D |
| ATOM | 3082 | CB | ARG | D | 23 | −18.977 | 42.243 | 85.674 | 1.00 | 60.08 | D |
| ATOM | 3083 | CG | ARG | D | 23 | −18.374 | 43.591 | 85.325 | 1.00 | 67.23 | D |
| ATOM | 3084 | CD | ARG | D | 23 | −19.301 | 44.759 | 85.659 | 1.00 | 71.52 | D |
| ATOM | 3085 | NE | ARG | D | 23 | −18.689 | 46.031 | 85.266 | 1.00 | 78.09 | D |
| ATOM | 3086 | CZ | ARG | D | 23 | −19.283 | 47.224 | 85.343 | 1.00 | 80.82 | D |
| ATOM | 3087 | NH1 | ARG | D | 23 | −20.528 | 47.329 | 85.811 | 1.00 | 80.78 | D |
| ATOM | 3088 | NH2 | ARG | D | 23 | −18.632 | 48.318 | 84.938 | 1.00 | 79.53 | D |
| ATOM | 3089 | C | ARG | D | 23 | −18.878 | 39.757 | 85.565 | 1.00 | 56.69 | D |
| ATOM | 3090 | O | ARG | D | 23 | −19.770 | 39.307 | 84.841 | 1.00 | 54.95 | D |
| ATOM | 3091 | N | GLU | D | 24 | −18.504 | 39.181 | 86.704 | 1.00 | 57.24 | D |
| ATOM | 3092 | CA | GLU | D | 24 | −19.115 | 37.944 | 87.182 | 1.00 | 57.36 | D |
| ATOM | 3093 | CB | GLU | D | 24 | −18.783 | 37.711 | 88.654 | 1.00 | 60.21 | D |
| ATOM | 3094 | CG | GLU | D | 24 | −17.567 | 36.850 | 88.894 | 1.00 | 65.20 | D |
| ATOM | 3095 | CD | GLU | D | 24 | −17.472 | 36.401 | 90.339 | 1.00 | 69.91 | D |
| ATOM | 3096 | OE1 | GLU | D | 24 | −18.460 | 35.820 | 90.850 | 1.00 | 70.73 | D |
| ATOM | 3097 | OE2 | GLU | D | 24 | −16.411 | 36.626 | 90.962 | 1.00 | 71.73 | D |
| ATOM | 3098 | C | GLU | D | 24 | −20.623 | 37.890 | 87.025 | 1.00 | 56.11 | D |
| ATOM | 3099 | O | GLU | D | 24 | −21.332 | 38.802 | 87.439 | 1.00 | 55.94 | D |
| ATOM | 3100 | N | CYS | D | 25 | −21.105 | 36.808 | 86.424 | 1.00 | 54.18 | D |
| ATOM | 3101 | CA | CYS | D | 25 | −22.533 | 36.608 | 86.231 | 1.00 | 51.71 | D |
| ATOM | 3102 | CB | CYS | D | 25 | −23.011 | 37.255 | 84.922 | 1.00 | 50.84 | D |
| ATOM | 3103 | SG | CYS | D | 25 | −21.986 | 36.944 | 83.468 | 1.00 | 54.31 | D |
| ATOM | 3104 | C | CYS | D | 25 | −22.858 | 35.120 | 86.251 | 1.00 | 50.75 | D |
| ATOM | 3105 | O | CYS | D | 25 | −21.960 | 34.273 | 86.186 | 1.00 | 48.53 | D |
| ATOM | 3106 | N | GLU | D | 26 | −24.143 | 34.805 | 86.366 | 1.00 | 50.87 | D |
| ATOM | 3107 | CA | GLU | D | 26 | −24.575 | 33.417 | 86.413 | 1.00 | 49.98 | D |
| ATOM | 3108 | CB | GLU | D | 26 | −26.082 | 33.313 | 86.634 | 1.00 | 51.88 | D |
| ATOM | 3109 | CG | GLU | D | 26 | −26.538 | 33.647 | 88.031 | 1.00 | 59.28 | D |
| ATOM | 3110 | CD | GLU | D | 26 | −27.982 | 33.243 | 88.267 | 1.00 | 61.03 | D |
| ATOM | 3111 | OE1 | GLU | D | 26 | −28.259 | 32.027 | 88.407 | 1.00 | 64.07 | D |
| ATOM | 3112 | OE2 | GLU | D | 26 | −28.841 | 34.144 | 88.296 | 1.00 | 61.96 | D |
| ATOM | 3113 | C | GLU | D | 26 | −24.233 | 32.613 | 85.177 | 1.00 | 47.31 | D |
| ATOM | 3114 | O | GLU | D | 26 | −24.487 | 33.028 | 84.041 | 1.00 | 46.59 | D |
| ATOM | 3115 | N | ILE | D | 27 | −23.654 | 31.447 | 85.423 | 1.00 | 42.80 | D |
| ATOM | 3116 | CA | ILE | D | 27 | −23.310 | 30.529 | 84.367 | 1.00 | 40.60 | D |
| ATOM | 3117 | CB | ILE | D | 27 | −21.795 | 30.240 | 84.333 | 1.00 | 36.76 | D |
| ATOM | 3118 | CG2 | ILE | D | 27 | −21.040 | 31.494 | 83.916 | 1.00 | 35.57 | D |
| ATOM | 3119 | CG1 | ILE | D | 27 | −21.314 | 29.755 | 85.696 | 1.00 | 32.02 | D |
| ATOM | 3120 | CD1 | ILE | D | 27 | −19.840 | 29.446 | 85.732 | 1.00 | 27.59 | D |
| ATOM | 3121 | C | ILE | D | 27 | −24.101 | 29.266 | 84.677 | 1.00 | 42.35 | D |
| ATOM | 3122 | O | ILE | D | 27 | −24.586 | 29.081 | 85.795 | 1.00 | 43.91 | D |
| ATOM | 3123 | N | LYS | D | 28 | −24.255 | 28.405 | 83.683 | 1.00 | 44.48 | D |
| ATOM | 3124 | CA | LYS | D | 28 | −25.009 | 27.176 | 83.871 | 1.00 | 42.17 | D |
| ATOM | 3125 | CB | LYS | D | 28 | −26.498 | 27.456 | 83.664 | 1.00 | 42.77 | D |
| ATOM | 3126 | CG | LYS | D | 28 | −27.331 | 26.232 | 83.390 | 1.00 | 48.48 | D |
| ATOM | 3127 | CD | LYS | D | 28 | −28.763 | 26.594 | 83.024 | 1.00 | 52.10 | D |
| ATOM | 3128 | CE | LYS | D | 28 | −29.520 | 27.113 | 84.226 | 1.00 | 60.27 | D |
| ATOM | 3129 | NZ | LYS | D | 28 | −30.961 | 27.340 | 83.902 | 1.00 | 67.28 | D |
| ATOM | 3130 | C | LYS | D | 28 | −24.547 | 26.088 | 82.913 | 1.00 | 39.65 | D |
| ATOM | 3131 | O | LYS | D | 28 | −24.371 | 26.331 | 81.715 | 1.00 | 34.84 | D |
| ATOM | 3132 | N | TYR | D | 29 | −24.329 | 24.893 | 83.457 | 1.00 | 38.08 | D |
| ATOM | 3133 | CA | TYR | D | 29 | −23.922 | 23.751 | 82.654 | 1.00 | 35.76 | D |
| ATOM | 3134 | CB | TYR | D | 29 | −23.690 | 22.542 | 83.558 | 1.00 | 34.09 | D |
| ATOM | 3135 | CG | TYR | D | 29 | −23.444 | 21.260 | 82.817 | 1.00 | 36.22 | D |
| ATOM | 3136 | CD1 | TYR | D | 29 | −22.404 | 21.152 | 81.893 | 1.00 | 36.27 | D |
| ATOM | 3137 | CE1 | TYR | D | 29 | −22.160 | 19.964 | 81.220 | 1.00 | 35.53 | D |
| ATOM | 3138 | CD2 | TYR | D | 29 | −24.239 | 20.143 | 83.047 | 1.00 | 39.14 | D |
| ATOM | 3139 | CE2 | TYR | D | 29 | −23.998 | 18.943 | 82.375 | 1.00 | 42.27 | D |
| ATOM | 3140 | CZ | TYR | D | 29 | −22.956 | 18.864 | 81.464 | 1.00 | 37.83 | D |
| ATOM | 3141 | OH | TYR | D | 29 | −22.716 | 17.682 | 80.810 | 1.00 | 38.69 | D |
| ATOM | 3142 | C | TYR | D | 29 | −25.066 | 23.487 | 81.678 | 1.00 | 34.54 | D |
| ATOM | 3143 | O | TYR | D | 29 | −26.226 | 23.443 | 82.082 | 1.00 | 36.75 | D |
| ATOM | 3144 | N | THR | D | 30 | −24.743 | 23.332 | 80.396 | 1.00 | 34.66 | D |
| ATOM | 3145 | CA | THR | D | 30 | −25.765 | 23.111 | 79.378 | 1.00 | 35.53 | D |
| ATOM | 3146 | CB | THR | D | 30 | −25.715 | 24.191 | 78.279 | 1.00 | 33.50 | D |
| ATOM | 3147 | OG1 | THR | D | 30 | −24.417 | 24.196 | 77.676 | 1.00 | 35.01 | D |
| ATOM | 3148 | CG2 | THR | D | 30 | −26.006 | 25.557 | 78.861 | 1.00 | 33.64 | D |
| ATOM | 3149 | C | THR | D | 30 | −25.666 | 21.769 | 78.691 | 1.00 | 36.80 | D |
| ATOM | 3150 | O | THR | D | 30 | −26.435 | 21.486 | 77.777 | 1.00 | 37.12 | D |
| ATOM | 3151 | N | GLY | D | 31 | −24.721 | 20.946 | 79.124 | 1.00 | 39.36 | D |
| ATOM | 3152 | CA | GLY | D | 31 | −24.563 | 19.640 | 78.517 | 1.00 | 41.27 | D |
| ATOM | 3153 | C | GLY | D | 31 | −25.659 | 18.685 | 78.942 | 1.00 | 42.13 | D |
| ATOM | 3154 | O | GLY | D | 31 | −26.139 | 18.755 | 80.069 | 1.00 | 40.15 | D |
| ATOM | 3155 | N | PHE | D | 32 | −26.048 | 17.794 | 78.034 | 1.00 | 44.77 | D |
| ATOM | 3156 | CA | PHE | D | 32 | −27.086 | 16.795 | 78.281 | 1.00 | 48.66 | D |
| ATOM | 3157 | CB | PHE | D | 32 | −26.571 | 15.731 | 79.250 | 1.00 | 47.57 | D |
| ATOM | 3158 | CG | PHE | D | 32 | −25.338 | 15.020 | 78.763 | 1.00 | 49.76 | D |
| ATOM | 3159 | CD1 | PHE | D | 32 | −24.078 | 15.591 | 78.924 | 1.00 | 46.08 | D |
| ATOM | 3160 | CD2 | PHE | D | 32 | −25.439 | 13.785 | 78.121 | 1.00 | 47.33 | D |
| ATOM | 3161 | CE1 | PHE | D | 32 | −22.942 | 14.944 | 78.456 | 1.00 | 44.69 | D |
| ATOM | 3162 | CE2 | PHE | D | 32 | −24.306 | 13.135 | 77.651 | 1.00 | 45.90 | D |
| ATOM | 3163 | CZ | PHE | D | 32 | −23.055 | 13.716 | 77.819 | 1.00 | 44.31 | D |
| ATOM | 3164 | C | PHE | D | 32 | −28.408 | 17.364 | 78.788 | 1.00 | 51.96 | D |
| ATOM | 3165 | O | PHE | D | 32 | −29.076 | 16.762 | 79.629 | 1.00 | 50.61 | D |
| ATOM | 3166 | N | ARG | D | 33 | −28.777 | 18.520 | 78.245 | 1.00 | 57.37 | D |
| ATOM | 3167 | CA | ARG | D | 33 | −30.007 | 19.222 | 78.596 | 1.00 | 63.26 | D |
| ATOM | 3168 | CB | ARG | D | 33 | −30.483 | 20.052 | 77.403 | 1.00 | 67.32 | D |
| ATOM | 3169 | CG | ARG | D | 33 | −30.967 | 21.450 | 77.750 | 1.00 | 72.99 | D |
| ATOM | 3170 | CD | ARG | D | 33 | −29.808 | 22.334 | 78.176 | 1.00 | 76.86 | D |
| ATOM | 3171 | NE | ARG | D | 33 | −30.254 | 23.672 | 78.564 | 1.00 | 82.38 | D |
| ATOM | 3172 | CZ | ARG | D | 33 | −30.052 | 24.213 | 79.766 | 1.00 | 84.06 | D |
| ATOM | 3173 | NH1 | ARG | D | 33 | −29.409 | 23.535 | 80.714 | 1.00 | 81.96 | D |
| ATOM | 3174 | NE2 | ARG | D | 33 | −30.490 | 25.439 | 80.020 | 1.00 | 85.14 | D |
| ATOM | 3175 | C | ARG | D | 33 | −31.114 | 18.261 | 79.012 | 1.00 | 65.68 | D |
| ATOM | 3176 | O | ARG | D | 33 | −31.539 | 18.251 | 80.165 | 1.00 | 66.69 | D |
| ATOM | 3177 | N | ASP | D | 34 | −31.587 | 17.458 | 78.067 | 1.00 | 68.88 | D |
| ATOM | 3178 | CA | ASP | D | 34 | −32.645 | 16.501 | 78.355 | 1.00 | 71.50 | D |
| ATOM | 3179 | CB | ASP | D | 34 | −33.488 | 16.232 | 77.109 | 1.00 | 75.94 | D |
| ATOM | 3180 | CG | ASP | D | 34 | −34.606 | 17.240 | 76.935 | 1.00 | 80.47 | D |
| ATOM | 3181 | OD1 | ASP | D | 34 | −34.309 | 18.449 | 76.817 | 1.00 | 83.24 | D |
| ATOM | 3182 | OD2 | ASP | D | 34 | −35.784 | 16.820 | 76.921 | 1.00 | 83.06 | D |
| ATOM | 3183 | C | ASP | D | 34 | −32.080 | 15.194 | 78.880 | 1.00 | 71.38 | D |
| ATOM | 3184 | O | ASP | D | 34 | −31.780 | 14.278 | 78.112 | 1.00 | 74.31 | D |
| ATOM | 3185 | N | ARG | D | 35 | −31.933 | 15.135 | 80.200 | 1.00 | 67.53 | D |
| ATOM | 3186 | CA | ARG | D | 35 | −31.420 | 13.972 | 80.903 | 1.00 | 62.80 | D |
| ATOM | 3187 | CB | ARG | D | 35 | −29.944 | 13.734 | 80.570 | 1.00 | 64.50 | D |
| ATOM | 3188 | CG | ARG | D | 35 | −29.725 | 12.735 | 79.447 | 1.00 | 65.78 | D |
| ATOM | 3189 | CD | ARG | D | 35 | −30.476 | 11.455 | 79.757 | 1.00 | 72.78 | D |
| ATOM | 3190 | NE | ARG | D | 35 | −30.404 | 10.465 | 78.690 | 1.00 | 77.33 | D |
| ATOM | 3191 | CZ | ARG | D | 35 | −31.115 | 9.340 | 78.677 | 1.00 | 79.91 | D |
| ATOM | 3192 | NH1 | ARG | D | 35 | −31.951 | 9.072 | 79.673 | 1.00 | 78.51 | D |
| ATOM | 3193 | NH2 | ARG | D | 35 | −30.986 | 8.480 | 77.673 | 1.00 | 81.41 | D |
| ATOM | 3194 | C | ARG | D | 35 | −31.581 | 14.216 | 82.393 | 1.00 | 61.05 | D |
| ATOM | 3195 | O | ARG | D | 35 | −31.364 | 15.328 | 82.872 | 1.00 | 62.46 | D |
| ATOM | 3196 | N | PRO | D | 36 | −31.986 | 13.181 | 83.145 | 1.00 | 58.03 | D |
| ATOM | 3197 | CD | PRO | D | 36 | −32.298 | 11.811 | 82.695 | 1.00 | 57.24 | D |
| ATOM | 3198 | CA | PRO | D | 36 | −32.172 | 13.306 | 84.590 | 1.00 | 54.18 | D |
| ATOM | 3199 | CB | PRO | D | 36 | −32.221 | 11.855 | 85.044 | 1.00 | 54.50 | D |
| ATOM | 3200 | CG | PRO | D | 36 | −32.966 | 11.209 | 83.916 | 1.00 | 55.03 | D |
| ATOM | 3201 | C | PRO | D | 36 | −31.051 | 14.104 | 85.250 | 1.00 | 52.16 | D |
| ATOM | 3202 | O | PRO | D | 36 | −29.881 | 13.937 | 84.921 | 1.00 | 50.28 | D |
| ATOM | 3203 | N | HIS | D | 37 | −31.432 | 14.973 | 86.180 | 1.00 | 50.22 | D |
| ATOM | 3204 | CA | HIS | D | 37 | −30.498 | 15.833 | 86.890 | 1.00 | 48.90 | D |
| ATOM | 3205 | CB | HIS | D | 37 | −31.239 | 16.634 | 87.963 | 1.00 | 47.70 | D |
| ATOM | 3206 | CG | HIS | D | 37 | −30.377 | 17.642 | 88.654 | 1.00 | 49.20 | D |
| ATOM | 3207 | CD2 | HIS | D | 37 | −29.834 | 17.650 | 89.895 | 1.00 | 47.87 | D |
| ATOM | 3208 | ND1 | HIS | D | 37 | −29.923 | 18.785 | 88.029 | 1.00 | 48.06 | D |
| ATOM | 3209 | CE1 | HIS | D | 37 | −29.137 | 19.452 | 88.854 | 1.00 | 47.70 | D |
| ATOM | 3210 | NE2 | HIS | D | 37 | −29.065 | 18.784 | 89.992 | 1.00 | 50.59 | D |
| ATOM | 3211 | C | HIS | D | 37 | −29.322 | 15.104 | 87.538 | 1.00 | 49.10 | D |
| ATOM | 3212 | O | HIS | D | 37 | −28.186 | 15.581 | 87.496 | 1.00 | 49.19 | D |
| ATOM | 3213 | N | GLU | D | 38 | −29.600 | 13.961 | 88.153 | 1.00 | 49.57 | D |
| ATOM | 3214 | CA | GLU | D | 38 | −28.563 | 13.179 | 88.813 | 1.00 | 50.12 | D |
| ATOM | 3215 | CB | GLU | D | 38 | −29.189 | 11.977 | 89.525 | 1.00 | 54.69 | D |
| ATOM | 3216 | CG | GLU | D | 38 | −30.132 | 12.344 | 90.694 | 1.00 | 65.17 | D |
| ATOM | 3217 | CD | GLU | D | 38 | −31.433 | 13.053 | 90.269 | 1.00 | 69.82 | D |
| ATOM | 3218 | OE1 | GLU | D | 38 | −32.214 | 12.476 | 89.473 | 1.00 | 71.51 | D |
| ATOM | 3219 | OE2 | GLU | D | 38 | −31.680 | 14.187 | 90.746 | 1.00 | 70.04 | D |
| ATOM | 3220 | C | GLU | D | 38 | −27.527 | 12.716 | 87.786 | 1.00 | 47.30 | D |
| ATOM | 3221 | O | GLU | D | 38 | −26.318 | 12.742 | 88.043 | 1.00 | 45.37 | D |
| ATOM | 3222 | N | GLU | D | 39 | −28.008 | 12.312 | 86.615 | 1.00 | 43.32 | D |
| ATOM | 3223 | CA | GLU | D | 39 | −27.126 | 11.866 | 85.555 | 1.00 | 41.99 | D |
| ATOM | 3224 | CB | GLU | D | 39 | −27.935 | 11.242 | 84.427 | 1.00 | 41.66 | D |
| ATOM | 3225 | CG | GLU | D | 39 | −27.067 | 10.563 | 83.396 | 1.00 | 44.89 | D |
| ATOM | 3226 | CD | GLU | D | 39 | −27.759 | 10.404 | 82.069 | 1.00 | 50.66 | D |
| ATOM | 3227 | OE1 | GLU | D | 39 | −28.911 | 9.908 | 82.047 | 1.00 | 52.32 | D |
| ATOM | 3228 | OE2 | GLU | D | 39 | −27.143 | 10.774 | 81.042 | 1.00 | 53.65 | D |
| ATOM | 3229 | C | GLU | D | 39 | −26.313 | 13.045 | 85.009 | 1.00 | 44.06 | D |
| ATOM | 3230 | O | GLU | D | 39 | −25.121 | 12.898 | 84.706 | 1.00 | 42.24 | D |
| ATOM | 3231 | N | ARG | D | 40 | −26.964 | 14.206 | 84.881 | 1.00 | 42.36 | D |
| ATOM | 3232 | CA | ARG | D | 40 | −26.309 | 15.411 | 84.383 | 1.00 | 42.48 | D |
| ATOM | 3233 | CB | ARG | D | 40 | −27.298 | 16.587 | 84.284 | 1.00 | 43.75 | D |
| ATOM | 3234 | CG | ARG | D | 40 | −28.410 | 16.392 | 83.247 | 1.00 | 42.33 | D |
| ATOM | 3235 | CD | ARG | D | 40 | −29.197 | 17.670 | 82.968 | 1.00 | 43.54 | D |
| ATOM | 3236 | NE | ARG | D | 40 | −28.453 | 18.618 | 82.136 | 1.00 | 46.23 | D |
| ATOM | 3237 | CZ | ARG | D | 40 | −28.054 | 19.832 | 82.520 | 1.00 | 45.47 | D |
| ATOM | 3238 | NH1 | ARG | D | 40 | −28.317 | 20.288 | 83.740 | 1.00 | 42.01 | D |
| ATOM | 3239 | NH2 | ARG | D | 40 | −27.383 | 20.597 | 81.673 | 1.00 | 41.40 | D |
| ATOM | 3240 | C | ARG | D | 40 | −25.175 | 15.768 | 85.323 | 1.00 | 42.34 | D |
| ATOM | 3241 | O | ARG | D | 40 | −24.109 | 16.211 | 84.889 | 1.00 | 41.62 | D |
| ATOM | 3242 | N | GLN | D | 41 | −25.408 | 15.557 | 86.613 | 1.00 | 41.78 | D |
| ATOM | 3243 | CA | GLN | D | 41 | −24.403 | 15.836 | 87.632 | 1.00 | 44.13 | D |
| ATOM | 3244 | CB | GLN | D | 41 | −24.985 | 15.579 | 89.034 | 1.00 | 46.96 | D |
| ATOM | 3245 | CG | GLN | D | 41 | −26.093 | 16.553 | 89.459 | 1.00 | 50.71 | D |
| ATOM | 3246 | CD | GLN | D | 41 | −26.738 | 16.183 | 90.792 | 1.00 | 54.69 | D |
| ATOM | 3247 | OE1 | GLN | D | 41 | −27.484 | 15.211 | 90.879 | 1.00 | 59.33 | D |
| ATOM | 3248 | NE2 | GLN | D | 41 | −26.447 | 16.956 | 91.835 | 1.00 | 54.13 | D |
| ATOM | 3249 | C | GLN | D | 41 | −23.175 | 14.955 | 87.405 | 1.00 | 44.32 | D |
| ATOM | 3250 | O | GLN | D | 41 | −22.039 | 15.403 | 87.549 | 1.00 | 45.90 | D |
| ATOM | 3251 | N | ALA | D | 42 | −23.409 | 13.696 | 87.045 | 1.00 | 46.20 | D |
| ATOM | 3252 | CA | ALA | D | 42 | −22.313 | 12.763 | 86.800 | 1.00 | 46.11 | D |
| ATOM | 3253 | CB | ALA | D | 42 | −22.855 | 11.346 | 86.636 | 1.00 | 44.93 | D |
| ATOM | 3254 | C | ALA | D | 42 | −21.551 | 13.187 | 85.549 | 1.00 | 44.54 | D |
| ATOM | 3255 | O | ALA | D | 42 | −20.334 | 13.370 | 85.590 | 1.00 | 43.31 | D |
| ATOM | 3256 | N | ARG | D | 43 | −22.284 | 13.336 | 84.447 | 1.00 | 42.94 | D |
| ATOM | 3257 | CA | ARG | D | 43 | −21.720 | 13.762 | 83.165 | 1.00 | 43.87 | D |
| ATOM | 3258 | CB | ARG | D | 43 | −22.831 | 14.163 | 82.190 | 1.00 | 46.07 | D |
| ATOM | 3259 | CG | ARG | D | 43 | −23.876 | 13.109 | 81.917 | 1.00 | 45.43 | D |
| ATOM | 3260 | CD | ARG | D | 43 | −23.341 | 12.080 | 80.982 | 1.00 | 45.32 | D |
| ATOM | 3261 | NE | ARG | D | 43 | −24.336 | 11.069 | 80.670 | 1.00 | 43.69 | D |
| ATOM | 3262 | CZ | ARG | D | 43 | −24.102 | 10.051 | 79.860 | 1.00 | 39.05 | D |
| ATOM | 3263 | NH1 | ARG | D | 43 | −22.915 | 9.933 | 79.293 | 1.00 | 37.89 | D |
| ATOM | 3264 | NH2 | ARG | D | 43 | −25.041 | 9.151 | 79.633 | 1.00 | 41.52 | D |
| ATOM | 3265 | C | ARG | D | 43 | −20.818 | 14.975 | 83.352 | 1.00 | 44.48 | D |
| ATOM | 3266 | O | ARG | D | 43 | −19.675 | 14.983 | 82.889 | 1.00 | 45.13 | D |
| ATOM | 3267 | N | PHE | D | 44 | −21.351 | 15.998 | 84.026 | 1.00 | 42.72 | D |
| ATOM | 3268 | CA | PHE | D | 44 | −20.618 | 17.236 | 84.269 | 1.00 | 40.60 | D |
| ATOM | 3269 | CB | PHE | D | 44 | −21.437 | 18.193 | 85.134 | 1.00 | 37.96 | D |
| ATOM | 3270 | CG | PHE | D | 44 | −20.712 | 19.466 | 85.465 | 1.00 | 35.11 | D |
| ATOM | 3271 | CD1 | PHE | D | 44 | −20.403 | 20.382 | 84.465 | 1.00 | 32.05 | D |
| ATOM | 3272 | CD2 | PHE | D | 44 | −20.287 | 19.724 | 86.769 | 1.00 | 33.36 | D |
| ATOM | 3273 | CE1 | PHE | D | 44 | −19.682 | 21.526 | 84.752 | 1.00 | 29.81 | D |
| ATOM | 3274 | CE2 | PHE | D | 44 | −19.565 | 20.866 | 87.069 | 1.00 | 30.30 | D |
| ATOM | 3275 | CZ | PHE | D | 44 | −19.260 | 21.771 | 86.057 | 1.00 | 33.23 | D |
| ATOM | 3276 | C | PHE | D | 44 | −19.267 | 17.002 | 84.922 | 1.00 | 42.52 | D |
| ATOM | 3277 | O | PHE | D | 44 | −18.258 | 17.578 | 84.501 | 1.00 | 42.86 | D |
| ATOM | 3278 | N | GLN | D | 45 | −19.241 | 16.168 | 85.956 | 1.00 | 45.73 | D |
| ATOM | 3279 | CA | GLN | D | 45 | −17.983 | 15.875 | 86.624 | 1.00 | 50.22 | D |
| ATOM | 3280 | CB | GLN | D | 45 | −18.229 | 15.131 | 87.924 | 1.00 | 54.78 | D |
| ATOM | 3281 | CG | GLN | D | 45 | −18.067 | 16.003 | 89.133 | 1.00 | 60.43 | D |
| ATOM | 3282 | CD | GLN | D | 45 | −17.527 | 15.229 | 90.301 | 1.00 | 64.60 | D |
| ATOM | 3283 | OE1 | GLN | D | 45 | −16.455 | 14.610 | 90.210 | 1.00 | 66.99 | D |
| ATOM | 3284 | NE2 | GLN | D | 45 | −18.258 | 15.251 | 91.413 | 1.00 | 63.21 | D |
| ATOM | 3285 | C | GLN | D | 45 | −17.044 | 15.059 | 85.738 | 1.00 | 51.81 | D |
| ATOM | 3286 | O | GLN | D | 45 | −15.834 | 15.294 | 85.727 | 1.00 | 52.56 | D |
| ATOM | 3287 | N | ASN | D | 46 | −17.598 | 14.100 | 85.002 | 1.00 | 49.81 | D |
| ATOM | 3288 | CA | ASN | D | 46 | −16.790 | 13.284 | 84.107 | 1.00 | 52.31 | D |
| ATOM | 3289 | CB | ASN | D | 46 | −17.646 | 12.255 | 83.360 | 1.00 | 57.21 | D |
| ATOM | 3290 | CG | ASN | D | 46 | −17.802 | 10.956 | 84.124 | 1.00 | 60.48 | D |
| ATOM | 3291 | OD1 | ASN | D | 46 | −17.062 | 10.687 | 85.079 | 1.00 | 61.70 | D |
| ATOM | 3292 | ND2 | ASN | D | 46 | −18.756 | 10.130 | 83.696 | 1.00 | 58.80 | D |
| ATOM | 3293 | C | ASN | D | 46 | −16.103 | 14.173 | 83.089 | 1.00 | 52.01 | D |
| ATOM | 3294 | O | ASN | D | 46 | −14.882 | 14.137 | 82.956 | 1.00 | 53.21 | D |
| ATOM | 3295 | N | ALA | D | 47 | −16.897 | 14.968 | 82.374 | 1.00 | 49.56 | D |
| ATOM | 3296 | CA | ALA | D | 47 | −16.380 | 15.878 | 81.353 | 1.00 | 45.44 | D |
| ATOM | 3297 | CB | ALA | D | 47 | −17.536 | 16.629 | 80.694 | 1.00 | 44.88 | D |
| ATOM | 3298 | C | ALA | D | 47 | −15.364 | 16.871 | 81.925 | 1.00 | 43.33 | D |
| ATOM | 3299 | O | ALA | D | 47 | −14.358 | 17.169 | 81.285 | 1.00 | 36.48 | D |
| ATOM | 3300 | N | CYS | D | 48 | −15.638 | 17.375 | 83.128 | 1.00 | 43.95 | D |
| ATOM | 3301 | CA | CYS | D | 48 | −14.749 | 18.327 | 83.792 | 1.00 | 48.74 | D |
| ATOM | 3302 | CB | CYS | D | 48 | −15.347 | 18.798 | 85.112 | 1.00 | 49.72 | D |
| ATOM | 3303 | SG | CYS | D | 48 | −16.425 | 20.209 | 84.999 | 1.00 | 58.34 | D |
| ATOM | 3304 | C | CYS | D | 48 | −13.389 | 17.736 | 84.094 | 1.00 | 50.05 | D |
| ATOM | 3305 | O | CYS | D | 48 | −12.360 | 18.288 | 83.714 | 1.00 | 47.29 | D |
| ATOM | 3306 | N | ARG | D | 49 | −13.404 | 16.613 | 84.807 | 1.00 | 55.29 | D |
| ATOM | 3307 | CA | ARG | D | 49 | −12.183 | 15.934 | 85.206 | 1.00 | 58.19 | D |
| ATOM | 3308 | CB | ARG | D | 49 | −12.516 | 14.703 | 86.063 | 1.00 | 61.81 | D |
| ATOM | 3309 | CG | ARG | D | 49 | −13.109 | 15.078 | 87.423 | 1.00 | 67.72 | D |
| ATOM | 3310 | CD | ARG | D | 49 | −13.218 | 13.906 | 88.399 | 1.00 | 73.74 | D |
| ATOM | 3311 | NE | ARG | D | 49 | −14.146 | 12.873 | 87.946 | 1.00 | 79.14 | D |
| ATOM | 3312 | CZ | ARG | D | 49 | −13.795 | 11.825 | 87.206 | 1.00 | 81.93 | D |
| ATOM | 3313 | NH1 | ARG | D | 49 | −12.531 | 11.666 | 86.838 | 1.00 | 81.86 | D |
| ATOM | 3314 | NH2 | ARG | D | 49 | −14.709 | 10.939 | 86.828 | 1.00 | 82.60 | D |
| ATOM | 3315 | C | ARG | D | 49 | −11.360 | 15.555 | 83.992 | 1.00 | 57.76 | D |
| ATOM | 3316 | O | ARG | D | 49 | −10.144 | 15.390 | 84.087 | 1.00 | 58.58 | D |
| ATOM | 3317 | N | ASP | D | 50 | −12.029 | 15.429 | 82.850 | 1.00 | 56.34 | D |
| ATOM | 3318 | CA | ASP | D | 50 | −11.351 | 15.095 | 81.607 | 1.00 | 54.87 | D |
| ATOM | 3319 | CB | ASP | D | 50 | −12.175 | 14.074 | 80.814 | 1.00 | 56.86 | D |
| ATOM | 3320 | CG | ASP | D | 50 | −12.220 | 12.701 | 81.505 | 1.00 | 62.27 | D |
| ATOM | 3321 | OD1 | ASP | D | 50 | −11.139 | 12.155 | 81.823 | 1.00 | 61.09 | D |
| ATOM | 3322 | OD2 | ASP | D | 50 | −13.329 | 12.164 | 81.731 | 1.00 | 62.30 | D |
| ATOM | 3323 | C | ASP | D | 50 | −11.055 | 16.358 | 80.787 | 1.00 | 52.47 | D |
| ATOM | 3324 | O | ASP | D | 50 | −10.765 | 16.287 | 79.592 | 1.00 | 52.50 | D |
| ATOM | 3325 | N | GLY | D | 51 | −11.133 | 17.507 | 81.465 | 1.00 | 48.86 | D |
| ATOM | 3326 | CA | GLY | D | 51 | −10.827 | 18.800 | 80.874 | 1.00 | 46.49 | D |
| ATOM | 3327 | C | GLY | D | 51 | −11.814 | 19.561 | 79.998 | 1.00 | 46.07 | D |
| ATOM | 3328 | O | GLY | D | 51 | −11.436 | 20.580 | 79.417 | 1.00 | 43.75 | D |
| ATOM | 3329 | N | ARG | D | 52 | −13.064 | 19.121 | 79.900 | 1.00 | 46.32 | D |
| ATOM | 3330 | CA | ARG | D | 52 | −14.009 | 19.827 | 79.040 | 1.00 | 47.23 | D |
| ATOM | 3331 | CB | ARG | D | 52 | −14.157 | 19.092 | 77.700 | 1.00 | 47.29 | D |
| ATOM | 3332 | CG | ARG | D | 52 | −14.747 | 17.693 | 77.821 | 1.00 | 52.66 | D |
| ATOM | 3333 | CD | ARG | D | 52 | −15.181 | 17.073 | 76.463 | 1.00 | 58.80 | D |
| ATOM | 3334 | NE | ARG | D | 52 | −16.603 | 17.299 | 76.158 | 1.00 | 64.74 | D |
| ATOM | 3335 | CZ | ARG | D | 52 | −17.136 | 18.489 | 75.852 | 1.00 | 68.30 | D |
| ATOM | 3336 | NH1 | ARG | D | 52 | −16.370 | 19.581 | 75.795 | 1.00 | 67.57 | D |
| ATOM | 3337 | NH2 | ARG | D | 52 | −18.446 | 18.599 | 75.619 | 1.00 | 69.20 | D |
| ATOM | 3338 | C | ARG | D | 52 | −15.393 | 20.038 | 79.631 | 1.00 | 45.70 | D |
| ATOM | 3339 | O | ARG | D | 52 | −15.777 | 19.384 | 80.598 | 1.00 | 46.10 | D |
| ATOM | 3340 | N | SER | D | 53 | −16.136 | 20.969 | 79.035 | 1.00 | 43.63 | D |
| ATOM | 3341 | CA | SER | D | 53 | −17.498 | 21.261 | 79.455 | 1.00 | 42.85 | D |
| ATOM | 3342 | CB | SER | D | 53 | −17.517 | 21.839 | 80.871 | 1.00 | 42.86 | D |
| ATOM | 3343 | OG | SER | D | 53 | −18.843 | 21.847 | 81.380 | 1.00 | 41.40 | D |
| ATOM | 3344 | C | SER | D | 53 | −18.234 | 22.212 | 78.511 | 1.00 | 42.00 | D |
| ATOM | 3345 | O | SER | D | 53 | −17.646 | 22.809 | 77.613 | 1.00 | 39.88 | D |
| ATOM | 3346 | N | GLU | D | 54 | −19.540 | 22.317 | 78.720 | 1.00 | 41.99 | D |
| ATOM | 3347 | CA | GLU | D | 54 | −20.398 | 23.196 | 77.944 | 1.00 | 41.43 | D |
| ATOM | 3348 | CB | GLU | D | 54 | −21.403 | 22.391 | 77.123 | 1.00 | 46.74 | D |
| ATOM | 3349 | CG | GLU | D | 54 | −20.905 | 21.996 | 75.742 | 1.00 | 58.42 | D |
| ATOM | 3350 | CD | GLU | D | 54 | −21.902 | 21.131 | 74.984 | 1.00 | 65.40 | D |
| ATOM | 3351 | OE1 | GLU | D | 54 | −23.109 | 21.475 | 74.969 | 1.00 | 66.89 | D |
| ATOM | 3352 | OE2 | GLU | D | 54 | −21.473 | 20.113 | 74.398 | 1.00 | 67.41 | D |
| ATOM | 3353 | C | GLU | D | 54 | −21.119 | 24.049 | 78.971 | 1.00 | 40.46 | D |
| ATOM | 3354 | O | GLU | D | 54 | −21.911 | 23.553 | 79.772 | 1.00 | 36.62 | D |
| ATOM | 3355 | N | ILE | D | 55 | −20.829 | 25.339 | 78.958 | 1.00 | 39.43 | D |
| ATOM | 3356 | CA | ILE | D | 55 | −21.429 | 26.240 | 79.924 | 1.00 | 40.63 | D |
| ATOM | 3357 | CB | ILE | D | 55 | −20.405 | 26.579 | 81.043 | 1.00 | 38.86 | D |
| ATOM | 3358 | CG2 | ILE | D | 55 | −21.000 | 27.546 | 82.037 | 1.00 | 40.38 | D |
| ATOM | 3359 | CG1 | ILE | D | 55 | −19.999 | 25.292 | 81.763 | 1.00 | 39.37 | D |
| ATOM | 3360 | CD1 | ILE | D | 55 | −18.785 | 25.422 | 82.659 | 1.00 | 43.37 | D |
| ATOM | 3361 | C | ILE | D | 55 | −21.890 | 27.497 | 79.221 | 1.00 | 39.71 | D |
| ATOM | 3362 | O | ILE | D | 55 | −21.217 | 28.006 | 78.331 | 1.00 | 42.75 | D |
| ATOM | 3363 | N | ALA | D | 56 | −23.054 | 27.987 | 79.609 | 1.00 | 39.61 | D |
| ATOM | 3364 | CA | ALA | D | 56 | −23.590 | 29.188 | 79.003 | 1.00 | 40.47 | D |
| ATOM | 3365 | CB | ALA | D | 56 | −24.898 | 28.874 | 78.313 | 1.00 | 36.69 | D |
| ATOM | 3366 | C | ALA | D | 56 | −23.812 | 30.271 | 80.047 | 1.00 | 43.61 | D |
| ATOM | 3367 | O | ALA | D | 56 | −24.014 | 29.987 | 81.235 | 1.00 | 41.32 | D |
| ATOM | 3368 | N | PHE | D | 57 | −23.730 | 31.522 | 79.609 | 1.00 | 44.78 | D |
| ATOM | 3369 | CA | PHE | D | 57 | −24.018 | 32.629 | 80.505 | 1.00 | 44.18 | D |
| ATOM | 3370 | CB | PHE | D | 57 | −23.311 | 33.904 | 80.045 | 1.00 | 41.07 | D |
| ATOM | 3371 | CG | PHE | D | 57 | −21.816 | 33.862 | 80.198 | 1.00 | 37.32 | D |
| ATOM | 3372 | CD1 | PHE | D | 57 | −21.005 | 33.455 | 79.145 | 1.00 | 38.16 | D |
| ATOM | 3373 | CD2 | PHE | D | 57 | −21.220 | 34.245 | 81.389 | 1.00 | 35.21 | D |
| ATOM | 3374 | CE1 | PHE | D | 57 | −19.615 | 33.433 | 79.272 | 1.00 | 38.22 | D |
| ATOM | 3375 | CE2 | PHE | D | 57 | −19.835 | 34.230 | 81.536 | 1.00 | 37.12 | D |
| ATOM | 3376 | CZ | PHE | D | 57 | −19.026 | 33.821 | 80.469 | 1.00 | 40.01 | D |
| ATOM | 3377 | C | PHE | D | 57 | −25.541 | 32.741 | 80.332 | 1.00 | 45.18 | D |
| ATOM | 3378 | O | PHE | D | 57 | −26.028 | 33.005 | 79.226 | 1.00 | 43.40 | D |
| ATOM | 3379 | N | VAL | D | 58 | −26.286 | 32.511 | 81.411 | 1.00 | 45.43 | D |
| ATOM | 3380 | CA | VAL | D | 58 | −27.749 | 32.531 | 81.337 | 1.00 | 48.05 | D |
| ATOM | 3381 | CB | VAL | D | 58 | −28.388 | 32.053 | 82.659 | 1.00 | 47.27 | D |
| ATOM | 3382 | CG1 | VAL | D | 58 | −27.901 | 30.653 | 82.976 | 1.00 | 51.12 | D |
| ATOM | 3383 | CG2 | VAL | D | 58 | −28.051 | 33.004 | 83.792 | 1.00 | 48.21 | D |
| ATOM | 3384 | C | VAL | D | 58 | −28.403 | 33.849 | 80.941 | 1.00 | 48.22 | D |
| ATOM | 3385 | O | VAL | D | 58 | −29.264 | 33.871 | 80.063 | 1.00 | 47.14 | D |
| ATOM | 3386 | N | ALA | D | 59 | −28.002 | 34.942 | 81.581 | 1.00 | 47.51 | D |
| ATOM | 3387 | CA | ALA | D | 59 | −28.587 | 36.237 | 81.275 | 1.00 | 49.66 | D |
| ATOM | 3388 | CB | ALA | D | 59 | −27.870 | 37.342 | 82.058 | 1.00 | 47.26 | D |
| ATOM | 3389 | C | ALA | D | 59 | −28.527 | 36.520 | 79.780 | 1.00 | 49.57 | D |
| ATOM | 3390 | O | ALA | D | 59 | −29.554 | 36.638 | 79.124 | 1.00 | 52.91 | D |
| ATOM | 3391 | N | THR | D | 60 | −27.322 | 36.611 | 79.240 | 1.00 | 49.87 | D |
| ATOM | 3392 | CA | THR | D | 60 | −27.150 | 36.896 | 77.820 | 1.00 | 51.27 | D |
| ATOM | 3393 | CB | THR | D | 60 | −25.734 | 37.404 | 77.549 | 1.00 | 49.16 | D |
| ATOM | 3394 | OG1 | THR | D | 60 | −24.803 | 36.550 | 78.218 | 1.00 | 49.93 | D |
| ATOM | 3395 | CG2 | THR | D | 60 | −25.567 | 38.837 | 78.054 | 1.00 | 49.54 | D |
| ATOM | 3396 | C | THR | D | 60 | −27.431 | 35.720 | 76.884 | 1.00 | 50.78 | D |
| ATOM | 3397 | O | THR | D | 60 | −27.708 | 35.919 | 75.708 | 1.00 | 49.94 | D |
| ATOM | 3398 | N | GLY | D | 61 | −27.351 | 34.498 | 77.403 | 1.00 | 52.07 | D |
| ATOM | 3399 | CA | GLY | D | 61 | −27.597 | 33.329 | 76.573 | 1.00 | 53.13 | D |
| ATOM | 3400 | C | GLY | D | 61 | −26.398 | 32.925 | 75.725 | 1.00 | 52.59 | D |
| ATOM | 3401 | O | GLY | D | 61 | −26.555 | 32.321 | 74.664 | 1.00 | 55.62 | D |
| ATOM | 3402 | N | THR | D | 62 | −25.200 | 33.253 | 76.202 | 1.00 | 50.41 | D |
| ATOM | 3403 | CA | THR | D | 62 | −23.961 | 32.938 | 75.498 | 1.00 | 46.31 | D |
| ATOM | 3404 | CB | THR | D | 62 | −22.872 | 34.002 | 75.801 | 1.00 | 48.09 | D |
| ATOM | 3405 | OG1 | THR | D | 62 | −23.381 | 35.305 | 75.486 | 1.00 | 51.58 | D |
| ATOM | 3406 | CG2 | THR | D | 62 | −21.618 | 33.753 | 74.970 | 1.00 | 46.02 | D |
| ATOM | 3407 | C | THR | D | 62 | −23.481 | 31.560 | 75.949 | 1.00 | 43.88 | D |
| ATOM | 3408 | O | THR | D | 62 | −23.051 | 31.379 | 77.090 | 1.00 | 44.53 | D |
| ATOM | 3409 | N | ASN | D | 63 | −23.559 | 30.592 | 75.042 | 1.00 | 38.87 | D |
| ATOM | 3410 | CA | ASN | D | 63 | −23.169 | 29.226 | 75.337 | 1.00 | 36.43 | D |
| ATOM | 3411 | CB | ASN | D | 63 | −24.226 | 28.287 | 74.749 | 1.00 | 39.06 | D |
| ATOM | 3412 | CG | ASN | D | 63 | −24.031 | 26.847 | 75.159 | 1.00 | 43.42 | D |
| ATOM | 3413 | OD1 | ASN | D | 63 | −23.734 | 26.545 | 76.319 | 1.00 | 43.05 | D |
| ATOM | 3414 | ND2 | ASN | D | 63 | −24.218 | 25.942 | 74.212 | 1.00 | 44.50 | D |
| ATOM | 3415 | C | ASN | D | 63 | −21.775 | 28.922 | 74.780 | 1.00 | 36.20 | D |
| ATOM | 3416 | O | ASN | D | 63 | −21.479 | 29.240 | 73.634 | 1.00 | 35.27 | D |
| ATOM | 3417 | N | LEU | D | 64 | −20.922 | 28.302 | 75.591 | 1.00 | 34.33 | D |
| ATOM | 3418 | CA | LEU | D | 64 | −19.563 | 27.998 | 75.166 | 1.00 | 33.55 | D |
| ATOM | 3419 | CB | LEU | D | 64 | −18.602 | 28.999 | 75.804 | 1.00 | 34.26 | D |
| ATOM | 3420 | CG | LEU | D | 64 | −18.782 | 30.453 | 75.363 | 1.00 | 35.21 | D |
| ATOM | 3421 | CD1 | LEU | D | 64 | −18.052 | 31.375 | 76.330 | 1.00 | 39.96 | D |
| ATOM | 3422 | CD2 | LEU | D | 64 | −18.254 | 30.622 | 73.934 | 1.00 | 35.32 | D |
| ATOM | 3423 | C | LEU | D | 64 | −19.073 | 26.583 | 75.460 | 1.00 | 33.59 | D |
| ATOM | 3424 | O | LEU | D | 64 | −19.430 | 25.981 | 76.475 | 1.00 | 31.60 | D |
| ATOM | 3425 | N | SER | D | 65 | −18.257 | 26.061 | 74.545 | 1.00 | 33.13 | D |
| ATOM | 3426 | CA | SER | D | 65 | −17.656 | 24.740 | 74.684 | 1.00 | 32.27 | D |
| ATOM | 3427 | CB | SER | D | 65 | −17.521 | 24.058 | 73.321 | 1.00 | 32.92 | D |
| ATOM | 3428 | OG | SER | D | 65 | −16.918 | 22.776 | 73.435 | 1.00 | 36.38 | D |
| ATOM | 3429 | C | SER | D | 65 | −16.286 | 25.079 | 75.256 | 1.00 | 32.62 | D |
| ATOM | 3430 | O | SER | D | 65 | −15.546 | 25.874 | 74.675 | 1.00 | 31.39 | D |
| ATOM | 3431 | N | LEU | D | 66 | −15.943 | 24.479 | 76.390 | 1.00 | 32.39 | D |
| ATOM | 3432 | CA | LEU | D | 66 | −14.691 | 24.813 | 77.050 | 1.00 | 34.93 | D |
| ATOM | 3433 | CB | LEU | D | 66 | −15.004 | 25.409 | 78.426 | 1.00 | 32.13 | D |
| ATOM | 3434 | CG | LEU | D | 66 | −16.005 | 26.565 | 78.418 | 1.00 | 34.79 | D |
| ATOM | 3435 | CD1 | LEU | D | 66 | −16.437 | 26.913 | 79.839 | 1.00 | 35.01 | D |
| ATOM | 3436 | CD2 | LEU | D | 66 | −15.373 | 27.753 | 77.731 | 1.00 | 35.61 | D |
| ATOM | 3437 | C | LEU | D | 66 | −13.664 | 23.709 | 77.224 | 1.00 | 36.40 | D |
| ATOM | 3438 | O | LEU | D | 66 | −14.006 | 22.561 | 77.520 | 1.00 | 35.68 | D |
| ATOM | 3439 | N | GLN | D | 67 | −12.400 | 24.079 | 77.041 | 1.00 | 35.91 | D |
| ATOM | 3440 | CA | GLN | D | 67 | −11.283 | 23.159 | 77.235 | 1.00 | 41.01 | D |
| ATOM | 3441 | CB | GLN | D | 67 | −10.443 | 23.008 | 75.964 | 1.00 | 40.55 | D |
| ATOM | 3442 | CG | GLN | D | 67 | −11.191 | 22.325 | 74.845 | 1.00 | 49.45 | D |
| ATOM | 3443 | CD | GLN | D | 67 | −10.278 | 21.648 | 73.854 | 1.00 | 51.92 | D |
| ATOM | 3444 | OE1 | GLN | D | 67 | −9.358 | 22.264 | 73.312 | 1.00 | 56.25 | D |
| ATOM | 3445 | NE2 | GLN | D | 67 | −10.529 | 20.369 | 73.605 | 1.00 | 52.99 | D |
| ATOM | 3446 | C | GLN | D | 67 | −10.433 | 23.732 | 78.362 | 1.00 | 40.47 | D |
| ATOM | 3447 | O | GLN | D | 67 | −9.839 | 24.805 | 78.226 | 1.00 | 41.43 | D |
| ATOM | 3448 | N | PHE | D | 68 | −10.396 | 23.016 | 79.480 | 1.00 | 41.08 | D |
| ATOM | 3449 | CA | PHE | D | 68 | −9.644 | 23.449 | 80.649 | 1.00 | 43.88 | D |
| ATOM | 3450 | CB | PHE | D | 68 | −10.313 | 22.883 | 81.909 | 1.00 | 42.14 | D |
| ATOM | 3451 | CG | PHE | D | 68 | −11.809 | 23.095 | 81.948 | 1.00 | 39.65 | D |
| ATOM | 3452 | CD1 | PHE | D | 68 | −12.682 | 22.011 | 81.901 | 1.00 | 38.99 | D |
| ATOM | 3453 | CD2 | PHE | D | 68 | −12.343 | 24.380 | 82.006 | 1.00 | 36.16 | D |
| ATOM | 3454 | CE1 | PHE | D | 68 | −14.061 | 22.203 | 81.912 | 1.00 | 36.72 | D |
| ATOM | 3455 | CE2 | PHE | D | 68 | −13.718 | 24.584 | 82.016 | 1.00 | 35.22 | D |
| ATOM | 3456 | CZ | PHE | D | 68 | −14.580 | 23.498 | 81.970 | 1.00 | 34.57 | D |
| ATOM | 3457 | C | PHE | D | 68 | −8.178 | 23.010 | 80.578 | 1.00 | 46.10 | D |
| ATOM | 3458 | O | PHE | D | 68 | −7.786 | 22.039 | 81.217 | 1.00 | 48.32 | D |
| ATOM | 3459 | N | PHE | D | 69 | −7.378 | 23.725 | 79.791 | 1.00 | 48.75 | D |
| ATOM | 3460 | CA | PHE | D | 69 | −5.952 | 23.423 | 79.631 | 1.00 | 52.27 | D |
| ATOM | 3461 | CB | PHE | D | 69 | −5.722 | 22.409 | 78.514 | 1.00 | 53.41 | D |
| ATOM | 3462 | CG | PHE | D | 69 | −6.385 | 21.093 | 78.736 | 1.00 | 54.68 | D |
| ATOM | 3463 | CD1 | PHE | D | 69 | −7.583 | 20.792 | 78.098 | 1.00 | 54.09 | D |
| ATOM | 3464 | CD2 | PHE | D | 69 | −5.802 | 20.138 | 79.572 | 1.00 | 56.24 | D |
| ATOM | 3465 | CE1 | PHE | D | 69 | −8.197 | 19.551 | 78.284 | 1.00 | 56.97 | D |
| ATOM | 3466 | CE2 | PHE | D | 69 | −6.406 | 18.889 | 79.770 | 1.00 | 56.59 | D |
| ATOM | 3467 | CZ | PHE | D | 69 | −7.606 | 18.595 | 79.124 | 1.00 | 57.27 | D |
| ATOM | 3468 | C | PHE | D | 69 | −5.205 | 24.698 | 79.264 | 1.00 | 54.75 | D |
| ATOM | 3469 | O | PHE | D | 69 | −5.804 | 25.637 | 78.733 | 1.00 | 58.60 | D |
| ATOM | 3470 | N | PRO | D | 70 | −3.886 | 24.747 | 79.522 | 1.00 | 55.35 | D |
| ATOM | 3471 | CD | PRO | D | 70 | −3.035 | 23.705 | 80.122 | 1.00 | 56.70 | D |
| ATOM | 3472 | CA | PRO | D | 70 | −3.093 | 25.942 | 79.198 | 1.00 | 55.34 | D |
| ATOM | 3473 | CB | PRO | D | 70 | −1.666 | 25.509 | 79.520 | 1.00 | 52.10 | D |
| ATOM | 3474 | CG | PRO | D | 70 | −1.859 | 24.507 | 80.614 | 1.00 | 55.34 | D |
| ATOM | 3475 | C | PRO | D | 70 | −3.254 | 26.339 | 77.728 | 1.00 | 55.13 | D |
| ATOM | 3476 | O | PRO | D | 70 | −3.286 | 25.470 | 76.848 | 1.00 | 57.18 | D |
| ATOM | 3477 | N | THR | D | 80 | −5.284 | 14.542 | 81.876 | 1.00 | 88.24 | D |
| ATOM | 3478 | CA | THR | D | 80 | −5.887 | 15.058 | 83.101 | 1.00 | 88.46 | D |
| ATOM | 3479 | CB | THR | D | 80 | −5.531 | 14.175 | 84.323 | 1.00 | 87.92 | D |
| ATOM | 3480 | OG1 | THR | D | 80 | −6.281 | 12.958 | 84.255 | 1.00 | 88.80 | D |
| ATOM | 3481 | CG2 | THR | D | 80 | −5.861 | 14.891 | 85.634 | 1.00 | 88.08 | D |
| ATOM | 3482 | C | THR | D | 80 | −5.465 | 16.493 | 83.395 | 1.00 | 87.62 | D |
| ATOM | 3483 | O | THR | D | 80 | −4.320 | 16.756 | 83.773 | 1.00 | 88.15 | D |
| ATOM | 3484 | N | PRO | D | 81 | −6.400 | 17.442 | 83.238 | 1.00 | 85.95 | D |
| ATOM | 3485 | CD | PRO | D | 81 | −7.843 | 17.245 | 83.002 | 1.00 | 84.54 | D |
| ATOM | 3486 | CA | PRO | D | 81 | −6.097 | 18.853 | 83.496 | 1.00 | 84.00 | D |
| ATOM | 3487 | CB | PRO | D | 81 | −7.442 | 19.541 | 83.250 | 1.00 | 84.44 | D |
| ATOM | 3488 | CG | PRO | D | 81 | −8.439 | 18.471 | 83.646 | 1.00 | 84.23 | D |
| ATOM | 3489 | C | PRO | D | 81 | −5.592 | 19.043 | 84.923 | 1.00 | 80.98 | D |
| ATOM | 3490 | O | PRO | D | 81 | −6.024 | 18.337 | 85.832 | 1.00 | 79.31 | D |
| ATOM | 3491 | N | SER | D | 82 | −4.681 | 19.989 | 85.120 | 1.00 | 78.98 | D |
| ATOM | 3492 | CA | SER | D | 82 | −4.156 | 20.235 | 86.455 | 1.00 | 78.40 | D |
| ATOM | 3493 | CB | SER | D | 82 | −3.011 | 21.252 | 86.416 | 1.00 | 78.27 | D |
| ATOM | 3494 | OG | SER | D | 82 | −3.502 | 22.571 | 86.257 | 1.00 | 76.48 | D |
| ATOM | 3495 | C | SER | D | 82 | −5.268 | 20.759 | 87.356 | 1.00 | 78.34 | D |
| ATOM | 3496 | O | SER | D | 82 | −6.448 | 20.707 | 86.997 | 1.00 | 77.46 | D |
| ATOM | 3497 | N | ARG | D | 83 | −4.876 | 21.260 | 88.527 | 1.00 | 78.62 | D |
| ATOM | 3498 | CA | ARG | D | 83 | −5.813 | 21.805 | 89.507 | 1.00 | 77.61 | D |
| ATOM | 3499 | CB | ARG | D | 83 | −5.262 | 21.616 | 90.923 | 0.00 | 77.60 | D |
| ATOM | 3500 | CG | ARG | D | 83 | −4.940 | 20.174 | 91.278 | 0.00 | 77.43 | D |
| ATOM | 3501 | CD | ARG | D | 83 | −4.323 | 20.073 | 92.664 | 0.00 | 77.33 | D |
| ATOM | 3502 | NE | ARG | D | 83 | −3.981 | 18.698 | 93.014 | 0.00 | 77.27 | D |
| ATOM | 3503 | CZ | ARG | D | 83 | −3.421 | 18.336 | 94.164 | 0.00 | 77.22 | D |
| ATOM | 3504 | NH1 | ARG | D | 83 | −3.136 | 19.249 | 95.082 | 0.00 | 77.21 | D |
| ATOM | 3505 | NH2 | ARG | D | 83 | −3.145 | 17.060 | 94.397 | 0.00 | 77.22 | D |
| ATOM | 3506 | C | ARG | D | 83 | −6.012 | 23.292 | 89.230 | 1.00 | 76.94 | D |
| ATOM | 3507 | O | ARG | D | 83 | −7.022 | 23.885 | 89.618 | 1.00 | 76.72 | D |
| ATOM | 3508 | N | GLU | D | 84 | −5.034 | 23.888 | 88.559 | 1.00 | 75.74 | D |
| ATOM | 3509 | CA | GLU | D | 84 | −5.099 | 25.298 | 88.218 | 1.00 | 76.59 | D |
| ATOM | 3510 | CB | GLU | D | 84 | −3.823 | 25.724 | 87.488 | 1.00 | 77.36 | D |
| ATOM | 3511 | CG | GLU | D | 84 | −3.745 | 27.212 | 87.186 | 1.00 | 81.57 | D |
| ATOM | 3512 | CD | GLU | D | 84 | −2.542 | 27.572 | 86.323 | 1.00 | 85.53 | D |
| ATOM | 3513 | OE1 | GLU | D | 84 | −1.449 | 27.007 | 86.560 | 1.00 | 87.11 | D |
| ATOM | 3514 | OE2 | GLU | D | 84 | −2.684 | 28.426 | 85.416 | 1.00 | 85.73 | D |
| ATOM | 3515 | C | GLU | D | 84 | −6.305 | 25.537 | 87.314 | 1.00 | 76.22 | D |
| ATOM | 3516 | O | GLU | D | 84 | −6.937 | 26.595 | 87.381 | 1.00 | 76.69 | D |
| ATOM | 3517 | N | TYR | D | 85 | −6.623 | 24.546 | 86.478 | 1.00 | 74.12 | D |
| ATOM | 3518 | CA | TYR | D | 85 | −7.746 | 24.655 | 85.550 | 1.00 | 72.24 | D |
| ATOM | 3519 | CB | TYR | D | 85 | −7.350 | 24.115 | 84.169 | 1.00 | 68.78 | D |
| ATOM | 3520 | CG | TYR | D | 85 | −6.294 | 24.961 | 83.496 | 1.00 | 66.54 | D |
| ATOM | 3521 | CD1 | TYR | D | 85 | −4.939 | 24.729 | 83.718 | 1.00 | 65.26 | D |
| ATOM | 3522 | CE1 | TYR | D | 85 | −3.965 | 25.569 | 83.180 | 1.00 | 65.00 | D |
| ATOM | 3523 | CD2 | TYR | D | 85 | −6.651 | 26.052 | 82.708 | 1.00 | 68.01 | D |
| ATOM | 3524 | CE2 | TYR | D | 85 | −5.684 | 26.900 | 82.164 | 1.00 | 67.99 | D |
| ATOM | 3525 | CZ | TYR | D | 85 | −4.345 | 26.653 | 82.409 | 1.00 | 66.05 | D |
| ATOM | 3526 | OH | TYR | D | 85 | −3.393 | 27.508 | 81.908 | 1.00 | 66.47 | D |
| ATOM | 3527 | C | TYR | D | 85 | −9.034 | 23.985 | 86.027 | 1.00 | 72.88 | D |
| ATOM | 3528 | O | TYR | D | 85 | −10.126 | 24.482 | 85.757 | 1.00 | 74.79 | D |
| ATOM | 3529 | N | VAL | D | 86 | −8.916 | 22.864 | 86.732 | 1.00 | 71.79 | D |
| ATOM | 3530 | CA | VAL | D | 86 | −10.096 | 22.170 | 87.239 | 1.00 | 70.89 | D |
| ATOM | 3531 | CB | VAL | D | 86 | −10.293 | 20.823 | 86.521 | 1.00 | 70.48 | D |
| ATOM | 3532 | CG1 | VAL | D | 86 | −11.663 | 20.242 | 86.859 | 1.00 | 69.18 | D |
| ATOM | 3533 | CG2 | VAL | D | 86 | −10.143 | 21.018 | 85.022 | 1.00 | 70.88 | D |
| ATOM | 3534 | C | VAL | D | 86 | −9.913 | 21.936 | 88.738 | 1.00 | 71.32 | D |
| ATOM | 3535 | O | VAL | D | 86 | −9.354 | 20.921 | 89.158 | 1.00 | 70.37 | D |
| ATOM | 3536 | N | ASP | D | 87 | −10.391 | 22.883 | 89.541 | 1.00 | 71.05 | D |
| ATOM | 3537 | CA | ASP | D | 87 | −10.247 | 22.801 | 90.989 | 1.00 | 71.35 | D |
| ATOM | 3538 | CB | ASP | D | 87 | −9.923 | 24.189 | 91.550 | 1.00 | 73.46 | D |
| ATOM | 3539 | CG | ASP | D | 87 | −9.162 | 24.126 | 92.865 | 1.00 | 75.27 | D |
| ATOM | 3540 | OD1 | ASP | D | 87 | −9.433 | 23.210 | 93.674 | 1.00 | 74.58 | D |
| ATOM | 3541 | OD2 | ASP | D | 87 | −8.298 | 25.003 | 93.092 | 1.00 | 76.92 | D |
| ATOM | 3542 | C | ASP | D | 87 | −11.456 | 22.235 | 91.737 | 1.00 | 70.15 | D |
| ATOM | 3543 | O | ASP | D | 87 | −12.424 | 22.947 | 91.992 | 1.00 | 68.95 | D |
| ATOM | 3544 | N | LEU | D | 88 | −11.392 | 20.958 | 92.099 | 1.00 | 70.32 | D |
| ATOM | 3545 | CA | LEU | D | 88 | −12.472 | 20.325 | 92.850 | 1.00 | 70.39 | D |
| ATOM | 3546 | CB | LEU | D | 88 | −12.599 | 18.844 | 92.472 | 1.00 | 68.68 | D |
| ATOM | 3547 | CG | LEU | D | 88 | −13.329 | 18.516 | 91.166 | 1.00 | 69.48 | D |
| ATOM | 3548 | CD1 | LEU | D | 88 | −13.192 | 17.038 | 90.844 | 1.00 | 69.02 | D |
| ATOM | 3549 | CD2 | LEU | D | 88 | −14.796 | 18.891 | 91.299 | 1.00 | 69.40 | D |
| ATOM | 3550 | C | LEU | D | 88 | −12.166 | 20.460 | 94.342 | 1.00 | 70.80 | D |
| ATOM | 3551 | O | LEU | D | 88 | −12.929 | 19.990 | 95.190 | 1.00 | 68.91 | D |
| ATOM | 3552 | N | GLU | D | 89 | −11.049 | 21.127 | 94.637 | 1.00 | 72.30 | D |
| ATOM | 3553 | CA | GLU | D | 89 | −10.573 | 21.345 | 96.003 | 1.00 | 73.10 | D |
| ATOM | 3554 | CB | GLU | D | 89 | −9.044 | 21.278 | 96.030 | 0.00 | 72.98 | D |
| ATOM | 3555 | CG | GLU | D | 89 | −8.460 | 20.022 | 95.406 | 0.00 | 72.92 | D |
| ATOM | 3556 | CD | GLU | D | 89 | −6.950 | 20.087 | 95.279 | 0.00 | 72.92 | D |
| ATOM | 3557 | OE1 | GLU | D | 89 | −6.450 | 21.016 | 94.610 | 0.00 | 72.88 | D |
| ATOM | 3558 | OE2 | GLU | D | 89 | −6.264 | 19.211 | 95.845 | 0.00 | 72.84 | D |
| ATOM | 3559 | C | GLU | D | 89 | −11.022 | 22.676 | 96.613 | 1.00 | 73.56 | D |
| ATOM | 3560 | O | GLU | D | 89 | −11.881 | 22.702 | 97.488 | 1.00 | 73.67 | D |
| ATOM | 3561 | N | ARG | D | 90 | −10.422 | 23.767 | 96.144 | 1.00 | 74.44 | D |
| ATOM | 3562 | CA | ARG | D | 90 | −10.709 | 25.123 | 96.624 | 1.00 | 75.64 | D |
| ATOM | 3563 | CB | ARG | D | 90 | −10.853 | 26.084 | 95.441 | 1.00 | 75.99 | D |
| ATOM | 3564 | CG | ARG | D | 90 | −11.060 | 27.533 | 95.851 | 1.00 | 74.12 | D |
| ATOM | 3565 | CD | ARG | D | 90 | −10.946 | 28.456 | 94.660 | 1.00 | 74.27 | D |
| ATOM | 3566 | NE | ARG | D | 90 | −11.163 | 29.851 | 95.020 | 1.00 | 77.06 | D |
| ATOM | 3567 | CZ | ARG | D | 90 | −11.101 | 30.861 | 94.157 | 1.00 | 80.00 | D |
| ATOM | 3568 | NH1 | ARG | D | 90 | −10.824 | 30.627 | 92.881 | 1.00 | 79.81 | D |
| ATOM | 3569 | NH2 | ARG | D | 90 | −11.323 | 32.104 | 94.566 | 1.00 | 79.49 | D |
| ATOM | 3570 | C | ARG | D | 90 | −11.916 | 25.278 | 97.543 | 1.00 | 76.24 | D |
| ATOM | 3571 | O | ARG | D | 90 | −11.789 | 25.811 | 98.642 | 1.00 | 75.84 | D |
| ATOM | 3572 | N | GLU | D | 91 | −13.088 | 24.841 | 97.091 | 1.00 | 77.46 | D |
| ATOM | 3573 | CA | GLU | D | 91 | −14.290 | 24.927 | 97.917 | 1.00 | 78.92 | D |
| ATOM | 3574 | CB | GLU | D | 91 | −15.072 | 26.211 | 97.620 | 1.00 | 79.99 | D |
| ATOM | 3575 | CG | GLU | D | 91 | −15.651 | 26.860 | 98.874 | 1.00 | 82.64 | D |
| ATOM | 3576 | CD | GLU | D | 91 | −16.852 | 27.745 | 98.596 | 1.00 | 84.44 | D |
| ATOM | 3577 | OE1 | GLU | D | 91 | −17.949 | 27.198 | 98.358 | 1.00 | 83.55 | D |
| ATOM | 3578 | OE2 | GLU | D | 91 | −16.698 | 28.986 | 98.612 | 1.00 | 87.28 | D |
| ATOM | 3579 | C | GLU | D | 91 | −15.207 | 23.721 | 97.715 | 1.00 | 78.91 | D |
| ATOM | 3580 | O | GLU | D | 91 | −15.710 | 23.484 | 96.615 | 1.00 | 80.34 | D |
| ATOM | 3581 | N | ALA | D | 92 | −15.421 | 22.964 | 98.787 | 1.00 | 77.60 | D |
| ATOM | 3582 | CA | ALA | D | 92 | −16.276 | 21.781 | 98.743 | 1.00 | 74.11 | D |
| ATOM | 3583 | CB | ALA | D | 92 | −16.433 | 21.207 | 100.146 | 1.00 | 73.59 | D |
| ATOM | 3584 | C | ALA | D | 92 | −17.649 | 22.100 | 98.155 | 1.00 | 71.48 | D |
| ATOM | 3585 | O | ALA | D | 92 | −18.226 | 23.154 | 98.432 | 1.00 | 69.46 | D |
| ATOM | 3586 | N | GLY | D | 93 | −18.167 | 21.185 | 97.341 | 1.00 | 69.18 | D |
| ATOM | 3587 | CA | GLY | D | 93 | −19.474 | 21.391 | 96.742 | 1.00 | 66.60 | D |
| ATOM | 3588 | C | GLY | D | 93 | −19.451 | 22.032 | 95.364 | 1.00 | 64.95 | D |
| ATOM | 3589 | O | GLY | D | 93 | −20.468 | 22.052 | 94.668 | 1.00 | 65.18 | D |
| ATOM | 3590 | N | LYS | D | 94 | −18.301 | 22.563 | 94.962 | 1.00 | 62.36 | D |
| ATOM | 3591 | CA | LYS | D | 94 | −18.197 | 23.187 | 93.652 | 1.00 | 60.23 | D |
| ATOM | 3592 | CB | LYS | D | 94 | −18.712 | 24.624 | 93.718 | 1.00 | 60.64 | D |
| ATOM | 3593 | CG | LYS | D | 94 | −17.960 | 25.572 | 94.632 | 1.00 | 57.30 | D |
| ATOM | 3594 | CD | LYS | D | 94 | −18.823 | 26.808 | 94.805 | 1.00 | 59.27 | D |
| ATOM | 3595 | CE | LYS | D | 94 | −18.107 | 27.951 | 95.475 | 1.00 | 63.07 | D |
| ATOM | 3596 | NZ | LYS | D | 94 | −19.057 | 29.085 | 95.676 | 1.00 | 66.18 | D |
| ATOM | 3597 | C | LYS | D | 94 | −16.800 | 23.157 | 93.039 | 1.00 | 58.84 | D |
| ATOM | 3598 | O | LYS | D | 94 | 15.790 | 23.055 | 93.746 | 1.00 | 59.83 | D |
| ATOM | 3599 | N | VAL | D | 95 | −16.753 | 23.241 | 91.712 | 1.00 | 53.98 | D |
| ATOM | 3600 | CA | VAL | D | 95 | −15.486 | 23.213 | 90.996 | 1.00 | 49.53 | D |
| ATOM | 3601 | CB | VAL | D | 95 | −15.523 | 22.187 | 89.836 | 1.00 | 48.68 | D |
| ATOM | 3602 | CG1 | VAL | D | 95 | −16.599 | 22.565 | 88.841 | 1.00 | 46.34 | D |
| ATOM | 3603 | CG2 | VAL | D | 95 | −14.168 | 22.117 | 89.151 | 1.00 | 48.81 | D |
| ATOM | 3604 | C | VAL | D | 95 | −15.135 | 24.572 | 90.426 | 1.00 | 47.53 | D |
| ATOM | 3605 | O | VAL | D | 95 | −16.014 | 25.319 | 89.996 | 1.00 | 51.62 | D |
| ATOM | 3606 | N | TYR | D | 96 | −13.850 | 24.903 | 90.444 | 1.00 | 44.65 | D |
| ATOM | 3607 | CA | TYR | D | 96 | −13.391 | 26.162 | 89.878 | 1.00 | 45.94 | D |
| ATOM | 3608 | CB | TYR | D | 96 | −12.442 | 26.877 | 90.842 | 1.00 | 47.61 | D |
| ATOM | 3609 | CG | TYR | D | 96 | −13.194 | 27.708 | 91.863 | 1.00 | 52.02 | D |
| ATOM | 3610 | CD1 | TYR | D | 96 | −13.735 | 27.127 | 93.011 | 1.00 | 52.88 | D |
| ATOM | 3611 | CE1 | TYR | D | 96 | −14.475 | 27.883 | 93.922 | 1.00 | 55.65 | D |
| ATOM | 3612 | CD2 | TYR | D | 96 | −13.411 | 29.071 | 91.653 | 1.00 | 53.89 | D |
| ATOM | 3613 | CE2 | TYR | D | 96 | −14.149 | 29.835 | 92.553 | 1.00 | 56.72 | D |
| ATOM | 3614 | CZ | TYR | D | 96 | −14.679 | 29.237 | 93.688 | 1.00 | 58.24 | D |
| ATOM | 3615 | OH | TYR | D | 96 | −15.412 | 29.996 | 94.580 | 1.00 | 60.71 | D |
| ATOM | 3616 | C | TYR | D | 96 | −12.716 | 25.854 | 88.541 | 1.00 | 46.23 | D |
| ATOM | 3617 | O | TYR | D | 96 | −11.753 | 25.084 | 88.477 | 1.00 | 47.95 | D |
| ATOM | 3618 | N | LEU | D | 97 | −13.244 | 26.448 | 87.474 | 1.00 | 43.48 | D |
| ATOM | 3619 | CA | LEU | D | 97 | −12.741 | 26.206 | 86.127 | 1.00 | 38.90 | D |
| ATOM | 3620 | CB | LEU | D | 97 | −13.891 | 25.712 | 85.252 | 1.00 | 38.80 | D |
| ATOM | 3621 | CG | LEU | D | 97 | −14.621 | 24.472 | 85.759 | 1.00 | 38.57 | D |
| ATOM | 3622 | CD1 | LEU | D | 97 | −15.979 | 24.346 | 85.092 | 1.00 | 35.65 | D |
| ATOM | 3623 | CD2 | LEU | D | 97 | −13.759 | 23.248 | 85.486 | 1.00 | 40.92 | D |
| ATOM | 3624 | C | LEU | D | 97 | −12.096 | 27.420 | 85.472 | 1.00 | 37.32 | D |
| ATOM | 3625 | O | LEU | D | 97 | −12.542 | 28.553 | 85.667 | 1.00 | 35.97 | D |
| ATOM | 3626 | N | LYS | D | 98 | −11.051 | 27.158 | 84.686 | 1.00 | 36.83 | D |
| ATOM | 3627 | CA | LYS | D | 98 | −10.302 | 28.182 | 83.949 | 1.00 | 38.18 | D |
| ATOM | 3628 | CB | LYS | D | 98 | −8.911 | 28.356 | 84.563 | 1.00 | 40.93 | D |
| ATOM | 3629 | CG | LYS | D | 98 | −8.032 | 29.432 | 83.935 | 1.00 | 46.34 | D |
| ATOM | 3630 | CD | LYS | D | 98 | −6.617 | 29.361 | 84.526 | 1.00 | 52.94 | D |
| ATOM | 3631 | CE | LYS | D | 98 | −5.680 | 30.454 | 84.008 | 1.00 | 58.30 | D |
| ATOM | 3632 | NZ | LYS | D | 98 | −5.793 | 31.753 | 84.752 | 1.00 | 61.05 | D |
| ATOM | 3633 | C | LYS | D | 98 | −10.172 | 27.667 | 82.511 | 1.00 | 36.19 | D |
| ATOM | 3634 | O | LYS | D | 98 | −9.898 | 26.487 | 82.289 | 1.00 | 33.97 | D |
| ATOM | 3635 | N | ALA | D | 99 | −10.379 | 28.533 | 81.530 | 1.00 | 34.05 | D |
| ATOM | 3636 | CA | ALA | D | 99 | −10.276 | 28.082 | 80.144 | 1.00 | 35.32 | D |
| ATOM | 3637 | CB | ALA | D | 99 | −11.560 | 27.352 | 79.751 | 1.00 | 33.03 | D |
| ATOM | 3638 | C | ALA | D | 99 | −9.962 | 29.182 | 79.117 | 1.00 | 34.03 | D |
| ATOM | 3639 | O | ALA | D | 99 | −10.762 | 30.084 | 78.899 | 1.00 | 35.09 | D |
| ATOM | 3640 | N | PRO | D | 100 | −8.779 | 29.115 | 78.488 | 1.00 | 32.07 | D |
| ATOM | 3641 | CD | PRO | D | 100 | −7.706 | 28.189 | 78.874 | 1.00 | 33.05 | D |
| ATOM | 3642 | CA | PRO | D | 100 | −8.302 | 30.065 | 77.469 | 1.00 | 36.09 | D |
| ATOM | 3643 | CB | PRO | D | 100 | −6.785 | 29.860 | 77.464 | 1.00 | 32.40 | D |
| ATOM | 3644 | CG | PRO | D | 100 | −6.506 | 29.052 | 78.713 | 1.00 | 37.33 | D |
| ATOM | 3645 | C | PRO | D | 100 | −8.887 | 29.748 | 76.080 | 1.00 | 34.53 | D |
| ATOM | 3646 | O | PRO | D | 100 | −9.092 | 28.585 | 75.738 | 1.00 | 37.58 | D |
| ATOM | 3647 | N | MET | D | 101 | −9.145 | 30.779 | 75.285 | 1.00 | 31.05 | D |
| ATOM | 3648 | CA | MET | D | 101 | −9.681 | 30.586 | 73.942 | 1.00 | 30.79 | D |
| ATOM | 3649 | CB | MET | D | 101 | −11.141 | 30.138 | 74.011 | 1.00 | 31.98 | D |
| ATOM | 3650 | CG | MET | D | 101 | −12.097 | 31.266 | 74.441 | 1.00 | 34.04 | D |
| ATOM | 3651 | SD | MET | D | 101 | −13.672 | 30.719 | 75.159 | 1.00 | 38.91 | D |
| ATOM | 3652 | CE | MET | D | 101 | −14.781 | 31.803 | 74.365 | 1.00 | 41.89 | D |
| ATOM | 3653 | C | MET | D | 101 | −9.630 | 31.909 | 73.205 | 1.00 | 27.96 | D |
| ATOM | 3654 | O | MET | D | 101 | −9.347 | 32.948 | 73.790 | 1.00 | 32.03 | D |
| ATOM | 3655 | N | ILE | D | 102 | −9.877 | 31.877 | 71.913 | 1.00 | 24.31 | D |
| ATOM | 3656 | CA | ILE | D | 102 | −9.936 | 33.121 | 71.183 | 1.00 | 25.32 | D |
| ATOM | 3657 | CB | ILE | D | 102 | −9.085 | 33.113 | 69.883 | 1.00 | 25.82 | D |
| ATOM | 3658 | CG2 | ILE | D | 102 | −9.330 | 34.404 | 69.098 | 1.00 | 20.09 | D |
| ATOM | 3659 | CG1 | ILE | D | 102 | −7.598 | 33.022 | 70.228 | 1.00 | 22.99 | D |
| ATOM | 3660 | CD1 | ILE | D | 102 | −6.696 | 32.990 | 69.040 | 1.00 | 23.58 | D |
| ATOM | 3661 | C | ILE | D | 102 | −11.411 | 33.211 | 70.839 | 1.00 | 27.26 | D |
| ATOM | 3662 | O | ILE | D | 102 | −11.919 | 32.415 | 70.045 | 1.00 | 27.11 | D |
| ATOM | 3663 | N | LEU | D | 103 | −12.101 | 34.147 | 71.483 | 1.00 | 26.63 | D |
| ATOM | 3664 | CA | LEU | D | 103 | −13.523 | 34.360 | 71.264 | 1.00 | 24.36 | D |
| ATOM | 3665 | CB | LEU | D | 103 | −14.222 | 34.556 | 72.611 | 1.00 | 25.04 | D |
| ATOM | 3666 | CG | LEU | D | 103 | −15.740 | 34.769 | 72.628 | 1.00 | 31.42 | D |
| ATOM | 3667 | CD1 | LEU | D | 103 | −16.409 | 33.719 | 71.765 | 1.00 | 25.36 | D |
| ATOM | 3668 | CD2 | LEU | D | 103 | −16.256 | 34.713 | 74.065 | 1.00 | 24.28 | D |
| ATOM | 3669 | C | LEU | D | 103 | −13.724 | 35.579 | 70.372 | 1.00 | 23.28 | D |
| ATOM | 3670 | O | LEU | D | 103 | −13.314 | 36.666 | 70.710 | 1.00 | 25.64 | D |
| ATOM | 3671 | N | ASN | D | 104 | −14.341 | 35.382 | 69.215 | 1.00 | 28.92 | D |
| ATOM | 3672 | CA | ASN | D | 104 | −14.590 | 36.466 | 68.262 | 1.00 | 27.55 | D |
| ATOM | 3673 | CB | ASN | D | 104 | −15.831 | 37.274 | 68.666 | 1.00 | 26.75 | D |
| ATOM | 3674 | CG | ASN | D | 104 | −17.112 | 36.449 | 68.625 | 1.00 | 30.77 | D |
| ATOM | 3675 | OD1 | ASN | D | 104 | −17.230 | 35.507 | 67.838 | 1.00 | 25.46 | D |
| ATOM | 3676 | ND2 | ASN | D | 104 | −18.085 | 36.811 | 69.468 | 1.00 | 26.22 | D |
| ATOM | 3677 | C | ASN | D | 104 | −13.412 | 37.422 | 68.064 | 1.00 | 28.72 | D |
| ATOM | 3678 | O | ASN | D | 104 | −13.545 | 38.634 | 68.237 | 1.00 | 27.80 | D |
| ATOM | 3679 | N | GLY | D | 105 | −12.258 | 36.868 | 67.704 | 1.00 | 32.10 | D |
| ATOM | 3680 | CA | GLY | D | 105 | −11.080 | 37.682 | 67.449 | 1.00 | 33.88 | D |
| ATOM | 3681 | C | GLY | D | 105 | −10.254 | 38.176 | 68.626 | 1.00 | 36.77 | D |
| ATOM | 3682 | O | GLY | D | 105 | −9.242 | 38.848 | 68.412 | 1.00 | 37.99 | D |
| ATOM | 3683 | N | VAL | D | 106 | −10.649 | 37.863 | 69.860 | 1.00 | 34.93 | D |
| ATOM | 3684 | CA | VAL | D | 106 | −9.873 | 38.331 | 70.999 | 1.00 | 34.41 | D |
| ATOM | 3685 | CB | VAL | D | 106 | −10.565 | 39.562 | 71.682 | 1.00 | 36.14 | D |
| ATOM | 3686 | CG1 | VAL | D | 106 | −11.881 | 39.863 | 71.004 | 1.00 | 27.37 | D |
| ATOM | 3687 | CG2 | VAL | D | 106 | −10.734 | 39.320 | 73.183 | 1.00 | 33.28 | D |
| ATOM | 3688 | C | VAL | D | 106 | −9.521 | 37.272 | 72.047 | 1.00 | 34.42 | D |
| ATOM | 3689 | O | VAL | D | 106 | −10.366 | 36.505 | 72.498 | 1.00 | 34.71 | D |
| ATOM | 3690 | N | CYS | D | 107 | −8.253 | 37.227 | 72.427 | 1.00 | 34.99 | D |
| ATOM | 3691 | CA | CYS | D | 107 | −7.820 | 36.260 | 73.418 | 1.00 | 35.43 | D |
| ATOM | 3692 | CB | CYS | D | 107 | −6.307 | 36.322 | 73.573 | 1.00 | 36.19 | D |
| ATOM | 3693 | SG | CYS | D | 107 | −5.431 | 35.832 | 72.060 | 1.00 | 37.91 | D |
| ATOM | 3694 | C | CYS | D | 107 | −8.501 | 36.519 | 74.750 | 1.00 | 34.04 | D |
| ATOM | 3695 | O | CYS | D | 107 | −8.446 | 37.618 | 75.283 | 1.00 | 39.69 | D |
| ATOM | 3696 | N | VAL | D | 108 | −9.162 | 35.502 | 75.279 | 1.00 | 30.89 | D |
| ATOM | 3697 | CA | VAL | D | 108 | −9.844 | 35.642 | 76.544 | 1.00 | 27.72 | D |
| ATOM | 3698 | CB | VAL | D | 108 | −11.359 | 35.863 | 76.341 | 1.00 | 28.16 | D |
| ATOM | 3699 | CG1 | VAL | D | 108 | −11.586 | 37.066 | 75.455 | 1.00 | 29.68 | D |
| ATOM | 3700 | CG2 | VAL | D | 108 | −12.004 | 34.620 | 75.752 | 1.00 | 23.27 | D |
| ATOM | 3701 | C | VAL | D | 108 | −9.635 | 34.400 | 77.402 | 1.00 | 31.97 | D |
| ATOM | 3702 | O | VAL | D | 108 | −9.074 | 33.400 | 76.949 | 1.00 | 35.59 | D |
| ATOM | 3703 | N | ILE | D | 109 | −10.060 | 34.488 | 78.656 | 1.00 | 31.10 | D |
| ATOM | 3704 | CA | ILE | D | 109 | −9.984 | 33.371 | 79.579 | 1.00 | 32.35 | D |
| ATOM | 3705 | CB | ILE | D | 109 | −8.895 | 33.536 | 80.647 | 1.00 | 32.24 | D |
| ATOM | 3706 | CG2 | ILE | D | 109 | −8.932 | 32.340 | 81.575 | 1.00 | 35.08 | D |
| ATOM | 3707 | CG1 | ILE | D | 109 | −7.508 | 33.607 | 80.004 | 1.00 | 37.56 | D |
| ATOM | 3708 | CD1 | ILE | D | 109 | −6.355 | 33.595 | 81.003 | 1.00 | 39.93 | D |
| ATOM | 3709 | C | ILE | D | 109 | −11.325 | 33.324 | 80.278 | 1.00 | 34.72 | D |
| ATOM | 3710 | O | ILE | D | 109 | −11.819 | 34.333 | 80.778 | 1.00 | 35.13 | D |
| ATOM | 3711 | N | TRP | D | 110 | −11.921 | 32.148 | 80.298 | 1.00 | 34.27 | D |
| ATOM | 3712 | CA | TRP | D | 110 | −13.214 | 31.982 | 80.912 | 1.00 | 35.43 | D |
| ATOM | 3713 | CB | TRP | D | 110 | −14.053 | 31.052 | 80.038 | 1.00 | 33.82 | D |
| ATOM | 3714 | CG | TRP | D | 110 | −15.440 | 30.806 | 80.505 | 1.00 | 33.77 | D |
| ATOM | 3715 | CD2 | TRP | D | 110 | −15.843 | 29.940 | 81.563 | 1.00 | 33.62 | D |
| ATOM | 3716 | CE2 | TRP | D | 110 | −17.254 | 29.996 | 81.633 | 1.00 | 35.75 | D |
| ATOM | 3717 | CE3 | TRP | D | 110 | −15.151 | 29.118 | 82.463 | 1.00 | 36.28 | D |
| ATOM | 3718 | CD1 | TRP | D | 110 | −16.587 | 31.340 | 79.989 | 1.00 | 36.92 | D |
| ATOM | 3719 | NE1 | TRP | D | 110 | −17.679 | 30.860 | 80.659 | 1.00 | 35.50 | D |
| ATOM | 3720 | CZ2 | TRP | D | 110 | −17.992 | 29.257 | 82.573 | 1.00 | 36.90 | D |
| ATOM | 3721 | CZ3 | TRP | D | 110 | −15.889 | 28.380 | 83.406 | 1.00 | 36.06 | D |
| ATOM | 3722 | CH2 | TRP | D | 110 | −17.293 | 28.458 | 83.447 | 1.00 | 35.00 | D |
| ATOM | 3723 | C | TRP | D | 110 | −12.962 | 31.370 | 82.280 | 1.00 | 39.79 | D |
| ATOM | 3724 | O | TRP | D | 110 | −12.372 | 30.287 | 82.387 | 1.00 | 40.95 | D |
| ATOM | 3725 | N | LYS | D | 111 | −13.376 | 32.082 | 83.325 | 1.00 | 40.28 | D |
| ATOM | 3726 | CA | LYS | D | 111 | −13.213 | 31.606 | 84.695 | 1.00 | 41.34 | D |
| ATOM | 3727 | CB | LYS | D | 111 | −12.256 | 32.492 | 85.492 | 1.00 | 41.40 | D |
| ATOM | 3728 | CG | LYS | D | 111 | −10.803 | 32.195 | 85.286 | 1.00 | 45.87 | D |
| ATOM | 3729 | CD | LYS | D | 111 | −9.957 | 33.355 | 85.757 | 1.00 | 52.47 | D |
| ATOM | 3730 | CE | LYS | D | 111 | −8.483 | 33.108 | 85.481 | 1.00 | 59.00 | D |
| ATOM | 3731 | NZ | LYS | D | 111 | −7.695 | 34.380 | 85.446 | 1.00 | 60.92 | D |
| ATOM | 3732 | C | LYS | D | 111 | −14.559 | 31.645 | 85.358 | 1.00 | 41.65 | D |
| ATOM | 3733 | O | LYS | D | 111 | −15.347 | 32.566 | 85.136 | 1.00 | 42.13 | D |
| ATOM | 3734 | N | GLY | D | 112 | −14.815 | 30.634 | 86.171 | 1.00 | 41.13 | D |
| ATOM | 3735 | CA | GLY | D | 112 | −16.066 | 30.560 | 86.889 | 1.00 | 42.25 | D |
| ATOM | 3736 | C | GLY | D | 112 | −16.059 | 29.313 | 87.743 | 1.00 | 43.94 | D |
| ATOM | 3737 | O | GLY | D | 112 | −15.103 | 28.528 | 87.726 | 1.00 | 44.53 | D |
| ATOM | 3738 | N | TRP | D | 113 | −17.126 | 29.132 | 88.504 | 1.00 | 44.09 | D |
| ATOM | 3739 | CA | TRP | D | 113 | −17.263 | 27.958 | 89.346 | 1.00 | 45.05 | D |
| ATOM | 3740 | CB | TRP | D | 113 | −17.030 | 28.307 | 90.818 | 1.00 | 44.11 | D |
| ATOM | 3741 | CG | TRP | D | 113 | −17.910 | 29.411 | 91.305 | 1.00 | 44.80 | D |
| ATOM | 3742 | CD2 | TRP | D | 113 | −19.263 | 29.291 | 91.775 | 1.00 | 46.20 | D |
| ATOM | 3743 | CE2 | TRP | D | 113 | −19.714 | 30.594 | 92.083 | 1.00 | 46.11 | D |
| ATOM | 3744 | CE3 | TRP | D | 113 | −20.138 | 28.208 | 91.963 | 1.00 | 45.98 | D |
| ATOM | 3745 | CD1 | TRP | D | 113 | −17.606 | 30.740 | 91.349 | 1.00 | 43.74 | D |
| ATOM | 3746 | NE1 | TRP | D | 113 | −18.684 | 31.457 | 91.813 | 1.00 | 44.74 | D |
| ATOM | 3747 | CZ2 | TRP | D | 113 | −21.007 | 30.848 | 92.573 | 1.00 | 44.69 | D |
| ATOM | 3748 | CZ3 | TRP | D | 113 | −21.431 | 28.461 | 92.452 | 1.00 | 43.10 | D |
| ATOM | 3749 | CH2 | TRP | D | 113 | −21.845 | 29.772 | 92.749 | 1.00 | 45.47 | D |
| ATOM | 3750 | C | TRP | D | 113 | −18.680 | 27.459 | 89.153 | 1.00 | 45.09 | D |
| ATOM | 3751 | O | TRP | D | 113 | −19.575 | 28.216 | 88.779 | 1.00 | 43.43 | D |
| ATOM | 3752 | N | ILE | D | 114 | −18.890 | 26.180 | 89.403 | 1.00 | 46.97 | D |
| ATOM | 3753 | CA | ILE | D | 114 | −20.217 | 25.625 | 89.247 | 1.00 | 49.55 | D |
| ATOM | 3754 | CB | ILE | D | 114 | −20.337 | 24.868 | 87.887 | 1.00 | 49.68 | D |
| ATOM | 3755 | CG2 | ILE | D | 114 | −21.468 | 23.835 | 87.926 | 1.00 | 45.70 | D |
| ATOM | 3756 | CG1 | ILE | D | 114 | −20.538 | 25.905 | 86.775 | 1.00 | 49.94 | D |
| ATOM | 3757 | CD1 | ILE | D | 114 | −21.049 | 25.352 | 85.475 | 1.00 | 55.34 | D |
| ATOM | 3758 | C | ILE | D | 114 | −20.572 | 24.727 | 90.417 | 1.00 | 50.84 | D |
| ATOM | 3759 | O | ILE | D | 114 | −19.759 | 23.916 | 90.869 | 1.00 | 51.20 | D |
| ATOM | 3760 | N | ASP | D | 115 | −21.787 | 24.905 | 90.923 | 1.00 | 51.47 | D |
| ATOM | 3761 | CA | ASP | D | 115 | −22.266 | 24.108 | 92.040 | 1.00 | 51.83 | D |
| ATOM | 3762 | CB | ASP | D | 115 | −23.589 | 24.679 | 92.561 | 1.00 | 55.34 | D |
| ATOM | 3763 | CG | ASP | D | 115 | −23.958 | 24.138 | 93.928 | 1.00 | 57.00 | D |
| ATOM | 3764 | OD1 | ASP | D | 115 | −24.094 | 22.904 | 94.073 | 1.00 | 58.30 | D |
| ATOM | 3765 | OD2 | ASP | D | 115 | −24.110 | 24.954 | 94.860 | 1.00 | 59.07 | D |
| ATOM | 3766 | C | ASP | D | 115 | −22.474 | 22.692 | 91.515 | 1.00 | 50.41 | D |
| ATOM | 3767 | O | ASP | D | 115 | −23.304 | 22.471 | 90.629 | 1.00 | 46.97 | D |
| ATOM | 3768 | N | LEU | D | 116 | −21.710 | 21.744 | 92.054 | 1.00 | 50.02 | D |
| ATOM | 3769 | CA | LEU | D | 116 | −21.799 | 20.347 | 91.637 | 1.00 | 50.48 | D |
| ATOM | 3770 | CB | LEU | D | 116 | −20.753 | 19.519 | 92.373 | 1.00 | 49.48 | D |
| ATOM | 3771 | CG | LEU | D | 116 | −19.315 | 19.980 | 92.133 | 1.00 | 51.21 | D |
| ATOM | 3772 | CD1 | LEU | D | 116 | −18.352 | 19.231 | 93.042 | 1.00 | 46.84 | D |
| ATOM | 3773 | CD2 | LEU | D | 116 | −18.972 | 19.755 | 90.669 | 1.00 | 52.30 | D |
| ATOM | 3774 | C | LEU | D | 116 | −23.184 | 19.768 | 91.888 | 1.00 | 51.98 | D |
| ATOM | 3775 | O | LEU | D | 116 | −23.534 | 18.711 | 91.363 | 1.00 | 54.23 | D |
| ATOM | 3776 | N | GLN | D | 117 | −23.973 | 20.476 | 92.687 | 1.00 | 53.27 | D |
| ATOM | 3777 | CA | GLN | D | 117 | −25.324 | 20.049 | 93.020 | 1.00 | 54.64 | D |
| ATOM | 3778 | CB | GLN | D | 117 | −25.636 | 20.448 | 94.469 | 1.00 | 60.59 | D |
| ATOM | 3779 | CG | GLN | D | 117 | −26.676 | 19.601 | 95.192 | 1.00 | 67.61 | D |
| ATOM | 3780 | CD | GLN | D | 117 | −26.110 | 18.283 | 95.693 | 1.00 | 73.15 | D |
| ATOM | 3781 | OE1 | GLN | D | 117 | −25.882 | 17.354 | 94.915 | 1.00 | 76.27 | D |
| ATOM | 3782 | NE2 | GLN | D | 117 | −25.868 | 18.201 | 97.001 | 1.00 | 73.94 | D |
| ATOM | 3783 | C | GLN | D | 117 | −26.307 | 20.741 | 92.069 | 1.00 | 53.30 | D |
| ATOM | 3784 | O | GLN | D | 117 | −27.051 | 20.091 | 91.337 | 1.00 | 51.37 | D |
| ATOM | 3785 | N | ARG | D | 118 | −26.291 | 22.071 | 92.086 | 1.00 | 50.44 | D |
| ATOM | 3786 | CA | ARG | D | 118 | −27.178 | 22.864 | 91.252 | 1.00 | 49.79 | D |
| ATOM | 3787 | CB | ARG | D | 118 | −27.186 | 24.316 | 91.725 | 1.00 | 54.97 | D |
| ATOM | 3788 | CG | ARG | D | 118 | −27.701 | 24.547 | 93.130 | 1.00 | 56.83 | D |
| ATOM | 3789 | CD | ARG | D | 118 | −27.940 | 26.032 | 93.357 | 1.00 | 60.48 | D |
| ATOM | 3790 | NE | ARG | D | 118 | −28.831 | 26.591 | 92.340 | 1.00 | 64.97 | D |
| ATOM | 3791 | CZ | ARG | D | 118 | −29.419 | 27.780 | 92.428 | 1.00 | 68.50 | D |
| ATOM | 3792 | NH1 | ARG | D | 118 | −29.214 | 28.545 | 93.493 | 1.00 | 72.02 | D |
| ATOM | 3793 | NH2 | ARG | D | 118 | −30.214 | 28.205 | 91.454 | 1.00 | 68.85 | D |
| ATOM | 3794 | C | ARG | D | 118 | −26.797 | 22.843 | 89.782 | 1.00 | 48.52 | D |
| ATOM | 3795 | O | ARG | D | 118 | −27.654 | 23.017 | 88.913 | 1.00 | 46.76 | D |
| ATOM | 3796 | N | LEU | D | 119 | −25.505 | 22.651 | 89.515 | 1.00 | 47.25 | D |
| ATOM | 3797 | CA | LEU | D | 119 | −24.965 | 22.619 | 88.159 | 1.00 | 45.11 | D |
| ATOM | 3798 | CB | LEU | D | 119 | −25.704 | 21.595 | 87.291 | 1.00 | 40.84 | D |
| ATOM | 3799 | CG | LEU | D | 119 | −25.451 | 20.123 | 87.635 | 1.00 | 37.84 | D |
| ATOM | 3800 | CD1 | LEU | D | 119 | −26.184 | 19.256 | 86.637 | 1.00 | 35.52 | D |
| ATOM | 3801 | CD2 | LEU | D | 119 | −23.951 | 19.819 | 87.606 | 1.00 | 38.02 | D |
| ATOM | 3802 | C | LEU | D | 119 | −24.999 | 23.982 | 87.489 | 1.00 | 46.38 | D |
| ATOM | 3803 | O | LEU | D | 119 | −25.043 | 24.089 | 86.269 | 1.00 | 49.62 | D |
| ATOM | 3804 | N | ASP | D | 120 | −24.990 | 25.028 | 88.300 | 1.00 | 48.09 | D |
| ATOM | 3805 | CA | ASP | D | 120 | −24.976 | 26.388 | 87.790 | 1.00 | 48.62 | D |
| ATOM | 3806 | CB | ASP | D | 120 | −26.382 | 27.003 | 87.822 | 1.00 | 53.45 | D |
| ATOM | 3807 | CG | ASP | D | 120 | −26.810 | 27.438 | 89.212 | 1.00 | 59.98 | D |
| ATOM | 3808 | OD1 | ASP | D | 120 | −26.532 | 26.713 | 90.186 | 1.00 | 63.49 | D |
| ATOM | 3809 | OD2 | ASP | D | 120 | −27.439 | 28.508 | 89.329 | 1.00 | 64.29 | D |
| ATOM | 3810 | C | ASP | D | 120 | −24.031 | 27.122 | 88.725 | 1.00 | 46.78 | D |
| ATOM | 3811 | O | ASP | D | 120 | −23.569 | 26.543 | 89.716 | 1.00 | 44.23 | D |
| ATOM | 3812 | N | GLY | D | 121 | −23.722 | 28.374 | 88.413 | 1.00 | 44.48 | D |
| ATOM | 3813 | CA | GLY | D | 121 | −22.823 | 29.122 | 89.268 | 1.00 | 43.22 | D |
| ATOM | 3814 | C | GLY | D | 121 | −22.493 | 30.513 | 88.773 | 1.00 | 44.31 | D |
| ATOM | 3815 | O | GLY | D | 121 | −23.302 | 31.178 | 88.122 | 1.00 | 41.81 | D |
| ATOM | 3816 | N | MET | D | 122 | −21.283 | 30.955 | 89.089 | 1.00 | 44.34 | D |
| ATOM | 3817 | CA | MET | D | 122 | −20.840 | 32.275 | 88.683 | 1.00 | 45.06 | D |
| ATOM | 3818 | CB | MET | D | 122 | −20.545 | 33.145 | 89.919 | 1.00 | 46.43 | D |
| ATOM | 3819 | CG | MET | D | 122 | −21.741 | 33.395 | 90.812 | 1.00 | 42.23 | D |
| ATOM | 3820 | SD | MET | D | 122 | −23.133 | 34.090 | 89.909 | 1.00 | 49.30 | D |
| ATOM | 3821 | CE | MET | D | 122 | −22.806 | 35.846 | 90.054 | 1.00 | 44.68 | D |
| ATOM | 3822 | C | MET | D | 122 | −19.593 | 32.176 | 87.826 | 1.00 | 43.84 | D |
| ATOM | 3823 | O | MET | D | 122 | −18.706 | 31.366 | 88.097 | 1.00 | 42.51 | D |
| ATOM | 3824 | N | GLY | D | 123 | −19.527 | 33.004 | 86.788 | 1.00 | 42.95 | D |
| ATOM | 3825 | CA | GLY | D | 123 | −18.359 | 32.996 | 85.931 | 1.00 | 41.94 | D |
| ATOM | 3826 | C | GLY | D | 123 | −18.288 | 34.230 | 85.066 | 1.00 | 41.98 | D |
| ATOM | 3827 | O | GLY | D | 123 | −19.227 | 35.023 | 85.028 | 1.00 | 45.32 | D |
| ATOM | 3828 | N | CYS | D | 124 | −17.172 | 34.399 | 84.368 | 1.00 | 40.77 | D |
| ATOM | 3829 | CA | CYS | D | 124 | −17.010 | 35.544 | 83.486 | 1.00 | 40.42 | D |
| ATOM | 3830 | CB | CYS | D | 124 | −16.814 | 36.811 | 84.309 | 1.00 | 42.05 | D |
| ATOM | 3831 | SG | CYS | D | 124 | −15.217 | 36.821 | 85.174 | 1.00 | 53.39 | D |
| ATOM | 3832 | C | CYS | D | 124 | −15.819 | 35.391 | 82.548 | 1.00 | 38.93 | D |
| ATOM | 3833 | O | CYS | D | 124 | −15.023 | 34.464 | 82.661 | 1.00 | 36.29 | D |
| ATOM | 3834 | N | LEU | D | 125 | −15.705 | 36.341 | 81.636 | 1.00 | 39.37 | D |
| ATOM | 3835 | CA | LEU | D | 125 | −14.619 | 36.380 | 80.683 | 1.00 | 41.19 | D |
| ATOM | 3836 | CB | LEU | D | 125 | −15.143 | 36.861 | 79.331 | 1.00 | 38.10 | D |
| ATOM | 3837 | CG | LEU | D | 125 | −15.210 | 35.876 | 78.169 | 1.00 | 42.37 | D |
| ATOM | 3838 | CD1 | LEU | D | 125 | −15.362 | 34.440 | 78.669 | 1.00 | 42.60 | D |
| ATOM | 3839 | CD2 | LEU | D | 125 | −16.363 | 36.278 | 77.276 | 1.00 | 37.40 | D |
| ATOM | 3840 | C | LEU | D | 125 | −13.560 | 37.351 | 81.196 | 1.00 | 44.98 | D |
| ATOM | 3841 | O | LEU | D | 125 | −13.877 | 38.302 | 81.910 | 1.00 | 43.58 | D |
| ATOM | 3842 | N | GLU | D | 126 | −12.309 | 37.096 | 80.816 | 1.00 | 47.79 | D |
| ATOM | 3843 | CA | GLU | D | 126 | −11.161 | 37.920 | 81.180 | 1.00 | 47.99 | D |
| ATOM | 3844 | CB | GLU | D | 126 | −10.339 | 37.252 | 82.277 | 1.00 | 50.77 | D |
| ATOM | 3845 | CG | GLU | D | 126 | −10.713 | 37.601 | 83.679 | 1.00 | 57.05 | D |
| ATOM | 3846 | CD | GLU | D | 126 | −9.924 | 36.784 | 84.677 | 1.00 | 62.65 | D |
| ATOM | 3847 | OE1 | GLU | D | 126 | −8.681 | 36.739 | 84.554 | 1.00 | 62.53 | D |
| ATOM | 3848 | OE2 | GLU | D | 126 | −10.548 | 36.188 | 85.582 | 1.00 | 68.55 | D |
| ATOM | 3849 | C | GLU | D | 126 | −10.262 | 38.042 | 79.965 | 1.00 | 48.41 | D |
| ATOM | 3850 | O | GLU | D | 126 | −9.948 | 37.036 | 79.330 | 1.00 | 47.59 | D |
| ATOM | 3851 | N | PHE | D | 127 | −9.837 | 39.260 | 79.647 | 1.00 | 48.29 | D |
| ATOM | 3852 | CA | PHE | D | 127 | −8.935 | 39.475 | 78.522 | 1.00 | 49.42 | D |
| ATOM | 3853 | CB | PHE | D | 127 | −8.758 | 40.972 | 78.272 | 1.00 | 47.62 | D |
| ATOM | 3854 | CG | PHE | D | 127 | −7.797 | 41.296 | 77.159 | 1.00 | 50.03 | D |
| ATOM | 3855 | CD1 | PHE | D | 127 | −8.034 | 40.845 | 75.860 | 1.00 | 44.74 | D |
| ATOM | 3856 | CD2 | PHE | D | 127 | −6.674 | 42.090 | 77.403 | 1.00 | 48.47 | D |
| ATOM | 3857 | CE1 | PHE | D | 127 | −7.180 | 41.180 | 74.819 | 1.00 | 47.40 | D |
| ATOM | 3858 | CE2 | PHE | D | 127 | −5.806 | 42.434 | 76.365 | 1.00 | 51.43 | D |
| ATOM | 3859 | CZ | PHE | D | 127 | −6.062 | 41.978 | 75.066 | 1.00 | 50.00 | D |
| ATOM | 3860 | C | PHE | D | 127 | −7.584 | 38.836 | 78.866 | 1.00 | 52.80 | D |
| ATOM | 3861 | O | PHE | D | 127 | −7.015 | 39.091 | 79.927 | 1.00 | 51.97 | D |
| ATOM | 3862 | N | ASP | D | 128 | −7.070 | 38.001 | 77.972 | 1.00 | 54.93 | D |
| ATOM | 3863 | CA | ASP | D | 128 | −5.800 | 37.329 | 78.223 | 1.00 | 55.92 | D |
| ATOM | 3864 | CB | ASP | D | 128 | −5.896 | 35.870 | 77.763 | 1.00 | 57.37 | D |
| ATOM | 3865 | CG | ASP | D | 128 | −4.713 | 35.035 | 78.208 | 1.00 | 59.20 | D |
| ATOM | 3866 | OD1 | ASP | D | 128 | −3.626 | 35.608 | 78.445 | 1.00 | 63.00 | D |
| ATOM | 3867 | OD2 | ASP | D | 128 | −4.862 | 33.800 | 78.305 | 1.00 | 59.97 | D |
| ATOM | 3868 | C | ASP | D | 128 | −4.657 | 38.054 | 77.495 | 1.00 | 57.15 | D |
| ATOM | 3869 | O | ASP | D | 128 | −4.271 | 37.682 | 76.379 | 1.00 | 53.80 | D |
| ATOM | 3870 | N | GLU | D | 129 | −4.124 | 39.085 | 78.150 | 1.00 | 59.48 | D |
| ATOM | 3871 | CA | GLU | D | 129 | −3.041 | 39.909 | 77.609 | 1.00 | 62.03 | D |
| ATOM | 3872 | CB | GLU | D | 129 | −2.575 | 40.919 | 78.651 | 1.00 | 67.03 | D |
| ATOM | 3873 | CG | GLU | D | 129 | −3.370 | 42.203 | 78.686 | 1.00 | 74.59 | D |
| ATOM | 3874 | CD | GLU | D | 129 | −2.796 | 43.184 | 79.685 | 1.00 | 78.87 | D |
| ATOM | 3875 | OE1 | GLU | D | 129 | −1.630 | 43.602 | 79.506 | 1.00 | 80.16 | D |
| ATOM | 3876 | OE2 | GLU | D | 129 | −3.505 | 43.527 | 80.656 | 1.00 | 81.80 | D |
| ATOM | 3877 | C | GLU | D | 129 | −1.816 | 39.178 | 77.083 | 1.00 | 60.28 | D |
| ATOM | 3878 | O | GLU | D | 129 | −1.364 | 39.443 | 75.963 | 1.00 | 59.39 | D |
| ATOM | 3879 | N | GLU | D | 130 | −1.257 | 38.283 | 77.892 | 1.00 | 58.70 | D |
| ATOM | 3880 | CA | GLU | D | 130 | −0.077 | 37.548 | 77.460 | 1.00 | 61.09 | D |
| ATOM | 3881 | CB | GLU | D | 130 | 0.381 | 36.568 | 78.551 | 1.00 | 63.54 | D |
| ATOM | 3882 | CG | GLU | D | 130 | 1.636 | 35.778 | 78.186 | 1.00 | 67.94 | D |
| ATOM | 3883 | CD | GLU | D | 130 | 2.201 | 34.972 | 79.349 | 1.00 | 72.22 | D |
| ATOM | 3884 | OE1 | GLU | D | 130 | 1.447 | 34.169 | 79.949 | 1.00 | 72.20 | D |
| ATOM | 3885 | OE2 | GLU | D | 130 | 3.406 | 35.138 | 79.656 | 1.00 | 71.99 | D |
| ATOM | 3886 | C | GLU | D | 130 | −0.346 | 36.807 | 76.142 | 1.00 | 61.20 | D |
| ATOM | 3887 | O | GLU | D | 130 | 0.350 | 37.037 | 75.150 | 1.00 | 61.12 | D |
| ATOM | 3888 | N | ARG | D | 131 | −1.361 | 35.941 | 76.121 | 1.00 | 60.76 | D |
| ATOM | 3889 | CA | ARG | D | 131 | −1.682 | 35.194 | 74.907 | 1.00 | 59.96 | D |
| ATOM | 3890 | CB | ARG | D | 131 | −2.739 | 34.111 | 75.183 | 1.00 | 60.14 | D |
| ATOM | 3891 | CG | ARG | D | 131 | −2.357 | 33.154 | 76.315 | 1.00 | 65.06 | D |
| ATOM | 3892 | CD | ARG | D | 131 | −3.070 | 31.796 | 76.249 | 1.00 | 67.01 | D |
| ATOM | 3893 | NE | ARG | D | 131 | −2.799 | 30.982 | 77.442 | 1.00 | 67.59 | D |
| ATOM | 3894 | CZ | ARG | D | 131 | −3.015 | 29.669 | 77.534 | 1.00 | 68.47 | D |
| ATOM | 3895 | NH1 | ARG | D | 131 | −3.505 | 28.991 | 76.502 | 1.00 | 67.19 | D |
| ATOM | 3896 | NH2 | ARG | D | 131 | −2.746 | 29.030 | 78.665 | 1.00 | 68.04 | D |
| ATOM | 3897 | C | ARG | D | 131 | −2.164 | 36.124 | 73.804 | 1.00 | 58.66 | D |
| ATOM | 3898 | O | ARG | D | 131 | −2.002 | 35.833 | 72.626 | 1.00 | 59.38 | D |
| ATOM | 3899 | N | ALA | D | 132 | −2.747 | 37.252 | 74.180 | 1.00 | 57.28 | D |
| ATOM | 3900 | CA | ALA | D | 132 | −3.232 | 38.185 | 73.177 | 1.00 | 59.68 | D |
| ATOM | 3901 | CB | ALA | D | 132 | −4.128 | 39.235 | 73.816 | 1.00 | 58.96 | D |
| ATOM | 3902 | C | ALA | D | 132 | −2.061 | 38.853 | 72.497 | 1.00 | 61.96 | D |
| ATOM | 3903 | O | ALA | D | 132 | −2.163 | 39.269 | 71.345 | 1.00 | 62.19 | D |
| ATOM | 3904 | N | GLN | D | 133 | −0.948 | 38.953 | 73.220 | 1.00 | 64.84 | D |
| ATOM | 3905 | CA | GLN | D | 133 | 0.253 | 39.591 | 72.695 | 1.00 | 66.77 | D |
| ATOM | 3906 | CB | GLN | D | 133 | 1.014 | 40.289 | 73.822 | 1.00 | 68.20 | D |
| ATOM | 3907 | CG | GLN | D | 133 | 0.352 | 41.568 | 74.304 | 1.00 | 68.90 | D |
| ATOM | 3908 | CD | GLN | D | 133 | 1.236 | 42.371 | 75.240 | 1.00 | 68.95 | D |
| ATOM | 3909 | OE1 | GLN | D | 133 | 0.892 | 43.489 | 75.626 | 1.00 | 68.24 | D |
| ATOM | 3910 | NE2 | GLN | D | 133 | 2.382 | 41.803 | 75.613 | 1.00 | 69.63 | D |
| ATOM | 3911 | C | GLN | D | 133 | 1.194 | 38.659 | 71.949 | 1.00 | 67.12 | D |
| ATOM | 3912 | O | GLN | D | 133 | 2.055 | 39.116 | 71.199 | 1.00 | 67.73 | D |
| ATOM | 3913 | N | GLN | D | 134 | 1.038 | 37.358 | 72.155 | 1.00 | 67.74 | D |
| ATOM | 3914 | CA | GLN | D | 134 | 1.878 | 36.381 | 71.478 | 1.00 | 69.36 | D |
| ATOM | 3915 | CB | GLN | D | 134 | 1.939 | 35.088 | 72.291 | 1.00 | 70.36 | D |
| ATOM | 3916 | CG | GLN | D | 134 | 2.494 | 35.272 | 73.694 | 1.00 | 74.36 | D |
| ATOM | 3917 | CD | GLN | D | 134 | 2.446 | 33.995 | 74.517 | 1.00 | 76.77 | D |
| ATOM | 3918 | OE1 | GLN | D | 134 | 1.374 | 33.439 | 74.760 | 1.00 | 78.04 | D |
| ATOM | 3919 | NE2 | GLN | D | 134 | 3.610 | 33.524 | 74.951 | 1.00 | 76.78 | D |
| ATOM | 3920 | C | GLN | D | 134 | 1.317 | 36.100 | 70.084 | 1.00 | 71.20 | D |
| ATOM | 3921 | O | GLN | D | 134 | 1.929 | 35.392 | 69.285 | 1.00 | 71.01 | D |
| ATOM | 3922 | N | GLU | D | 135 | 0.146 | 36.664 | 69.799 | 1.00 | 72.73 | D |
| ATOM | 3923 | CA | GLU | D | 135 | −0.499 | 36.480 | 68.503 | 1.00 | 74.02 | D |
| ATOM | 3924 | CB | GLU | D | 135 | −2.004 | 36.753 | 68.605 | 1.00 | 72.83 | D |
| ATOM | 3925 | CG | GLU | D | 135 | −2.807 | 35.698 | 69.359 | 1.00 | 71.50 | D |
| ATOM | 3926 | CD | GLU | D | 135 | −2.831 | 34.355 | 68.655 | 1.00 | 70.06 | D |
| ATOM | 3927 | OE1 | GLU | D | 135 | −2.896 | 34.335 | 67.404 | 1.00 | 66.72 | D |
| ATOM | 3928 | OE2 | GLU | D | 135 | −2.802 | 33.321 | 69.356 | 1.00 | 69.82 | D |
| ATOM | 3929 | C | GLU | D | 135 | 0.112 | 37.418 | 67.473 | 1.00 | 75.50 | D |
| ATOM | 3930 | OT1 | GLU | D | 135 | −0.652 | 38.189 | 66.855 | 1.00 | 76.43 | D |
| ATOM | 3931 | OT2 | GLU | D | 135 | 1.348 | 37.366 | 67.296 | 1.00 | 77.20 | D |
| END | | | | | | | | | | | |

TABLE 4
REMARK coordinates from restrained individual B-factor refinement
REMARK refinement resolution: 500.0-2.6 A
REMARK starting r = 0.2391 free_r = 0.2748
REMARK final r = 0.2373 free_r = 0.2731
REMARK B rmsd for bonded mainchain atoms = 1.189 target = 1.5
REMARK B rmsd for bonded sidechain atoms = 1.805 target = 2.0
REMARK B rmsd for angle mainchain atoms = 2.070 target = 2.0
REMARK B rmsd for angle sidechain atoms = 2.761 target = 2.5
REMARK rweight = 0.0204 (with wa = 0.661678)
REMARK target = mlf steps = 30
REMARK sg = P4(3)2(1)2 a = 115.0331 b = 115.0331 c = 133.9252 alpha = 90 beta = 90
gamma = 90
REMARK parameter file 1: CNS_TOPPAR: protein_rep.param
REMARK parameter file 2: CNS_TOPPAR: dna-rna_rep.param
REMARK parameter file 3: CNS_TOPPAR: water_rep.param
REMARK molecular structure file: aml_07Dec00_g.mtf
REMARK input coordinates: aml_07Dec00_m.pdb
REMARK reflection file = . . . /c36_p43212.cv
REMARK ncs = restrain ncs file = ncs.def
REMARK B-correction resolution: 6.0-2.6
REMARK initial B-factor correction applied to fobs:
REMARK B11 = −8.893 B22 = −8.893 B33 = 17.786
REMARK B12 = 0.000 B13 = 0.000 B23 = 0.000
REMARK B-factor correction applied to coordinate array B: −0.655
REMARK bulk solvent: density level = 0.380253 e/A\hat{ }3, B-factor = 44.2281 A\hat{ }2
REMARK reflections with |Fobs|/sigma_F < 0.0 rejected
REMARK reflections with |Fobs| > 10000 * rms(Fobs) rejected
REMARK theoretical total number of refl. in resol. range: 28287 (100.0%)
REMARK number of unobserved reflections (no entry or |F| =0): 361 (1.3%)
REMARK number of reflections rejected: 0 (0.0%)
REMARK total number of reflections used: 27926 (98.7%)
REMARK number of reflections in working set: 25654 (90.7%)
REMARK number of reflections in test set: 2272 (8.0%)
CRYST1 115.033 115.033 133.925 90.00 90.00 90.00 P 43 21 2
REMARK
FILENAME = “/rlwgrp2/jero/ajw/esrf_oct00/c36/cns/a2h/aml_07Dec00_b.pdb”
REMARK DATE: 7-Dec-00 18:18:51 created by user: root
REMARK VERSION: 1.0

| | | | | | | | | | | | |
|---|---|---|---|---|---|---|---|---|---|---|---|
| ATOM | 1 | CB | ALA | A | 54 | 121.805 | 154.471 | 72.415 | 0.00 | 78.59 | A |
| ATOM | 2 | C | ALA | A | 54 | 123.574 | 154.993 | 70.716 | 0.00 | 78.96 | A |
| ATOM | 3 | O | ALA | A | 54 | 124.475 | 155.692 | 70.237 | 0.00 | 78.86 | A |
| ATOM | 4 | N | ALA | A | 54 | 122.228 | 156.807 | 71.753 | 0.00 | 78.62 | A |
| ATOM | 5 | CA | ALA | A | 54 | 122.861 | 155.474 | 71.990 | 0.00 | 78.77 | A |
| ATOM | 6 | N | ALA | A | 55 | 123.135 | 153.848 | 70.136 | 0.00 | 79.49 | A |
| ATOM | 7 | CA | ALA | A | 55 | 123.766 | 153.298 | 68.917 | 0.00 | 79.92 | A |
| ATOM | 8 | CB | ALA | A | 55 | 123.970 | 151.778 | 69.045 | 0.00 | 79.93 | A |
| ATOM | 9 | C | ALA | A | 55 | 123.031 | 153.604 | 67.608 | 0.00 | 80.20 | A |
| ATOM | 10 | O | ALA | A | 55 | 123.648 | 154.073 | 66.651 | 0.00 | 80.27 | A |
| ATOM | 11 | N | ALA | A | 56 | 121.727 | 153.328 | 67.565 | 0.00 | 80.48 | A |
| ATOM | 12 | CA | ALA | A | 56 | 120.904 | 153.576 | 66.371 | 0.00 | 80.61 | A |
| ATOM | 13 | CB | ALA | A | 56 | 121.021 | 155.060 | 65.968 | 0.00 | 80.72 | A |
| ATOM | 14 | C | ALA | A | 56 | 121.309 | 152.663 | 65.191 | 0.00 | 80.49 | A |
| ATOM | 15 | O | ALA | A | 56 | 122.437 | 152.787 | 64.705 | 0.00 | 80.77 | A |
| ATOM | 16 | N | ALA | A | 57 | 120.399 | 151.779 | 64.724 | 1.00 | 80.39 | A |
| ATOM | 17 | CA | ALA | A | 57 | 120.694 | 150.833 | 63.613 | 1.00 | 78.86 | A |
| ATOM | 18 | CB | ALA | A | 57 | 122.209 | 150.553 | 63.594 | 1.00 | 79.73 | A |
| ATOM | 19 | C | ALA | A | 57 | 119.970 | 149.454 | 63.591 | 1.00 | 77.96 | A |
| ATOM | 20 | O | ALA | A | 57 | 120.570 | 148.455 | 64.055 | 1.00 | 78.58 | A |
| ATOM | 21 | N | HIS | A | 58 | 118.761 | 149.333 | 63.019 | 1.00 | 75.64 | A |
| ATOM | 22 | CA | HIS | A | 58 | 118.098 | 148.004 | 63.047 | 1.00 | 72.57 | A |
| ATOM | 23 | CB | HIS | A | 58 | 117.350 | 147.842 | 64.400 | 1.00 | 75.27 | A |
| ATOM | 24 | CG | HIS | A | 58 | 115.878 | 148.185 | 64.347 | 1.00 | 78.58 | A |
| ATOM | 25 | CD2 | HIS | A | 58 | 115.159 | 149.138 | 64.996 | 1.00 | 79.75 | A |
| ATOM | 26 | ND1 | HIS | A | 58 | 114.966 | 147.460 | 63.594 | 1.00 | 79.33 | A |
| ATOM | 27 | CE1 | HIS | A | 58 | 113.751 | 147.948 | 63.784 | 1.00 | 80.34 | A |
| ATOM | 28 | NE2 | HIS | A | 58 | 113.837 | 148.964 | 64.631 | 1.00 | 81.89 | A |
| ATOM | 29 | C | HIS | A | 58 | 117.160 | 147.486 | 61.933 | 1.00 | 68.30 | A |
| ATOM | 30 | O | HIS | A | 58 | 116.957 | 146.273 | 61.833 | 1.00 | 68.88 | A |
| ATOM | 31 | N | PRO | A | 59 | 116.558 | 148.370 | 61.109 | 1.00 | 63.81 | A |
| ATOM | 32 | CD | PRO | A | 59 | 116.612 | 149.836 | 61.121 | 1.00 | 62.32 | A |
| ATOM | 33 | CA | PRO | A | 59 | 115.640 | 147.929 | 60.052 | 1.00 | 60.08 | A |
| ATOM | 34 | CB | PRO | A | 59 | 115.320 | 149.219 | 59.315 | 1.00 | 59.30 | A |
| ATOM | 35 | CG | PRO | A | 59 | 115.374 | 150.189 | 60.344 | 1.00 | 60.57 | A |
| ATOM | 36 | C | PRO | A | 59 | 116.150 | 146.864 | 59.111 | 1.00 | 57.19 | A |
| ATOM | 37 | O | PRO | A | 59 | 115.391 | 146.037 | 58.632 | 1.00 | 55.71 | A |
| ATOM | 38 | N | GLY | A | 60 | 117.448 | 146.925 | 58.828 | 1.00 | 55.51 | A |
| ATOM | 39 | CA | GLY | A | 60 | 118.081 | 145.989 | 57.925 | 1.00 | 52.11 | A |
| ATOM | 40 | C | GLY | A | 60 | 117.934 | 146.284 | 56.438 | 1.00 | 50.92 | A |
| ATOM | 41 | O | GLY | A | 60 | 117.859 | 147.423 | 56.007 | 1.00 | 51.10 | A |
| ATOM | 42 | N | GLU | A | 61 | 117.873 | 145.224 | 55.655 | 1.00 | 48.71 | A |
| ATOM | 43 | CA | GLU | A | 61 | 117.772 | 145.293 | 54.217 | 1.00 | 47.54 | A |
| ATOM | 44 | CB | GLU | A | 61 | 118.354 | 144.018 | 53.705 | 1.00 | 50.06 | A |
| ATOM | 45 | CG | GLU | A | 61 | 118.083 | 143.821 | 52.290 | 1.00 | 56.16 | A |
| ATOM | 46 | CD | GLU | A | 61 | 119.305 | 144.018 | 51.479 | 1.00 | 58.82 | A |
| ATOM | 47 | OE1 | GLU | A | 61 | 119.703 | 145.204 | 51.301 | 1.00 | 60.71 | A |
| ATOM | 48 | OE2 | GLU | A | 61 | 119.859 | 142.967 | 51.053 | 1.00 | 59.21 | A |
| ATOM | 49 | C | GLU | A | 61 | 116.325 | 145.460 | 53.722 | 1.00 | 45.18 | A |
| ATOM | 50 | O | GLU | A | 61 | 115.556 | 144.500 | 53.727 | 1.00 | 45.45 | A |
| ATOM | 51 | N | LEU | A | 62 | 115.955 | 146.652 | 53.263 | 1.00 | 41.60 | A |
| ATOM | 52 | CA | LEU | A | 62 | 114.572 | 146.905 | 52.870 | 1.00 | 37.79 | A |
| ATOM | 53 | CB | LEU | A | 62 | 114.031 | 148.127 | 53.640 | 1.00 | 37.37 | A |
| ATOM | 54 | CG | LEU | A | 62 | 114.196 | 148.167 | 55.174 | 1.00 | 37.06 | A |
| ATOM | 55 | CD1 | LEU | A | 62 | 113.745 | 149.536 | 55.792 | 1.00 | 36.59 | A |
| ATOM | 56 | CD2 | LEU | A | 62 | 113.401 | 147.012 | 55.756 | 1.00 | 36.81 | A |
| ATOM | 57 | C | LEU | A | 62 | 114.351 | 147.147 | 51.426 | 1.00 | 35.89 | A |
| ATOM | 58 | O | LEU | A | 62 | 115.247 | 147.604 | 50.725 | 1.00 | 35.12 | A |
| ATOM | 59 | N | VAL | A | 63 | 113.131 | 146.869 | 50.994 | 1.00 | 33.87 | A |
| ATOM | 60 | CA | VAL | A | 63 | 112.739 | 147.117 | 49.613 | 1.00 | 33.61 | A |
| ATOM | 61 | CB | VAL | A | 63 | 112.648 | 145.854 | 48.767 | 1.00 | 32.78 | A |
| ATOM | 62 | CG1 | VAL | A | 63 | 113.981 | 145.266 | 48.678 | 1.00 | 32.46 | A |
| ATOM | 63 | CG2 | VAL | A | 63 | 111.705 | 144.868 | 49.349 | 1.00 | 32.47 | A |
| ATOM | 64 | C | VAL | A | 63 | 111.406 | 147.755 | 49.618 | 1.00 | 33.83 | A |
| ATOM | 65 | O | VAL | A | 63 | 110.694 | 147.686 | 50.600 | 1.00 | 34.75 | A |
| ATOM | 66 | N | ARG | A | 64 | 111.044 | 148.333 | 48.495 | 1.00 | 34.54 | A |
| ATOM | 67 | CA | ARG | A | 64 | 109.774 | 149.027 | 48.396 | 1.00 | 34.70 | A |
| ATOM | 68 | CB | ARG | A | 64 | 109.746 | 149.945 | 47.184 | 1.00 | 33.85 | A |
| ATOM | 69 | CG | ARG | A | 64 | 110.624 | 151.100 | 47.304 | 1.00 | 35.64 | A |
| ATOM | 70 | CD | ARG | A | 64 | 110.364 | 151.912 | 46.097 | 1.00 | 39.94 | A |
| ATOM | 71 | NE | ARG | A | 64 | 111.164 | 153.135 | 46.028 | 1.00 | 44.04 | A |
| ATOM | 72 | CZ | ARG | A | 64 | 111.071 | 154.166 | 46.871 | 1.00 | 44.33 | A |
| ATOM | 73 | NH1 | ARG | A | 64 | 110.204 | 154.156 | 47.895 | 1.00 | 42.71 | A |
| ATOM | 74 | NH2 | ARG | A | 64 | 111.866 | 155.208 | 46.666 | 1.00 | 45.29 | A |
| ATOM | 75 | C | ARG | A | 64 | 108.676 | 148.074 | 48.254 | 1.00 | 33.13 | A |
| ATOM | 76 | O | ARG | A | 64 | 108.869 | 147.021 | 47.750 | 1.00 | 35.16 | A |
| ATOM | 77 | N | THR | A | 65 | 107.519 | 148.429 | 48.725 | 1.00 | 32.07 | A |
| ATOM | 78 | CA | THR | A | 65 | 106.417 | 147.541 | 48.505 | 1.00 | 30.87 | A |
| ATOM | 79 | CB | THR | A | 65 | 105.482 | 147.431 | 49.722 | 1.00 | 29.09 | A |
| ATOM | 80 | OG1 | THR | A | 65 | 104.748 | 148.655 | 49.857 | 1.00 | 27.91 | A |
| ATOM | 81 | CG2 | THR | A | 65 | 106.263 | 147.155 | 50.970 | 1.00 | 27.11 | A |
| ATOM | 82 | C | THR | A | 65 | 105.672 | 148.281 | 47.411 | 1.00 | 31.70 | A |
| ATOM | 83 | O | THR | A | 65 | 106.122 | 149.333 | 46.973 | 1.00 | 32.17 | A |
| ATOM | 84 | N | ASP | A | 66 | 104.530 | 147.754 | 46.985 | 1.00 | 31.73 | A |
| ATOM | 85 | CA | ASP | A | 66 | 103.721 | 148.421 | 45.980 | 1.00 | 31.66 | A |
| ATOM | 86 | CB | ASP | A | 66 | 102.661 | 147.498 | 45.368 | 1.00 | 32.40 | A |
| ATOM | 87 | CG | ASP | A | 66 | 103.256 | 146.386 | 44.568 | 1.00 | 35.11 | A |
| ATOM | 88 | OD1 | ASP | A | 66 | 104.390 | 146.578 | 44.057 | 1.00 | 38.19 | A |
| ATOM | 89 | OD2 | ASP | A | 66 | 102.607 | 145.324 | 44.451 | 1.00 | 34.50 | A |
| ATOM | 90 | C | ASP | A | 66 | 102.970 | 149.585 | 46.611 | 1.00 | 32.17 | A |
| ATOM | 91 | O | ASP | A | 66 | 102.141 | 150.216 | 45.940 | 1.00 | 34.33 | A |
| ATOM | 92 | N | SER | A | 67 | 103.209 | 149.869 | 47.884 | 1.00 | 30.67 | A |
| ATOM | 93 | CA | SER | A | 67 | 102.534 | 150.986 | 48.492 | 1.00 | 30.41 | A |
| ATOM | 94 | CB | SER | A | 67 | 101.845 | 150.582 | 49.797 | 1.00 | 30.65 | A |
| ATOM | 95 | OG | SER | A | 67 | 101.630 | 151.725 | 50.615 | 1.00 | 29.34 | A |
| ATOM | 96 | C | SER | A | 67 | 103.555 | 152.071 | 48.779 | 1.00 | 30.34 | A |
| ATOM | 97 | O | SER | A | 67 | 104.658 | 151.793 | 49.164 | 1.00 | 32.01 | A |
| ATOM | 98 | N | PRO | A | 68 | 103.183 | 153.318 | 48.590 | 1.00 | 29.68 | A |
| ATOM | 99 | CD | PRO | A | 68 | 101.884 | 153.705 | 47.990 | 1.00 | 30.07 | A |
| ATOM | 100 | CA | PRO | A | 68 | 104.060 | 154.463 | 48.835 | 1.00 | 29.32 | A |
| ATOM | 101 | CB | PRO | A | 68 | 103.344 | 155.608 | 48.116 | 1.00 | 29.75 | A |
| ATOM | 102 | CG | PRO | A | 68 | 101.842 | 155.192 | 48.229 | 1.00 | 31.39 | A |
| ATOM | 103 | C | PRO | A | 68 | 104.221 | 154.767 | 50.316 | 1.00 | 29.03 | A |
| ATOM | 104 | O | PRO | A | 68 | 104.914 | 155.702 | 50.697 | 1.00 | 29.56 | A |
| ATOM | 105 | N | ASN | A | 69 | 103.573 | 153.975 | 51.156 | 1.00 | 29.41 | A |
| ATOM | 106 | CA | ASN | A | 69 | 103.635 | 154.206 | 52.601 | 1.00 | 28.86 | A |
| ATOM | 107 | CB | ASN | A | 69 | 102.225 | 154.410 | 53.209 | 1.00 | 28.48 | A |
| ATOM | 108 | CG | ASN | A | 69 | 102.263 | 155.394 | 54.344 | 1.00 | 31.64 | A |
| ATOM | 109 | OD1 | ASN | A | 69 | 103.195 | 156.199 | 54.389 | 1.00 | 35.05 | A |
| ATOM | 110 | ND2 | ASN | A | 69 | 101.292 | 155.367 | 55.262 | 1.00 | 30.14 | A |
| ATOM | 111 | C | ASN | A | 69 | 104.337 | 153.099 | 53.358 | 1.00 | 28.44 | A |
| ATOM | 112 | O | ASN | A | 69 | 104.351 | 153.103 | 54.584 | 1.00 | 28.34 | A |
| ATOM | 113 | N | PHE | A | 70 | 104.913 | 152.132 | 52.650 | 1.00 | 28.40 | A |
| ATOM | 114 | CA | PHE | A | 70 | 105.570 | 151.041 | 53.374 | 1.00 | 28.37 | A |
| ATOM | 115 | CB | PHE | A | 70 | 104.602 | 149.885 | 53.680 | 1.00 | 27.32 | A |
| ATOM | 116 | CG | PHE | A | 70 | 103.391 | 150.291 | 54.428 | 1.00 | 26.92 | A |
| ATOM | 117 | CD1 | PHE | A | 70 | 102.237 | 150.663 | 53.764 | 1.00 | 24.04 | A |
| ATOM | 118 | CD2 | PHE | A | 70 | 103.438 | 150.413 | 55.821 | 1.00 | 26.89 | A |
| ATOM | 119 | CE1 | PHE | A | 70 | 101.144 | 151.174 | 54.490 | 1.00 | 26.56 | A |
| ATOM | 120 | CE2 | PHE | A | 70 | 102.334 | 150.926 | 56.535 | 1.00 | 27.80 | A |
| ATOM | 121 | CZ | PHE | A | 70 | 101.189 | 151.313 | 55.875 | 1.00 | 25.26 | A |
| ATOM | 122 | C | PHE | A | 70 | 106.716 | 150.405 | 52.642 | 1.00 | 29.14 | A |
| ATOM | 123 | O | PHE | A | 70 | 106.705 | 150.274 | 51.435 | 1.00 | 30.85 | A |
| ATOM | 124 | N | LEU | A | 71 | 107.701 | 149.987 | 53.404 | 1.00 | 29.58 | A |
| ATOM | 125 | CA | LEU | A | 71 | 108.826 | 149.274 | 52.870 | 1.00 | 28.89 | A |
| ATOM | 126 | CB | LEU | A | 71 | 110.106 | 150.004 | 53.240 | 1.00 | 26.89 | A |
| ATOM | 127 | CG | LEU | A | 71 | 110.340 | 151.432 | 52.803 | 1.00 | 28.10 | A |
| ATOM | 128 | CD1 | LEU | A | 71 | 111.798 | 151.833 | 53.194 | 1.00 | 25.31 | A |
| ATOM | 129 | CD2 | LEU | A | 71 | 110.097 | 151.591 | 51.279 | 1.00 | 25.66 | A |
| ATOM | 130 | C | LEU | A | 71 | 108.782 | 147.891 | 53.611 | 1.00 | 29.76 | A |
| ATOM | 131 | O | LEU | A | 71 | 108.124 | 147.726 | 54.649 | 1.00 | 30.51 | A |
| ATOM | 132 | N | CYS | A | 72 | 109.420 | 146.872 | 53.077 | 1.00 | 29.19 | A |
| ATOM | 133 | CA | CYS | A | 72 | 109.492 | 145.660 | 53.881 | 1.00 | 31.23 | A |
| ATOM | 134 | CB | CYS | A | 72 | 108.356 | 144.696 | 53.546 | 1.00 | 30.42 | A |
| ATOM | 135 | SG | CYS | A | 72 | 108.556 | 143.871 | 51.981 | 1.00 | 33.44 | A |
| ATOM | 136 | C | CYS | A | 72 | 110.887 | 145.000 | 53.695 | 1.00 | 31.06 | A |
| ATOM | 137 | O | CYS | A | 72 | 111.641 | 145.382 | 52.813 | 1.00 | 31.82 | A |
| ATOM | 138 | N | SER | A | 73 | 111.240 | 144.046 | 54.536 | 1.00 | 31.97 | A |
| ATOM | 139 | CA | SER | A | 73 | 112.534 | 143.382 | 54.416 | 1.00 | 33.13 | A |
| ATOM | 140 | CB | SER | A | 73 | 112.746 | 142.333 | 55.509 | 1.00 | 32.76 | A |
| ATOM | 141 | OG | SER | A | 73 | 112.335 | 142.831 | 56.752 | 1.00 | 37.23 | A |
| ATOM | 142 | C | SER | A | 73 | 112.585 | 142.636 | 53.099 | 1.00 | 33.33 | A |
| ATOM | 143 | O | SER | A | 73 | 111.555 | 142.310 | 52.509 | 1.00 | 33.85 | A |
| ATOM | 144 | N | VAL | A | 74 | 113.800 | 142.354 | 52.677 | 1.00 | 32.21 | A |
| ATOM | 145 | CA | VAL | A | 74 | 114.059 | 141.611 | 51.496 | 1.00 | 33.13 | A |
| ATOM | 146 | CB | VAL | A | 74 | 115.512 | 141.909 | 51.007 | 1.00 | 35.49 | A |
| ATOM | 147 | CG1 | VAL | A | 74 | 115.927 | 140.942 | 49.948 | 1.00 | 35.23 | A |
| ATOM | 148 | CG2 | VAL | A | 74 | 115.563 | 143.332 | 50.460 | 1.00 | 35.82 | A |
| ATOM | 149 | C | VAL | A | 74 | 113.927 | 140.146 | 51.887 | 1.00 | 31.58 | A |
| ATOM | 150 | O | VAL | A | 74 | 114.449 | 139.732 | 52.893 | 1.00 | 31.89 | A |
| ATOM | 151 | N | LEU | A | 75 | 113.233 | 139.372 | 51.080 | 1.00 | 30.67 | A |
| ATOM | 152 | CA | LEU | A | 75 | 113.041 | 137.959 | 51.342 | 1.00 | 29.90 | A |
| ATOM | 153 | CB | LEU | A | 75 | 111.592 | 137.606 | 51.102 | 1.00 | 28.35 | A |
| ATOM | 154 | CG | LEU | A | 75 | 110.616 | 138.076 | 52.156 | 1.00 | 27.35 | A |
| ATOM | 155 | CD1 | LEU | A | 75 | 109.181 | 137.922 | 51.659 | 1.00 | 25.52 | A |
| ATOM | 156 | CD2 | LEU | A | 75 | 110.899 | 137.241 | 53.437 | 1.00 | 25.42 | A |
| ATOM | 157 | C | LEU | A | 75 | 113.880 | 137.109 | 50.392 | 1.00 | 31.81 | A |
| ATOM | 158 | O | LEU | A | 75 | 114.266 | 137.542 | 49.304 | 1.00 | 32.66 | A |
| ATOM | 159 | N | PRO | A | 76 | 114.168 | 135.877 | 50.785 | 1.00 | 32.77 | A |
| ATOM | 160 | CD | PRO | A | 76 | 113.675 | 135.168 | 51.985 | 1.00 | 32.79 | A |
| ATOM | 161 | CA | PRO | A | 76 | 114.957 | 135.013 | 49.892 | 1.00 | 31.76 | A |
| ATOM | 162 | CB | PRO | A | 76 | 115.096 | 133.709 | 50.659 | 1.00 | 31.25 | A |
| ATOM | 163 | CG | PRO | A | 76 | 114.665 | 134.036 | 52.081 | 1.00 | 33.58 | A |
| ATOM | 164 | C | PRO | A | 76 | 114.085 | 134.796 | 48.681 | 1.00 | 32.51 | A |
| ATOM | 165 | O | PRO | A | 76 | 112.860 | 134.795 | 48.789 | 1.00 | 33.70 | A |
| ATOM | 166 | N | THR | A | 77 | 114.696 | 134.593 | 47.529 | 1.00 | 33.67 | A |
| ATOM | 167 | CA | THR | A | 77 | 113.922 | 134.367 | 46.303 | 1.00 | 34.58 | A |
| ATOM | 168 | CB | THR | A | 77 | 114.867 | 134.304 | 45.072 | 1.00 | 36.17 | A |
| ATOM | 169 | OG1 | THR | A | 77 | 115.252 | 135.627 | 44.710 | 1.00 | 39.47 | A |
| ATOM | 170 | CG2 | THR | A | 77 | 114.156 | 133.718 | 43.865 | 1.00 | 38.34 | A |
| ATOM | 171 | C | THR | A | 77 | 113.141 | 133.067 | 46.333 | 1.00 | 34.54 | A |
| ATOM | 172 | O | THR | A | 77 | 112.015 | 132.977 | 45.808 | 1.00 | 35.01 | A |
| ATOM | 173 | N | HIS | A | 78 | 113.794 | 132.058 | 46.898 | 1.00 | 32.92 | A |
| ATOM | 174 | CA | HIS | A | 78 | 113.281 | 130.716 | 46.997 | 1.00 | 33.13 | A |
| ATOM | 175 | CB | HIS | A | 78 | 114.057 | 129.856 | 45.994 | 1.00 | 31.65 | A |
| ATOM | 176 | CG | HIS | A | 78 | 113.691 | 128.412 | 46.001 | 1.00 | 31.69 | A |
| ATOM | 177 | CD2 | HIS | A | 78 | 112.540 | 127.776 | 46.329 | 1.00 | 31.77 | A |
| ATOM | 178 | ND1 | HIS | A | 78 | 114.540 | 127.445 | 45.514 | 1.00 | 31.66 | A |
| ATOM | 179 | CE1 | HIS | A | 78 | 113.926 | 126.274 | 45.532 | 1.00 | 30.75 | A |
| ATOM | 180 | NE2 | HIS | A | 78 | 112.711 | 126.449 | 46.020 | 1.00 | 31.25 | A |
| ATOM | 181 | C | HIS | A | 78 | 113.573 | 130.252 | 48.445 | 1.00 | 33.65 | A |
| ATOM | 182 | O | HIS | A | 78 | 114.706 | 130.441 | 48.971 | 1.00 | 33.27 | A |
| ATOM | 183 | N | TRP | A | 79 | 112.562 | 129.665 | 49.091 | 1.00 | 31.97 | A |
| ATOM | 184 | CA | TRP | A | 79 | 112.728 | 129.167 | 50.456 | 1.00 | 30.51 | A |
| ATOM | 185 | CB | TRP | A | 79 | 112.351 | 130.214 | 51.495 | 1.00 | 28.72 | A |
| ATOM | 186 | CG | TRP | A | 79 | 112.994 | 129.985 | 52.805 | 1.00 | 28.24 | A |
| ATOM | 187 | CD2 | TRP | A | 79 | 114.397 | 130.061 | 53.082 | 1.00 | 27.36 | A |
| ATOM | 188 | CE2 | TRP | A | 79 | 114.563 | 129.845 | 54.478 | 1.00 | 27.95 | A |
| ATOM | 189 | CE3 | TRP | A | 79 | 115.538 | 130.292 | 52.286 | 1.00 | 27.33 | A |
| ATOM | 190 | CD1 | TRP | A | 79 | 112.372 | 129.712 | 54.016 | 1.00 | 27.47 | A |
| ATOM | 191 | NE1 | TRP | A | 79 | 113.311 | 129.634 | 55.013 | 1.00 | 28.93 | A |
| ATOM | 192 | CZ2 | TRP | A | 79 | 115.819 | 129.859 | 55.098 | 1.00 | 25.98 | A |
| ATOM | 193 | CZ3 | TRP | A | 79 | 116.802 | 130.310 | 52.908 | 1.00 | 25.58 | A |
| ATOM | 194 | CH2 | TRP | A | 79 | 116.918 | 130.095 | 54.311 | 1.00 | 25.92 | A |
| ATOM | 195 | C | TRP | A | 79 | 111.891 | 127.924 | 50.704 | 1.00 | 30.23 | A |
| ATOM | 196 | O | TRP | A | 79 | 110.926 | 127.621 | 49.985 | 1.00 | 30.01 | A |
| ATOM | 197 | N | ARG | A | 80 | 112.257 | 127.230 | 51.766 | 1.00 | 30.10 | A |
| ATOM | 198 | CA | ARG | A | 80 | 111.605 | 125.997 | 52.108 | 1.00 | 31.34 | A |
| ATOM | 199 | CB | ARG | A | 80 | 112.537 | 125.105 | 52.939 | 1.00 | 30.35 | A |
| ATOM | 200 | CG | ARG | A | 80 | 112.107 | 123.698 | 52.959 | 1.00 | 28.95 | A |
| ATOM | 201 | CD | ARG | A | 80 | 113.169 | 122.808 | 53.552 | 1.00 | 27.85 | A |
| ATOM | 202 | NE | ARG | A | 80 | 112.741 | 121.404 | 53.463 | 1.00 | 28.83 | A |
| ATOM | 203 | CZ | ARG | A | 80 | 113.461 | 120.383 | 53.929 | 1.00 | 31.77 | A |
| ATOM | 204 | NH1 | ARG | A | 80 | 114.663 | 120.600 | 54.540 | 1.00 | 32.07 | A |
| ATOM | 205 | NH2 | ARG | A | 80 | 113.015 | 119.129 | 53.745 | 1.00 | 31.19 | A |
| ATOM | 206 | C | ARG | A | 80 | 110.309 | 126.226 | 52.839 | 1.00 | 33.42 | A |
| ATOM | 207 | O | ARG | A | 80 | 110.153 | 127.110 | 53.698 | 1.00 | 34.45 | A |
| ATOM | 208 | N | CYS | A | 81 | 109.357 | 125.390 | 52.532 | 1.00 | 34.43 | A |
| ATOM | 209 | CA | CYS | A | 81 | 108.113 | 125.574 | 53.165 | 1.00 | 36.98 | A |
| ATOM | 210 | CB | CYS | A | 81 | 107.088 | 124.767 | 52.385 | 1.00 | 40.49 | A |
| ATOM | 211 | SG | CYS | A | 81 | 106.693 | 123.298 | 53.158 | 1.00 | 50.53 | A |
| ATOM | 212 | C | CYS | A | 81 | 108.247 | 125.199 | 54.657 | 1.00 | 36.69 | A |
| ATOM | 213 | O | CYS | A | 81 | 109.003 | 124.276 | 55.056 | 1.00 | 36.89 | A |
| ATOM | 214 | N | ASN | A | 82 | 107.534 | 125.956 | 55.485 | 1.00 | 35.37 | A |
| ATOM | 215 | CA | ASN | A | 82 | 107.550 | 125.794 | 56.935 | 1.00 | 33.84 | A |
| ATOM | 216 | CB | ASN | A | 82 | 107.017 | 124.438 | 57.380 | 1.00 | 34.42 | A |
| ATOM | 217 | CG | ASN | A | 82 | 106.859 | 124.364 | 58.904 | 1.00 | 36.39 | A |
| ATOM | 218 | OD1 | ASN | A | 82 | 106.305 | 125.272 | 59.549 | 1.00 | 37.70 | A |
| ATOM | 219 | ND2 | ASN | A | 82 | 107.340 | 123.292 | 59.484 | 1.00 | 35.85 | A |
| ATOM | 220 | C | ASN | A | 82 | 108.889 | 126.037 | 57.622 | 1.00 | 33.07 | A |
| ATOM | 221 | O | ASN | A | 82 | 109.039 | 125.782 | 58.799 | 1.00 | 33.33 | A |
| ATOM | 222 | N | LYS | A | 83 | 109.846 | 126.582 | 56.890 | 1.00 | 31.40 | A |
| ATOM | 223 | CA | LYS | A | 83 | 111.141 | 126.828 | 57.452 | 1.00 | 30.89 | A |
| ATOM | 224 | CB | LYS | A | 83 | 112.202 | 126.358 | 56.465 | 1.00 | 29.98 | A |
| ATOM | 225 | CG | LYS | A | 83 | 113.595 | 126.716 | 56.843 | 1.00 | 27.34 | A |
| ATOM | 226 | CD | LYS | A | 83 | 114.521 | 126.146 | 55.857 | 1.00 | 27.26 | A |
| ATOM | 227 | CE | LYS | A | 83 | 115.940 | 126.435 | 56.270 | 1.00 | 28.86 | A |
| ATOM | 228 | NZ | LYS | A | 83 | 116.931 | 125.774 | 55.395 | 1.00 | 30.72 | A |
| ATOM | 229 | C | LYS | A | 83 | 111.397 | 128.273 | 57.841 | 1.00 | 32.81 | A |
| ATOM | 230 | O | LYS | A | 83 | 111.104 | 129.200 | 57.099 | 1.00 | 35.29 | A |
| ATOM | 231 | N | THR | A | 84 | 111.956 | 128.467 | 59.015 | 1.00 | 33.57 | A |
| ATOM | 232 | CA | THR | A | 84 | 112.285 | 129.775 | 59.524 | 1.00 | 32.76 | A |
| ATOM | 233 | CB | THR | A | 84 | 113.120 | 129.603 | 60.855 | 1.00 | 34.21 | A |
| ATOM | 234 | OG1 | THR | A | 84 | 112.233 | 129.099 | 61.884 | 1.00 | 34.21 | A |
| ATOM | 235 | CG2 | THR | A | 84 | 113.807 | 130.911 | 61.287 | 1.00 | 30.27 | A |
| ATOM | 236 | C | THR | A | 84 | 113.102 | 130.508 | 58.495 | 1.00 | 33.07 | A |
| ATOM | 237 | O | THR | A | 84 | 114.050 | 129.964 | 57.933 | 1.00 | 34.35 | A |
| ATOM | 238 | N | LEU | A | 85 | 112.749 | 131.756 | 58.265 | 1.00 | 33.04 | A |
| ATOM | 239 | CA | LEU | A | 85 | 113.451 | 132.591 | 57.296 | 1.00 | 32.77 | A |
| ATOM | 240 | CB | LEU | A | 85 | 112.646 | 133.847 | 57.074 | 1.00 | 30.05 | A |
| ATOM | 241 | CG | LEU | A | 85 | 111.263 | 133.595 | 56.521 | 1.00 | 29.92 | A |
| ATOM | 242 | CD1 | LEU | A | 85 | 110.412 | 134.856 | 56.630 | 1.00 | 27.51 | A |
| ATOM | 243 | CD2 | LEU | A | 85 | 111.435 | 133.157 | 55.051 | 1.00 | 29.25 | A |
| ATOM | 244 | C | LEU | A | 85 | 114.854 | 132.991 | 57.718 | 1.00 | 33.82 | A |
| ATOM | 245 | O | LEU | A | 85 | 115.174 | 133.024 | 58.872 | 1.00 | 33.38 | A |
| ATOM | 246 | N | PRO | A | 86 | 115.707 | 133.342 | 56.762 | 1.00 | 36.50 | A |
| ATOM | 247 | CD | PRO | A | 86 | 115.523 | 133.259 | 55.295 | 1.00 | 36.99 | A |
| ATOM | 248 | CA | PRO | A | 86 | 117.084 | 133.750 | 57.120 | 1.00 | 37.45 | A |
| ATOM | 249 | CB | PRO | A | 86 | 117.812 | 133.802 | 55.768 | 1.00 | 36.83 | A |
| ATOM | 250 | CG | PRO | A | 86 | 116.694 | 134.063 | 54.770 | 1.00 | 35.53 | A |
| ATOM | 251 | C | PRO | A | 86 | 117.164 | 135.095 | 57.864 | 1.00 | 38.29 | A |
| ATOM | 252 | O | PRO | A | 86 | 118.166 | 135.402 | 58.455 | 1.00 | 39.78 | A |
| ATOM | 253 | N | ILE | A | 87 | 116.140 | 135.934 | 57.789 | 1.00 | 38.19 | A |
| ATOM | 254 | CA | ILE | A | 87 | 116.172 | 137.197 | 58.537 | 1.00 | 37.06 | A |
| ATOM | 255 | CB | ILE | A | 87 | 116.378 | 138.443 | 57.634 | 1.00 | 36.67 | A |
| ATOM | 256 | CG2 | ILE | A | 87 | 117.715 | 138.411 | 57.010 | 1.00 | 35.66 | A |
| ATOM | 257 | CG1 | ILE | A | 87 | 115.347 | 138.444 | 56.490 | 1.00 | 41.06 | A |
| ATOM | 258 | CD1 | ILE | A | 87 | 113.827 | 138.606 | 56.908 | 1.00 | 41.13 | A |
| ATOM | 259 | C | ILE | A | 87 | 114.779 | 137.268 | 59.111 | 1.00 | 35.68 | A |
| ATOM | 260 | O | ILE | A | 87 | 113.922 | 136.480 | 58.760 | 1.00 | 36.41 | A |
| ATOM | 261 | N | ALA | A | 88 | 114.537 | 138.197 | 60.011 | 1.00 | 35.17 | A |
| ATOM | 262 | CA | ALA | A | 88 | 113.178 | 138.351 | 60.534 | 1.00 | 34.45 | A |
| ATOM | 263 | CB | ALA | A | 88 | 113.231 | 138.810 | 61.982 | 1.00 | 32.89 | A |
| ATOM | 264 | C | ALA | A | 88 | 112.424 | 139.388 | 59.635 | 1.00 | 33.85 | A |
| ATOM | 265 | O | ALA | A | 88 | 112.859 | 140.542 | 59.432 | 1.00 | 33.60 | A |
| ATOM | 266 | N | PHE | A | 89 | 111.293 | 138.954 | 59.101 | 1.00 | 33.22 | A |
| ATOM | 267 | CA | PHE | A | 89 | 110.521 | 139.801 | 58.227 | 1.00 | 32.51 | A |
| ATOM | 268 | CB | PHE | A | 89 | 109.296 | 139.079 | 57.700 | 1.00 | 28.29 | A |
| ATOM | 269 | CG | PHE | A | 89 | 108.628 | 139.829 | 56.607 | 1.00 | 27.39 | A |
| ATOM | 270 | CD1 | PHE | A | 89 | 109.262 | 140.005 | 55.376 | 1.00 | 27.59 | A |
| ATOM | 271 | CD2 | PHE | A | 89 | 107.370 | 140.388 | 56.800 | 1.00 | 25.52 | A |
| ATOM | 272 | CE1 | PHE | A | 89 | 108.642 | 140.733 | 54.347 | 1.00 | 26.76 | A |
| ATOM | 273 | CE2 | PHE | A | 89 | 106.728 | 141.106 | 55.794 | 1.00 | 24.79 | A |
| ATOM | 274 | CZ | PHE | A | 89 | 107.367 | 141.283 | 54.555 | 1.00 | 26.52 | A |
| ATOM | 275 | C | PHE | A | 89 | 110.090 | 141.051 | 58.960 | 1.00 | 32.90 | A |
| ATOM | 276 | O | PHE | A | 89 | 109.602 | 140.953 | 60.070 | 1.00 | 35.42 | A |
| ATOM | 277 | N | LYS | A | 90 | 110.292 | 142.218 | 58.356 | 1.00 | 32.25 | A |
| ATOM | 278 | CA | LYS | A | 90 | 109.907 | 143.474 | 58.947 | 1.00 | 31.46 | A |
| ATOM | 279 | CB | LYS | A | 90 | 111.115 | 144.305 | 59.323 | 1.00 | 33.28 | A |
| ATOM | 280 | CG | LYS | A | 90 | 111.876 | 143.935 | 60.538 | 1.00 | 34.70 | A |
| ATOM | 281 | CD | LYS | A | 90 | 113.084 | 144.830 | 60.561 | 1.00 | 35.02 | A |
| ATOM | 282 | CE | LYS | A | 90 | 114.289 | 144.099 | 61.128 | 1.00 | 38.34 | A |
| ATOM | 283 | NZ | LYS | A | 90 | 114.130 | 143.916 | 62.562 | 1.00 | 37.83 | A |
| ATOM | 284 | C | LYS | A | 90 | 109.132 | 144.334 | 57.984 | 1.00 | 30.76 | A |
| ATOM | 285 | O | LYS | A | 90 | 109.446 | 144.363 | 56.797 | 1.00 | 30.55 | A |
| ATOM | 286 | N | VAL | A | 91 | 108.161 | 145.082 | 58.518 | 1.00 | 30.08 | A |
| ATOM | 287 | CA | VAL | A | 91 | 107.379 | 146.047 | 57.725 | 1.00 | 29.62 | A |
| ATOM | 288 | CB | VAL | A | 91 | 105.812 | 145.792 | 57.795 | 1.00 | 29.73 | A |
| ATOM | 289 | CG1 | VAL | A | 91 | 105.063 | 146.983 | 57.143 | 1.00 | 24.54 | A |
| ATOM | 290 | CG2 | VAL | A | 91 | 105.450 | 144.505 | 57.101 | 1.00 | 28.15 | A |
| ATOM | 291 | C | VAL | A | 91 | 107.685 | 147.438 | 58.333 | 1.00 | 29.02 | A |
| ATOM | 292 | O | VAL | A | 91 | 107.468 | 147.657 | 59.512 | 1.00 | 29.28 | A |
| ATOM | 293 | N | VAL | A | 92 | 108.220 | 148.347 | 57.528 | 1.00 | 28.54 | A |
| ATOM | 294 | CA | VAL | A | 92 | 108.564 | 149.677 | 57.990 | 1.00 | 27.03 | A |
| ATOM | 295 | CB | VAL | A | 92 | 110.000 | 150.037 | 57.574 | 1.00 | 26.69 | A |
| ATOM | 296 | CG1 | VAL | A | 92 | 110.351 | 151.436 | 57.998 | 1.00 | 23.89 | A |
| ATOM | 297 | CG2 | VAL | A | 92 | 110.959 | 149.009 | 58.150 | 1.00 | 25.25 | A |
| ATOM | 298 | C | VAL | A | 92 | 107.597 | 150.649 | 57.373 | 1.00 | 28.28 | A |
| ATOM | 299 | O | VAL | A | 92 | 107.392 | 150.624 | 56.128 | 1.00 | 26.66 | A |
| ATOM | 300 | N | ALA | A | 93 | 107.002 | 151.503 | 58.229 | 1.00 | 28.79 | A |
| ATOM | 301 | CA | ALA | A | 93 | 106.021 | 152.480 | 57.733 | 1.00 | 30.08 | A |
| ATOM | 302 | CB | ALA | A | 93 | 104.810 | 152.609 | 58.685 | 1.00 | 29.71 | A |
| ATOM | 303 | C | ALA | A | 93 | 106.733 | 153.811 | 57.564 | 1.00 | 30.91 | A |
| ATOM | 304 | O | ALA | A | 93 | 107.528 | 154.245 | 58.411 | 1.00 | 31.17 | A |
| ATOM | 305 | N | LEU | A | 94 | 106.476 | 154.442 | 56.432 | 1.00 | 32.23 | A |
| ATOM | 306 | CA | LEU | A | 94 | 107.055 | 155.726 | 56.156 | 1.00 | 31.89 | A |
| ATOM | 307 | CB | LEU | A | 94 | 106.978 | 155.941 | 54.665 | 1.00 | 31.11 | A |
| ATOM | 308 | CG | LEU | A | 94 | 107.927 | 154.960 | 53.945 | 1.00 | 31.48 | A |
| ATOM | 309 | CD1 | LEU | A | 94 | 107.964 | 155.281 | 52.464 | 1.00 | 30.59 | A |
| ATOM | 310 | CD2 | LEU | A | 94 | 109.338 | 155.051 | 54.503 | 1.00 | 29.60 | A |
| ATOM | 311 | C | LEU | A | 94 | 106.230 | 156.738 | 56.939 | 1.00 | 32.69 | A |
| ATOM | 312 | O | LEU | A | 94 | 106.712 | 157.266 | 57.971 | 1.00 | 32.92 | A |
| ATOM | 313 | N | GLY | A | 95 | 104.996 | 156.978 | 56.476 | 1.00 | 31.79 | A |
| ATOM | 314 | CA | GLY | A | 95 | 104.127 | 157.912 | 57.165 | 1.00 | 30.87 | A |
| ATOM | 315 | C | GLY | A | 95 | 103.909 | 157.486 | 58.603 | 1.00 | 31.22 | A |
| ATOM | 316 | O | GLY | A | 95 | 104.018 | 156.295 | 58.939 | 1.00 | 30.83 | A |
| ATOM | 317 | N | ASP | A | 96 | 103.634 | 158.459 | 59.466 | 1.00 | 31.42 | A |
| ATOM | 318 | CA | ASP | A | 96 | 103.388 | 158.173 | 60.881 | 1.00 | 32.72 | A |
| ATOM | 319 | CD | ASP | A | 96 | 102.881 | 159.439 | 61.564 | 1.00 | 34.83 | A |
| ATOM | 320 | CG | ASP | A | 96 | 103.961 | 160.493 | 61.730 | 1.00 | 40.38 | A |
| ATOM | 321 | OD1 | ASP | A | 96 | 104.922 | 160.484 | 60.897 | 1.00 | 44.25 | A |
| ATOM | 322 | OD2 | ASP | A | 96 | 103.864 | 161.343 | 62.676 | 1.00 | 39.79 | A |
| ATOM | 323 | C | ASP | A | 96 | 102.339 | 157.042 | 61.041 | 1.00 | 32.26 | A |
| ATOM | 324 | O | ASP | A | 96 | 101.305 | 157.020 | 60.341 | 1.00 | 31.14 | A |
| ATOM | 325 | N | VAL | A | 97 | 102.639 | 156.093 | 61.925 | 1.00 | 30.09 | A |
| ATOM | 326 | CA | VAL | A | 97 | 101.713 | 155.033 | 62.256 | 1.00 | 29.84 | A |
| ATOM | 327 | CB | VAL | A | 97 | 102.002 | 153.697 | 61.501 | 1.00 | 30.12 | A |
| ATOM | 328 | CG1 | VAL | A | 97 | 101.237 | 152.525 | 62.187 | 1.00 | 27.89 | A |
| ATOM | 329 | CG2 | VAL | A | 97 | 101.519 | 153.799 | 60.038 | 1.00 | 27.67 | A |
| ATOM | 330 | C | VAL | A | 97 | 101.808 | 154.770 | 63.762 | 1.00 | 29.76 | A |
| ATOM | 331 | O | VAL | A | 97 | 102.814 | 154.351 | 64.257 | 1.00 | 30.42 | A |
| ATOM | 332 | N | PRO | A | 98 | 100.729 | 154.965 | 64.497 | 1.00 | 30.14 | A |
| ATOM | 333 | CD | PRO | A | 98 | 99.336 | 154.974 | 64.004 | 1.00 | 29.77 | A |
| ATOM | 334 | CA | PRO | A | 98 | 100.786 | 154.726 | 65.949 | 1.00 | 30.64 | A |
| ATOM | 335 | CB | PRO | A | 98 | 99.327 | 154.875 | 66.414 | 1.00 | 29.34 | A |
| ATOM | 336 | CG | PRO | A | 98 | 98.576 | 155.489 | 65.216 | 1.00 | 28.04 | A |
| ATOM | 337 | C | PRO | A | 98 | 101.287 | 153.346 | 66.324 | 1.00 | 32.08 | A |
| ATOM | 338 | O | PRO | A | 98 | 101.003 | 152.345 | 65.651 | 1.00 | 32.48 | A |
| ATOM | 339 | N | ASP | A | 99 | 102.007 | 153.309 | 67.428 | 1.00 | 33.21 | A |
| ATOM | 340 | CA | ASP | A | 99 | 102.535 | 152.081 | 67.991 | 1.00 | 34.76 | A |
| ATOM | 341 | CB | ASP | A | 99 | 103.265 | 152.393 | 69.283 | 1.00 | 35.07 | A |
| ATOM | 342 | CG | ASP | A | 99 | 104.659 | 152.891 | 69.033 | 1.00 | 37.69 | A |
| ATOM | 343 | OD1 | ASP | A | 99 | 104.965 | 153.237 | 67.887 | 1.00 | 37.84 | A |
| ATOM | 344 | OD2 | ASP | A | 99 | 105.478 | 152.932 | 69.973 | 1.00 | 40.40 | A |
| ATOM | 345 | C | ASP | A | 99 | 101.360 | 151.204 | 68.284 | 1.00 | 34.02 | A |
| ATOM | 346 | O | ASP | A | 99 | 100.300 | 151.722 | 68.582 | 1.00 | 34.83 | A |
| ATOM | 347 | N | GLY | A | 100 | 101.519 | 149.897 | 68.155 | 1.00 | 33.00 | A |
| ATOM | 348 | CA | GLY | A | 100 | 100.414 | 149.009 | 68.464 | 1.00 | 31.62 | A |
| ATOM | 349 | C | GLY | A | 100 | 99.485 | 148.659 | 67.333 | 1.00 | 30.19 | A |
| ATOM | 350 | O | GLY | A | 100 | 98.619 | 147.821 | 67.466 | 1.00 | 28.94 | A |
| ATOM | 351 | N | THR | A | 101 | 99.644 | 149.332 | 66.217 | 1.00 | 30.51 | A |
| ATOM | 352 | CA | THR | A | 101 | 98.836 | 149.013 | 65.058 | 1.00 | 30.74 | A |
| ATOM | 353 | CB | THR | A | 101 | 99.106 | 149.956 | 63.970 | 1.00 | 30.13 | A |
| ATOM | 354 | OG1 | THR | A | 101 | 98.780 | 151.291 | 64.421 | 1.00 | 30.29 | A |
| ATOM | 355 | CG2 | THR | A | 101 | 98.348 | 149.556 | 62.749 | 1.00 | 28.54 | A |
| ATOM | 356 | C | THR | A | 101 | 99.226 | 147.606 | 64.583 | 1.00 | 31.92 | A |
| ATOM | 357 | O | THR | A | 101 | 100.425 | 147.238 | 64.505 | 1.00 | 33.81 | A |
| ATOM | 358 | N | LEU | A | 102 | 98.223 | 146.807 | 64.285 | 1.00 | 30.33 | A |
| ATOM | 359 | CA | LEU | A | 102 | 98.509 | 145.461 | 63.926 | 1.00 | 29.81 | A |
| ATOM | 360 | CB | LEU | A | 102 | 97.299 | 144.600 | 64.233 | 1.00 | 28.80 | A |
| ATOM | 361 | CG | LEU | A | 102 | 97.335 | 143.802 | 65.555 | 1.00 | 31.18 | A |
| ATOM | 362 | CD1 | LEU | A | 102 | 98.123 | 144.490 | 66.616 | 1.00 | 30.75 | A |
| ATOM | 363 | CD2 | LEU | A | 102 | 95.924 | 143.493 | 65.988 | 1.00 | 27.41 | A |
| ATOM | 364 | C | LEU | A | 102 | 98.872 | 145.413 | 62.492 | 1.00 | 30.09 | A |
| ATOM | 365 | O | LEU | A | 102 | 98.380 | 146.181 | 61.681 | 1.00 | 31.32 | A |
| ATOM | 366 | N | VAL | A | 103 | 99.734 | 144.483 | 62.169 | 1.00 | 29.07 | A |
| ATOM | 367 | CA | VAL | A | 103 | 100.145 | 144.314 | 60.811 | 1.00 | 26.99 | A |
| ATOM | 368 | CD | VAL | A | 103 | 101.548 | 144.850 | 60.640 | 1.00 | 26.69 | A |
| ATOM | 369 | CG1 | VAL | A | 103 | 102.016 | 144.665 | 59.181 | 1.00 | 26.35 | A |
| ATOM | 370 | CG2 | VAL | A | 103 | 101.595 | 146.278 | 61.069 | 1.00 | 24.23 | A |
| ATOM | 371 | C | VAL | A | 103 | 100.133 | 142.819 | 60.548 | 1.00 | 27.41 | A |
| ATOM | 372 | O | VAL | A | 103 | 100.610 | 142.020 | 61.396 | 1.00 | 26.34 | A |
| ATOM | 373 | N | THR | A | 104 | 99.592 | 142.434 | 59.386 | 1.00 | 27.10 | A |
| ATOM | 374 | CA | THR | A | 104 | 99.534 | 141.016 | 59.027 | 1.00 | 28.43 | A |
| ATOM | 375 | CB | THR | A | 104 | 98.119 | 140.459 | 59.081 | 1.00 | 28.47 | A |
| ATOM | 376 | OG1 | THR | A | 104 | 97.332 | 141.125 | 58.098 | 1.00 | 29.76 | A |
| ATOM | 377 | CG2 | THR | A | 104 | 97.487 | 140.691 | 60.411 | 1.00 | 26.65 | A |
| ATOM | 378 | C | THR | A | 104 | 100.029 | 140.752 | 57.631 | 1.00 | 29.56 | A |
| ATOM | 379 | O | THR | A | 104 | 100.066 | 141.642 | 56.776 | 1.00 | 30.20 | A |
| ATOM | 380 | N | VAL | A | 105 | 100.409 | 139.504 | 57.387 | 1.00 | 30.13 | A |
| ATOM | 381 | CA | VAL | A | 105 | 100.884 | 139.108 | 56.057 | 1.00 | 29.05 | A |
| ATOM | 382 | CB | VAL | A | 105 | 102.363 | 138.646 | 56.073 | 1.00 | 25.99 | A |
| ATOM | 383 | CG1 | VAL | A | 105 | 102.750 | 138.093 | 54.704 | 1.00 | 23.43 | A |
| ATOM | 384 | CG2 | VAL | A | 105 | 103.246 | 139.821 | 56.418 | 1.00 | 26.04 | A |
| ATOM | 385 | C | VAL | A | 105 | 100.014 | 137.950 | 55.570 | 1.00 | 30.06 | A |
| ATOM | 386 | O | VAL | A | 105 | 99.668 | 137.067 | 56.367 | 1.00 | 28.92 | A |
| ATOM | 387 | N | MET | A | 106 | 99.664 | 137.991 | 54.275 | 1.00 | 30.18 | A |
| ATOM | 388 | CA | MET | A | 106 | 98.872 | 136.946 | 53.598 | 1.00 | 31.32 | A |
| ATOM | 389 | CB | MET | A | 106 | 97.429 | 137.366 | 53.370 | 1.00 | 32.33 | A |
| ATOM | 390 | CG | MET | A | 106 | 96.650 | 137.707 | 54.606 | 1.00 | 33.99 | A |
| ATOM | 391 | SD | MET | A | 106 | 94.917 | 137.953 | 54.042 | 1.00 | 41.17 | A |
| ATOM | 392 | CE | MET | A | 106 | 94.724 | 139.723 | 54.230 | 1.00 | 36.87 | A |
| ATOM | 393 | C | MET | A | 106 | 99.537 | 136.766 | 52.247 | 1.00 | 31.72 | A |
| ATOM | 394 | O | MET | A | 106 | 100.194 | 137.684 | 51.705 | 1.00 | 31.63 | A |
| ATOM | 395 | N | ALA | A | 107 | 99.365 | 135.603 | 51.674 | 1.00 | 31.14 | A |
| ATOM | 396 | CA | ALA | A | 107 | 100.024 | 135.351 | 50.423 | 1.00 | 32.03 | A |
| ATOM | 397 | CB | ALA | A | 107 | 101.409 | 134.675 | 50.694 | 1.00 | 31.60 | A |
| ATOM | 398 | C | ALA | A | 107 | 99.137 | 134.488 | 49.521 | 1.00 | 33.51 | A |
| ATOM | 399 | O | ALA | A | 107 | 98.268 | 133.744 | 49.988 | 1.00 | 33.49 | A |
| ATOM | 400 | N | GLY | A | 108 | 99.354 | 134.627 | 48.218 | 1.00 | 33.85 | A |
| ATOM | 401 | CA | GLY | A | 108 | 98.607 | 133.876 | 47.246 | 1.00 | 33.73 | A |
| ATOM | 402 | C | GLY | A | 108 | 99.190 | 134.118 | 45.883 | 1.00 | 34.90 | A |
| ATOM | 403 | O | GLY | A | 108 | 100.103 | 134.912 | 45.712 | 1.00 | 35.29 | A |
| ATOM | 404 | N | ASN | A | 109 | 98.707 | 133.385 | 44.899 | 1.00 | 36.51 | A |
| ATOM | 405 | CA | ASN | A | 109 | 99.169 | 133.601 | 43.542 | 1.00 | 38.15 | A |
| ATOM | 406 | CB | ASN | A | 109 | 100.619 | 133.171 | 43.338 | 1.00 | 37.15 | A |
| ATOM | 407 | CG | ASN | A | 109 | 100.773 | 131.699 | 43.291 | 1.00 | 38.22 | A |
| ATOM | 408 | OD1 | ASN | A | 109 | 99.812 | 130.950 | 43.407 | 1.00 | 38.68 | A |
| ATOM | 409 | ND2 | ASN | A | 109 | 102.004 | 131.258 | 43.146 | 1.00 | 39.89 | A |
| ATOM | 410 | C | ASN | A | 109 | 98.221 | 132.824 | 42.653 | 1.00 | 39.82 | A |
| ATOM | 411 | O | ASN | A | 109 | 97.256 | 132.241 | 43.165 | 1.00 | 39.93 | A |
| ATOM | 412 | N | ASP | A | 110 | 98.484 | 132.791 | 41.348 | 1.00 | 41.82 | A |
| ATOM | 413 | CA | ASP | A | 110 | 97.526 | 132.126 | 40.454 | 1.00 | 45.07 | A |
| ATOM | 414 | CB | ASP | A | 110 | 97.713 | 132.586 | 38.981 | 1.00 | 45.78 | A |
| ATOM | 415 | CG | ASP | A | 110 | 98.916 | 131.959 | 38.310 | 1.00 | 47.99 | A |
| ATOM | 416 | OD1 | ASP | A | 110 | 99.087 | 132.201 | 37.106 | 1.00 | 48.13 | A |
| ATOM | 417 | OD2 | ASP | A | 110 | 99.690 | 131.233 | 38.973 | 1.00 | 50.27 | A |
| ATOM | 418 | C | ASP | A | 110 | 97.476 | 130.600 | 40.510 | 1.00 | 45.42 | A |
| ATOM | 419 | O | ASP | A | 110 | 96.884 | 129.957 | 39.643 | 1.00 | 45.69 | A |
| ATOM | 420 | N | GLU | A | 111 | 98.073 | 130.031 | 41.543 | 1.00 | 45.46 | A |
| ATOM | 421 | CA | GLU | A | 111 | 98.084 | 128.598 | 41.691 | 1.00 | 45.64 | A |
| ATOM | 422 | CB | GLU | A | 111 | 99.500 | 128.094 | 41.423 | 1.00 | 47.12 | A |
| ATOM | 423 | CG | GLU | A | 111 | 99.612 | 126.607 | 41.515 | 1.00 | 50.03 | A |
| ATOM | 424 | CD | GLU | A | 111 | 100.569 | 126.046 | 40.516 | 1.00 | 52.26 | A |
| ATOM | 425 | OE1 | GLU | A | 111 | 101.074 | 124.961 | 40.807 | 1.00 | 54.90 | A |
| ATOM | 426 | OE2 | GLU | A | 111 | 100.817 | 126.661 | 39.446 | 1.00 | 53.45 | A |
| ATOM | 427 | C | GLU | A | 111 | 97.592 | 128.173 | 43.077 | 1.00 | 45.33 | A |
| ATOM | 428 | O | GLU | A | 111 | 96.895 | 127.188 | 43.217 | 1.00 | 46.23 | A |
| ATOM | 429 | N | ASN | A | 112 | 97.966 | 128.921 | 44.103 | 1.00 | 43.82 | A |
| ATOM | 430 | CA | ASN | A | 112 | 97.556 | 128.625 | 45.456 | 1.00 | 43.02 | A |
| ATOM | 431 | CB | ASN | A | 112 | 98.795 | 128.409 | 46.296 | 1.00 | 41.84 | A |
| ATOM | 432 | CG | ASN | A | 112 | 98.481 | 128.011 | 47.681 | 1.00 | 41.49 | A |
| ATOM | 433 | OD1 | ASN | A | 112 | 97.320 | 128.173 | 48.155 | 1.00 | 40.84 | A |
| ATOM | 434 | ND2 | ASN | A | 112 | 99.507 | 127.503 | 48.386 | 1.00 | 39.01 | A |
| ATOM | 435 | C | ASN | A | 112 | 96.902 | 129.963 | 45.717 | 1.00 | 43.18 | A |
| ATOM | 436 | O | ASN | A | 112 | 97.572 | 130.992 | 45.766 | 1.00 | 43.99 | A |
| ATOM | 437 | N | TYR | A | 113 | 95.590 | 129.981 | 45.831 | 1.00 | 42.34 | A |
| ATOM | 438 | CA | TYR | A | 113 | 94.937 | 131.266 | 45.954 | 1.00 | 41.65 | A |
| ATOM | 439 | CB | TYR | A | 113 | 93.463 | 131.140 | 45.541 | 1.00 | 42.19 | A |
| ATOM | 440 | CG | TYR | A | 113 | 92.570 | 130.526 | 46.587 | 1.00 | 43.41 | A |
| ATOM | 441 | CD1 | TYR | A | 113 | 91.955 | 131.332 | 47.530 | 1.00 | 43.39 | A |
| ATOM | 442 | CE1 | TYR | A | 113 | 91.189 | 130.816 | 48.521 | 1.00 | 44.11 | A |
| ATOM | 443 | CD2 | TYR | A | 113 | 92.378 | 129.147 | 46.665 | 1.00 | 44.11 | A |
| ATOM | 444 | CE2 | TYR | A | 113 | 91.589 | 128.598 | 47.681 | 1.00 | 45.55 | A |
| ATOM | 445 | CZ | TYR | A | 113 | 91.009 | 129.456 | 48.614 | 1.00 | 46.23 | A |
| ATOM | 446 | OH | TYR | A | 113 | 90.317 | 128.985 | 49.720 | 1.00 | 47.53 | A |
| ATOM | 447 | C | TYR | A | 113 | 95.111 | 131.864 | 47.337 | 1.00 | 41.30 | A |
| ATOM | 448 | O | TYR | A | 113 | 95.000 | 133.069 | 47.542 | 1.00 | 41.14 | A |
| ATOM | 449 | N | SER | A | 114 | 95.426 | 131.020 | 48.296 | 1.00 | 41.04 | A |
| ATOM | 450 | CA | SER | A | 114 | 95.647 | 131.515 | 49.649 | 1.00 | 40.02 | A |
| ATOM | 451 | CB | SER | A | 114 | 94.349 | 131.605 | 50.370 | 1.00 | 39.19 | A |
| ATOM | 452 | OG | SER | A | 114 | 94.552 | 132.325 | 51.546 | 1.00 | 42.35 | A |
| ATOM | 453 | C | SER | A | 114 | 96.609 | 130.593 | 50.419 | 1.00 | 38.98 | A |
| ATOM | 454 | O | SER | A | 114 | 96.216 | 129.561 | 50.964 | 1.00 | 39.32 | A |
| ATOM | 455 | N | ALA | A | 115 | 97.872 | 130.977 | 50.470 | 1.00 | 36.99 | A |
| ATOM | 456 | CA | ALA | A | 115 | 98.856 | 130.147 | 51.114 | 1.00 | 36.02 | A |
| ATOM | 457 | CB | ALA | A | 115 | 100.248 | 130.580 | 50.647 | 1.00 | 37.65 | A |
| ATOM | 458 | C | ALA | A | 115 | 98.784 | 130.163 | 52.628 | 1.00 | 35.88 | A |
| ATOM | 459 | O | ALA | A | 115 | 98.559 | 131.196 | 53.251 | 1.00 | 35.16 | A |
| ATOM | 460 | N | GLU | A | 116 | 98.994 | 129.004 | 53.216 | 1.00 | 35.23 | A |
| ATOM | 461 | CA | GLU | A | 116 | 99.004 | 128.870 | 54.659 | 1.00 | 36.72 | A |
| ATOM | 462 | CB | GLU | A | 116 | 98.886 | 127.397 | 55.044 | 1.00 | 39.60 | A |
| ATOM | 463 | CG | GLU | A | 116 | 99.542 | 127.057 | 56.342 | 1.00 | 44.01 | A |
| ATOM | 464 | CD | GLU | A | 116 | 99.129 | 125.697 | 56.838 | 1.00 | 47.77 | A |
| ATOM | 465 | OE1 | GLU | A | 116 | 99.663 | 124.640 | 56.385 | 1.00 | 49.48 | A |
| ATOM | 466 | OE2 | GLU | A | 116 | 98.228 | 125.692 | 57.699 | 1.00 | 51.55 | A |
| ATOM | 467 | C | GLU | A | 116 | 100.299 | 129.439 | 55.235 | 1.00 | 35.82 | A |
| ATOM | 468 | O | GLU | A | 116 | 101.390 | 129.094 | 54.785 | 1.00 | 34.75 | A |
| ATOM | 469 | N | LEU | A | 117 | 100.162 | 130.279 | 56.250 | 1.00 | 34.58 | A |
| ATOM | 470 | CA | LEU | A | 117 | 101.300 | 130.930 | 56.863 | 1.00 | 34.27 | A |
| ATOM | 471 | CB | LEU | A | 117 | 101.174 | 132.427 | 56.691 | 1.00 | 32.15 | A |
| ATOM | 472 | CG | LEU | A | 117 | 101.081 | 132.945 | 55.282 | 1.00 | 32.64 | A |
| ATOM | 473 | CD1 | LEU | A | 117 | 101.082 | 134.471 | 55.409 | 1.00 | 31.94 | A |
| ATOM | 474 | CD2 | LEU | A | 117 | 102.269 | 132.444 | 54.417 | 1.00 | 30.26 | A |
| ATOM | 475 | C | LEU | A | 117 | 101.344 | 130.660 | 58.345 | 1.00 | 34.78 | A |
| ATOM | 476 | O | LEU | A | 117 | 100.414 | 130.110 | 58.888 | 1.00 | 35.76 | A |
| ATOM | 477 | N | ARG | A | 118 | 102.414 | 131.079 | 59.007 | 1.00 | 35.03 | A |
| ATOM | 478 | CA | ARG | A | 118 | 102.531 | 130.910 | 60.450 | 1.00 | 34.96 | A |
| ATOM | 479 | CB | ARG | A | 118 | 103.363 | 129.681 | 60.784 | 1.00 | 36.58 | A |
| ATOM | 480 | CG | ARG | A | 118 | 102.633 | 128.377 | 60.589 | 1.00 | 40.84 | A |
| ATOM | 481 | CD | ARG | A | 118 | 103.572 | 127.195 | 60.880 | 1.00 | 44.85 | A |
| ATOM | 482 | NE | ARG | A | 118 | 104.342 | 127.469 | 62.093 | 1.00 | 49.35 | A |
| ATOM | 483 | CZ | ARG | A | 118 | 105.237 | 126.643 | 62.641 | 1.00 | 51.15 | A |
| ATOM | 484 | NH1 | ARG | A | 118 | 105.481 | 125.469 | 62.069 | 1.00 | 51.84 | A |
| ATOM | 485 | NH2 | ARG | A | 118 | 105.873 | 126.987 | 63.776 | 1.00 | 51.86 | A |
| ATOM | 486 | C | ARG | A | 118 | 103.160 | 132.184 | 61.048 | 1.00 | 34.33 | A |
| ATOM | 487 | O | ARG | A | 118 | 104.130 | 132.770 | 60.504 | 1.00 | 34.31 | A |
| ATOM | 488 | N | ASN | A | 119 | 102.589 | 132.623 | 62.165 | 1.00 | 31.96 | A |
| ATOM | 489 | CA | ASN | A | 119 | 103.056 | 133.837 | 62.831 | 1.00 | 30.50 | A |
| ATOM | 490 | CB | ASN | A | 119 | 104.493 | 133.664 | 63.241 | 1.00 | 31.53 | A |
| ATOM | 491 | CG | ASN | A | 119 | 104.730 | 132.301 | 63.875 | 1.00 | 33.12 | A |
| ATOM | 492 | OD1 | ASN | A | 119 | 103.903 | 131.841 | 64.695 | 1.00 | 32.56 | A |
| ATOM | 493 | ND2 | ASN | A | 119 | 105.849 | 131.648 | 63.519 | 1.00 | 31.63 | A |
| ATOM | 494 | C | ASN | A | 119 | 102.958 | 135.013 | 61.917 | 1.00 | 29.61 | A |
| ATOM | 495 | O | ASN | A | 119 | 103.866 | 135.806 | 61.879 | 1.00 | 30.73 | A |
| ATOM | 496 | N | ALA | A | 120 | 101.855 | 135.135 | 61.177 | 1.00 | 28.86 | A |
| ATOM | 497 | CA | ALA | A | 120 | 101.675 | 136.228 | 60.249 | 1.00 | 28.03 | A |
| ATOM | 498 | CB | ALA | A | 120 | 100.865 | 135.811 | 59.080 | 1.00 | 26.43 | A |
| ATOM | 499 | C | ALA | A | 120 | 101.082 | 137.495 | 60.811 | 1.00 | 28.96 | A |
| ATOM | 500 | O | ALA | A | 120 | 100.566 | 138.311 | 60.028 | 1.00 | 29.95 | A |
| ATOM | 501 | N | THR | A | 121 | 101.103 | 137.691 | 62.139 | 1.00 | 29.29 | A |
| ATOM | 502 | CA | THR | A | 121 | 100.594 | 138.973 | 62.726 | 1.00 | 28.76 | A |
| ATOM | 503 | CB | THR | A | 121 | 99.422 | 138.811 | 63.716 | 1.00 | 29.22 | A |
| ATOM | 504 | OG1 | THR | A | 121 | 98.356 | 138.055 | 63.118 | 1.00 | 28.08 | A |
| ATOM | 505 | CG2 | THR | A | 121 | 98.884 | 140.217 | 64.127 | 1.00 | 28.11 | A |
| ATOM | 506 | C | THR | A | 121 | 101.694 | 139.650 | 63.523 | 1.00 | 27.61 | A |
| ATOM | 507 | O | THR | A | 121 | 102.479 | 138.981 | 64.154 | 1.00 | 28.82 | A |
| ATOM | 508 | N | ALA | A | 122 | 101.776 | 140.961 | 63.471 | 1.00 | 26.60 | A |
| ATOM | 509 | CA | ALA | A | 122 | 102.800 | 141.685 | 64.230 | 1.00 | 28.21 | A |
| ATOM | 510 | CB | ALA | A | 122 | 104.132 | 141.803 | 63.408 | 1.00 | 26.58 | A |
| ATOM | 511 | C | ALA | A | 122 | 102.287 | 143.063 | 64.630 | 1.00 | 28.51 | A |
| ATOM | 512 | O | ALA | A | 122 | 101.196 | 143.483 | 64.212 | 1.00 | 28.45 | A |
| ATOM | 513 | N | ALA | A | 123 | 103.059 | 143.785 | 65.437 | 1.00 | 29.51 | A |
| ATOM | 514 | CA | ALA | A | 123 | 102.561 | 145.096 | 65.871 | 1.00 | 30.46 | A |
| ATOM | 515 | CB | ALA | A | 123 | 102.145 | 145.034 | 67.344 | 1.00 | 28.39 | A |
| ATOM | 516 | C | ALA | A | 123 | 103.599 | 146.156 | 65.649 | 1.00 | 31.56 | A |
| ATOM | 517 | O | ALA | A | 123 | 104.793 | 145.910 | 65.809 | 1.00 | 32.00 | A |
| ATOM | 518 | N | MET | A | 124 | 103.138 | 147.331 | 65.245 | 1.00 | 32.39 | A |
| ATOM | 519 | CA | MET | A | 124 | 104.030 | 148.451 | 64.975 | 1.00 | 32.26 | A |
| ATOM | 520 | CB | MET | A | 124 | 103.253 | 149.590 | 64.296 | 1.00 | 33.10 | A |
| ATOM | 521 | CG | MET | A | 124 | 103.143 | 149.532 | 62.804 | 1.00 | 32.96 | A |
| ATOM | 522 | SD | MET | A | 124 | 104.768 | 149.797 | 61.985 | 1.00 | 33.45 | A |
| ATOM | 523 | CE | MET | A | 124 | 104.210 | 149.760 | 60.273 | 1.00 | 31.54 | A |
| ATOM | 524 | C | MET | A | 124 | 104.617 | 149.002 | 66.256 | 1.00 | 33.14 | A |
| ATOM | 525 | O | MET | A | 124 | 103.914 | 149.184 | 67.257 | 1.00 | 32.47 | A |
| ATOM | 526 | N | LYS | A | 125 | 105.924 | 149.236 | 66.225 | 1.00 | 34.67 | A |
| ATOM | 527 | CA | LYS | A | 125 | 106.651 | 149.858 | 67.330 | 1.00 | 35.15 | A |
| ATOM | 528 | CB | LYS | A | 125 | 107.343 | 148.850 | 68.217 | 1.00 | 34.40 | A |
| ATOM | 529 | CG | LYS | A | 125 | 108.014 | 149.584 | 69.299 | 1.00 | 37.92 | A |
| ATOM | 530 | CD | LYS | A | 125 | 108.258 | 148.736 | 70.535 | 1.00 | 42.82 | A |
| ATOM | 531 | CE | LYS | A | 125 | 108.429 | 149.667 | 71.777 | 1.00 | 45.40 | A |
| ATOM | 532 | NZ | LYS | A | 125 | 108.412 | 148.958 | 73.116 | 1.00 | 49.52 | A |
| ATOM | 533 | C | LYS | A | 125 | 107.692 | 150.752 | 66.660 | 1.00 | 34.91 | A |
| ATOM | 534 | O | LYS | A | 125 | 108.586 | 150.250 | 65.965 | 1.00 | 34.22 | A |
| ATOM | 535 | N | ASN | A | 126 | 107.558 | 152.061 | 66.855 | 1.00 | 33.18 | A |
| ATOM | 536 | CA | ASN | A | 126 | 108.469 | 153.001 | 66.236 | 1.00 | 33.10 | A |
| ATOM | 537 | CB | ASN | A | 126 | 109.882 | 152.828 | 66.789 | 1.00 | 33.71 | A |
| ATOM | 538 | CG | ASN | A | 126 | 109.962 | 153.136 | 68.264 | 1.00 | 34.27 | A |
| ATOM | 539 | OD1 | ASN | A | 126 | 109.419 | 154.110 | 68.718 | 1.00 | 29.72 | A |
| ATOM | 540 | ND2 | ASN | A | 126 | 110.671 | 152.287 | 69.015 | 1.00 | 36.23 | A |
| ATOM | 541 | C | ASN | A | 126 | 108.528 | 152.887 | 64.695 | 1.00 | 31.94 | A |
| ATOM | 542 | O | ASN | A | 126 | 109.638 | 152.897 | 64.074 | 1.00 | 30.43 | A |
| ATOM | 543 | N | GLN | A | 127 | 107.350 | 152.770 | 64.091 | 1.00 | 30.00 | A |
| ATOM | 544 | CA | GLN | A | 127 | 107.262 | 152.694 | 62.637 | 1.00 | 30.04 | A |
| ATOM | 545 | CB | GLN | A | 127 | 108.076 | 153.852 | 62.010 | 1.00 | 29.32 | A |
| ATOM | 546 | CG | GLN | A | 127 | 107.580 | 155.240 | 62.360 | 1.00 | 28.80 | A |
| ATOM | 547 | CD | GLN | A | 127 | 106.251 | 155.540 | 61.709 | 1.00 | 32.99 | A |
| ATOM | 548 | OE1 | GLN | A | 127 | 105.258 | 155.829 | 62.393 | 1.00 | 34.64 | A |
| ATOM | 549 | NE2 | GLN | A | 127 | 106.205 | 155.471 | 60.367 | 1.00 | 34.30 | A |
| ATOM | 550 | C | GLN | A | 127 | 107.718 | 151.349 | 62.045 | 1.00 | 30.04 | A |
| ATOM | 551 | O | GLN | A | 127 | 107.759 | 151.182 | 60.813 | 1.00 | 31.54 | A |
| ATOM | 552 | N | VAL | A | 128 | 108.051 | 150.397 | 62.903 | 1.00 | 28.39 | A |
| ATOM | 553 | CA | VAL | A | 128 | 108.504 | 149.111 | 62.429 | 1.00 | 28.19 | A |
| ATOM | 554 | CB | VAL | A | 128 | 109.993 | 148.902 | 62.758 | 1.00 | 27.28 | A |
| ATOM | 555 | CG1 | VAL | A | 128 | 110.377 | 147.445 | 62.566 | 1.00 | 21.75 | A |
| ATOM | 556 | CG2 | VAL | A | 128 | 110.813 | 149.819 | 61.912 | 1.00 | 27.05 | A |
| ATOM | 557 | C | VAL | A | 128 | 107.719 | 147.942 | 63.035 | 1.00 | 29.22 | A |
| ATOM | 558 | O | VAL | A | 128 | 107.594 | 147.813 | 64.254 | 1.00 | 30.31 | A |
| ATOM | 559 | N | ALA | A | 129 | 107.186 | 147.082 | 62.192 | 1.00 | 29.27 | A |
| ATOM | 560 | CA | ALA | A | 129 | 106.461 | 145.960 | 62.723 | 1.00 | 30.29 | A |
| ATOM | 561 | CB | ALA | A | 129 | 105.155 | 145.793 | 61.988 | 1.00 | 31.50 | A |
| ATOM | 562 | C | ALA | A | 129 | 107.366 | 144.780 | 62.457 | 1.00 | 30.33 | A |
| ATOM | 563 | O | ALA | A | 129 | 107.511 | 144.401 | 61.322 | 1.00 | 31.81 | A |
| ATOM | 564 | N | ARG | A | 130 | 107.973 | 144.206 | 63.493 | 1.00 | 30.33 | A |
| ATOM | 565 | CA | ARG | A | 130 | 108.861 | 143.072 | 63.320 | 1.00 | 31.35 | A |
| ATOM | 566 | CB | ARG | A | 130 | 110.034 | 143.191 | 64.301 | 1.00 | 31.56 | A |
| ATOM | 567 | CG | ARG | A | 130 | 110.942 | 141.984 | 64.332 | 1.00 | 34.78 | A |
| ATOM | 568 | CD | ARG | A | 130 | 112.087 | 142.172 | 65.284 | 1.00 | 37.60 | A |
| ATOM | 569 | NE | ARG | A | 130 | 113.072 | 141.092 | 65.181 | 1.00 | 42.86 | A |
| ATOM | 570 | CZ | ARG | A | 130 | 112.887 | 139.839 | 65.629 | 1.00 | 46.81 | A |
| ATOM | 571 | NH1 | ARG | A | 130 | 111.734 | 139.477 | 66.240 | 1.00 | 49.06 | A |
| ATOM | 572 | NH2 | ARG | A | 130 | 113.842 | 138.923 | 65.456 | 1.00 | 46.30 | A |
| ATOM | 573 | C | ARG | A | 130 | 108.168 | 141.722 | 63.519 | 1.00 | 30.39 | A |
| ATOM | 574 | O | ARG | A | 130 | 107.630 | 141.455 | 64.562 | 1.00 | 28.98 | A |
| ATOM | 575 | N | PHE | A | 131 | 108.189 | 140.863 | 62.519 | 1.00 | 30.26 | A |
| ATOM | 576 | CA | PHE | A | 131 | 107.583 | 139.573 | 62.702 | 1.00 | 30.41 | A |
| ATOM | 577 | CB | PHE | A | 131 | 107.125 | 139.001 | 61.400 | 1.00 | 26.53 | A |
| ATOM | 578 | CG | PHE | A | 131 | 105.973 | 139.677 | 60.856 | 1.00 | 25.23 | A |
| ATOM | 579 | CD1 | PHE | A | 131 | 106.112 | 140.935 | 60.275 | 1.00 | 23.98 | A |
| ATOM | 580 | CD2 | PHE | A | 131 | 104.711 | 139.087 | 60.942 | 1.00 | 25.26 | A |
| ATOM | 581 | CE1 | PHE | A | 131 | 105.016 | 141.627 | 59.773 | 1.00 | 23.37 | A |
| ATOM | 582 | CE2 | PHE | A | 131 | 103.577 | 139.763 | 60.438 | 1.00 | 26.67 | A |
| ATOM | 583 | CZ | PHE | A | 131 | 103.752 | 141.071 | 59.840 | 1.00 | 26.54 | A |
| ATOM | 584 | C | PHE | A | 131 | 108.565 | 138.620 | 63.302 | 1.00 | 33.30 | A |
| ATOM | 585 | O | PHE | A | 131 | 109.689 | 138.556 | 62.899 | 1.00 | 35.71 | A |
| ATOM | 586 | N | ASN | A | 132 | 108.124 | 137.882 | 64.292 | 1.00 | 36.51 | A |
| ATOM | 587 | CA | ASN | A | 132 | 108.931 | 136.871 | 64.948 | 1.00 | 38.43 | A |
| ATOM | 588 | CB | ASN | A | 132 | 108.477 | 136.736 | 66.395 | 1.00 | 39.85 | A |
| ATOM | 589 | CG | ASN | A | 132 | 109.415 | 135.839 | 67.198 | 1.00 | 45.67 | A |
| ATOM | 590 | OD1 | ASN | A | 132 | 110.651 | 136.130 | 67.317 | 1.00 | 47.58 | A |
| ATOM | 591 | ND2 | ASN | A | 132 | 108.863 | 134.723 | 67.741 | 1.00 | 45.40 | A |
| ATOM | 592 | C | ASN | A | 132 | 108.628 | 135.547 | 64.219 | 1.00 | 39.89 | A |
| ATOM | 593 | O | ASN | A | 132 | 107.446 | 135.101 | 64.161 | 1.00 | 42.90 | A |
| ATOM | 594 | N | ASP | A | 133 | 109.630 | 134.904 | 63.642 | 1.00 | 37.08 | A |
| ATOM | 595 | CA | ASP | A | 133 | 109.344 | 133.634 | 62.949 | 1.00 | 34.95 | A |
| ATOM | 596 | CB | ASP | A | 133 | 109.092 | 132.539 | 63.961 | 1.00 | 34.53 | A |
| ATOM | 597 | CG | ASP | A | 133 | 109.539 | 131.196 | 63.457 | 1.00 | 37.62 | A |
| ATOM | 598 | OD1 | ASP | A | 133 | 110.447 | 131.120 | 62.577 | 1.00 | 38.36 | A |
| ATOM | 599 | OD2 | ASP | A | 133 | 109.010 | 130.187 | 63.953 | 1.00 | 39.52 | A |
| ATOM | 600 | C | ASP | A | 133 | 108.253 | 133.503 | 61.871 | 1.00 | 33.32 | A |
| ATOM | 601 | O | ASP | A | 133 | 107.603 | 132.477 | 61.786 | 1.00 | 33.73 | A |
| ATOM | 602 | N | LEU | A | 134 | 108.029 | 134.518 | 61.055 | 1.00 | 31.79 | A |
| ATOM | 603 | CA | LEU | A | 134 | 107.068 | 134.384 | 59.956 | 1.00 | 29.83 | A |
| ATOM | 604 | CB | LEU | A | 134 | 107.061 | 135.656 | 59.091 | 1.00 | 29.25 | A |
| ATOM | 605 | CG | LEU | A | 134 | 106.403 | 135.516 | 57.680 | 1.00 | 31.00 | A |
| ATOM | 606 | CD1 | LEU | A | 134 | 104.899 | 135.255 | 57.803 | 1.00 | 30.58 | A |
| ATOM | 607 | CD2 | LEU | A | 134 | 106.592 | 136.774 | 56.856 | 1.00 | 27.36 | A |
| ATOM | 608 | C | LEU | A | 134 | 107.498 | 133.196 | 59.069 | 1.00 | 29.03 | A |
| ATOM | 609 | O | LEU | A | 134 | 108.675 | 133.050 | 58.725 | 1.00 | 26.90 | A |
| ATOM | 610 | N | ARG | A | 135 | 106.538 | 132.347 | 58.690 | 1.00 | 30.67 | A |
| ATOM | 611 | CA | ARG | A | 135 | 106.817 | 131.171 | 57.840 | 1.00 | 31.52 | A |
| ATOM | 612 | CB | ARG | A | 135 | 106.930 | 129.850 | 58.635 | 1.00 | 32.01 | A |
| ATOM | 613 | CG | ARG | A | 135 | 107.950 | 129.855 | 59.725 | 1.00 | 34.02 | A |
| ATOM | 614 | CD | ARG | A | 135 | 108.289 | 128.483 | 60.161 | 1.00 | 36.04 | A |
| ATOM | 615 | NE | ARG | A | 135 | 108.892 | 128.536 | 61.486 | 1.00 | 38.78 | A |
| ATOM | 616 | CZ | ARG | A | 135 | 109.372 | 127.473 | 62.127 | 1.00 | 40.74 | A |
| ATOM | 617 | NH1 | ARG | A | 135 | 109.326 | 126.252 | 61.553 | 1.00 | 40.25 | A |
| ATOM | 618 | NH2 | ARG | A | 135 | 109.882 | 127.622 | 63.356 | 1.00 | 40.09 | A |
| ATOM | 619 | C | ARG | A | 135 | 105.775 | 130.932 | 56.792 | 1.00 | 31.12 | A |
| ATOM | 620 | O | ARG | A | 135 | 104.567 | 131.166 | 56.997 | 1.00 | 30.77 | A |
| ATOM | 621 | N | PHE | A | 136 | 106.267 | 130.428 | 55.671 | 1.00 | 29.93 | A |
| ATOM | 622 | CA | PHE | A | 136 | 105.417 | 130.070 | 54.575 | 1.00 | 29.72 | A |
| ATOM | 623 | CB | PHE | A | 136 | 106.039 | 130.539 | 53.278 | 1.00 | 27.22 | A |
| ATOM | 624 | CG | PHE | A | 136 | 106.153 | 132.008 | 53.194 | 1.00 | 27.48 | A |
| ATOM | 625 | CD1 | PHE | A | 136 | 107.229 | 132.672 | 53.779 | 1.00 | 28.36 | A |
| ATOM | 626 | CD2 | PHE | A | 136 | 105.192 | 132.754 | 52.554 | 1.00 | 26.31 | A |
| ATOM | 627 | CE1 | PHE | A | 136 | 107.338 | 134.080 | 53.713 | 1.00 | 27.37 | A |
| ATOM | 628 | CE2 | PHE | A | 136 | 105.294 | 134.143 | 52.495 | 1.00 | 27.03 | A |
| ATOM | 629 | CZ | PHE | A | 136 | 106.346 | 134.806 | 53.060 | 1.00 | 26.16 | A |
| ATOM | 630 | C | PHE | A | 136 | 105.239 | 128.562 | 54.576 | 1.00 | 29.83 | A |
| ATOM | 631 | O | PHE | A | 136 | 106.192 | 127.822 | 54.451 | 1.00 | 30.90 | A |
| ATOM | 632 | N | VAL | A | 137 | 104.019 | 128.103 | 54.742 | 1.00 | 30.96 | A |
| ATOM | 633 | CA | VAL | A | 137 | 103.749 | 126.665 | 54.739 | 1.00 | 32.78 | A |
| ATOM | 634 | CB | VAL | A | 137 | 102.729 | 126.301 | 55.846 | 1.00 | 32.50 | A |
| ATOM | 635 | CG1 | VAL | A | 137 | 102.435 | 124.814 | 55.837 | 1.00 | 32.02 | A |
| ATOM | 636 | CG2 | VAL | A | 137 | 103.285 | 126.736 | 57.206 | 1.00 | 30.61 | A |
| ATOM | 637 | C | VAL | A | 137 | 103.216 | 126.213 | 53.361 | 1.00 | 34.14 | A |
| ATOM | 638 | O | VAL | A | 137 | 103.619 | 125.176 | 52.833 | 1.00 | 34.45 | A |
| ATOM | 639 | N | GLY | A | 138 | 102.298 | 126.981 | 52.790 | 1.00 | 33.98 | A |
| ATOM | 640 | CA | GLY | A | 138 | 101.767 | 126.626 | 51.489 | 1.00 | 35.37 | A |
| ATOM | 641 | C | GLY | A | 138 | 102.787 | 126.726 | 50.362 | 1.00 | 37.81 | A |
| ATOM | 642 | O | GLY | A | 138 | 103.646 | 127.585 | 50.385 | 1.00 | 38.72 | A |
| ATOM | 643 | N | ARG | A | 139 | 102.701 | 125.847 | 49.367 | 1.00 | 39.72 | A |
| ATOM | 644 | CA | ARG | A | 139 | 103.618 | 125.860 | 48.246 | 1.00 | 41.47 | A |
| ATOM | 645 | CB | ARG | A | 139 | 103.669 | 124.485 | 47.591 | 1.00 | 45.70 | A |
| ATOM | 646 | CG | ARG | A | 139 | 103.826 | 123.320 | 48.529 | 1.00 | 50.09 | A |
| ATOM | 647 | CD | ARG | A | 139 | 103.187 | 122.028 | 47.914 | 1.00 | 53.96 | A |
| ATOM | 648 | NE | ARG | A | 139 | 102.908 | 121.018 | 48.958 | 1.00 | 57.92 | A |
| ATOM | 649 | CZ | ARG | A | 139 | 102.669 | 121.281 | 50.261 | 1.00 | 58.76 | A |
| ATOM | 650 | NH1 | ARG | A | 139 | 102.662 | 122.531 | 50.770 | 1.00 | 58.09 | A |
| ATOM | 651 | NH2 | ARG | A | 139 | 102.428 | 120.262 | 51.076 | 1.00 | 59.51 | A |
| ATOM | 652 | C | ARG | A | 139 | 103.130 | 126.870 | 47.204 | 1.00 | 41.67 | A |
| ATOM | 653 | O | ARG | A | 139 | 101.918 | 127.106 | 47.063 | 1.00 | 41.68 | A |
| ATOM | 654 | N | SER | A | 140 | 104.067 | 127.442 | 46.448 | 1.00 | 40.23 | A |
| ATOM | 655 | CA | SER | A | 140 | 103.702 | 128.439 | 45.455 | 1.00 | 38.94 | A |
| ATOM | 656 | CB | SER | A | 140 | 104.690 | 129.641 | 45.509 | 1.00 | 38.80 | A |
| ATOM | 657 | OG | SER | A | 140 | 105.986 | 129.304 | 45.008 | 1.00 | 37.07 | A |
| ATOM | 658 | C | SER | A | 140 | 103.636 | 127.872 | 44.027 | 1.00 | 37.53 | A |
| ATOM | 659 | O | SER | A | 140 | 103.120 | 128.521 | 43.130 | 1.00 | 37.63 | A |
| ATOM | 660 | N | GLY | A | 141 | 104.177 | 126.687 | 43.813 | 1.00 | 35.21 | A |
| ATOM | 661 | CA | GLY | A | 141 | 104.123 | 126.131 | 42.481 | 1.00 | 34.41 | A |
| ATOM | 662 | C | GLY | A | 141 | 105.428 | 126.318 | 41.716 | 1.00 | 34.99 | A |
| ATOM | 663 | O | GLY | A | 141 | 106.065 | 127.381 | 41.772 | 1.00 | 34.08 | A |
| ATOM | 664 | N | ARG | A | 142 | 105.838 | 125.257 | 41.023 | 1.00 | 35.67 | A |
| ATOM | 665 | CA | ARG | A | 142 | 107.040 | 125.262 | 40.200 | 1.00 | 36.89 | A |
| ATOM | 666 | CB | ARG | A | 142 | 106.970 | 124.117 | 39.182 | 1.00 | 38.90 | A |
| ATOM | 667 | CG | ARG | A | 142 | 108.326 | 123.769 | 38.555 | 1.00 | 42.02 | A |
| ATOM | 668 | CD | ARG | A | 142 | 108.115 | 122.996 | 37.273 | 1.00 | 44.92 | A |
| ATOM | 669 | NE | ARG | A | 142 | 109.387 | 122.712 | 36.622 | 1.00 | 48.10 | A |
| ATOM | 670 | CZ | ARG | A | 142 | 110.224 | 121.707 | 36.930 | 1.00 | 50.16 | A |
| ATOM | 671 | NH1 | ARG | A | 142 | 109.948 | 120.825 | 37.900 | 1.00 | 49.37 | A |
| ATOM | 672 | NH2 | ARG | A | 142 | 111.379 | 121.606 | 36.273 | 1.00 | 50.03 | A |
| ATOM | 673 | C | ARG | A | 142 | 107.172 | 126.604 | 39.454 | 1.00 | 36.55 | A |
| ATOM | 674 | O | ARG | A | 142 | 106.187 | 127.169 | 38.949 | 1.00 | 35.70 | A |
| ATOM | 675 | N | GLY | A | 143 | 108.397 | 127.107 | 39.387 | 1.00 | 35.74 | A |
| ATOM | 676 | CA | GLY | A | 143 | 108.644 | 128.377 | 38.738 | 1.00 | 35.11 | A |
| ATOM | 677 | C | GLY | A | 143 | 107.734 | 129.510 | 39.178 | 1.00 | 36.33 | A |
| ATOM | 678 | O | GLY | A | 143 | 107.624 | 130.501 | 38.489 | 1.00 | 38.15 | A |
| ATOM | 679 | N | LYS | A | 144 | 107.083 | 129.423 | 40.320 | 1.00 | 35.60 | A |
| ATOM | 680 | CA | LYS | A | 144 | 106.216 | 130.521 | 40.674 | 1.00 | 35.54 | A |
| ATOM | 681 | CB | LYS | A | 144 | 104.769 | 130.055 | 40.630 | 1.00 | 37.52 | A |
| ATOM | 682 | CG | LYS | A | 144 | 104.286 | 129.818 | 39.226 | 1.00 | 37.93 | A |
| ATOM | 683 | CD | LYS | A | 144 | 102.805 | 129.403 | 39.199 | 1.00 | 38.46 | A |
| ATOM | 684 | CE | LYS | A | 144 | 102.323 | 129.284 | 37.754 | 1.00 | 37.65 | A |
| ATOM | 685 | NZ | LYS | A | 144 | 100.929 | 128.895 | 37.762 | 1.00 | 39.11 | A |
| ATOM | 686 | C | LYS | A | 144 | 106.476 | 131.160 | 42.017 | 1.00 | 35.13 | A |
| ATOM | 687 | O | LYS | A | 144 | 107.067 | 130.564 | 42.906 | 1.00 | 35.94 | A |
| ATOM | 688 | N | SER | A | 145 | 106.039 | 132.391 | 42.189 | 1.00 | 33.52 | A |
| ATOM | 689 | CA | SER | A | 145 | 106.244 | 132.978 | 43.468 | 1.00 | 33.19 | A |
| ATOM | 690 | CB | SER | A | 145 | 107.405 | 133.912 | 43.395 | 1.00 | 34.21 | A |
| ATOM | 691 | OG | SER | A | 145 | 106.889 | 135.092 | 42.877 | 1.00 | 40.10 | A |
| ATOM | 692 | C | SER | A | 145 | 104.979 | 133.652 | 43.997 | 1.00 | 31.94 | A |
| ATOM | 693 | O | SER | A | 145 | 104.068 | 133.992 | 43.259 | 1.00 | 31.77 | A |
| ATOM | 694 | N | PHE | A | 146 | 104.923 | 133.805 | 45.304 | 1.00 | 31.80 | A |
| ATOM | 695 | CA | PHE | A | 146 | 103.754 | 134.365 | 45.952 | 1.00 | 30.69 | A |
| ATOM | 696 | CB | PHE | A | 146 | 103.724 | 133.986 | 47.468 | 1.00 | 30.68 | A |
| ATOM | 697 | CG | PHE | A | 146 | 103.237 | 132.573 | 47.765 | 1.00 | 29.20 | A |
| ATOM | 698 | CD1 | PHE | A | 146 | 103.994 | 131.731 | 48.570 | 1.00 | 27.86 | A |
| ATOM | 699 | CD2 | PHE | A | 146 | 102.042 | 132.081 | 47.209 | 1.00 | 28.48 | A |
| ATOM | 700 | CE1 | PHE | A | 146 | 103.599 | 130.406 | 48.824 | 1.00 | 27.58 | A |
| ATOM | 701 | CE2 | PHE | A | 146 | 101.633 | 130.759 | 47.458 | 1.00 | 28.89 | A |
| ATOM | 702 | CZ | PHE | A | 146 | 102.422 | 129.919 | 48.269 | 1.00 | 28.25 | A |
| ATOM | 703 | C | PHE | A | 146 | 103.730 | 135.841 | 45.850 | 1.00 | 29.94 | A |
| ATOM | 704 | O | PHE | A | 146 | 104.754 | 136.500 | 45.718 | 1.00 | 29.82 | A |
| ATOM | 705 | N | THR | A | 147 | 102.532 | 136.360 | 45.918 | 1.00 | 29.70 | A |
| ATOM | 706 | CA | THR | A | 147 | 102.348 | 137.794 | 45.942 | 1.00 | 30.52 | A |
| ATOM | 707 | CB | THR | A | 147 | 101.225 | 138.174 | 44.990 | 1.00 | 30.37 | A |
| ATOM | 708 | OG1 | THR | A | 147 | 101.749 | 138.122 | 43.663 | 1.00 | 32.55 | A |
| ATOM | 709 | CG2 | THR | A | 147 | 100.689 | 139.564 | 45.271 | 1.00 | 31.36 | A |
| ATOM | 710 | C | THR | A | 147 | 101.941 | 138.028 | 47.396 | 1.00 | 30.64 | A |
| ATOM | 711 | O | THR | A | 147 | 101.037 | 137.341 | 47.903 | 1.00 | 29.60 | A |
| ATOM | 712 | N | LEU | A | 148 | 102.620 | 138.930 | 48.100 | 1.00 | 30.11 | A |
| ATOM | 713 | CA | LEU | A | 148 | 102.234 | 139.131 | 49.478 | 1.00 | 29.69 | A |
| ATOM | 714 | CB | LEU | A | 148 | 103.414 | 139.534 | 50.330 | 1.00 | 28.01 | A |
| ATOM | 715 | CG | LEU | A | 148 | 104.596 | 138.594 | 50.386 | 1.00 | 30.59 | A |
| ATOM | 716 | CD1 | LEU | A | 148 | 105.583 | 139.089 | 51.509 | 1.00 | 27.70 | A |
| ATOM | 717 | CD2 | LEU | A | 148 | 104.091 | 137.153 | 50.647 | 1.00 | 28.77 | A |
| ATOM | 718 | C | LEU | A | 148 | 101.233 | 140.249 | 49.584 | 1.00 | 30.74 | A |
| ATOM | 719 | O | LEU | A | 148 | 101.235 | 141.184 | 48.743 | 1.00 | 31.21 | A |
| ATOM | 720 | N | THR | A | 149 | 100.384 | 140.151 | 50.621 | 1.00 | 29.38 | A |
| ATOM | 721 | CA | THR | A | 149 | 99.488 | 141.239 | 50.967 | 1.00 | 28.23 | A |
| ATOM | 722 | CB | THR | A | 149 | 97.988 | 140.867 | 50.848 | 1.00 | 29.13 | A |
| ATOM | 723 | OG1 | THR | A | 149 | 97.653 | 140.625 | 49.459 | 1.00 | 26.50 | A |
| ATOM | 724 | CG2 | THR | A | 149 | 97.141 | 142.029 | 51.403 | 1.00 | 24.70 | A |
| ATOM | 725 | C | THR | A | 149 | 99.818 | 141.637 | 52.417 | 1.00 | 27.50 | A |
| ATOM | 726 | O | THR | A | 149 | 99.654 | 140.839 | 53.339 | 1.00 | 27.36 | A |
| ATOM | 727 | N | ILE | A | 150 | 100.325 | 142.860 | 52.599 | 1.00 | 26.45 | A |
| ATOM | 728 | CA | ILE | A | 150 | 100.689 | 143.364 | 53.911 | 1.00 | 26.37 | A |
| ATOM | 729 | CB | ILE | A | 150 | 102.059 | 144.119 | 53.875 | 1.00 | 25.38 | A |
| ATOM | 730 | CG2 | ILE | A | 150 | 102.342 | 144.797 | 55.172 | 1.00 | 21.32 | A |
| ATOM | 731 | CG1 | ILE | A | 150 | 103.188 | 143.154 | 53.582 | 1.00 | 22.82 | A |
| ATOM | 732 | CD1 | ILE | A | 150 | 104.401 | 143.921 | 53.185 | 1.00 | 21.25 | A |
| ATOM | 733 | C | ILE | A | 150 | 99.599 | 144.317 | 54.218 | 1.00 | 27.19 | A |
| ATOM | 734 | O | ILE | A | 150 | 99.367 | 145.220 | 53.404 | 1.00 | 27.20 | A |
| ATOM | 735 | N | THR | A | 151 | 98.927 | 144.102 | 55.363 | 1.00 | 27.95 | A |
| ATOM | 736 | CA | THR | A | 151 | 97.810 | 144.947 | 55.809 | 1.00 | 29.63 | A |
| ATOM | 737 | CB | THR | A | 151 | 96.551 | 144.168 | 56.002 | 1.00 | 29.92 | A |
| ATOM | 738 | OG1 | THR | A | 151 | 96.265 | 143.444 | 54.803 | 1.00 | 32.57 | A |
| ATOM | 739 | CG2 | THR | A | 151 | 95.393 | 145.103 | 56.233 | 1.00 | 30.78 | A |
| ATOM | 740 | C | THR | A | 151 | 98.143 | 145.614 | 57.125 | 1.00 | 31.47 | A |
| ATOM | 741 | O | THR | A | 151 | 98.536 | 144.931 | 58.095 | 1.00 | 34.30 | A |
| ATOM | 742 | N | VAL | A | 152 | 98.040 | 146.943 | 57.159 | 1.00 | 31.00 | A |
| ATOM | 743 | CA | VAL | A | 152 | 98.352 | 147.674 | 58.364 | 1.00 | 31.32 | A |
| ATOM | 744 | CB | VAL | A | 152 | 99.379 | 148.774 | 58.088 | 1.00 | 30.52 | A |
| ATOM | 745 | CG1 | VAL | A | 152 | 99.448 | 149.725 | 59.263 | 1.00 | 26.04 | A |
| ATOM | 746 | CG2 | VAL | A | 152 | 100.746 | 148.110 | 57.783 | 1.00 | 25.44 | A |
| ATOM | 747 | C | VAL | A | 152 | 97.008 | 148.202 | 58.836 | 1.00 | 32.99 | A |
| ATOM | 748 | O | VAL | A | 152 | 96.342 | 148.949 | 58.147 | 1.00 | 33.23 | A |
| ATOM | 749 | N | PHE | A | 153 | 96.611 | 147.757 | 60.022 | 1.00 | 33.91 | A |
| ATOM | 750 | CA | PHE | A | 153 | 95.306 | 148.070 | 60.558 | 1.00 | 35.31 | A |
| ATOM | 751 | CB | PHE | A | 153 | 94.858 | 146.895 | 61.450 | 1.00 | 33.39 | A |
| ATOM | 752 | CG | PHE | A | 153 | 94.585 | 145.629 | 60.667 | 1.00 | 33.50 | A |
| ATOM | 753 | CD1 | PHE | A | 153 | 95.625 | 144.740 | 60.349 | 1.00 | 32.98 | A |
| ATOM | 754 | CD2 | PHE | A | 153 | 93.305 | 145.340 | 60.202 | 1.00 | 30.84 | A |
| ATOM | 755 | CE1 | PHE | A | 153 | 95.377 | 143.582 | 59.579 | 1.00 | 32.22 | A |
| ATOM | 756 | CE2 | PHE | A | 153 | 93.067 | 144.201 | 59.446 | 1.00 | 30.16 | A |
| ATOM | 757 | CZ | PHE | A | 153 | 94.095 | 143.320 | 59.133 | 1.00 | 30.57 | A |
| ATOM | 758 | C | PHE | A | 153 | 95.042 | 149.409 | 61.234 | 1.00 | 36.75 | A |
| ATOM | 759 | O | PHE | A | 153 | 94.884 | 149.494 | 62.438 | 1.00 | 35.87 | A |
| ATOM | 760 | N | THR | A | 154 | 94.989 | 150.452 | 60.420 | 1.00 | 39.13 | A |
| ATOM | 761 | CA | THR | A | 154 | 94.674 | 151.809 | 60.871 | 1.00 | 41.41 | A |
| ATOM | 762 | CB | THR | A | 154 | 95.522 | 152.805 | 60.080 | 1.00 | 40.61 | A |
| ATOM | 763 | OG1 | THR | A | 154 | 95.568 | 152.400 | 58.706 | 1.00 | 39.62 | A |
| ATOM | 764 | CG2 | THR | A | 154 | 96.933 | 152.821 | 60.594 | 1.00 | 39.01 | A |
| ATOM | 765 | C | THR | A | 154 | 93.212 | 151.885 | 60.402 | 1.00 | 44.12 | A |
| ATOM | 766 | O | THR | A | 154 | 92.730 | 150.885 | 59.846 | 1.00 | 47.03 | A |
| ATOM | 767 | N | ASN | A | 155 | 92.496 | 152.996 | 60.608 | 1.00 | 45.71 | A |
| ATOM | 768 | CA | ASN | A | 155 | 91.111 | 153.111 | 60.117 | 1.00 | 46.43 | A |
| ATOM | 769 | CB | ASN | A | 155 | 90.183 | 153.494 | 61.208 | 1.00 | 49.54 | A |
| ATOM | 770 | CG | ASN | A | 155 | 90.342 | 152.644 | 62.358 | 1.00 | 54.70 | A |
| ATOM | 771 | OD1 | ASN | A | 155 | 90.049 | 151.441 | 62.285 | 1.00 | 58.51 | A |
| ATOM | 772 | ND2 | ASN | A | 155 | 90.852 | 153.215 | 63.462 | 1.00 | 58.02 | A |
| ATOM | 773 | C | ASN | A | 155 | 91.006 | 154.199 | 59.083 | 1.00 | 46.07 | A |
| ATOM | 774 | O | ASN | A | 155 | 91.073 | 155.383 | 59.423 | 1.00 | 48.00 | A |
| ATOM | 775 | N | CPR | A | 156 | 90.821 | 153.830 | 57.815 | 1.00 | 44.15 | A |
| ATOM | 776 | CD | CPR | A | 156 | 90.677 | 154.737 | 56.669 | 1.00 | 43.51 | A |
| ATOM | 777 | CA | CPR | A | 156 | 90.719 | 152.432 | 57.382 | 1.00 | 42.96 | A |
| ATOM | 778 | CB | CPR | A | 156 | 90.063 | 152.538 | 56.008 | 1.00 | 41.80 | A |
| ATOM | 779 | CG | CPR | A | 156 | 90.693 | 153.760 | 55.461 | 1.00 | 43.38 | A |
| ATOM | 780 | C | CPR | A | 156 | 92.096 | 151.760 | 57.297 | 1.00 | 41.23 | A |
| ATOM | 781 | O | CPR | A | 156 | 93.135 | 152.360 | 57.539 | 1.00 | 40.92 | A |
| ATOM | 782 | N | PRO | A | 157 | 92.098 | 150.483 | 56.978 | 1.00 | 40.21 | A |
| ATOM | 783 | CD | PRO | A | 157 | 90.933 | 149.587 | 56.850 | 1.00 | 38.86 | A |
| ATOM | 784 | CA | PRO | A | 157 | 93.368 | 149.766 | 56.867 | 1.00 | 36.73 | A |
| ATOM | 785 | CB | PRO | A | 157 | 92.922 | 148.316 | 56.689 | 1.00 | 38.77 | A |
| ATOM | 786 | CG | PRO | A | 157 | 91.501 | 148.298 | 57.315 | 1.00 | 39.41 | A |
| ATOM | 787 | C | PRO | A | 157 | 94.157 | 150.260 | 55.635 | 1.00 | 37.57 | A |
| ATOM | 788 | O | PRO | A | 157 | 93.581 | 150.750 | 54.691 | 1.00 | 37.58 | A |
| ATOM | 789 | N | GLN | A | 158 | 95.479 | 150.157 | 55.669 | 1.00 | 36.77 | A |
| ATOM | 790 | CA | GLN | A | 158 | 96.311 | 150.500 | 54.537 | 1.00 | 34.80 | A |
| ATOM | 791 | CB | GLN | A | 158 | 97.459 | 151.418 | 54.940 | 1.00 | 35.03 | A |
| ATOM | 792 | CG | GLN | A | 158 | 96.982 | 152.756 | 55.402 | 1.00 | 36.72 | A |
| ATOM | 793 | CD | GLN | A | 158 | 98.109 | 153.683 | 55.786 | 1.00 | 36.96 | A |
| ATOM | 794 | OE1 | GLN | A | 158 | 98.854 | 154.136 | 54.943 | 1.00 | 37.08 | A |
| ATOM | 795 | NE2 | GLN | A | 158 | 98.219 | 153.983 | 57.075 | 1.00 | 38.29 | A |
| ATOM | 796 | C | GLN | A | 158 | 96.854 | 149.144 | 54.109 | 1.00 | 34.24 | A |
| ATOM | 797 | O | GLN | A | 158 | 97.280 | 148.341 | 54.948 | 1.00 | 34.53 | A |
| ATOM | 798 | N | VAL | A | 159 | 96.837 | 148.892 | 52.810 | 1.00 | 32.24 | A |
| ATOM | 799 | CA | VAL | A | 159 | 97.308 | 147.639 | 52.273 | 1.00 | 30.89 | A |
| ATOM | 800 | CB | VAL | A | 159 | 96.123 | 146.913 | 51.628 | 1.00 | 31.78 | A |
| ATOM | 801 | CG1 | VAL | A | 159 | 96.534 | 145.559 | 51.133 | 1.00 | 30.76 | A |
| ATOM | 802 | CG2 | VAL | A | 159 | 95.017 | 146.784 | 52.652 | 1.00 | 29.08 | A |
| ATOM | 803 | C | VAL | A | 159 | 98.432 | 147.827 | 51.257 | 1.00 | 31.13 | A |
| ATOM | 804 | O | VAL | A | 159 | 98.429 | 148.740 | 50.454 | 1.00 | 31.30 | A |
| ATOM | 805 | N | ALA | A | 160 | 99.414 | 146.953 | 51.302 | 1.00 | 31.84 | A |
| ATOM | 806 | CA | ALA | A | 160 | 100.550 | 147.034 | 50.388 | 1.00 | 32.70 | A |
| ATOM | 807 | CB | ALA | A | 160 | 101.821 | 147.630 | 51.099 | 1.00 | 30.99 | A |
| ATOM | 808 | C | ALA | A | 160 | 100.879 | 145.640 | 49.877 | 1.00 | 34.13 | A |
| ATOM | 809 | O | ALA | A | 160 | 101.065 | 144.677 | 50.665 | 1.00 | 35.25 | A |
| ATOM | 810 | N | THR | A | 161 | 100.979 | 145.511 | 48.563 | 1.00 | 33.85 | A |
| ATOM | 811 | CA | THR | A | 161 | 101.308 | 144.217 | 48.032 | 1.00 | 33.75 | A |
| ATOM | 812 | CB | THR | A | 161 | 100.426 | 143.891 | 46.807 | 1.00 | 32.71 | A |
| ATOM | 813 | OG1 | THR | A | 161 | 100.516 | 144.938 | 45.661 | 1.00 | 32.67 | A |
| ATOM | 814 | CG2 | THR | A | 161 | 98.983 | 143.785 | 47.182 | 1.00 | 32.42 | A |
| ATOM | 815 | C | THR | A | 161 | 102.802 | 144.227 | 47.674 | 1.00 | 34.84 | A |
| ATOM | 816 | O | THR | A | 161 | 103.439 | 145.282 | 47.587 | 1.00 | 34.43 | A |
| ATOM | 817 | N | TYR | A | 162 | 103.345 | 143.038 | 47.528 | 1.00 | 35.00 | A |
| ATOM | 818 | CA | TYR | A | 162 | 104.726 | 142.834 | 47.173 | 1.00 | 36.92 | A |
| ATOM | 819 | CB | TYR | A | 162 | 105.491 | 142.466 | 48.439 | 1.00 | 38.63 | A |
| ATOM | 820 | CG | TYR | A | 162 | 106.966 | 142.170 | 48.313 | 1.00 | 41.23 | A |
| ATOM | 821 | CD1 | TYR | A | 162 | 107.649 | 141.664 | 49.415 | 1.00 | 42.60 | A |
| ATOM | 822 | CE1 | TYR | A | 162 | 108.984 | 141.340 | 49.355 | 1.00 | 46.22 | A |
| ATOM | 823 | CD2 | TYR | A | 162 | 107.656 | 142.347 | 47.126 | 1.00 | 42.07 | A |
| ATOM | 824 | CE2 | TYR | A | 162 | 109.009 | 142.025 | 47.027 | 1.00 | 45.37 | A |
| ATOM | 825 | CZ | TYR | A | 162 | 109.687 | 141.518 | 48.156 | 1.00 | 48.16 | A |
| ATOM | 826 | OH | TYR | A | 162 | 111.062 | 141.192 | 48.136 | 1.00 | 50.51 | A |
| ATOM | 827 | C | TYR | A | 162 | 104.569 | 141.653 | 46.185 | 1.00 | 37.28 | A |
| ATOM | 828 | O | TYR | A | 162 | 104.286 | 140.516 | 46.571 | 1.00 | 37.14 | A |
| ATOM | 829 | N | HIS | A | 163 | 104.666 | 141.943 | 44.892 | 1.00 | 37.89 | A |
| ATOM | 830 | CA | HIS | A | 163 | 104.512 | 140.903 | 43.885 | 1.00 | 38.08 | A |
| ATOM | 831 | CB | HIS | A | 163 | 104.054 | 141.520 | 42.550 | 1.00 | 38.31 | A |
| ATOM | 832 | CG | HIS | A | 163 | 102.575 | 141.797 | 42.494 | 1.00 | 39.32 | A |
| ATOM | 833 | CD2 | HIS | A | 163 | 101.556 | 141.061 | 41.997 | 1.00 | 37.89 | A |
| ATOM | 834 | ND1 | HIS | A | 163 | 101.988 | 142.906 | 43.076 | 1.00 | 40.25 | A |
| ATOM | 835 | CE1 | HIS | A | 163 | 100.679 | 142.841 | 42.940 | 1.00 | 38.19 | A |
| ATOM | 836 | NE2 | HIS | A | 163 | 100.394 | 141.730 | 42.288 | 1.00 | 39.38 | A |
| ATOM | 837 | C | HIS | A | 163 | 105.772 | 140.060 | 43.676 | 1.00 | 38.59 | A |
| ATOM | 838 | O | HIS | A | 163 | 106.918 | 140.554 | 43.820 | 1.00 | 38.21 | A |
| ATOM | 839 | N | ARG | A | 164 | 105.564 | 138.775 | 43.377 | 1.00 | 38.49 | A |
| ATOM | 840 | CA | ARG | A | 164 | 106.692 | 137.885 | 43.105 | 1.00 | 39.06 | A |
| ATOM | 841 | CB | ARG | A | 164 | 107.335 | 138.249 | 41.747 | 1.00 | 39.78 | A |
| ATOM | 842 | CG | ARG | A | 164 | 106.456 | 137.916 | 40.578 | 1.00 | 43.72 | A |
| ATOM | 843 | CD | ARG | A | 164 | 106.940 | 138.529 | 39.258 | 1.00 | 50.23 | A |
| ATOM | 844 | NE | ARG | A | 164 | 106.060 | 139.658 | 38.891 | 1.00 | 57.59 | A |
| ATOM | 845 | CZ | ARG | A | 164 | 106.351 | 140.972 | 38.962 | 1.00 | 59.53 | A |
| ATOM | 846 | NH1 | ARG | A | 164 | 107.562 | 141.412 | 39.379 | 1.00 | 60.94 | A |
| ATOM | 847 | NH2 | ARG | A | 164 | 105.378 | 141.857 | 38.666 | 1.00 | 60.09 | A |
| ATOM | 848 | C | ARG | A | 164 | 107.718 | 138.026 | 44.208 | 1.00 | 37.85 | A |
| ATOM | 849 | O | ARG | A | 164 | 108.919 | 138.126 | 43.964 | 1.00 | 38.47 | A |
| ATOM | 850 | N | ALA | A | 165 | 107.241 | 138.017 | 45.436 | 1.00 | 37.01 | A |
| ATOM | 851 | CA | ALA | A | 165 | 108.136 | 138.184 | 46.585 | 1.00 | 36.10 | A |
| ATOM | 852 | CB | ALA | A | 165 | 107.330 | 138.653 | 47.819 | 1.00 | 34.36 | A |
| ATOM | 853 | C | ALA | A | 165 | 108.925 | 136.948 | 46.962 | 1.00 | 35.11 | A |
| ATOM | 854 | O | ALA | A | 165 | 110.032 | 137.072 | 47.424 | 1.00 | 36.59 | A |
| ATOM | 855 | N | ILE | A | 166 | 108.357 | 135.761 | 46.795 | 1.00 | 33.30 | A |
| ATOM | 856 | CA | ILE | A | 166 | 109.077 | 134.585 | 47.208 | 1.00 | 31.23 | A |
| ATOM | 857 | CB | ILE | A | 166 | 109.105 | 134.525 | 48.715 | 1.00 | 31.57 | A |
| ATOM | 858 | CG2 | ILE | A | 166 | 107.653 | 134.479 | 49.291 | 1.00 | 30.92 | A |
| ATOM | 859 | CG1 | ILE | A | 166 | 109.848 | 133.275 | 49.171 | 1.00 | 31.59 | A |
| ATOM | 860 | CD1 | ILE | A | 166 | 109.961 | 133.193 | 50.695 | 1.00 | 31.21 | A |
| ATOM | 861 | C | ILE | A | 166 | 108.453 | 133.315 | 46.697 | 1.00 | 30.25 | A |
| ATOM | 862 | O | ILE | A | 166 | 107.240 | 133.215 | 46.638 | 1.00 | 29.37 | A |
| ATOM | 863 | N | LYS | A | 167 | 109.289 | 132.369 | 46.287 | 1.00 | 30.24 | A |
| ATOM | 864 | CA | LYS | A | 167 | 108.809 | 131.062 | 45.832 | 1.00 | 31.48 | A |
| ATOM | 865 | CB | LYS | A | 167 | 109.576 | 130.585 | 44.602 | 1.00 | 32.30 | A |
| ATOM | 866 | CG | LYS | A | 167 | 109.329 | 129.145 | 44.255 | 1.00 | 32.37 | A |
| ATOM | 867 | CD | LYS | A | 167 | 109.962 | 128.788 | 42.907 | 1.00 | 30.90 | A |
| ATOM | 868 | CE | LYS | A | 167 | 111.481 | 128.740 | 42.961 | 1.00 | 30.89 | A |
| ATOM | 869 | NZ | LYS | A | 167 | 112.071 | 128.202 | 41.659 | 1.00 | 31.69 | A |
| ATOM | 870 | C | LYS | A | 167 | 109.037 | 130.084 | 47.003 | 1.00 | 32.11 | A |
| ATOM | 871 | O | LYS | A | 167 | 110.138 | 130.049 | 47.624 | 1.00 | 32.76 | A |
| ATOM | 872 | N | ILE | A | 168 | 107.989 | 129.336 | 47.328 | 1.00 | 30.71 | A |
| ATOM | 873 | CA | ILE | A | 168 | 108.065 | 128.382 | 48.401 | 1.00 | 31.36 | A |
| ATOM | 874 | CB | ILE | A | 168 | 106.998 | 128.679 | 49.438 | 1.00 | 30.22 | A |
| ATOM | 875 | CG2 | ILE | A | 168 | 106.946 | 127.502 | 50.433 | 1.00 | 27.64 | A |
| ATOM | 876 | CG1 | ILE | A | 168 | 107.278 | 130.025 | 50.071 | 1.00 | 28.53 | A |
| ATOM | 877 | CD1 | ILE | A | 168 | 108.617 | 130.055 | 50.876 | 1.00 | 28.88 | A |
| ATOM | 878 | C | ILE | A | 168 | 107.881 | 126.949 | 47.890 | 1.00 | 31.79 | A |
| ATOM | 879 | O | ILE | A | 168 | 106.859 | 126.611 | 47.325 | 1.00 | 32.83 | A |
| ATOM | 880 | N | THR | A | 169 | 108.856 | 126.102 | 48.116 | 1.00 | 31.80 | A |
| ATOM | 881 | CA | THR | A | 169 | 108.785 | 124.725 | 47.622 | 1.00 | 31.71 | A |
| ATOM | 882 | CB | THR | A | 169 | 109.858 | 124.426 | 46.581 | 1.00 | 29.64 | A |
| ATOM | 883 | OG1 | THR | A | 169 | 111.158 | 124.595 | 47.197 | 1.00 | 29.76 | A |
| ATOM | 884 | CG2 | THR | A | 169 | 109.715 | 125.369 | 45.402 | 1.00 | 28.25 | A |
| ATOM | 885 | C | THR | A | 169 | 109.091 | 123.835 | 48.795 | 1.00 | 33.41 | A |
| ATOM | 886 | O | THR | A | 169 | 109.666 | 124.282 | 49.761 | 1.00 | 33.19 | A |
| ATOM | 887 | N | VAL | A | 170 | 108.767 | 122.555 | 48.640 | 1.00 | 35.11 | A |
| ATOM | 888 | CA | VAL | A | 170 | 108.899 | 121.545 | 49.653 | 1.00 | 34.80 | A |
| ATOM | 889 | CB | VAL | A | 170 | 108.227 | 120.286 | 49.111 | 1.00 | 36.72 | A |
| ATOM | 890 | CG1 | VAL | A | 170 | 108.949 | 119.053 | 49.556 | 1.00 | 37.68 | A |
| ATOM | 891 | CG2 | VAL | A | 170 | 106.754 | 120.260 | 49.574 | 1.00 | 36.35 | A |
| ATOM | 892 | C | VAL | A | 170 | 110.316 | 121.335 | 50.076 | 1.00 | 34.79 | A |
| ATOM | 893 | O | VAL | A | 170 | 110.604 | 121.055 | 51.240 | 1.00 | 38.27 | A |
| ATOM | 894 | N | ASP | A | 171 | 111.228 | 121.520 | 49.160 | 1.00 | 34.22 | A |
| ATOM | 895 | CA | ASP | A | 171 | 112.624 | 121.340 | 49.461 | 1.00 | 34.75 | A |
| ATOM | 896 | CB | ASP | A | 171 | 113.290 | 120.623 | 48.303 | 1.00 | 34.45 | A |
| ATOM | 897 | CG | ASP | A | 171 | 112.977 | 119.113 | 48.280 | 1.00 | 36.90 | A |
| ATOM | 898 | OD1 | ASP | A | 171 | 112.153 | 118.616 | 49.107 | 1.00 | 34.28 | A |
| ATOM | 899 | OD2 | ASP | A | 171 | 113.575 | 118.419 | 47.414 | 1.00 | 38.84 | A |
| ATOM | 900 | C | ASP | A | 171 | 113.384 | 122.609 | 49.719 | 1.00 | 35.86 | A |
| ATOM | 901 | O | ASP | A | 171 | 114.331 | 122.633 | 50.494 | 1.00 | 36.44 | A |
| ATOM | 902 | N | GLY | A | 172 | 112.980 | 123.675 | 49.055 | 1.00 | 36.56 | A |
| ATOM | 903 | CA | GLY | A | 172 | 113.713 | 124.902 | 49.202 | 1.00 | 37.50 | A |
| ATOM | 904 | C | GLY | A | 172 | 114.922 | 124.802 | 48.285 | 1.00 | 38.31 | A |
| ATOM | 905 | O | GLY | A | 172 | 115.138 | 123.816 | 47.624 | 1.00 | 36.60 | A |
| ATOM | 906 | N | PRO | A | 173 | 115.736 | 125.842 | 48.219 | 1.00 | 40.22 | A |
| ATOM | 907 | CD | PRO | A | 173 | 115.553 | 127.128 | 48.907 | 1.00 | 39.50 | A |
| ATOM | 908 | CA | PRO | A | 173 | 116.927 | 125.864 | 47.373 | 1.00 | 41.64 | A |
| ATOM | 909 | CB | PRO | A | 173 | 117.652 | 127.103 | 47.865 | 1.00 | 39.62 | A |
| ATOM | 910 | CG | PRO | A | 173 | 116.558 | 128.000 | 48.197 | 1.00 | 40.22 | A |
| ATOM | 911 | C | PRO | A | 173 | 117.759 | 124.622 | 47.573 | 1.00 | 44.53 | A |
| ATOM | 912 | O | PRO | A | 173 | 117.961 | 124.204 | 48.730 | 1.00 | 45.57 | A |
| ATOM | 913 | N | ARG | A | 174 | 118.240 | 124.041 | 46.467 | 1.00 | 47.04 | A |
| ATOM | 914 | CA | ARG | A | 174 | 119.103 | 122.856 | 46.515 | 1.00 | 51.03 | A |
| ATOM | 915 | CB | ARG | A | 174 | 118.296 | 121.547 | 46.431 | 1.00 | 49.51 | A |
| ATOM | 916 | CG | ARG | A | 174 | 117.986 | 120.959 | 47.805 | 1.00 | 47.33 | A |
| ATOM | 917 | CD | ARG | A | 174 | 117.162 | 119.702 | 47.711 | 1.00 | 47.24 | A |
| ATOM | 918 | NE | ARG | A | 174 | 117.932 | 118.478 | 47.516 | 1.00 | 44.74 | A |
| ATOM | 919 | CZ | ARG | A | 174 | 117.353 | 117.297 | 47.496 | 1.00 | 45.23 | A |
| ATOM | 920 | NH1 | ARG | A | 174 | 116.043 | 117.214 | 47.653 | 1.00 | 45.98 | A |
| ATOM | 921 | NH2 | ARG | A | 174 | 118.066 | 116.212 | 47.345 | 1.00 | 45.67 | A |
| ATOM | 922 | C | ARG | A | 174 | 120.198 | 122.830 | 45.476 | 1.00 | 53.96 | A |
| ATOM | 923 | O | ARG | A | 174 | 119.990 | 123.194 | 44.334 | 1.00 | 53.76 | A |
| ATOM | 924 | N | GLU | A | 175 | 121.383 | 122.387 | 45.890 | 1.00 | 58.77 | A |
| ATOM | 925 | CA | GLU | A | 175 | 122.526 | 122.286 | 44.973 | 1.00 | 63.27 | A |
| ATOM | 926 | CB | GLU | A | 175 | 123.771 | 121.929 | 45.765 | 1.00 | 65.78 | A |
| ATOM | 927 | CG | GLU | A | 175 | 125.058 | 122.405 | 45.116 | 1.00 | 70.55 | A |
| ATOM | 928 | CD | GLU | A | 175 | 125.143 | 123.946 | 44.972 | 1.00 | 72.57 | A |
| ATOM | 929 | OE1 | GLU | A | 175 | 126.070 | 124.440 | 44.270 | 1.00 | 74.57 | A |
| ATOM | 930 | OE2 | GLU | A | 175 | 124.291 | 124.660 | 45.556 | 1.00 | 73.07 | A |
| ATOM | 931 | C | GLU | A | 175 | 122.273 | 121.210 | 43.899 | 1.00 | 64.46 | A |
| ATOM | 932 | O | GLU | A | 175 | 121.814 | 120.106 | 44.208 | 1.00 | 65.76 | A |
| ATOM | 933 | N | PRO | A | 176 | 122.576 | 121.509 | 42.632 | 1.00 | 65.46 | A |
| ATOM | 934 | CD | PRO | A | 176 | 123.441 | 122.624 | 42.213 | 1.00 | 66.09 | A |
| ATOM | 935 | CA | PRO | A | 176 | 122.372 | 120.560 | 41.520 | 1.00 | 66.00 | A |
| ATOM | 936 | CB | PRO | A | 176 | 123.023 | 121.269 | 40.332 | 1.00 | 65.28 | A |
| ATOM | 937 | CG | PRO | A | 176 | 124.103 | 122.052 | 40.967 | 1.00 | 65.52 | A |
| ATOM | 938 | C | PRO | A | 176 | 123.004 | 119.211 | 41.821 | 1.00 | 66.72 | A |
| ATOM | 939 | O | PRO | A | 176 | 123.952 | 119.148 | 42.630 | 1.00 | 66.79 | A |
| ATOM | 940 | N | ARG | A | 177 | 122.495 | 118.137 | 41.197 | 1.00 | 67.41 | A |
| ATOM | 941 | CA | ARG | A | 177 | 123.048 | 116.812 | 41.497 | 1.00 | 68.09 | A |
| ATOM | 942 | CB | ARG | A | 177 | 122.223 | 116.165 | 42.645 | 1.00 | 67.68 | A |
| ATOM | 943 | CG | ARG | A | 177 | 120.683 | 116.344 | 42.720 | 1.00 | 63.90 | A |
| ATOM | 944 | CD | ARG | A | 177 | 120.122 | 115.236 | 43.682 | 1.00 | 61.55 | A |
| ATOM | 945 | NE | ARG | A | 177 | 118.747 | 115.430 | 44.084 | 1.00 | 57.92 | A |
| ATOM | 946 | CZ | ARG | A | 177 | 117.921 | 114.446 | 44.416 | 1.00 | 57.17 | A |
| ATOM | 947 | NH1 | ARG | A | 177 | 118.314 | 113.188 | 44.392 | 1.00 | 56.04 | A |
| ATOM | 948 | NH2 | ARG | A | 177 | 116.686 | 114.731 | 44.780 | 1.00 | 57.03 | A |
| ATOM | 949 | C | ARG | A | 177 | 123.392 | 115.738 | 40.432 | 1.00 | 68.08 | A |
| ATOM | 950 | O | ARG | A | 177 | 122.673 | 115.507 | 39.460 | 1.00 | 69.18 | A |
| ATOM | 951 | N | ALA | A | 178 | 125.380 | 113.657 | 39.138 | 0.00 | 77.86 | A |
| ATOM | 952 | CA | ALA | A | 178 | 124.466 | 112.919 | 38.277 | 0.00 | 77.84 | A |
| ATOM | 953 | CB | ALA | A | 178 | 123.178 | 112.603 | 39.030 | 0.00 | 77.87 | A |
| ATOM | 954 | C | ALA | A | 178 | 124.155 | 113.706 | 37.006 | 0.00 | 77.83 | A |
| ATOM | 955 | O | ALA | A | 178 | 124.351 | 113.133 | 35.912 | 0.00 | 77.83 | A |
| ATOM | 956 | OXT | ALA | A | 178 | 123.719 | 114.881 | 37.118 | 1.00 | 77.71 | A |
| ATOM | 957 | CB | PRO | B | 2 | 102.770 | 160.149 | 51.421 | 1.00 | 33.13 | B |
| ATOM | 958 | CG | PRO | B | 2 | 103.695 | 159.663 | 50.327 | 1.00 | 32.43 | B |
| ATOM | 959 | C | PRO | B | 2 | 101.751 | 157.990 | 52.154 | 1.00 | 34.71 | B |
| ATOM | 960 | O | PRO | B | 2 | 101.939 | 157.013 | 51.434 | 1.00 | 34.30 | B |
| ATOM | 961 | N | PRO | B | 2 | 104.183 | 158.356 | 52.193 | 1.00 | 33.17 | B |
| ATOM | 962 | CD | PRO | B | 2 | 104.835 | 158.959 | 51.020 | 1.00 | 31.40 | B |
| ATOM | 963 | CA | PRO | B | 2 | 102.858 | 158.994 | 52.410 | 1.00 | 34.12 | B |
| ATOM | 964 | N | ARG | B | 3 | 100.588 | 158.224 | 52.738 | 1.00 | 35.57 | B |
| ATOM | 965 | CA | ARG | B | 3 | 99.469 | 157.331 | 52.541 | 1.00 | 34.77 | B |
| ATOM | 966 | CB | ARG | B | 3 | 98.281 | 157.829 | 53.338 | 1.00 | 37.28 | B |
| ATOM | 967 | CG | ARG | B | 3 | 98.587 | 158.376 | 54.705 | 1.00 | 41.02 | B |
| ATOM | 968 | CD | ARG | B | 3 | 97.276 | 158.817 | 55.351 | 1.00 | 44.18 | B |
| ATOM | 969 | NE | ARG | B | 3 | 96.726 | 157.711 | 56.129 | 1.00 | 48.71 | B |
| ATOM | 970 | CZ | ARG | B | 3 | 95.457 | 157.336 | 56.153 | 1.00 | 47.47 | B |
| ATOM | 971 | NH1 | ARG | B | 3 | 94.561 | 157.974 | 55.429 | 1.00 | 49.05 | B |
| ATOM | 972 | NH2 | ARG | B | 3 | 95.099 | 156.307 | 56.905 | 1.00 | 48.21 | B |
| ATOM | 973 | C | ARG | B | 3 | 99.024 | 157.087 | 51.077 | 1.00 | 33.31 | B |
| ATOM | 974 | O | ARG | B | 3 | 98.705 | 155.951 | 50.726 | 1.00 | 32.67 | B |
| ATOM | 975 | N | VAL | B | 4 | 98.993 | 158.099 | 50.223 | 1.00 | 32.81 | B |
| ATOM | 976 | CA | VAL | B | 4 | 98.531 | 157.846 | 48.855 | 1.00 | 34.28 | B |
| ATOM | 977 | CB | VAL | B | 4 | 97.223 | 158.541 | 48.576 | 1.00 | 33.86 | B |
| ATOM | 978 | CG1 | VAL | B | 4 | 96.058 | 157.851 | 49.288 | 1.00 | 32.51 | B |
| ATOM | 979 | CG2 | VAL | B | 4 | 97.368 | 159.959 | 49.040 | 1.00 | 34.55 | B |
| ATOM | 980 | C | VAL | B | 4 | 99.514 | 158.338 | 47.810 | 1.00 | 35.83 | B |
| ATOM | 981 | O | VAL | B | 4 | 100.474 | 159.017 | 48.139 | 1.00 | 37.05 | B |
| ATOM | 982 | N | VAL | B | 5 | 99.307 | 157.977 | 46.551 | 1.00 | 36.26 | B |
| ATOM | 983 | CA | VAL | B | 5 | 100.175 | 158.463 | 45.501 | 1.00 | 36.94 | B |
| ATOM | 984 | CB | VAL | B | 5 | 100.158 | 157.556 | 44.305 | 1.00 | 36.12 | B |
| ATOM | 985 | CG1 | VAL | B | 5 | 100.632 | 156.188 | 44.717 | 1.00 | 35.53 | B |
| ATOM | 986 | CG2 | VAL | B | 5 | 98.719 | 157.467 | 43.716 | 1.00 | 35.15 | B |
| ATOM | 987 | C | VAL | B | 5 | 99.564 | 159.781 | 45.084 | 1.00 | 40.09 | B |
| ATOM | 988 | O | VAL | B | 5 | 98.399 | 160.079 | 45.409 | 1.00 | 41.73 | B |
| ATOM | 989 | N | PRO | B | 6 | 100.308 | 160.600 | 44.330 | 1.00 | 42.18 | B |
| ATOM | 990 | CD | PRO | B | 6 | 101.739 | 160.576 | 44.005 | 1.00 | 40.28 | B |
| ATOM | 991 | CA | PRO | B | 6 | 99.691 | 161.874 | 43.940 | 1.00 | 42.53 | B |
| ATOM | 992 | CB | PRO | B | 6 | 100.868 | 162.704 | 43.483 | 1.00 | 41.16 | B |
| ATOM | 993 | CG | PRO | B | 6 | 101.825 | 161.709 | 43.059 | 1.00 | 41.38 | B |
| ATOM | 994 | C | PRO | B | 6 | 98.549 | 161.854 | 42.942 | 1.00 | 43.01 | B |
| ATOM | 995 | O | PRO | B | 6 | 97.845 | 162.842 | 42.844 | 1.00 | 45.46 | B |
| ATOM | 996 | N | ASP | B | 7 | 98.359 | 160.807 | 42.167 | 1.00 | 43.15 | B |
| ATOM | 997 | CA | ASP | B | 7 | 97.173 | 160.799 | 41.321 | 1.00 | 44.35 | B |
| ATOM | 998 | CB | ASP | B | 7 | 97.490 | 161.146 | 39.850 | 1.00 | 47.67 | B |
| ATOM | 999 | CG | ASP | B | 7 | 96.208 | 161.127 | 38.929 | 1.00 | 51.36 | B |
| ATOM | 1000 | OD1 | ASP | B | 7 | 95.067 | 161.188 | 39.455 | 1.00 | 53.98 | B |
| ATOM | 1001 | OD2 | ASP | B | 7 | 96.326 | 161.055 | 37.674 | 1.00 | 53.25 | B |
| ATOM | 1002 | C | ASP | B | 7 | 96.510 | 159.411 | 41.461 | 1.00 | 43.89 | B |
| ATOM | 1003 | O | ASP | B | 7 | 96.809 | 158.460 | 40.714 | 1.00 | 44.12 | B |
| ATOM | 1004 | N | GLN | B | 8 | 95.623 | 159.298 | 42.450 | 1.00 | 42.65 | B |
| ATOM | 1005 | CA | GLN | B | 8 | 94.949 | 158.042 | 42.740 | 1.00 | 41.85 | B |
| ATOM | 1006 | CB | GLN | B | 8 | 94.073 | 158.193 | 43.975 | 1.00 | 40.88 | B |
| ATOM | 1007 | CG | GLN | B | 8 | 94.909 | 158.539 | 45.180 | 1.00 | 40.17 | B |
| ATOM | 1008 | CD | GLN | B | 8 | 94.137 | 158.586 | 46.451 | 1.00 | 40.71 | B |
| ATOM | 1009 | OE1 | GLN | B | 8 | 93.483 | 157.631 | 46.834 | 1.00 | 42.29 | B |
| ATOM | 1010 | NE2 | GLN | B | 8 | 94.239 | 159.687 | 47.146 | 1.00 | 40.55 | B |
| ATOM | 1011 | C | GLN | B | 8 | 94.150 | 157.522 | 41.594 | 1.00 | 43.00 | B |
| ATOM | 1012 | O | GLN | B | 8 | 94.427 | 156.430 | 41.083 | 1.00 | 43.00 | B |
| ATOM | 1013 | N | ARG | B | 9 | 93.176 | 158.309 | 41.156 | 1.00 | 44.11 | B |
| ATOM | 1014 | CA | ARG | B | 9 | 92.371 | 157.877 | 40.048 | 1.00 | 44.59 | B |
| ATOM | 1015 | CB | ARG | B | 9 | 91.429 | 158.989 | 39.584 | 1.00 | 47.07 | B |
| ATOM | 1016 | CG | ARG | B | 9 | 90.687 | 158.619 | 38.299 | 1.00 | 50.23 | B |
| ATOM | 1017 | CD | ARG | B | 9 | 89.441 | 159.465 | 38.019 | 1.00 | 52.66 | B |
| ATOM | 1018 | NE | ARG | B | 9 | 88.188 | 158.902 | 38.527 | 1.00 | 54.08 | B |
| ATOM | 1019 | CZ | ARG | B | 9 | 87.444 | 159.528 | 39.442 | 1.00 | 57.02 | B |
| ATOM | 1020 | NH1 | ARG | B | 9 | 87.849 | 160.716 | 39.926 | 1.00 | 58.17 | B |
| ATOM | 1021 | NH2 | ARG | B | 9 | 86.302 | 158.987 | 39.885 | 1.00 | 57.97 | B |
| ATOM | 1022 | C | ARG | B | 9 | 93.195 | 157.362 | 38.866 | 1.00 | 44.17 | B |
| ATOM | 1023 | O | ARG | B | 9 | 92.881 | 156.290 | 38.299 | 1.00 | 43.42 | B |
| ATOM | 1024 | N | SER | B | 10 | 94.259 | 158.063 | 38.478 | 1.00 | 44.06 | B |
| ATOM | 1025 | CA | SER | B | 10 | 94.993 | 157.558 | 37.319 | 1.00 | 44.47 | B |
| ATOM | 1026 | CB | SER | B | 10 | 96.034 | 158.562 | 36.882 | 1.00 | 44.76 | B |
| ATOM | 1027 | OG | SER | B | 10 | 96.631 | 158.126 | 35.669 | 1.00 | 50.56 | B |
| ATOM | 1028 | C | SER | B | 10 | 95.600 | 156.169 | 37.563 | 1.00 | 43.89 | B |
| ATOM | 1029 | O | SER | B | 10 | 95.504 | 155.295 | 36.683 | 1.00 | 42.91 | B |
| ATOM | 1030 | N | LYS | B | 11 | 96.169 | 155.952 | 38.762 | 1.00 | 44.12 | B |
| ATOM | 1031 | CA | LYS | B | 11 | 96.781 | 154.668 | 39.118 | 1.00 | 43.94 | B |
| ATOM | 1032 | CB | LYS | B | 11 | 97.261 | 154.682 | 40.535 | 1.00 | 44.05 | B |
| ATOM | 1033 | CG | LYS | B | 11 | 98.282 | 153.619 | 40.748 | 1.00 | 46.02 | B |
| ATOM | 1034 | CD | LYS | B | 11 | 99.059 | 153.709 | 42.054 | 1.00 | 46.73 | B |
| ATOM | 1035 | CE | LYS | B | 11 | 100.254 | 52.805 | 41.887 | 1.00 | 47.43 | B |
| ATOM | 1036 | NZ | LYS | B | 11 | 100.602 | 152.178 | 43.150 | 1.00 | 52.18 | B |
| ATOM | 1037 | C | LYS | B | 11 | 95.745 | 153.577 | 38.982 | 1.00 | 45.09 | B |
| ATOM | 1038 | O | LYS | B | 11 | 95.954 | 152.522 | 38.333 | 1.00 | 43.17 | B |
| ATOM | 1039 | N | PHE | B | 12 | 94.599 | 153.859 | 39.592 | 1.00 | 45.86 | B |
| ATOM | 1040 | CA | PHE | B | 12 | 93.488 | 152.939 | 39.553 | 1.00 | 47.38 | B |
| ATOM | 1041 | CB | PHE | B | 12 | 92.313 | 153.465 | 40.343 | 1.00 | 47.92 | B |
| ATOM | 1042 | CG | PHE | B | 12 | 91.198 | 152.455 | 40.482 | 1.00 | 50.12 | B |
| ATOM | 1043 | CD1 | PHE | B | 12 | 91.306 | 151.413 | 41.407 | 1.00 | 49.35 | B |
| ATOM | 1044 | CD2 | PHE | B | 12 | 90.043 | 152.539 | 39.696 | 1.00 | 49.50 | B |
| ATOM | 1045 | CE1 | PHE | B | 12 | 90.271 | 150.480 | 41.546 | 1.00 | 49.93 | B |
| ATOM | 1046 | CE2 | PHE | B | 12 | 89.012 | 151.613 | 39.835 | 1.00 | 48.32 | B |
| ATOM | 1047 | CZ | PHE | B | 12 | 89.117 | 150.582 | 40.759 | 1.00 | 49.05 | B |
| ATOM | 1048 | C | PHE | B | 12 | 92.947 | 152.641 | 38.171 | 1.00 | 48.23 | B |
| ATOM | 1049 | O | PHE | B | 12 | 92.469 | 151.552 | 37.912 | 1.00 | 48.94 | B |
| ATOM | 1050 | N | GLU | B | 13 | 92.947 | 153.612 | 37.289 | 1.00 | 49.44 | B |
| ATOM | 1051 | CA | GLU | B | 13 | 92.373 | 153.314 | 36.009 | 1.00 | 51.19 | B |
| ATOM | 1052 | CB | GLU | B | 13 | 91.709 | 154.573 | 35.451 | 1.00 | 53.23 | B |
| ATOM | 1053 | CG | GLU | B | 13 | 90.535 | 155.081 | 36.340 | 1.00 | 55.04 | B |
| ATOM | 1054 | CD | GLU | B | 13 | 89.698 | 156.211 | 35.700 | 1.00 | 55.42 | B |
| ATOM | 1055 | OE1 | GLU | B | 13 | 90.061 | 156.573 | 34.554 | 1.00 | 54.92 | B |
| ATOM | 1056 | OE2 | GLU | B | 13 | 88.704 | 156.710 | 36.350 | 1.00 | 55.29 | B |
| ATOM | 1057 | C | GLU | B | 13 | 93.390 | 152.747 | 35.056 | 1.00 | 51.78 | B |
| ATOM | 1058 | O | GLU | B | 13 | 93.027 | 152.037 | 34.122 | 1.00 | 52.30 | B |
| ATOM | 1059 | N | ASN | B | 14 | 94.664 | 153.020 | 35.325 | 1.00 | 52.10 | B |
| ATOM | 1060 | CA | ASN | B | 14 | 95.753 | 152.577 | 34.453 | 1.00 | 52.76 | B |
| ATOM | 1061 | CB | ASN | B | 14 | 96.798 | 153.720 | 34.281 | 1.00 | 53.25 | B |
| ATOM | 1062 | CG | ASN | B | 14 | 96.326 | 154.829 | 33.305 | 1.00 | 54.53 | B |
| ATOM | 1063 | OD1 | ASN | B | 14 | 96.438 | 154.664 | 32.090 | 1.00 | 56.18 | B |
| ATOM | 1064 | ND2 | ASN | B | 14 | 95.789 | 155.935 | 33.831 | 1.00 | 52.16 | B |
| ATOM | 1065 | C | ASN | B | 14 | 96.502 | 151.274 | 34.758 | 1.00 | 53.12 | B |
| ATOM | 1066 | O | ASN | B | 14 | 96.710 | 150.530 | 33.835 | 1.00 | 53.84 | B |
| ATOM | 1067 | N | GLU | B | 15 | 96.905 | 150.999 | 36.011 | 1.00 | 53.07 | B |
| ATOM | 1068 | CA | GLU | B | 15 | 97.671 | 149.797 | 36.335 | 1.00 | 52.98 | B |
| ATOM | 1069 | CB | GLU | B | 15 | 98.254 | 149.861 | 37.738 | 1.00 | 55.54 | B |
| ATOM | 1070 | CG | GLU | B | 15 | 99.129 | 151.045 | 37.995 | 1.00 | 58.64 | B |
| ATOM | 1071 | CD | GLU | B | 15 | 100.031 | 150.833 | 39.208 | 1.00 | 59.81 | B |
| ATOM | 1072 | OE1 | GLU | B | 15 | 99.572 | 150.324 | 40.260 | 1.00 | 60.16 | B |
| ATOM | 1073 | OE2 | GLU | B | 15 | 101.218 | 151.193 | 39.089 | 1.00 | 62.17 | B |
| ATOM | 1074 | C | GLU | B | 15 | 96.879 | 148.515 | 36.240 | 1.00 | 52.47 | B |
| ATOM | 1075 | O | GLU | B | 15 | 95.756 | 148.420 | 36.735 | 1.00 | 52.55 | B |
| ATOM | 1076 | N | GLU | B | 16 | 97.503 | 147.524 | 35.635 | 1.00 | 51.07 | B |
| ATOM | 1077 | CA | GLU | B | 16 | 96.894 | 146.253 | 35.464 | 1.00 | 51.02 | B |
| ATOM | 1078 | CB | GLU | B | 16 | 97.916 | 145.327 | 34.825 | 1.00 | 53.86 | B |
| ATOM | 1079 | CG | GLU | B | 16 | 97.393 | 144.020 | 34.210 | 1.00 | 59.93 | B |
| ATOM | 1080 | CD | GLU | B | 16 | 98.557 | 143.189 | 33.593 | 1.00 | 63.63 | B |
| ATOM | 1081 | OE1 | GLU | B | 16 | 99.626 | 143.046 | 34.264 | 1.00 | 64.97 | B |
| ATOM | 1082 | OE2 | GLU | B | 16 | 98.416 | 142.684 | 32.437 | 1.00 | 65.34 | B |
| ATOM | 1083 | C | GLU | B | 16 | 96.425 | 145.721 | 36.818 | 1.00 | 50.16 | B |
| ATOM | 1084 | O | GLU | B | 16 | 95.341 | 145.112 | 36.920 | 1.00 | 49.54 | B |
| ATOM | 1085 | N | PHE | B | 17 | 97.209 | 145.966 | 37.872 | 1.00 | 48.87 | B |
| ATOM | 1086 | CA | PHE | B | 17 | 96.842 | 145.453 | 39.215 | 1.00 | 46.74 | B |
| ATOM | 1087 | CB | PHE | B | 17 | 97.724 | 146.025 | 40.326 | 1.00 | 43.02 | B |
| ATOM | 1088 | CG | PHE | B | 17 | 97.298 | 145.611 | 41.692 | 1.00 | 40.28 | B |
| ATOM | 1089 | CD1 | PHE | B | 17 | 97.483 | 144.287 | 42.129 | 1.00 | 39.18 | B |
| ATOM | 1090 | CD2 | PHE | B | 17 | 96.625 | 146.519 | 42.538 | 1.00 | 38.42 | B |
| ATOM | 1091 | CE1 | PHE | B | 17 | 96.991 | 143.874 | 43.405 | 1.00 | 38.73 | B |
| ATOM | 1092 | CE2 | PHE | B | 17 | 96.132 | 146.127 | 43.780 | 1.00 | 35.92 | B |
| ATOM | 1093 | CZ | PHE | B | 17 | 96.309 | 144.804 | 44.225 | 1.00 | 36.66 | B |
| ATOM | 1094 | C | PHE | B | 17 | 95.409 | 145.793 | 39.539 | 1.00 | 46.61 | B |
| ATOM | 1095 | O | PHE | B | 17 | 94.621 | 144.926 | 39.960 | 1.00 | 45.82 | B |
| ATOM | 1096 | N | PHE | B | 18 | 95.068 | 147.065 | 39.352 | 1.00 | 47.09 | B |
| ATOM | 1097 | CA | PHE | B | 18 | 93.697 | 147.465 | 39.637 | 1.00 | 48.31 | B |
| ATOM | 1098 | CB | PHE | B | 18 | 93.620 | 148.977 | 39.843 | 1.00 | 47.44 | B |
| ATOM | 1099 | CG | PHE | B | 18 | 94.337 | 149.454 | 41.087 | 1.00 | 46.14 | B |
| ATOM | 1100 | CD1 | PHE | B | 18 | 95.600 | 150.056 | 40.993 | 1.00 | 45.33 | B |
| ATOM | 1101 | CD2 | PHE | B | 18 | 93.769 | 149.265 | 42.350 | 1.00 | 44.62 | B |
| ATOM | 1102 | CE1 | PHE | B | 18 | 96.284 | 150.457 | 42.129 | 1.00 | 44.18 | B |
| ATOM | 1103 | CE2 | PHE | B | 18 | 94.432 | 149.658 | 43.491 | 1.00 | 43.94 | B |
| ATOM | 1104 | CZ | PHE | B | 18 | 95.702 | 150.260 | 43.385 | 1.00 | 44.07 | B |
| ATOM | 1105 | C | PHE | B | 18 | 92.695 | 246.988 | 38.570 | 1.00 | 48.93 | B |
| ATOM | 1106 | O | PHE | B | 18 | 91.705 | 146.325 | 38.893 | 1.00 | 49.06 | B |
| ATOM | 1107 | N | ARG | B | 19 | 92.962 | 147.307 | 37.314 | 1.00 | 49.92 | B |
| ATOM | 1108 | CA | ARG | B | 19 | 92.098 | 146.873 | 36.228 | 1.00 | 51.44 | B |
| ATOM | 1109 | CB | ARG | B | 19 | 92.825 | 147.058 | 34.881 | 1.00 | 53.15 | B |
| ATOM | 1110 | CG | ARG | B | 19 | 93.508 | 148.443 | 34.748 | 1.00 | 57.30 | B |
| ATOM | 1111 | CD | ARG | B | 19 | 94.090 | 148.752 | 33.367 | 1.00 | 58.35 | B |
| ATOM | 1112 | NE | ARG | B | 19 | 93.019 | 149.237 | 32.517 | 1.00 | 61.31 | B |
| ATOM | 1113 | CZ | ARG | B | 29 | 93.156 | 149.579 | 31.239 | 1.00 | 62.70 | B |
| ATOM | 1114 | NH1 | ARG | B | 19 | 94.356 | 149.494 | 30.657 | 1.00 | 63.13 | B |
| ATOM | 1115 | NH2 | ARG | B | 19 | 92.081 | 149.979 | 30.535 | 1.00 | 62.01 | B |
| ATOM | 1116 | C | ARG | B | 19 | 91.639 | 145.412 | 36.409 | 1.00 | 51.04 | B |
| ATOM | 1117 | O | ARG | B | 19 | 90.461 | 145.130 | 36.268 | 1.00 | 50.71 | B |
| ATOM | 1118 | N | LYS | B | 20 | 92.551 | 144.498 | 36.738 | 1.00 | 51.46 | B |
| ATOM | 1119 | CA | LYS | B | 20 | 92.200 | 143.080 | 36.930 | 1.00 | 52.10 | B |
| ATOM | 1120 | CB | LYS | B | 20 | 93.455 | 142.225 | 37.201 | 1.00 | 53.92 | B |
| ATOM | 1121 | CG | LYS | B | 20 | 94.215 | 141.839 | 35.935 | 1.00 | 57.20 | B |
| ATOM | 1122 | CD | LYS | B | 20 | 95.162 | 140.664 | 36.147 | 1.00 | 59.77 | B |
| ATOM | 1123 | CE | LYS | B | 20 | 95.932 | 140.407 | 34.852 | 1.00 | 62.33 | B |
| ATOM | 1124 | NZ | LYS | B | 20 | 97.397 | 140.083 | 35.034 | 1.00 | 64.96 | B |
| ATOM | 1125 | C | LYS | B | 20 | 91.252 | 142.870 | 38.081 | 1.00 | 51.91 | B |
| ATOM | 1126 | O | LYS | B | 20 | 90.684 | 141.794 | 38.242 | 1.00 | 51.79 | B |
| ATOM | 1127 | N | LEU | B | 21 | 91.091 | 143.884 | 38.910 | 1.00 | 51.11 | B |
| ATOM | 1128 | CA | LEU | B | 21 | 90.223 | 143.702 | 40.033 | 1.00 | 51.24 | B |
| ATOM | 1129 | CB | LEU | B | 21 | 91.011 | 143.928 | 41.328 | 1.00 | 50.68 | B |
| ATOM | 1130 | CG | LEU | B | 21 | 91.796 | 142.820 | 42.047 | 1.00 | 49.87 | B |
| ATOM | 1131 | CD1 | LEU | B | 21 | 91.758 | 141.582 | 41.231 | 1.00 | 48.95 | B |
| ATOM | 1132 | CD2 | LEU | B | 21 | 93.255 | 143.261 | 42.313 | 1.00 | 48.70 | B |
| ATOM | 1133 | C | LEU | B | 21 | 89.030 | 144.621 | 39.980 | 1.00 | 51.53 | B |
| ATOM | 1134 | O | LEU | B | 21 | 88.214 | 144.612 | 40.884 | 1.00 | 51.01 | B |
| ATOM | 1135 | N | SER | B | 22 | 88.903 | 145.395 | 38.916 | 1.00 | 52.46 | B |
| ATOM | 1136 | CA | SER | B | 22 | 87.813 | 146.349 | 38.866 | 1.00 | 55.27 | B |
| ATOM | 1137 | CB | SER | B | 22 | 88.245 | 147.593 | 38.123 | 1.00 | 54.99 | B |
| ATOM | 1138 | OG | SER | B | 22 | 88.882 | 147.201 | 36.931 | 1.00 | 57.43 | B |
| ATOM | 1139 | C | SER | B | 22 | 86.490 | 145.857 | 38.350 | 1.00 | 56.67 | B |
| ATOM | 1140 | O | SER | B | 22 | 85.494 | 146.537 | 36.529 | 1.00 | 56.06 | B |
| ATOM | 1141 | N | ARG | B | 23 | 86.483 | 144.676 | 37.735 | 1.00 | 60.02 | B |
| ATOM | 1142 | CA | ARG | B | 23 | 85.240 | 144.034 | 37.261 | 1.00 | 63.10 | B |
| ATOM | 1143 | CB | ARG | B | 23 | 85.379 | 143.497 | 35.837 | 1.00 | 66.91 | B |
| ATOM | 1144 | CG | ARG | B | 23 | 85.420 | 144.606 | 34.784 | 1.00 | 72.92 | B |
| ATOM | 1145 | CD | ARG | B | 23 | 86.713 | 145.492 | 34.797 | 1.00 | 77.51 | B |
| ATOM | 1146 | NE | ARG | B | 23 | 86.499 | 146.760 | 34.071 | 1.00 | 81.54 | B |
| ATOM | 1147 | CZ | ARG | B | 23 | 87.413 | 147.366 | 33.303 | 1.00 | 84.01 | B |
| ATOM | 1148 | NH1 | ARG | B | 23 | 88.621 | 146.822 | 33.151 | 1.00 | 85.11 | B |
| ATOM | 1149 | NH2 | ARG | B | 23 | 87.121 | 148.511 | 32.674 | 1.00 | 84.22 | B |
| ATOM | 1150 | C | ARG | B | 23 | 84.971 | 142.883 | 38.204 | 1.00 | 62.72 | B |
| ATOM | 1151 | O | ARG | B | 23 | 85.900 | 142.314 | 38.767 | 1.00 | 62.78 | B |
| ATOM | 1152 | N | GLU | B | 24 | 83.717 | 142.524 | 38.395 | 1.00 | 62.75 | B |
| ATOM | 1153 | CA | CLU | B | 24 | 83.453 | 141.431 | 39.322 | 1.00 | 62.61 | B |
| ATOM | 1154 | CB | CLU | B | 24 | 81.976 | 141.164 | 39.413 | 1.00 | 64.73 | B |
| ATOM | 1155 | CG | GLU | B | 24 | 81.274 | 142.236 | 40.144 | 1.00 | 69.19 | B |
| ATOM | 1156 | CD | GLU | B | 24 | 79.785 | 142.225 | 39.842 | 1.00 | 71.77 | B |
| ATOM | 1157 | OE1 | GLU | B | 24 | 79.207 | 141.096 | 39.906 | 1.00 | 71.62 | B |
| ATOM | 1158 | OE2 | GLU | B | 24 | 79.225 | 143.341 | 39.546 | 1.00 | 72.88 | B |
| ATOM | 1159 | C | GLU | B | 24 | 84.150 | 140.130 | 38.979 | 1.00 | 61.07 | B |
| ATOM | 1160 | O | GLU | B | 24 | 84.259 | 139.757 | 37.803 | 1.00 | 60.58 | B |
| ATOM | 1161 | N | CYS | B | 25 | 84.639 | 139.442 | 40.012 | 1.00 | 59.00 | B |
| ATOM | 1162 | CA | CYS | B | 25 | 85.294 | 138.150 | 39.817 | 1.00 | 56.20 | B |
| ATOM | 1163 | CB | CYS | B | 25 | 86.751 | 138.284 | 39.339 | 1.00 | 56.47 | B |
| ATOM | 1164 | SG | CYS | B | 25 | 87.737 | 139.559 | 40.149 | 1.00 | 54.54 | B |
| ATOM | 1165 | C | CYS | B | 25 | 85.238 | 137.253 | 41.011 | 1.00 | 53.95 | B |
| ATOM | 1166 | O | CYS | B | 25 | 84.807 | 137.634 | 42.101 | 1.00 | 52.79 | B |
| ATOM | 1167 | N | GLU | B | 26 | 85.660 | 136.031 | 40.765 | 1.00 | 52.99 | B |
| ATOM | 1168 | CA | GLU | B | 26 | 85.626 | 134.999 | 41.775 | 1.00 | 53.79 | B |
| ATOM | 1169 | CB | GLU | B | 26 | 86.110 | 133.674 | 41.164 | 1.00 | 54.09 | B |
| ATOM | 1170 | CG | GLU | B | 26 | 85.943 | 132.478 | 42.073 | 1.00 | 55.84 | B |
| ATOM | 1171 | CD | GLU | B | 26 | 86.369 | 131.191 | 41.396 | 1.00 | 57.81 | B |
| ATOM | 1172 | OE1 | GLU | B | 26 | 86.448 | 130.159 | 42.098 | 1.00 | 58.26 | B |
| ATOM | 1173 | OE2 | GLU | B | 26 | 86.618 | 131.198 | 40.168 | 1.00 | 58.78 | B |
| ATOM | 1174 | C | GLU | B | 26 | 86.432 | 135.347 | 43.028 | 1.00 | 52.92 | B |
| ATOM | 1175 | O | GLU | B | 26 | 87.614 | 135.617 | 42.947 | 1.00 | 54.16 | B |
| ATOM | 1176 | N | ILE | B | 27 | 85.772 | 135.386 | 44.167 | 1.00 | 51.25 | B |
| ATOM | 1177 | CA | ILE | B | 27 | 86.426 | 135.665 | 45.409 | 1.00 | 51.10 | B |
| ATOM | 1178 | CB | ILE | B | 27 | 85.905 | 136.924 | 46.064 | 1.00 | 50.50 | B |
| ATOM | 1179 | CG2 | ILE | B | 27 | 86.394 | 138.117 | 45.255 | 1.00 | 48.81 | B |
| ATOM | 1180 | CG1 | ILE | B | 27 | 84.376 | 136.852 | 46.186 | 1.00 | 51.16 | B |
| ATOM | 1181 | CD1 | ILE | B | 27 | 83.775 | 137.730 | 47.239 | 1.00 | 51.25 | B |
| ATOM | 1182 | C | ILE | B | 27 | 86.146 | 134.470 | 46.275 | 1.00 | 51.70 | B |
| ATOM | 1183 | O | ILE | B | 27 | 85.421 | 133.580 | 45.882 | 1.00 | 52.87 | B |
| ATOM | 1184 | N | LYS | B | 28 | 86.723 | 134.427 | 47.457 | 1.00 | 52.12 | B |
| ATOM | 1185 | CA | LYS | B | 28 | 86.526 | 133.265 | 48.295 | 1.00 | 51.78 | B |
| ATOM | 1186 | CB | LYS | B | 28 | 87.220 | 132.091 | 47.625 | 1.00 | 52.22 | B |
| ATOM | 1187 | CG | LYS | B | 28 | 87.139 | 130.799 | 48.386 | 1.00 | 54.39 | B |
| ATOM | 1188 | CD | LYS | B | 28 | 87.799 | 129.632 | 47.611 | 1.00 | 54.80 | B |
| ATOM | 1189 | CE | LYS | B | 28 | 87.075 | 129.308 | 46.318 | 1.00 | 55.46 | B |
| ATOM | 1190 | NZ | LYS | B | 28 | 87.247 | 127.843 | 46.039 | 1.00 | 56.08 | B |
| ATOM | 1191 | C | LYS | B | 28 | 87.071 | 133.510 | 49.692 | 1.00 | 51.45 | B |
| ATOM | 1192 | O | LYS | B | 28 | 88.106 | 134.159 | 49.864 | 1.00 | 51.87 | B |
| ATOM | 1193 | N | TYR | B | 29 | 86.343 | 133.032 | 50.690 | 1.00 | 50.85 | B |
| ATOM | 1194 | CA | TYR | B | 29 | 86.742 | 133.184 | 52.093 | 1.00 | 50.45 | B |
| ATOM | 1195 | CB | TYR | B | 29 | 85.610 | 132.729 | 53.025 | 1.00 | 48.79 | B |
| ATOM | 1196 | CG | TYR | B | 29 | 85.978 | 132.719 | 54.483 | 1.00 | 47.91 | B |
| ATOM | 1197 | CD1 | TYR | B | 29 | 86.271 | 133.903 | 55.149 | 1.00 | 47.78 | B |
| ATOM | 1198 | CE1 | TYR | B | 29 | 86.567 | 133.916 | 56.479 | 1.00 | 46.61 | B |
| ATOM | 1199 | CD2 | TYR | B | 29 | 86.008 | 131.529 | 55.214 | 1.00 | 48.00 | B |
| ATOM | 1200 | CE2 | TYR | B | 29 | 86.319 | 131.534 | 56.568 | 1.00 | 47.25 | B |
| ATOM | 1201 | CZ | TYR | B | 29 | 86.587 | 132.734 | 57.191 | 1.00 | 47.12 | B |
| ATOM | 1202 | OH | TYR | B | 29 | 86.838 | 132.774 | 58.544 | 1.00 | 47.97 | B |
| ATOM | 1203 | C | TYR | B | 29 | 87.972 | 132.315 | 52.313 | 1.00 | 49.70 | B |
| ATOM | 1204 | O | TYR | B | 29 | 87.977 | 131.155 | 51.935 | 1.00 | 50.39 | B |
| ATOM | 1205 | N | THR | B | 30 | 88.996 | 132.877 | 52.931 | 1.00 | 49.00 | B |
| ATOM | 1206 | CA | THR | B | 30 | 90.253 | 132.158 | 53.153 | 1.00 | 48.82 | B |
| ATOM | 1207 | CB | THR | B | 30 | 91.465 | 132.984 | 52.637 | 1.00 | 46.51 | B |
| ATOM | 1208 | OG1 | THR | B | 30 | 91.570 | 134.176 | 53.409 | 1.00 | 42.97 | B |
| ATOM | 1209 | CG2 | THR | B | 30 | 91.321 | 133.341 | 51.227 | 1.00 | 42.82 | B |
| ATOM | 1210 | C | THR | B | 30 | 90.536 | 131.835 | 54.623 | 1.00 | 49.92 | B |
| ATOM | 1211 | O | THR | B | 30 | 91.538 | 131.175 | 54.930 | 1.00 | 49.12 | B |
| ATOM | 1212 | N | GLY | B | 31 | 89.653 | 132.304 | 55.514 | 1.00 | 51.53 | B |
| ATOM | 1213 | CA | GLY | B | 31 | 89.816 | 132.084 | 56.939 | 1.00 | 52.52 | B |
| ATOM | 1214 | C | GLY | B | 31 | 89.682 | 130.642 | 57.355 | 1.00 | 54.78 | B |
| ATOM | 1215 | O | GLY | B | 31 | 88.982 | 129.869 | 56.701 | 1.00 | 54.89 | B |
| ATOM | 1216 | N | PHE | B | 32 | 90.354 | 130.289 | 58.451 | 1.00 | 56.56 | B |
| ATOM | 1217 | CA | PHE | B | 32 | 90.351 | 128.944 | 59.022 | 1.00 | 58.77 | B |
| ATOM | 1218 | CB | PHE | B | 32 | 89.197 | 128.787 | 60.000 | 1.00 | 58.26 | B |
| ATOM | 1219 | CG | PHE | B | 32 | 89.142 | 129.861 | 61.036 | 1.00 | 58.23 | B |
| ATOM | 1220 | CD1 | PHE | B | 32 | 88.646 | 131.123 | 60.722 | 1.00 | 57.75 | B |
| ATOM | 1221 | CD2 | PHE | B | 32 | 89.610 | 129.621 | 62.324 | 1.00 | 58.16 | B |
| ATOM | 1222 | CE1 | PHE | B | 32 | 88.615 | 132.136 | 61.668 | 1.00 | 57.66 | B |
| ATOM | 1223 | CE2 | PHE | B | 32 | 89.585 | 130.627 | 63.286 | 1.00 | 57.32 | B |
| ATOM | 1224 | CZ | PHE | B | 32 | 89.085 | 131.890 | 62.956 | 1.00 | 57.72 | B |
| ATOM | 1225 | C | PHE | B | 32 | 90.308 | 127.816 | 57.998 | 1.00 | 60.90 | B |
| ATOM | 1226 | O | PHE | B | 32 | 89.518 | 126.897 | 58.114 | 1.00 | 61.45 | B |
| ATOM | 1227 | N | ARG | B | 33 | 91.193 | 127.877 | 57.014 | 1.00 | 63.46 | B |
| ATOM | 1228 | CA | ARG | B | 33 | 91.280 | 126.885 | 55.952 | 1.00 | 65.63 | B |
| ATOM | 1229 | CB | ARG | B | 33 | 92.428 | 127.290 | 55.025 | 1.00 | 65.57 | B |
| ATOM | 1230 | CG | ARG | B | 33 | 92.274 | 126.881 | 53.558 | 1.00 | 65.30 | B |
| ATOM | 1231 | CD | ARG | B | 33 | 92.006 | 128.101 | 52.690 | 1.00 | 65.03 | B |
| ATOM | 1232 | NE | ARG | B | 33 | 92.794 | 128.119 | 51.459 | 1.00 | 65.60 | B |
| ATOM | 1233 | CZ | ARG | B | 33 | 92.941 | 127.074 | 50.643 | 1.00 | 65.82 | B |
| ATOM | 1234 | NH1 | ARG | B | 33 | 92.356 | 125.924 | 50.946 | 1.00 | 66.23 | B |
| ATOM | 1235 | NH2 | ARG | B | 33 | 93.644 | 127.178 | 49.516 | 1.00 | 65.16 | B |
| ATOM | 1236 | C | ARG | B | 33 | 91.474 | 125.420 | 56.411 | 1.00 | 67.50 | B |
| ATOM | 1237 | O | ARG | B | 33 | 91.164 | 124.484 | 55.665 | 1.00 | 67.53 | B |
| ATOM | 1238 | N | ASP | B | 34 | 91.973 | 125.206 | 57.623 | 1.00 | 69.28 | B |
| ATOM | 1239 | CA | ASP | B | 34 | 92.212 | 123.839 | 58.079 | 1.00 | 71.26 | B |
| ATOM | 1240 | CB | ASP | B | 34 | 93.459 | 123.802 | 58.982 | 1.00 | 72.24 | B |
| ATOM | 1241 | CG | ASP | B | 34 | 93.415 | 124.843 | 60.120 | 1.00 | 73.41 | B |
| ATOM | 1242 | OD1 | ASP | B | 34 | 92.634 | 125.818 | 60.044 | 1.00 | 73.10 | B |
| ATOM | 1243 | OD2 | ASP | B | 34 | 94.191 | 124.702 | 61.096 | 1.00 | 74.15 | B |
| ATOM | 1244 | C | ASP | B | 34 | 91.046 | 123.140 | 58.773 | 1.00 | 72.25 | B |
| ATOM | 1245 | O | ASP | B | 34 | 91.014 | 121.915 | 58.836 | 1.00 | 72.40 | B |
| ATOM | 1246 | N | ARG | B | 35 | 90.087 | 123.918 | 59.273 | 1.00 | 73.27 | B |
| ATOM | 1247 | CA | ARG | B | 35 | 88.921 | 123.396 | 59.981 | 1.00 | 73.09 | B |
| ATOM | 1248 | CB | ARG | B | 35 | 88.293 | 124.509 | 60.825 | 1.00 | 74.05 | B |
| ATOM | 1249 | CG | ARG | B | 35 | 89.015 | 124.782 | 62.118 | 1.00 | 75.48 | B |
| ATOM | 1250 | CD | ARG | B | 35 | 90.516 | 124.834 | 61.880 | 1.00 | 77.31 | B |
| ATOM | 1251 | NE | ARG | B | 35 | 91.312 | 125.064 | 63.087 | 1.00 | 78.61 | B |
| ATOM | 1252 | CZ | ARG | B | 35 | 90.975 | 125.891 | 64.082 | 1.00 | 79.44 | B |
| ATOM | 1253 | NH1 | ARG | B | 35 | 89.824 | 126.586 | 64.047 | 1.00 | 78.53 | B |
| ATOM | 1254 | NH2 | ARG | B | 35 | 91.816 | 126.042 | 65.110 | 1.00 | 79.17 | B |
| ATOM | 1255 | C | ARG | B | 35 | 87.867 | 122.827 | 59.054 | 1.00 | 73.23 | B |
| ATOM | 1256 | O | ARG | B | 35 | 87.846 | 123.112 | 57.859 | 1.00 | 72.96 | B |
| ATOM | 1257 | N | PRO | B | 36 | 86.966 | 122.004 | 59.601 | 1.00 | 73.77 | B |
| ATOM | 1258 | CD | PRO | B | 36 | 86.892 | 121.556 | 61.004 | 1.00 | 73.93 | B |
| ATOM | 1259 | CA | PRO | B | 36 | 85.894 | 121.402 | 58.787 | 1.00 | 73.37 | B |
| ATOM | 1260 | CB | PRO | B | 36 | 85.114 | 120.557 | 59.794 | 1.00 | 73.68 | B |
| ATOM | 1261 | CG | PRO | B | 36 | 86.137 | 120.259 | 60.865 | 1.00 | 73.75 | B |
| ATOM | 1262 | C | PRO | B | 36 | 85.026 | 122.510 | 58.178 | 1.00 | 72.99 | B |
| ATOM | 1263 | O | PRO | B | 36 | 84.856 | 123.585 | 58.761 | 1.00 | 72.83 | B |
| ATOM | 1264 | N | HIS | B | 37 | 84.463 | 122.245 | 57.016 | 1.00 | 72.42 | B |
| ATOM | 1265 | CA | HIS | B | 37 | 83.658 | 123.246 | 56.370 | 1.00 | 72.25 | B |
| ATOM | 1266 | CB | HIS | B | 37 | 83.035 | 122.679 | 55.106 | 1.00 | 72.57 | B |
| ATOM | 1267 | CG | HIS | B | 37 | 82.234 | 123.677 | 54.329 | 1.00 | 73.40 | B |
| ATOM | 1268 | CD2 | HIS | B | 37 | 80.898 | 123.788 | 54.125 | 1.00 | 73.84 | B |
| ATOM | 1269 | ND1 | HIS | B | 37 | 82.818 | 124.695 | 53.607 | 1.00 | 73.65 | B |
| ATOM | 1270 | CE1 | HIS | B | 37 | 81.878 | 125.384 | 52.982 | 1.00 | 74.35 | B |
| ATOM | 1271 | NE2 | HIS | B | 37 | 80.704 | 124.854 | 53.279 | 1.00 | 74.42 | B |
| ATOM | 1272 | C | HIS | B | 37 | 82.566 | 123.875 | 57.230 | 1.00 | 72.38 | B |
| ATOM | 1273 | O | HIS | B | 37 | 82.408 | 125.086 | 57.216 | 1.00 | 72.32 | B |
| ATOM | 1274 | N | GLU | B | 38 | 81.808 | 123.073 | 57.970 | 1.00 | 72.59 | B |
| ATOM | 1275 | CA | GLU | B | 38 | 80.718 | 123.612 | 58.791 | 1.00 | 72.79 | B |
| ATOM | 1276 | CB | GLU | B | 38 | 80.045 | 122.499 | 59.596 | 1.00 | 75.04 | B |
| ATOM | 1277 | CG | GLU | B | 38 | 79.298 | 121.474 | 58.725 | 1.00 | 79.74 | B |
| ATOM | 1278 | CD | GLU | B | 38 | 80.201 | 120.799 | 57.653 | 1.00 | 81.96 | B |
| ATOM | 1279 | OE1 | GLU | B | 38 | 81.283 | 120.238 | 58.024 | 1.00 | 82.49 | B |
| ATOM | 1280 | OE2 | GLU | B | 38 | 79.811 | 120.834 | 56.444 | 1.00 | 82.34 | B |
| ATOM | 1281 | C | GLU | B | 38 | 81.200 | 124.704 | 59.736 | 1.00 | 71.51 | B |
| ATOM | 1282 | O | GLU | B | 38 | 80.522 | 125.717 | 59.927 | 1.00 | 70.50 | B |
| ATOM | 1283 | N | GLU | B | 39 | 82.371 | 124.489 | 60.325 | 1.00 | 69.93 | B |
| ATOM | 1284 | CA | GLU | B | 39 | 82.945 | 125.459 | 61.238 | 1.00 | 68.96 | B |
| ATOM | 1285 | CB | GLU | B | 39 | 84.214 | 124.873 | 61.877 | 1.00 | 69.44 | B |
| ATOM | 1286 | CG | GLU | B | 39 | 84.710 | 125.665 | 63.096 | 1.00 | 70.10 | B |
| ATOM | 1287 | CD | GLU | B | 39 | 86.017 | 125.135 | 63.659 | 1.00 | 70.60 | B |
| ATOM | 1288 | OE1 | GLU | B | 39 | 86.138 | 123.897 | 63.776 | 1.00 | 70.28 | B |
| ATOM | 1289 | OE2 | GLU | B | 39 | 86.917 | 125.952 | 63.993 | 1.00 | 70.98 | B |
| ATOM | 1290 | C | GLU | B | 39 | 83.288 | 126.750 | 60.468 | 1.00 | 67.70 | B |
| ATOM | 1291 | O | GLU | B | 39 | 82.949 | 127.876 | 60.876 | 1.00 | 65.76 | B |
| ATOM | 1292 | N | ARG | B | 40 | 83.961 | 126.562 | 59.338 | 1.00 | 66.73 | B |
| ATOM | 1293 | CA | ARG | B | 40 | 84.364 | 127.684 | 58.505 | 1.00 | 66.17 | B |
| ATOM | 1294 | CB | ARG | B | 40 | 85.014 | 127.180 | 57.206 | 1.00 | 65.76 | B |
| ATOM | 1295 | CG | ARG | B | 40 | 86.286 | 126.352 | 57.425 | 1.00 | 65.18 | B |
| ATOM | 1296 | CD | ARG | B | 40 | 87.123 | 126.286 | 56.160 | 1.00 | 65.24 | B |
| ATOM | 1297 | NE | ARG | B | 40 | 87.584 | 127.618 | 55.772 | 1.00 | 65.48 | B |
| ATOM | 1298 | CZ | ARG | B | 40 | 87.433 | 128.140 | 54.563 | 1.00 | 65.09 | B |
| ATOM | 1299 | NH1 | ARG | B | 40 | 86.834 | 127.449 | 53.610 | 1.00 | 65.05 | B |
| ATOM | 1300 | NH2 | ARG | B | 40 | 87.874 | 129.355 | 54.306 | 1.00 | 65.28 | B |
| ATOM | 1301 | C | ARG | B | 40 | 83.142 | 128.534 | 58.216 | 1.00 | 65.92 | B |
| ATOM | 1302 | O | ARG | B | 40 | 83.201 | 129.748 | 58.189 | 1.00 | 65.81 | B |
| ATOM | 1303 | N | GLN | B | 41 | 82.015 | 127.884 | 58.005 | 1.00 | 66.05 | B |
| ATOM | 1304 | CA | GLN | B | 41 | 80.794 | 128.611 | 57.773 | 1.00 | 65.24 | B |
| ATOM | 1305 | CB | GLN | B | 41 | 79.665 | 127.633 | 57.519 | 1.00 | 65.01 | B |
| ATOM | 1306 | CG | GLN | B | 41 | 79.474 | 127.283 | 56.081 | 1.00 | 64.85 | B |
| ATOM | 1307 | CD | GLN | B | 41 | 78.664 | 126.031 | 55.938 | 1.00 | 65.03 | B |
| ATOM | 1308 | OE1 | GLN | B | 41 | 78.015 | 125.815 | 54.924 | 1.00 | 65.58 | B |
| ATOM | 1309 | NE2 | GLN | B | 41 | 78.705 | 125.181 | 56.961 | 1.00 | 65.67 | B |
| ATOM | 1310 | C | GLN | B | 41 | 80.468 | 129.476 | 58.995 | 1.00 | 64.99 | B |
| ATOM | 1311 | O | GLN | B | 41 | 80.133 | 130.652 | 58.858 | 1.00 | 64.94 | B |
| ATOM | 1312 | N | ALA | B | 42 | 80.582 | 128.900 | 60.185 | 1.00 | 64.78 | B |
| ATOM | 1313 | CA | ALA | B | 42 | 80.256 | 129.627 | 61.394 | 1.00 | 65.73 | B |
| ATOM | 1314 | CB | ALA | B | 42 | 80.311 | 128.720 | 62.607 | 1.00 | 65.42 | B |
| ATOM | 1315 | C | ALA | B | 42 | 81.232 | 130.746 | 61.562 | 1.00 | 66.87 | B |
| ATOM | 1316 | O | ALA | B | 42 | 80.828 | 131.923 | 61.648 | 1.00 | 67.37 | B |
| ATOM | 1317 | N | ARG | B | 43 | 82.519 | 130.375 | 61.613 | 1.00 | 67.18 | B |
| ATOM | 1318 | CA | ARG | B | 43 | 83.628 | 131.329 | 61.772 | 1.00 | 66.69 | B |
| ATOM | 1319 | CB | ARG | B | 43 | 84.967 | 130.683 | 61.418 | 1.00 | 67.58 | B |
| ATOM | 1320 | CG | ARG | B | 43 | 85.419 | 129.523 | 62.281 | 1.00 | 67.89 | B |
| ATOM | 1321 | CD | ARG | B | 43 | 85.777 | 129.989 | 63.632 | 1.00 | 68.29 | B |
| ATOM | 1322 | NE | ARG | B | 43 | 86.375 | 128.923 | 64.431 | 1.00 | 70.19 | B |
| ATOM | 1323 | CZ | ARG | B | 43 | 86.491 | 128.976 | 65.761 | 1.00 | 70.83 | B |
| ATOM | 1324 | NH1 | ARG | B | 43 | 86.040 | 130.042 | 66.433 | 1.00 | 70.87 | B |
| ATOM | 1325 | NH2 | ARG | B | 43 | 87.062 | 127.976 | 66.426 | 1.00 | 71.26 | B |
| ATOM | 1326 | C | ARG | B | 43 | 83.424 | 132.512 | 60.843 | 1.00 | 66.39 | B |
| ATOM | 1327 | O | ARG | B | 43 | 83.487 | 133.659 | 61.271 | 1.00 | 66.61 | B |
| ATOM | 1328 | N | PHE | B | 44 | 83.185 | 132.225 | 59.565 | 1.00 | 65.61 | B |
| ATOM | 1329 | CA | PHE | B | 44 | 82.940 | 133.270 | 58.583 | 1.00 | 65.32 | B |
| ATOM | 1330 | CB | PHE | B | 44 | 82.441 | 132.695 | 57.264 | 1.00 | 62.26 | B |
| ATOM | 1331 | CG | PHE | B | 44 | 82.147 | 133.743 | 56.257 | 1.00 | 59.78 | B |
| ATOM | 1332 | CD1 | PHE | B | 44 | 83.147 | 134.580 | 55.811 | 1.00 | 57.96 | B |
| ATOM | 1333 | CD2 | PHE | B | 44 | 80.865 | 133.953 | 55.808 | 1.00 | 59.46 | B |
| ATOM | 1334 | CE1 | PHE | B | 44 | 82.876 | 135.611 | 54.942 | 1.00 | 57.67 | B |
| ATOM | 1335 | CE2 | PHE | B | 44 | 80.589 | 134.999 | 54.924 | 1.00 | 58.76 | B |
| ATOM | 1336 | CZ | PHE | B | 44 | 81.600 | 135.828 | 54.495 | 1.00 | 57.85 | B |
| ATOM | 1337 | C | PHE | B | 44 | 81.902 | 134.271 | 59.075 | 1.00 | 66.64 | B |
| ATOM | 1338 | O | PHE | B | 44 | 82.213 | 135.443 | 59.282 | 1.00 | 67.88 | B |
| ATOM | 1339 | N | GLN | B | 45 | 80.668 | 133.811 | 59.251 | 1.00 | 67.89 | B |
| ATOM | 1340 | CA | GLN | B | 45 | 79.579 | 134.675 | 59.725 | 1.00 | 69.10 | B |
| ATOM | 1341 | CB | GLN | B | 45 | 78.284 | 133.876 | 59.845 | 1.00 | 70.23 | B |
| ATOM | 1342 | CG | GLN | B | 45 | 77.449 | 133.795 | 58.582 | 1.00 | 70.50 | B |
| ATOM | 1343 | CD | GLN | B | 45 | 75.996 | 133.560 | 58.928 | 1.00 | 71.65 | B |
| ATOM | 1344 | OE1 | GLN | B | 45 | 75.372 | 134.353 | 59.658 | 1.00 | 71.13 | B |
| ATOM | 1345 | NE2 | GLN | B | 45 | 75.447 | 132.463 | 58.427 | 1.00 | 71.55 | B |
| ATOM | 1346 | C | GLN | B | 45 | 79.823 | 135.400 | 61.058 | 1.00 | 69.10 | B |
| ATOM | 1347 | O | GLN | B | 45 | 79.489 | 136.581 | 61.197 | 1.00 | 67.58 | B |
| ATOM | 1348 | N | ASN | B | 46 | 80.380 | 134.691 | 62.037 | 1.00 | 69.74 | B |
| ATOM | 1349 | CA | ASN | B | 46 | 80.658 | 135.312 | 63.331 | 1.00 | 71.25 | B |
| ATOM | 1350 | CB | ASN | B | 46 | 81.464 | 134.363 | 64.222 | 1.00 | 73.59 | B |
| ATOM | 1351 | CG | ASN | B | 46 | 80.831 | 132.975 | 64.316 | 1.00 | 76.88 | B |
| ATOM | 1352 | OD1 | ASN | B | 46 | 79.593 | 132.836 | 64.502 | 1.00 | 77.54 | B |
| ATOM | 1353 | ND2 | ASN | B | 46 | 81.676 | 131.929 | 64.198 | 1.00 | 77.90 | B |
| ATOM | 1354 | C | ASN | B | 46 | 81.448 | 136.590 | 63.105 | 1.00 | 70.73 | B |
| ATOM | 1355 | O | ASN | B | 46 | 81.014 | 137.684 | 63.469 | 1.00 | 70.24 | B |
| ATOM | 1356 | N | ALA | B | 47 | 82.610 | 136.416 | 62.482 | 1.00 | 70.70 | B |
| ATOM | 1357 | CA | ALA | B | 47 | 83.529 | 137.493 | 62.139 | 1.00 | 70.02 | B |
| ATOM | 1358 | CB | ALA | B | 47 | 84.748 | 136.912 | 61.449 | 1.00 | 69.09 | B |
| ATOM | 1359 | C | ALA | B | 47 | 82.871 | 138.553 | 61.245 | 1.00 | 69.89 | B |
| ATOM | 1360 | O | ALA | B | 47 | 83.159 | 139.744 | 61.362 | 1.00 | 69.01 | B |
| ATOM | 1361 | N | CYS | B | 48 | 82.005 | 138.135 | 60.335 | 1.00 | 69.97 | B |
| ATOM | 1362 | CA | CYS | B | 48 | 81.349 | 139.128 | 59.507 | 1.00 | 71.29 | B |
| ATOM | 1363 | CB | CYS | B | 48 | 80.444 | 138.464 | 58.500 | 1.00 | 70.17 | B |
| ATOM | 1364 | SG | CYS | B | 48 | 81.294 | 138.152 | 56.980 | 1.00 | 71.02 | B |
| ATOM | 1365 | C | CYS | B | 48 | 80.542 | 140.065 | 60.405 | 1.00 | 72.54 | B |
| ATOM | 1366 | O | CYS | B | 48 | 80.606 | 141.288 | 60.284 | 1.00 | 72.77 | B |
| ATOM | 1367 | N | ARG | B | 49 | 79.790 | 139.484 | 61.328 | 1.00 | 74.03 | B |
| ATOM | 1368 | CA | ARG | B | 49 | 78.998 | 140.282 | 62.233 | 1.00 | 74.97 | B |
| ATOM | 1369 | CB | ARG | B | 49 | 78.101 | 139.372 | 63.097 | 1.00 | 77.06 | B |
| ATOM | 1370 | CG | ARG | B | 49 | 76.943 | 138.769 | 62.252 | 1.00 | 80.55 | B |
| ATOM | 1371 | CD | ARG | B | 49 | 75.948 | 137.846 | 63.005 | 1.00 | 83.50 | B |
| ATOM | 1372 | NE | ARG | B | 49 | 76.391 | 136.448 | 63.122 | 1.00 | 85.65 | B |
| ATOM | 1373 | CZ | ARG | B | 49 | 76.993 | 135.934 | 64.200 | 1.00 | 86.52 | B |
| ATOM | 1374 | NH1 | ARG | B | 49 | 77.233 | 136.702 | 65.269 | 1.00 | 86.57 | B |
| ATOM | 1375 | NH2 | ARG | B | 49 | 77.343 | 134.648 | 64.220 | 1.00 | 85.89 | B |
| ATOM | 1376 | C | ARG | B | 49 | 79.955 | 141.132 | 63.052 | 1.00 | 74.46 | B |
| ATOM | 1377 | O | ARG | B | 49 | 79.567 | 142.150 | 63.614 | 1.00 | 75.44 | B |
| ATOM | 1378 | N | ASP | B | 50 | 81.216 | 140.728 | 63.089 | 1.00 | 73.32 | B |
| ATOM | 1379 | CA | ASP | B | 50 | 82.251 | 141.473 | 63.807 | 1.00 | 72.24 | B |
| ATOM | 1380 | CB | ASP | B | 50 | 83.353 | 140.523 | 64.255 | 1.00 | 75.07 | B |
| ATOM | 1381 | CG | ASP | B | 50 | 83.293 | 140.212 | 65.714 | 1.00 | 77.06 | B |
| ATOM | 1382 | OD1 | ASP | B | 50 | 83.372 | 141.176 | 66.517 | 1.00 | 79.00 | B |
| ATOM | 1383 | OD2 | ASP | B | 50 | 83.177 | 139.009 | 66.052 | 1.00 | 78.31 | B |
| ATOM | 1384 | C | ASP | B | 50 | 82.880 | 142.533 | 62.892 | 1.00 | 70.36 | B |
| ATOM | 1385 | O | ASP | B | 50 | 83.895 | 143.156 | 63.243 | 1.00 | 69.60 | B |
| ATOM | 1386 | N | GLY | B | 51 | 82.295 | 142.680 | 61.702 | 1.00 | 68.34 | B |
| ATOM | 1387 | CA | GLY | B | 51 | 82.772 | 143.644 | 60.724 | 1.00 | 65.81 | B |
| ATOM | 1388 | C | GLY | B | 51 | 84.126 | 143.349 | 60.116 | 1.00 | 63.79 | B |
| ATOM | 1389 | O | GLY | B | 51 | 84.863 | 144.260 | 59.761 | 1.00 | 64.02 | B |
| ATOM | 1390 | N | ARG | B | 52 | 84.461 | 142.078 | 59.975 | 1.00 | 61.77 | B |
| ATOM | 1391 | CA | ARG | B | 52 | 85.751 | 141.743 | 59.412 | 1.00 | 59.31 | B |
| ATOM | 1392 | CB | ARG | B | 52 | 86.807 | 141.805 | 60.510 | 1.00 | 58.80 | B |
| ATOM | 1393 | CG | ARG | B | 52 | 86.774 | 140.675 | 61.495 | 1.00 | 59.84 | B |
| ATOM | 1394 | CD | ARG | B | 52 | 87.679 | 140.956 | 62.736 | 1.00 | 61.39 | B |
| ATOM | 1395 | NE | ARG | B | 52 | 87.594 | 139.874 | 63.727 | 1.00 | 61.79 | B |
| ATOM | 1396 | CZ | ARG | B | 52 | 87.965 | 138.619 | 63.470 | 1.00 | 63.21 | B |
| ATOM | 1397 | NH1 | ARG | B | 52 | 88.452 | 138.319 | 62.265 | 1.00 | 63.53 | B |
| ATOM | 1398 | NH2 | ARG | B | 52 | 87.824 | 137.644 | 64.386 | 1.00 | 64.57 | B |
| ATOM | 1399 | C | ARG | B | 52 | 85.755 | 140.384 | 58.744 | 1.00 | 58.15 | B |
| ATOM | 1400 | O | ARG | B | 52 | 84.891 | 139.577 | 58.998 | 1.00 | 58.37 | B |
| ATOM | 1401 | N | SER | B | 53 | 86.708 | 140.149 | 57.851 | 1.00 | 57.38 | B |
| ATOM | 1402 | CA | SER | B | 53 | 86.832 | 138.873 | 57.182 | 1.00 | 56.09 | B |
| ATOM | 1403 | CB | SER | B | 53 | 85.583 | 138.564 | 56.387 | 1.00 | 56.89 | B |
| ATOM | 1404 | OG | SER | B | 53 | 85.766 | 137.336 | 55.726 | 1.00 | 56.69 | B |
| ATOM | 1405 | C | SER | B | 53 | 88.014 | 138.892 | 56.252 | 1.00 | 55.08 | B |
| ATOM | 1406 | O | SER | B | 53 | 88.552 | 139.933 | 55.972 | 1.00 | 54.86 | B |
| ATOM | 1407 | N | GLU | B | 54 | 88.396 | 137.727 | 55.751 | 1.00 | 55.01 | B |
| ATOM | 1408 | CA | GLU | B | 54 | 89.523 | 137.596 | 54.850 | 1.00 | 54.44 | B |
| ATOM | 1409 | CB | GLU | B | 54 | 90.560 | 136.695 | 55.464 | 1.00 | 56.77 | B |
| ATOM | 1410 | CG | GLU | B | 54 | 91.058 | 137.123 | 56.790 | 1.00 | 58.77 | B |
| ATOM | 1411 | CD | GLU | B | 54 | 91.801 | 135.994 | 57.448 | 1.00 | 62.59 | B |
| ATOM | 1412 | OE1 | GLU | B | 54 | 92.231 | 135.035 | 56.719 | 1.00 | 64.59 | B |
| ATOM | 1413 | OE2 | GLU | B | 54 | 91.960 | 136.059 | 58.691 | 1.00 | 63.68 | B |
| ATOM | 1414 | C | GLU | B | 54 | 89.075 | 136.952 | 53.582 | 1.00 | 53.36 | B |
| ATOM | 1415 | O | GLU | B | 54 | 88.502 | 135.885 | 53.614 | 1.00 | 53.35 | B |
| ATOM | 1416 | N | ILE | B | 55 | 89.371 | 137.568 | 52.454 | 1.00 | 53.28 | B |
| ATOM | 1417 | CA | ILE | B | 55 | 88.957 | 137.009 | 51.175 | 1.00 | 53.30 | B |
| ATOM | 1418 | CB | ILE | B | 55 | 87.693 | 137.688 | 50.687 | 1.00 | 53.70 | B |
| ATOM | 1419 | CG2 | ILE | B | 55 | 87.324 | 137.178 | 49.308 | 1.00 | 55.38 | B |
| ATOM | 1420 | CG1 | ILE | B | 55 | 86.539 | 137.321 | 51.625 | 1.00 | 55.46 | B |
| ATOM | 1421 | CD1 | ILE | B | 55 | 85.369 | 138.266 | 51.532 | 1.00 | 57.62 | B |
| ATOM | 1422 | C | ILE | B | 55 | 90.024 | 137.139 | 50.130 | 1.00 | 52.56 | B |
| ATOM | 1423 | O | ILE | B | 55 | 90.822 | 138.067 | 50.178 | 1.00 | 53.73 | B |
| ATOM | 1424 | N | ALA | B | 56 | 90.067 | 136.196 | 49.192 | 1.00 | 50.85 | B |
| ATOM | 1425 | CA | ALA | B | 56 | 91.075 | 136.267 | 48.132 | 1.00 | 49.87 | B |
| ATOM | 1426 | CB | ALA | B | 56 | 91.952 | 135.004 | 48.157 | 1.00 | 47.31 | B |
| ATOM | 1427 | C | ALA | B | 56 | 90.437 | 136.420 | 46.759 | 1.00 | 48.94 | B |
| ATOM | 1428 | O | ALA | B | 56 | 89.374 | 135.866 | 46.516 | 1.00 | 49.06 | B |
| ATOM | 1429 | N | PHE | B | 57 | 91.043 | 137.197 | 45.872 | 1.00 | 48.08 | B |
| ATOM | 1430 | CA | PHE | B | 57 | 90.505 | 137.254 | 44.516 | 1.00 | 46.75 | B |
| ATOM | 1431 | CB | PHE | B | 57 | 90.947 | 138.528 | 43.779 | 1.00 | 46.52 | B |
| ATOM | 1432 | CG | PHE | B | 57 | 90.175 | 139.755 | 44.166 | 1.00 | 45.81 | B |
| ATOM | 1433 | CD1 | PHE | B | 57 | 90.686 | 140.644 | 45.071 | 1.00 | 44.87 | B |
| ATOM | 1434 | CD2 | PHE | B | 57 | 88.924 | 139.990 | 43.642 | 1.00 | 47.56 | B |
| ATOM | 1435 | CE1 | PHE | B | 57 | 89.994 | 141.737 | 45.455 | 1.00 | 45.39 | B |
| ATOM | 1436 | CE2 | PHE | B | 57 | 88.190 | 141.112 | 44.025 | 1.00 | 48.36 | B |
| ATOM | 1437 | CZ | PHE | B | 57 | 88.735 | 141.988 | 44.939 | 1.00 | 47.48 | B |
| ATOM | 1438 | C | PHE | B | 57 | 91.169 | 136.002 | 43.919 | 1.00 | 45.73 | B |
| ATOM | 1439 | O | PHE | B | 57 | 92.362 | 136.016 | 43.607 | 1.00 | 46.43 | B |
| ATOM | 1440 | N | VAL | B | 58 | 90.395 | 134.924 | 43.796 | 1.00 | 43.81 | B |
| ATOM | 1441 | CA | VAL | B | 58 | 90.862 | 133.617 | 43.315 | 1.00 | 42.31 | B |
| ATOM | 1442 | CB | VAL | B | 58 | 89.691 | 132.640 | 43.094 | 1.00 | 40.92 | B |
| ATOM | 1443 | CG1 | VAL | B | 58 | 90.223 | 131.325 | 42.661 | 1.00 | 39.47 | B |
| ATOM | 1444 | CG2 | VAL | B | 58 | 88.887 | 132.483 | 44.351 | 1.00 | 39.88 | B |
| ATOM | 1445 | C | VAL | B | 58 | 91.727 | 133.532 | 42.080 | 1.00 | 41.94 | B |
| ATOM | 1446 | O | VAL | B | 58 | 92.782 | 132.914 | 42.100 | 1.00 | 42.36 | B |
| ATOM | 1447 | N | ALA | B | 59 | 91.281 | 134.132 | 40.995 | 1.00 | 41.77 | B |
| ATOM | 1448 | CA | ALA | B | 59 | 92.062 | 134.037 | 39.804 | 1.00 | 41.12 | B |
| ATOM | 1449 | CB | ALA | B | 59 | 91.362 | 134.770 | 38.684 | 1.00 | 40.04 | B |
| ATOM | 1450 | C | ALA | B | 59 | 93.501 | 134.546 | 39.992 | 1.00 | 41.73 | B |
| ATOM | 1451 | O | ALA | B | 59 | 94.432 | 133.864 | 39.573 | 1.00 | 43.44 | B |
| ATOM | 1452 | N | THR | B | 60 | 93.708 | 135.699 | 40.628 | 1.00 | 40.47 | B |
| ATOM | 1453 | CA | THR | B | 60 | 95.063 | 136.217 | 40.737 | 1.00 | 39.15 | B |
| ATOM | 1454 | CB | THR | B | 60 | 95.106 | 137.774 | 40.755 | 1.00 | 39.73 | B |
| ATOM | 1455 | OG1 | THR | B | 60 | 94.416 | 138.232 | 41.919 | 1.00 | 41.85 | B |
| ATOM | 1456 | CG2 | THR | B | 60 | 94.447 | 138.394 | 39.507 | 1.00 | 39.68 | B |
| ATOM | 1457 | C | THR | B | 60 | 95.709 | 135.775 | 42.013 | 1.00 | 39.63 | B |
| ATOM | 1458 | O | THR | B | 60 | 96.947 | 135.839 | 42.178 | 1.00 | 39.60 | B |
| ATOM | 1459 | N | GLY | B | 61 | 94.890 | 135.338 | 42.955 | 1.00 | 39.04 | B |
| ATOM | 1460 | CA | GLY | B | 61 | 95.473 | 134.981 | 44.228 | 1.00 | 38.37 | B |
| ATOM | 1461 | C | GLY | B | 61 | 95.740 | 136.227 | 45.083 | 1.00 | 38.15 | B |
| ATOM | 1462 | O | GLY | B | 61 | 96.516 | 136.151 | 45.999 | 1.00 | 39.31 | B |
| ATOM | 1463 | N | THR | B | 62 | 95.113 | 137.372 | 44.814 | 1.00 | 37.86 | B |
| ATOM | 1464 | CA | THR | B | 62 | 95.368 | 138.548 | 45.648 | 1.00 | 37.63 | B |
| ATOM | 1465 | CB | THR | B | 62 | 95.044 | 139.867 | 44.944 | 1.00 | 36.89 | B |
| ATOM | 1466 | OG1 | THR | B | 62 | 95.862 | 140.016 | 43.785 | 1.00 | 34.63 | B |
| ATOM | 1467 | CG2 | THR | B | 62 | 95.270 | 141.034 | 45.929 | 1.00 | 33.75 | B |
| ATOM | 1468 | C | THR | B | 62 | 94.498 | 138.490 | 46.897 | 1.00 | 38.25 | B |
| ATOM | 1469 | O | THR | B | 62 | 93.252 | 138.604 | 46.789 | 1.00 | 39.19 | B |
| ATOM | 1470 | N | ASN | B | 63 | 95.143 | 138.335 | 48.059 | 1.00 | 37.15 | B |
| ATOM | 1471 | CA | ASN | B | 63 | 94.456 | 138.250 | 49.333 | 1.00 | 37.43 | B |
| ATOM | 1472 | CB | ASN | B | 63 | 95.245 | 137.371 | 50.312 | 1.00 | 39.66 | B |
| ATOM | 1473 | CG | ASN | B | 63 | 95.216 | 135.850 | 49.958 | 1.00 | 40.27 | B |
| ATOM | 1474 | OD1 | ASN | B | 63 | 94.801 | 134.998 | 50.775 | 1.00 | 39.85 | B |
| ATOM | 1475 | ND2 | ASN | B | 63 | 95.686 | 135.512 | 48.759 | 1.00 | 41.39 | B |
| ATOM | 1476 | C | ASN | B | 63 | 94.268 | 139.610 | 49.979 | 1.00 | 38.04 | B |
| ATOM | 1477 | O | ASN | B | 63 | 95.090 | 140.497 | 49.804 | 1.00 | 37.34 | B |
| ATOM | 1478 | N | LEU | B | 64 | 93.195 | 139.751 | 50.760 | 1.00 | 38.90 | B |
| ATOM | 1479 | CA | LEU | B | 64 | 92.867 | 140.997 | 51.479 | 1.00 | 39.07 | B |
| ATOM | 1480 | CB | LEU | B | 64 | 91.951 | 141.868 | 50.627 | 1.00 | 37.54 | B |
| ATOM | 1481 | CG | LEU | B | 64 | 92.428 | 142.550 | 49.356 | 1.00 | 36.91 | B |
| ATOM | 1482 | CD1 | LEU | B | 64 | 91.227 | 143.286 | 48.726 | 1.00 | 34.87 | B |
| ATOM | 1483 | CD2 | LEU | B | 64 | 93.612 | 143.520 | 49.669 | 1.00 | 34.27 | B |
| ATOM | 1484 | C | LEU | B | 64 | 92.130 | 140.727 | 52.809 | 1.00 | 39.99 | B |
| ATOM | 1485 | O | LEU | B | 64 | 91.303 | 139.824 | 52.880 | 1.00 | 39.78 | B |
| ATOM | 1486 | N | SER | B | 65 | 92.454 | 141.460 | 53.868 | 1.00 | 41.20 | B |
| ATOM | 1487 | CA | SER | B | 65 | 91.716 | 141.321 | 55.127 | 1.00 | 42.55 | B |
| ATOM | 1488 | CB | SER | B | 65 | 92.552 | 141.539 | 56.374 | 1.00 | 43.37 | B |
| ATOM | 1489 | OG | SER | B | 65 | 93.387 | 140.435 | 56.644 | 1.00 | 47.76 | B |
| ATOM | 1490 | C | SER | B | 65 | 90.854 | 142.539 | 54.953 | 1.00 | 44.25 | B |
| ATOM | 1491 | O | SER | B | 65 | 91.343 | 143.599 | 54.467 | 1.00 | 44.43 | B |
| ATOM | 1492 | N | LEU | B | 66 | 89.580 | 142.400 | 55.323 | 1.00 | 45.74 | B |
| ATOM | 1493 | CA | LEU | B | 66 | 88.603 | 143.470 | 55.181 | 1.00 | 46.14 | B |
| ATOM | 1494 | CB | LEU | B | 66 | 87.478 | 142.969 | 54.303 | 1.00 | 44.70 | B |
| ATOM | 1495 | CG | LEU | B | 66 | 88.013 | 142.533 | 52.965 | 1.00 | 45.50 | B |
| ATOM | 1496 | CD1 | LEU | B | 66 | 86.890 | 141.905 | 52.144 | 1.00 | 46.47 | B |
| ATOM | 1497 | CD2 | LEU | B | 66 | 88.592 | 143.763 | 52.259 | 1.00 | 44.33 | B |
| ATOM | 1498 | C | LEU | B | 66 | 88.036 | 143.953 | 56.502 | 1.00 | 47.76 | B |
| ATOM | 1499 | O | LEU | B | 66 | 87.934 | 143.182 | 57.458 | 1.00 | 47.28 | B |
| ATOM | 1500 | N | GLN | B | 67 | 87.692 | 145.242 | 56.545 | 1.00 | 50.35 | B |
| ATOM | 1501 | CA | GLN | B | 67 | 87.064 | 145.882 | 57.716 | 1.00 | 52.69 | B |
| ATOM | 1502 | CB | GLN | B | 67 | 87.922 | 146.982 | 58.285 | 1.00 | 53.61 | B |
| ATOM | 1503 | CG | GLN | B | 67 | 89.255 | 146.505 | 58.688 | 1.00 | 55.18 | B |
| ATOM | 1504 | CD | GLN | B | 67 | 89.428 | 146.576 | 60.172 | 1.00 | 56.56 | B |
| ATOM | 1505 | OE1 | GLN | B | 67 | 89.432 | 145.525 | 60.858 | 1.00 | 55.32 | B |
| ATOM | 1506 | NE2 | GLN | B | 67 | 89.574 | 147.828 | 60.701 | 1.00 | 55.85 | B |
| ATOM | 1507 | C | GLN | B | 67 | 85.839 | 146.542 | 57.157 | 1.00 | 53.47 | B |
| ATOM | 1508 | O | GLN | B | 67 | 85.969 | 147.453 | 56.336 | 1.00 | 53.55 | B |
| ATOM | 1509 | N | PHE | B | 68 | 84.666 | 146.111 | 57.608 | 1.00 | 54.77 | B |
| ATOM | 1510 | CA | PHE | B | 68 | 83.436 | 146.662 | 57.089 | 1.00 | 56.82 | B |
| ATOM | 1511 | CB | PHE | B | 68 | 82.409 | 145.565 | 56.946 | 1.00 | 55.19 | B |
| ATOM | 1512 | CG | PHE | B | 68 | 82.961 | 144.283 | 56.415 | 1.00 | 54.28 | B |
| ATOM | 1513 | CD1 | PHE | B | 68 | 82.955 | 143.150 | 57.201 | 1.00 | 53.73 | B |
| ATOM | 1514 | CD2 | PHE | B | 68 | 83.418 | 144.185 | 55.110 | 1.00 | 54.49 | B |
| ATOM | 1515 | CE1 | PHE | B | 68 | 83.388 | 141.937 | 56.698 | 1.00 | 53.96 | B |
| ATOM | 1516 | CE2 | PHE | B | 68 | 83.856 | 142.958 | 54.600 | 1.00 | 53.64 | B |
| ATOM | 1517 | CZ | PHE | B | 68 | 83.837 | 141.840 | 55.401 | 1.00 | 53.92 | B |
| ATOM | 1518 | C | PHE | B | 68 | 82.837 | 147.790 | 57.911 | 1.00 | 59.22 | B |
| ATOM | 1519 | O | PHE | B | 68 | 81.798 | 147.631 | 58.526 | 1.00 | 60.20 | B |
| ATOM | 1520 | N | PHE | B | 69 | 83.495 | 148.939 | 57.909 | 1.00 | 61.89 | B |
| ATOM | 1521 | CA | PHE | B | 69 | 83.030 | 150.118 | 58.629 | 1.00 | 63.64 | B |
| ATOM | 1522 | CB | PHE | B | 69 | 83.750 | 150.288 | 59.952 | 1.00 | 65.44 | B |
| ATOM | 1523 | CG | PHE | B | 69 | 83.811 | 149.049 | 60.756 | 1.00 | 67.63 | B |
| ATOM | 1524 | CD1 | PHE | B | 69 | 84.854 | 148.152 | 60.584 | 1.00 | 68.53 | B |
| ATOM | 1525 | CD2 | PHE | B | 69 | 82.814 | 148.775 | 61.689 | 1.00 | 69.61 | B |
| ATOM | 1526 | CE1 | PHE | B | 69 | 84.919 | 146.983 | 61.337 | 1.00 | 71.40 | B |
| ATOM | 1527 | CE2 | PHE | B | 69 | 82.849 | 147.607 | 62.464 | 1.00 | 71.43 | B |
| ATOM | 1528 | CZ | PHE | B | 69 | 83.911 | 146.699 | 62.291 | 1.00 | 71.82 | B |
| ATOM | 1529 | C | PHE | B | 69 | 3.446 | 151.239 | 57.733 | 1.00 | 64.01 | B |
| ATOM | 1530 | O | PHE | B | 69 | 84.230 | 151.045 | 56.819 | 1.00 | 63.52 | |
| ATOM | 1531 | N | PRO | B | 70 | 82.914 | 152.432 | 57.965 | 1.00 | 65.16 | |
| ATOM | 1532 | CD | PRO | B | 70 | 81.741 | 152.743 | 58.810 | 1.00 | 64.94 | |
| ATOM | 1533 | CA | PRO | B | 70 | 83.298 | 153.567 | 57.109 | 1.00 | 64.95 | B |
| ATOM | 1534 | CB | PRO | B | 70 | 82.138 | 154.526 | 57.297 | 1.00 | 64.77 | B |
| ATOM | 1535 | CG | PRO | B | 70 | 81.700 | 154.242 | 58.760 | 1.00 | 64.74 | B |
| ATOM | 1536 | C | PRO | B | 70 | 84.652 | 154.184 | 57.511 | 1.00 | 64.81 | B |
| ATOM | 1537 | O | PRO | B | 70 | 85.057 | 154.105 | 58.671 | 1.00 | 64.69 | B |
| ATOM | 1538 | N | THR | B | 80 | 80.191 | 147.471 | 66.168 | 1.00 | 84.83 | B |
| ATOM | 1539 | CA | THR | B | 80 | 79.318 | 146.620 | 65.350 | 1.00 | 84.93 | B |
| ATOM | 1540 | CB | THR | B | 80 | 77.950 | 146.376 | 66.040 | 1.00 | 84.65 | B |
| ATOM | 1541 | OG1 | THR | B | 80 | 78.153 | 145.593 | 67.218 | 1.00 | 84.11 | B |
| ATOM | 1542 | CG2 | THR | B | 80 | 76.978 | 145.642 | 65.107 | 1.00 | 83.48 | B |
| ATOM | 1543 | C | THR | B | 80 | 79.047 | 147.217 | 63.968 | 1.00 | 85.47 | B |
| ATOM | 1544 | O | THR | B | 80 | 78.818 | 148.432 | 63.827 | 1.00 | 85.75 | B |
| ATOM | 1545 | N | PRO | B | 81 | 79.048 | 146.365 | 62.924 | 1.00 | 85.45 | B |
| ATOM | 1546 | CD | PRO | B | 81 | 79.373 | 144.926 | 62.970 | 1.00 | 85.18 | B |
| ATOM | 1547 | CA | PRO | B | 81 | 78.807 | 146.812 | 61.540 | 1.00 | 84.94 | B |
| ATOM | 1548 | CB | PRO | B | 81 | 79.214 | 145.596 | 60.705 | 1.00 | 84.96 | B |
| ATOM | 1549 | CG | PRO | B | 81 | 78.887 | 144.438 | 61.620 | 1.00 | 84.82 | B |
| ATOM | 1550 | C | PRO | B | 81 | 77.382 | 147.258 | 61.257 | 1.00 | 84.34 | B |
| ATOM | 1551 | O | PRO | B | 81 | 76.478 | 146.973 | 62.032 | 1.00 | 83.98 | B |
| ATOM | 1552 | N | SER | B | 82 | 77.206 | 147.960 | 60.139 | 1.00 | 84.06 | B |
| ATOM | 1553 | CA | SER | B | 82 | 75.904 | 148.463 | 59.713 | 1.00 | 83.91 | B |
| ATOM | 1554 | CB | SER | B | 82 | 76.026 | 149.895 | 59.202 | 1.00 | 83.67 | B |
| ATOM | 1555 | OG | SER | B | 82 | 75.178 | 150.126 | 58.088 | 1.00 | 82.17 | B |
| ATOM | 1556 | C | SER | B | 82 | 75.409 | 147.603 | 58.583 | 1.00 | 84.47 | B |
| ATOM | 1557 | O | SER | B | 82 | 76.198 | 146.937 | 57.936 | 1.00 | 85.15 | B |
| ATOM | 1558 | N | ARG | B | 83 | 74.107 | 147.626 | 58.321 | 1.00 | 84.95 | B |
| ATOM | 1559 | CA | ARG | B | 83 | 73.554 | 146.829 | 57.227 | 1.00 | 84.77 | B |
| ATOM | 1560 | CB | ARG | B | 83 | 72.015 | 146.940 | 57.198 | 1.00 | 87.07 | B |
| ATOM | 1561 | CG | ARG | B | 83 | 71.303 | 145.828 | 56.393 | 1.00 | 90.02 | B |
| ATOM | 1562 | CD | ARG | B | 83 | 71.592 | 144.407 | 56.974 | 1.00 | 92.82 | B |
| ATOM | 1563 | NE | ARG | B | 83 | 70.680 | 143.997 | 58.060 | 1.00 | 96.02 | B |
| ATOM | 1564 | CZ | ARG | B | 83 | 70.613 | 144.526 | 59.299 | 1.00 | 96.88 | B |
| ATOM | 1565 | NH1 | ARG | B | 83 | 71.414 | 145.520 | 59.684 | 1.00 | 96.52 | B |
| ATOM | 1566 | NH2 | ARG | B | 83 | 69.714 | 144.068 | 60.171 | 1.00 | 96.91 | B |
| ATOM | 1567 | C | ARG | B | 83 | 74.138 | 147.327 | 55.901 | 1.00 | 83.44 | B |
| ATOM | 1568 | O | ARG | B | 83 | 74.139 | 146.614 | 54.896 | 1.00 | 83.00 | B |
| ATOM | 1569 | N | GLU | B | 84 | 74.636 | 148.562 | 55.917 | 1.00 | 82.14 | B |
| ATOM | 1570 | CA | GLU | B | 84 | 75.238 | 149.189 | 54.735 | 1.00 | 80.65 | B |
| ATOM | 1571 | CB | GLU | B | 84 | 75.527 | 150.673 | 54.996 | 1.00 | 79.97 | B |
| ATOM | 1572 | CG | GLU | B | 84 | 76.099 | 151.392 | 53.777 | 1.00 | 78.95 | B |
| ATOM | 1573 | CD | GLU | B | 84 | 76.520 | 152.822 | 54.073 | 1.00 | 78.24 | B |
| ATOM | 1574 | OE1 | GLU | B | 84 | 76.022 | 153.404 | 55.064 | 1.00 | 77.51 | B |
| ATOM | 1575 | OE2 | GLU | B | 84 | 77.341 | 153.364 | 53.302 | 1.00 | 77.09 | B |
| ATOM | 1576 | C | GLU | B | 84 | 76.548 | 148.499 | 54.368 | 1.00 | 79.87 | B |
| ATOM | 1577 | O | GLU | B | 84 | 76.877 | 148.356 | 53.188 | 1.00 | 79.47 | B |
| ATOM | 1578 | N | TYR | B | 85 | 77.285 | 148.087 | 55.399 | 1.00 | 79.02 | B |
| ATOM | 1579 | CA | TYR | B | 85 | 78.566 | 147.416 | 55.238 | 1.00 | 78.62 | B |
| ATOM | 1580 | CB | TYR | B | 85 | 79.562 | 147.993 | 56.257 | 1.00 | 78.36 | B |
| ATOM | 1581 | CG | TYR | B | 85 | 79.995 | 149.391 | 55.866 | 1.00 | 77.49 | B |
| ATOM | 1582 | CD1 | TYR | B | 85 | 79.234 | 150.509 | 56.209 | 1.00 | 76.77 | B |
| ATOM | 1583 | CE1 | TYR | B | 85 | 79.554 | 151.759 | 55.735 | 1.00 | 76.30 | B |
| ATOM | 1584 | CD2 | TYR | B | 85 | 81.095 | 149.575 | 55.042 | 1.00 | 76.99 | B |
| ATOM | 1585 | CE2 | TYR | B | 85 | 81.420 | 150.819 | 54.560 | 1.00 | 77.32 | B |
| ATOM | 1586 | CZ | TYR | B | 85 | 80.646 | 151.911 | 54.903 | 1.00 | 77.28 | B |
| ATOM | 1587 | OH | TYR | B | 85 | 80.969 | 153.136 | 54.369 | 1.00 | 77.05 | B |
| ATOM | 1588 | C | TYR | B | 85 | 78.496 | 145.877 | 55.311 | 1.00 | 78.28 | B |
| ATOM | 1589 | O | TYR | B | 85 | 79.206 | 145.178 | 54.585 | 1.00 | 77.78 | B |
| ATOM | 1590 | N | VAL | B | 86 | 77.636 | 145.371 | 56.186 | 1.00 | 77.71 | B |
| ATOM | 1591 | CA | VAL | B | 86 | 77.416 | 143.939 | 56.349 | 1.00 | 77.57 | B |
| ATOM | 1592 | CB | VAL | B | 86 | 77.821 | 143.440 | 57.787 | 1.00 | 76.90 | B |
| ATOM | 1593 | CG1 | VAL | B | 86 | 77.504 | 141.964 | 57.943 | 1.00 | 75.81 | B |
| ATOM | 1594 | CG2 | VAL | B | 86 | 79.298 | 143.687 | 58.036 | 1.00 | 75.69 | B |
| ATOM | 1595 | C | VAL | B | 86 | 75.907 | 143.775 | 56.143 | 1.00 | 77.97 | B |
| ATOM | 1596 | O | VAL | B | 86 | 75.123 | 143.884 | 57.095 | 1.00 | 78.29 | B |
| ATOM | 1597 | N | ASP | B | 87 | 75.506 | 143.534 | 54.899 | 1.00 | 77.95 | B |
| ATOM | 1598 | CA | ASP | B | 87 | 74.099 | 143.395 | 54.574 | 1.00 | 78.43 | B |
| ATOM | 1599 | CB | ASP | B | 87 | 73.778 | 144.082 | 53.242 | 1.00 | 78.62 | B |
| ATOM | 1600 | CG | ASP | B | 87 | 72.269 | 144.203 | 52.987 | 1.00 | 78.59 | B |
| ATOM | 1601 | OD1 | ASP | B | 87 | 71.483 | 143.408 | 53.555 | 1.00 | 78.34 | B |
| ATOM | 1602 | OD2 | ASP | B | 87 | 71.868 | 145.092 | 52.211 | 1.00 | 77.85 | B |
| ATOM | 1603 | C | ASP | B | 87 | 73.639 | 141.959 | 54.495 | 1.00 | 78.79 | B |
| ATOM | 1604 | O | ASP | B | 87 | 73.973 | 141.244 | 53.559 | 1.00 | 78.80 | B |
| ATOM | 1605 | N | LEU | B | 88 | 72.828 | 141.553 | 55.461 | 1.00 | 79.63 | B |
| ATOM | 1606 | CA | LEU | B | 88 | 72.317 | 140.192 | 55.481 | 1.00 | 79.84 | B |
| ATOM | 1607 | CB | LEU | B | 88 | 72.352 | 139.660 | 56.912 | 1.00 | 80.22 | B |
| ATOM | 1608 | CG | LEU | B | 88 | 73.697 | 139.000 | 57.196 | 1.00 | 80.45 | B |
| ATOM | 1609 | CD1 | LEU | B | 88 | 73.764 | 138.527 | 58.635 | 1.00 | 80.52 | B |
| ATOM | 1610 | CD2 | LEU | B | 88 | 73.873 | 137.825 | 56.214 | 1.00 | 80.48 | B |
| ATOM | 1611 | C | LEU | B | 88 | 70.932 | 140.001 | 54.875 | 1.00 | 79.34 | B |
| ATOM | 1612 | O | LEU | B | 88 | 70.607 | 138.915 | 54.430 | 1.00 | 78.94 | B |
| ATOM | 1613 | N | GLU | B | 89 | 70.133 | 141.059 | 54.838 | 1.00 | 79.99 | B |
| ATOM | 1614 | CA | GLU | B | 89 | 68.772 | 140.987 | 54.287 | 1.00 | 80.96 | B |
| ATOM | 1615 | CB | GLU | B | 89 | 67.978 | 142.221 | 54.700 | 1.00 | 82.71 | B |
| ATOM | 1616 | CG | GLU | B | 89 | 68.021 | 142.474 | 56.207 | 1.00 | 86.83 | B |
| ATOM | 1617 | CD | GLU | B | 89 | 67.568 | 143.900 | 56.581 | 1.00 | 89.95 | B |
| ATOM | 1618 | OE1 | GLU | B | 89 | 68.279 | 144.893 | 56.235 | 1.00 | 90.69 | B |
| ATOM | 1619 | OE2 | GLU | B | 89 | 66.485 | 144.026 | 57.218 | 1.00 | 92.24 | B |
| ATOM | 1620 | C | GLU | B | 89 | 68.777 | 140.864 | 52.773 | 1.00 | 79.90 | B |
| ATOM | 1621 | O | GLU | B | 89 | 68.589 | 139.785 | 52.244 | 1.00 | 79.51 | B |
| ATOM | 1622 | N | ARG | B | 90 | 68.974 | 141.986 | 52.091 | 1.00 | 79.20 | B |
| ATOM | 1623 | CA | ARG | B | 90 | 69.049 | 142.058 | 50.629 | 1.00 | 78.20 | B |
| ATOM | 1624 | CB | ARG | B | 90 | 70.397 | 142.655 | 50.242 | 1.00 | 77.85 | B |
| ATOM | 1625 | CG | ARG | B | 90 | 70.739 | 142.517 | 48.789 | 1.00 | 77.44 | B |
| ATOM | 1626 | CD | ARG | B | 90 | 71.816 | 143.492 | 48.447 | 1.00 | 77.54 | B |
| ATOM | 1627 | NE | ARG | B | 90 | 72.170 | 143.393 | 47.043 | 1.00 | 78.26 | B |
| ATOM | 1628 | CZ | ARG | B | 90 | 72.830 | 144.330 | 46.364 | 1.00 | 78.24 | B |
| ATOM | 1629 | NH1 | ARG | B | 90 | 73.206 | 145.447 | 46.972 | 1.00 | 78.82 | B |
| ATOM | 1630 | NH2 | ARG | B | 90 | 73.114 | 144.155 | 45.076 | 1.00 | 77.27 | B |
| ATOM | 1631 | C | ARG | B | 90 | 68.814 | 140.778 | 49.811 | 1.00 | 78.24 | B |
| ATOM | 1632 | O | ARG | B | 90 | 68.019 | 140.773 | 48.884 | 1.00 | 78.52 | B |
| ATOM | 1633 | N | GLU | B | 91 | 69.541 | 139.711 | 50.115 | 1.00 | 77.80 | B |
| ATOM | 1634 | CA | GLU | B | 91 | 69.376 | 138.442 | 49.417 | 1.00 | 76.99 | B |
| ATOM | 1635 | CB | GLU | B | 91 | 70.272 | 138.413 | 48.182 | 1.00 | 76.08 | B |
| ATOM | 1636 | CG | GLU | B | 91 | 70.206 | 137.109 | 47.404 | 1.00 | 76.39 | B |
| ATOM | 1637 | CD | GLU | B | 91 | 70.649 | 137.273 | 45.941 | 1.00 | 77.24 | B |
| ATOM | 1638 | OE1 | GLU | B | 91 | 70.824 | 136.232 | 45.246 | 1.00 | 76.76 | B |
| ATOM | 1639 | OE2 | GLU | B | 91 | 70.810 | 138.443 | 45.480 | 1.00 | 76.80 | B |
| ATOM | 1640 | C | GLU | B | 91 | 69.729 | 137.324 | 50.415 | 1.00 | 77.02 | B |
| ATOM | 1641 | O | GLU | B | 91 | 70.707 | 137.430 | 51.161 | 1.00 | 77.92 | B |
| ATOM | 1642 | N | ALA | B | 92 | 68.927 | 136.269 | 50.460 | 1.00 | 76.25 | B |
| ATOM | 1643 | CA | ALA | B | 92 | 69.206 | 135.229 | 51.419 | 1.00 | 75.79 | B |
| ATOM | 1644 | CB | ALA | B | 92 | 67.910 | 134.556 | 51.849 | 1.00 | 76.04 | B |
| ATOM | 1645 | C | ALA | B | 92 | 70.179 | 134.221 | 50.853 | 1.00 | 75.43 | B |
| ATOM | 1646 | O | ALA | B | 92 | 70.267 | 134.037 | 49.631 | 1.00 | 74.54 | B |
| ATOM | 1647 | N | GLY | B | 93 | 70.914 | 133.581 | 51.759 | 1.00 | 74.87 | B |
| ATOM | 1648 | CA | GLY | B | 93 | 71.885 | 132.572 | 51.357 | 1.00 | 74.50 | B |
| ATOM | 1649 | C | GLY | B | 93 | 73.276 | 133.146 | 51.155 | 1.00 | 73.51 | B |
| ATOM | 1650 | O | GLY | B | 93 | 74.293 | 132.413 | 51.117 | 1.00 | 72.68 | B |
| ATOM | 1651 | N | LYS | B | 94 | 73.301 | 134.476 | 51.055 | 1.00 | 71.96 | B |
| ATOM | 1652 | CA | LYS | B | 94 | 74.537 | 135.189 | 50.835 | 1.00 | 70.01 | B |
| ATOM | 1653 | CB | LYS | B | 94 | 74.781 | 135.288 | 49.330 | 1.00 | 70.00 | B |
| ATOM | 1654 | CG | LYS | B | 94 | 73.587 | 135.746 | 48.535 | 1.00 | 68.65 | B |
| ATOM | 1655 | CD | LYS | B | 94 | 73.783 | 135.425 | 47.059 | 1.00 | 68.45 | B |
| ATOM | 1656 | CE | LYS | B | 94 | 74.301 | 136.630 | 46.318 | 1.00 | 69.21 | B |
| ATOM | 1657 | NZ | LYS | B | 94 | 74.275 | 136.491 | 44.838 | 1.00 | 69.89 | B |
| ATOM | 1658 | C | LYS | B | 94 | 74.577 | 136.570 | 51.482 | 1.00 | 68.04 | B |
| ATOM | 1659 | O | LYS | B | 94 | 73.560 | 137.224 | 51.645 | 1.00 | 68.14 | B |
| ATOM | 1660 | N | VAL | B | 95 | 75.778 | 136.993 | 51.854 | 1.00 | 65.82 | B |
| ATOM | 1661 | CA | VAL | B | 95 | 75.987 | 138.294 | 52.466 | 1.00 | 62.85 | B |
| ATOM | 1662 | CB | VAL | B | 95 | 76.847 | 138.169 | 53.741 | 1.00 | 61.60 | B |
| ATOM | 1663 | CG1 | VAL | B | 95 | 78.243 | 137.813 | 53.372 | 1.00 | 61.63 | B |
| ATOM | 1664 | CG2 | VAL | B | 95 | 76.804 | 139.457 | 54.525 | 1.00 | 61.86 | B |
| ATOM | 1665 | C | VAL | B | 95 | 76.658 | 139.271 | 51.486 | 1.00 | 61.25 | B |
| ATOM | 1666 | O | VAL | B | 95 | 77.517 | 138.888 | 50.678 | 1.00 | 61.07 | B |
| ATOM | 1667 | N | TYR | B | 96 | 76.233 | 140.530 | 51.547 | 1.00 | 59.57 | B |
| ATOM | 1668 | CA | TYR | B | 96 | 76.788 | 141.588 | 50.707 | 1.00 | 57.73 | B |
| ATOM | 1669 | CB | TYR | B | 96 | 75.683 | 142.416 | 50.117 | 1.00 | 56.69 | B |
| ATOM | 1670 | CG | TYR | B | 96 | 75.143 | 141.778 | 48.881 | 1.00 | 56.49 | B |
| ATOM | 1671 | CD1 | TYR | B | 96 | 74.514 | 140.521 | 48.931 | 1.00 | 56.38 | B |
| ATOM | 1672 | CE1 | TYR | B | 96 | 74.060 | 139.898 | 47.773 | 1.00 | 55.40 | B |
| ATOM | 1673 | CD2 | TYR | B | 96 | 75.301 | 142.389 | 47.646 | 1.00 | 55.68 | B |
| ATOM | 1674 | CE2 | TYR | B | 96 | 74.853 | 141.769 | 46.488 | 1.00 | 55.94 | B |
| ATOM | 1675 | CZ | TYR | B | 96 | 74.236 | 140.530 | 46.559 | 1.00 | 54.68 | B |
| ATOM | 1676 | OH | TYR | B | 96 | 73.825 | 139.946 | 45.402 | 1.00 | 54.94 | B |
| ATOM | 1677 | C | TYR | B | 96 | 77.663 | 142.447 | 51.585 | 1.00 | 56.91 | B |
| ATOM | 1678 | O | TYR | B | 96 | 77.205 | 142.944 | 52.611 | 1.00 | 57.12 | B |
| ATOM | 1679 | N | LEU | B | 97 | 78.923 | 142.611 | 51.186 | 1.00 | 55.19 | B |
| ATOM | 1680 | CA | LEU | B | 97 | 79.858 | 143.361 | 51.987 | 1.00 | 53.46 | B |
| ATOM | 1681 | CB | LEU | B | 97 | 80.958 | 142.421 | 52.409 | 1.00 | 52.31 | B |
| ATOM | 1682 | CG | LEU | B | 97 | 80.455 | 141.184 | 53.115 | 1.00 | 51.89 | B |
| ATOM | 1683 | CD1 | LEU | B | 97 | 81.402 | 140.059 | 52.824 | 1.00 | 52.36 | B |
| ATOM | 1684 | CD2 | LEU | B | 97 | 80.378 | 141.419 | 54.624 | 1.00 | 51.37 | B |
| ATOM | 1685 | C | LEU | B | 97 | 80.449 | 144.605 | 51.303 | 1.00 | 52.87 | B |
| ATOM | 1686 | O | LEU | B | 97 | 80.459 | 144.720 | 50.075 | 1.00 | 53.20 | B |
| ATOM | 1687 | N | LYS | B | 98 | 80.928 | 145.535 | 52.119 | 1.00 | 51.29 | B |
| ATOM | 1688 | CA | LYS | B | 98 | 81.538 | 146.756 | 51.634 | 1.00 | 50.23 | B |
| ATOM | 1689 | CB | LYS | B | 98 | 80.520 | 147.888 | 51.685 | 1.00 | 52.43 | B |
| ATOM | 1690 | CG | LYS | B | 98 | 80.891 | 149.191 | 50.996 | 1.00 | 54.68 | B |
| ATOM | 1691 | CD | LYS | B | 98 | 79.834 | 150.268 | 51.334 | 1.00 | 56.71 | B |
| ATOM | 1692 | CE | LYS | B | 98 | 80.195 | 151.593 | 50.646 | 1.00 | 59.89 | B |
| ATOM | 1693 | NZ | LYS | B | 98 | 79.572 | 152.809 | 51.283 | 1.00 | 62.07 | B |
| ATOM | 1694 | C | LYS | B | 98 | 82.698 | 147.041 | 52.581 | 1.00 | 48.75 | B |
| ATOM | 1695 | O | LYS | B | 98 | 82.559 | 146.962 | 53.803 | 1.00 | 47.50 | B |
| ATOM | 1696 | N | ALA | B | 99 | 83.857 | 147.342 | 52.022 | 1.00 | 47.58 | B |
| ATOM | 1697 | CA | ALA | B | 99 | 85.009 | 147.625 | 52.878 | 1.00 | 45.82 | B |
| ATOM | 1698 | CB | ALA | B | 99 | 85.726 | 146.322 | 53.248 | 1.00 | 44.60 | B |
| ATOM | 1699 | C | ALA | B | 99 | 85.985 | 148.617 | 52.271 | 1.00 | 43.74 | B |
| ATOM | 1700 | O | ALA | B | 99 | 86.642 | 148.357 | 51.273 | 1.00 | 43.86 | B |
| ATOM | 1701 | N | PRO | B | 100 | 86.066 | 149.792 | 52.861 | 1.00 | 42.35 | B |
| ATOM | 1702 | CD | PRO | B | 100 | 85.265 | 150.269 | 53.998 | 1.00 | 41.96 | B |
| ATOM | 1703 | CA | PRO | B | 100 | 86.983 | 150.832 | 52.372 | 1.00 | 41.79 | B |
| ATOM | 1704 | CB | PRO | B | 100 | 86.386 | 152.110 | 52.941 | 1.00 | 41.25 | B |
| ATOM | 1705 | CG | PRO | B | 100 | 85.894 | 151.643 | 54.275 | 1.00 | 41.77 | B |
| ATOM | 1706 | C | PRO | B | 100 | 88.388 | 150.591 | 52.930 | 1.00 | 41.19 | B |
| ATOM | 1707 | O | PRO | B | 100 | 88.529 | 150.079 | 54.065 | 1.00 | 42.13 | B |
| ATOM | 1708 | N | MET | B | 101 | 89.403 | 150.961 | 52.138 | 1.00 | 39.97 | B |
| ATOM | 1709 | CA | MET | B | 101 | 90.819 | 150.830 | 52.517 | 1.00 | 38.92 | B |
| ATOM | 1710 | CB | MET | B | 101 | 91.275 | 149.368 | 52.444 | 1.00 | 37.82 | B |
| ATOM | 1711 | CG | MET | B | 101 | 91.661 | 148.911 | 51.037 | 1.00 | 38.13 | B |
| ATOM | 1712 | SD | MET | B | 101 | 91.028 | 147.242 | 50.716 | 1.00 | 42.53 | B |
| ATOM | 1713 | CE | MET | B | 101 | 92.349 | 146.532 | 50.111 | 1.00 | 43.61 | B |
| ATOM | 1714 | C | MET | B | 101 | 91.700 | 151.634 | 51.567 | 1.00 | 38.80 | B |
| ATOM | 1715 | O | MET | B | 101 | 91.252 | 152.116 | 50.534 | 1.00 | 38.87 | B |
| ATOM | 1716 | N | ILE | B | 102 | 92.968 | 151.795 | 51.923 | 1.00 | 38.72 | B |
| ATOM | 1717 | CA | ILE | B | 102 | 93.891 | 152.434 | 51.008 | 1.00 | 37.81 | B |
| ATOM | 1718 | CB | ILE | B | 102 | 94.813 | 153.463 | 51.649 | 1.00 | 37.11 | B |
| ATOM | 1719 | CG2 | ILE | B | 102 | 95.789 | 154.015 | 50.544 | 1.00 | 36.65 | B |
| ATOM | 1720 | CG1 | ILE | B | 102 | 93.966 | 154.613 | 52.190 | 1.00 | 37.57 | B |
| ATOM | 1721 | CD1 | ILE | B | 102 | 94.745 | 155.827 | 52.617 | 1.00 | 38.25 | B |
| ATOM | 1722 | C | ILE | B | 102 | 94.765 | 151.317 | 50.509 | 1.00 | 36.61 | B |
| ATOM | 1723 | O | ILE | B | 102 | 95.595 | 150.835 | 51.248 | 1.00 | 37.27 | B |
| ATOM | 1724 | N | LEU | B | 103 | 94.574 | 150.925 | 49.259 | 1.00 | 36.06 | B |
| ATOM | 1725 | CA | LEU | B | 103 | 95.338 | 149.838 | 48.616 | 1.00 | 36.16 | B |
| ATOM | 1726 | CB | LEU | B | 103 | 94.352 | 148.958 | 47.823 | 1.00 | 35.58 | B |
| ATOM | 1727 | CG | LEU | B | 103 | 94.844 | 147.783 | 47.003 | 1.00 | 35.65 | B |
| ATOM | 1728 | CD1 | LEU | B | 103 | 95.655 | 146.879 | 47.850 | 1.00 | 36.05 | B |
| ATOM | 1729 | CD2 | LEU | B | 103 | 93.639 | 147.040 | 46.463 | 1.00 | 35.38 | B |
| ATOM | 1730 | C | LEU | B | 103 | 96.489 | 150.338 | 47.707 | 1.00 | 35.57 | B |
| ATOM | 1731 | O | LEU | B | 103 | 96.246 | 150.959 | 46.681 | 1.00 | 35.97 | B |
| ATOM | 1732 | N | ASN | B | 104 | 97.731 | 150.066 | 48.085 | 1.00 | 34.28 | B |
| ATOM | 1733 | CA | ASN | B | 104 | 98.855 | 150.553 | 47.303 | 1.00 | 36.17 | B |
| ATOM | 1734 | CB | ASN | B | 104 | 98.972 | 149.819 | 45.962 | 1.00 | 34.92 | B |
| ATOM | 1735 | CG | ASN | B | 104 | 99.368 | 148.356 | 46.135 | 1.00 | 36.26 | B |
| ATOM | 1736 | OD1 | ASN | B | 104 | 99.938 | 147.958 | 47.171 | 1.00 | 36.00 | B |
| ATOM | 1737 | ND2 | ASN | B | 104 | 99.085 | 147.545 | 45.118 | 1.00 | 35.59 | B |
| ATOM | 1738 | C | ASN | B | 104 | 98.771 | 152.045 | 47.032 | 1.00 | 37.24 | B |
| ATOM | 1739 | O | ASN | B | 104 | 99.052 | 152.503 | 45.915 | 1.00 | 37.75 | B |
| ATOM | 1740 | N | GLY | B | 105 | 98.356 | 152.802 | 48.038 | 1.00 | 38.48 | B |
| ATOM | 1741 | CA | GLY | B | 105 | 98.288 | 154.237 | 47.864 | 1.00 | 39.80 | B |
| ATOM | 1742 | C | GLY | B | 105 | 97.085 | 154.809 | 47.160 | 1.00 | 40.39 | B |
| ATOM | 1743 | O | GLY | B | 105 | 97.089 | 156.005 | 46.841 | 1.00 | 42.15 | B |
| ATOM | 1744 | N | VAL | B | 106 | 96.058 | 154.021 | 46.890 | 1.00 | 39.73 | B |
| ATOM | 1745 | CA | VAL | B | 106 | 94.887 | 154.640 | 46.269 | 1.00 | 38.72 | B |
| ATOM | 1746 | CB | VAL | B | 106 | 94.755 | 154.258 | 44.791 | 1.00 | 39.21 | B |
| ATOM | 1747 | CG1 | VAL | B | 106 | 95.858 | 153.323 | 44.404 | 1.00 | 37.97 | B |
| ATOM | 1748 | CG2 | VAL | B | 106 | 93.390 | 153.684 | 44.506 | 1.00 | 38.76 | B |
| ATOM | 1749 | C | VAL | B | 106 | 93.658 | 154.254 | 47.044 | 1.00 | 38.46 | B |
| ATOM | 1750 | O | VAL | B | 106 | 93.485 | 153.112 | 47.431 | 1.00 | 38.56 | B |
| ATOM | 1751 | N | CYS | B | 107 | 92.807 | 155.214 | 47.331 | 1.00 | 39.54 | B |
| ATOM | 1752 | CA | CYS | B | 107 | 91.592 | 154.906 | 48.098 | 1.00 | 40.67 | B |
| ATOM | 1753 | CB | CYS | B | 107 | 90.937 | 156.201 | 48.584 | 1.00 | 41.28 | B |
| ATOM | 1754 | SG | CYS | B | 107 | 91.857 | 157.066 | 49.836 | 1.00 | 46.01 | B |
| ATOM | 1755 | C | CYS | B | 107 | 90.572 | 154.082 | 47.274 | 1.00 | 40.13 | B |
| ATOM | 1756 | O | CYS | B | 107 | 90.191 | 154.449 | 46.132 | 1.00 | 39.74 | B |
| ATOM | 1757 | N | VAL | B | 108 | 90.167 | 152.951 | 47.830 | 1.00 | 38.59 | B |
| ATOM | 1758 | CA | VAL | B | 108 | 89.215 | 152.155 | 47.125 | 1.00 | 40.12 | B |
| ATOM | 1759 | CB | VAL | B | 108 | 89.887 | 150.986 | 46.312 | 1.00 | 39.75 | B |
| ATOM | 1760 | CG1 | VAL | B | 108 | 90.839 | 151.532 | 45.262 | 1.00 | 39.25 | B |
| ATOM | 1761 | CG2 | VAL | B | 108 | 90.575 | 150.038 | 47.256 | 1.00 | 39.65 | B |
| ATOM | 1762 | C | VAL | B | 108 | 88.207 | 151.549 | 48.089 | 1.00 | 41.45 | B |
| ATOM | 1763 | O | VAL | B | 108 | 88.349 | 151.637 | 49.308 | 1.00 | 42.69 | B |
| ATOM | 1764 | N | ILE | B | 109 | 87.172 | 150.951 | 47.523 | 1.00 | 41.65 | B |
| ATOM | 1765 | CA | ILE | B | 109 | 86.190 | 150.283 | 48.299 | 1.00 | 42.07 | B |
| ATOM | 1766 | CB | ILE | B | 109 | 84.852 | 151.032 | 48.264 | 1.00 | 42.21 | B |
| ATOM | 1767 | CG2 | ILE | B | 109 | 83.786 | 150.251 | 49.063 | 1.00 | 41.33 | B |
| ATOM | 1768 | CG1 | ILE | B | 109 | 85.044 | 152.405 | 48.904 | 1.00 | 43.80 | B |
| ATOM | 1769 | CD1 | ILE | B | 109 | 83.809 | 153.340 | 48.841 | 1.00 | 44.20 | B |
| ATOM | 1770 | C | ILE | B | 109 | 86.082 | 148.917 | 47.623 | 1.00 | 42.66 | B |
| ATOM | 1771 | O | ILE | B | 109 | 86.041 | 148.830 | 46.392 | 1.00 | 41.53 | B |
| ATOM | 1772 | N | TRP | B | 110 | 86.080 | 147.878 | 48.454 | 1.00 | 43.13 | B |
| ATOM | 1773 | CA | TRP | B | 110 | 85.970 | 146.497 | 48.043 | 1.00 | 44.85 | B |
| ATOM | 1774 | CB | TRP | B | 110 | 86.826 | 145.606 | 48.963 | 1.00 | 46.13 | B |
| ATOM | 1775 | CG | TRP | B | 110 | 86.782 | 144.146 | 48.589 | 1.00 | 47.60 | B |
| ATOM | 1776 | CD2 | TRP | B | 110 | 85.778 | 143.173 | 48.949 | 1.00 | 47.94 | B |
| ATOM | 1777 | CE2 | TRP | B | 110 | 86.116 | 141.970 | 48.302 | 1.00 | 47.68 | B |
| ATOM | 1778 | CE3 | TRP | B | 110 | 84.635 | 143.210 | 49.745 | 1.00 | 48.96 | B |
| ATOM | 1779 | CD1 | TRP | B | 110 | 87.644 | 143.510 | 47.777 | 1.00 | 47.75 | B |
| ATOM | 1780 | NE1 | TRP | B | 110 | 87.262 | 142.210 | 47.595 | 1.00 | 48.55 | B |
| ATOM | 1781 | CZ2 | TRP | B | 110 | 85.360 | 140.799 | 48.414 | 1.00 | 47.78 | B |
| ATOM | 1782 | CZ3 | TRP | B | 110 | 83.862 | 142.034 | 49.863 | 1.00 | 50.70 | B |
| ATOM | 1783 | CH2 | TRP | B | 110 | 84.241 | 140.839 | 49.191 | 1.00 | 49.96 | B |
| ATOM | 1784 | C | TRP | B | 110 | 84.487 | 146.153 | 48.252 | 1.00 | 45.98 | B |
| ATOM | 1785 | O | TRP | B | 110 | 83.956 | 146.305 | 49.351 | 1.00 | 44.90 | B |
| ATOM | 1786 | N | LYS | B | 111 | 83.839 | 145.688 | 47.198 | 1.00 | 47.27 | B |
| ATOM | 1787 | CA | LYS | B | 111 | 82.429 | 145.322 | 47.265 | 1.00 | 49.19 | B |
| ATOM | 1788 | CB | LYS | B | 111 | 81.583 | 146.271 | 46.433 | 1.00 | 51.04 | B |
| ATOM | 1789 | CG | LYS | B | 111 | 81.506 | 147.711 | 46.847 | 1.00 | 54.53 | B |
| ATOM | 1790 | CD | LYS | B | 111 | 80.629 | 148.412 | 45.785 | 1.00 | 57.93 | B |
| ATOM | 1791 | CE | LYS | B | 111 | 80.281 | 149.877 | 46.107 | 1.00 | 60.45 | B |
| ATOM | 1792 | NZ | LYS | B | 111 | 79.187 | 150.333 | 45.174 | 1.00 | 61.82 | B |
| ATOM | 1793 | C | LYS | B | 111 | 82.221 | 143.944 | 46.661 | 1.00 | 49.11 | B |
| ATOM | 1794 | O | LYS | B | 111 | 82.626 | 143.694 | 45.522 | 1.00 | 49.83 | B |
| ATOM | 1795 | N | GLY | B | 112 | 81.569 | 143.061 | 47.393 | 1.00 | 48.46 | B |
| ATOM | 1796 | CA | GLY | B | 112 | 81.322 | 141.749 | 46.854 | 1.00 | 49.50 | B |
| ATOM | 1797 | C | GLY | B | 112 | 80.326 | 141.033 | 47.705 | 1.00 | 50.70 | B |
| ATOM | 1798 | O | GLY | B | 112 | 79.905 | 141.551 | 48.741 | 1.00 | 51.73 | B |
| ATOM | 1799 | N | TRP | B | 113 | 79.935 | 139.846 | 47.263 | 1.00 | 51.49 | B |
| ATOM | 1800 | CA | TRP | B | 113 | 78.980 | 139.022 | 48.004 | 1.00 | 52.41 | B |
| ATOM | 1801 | CB | TRP | B | 113 | 77.598 | 138.981 | 47.313 | 1.00 | 51.09 | B |
| ATOM | 1802 | CG | TRP | B | 113 | 77.590 | 138.452 | 45.920 | 1.00 | 49.59 | B |
| ATOM | 1803 | CD2 | TRP | B | 113 | 77.613 | 137.074 | 45.534 | 1.00 | 48.84 | B |
| ATOM | 1804 | CE2 | TRP | B | 113 | 77.636 | 137.033 | 44.126 | 1.00 | 48.77 | B |
| ATOM | 1805 | CE3 | TRP | B | 113 | 77.623 | 135.863 | 46.250 | 1.00 | 49.60 | B |
| ATOM | 1806 | CD1 | TRP | B | 113 | 77.597 | 139.179 | 44.757 | 1.00 | 49.20 | B |
| ATOM | 1807 | NE1 | TRP | B | 113 | 77.621 | 138.328 | 43.674 | 1.00 | 49.63 | B |
| ATOM | 1808 | CZ2 | TRP | B | 113 | 77.674 | 135.832 | 43.414 | 1.00 | 49.30 | B |
| ATOM | 1809 | CZ3 | TRP | B | 113 | 77.658 | 134.651 | 45.531 | 1.00 | 49.00 | B |
| ATOM | 1810 | CH2 | TRP | B | 113 | 77.684 | 134.653 | 44.139 | 1.00 | 49.09 | B |
| ATOM | 1811 | C | TRP | B | 113 | 79.502 | 137.602 | 48.182 | 1.00 | 53.77 | B |
| ATOM | 1812 | O | TRP | B | 113 | 80.332 | 137.099 | 47.420 | 1.00 | 52.39 | B |
| ATOM | 1813 | N | ILE | B | 114 | 79.004 | 136.951 | 49.216 | 1.00 | 56.21 | B |
| ATOM | 1814 | CA | ILE | B | 114 | 79.439 | 135.610 | 49.508 | 1.00 | 58.06 | B |
| ATOM | 1815 | CB | ILE | B | 114 | 80.522 | 135.651 | 50.600 | 1.00 | 57.92 | B |
| ATOM | 1816 | CG2 | ILE | B | 114 | 80.572 | 134.366 | 51.403 | 1.00 | 56.80 | B |
| ATOM | 1811 | CG1 | ILE | B | 114 | 81.864 | 135.944 | 49.936 | 1.00 | 57.77 | B |
| ATOM | 1818 | CD1 | ILE | B | 114 | 82.859 | 136.425 | 50.914 | 1.00 | 58.84 | B |
| ATOM | 1819 | C | ILE | B | 114 | 78.278 | 134.728 | 49.906 | 1.00 | 59.82 | B |
| ATOM | 1820 | O | ILE | B | 114 | 77.421 | 135.108 | 50.687 | 1.00 | 59.21 | B |
| ATOM | 1821 | N | ASP | B | 115 | 78.247 | 133.554 | 49.294 | 1.00 | 62.01 | B |
| ATOM | 1822 | CA | ASP | B | 115 | 77.240 | 132.535 | 49.550 | 1.00 | 64.00 | B |
| ATOM | 1823 | CB | ASP | B | 115 | 77.424 | 131.425 | 48.511 | 1.00 | 64.46 | B |
| ATOM | 1824 | CG | ASP | B | 115 | 76.533 | 130.197 | 48.749 | 1.00 | 64.55 | B |
| ATOM | 1825 | OD1 | ASP | B | 115 | 76.384 | 129.715 | 49.904 | 1.00 | 63.91 | B |
| ATOM | 1826 | OD2 | ASP | B | 115 | 76.010 | 129.694 | 47.729 | 1.00 | 64.87 | B |
| ATOM | 1827 | C | ASP | B | 115 | 77.548 | 131.991 | 50.952 | 1.00 | 64.47 | B |
| ATOM | 1828 | O | ASP | B | 115 | 78.569 | 131.328 | 51.133 | 1.00 | 65.31 | B |
| ATOM | 1829 | N | LEU | B | 116 | 16.678 | 132.235 | 51.928 | 1.00 | 64.86 | B |
| ATOM | 1830 | CA | LEU | B | 116 | 76.950 | 131.767 | 53.282 | 1.00 | 65.70 | B |
| ATOM | 1831 | CB | LEU | B | 116 | 75.866 | 132.260 | 54.222 | 1.00 | 65.57 | B |
| ATOM | 1832 | CG | LEU | B | 116 | 75.708 | 133.774 | 54.239 | 1.00 | 65.36 | B |
| ATOM | 1833 | CD1 | LEU | B | 116 | 74.294 | 134.115 | 54.594 | 1.00 | 64.41 | B |
| ATOM | 1834 | CD2 | LEU | B | 116 | 76.692 | 134.380 | 55.209 | 1.00 | 64.06 | B |
| ATOM | 1835 | C | LEU | B | 116 | 77.098 | 130.261 | 53.404 | 1.00 | 66.76 | B |
| ATOM | 1836 | O | LEU | B | 116 | 77.437 | 129.746 | 54.473 | 1.00 | 67.42 | B |
| ATOM | 1837 | N | GLN | B | 117 | 76.864 | 129.550 | 52.310 | 1.00 | 67.94 | B |
| ATOM | 1838 | CA | GLN | B | 117 | 76.989 | 128.101 | 52.334 | 1.00 | 69.00 | B |
| ATOM | 1839 | CB | GLN | B | 117 | 75.719 | 127.459 | 51.765 | 1.00 | 71.13 | B |
| ATOM | 1840 | CG | GLN | B | 117 | 75.596 | 125.961 | 52.116 | 1.00 | 74.75 | B |
| ATOM | 1841 | CD | GLN | B | 117 | 76.158 | 125.025 | 51.031 | 1.00 | 76.89 | B |
| ATOM | 1842 | OE1 | GLN | B | 117 | 75.404 | 124.551 | 50.157 | 1.00 | 78.35 | B |
| ATOM | 1843 | NE2 | GLN | B | 117 | 77.480 | 124.763 | 51.074 | 1.00 | 77.05 | B |
| ATOM | 1844 | C | GLN | B | 117 | 78.235 | 127.585 | 51.594 | 1.00 | 68.08 | B |
| ATOM | 1845 | O | GLN | B | 117 | 78.962 | 126.744 | 52.118 | 1.00 | 67.38 | B |
| ATOM | 1846 | N | ARG | B | 118 | 78.474 | 128.057 | 50.372 | 1.00 | 67.46 | B |
| ATOM | 1847 | CA | ARG | B | 118 | 79.676 | 127.631 | 49.640 | 1.00 | 66.55 | B |
| ATOM | 1848 | CB | ARG | B | 118 | 79.524 | 127.912 | 48.148 | 1.00 | 66.70 | B |
| ATOM | 1849 | CG | ARG | B | 118 | 78.148 | 127.671 | 47.582 | 1.00 | 67.77 | B |
| ATOM | 1850 | CD | ARG | B | 118 | 78.197 | 127.942 | 46.085 | 1.00 | 68.99 | B |
| ATOM | 1851 | NE | ARG | B | 118 | 79.289 | 127.151 | 45.535 | 1.00 | 71.45 | B |
| ATOM | 1852 | CZ | ARG | B | 118 | 79.680 | 127.143 | 44.260 | 1.00 | 73.49 | B |
| ATOM | 1853 | NH1 | ARG | B | 118 | 79.057 | 127.901 | 43.357 | 1.00 | 73.34 | B |
| ATOM | 1854 | NH2 | ARG | B | 118 | 80.718 | 126.374 | 43.888 | 1.00 | 73.72 | B |
| ATOM | 1855 | C | ARG | B | 118 | 80.935 | 128.401 | 50.184 | 1.00 | 65.39 | B |
| ATOM | 1856 | O | ARG | B | 118 | 82.065 | 127.885 | 50.188 | 1.00 | 65.15 | B |
| ATOM | 1857 | N | LEU | B | 119 | 80.720 | 129.624 | 50.666 | 1.00 | 62.91 | B |
| ATOM | 1858 | CA | LEU | B | 119 | 81.795 | 130.443 | 51.177 | 1.00 | 60.48 | B |
| ATOM | 1859 | CB | LEU | B | 119 | 82.643 | 129.622 | 52.147 | 1.00 | 59.64 | B |
| ATOM | 1860 | CG | LEU | B | 119 | 81.924 | 129.406 | 53.477 | 1.00 | 59.86 | B |
| ATOM | 1861 | CD1 | LEU | B | 119 | 82.858 | 128.733 | 54.459 | 1.00 | 59.89 | B |
| ATOM | 1862 | CD2 | LEU | B | 119 | 81.451 | 130.749 | 54.050 | 1.00 | 58.97 | B |
| ATOM | 1863 | C | LEU | B | 119 | 82.657 | 131.074 | 50.068 | 1.00 | 59.46 | B |
| ATOM | 1864 | O | LEU | B | 119 | 93.798 | 131.423 | 50.300 | 1.00 | 59.42 | B |
| ATOM | 1865 | N | ASP | B | 120 | 82.108 | 131.199 | 48.862 | 1.00 | 58.51 | B |
| ATOM | 1866 | CA | ASP | B | 120 | 82.800 | 131.834 | 47.730 | 1.00 | 58.11 | B |
| ATOM | 1867 | CB | ASP | B | 120 | 83.237 | 130.796 | 46.696 | 1.00 | 58.13 | B |
| ATOM | 1868 | CG | ASP | B | 120 | 82.076 | 130.065 | 46.048 | 1.00 | 58.80 | B |
| ATOM | 1869 | OD1 | ASP | B | 120 | 80.895 | 130.293 | 46.409 | 1.00 | 59.60 | B |
| ATOM | 1870 | OD2 | ASP | B | 120 | 82.354 | 129.241 | 45.157 | 1.00 | 58.20 | B |
| ATOM | 1871 | C | ASP | B | 120 | 81.848 | 132.861 | 47.088 | 1.00 | 58.01 | B |
| ATOM | 1872 | O | ASP | B | 120 | 80.714 | 133.033 | 47.555 | 1.00 | 58.16 | B |
| ATOM | 1873 | N | GLY | B | 121 | 82.277 | 133.557 | 46.043 | 1.00 | 56.92 | B |
| ATOM | 1874 | CA | GLY | B | 121 | 81.367 | 134.517 | 45.465 | 1.00 | 56.31 | B |
| ATOM | 1875 | C | GLY | B | 121 | 81.911 | 135.377 | 44.356 | 1.00 | 56.34 | B |
| ATOM | 1876 | O | GLY | B | 121 | 82.574 | 134.903 | 43.442 | 1.00 | 56.37 | B |
| ATOM | 1877 | N | MET | B | 122 | 81.567 | 136.655 | 44.412 | 1.00 | 56.20 | B |
| ATOM | 1878 | CA | MET | B | 122 | 82.011 | 137.633 | 43.424 | 1.00 | 55.62 | B |
| ATOM | 1879 | CB | MET | B | 122 | 80.975 | 137.817 | 42.314 | 1.00 | 54.51 | B |
| ATOM | 1880 | CG | MET | B | 122 | 80.648 | 136.518 | 41.551 | 1.00 | 54.38 | B |
| ATOM | 1881 | SD | MET | B | 122 | 82.057 | 135.772 | 40.582 | 1.00 | 53.79 | B |
| ATOM | 1882 | CE | MET | B | 122 | 81.878 | 136.724 | 39.036 | 1.00 | 53.41 | B |
| ATOM | 1883 | C | MET | B | 122 | 82.242 | 138.965 | 44.115 | 1.00 | 55.44 | B |
| ATOM | 1884 | O | MET | B | 122 | 81.578 | 139.328 | 45.105 | 1.00 | 55.00 | B |
| ATOM | 1885 | N | GLY | B | 123 | 83.196 | 139.703 | 43.581 | 1.00 | 54.81 | B |
| ATOM | 1886 | CA | GLY | B | 123 | 83.519 | 140.987 | 44.168 | 1.00 | 53.07 | B |
| ATOM | 1887 | C | GLY | B | 123 | 84.509 | 141.712 | 43.307 | 1.00 | 51.73 | B |
| ATOM | 1888 | O | GLY | B | 123 | 85.087 | 141.157 | 42.368 | 1.00 | 51.04 | B |
| ATOM | 1889 | N | CYS | B | 124 | 84.713 | 142.971 | 43.624 | 1.00 | 50.71 | B |
| ATOM | 1890 | CA | CYS | B | 124 | 85.629 | 143.730 | 42.835 | 1.00 | 50.39 | B |
| ATOM | 1891 | CB | CYS | B | 124 | 84.955 | 144.175 | 41.543 | 1.00 | 52.10 | B |
| ATOM | 1892 | SG | CYS | B | 124 | 83.623 | 145.344 | 41.845 | 1.00 | 54.77 | B |
| ATOM | 1893 | C | CYS | B | 124 | 86.035 | 144.915 | 43.624 | 1.00 | 49.39 | B |
| ATOM | 1894 | O | CYS | B | 124 | 85.544 | 145.155 | 44.713 | 1.00 | 49.67 | B |
| ATOM | 1895 | N | LEU | B | 125 | 86.928 | 145.680 | 43.038 | 1.00 | 49.76 | B |
| ATOM | 1896 | CA | LEU | B | 125 | 87.463 | 146.856 | 43.674 | 1.00 | 49.91 | B |
| ATOM | 1897 | CB | LEU | B | 125 | 88.986 | 146.885 | 43.459 | 1.00 | 47.74 | B |
| ATOM | 1898 | CG | LEU | B | 125 | 89.899 | 146.713 | 44.664 | 1.00 | 47.66 | B |
| ATOM | 1899 | CD1 | LEU | B | 125 | 89.335 | 145.662 | 45.626 | 1.00 | 45.69 | B |
| ATOM | 1900 | CD2 | LEU | B | 125 | 91.296 | 146.367 | 44.177 | 1.00 | 45.44 | B |
| ATOM | 1901 | C | LEU | B | 125 | 86.786 | 147.996 | 42.970 | 1.00 | 50.57 | B |
| ATOM | 1902 | O | LEU | B | 125 | 86.539 | 147.908 | 41.784 | 1.00 | 51.14 | B |
| ATOM | 1903 | N | GLU | B | 126 | 86.476 | 149.051 | 43.713 | 1.00 | 51.74 | B |
| ATOM | 1904 | CA | GLU | B | 126 | 85.836 | 150.259 | 43.183 | 1.00 | 52.16 | B |
| ATOM | 1905 | CB | GLU | B | 126 | 84.434 | 150.336 | 43.738 | 1.00 | 52.81 | B |
| ATOM | 1906 | CG | GLU | B | 126 | 83.394 | 150.756 | 42.770 | 1.00 | 56.52 | B |
| ATOM | 1907 | CD | GLU | B | 126 | 82.035 | 150.804 | 43.422 | 1.00 | 59.48 | B |
| ATOM | 1908 | OE1 | GLU | B | 126 | 81.908 | 151.609 | 44.380 | 1.00 | 60.69 | B |
| ATOM | 1909 | OE2 | GLU | B | 126 | 81.109 | 150.052 | 42.993 | 1.00 | 59.66 | B |
| ATOM | 1910 | C | GLU | B | 126 | 86.631 | 151.513 | 43.627 | 1.00 | 52.15 | B |
| ATOM | 1911 | O | GLU | B | 126 | 87.000 | 151.651 | 44.787 | 1.00 | 51.08 | B |
| ATOM | 1912 | N | PHE | B | 127 | 86.911 | 152.427 | 42.715 | 1.00 | 52.43 | B |
| ATOM | 1913 | CA | PHE | B | 127 | 87.669 | 153.619 | 43.114 | 1.00 | 52.71 | B |
| ATOM | 1914 | CB | PHE | B | 127 | 88.017 | 154.523 | 41.913 | 1.00 | 51.92 | B |
| ATOM | 1915 | CG | PHE | B | 127 | 88.797 | 155.744 | 42.290 | 1.00 | 52.10 | B |
| ATOM | 1916 | CD1 | PHE | B | 127 | 90.066 | 155.640 | 42.847 | 1.00 | 52.50 | B |
| ATOM | 1917 | CD2 | PHE | B | 127 | 88.309 | 157.013 | 42.016 | 1.00 | 52.93 | B |
| ATOM | 1918 | CE1 | PHE | B | 127 | 90.851 | 156.788 | 43.110 | 1.00 | 52.64 | B |
| ATOM | 1919 | CE2 | PHE | B | 127 | 89.090 | 158.177 | 42.282 | 1.00 | 53.06 | B |
| ATOM | 1920 | CZ | PHE | B | 127 | 90.360 | 158.053 | 42.823 | 1.00 | 52.57 | B |
| ATOM | 1921 | C | PHE | B | 127 | 86.828 | 154.418 | 44.049 | 1.00 | 52.37 | B |
| ATOM | 1922 | O | PHE | B | 127 | 85.698 | 154.707 | 43.720 | 1.00 | 52.87 | B |
| ATOM | 1923 | N | ASP | B | 128 | 87.360 | 154.802 | 45.199 | 1.00 | 52.71 | B |
| ATOM | 1924 | CA | ASP | B | 128 | 86.576 | 155.595 | 46.150 | 1.00 | 53.37 | B |
| ATOM | 1925 | CB | ASP | B | 128 | 86.862 | 155.090 | 47.560 | 1.00 | 53.50 | B |
| ATOM | 1926 | CG | ASP | B | 128 | 86.042 | 155.816 | 48.615 | 1.00 | 52.97 | B |
| ATOM | 1927 | OD1 | ASP | B | 128 | 85.537 | 156.922 | 48.303 | 1.00 | 53.84 | B |
| ATOM | 1928 | OD2 | ASP | B | 128 | 85.915 | 155.280 | 49.741 | 1.00 | 51.55 | B |
| ATOM | 1929 | C | ASP | B | 128 | 86.950 | 157.080 | 46.046 | 1.00 | 54.64 | B |
| ATOM | 1930 | O | ASP | B | 128 | 87.715 | 157.608 | 46.865 | 1.00 | 53.70 | B |
| ATOM | 1931 | N | GLU | B | 129 | 86.412 | 157.791 | 45.073 | 1.00 | 57.08 | B |
| ATOM | 1932 | CA | GLU | B | 129 | 86.834 | 159.188 | 44.920 | 1.00 | 59.54 | B |
| ATOM | 1933 | CB | GLU | B | 129 | 86.297 | 159.743 | 43.622 | 1.00 | 61.77 | B |
| ATOM | 1934 | CG | GLU | B | 129 | 85.280 | 160.805 | 43.799 | 1.00 | 66.59 | B |
| ATOM | 1935 | CD | GLU | B | 129 | 84.815 | 161.285 | 42.455 | 1.00 | 70.37 | B |
| ATOM | 1936 | OE1 | GLU | B | 129 | 83.720 | 161.949 | 42.387 | 1.00 | 72.00 | B |
| ATOM | 1937 | OE2 | GLU | B | 129 | 85.572 | 160.978 | 41.466 | 1.00 | 71.39 | B |
| ATOM | 1938 | C | GLU | B | 129 | 86.546 | 160.173 | 46.056 | 1.00 | 59.43 | B |
| ATOM | 1939 | O | GLU | B | 129 | 87.289 | 161.154 | 46.247 | 1.00 | 58.28 | B |
| ATOM | 1940 | N | GLU | B | 130 | 85.467 | 159.950 | 46.796 | 1.00 | 59.34 | B |
| ATOM | 1941 | CA | GLU | B | 130 | 85.221 | 160.855 | 47.889 | 1.00 | 60.50 | B |
| ATOM | 1942 | CB | GLU | B | 130 | 83.984 | 160.445 | 48.642 | 1.00 | 61.68 | B |
| ATOM | 1943 | CG | GLU | B | 130 | 83.358 | 161.613 | 49.361 | 1.00 | 65.38 | B |
| ATOM | 1944 | CD | GLU | B | 130 | 82.721 | 161.209 | 50.713 | 1.00 | 68.30 | B |
| ATOM | 1945 | OE1 | GLU | B | 130 | 83.413 | 161.428 | 51.759 | 1.00 | 68.99 | B |
| ATOM | 1946 | OE2 | GLU | B | 130 | 81.551 | 160.664 | 50.727 | 1.00 | 69.49 | B |
| ATOM | 1947 | C | GLU | B | 130 | 86.462 | 160.786 | 48.814 | 1.00 | 60.71 | B |
| ATOM | 1948 | O | GLU | B | 130 | 87.159 | 161.782 | 48.984 | 1.00 | 61.04 | B |
| ATOM | 1949 | N | ARG | B | 131 | 86.740 | 159.606 | 49.385 | 1.00 | 60.41 | B |
| ATOM | 1950 | CA | ARG | B | 131 | 87.897 | 159.383 | 50.260 | 1.00 | 59.68 | B |
| ATOM | 1951 | CB | ARG | B | 131 | 88.117 | 157.898 | 50.521 | 1.00 | 60.38 | B |
| ATOM | 1952 | CG | ARG | B | 131 | 87.133 | 157.231 | 51.402 | 1.00 | 62.32 | B |
| ATOM | 1953 | CD | ARG | B | 131 | 87.309 | 157.702 | 52.770 | 1.00 | 65.35 | B |
| ATOM | 1954 | NE | ARG | B | 131 | 88.680 | 157.491 | 53.227 | 1.00 | 69.78 | B |
| ATOM | 1955 | CZ | ARG | B | 131 | 89.136 | 157.823 | 54.450 | 1.00 | 71.27 | B |
| ATOM | 1956 | NH1 | ARG | B | 131 | 88.321 | 158.393 | 55.368 | 1.00 | 71.32 | B |
| ATOM | 1957 | NH2 | ARG | B | 131 | 90.414 | 157.585 | 54.757 | 1.00 | 71.12 | B |
| ATOM | 1958 | C | ARG | B | 131 | 89.190 | 159.891 | 49.655 | 1.00 | 59.27 | B |
| ATOM | 1959 | O | ARG | B | 131 | 90.020 | 160.472 | 50.342 | 1.00 | 58.93 | B |
| ATOM | 1960 | N | ALA | B | 132 | 89.378 | 159.646 | 48.369 | 1.00 | 58.40 | B |
| ATOM | 1961 | CA | ALA | B | 132 | 90.611 | 160.057 | 47.755 | 1.00 | 58.76 | B |
| ATOM | 1962 | CB | ALA | B | 132 | 90.600 | 159.691 | 46.310 | 1.00 | 58.45 | B |
| ATOM | 1963 | C | ALA | B | 132 | 90.867 | 161.530 | 47.907 | 1.00 | 59.46 | B |
| ATOM | 1964 | O | ALA | B | 132 | 91.963 | 161.966 | 48.276 | 1.00 | 59.19 | B |
| ATOM | 1965 | N | GLN | B | 133 | 89.840 | 162.309 | 47.616 | 1.00 | 61.39 | B |
| ATOM | 1966 | CA | GLN | B | 133 | 89.965 | 163.761 | 47.662 | 1.00 | 62.64 | B |
| ATOM | 1967 | CB | GLN | B | 133 | 88.729 | 164.430 | 47.054 | 1.00 | 63.07 | B |
| ATOM | 1968 | CG | GLN | B | 133 | 88.633 | 164.196 | 45.572 | 1.00 | 64.53 | B |
| ATOM | 1969 | CD | GLN | B | 133 | 87.307 | 164.609 | 45.000 | 1.00 | 66.27 | B |
| ATOM | 1970 | OE1 | GLN | B | 133 | 87.107 | 164.597 | 43.767 | 1.00 | 66.89 | B |
| ATOM | 1971 | NE2 | GLN | B | 133 | 86.372 | 164.975 | 45.882 | 1.00 | 66.94 | B |
| ATOM | 1972 | C | GLN | B | 133 | 90.199 | 164.230 | 49.063 | 1.00 | 63.23 | B |
| ATOM | 1973 | O | GLN | B | 133 | 90.787 | 165.276 | 49.274 | 1.00 | 64.09 | B |
| ATOM | 1974 | N | GLN | B | 134 | 89.760 | 163.472 | 50.041 | 1.00 | 63.35 | B |
| ATOM | 1975 | CA | GLN | B | 134 | 90.019 | 163.949 | 51.359 | 1.00 | 64.37 | B |
| ATOM | 1976 | CB | GLN | B | 134 | 89.087 | 163.307 | 52.354 | 1.00 | 66.27 | B |
| ATOM | 1977 | CG | GLN | B | 134 | 87.621 | 163.532 | 52.051 | 1.00 | 69.95 | B |
| ATOM | 1978 | CD | GLN | B | 134 | 86.733 | 162.479 | 52.724 | 1.00 | 72.36 | B |
| ATOM | 1979 | OE1 | GLN | B | 134 | 86.822 | 161.274 | 52.429 | 1.00 | 73.33 | B |
| ATOM | 1980 | NE2 | GLN | B | 134 | 85.876 | 162.930 | 53.634 | 1.00 | 73.84 | B |
| ATOM | 1981 | C | GLN | B | 134 | 91.443 | 163.644 | 51.739 | 1.00 | 64.62 | B |
| ATOM | 1982 | O | GLN | B | 134 | 91.934 | 164.220 | 52.691 | 1.00 | 65.38 | B |
| ATOM | 1983 | N | GLN | B | 135 | 92.127 | 162.769 | 50.997 | 1.00 | 64.52 | B |
| ATOM | 1984 | CA | GLN | B | 135 | 93.507 | 162.374 | 51.353 | 1.00 | 64.18 | B |
| ATOM | 1985 | CB | GLN | B | 135 | 93.870 | 161.012 | 50.724 | 1.00 | 62.79 | B |
| ATOM | 1986 | CG | GLU | B | 135 | 93.416 | 159.752 | 51.506 | 1.00 | 60.42 | B |
| ATOM | 1987 | CD | GLU | B | 135 | 93.896 | 159.723 | 52.966 | 1.00 | 58.12 | B |
| ATOM | 1988 | OE1 | GLU | B | 135 | 95.042 | 160.126 | 53.240 | 1.00 | 55.61 | B |
| ATOM | 1989 | OE2 | GLU | B | 135 | 93.122 | 159.284 | 53.840 | 1.00 | 56.74 | B |
| ATOM | 1990 | C | GLU | B | 135 | 94.658 | 163.339 | 51.078 | 1.00 | 65.07 | B |
| ATOM | 1991 | O | GLU | B | 135 | 94.533 | 164.260 | 50.248 | 1.00 | 65.69 | B |
| ATOM | 1992 | OXT | GLU | B | 135 | 95.713 | 163.127 | 51.701 | 1.00 | 65.59 | B |
| ATOM | 1993 | CB | ALA | C | 54 | 31.509 | 107.021 | 95.339 | 0.00 | 78.16 | C |
| ATOM | 1994 | C | ALA | C | 54 | 29.299 | 106.939 | 94.157 | 0.00 | 78.20 | C |
| ATOM | 1995 | O | ALA | C | 54 | 28.414 | 106.423 | 93.487 | 0.00 | 78.20 | C |
| ATOM | 1996 | N | ALA | C | 54 | 30.800 | 104.980 | 94.133 | 0.00 | 78.16 | C |
| ATOM | 1997 | CA | ALA | C | 54 | 30.322 | 106.132 | 94.937 | 0.00 | 78.18 | C |
| ATOM | 1998 | N | ALA | C | 55 | 30.062 | 108.611 | 93.534 | 0.00 | 81.07 | C |
| ATOM | 1999 | CA | ALA | C | 55 | 29.060 | 109.320 | 92.771 | 0.00 | 81.37 | C |
| ATOM | 2000 | CB | ALA | C | 55 | 28.947 | 110.729 | 93.296 | 0.00 | 81.05 | C |
| ATOM | 2001 | C | ALA | C | 55 | 29.484 | 109.327 | 91.313 | 0.00 | 81.69 | C |
| ATOM | 2002 | O | ALA | C | 55 | 28.662 | 109.166 | 90.413 | 0.00 | 81.59 | C |
| ATOM | 2003 | N | ALA | C | 56 | 30.783 | 109.495 | 91.095 | 1.00 | 82.56 | C |
| ATOM | 2004 | CA | ALA | C | 56 | 31.343 | 109.551 | 89.735 | 1.00 | 83.07 | C |
| ATOM | 2005 | CB | ALA | C | 56 | 31.009 | 108.222 | 88.986 | 1.00 | 82.24 | C |
| ATOM | 2006 | C | ALA | C | 56 | 30.788 | 110.800 | 88.967 | 1.00 | 82.90 | C |
| ATOM | 2007 | O | ALA | C | 56 | 29.566 | 110.863 | 88.693 | 1.00 | 83.47 | C |
| ATOM | 2008 | N | ALA | C | 57 | 31.674 | 111.775 | 88.650 | 1.00 | 82.16 | C |
| ATOM | 2009 | CA | ALA | C | 57 | 31.287 | 113.024 | 87.944 | 1.00 | 81.05 | C |
| ATOM | 2010 | CB | ALA | C | 57 | 29.793 | 113.385 | 88.290 | 1.00 | 81.36 | C |
| ATOM | 2011 | C | ALA | C | 57 | 32.136 | 114.308 | 88.153 | 1.00 | 79.87 | C |
| ATOM | 2012 | O | ALA | C | 57 | 31.737 | 115.123 | 89.021 | 1.00 | 80.05 | C |
| ATOM | 2013 | N | HIS | C | 58 | 33.212 | 114.552 | 87.366 | 1.00 | 77.39 | C |
| ATOM | 2014 | CA | HIS | C | 58 | 34.019 | 115.791 | 87.582 | 1.00 | 74.53 | C |
| ATOM | 2015 | CB | HIS | C | 58 | 35.025 | 115.550 | 88.737 | 1.00 | 76.62 | C |
| ATOM | 2016 | CG | HIS | C | 58 | 36.435 | 115.207 | 88.288 | 1.00 | 80.21 | C |
| ATOM | 2017 | CD2 | HIS | C | 58 | 37.195 | 114.098 | 88.501 | 1.00 | 80.47 | C |
| ATOM | 2018 | ND1 | HIS | C | 58 | 37.250 | 116.096 | 87.595 | 1.00 | 80.57 | C |
| ATOM | 2019 | CE1 | HIS | C | 58 | 38.444 | 115.552 | 87.412 | 1.00 | 80.21 | C |
| ATOM | 2020 | NE2 | HIS | C | 58 | 38.440 | 114.343 | 87.950 | 1.00 | 80.90 | C |
| ATOM | 2021 | C | HIS | C | 58 | 34.777 | 116.579 | 86.461 | 1.00 | 71.07 | C |
| ATOM | 2022 | O | HIS | C | 58 | 35.011 | 117.792 | 86.622 | 1.00 | 71.47 | C |
| ATOM | 2023 | N | PRO | C | 59 | 35.208 | 115.920 | 85.350 | 1.00 | 67.00 | C |
| ATOM | 2024 | CD | PRO | C | 59 | 35.016 | 114.514 | 84.965 | 1.00 | 65.55 | C |
| ATOM | 2025 | CA | PRO | C | 59 | 35.928 | 116.624 | 84.280 | 1.00 | 63.29 | C |
| ATOM | 2026 | CB | PRO | C | 59 | 35.960 | 115.604 | 83.163 | 1.00 | 62.60 | C |
| ATOM | 2027 | CG | PRO | C | 59 | 36.026 | 114.375 | 83.869 | 1.00 | 63.84 | C |
| ATOM | 2028 | C | PRO | C | 59 | 35.302 | 117.937 | 83.807 | 1.00 | 60.69 | C |
| ATOM | 2029 | O | PRO | C | 59 | 36.038 | 118.880 | 83.512 | 1.00 | 60.29 | C |
| ATOM | 2030 | N | GLY | C | 60 | 33.967 | 117.977 | 83.711 | 1.00 | 57.36 | C |
| ATOM | 2031 | CA | GLY | C | 60 | 33.282 | 119.169 | 83.258 | 1.00 | 53.47 | C |
| ATOM | 2032 | C | GLY | C | 60 | 33.142 | 119.294 | 81.743 | 1.00 | 51.27 | C |
| ATOM | 2033 | O | GLY | C | 60 | 33.157 | 118.310 | 81.031 | 1.00 | 50.03 | C |
| ATOM | 2034 | N | GLU | C | 61 | 33.035 | 120.540 | 81.275 | 1.00 | 50.14 | C |
| ATOM | 2035 | CA | GLU | C | 61 | 32.852 | 120.918 | 79.877 | 1.00 | 47.89 | C |
| ATOM | 2036 | CB | GLU | C | 61 | 32.306 | 122.305 | 79.885 | 1.00 | 49.94 | C |
| ATOM | 2037 | CG | GLU | C | 61 | 32.318 | 122.897 | 78.557 | 1.00 | 55.41 | C |
| ATOM | 2038 | CD | GLU | C | 61 | 30.970 | 122.922 | 77.961 | 1.00 | 57.71 | C |
| ATOM | 2039 | OE1 | GLU | C | 61 | 30.471 | 121.838 | 77.565 | 1.00 | 58.70 | C |
| ATOM | 2040 | OE2 | GLU | C | 61 | 30.424 | 124.046 | 77.905 | 1.00 | 59.70 | C |
| ATOM | 2041 | C | GLU | C | 61 | 34.135 | 120.872 | 79.056 | 1.00 | 45.48 | C |
| ATOM | 2042 | O | GLU | C | 61 | 34.937 | 121.808 | 79.097 | 1.00 | 45.67 | C |
| ATOM | 2043 | N | LEU | C | 62 | 34.308 | 119.825 | 78.252 | 1.00 | 42.60 | C |
| ATOM | 2044 | CA | LEU | C | 62 | 35.582 | 119.632 | 77.528 | 1.00 | 38.99 | C |
| ATOM | 2045 | CB | LEU | C | 62 | 36.158 | 118.250 | 77.832 | 1.00 | 37.06 | C |
| ATOM | 2046 | CG | LEU | C | 62 | 36.306 | 117.825 | 79.296 | 1.00 | 36.72 | C |
| ATOM | 2047 | CD1 | LEU | C | 62 | 36.729 | 116.356 | 79.439 | 1.00 | 36.32 | C |
| ATOM | 2048 | CD2 | LEU | C | 62 | 37.334 | 118.750 | 79.945 | 1.00 | 36.02 | C |
| ATOM | 2049 | C | LEU | C | 62 | 35.519 | 119.740 | 76.046 | 1.00 | 38.45 | C |
| ATOM | 2050 | O | LEU | C | 62 | 34.498 | 119.420 | 75.451 | 1.00 | 39.35 | C |
| ATOM | 2051 | N | VAL | C | 63 | 36.623 | 120.166 | 75.443 | 1.00 | 36.28 | C |
| ATOM | 2052 | CA | VAL | C | 63 | 36.727 | 120.247 | 73.978 | 1.00 | 33.69 | C |
| ATOM | 2053 | CB | VAL | C | 63 | 36.810 | 121.707 | 73.496 | 1.00 | 32.02 | C |
| ATOM | 2054 | CG1 | VAL | C | 63 | 35.550 | 122.350 | 73.826 | 1.00 | 32.48 | C |
| ATOM | 2055 | CG2 | VAL | C | 63 | 37.939 | 122.441 | 74.147 | 1.00 | 30.77 | C |
| ATOM | 2056 | C | VAL | C | 63 | 37.960 | 119.455 | 73.513 | 1.00 | 32.66 | C |
| ATOM | 2057 | O | VAL | C | 63 | 38.876 | 119.227 | 74.292 | 1.00 | 33.76 | C |
| ATOM | 2058 | N | ARG | C | 64 | 37.974 | 118.966 | 72.281 | 1.00 | 31.73 | C |
| ATOM | 2059 | CA | ARG | C | 64 | 39.162 | 118.237 | 71.836 | 1.00 | 31.92 | C |
| ATOM | 2060 | CB | ARG | C | 64 | 38.900 | 117.444 | 70.521 | 1.00 | 30.66 | C |
| ATOM | 2061 | CG | ARG | C | 64 | 37.607 | 116.620 | 70.483 | 1.00 | 33.82 | C |
| ATOM | 2062 | CD | ARG | C | 64 | 37.519 | 115.457 | 69.432 | 1.00 | 32.94 | C |
| ATOM | 2063 | NE | ARG | C | 64 | 38.626 | 114.563 | 69.712 | 1.00 | 35.16 | C |
| ATOM | 2064 | CZ | ARG | C | 64 | 38.585 | 113.237 | 69.748 | 1.00 | 34.47 | C |
| ATOM | 2065 | NH1 | ARG | C | 64 | 37.436 | 112.578 | 69.507 | 1.00 | 32.41 | C |
| ATOM | 2066 | NH2 | ARG | C | 64 | 39.743 | 112.580 | 70.006 | 1.00 | 34.13 | C |
| ATOM | 2067 | C | ARG | C | 64 | 40.263 | 119.291 | 71.585 | 1.00 | 32.21 | C |
| ATOM | 2068 | O | ARG | C | 64 | 39.939 | 120.403 | 71.103 | 1.00 | 32.87 | C |
| ATOM | 2069 | N | THR | C | 65 | 41.528 | 118.983 | 71.907 | 1.00 | 31.54 | C |
| ATOM | 2070 | CA | THR | C | 65 | 42.652 | 119.908 | 71.648 | 1.00 | 32.05 | C |
| ATOM | 2071 | CB | THR | C | 65 | 43.821 | 119.681 | 72.604 | 1.00 | 31.98 | C |
| ATOM | 2072 | OG1 | THR | C | 65 | 44.454 | 118.425 | 72.284 | 1.00 | 32.02 | C |
| ATOM | 2073 | CG2 | THR | C | 65 | 43.349 | 119.654 | 74.018 | 1.00 | 30.75 | C |
| ATOM | 2074 | C | THR | C | 65 | 43.169 | 119.535 | 70.261 | 1.00 | 32.29 | C |
| ATOM | 2075 | O | THR | C | 65 | 42.634 | 118.614 | 69.670 | 1.00 | 32.59 | C |
| ATOM | 2076 | N | ASP | C | 66 | 44.179 | 120.231 | 69.732 | 1.00 | 32.65 | C |
| ATOM | 2077 | CA | ASP | C | 66 | 44.750 | 119.818 | 68.431 | 1.00 | 32.97 | C |
| ATOM | 2078 | CB | ASP | C | 66 | 45.731 | 220.864 | 67.880 | 1.00 | 33.66 | C |
| ATOM | 2079 | CG | ASP | C | 66 | 45.077 | 122.219 | 67.724 | 1.00 | 36.22 | C |
| ATOM | 2080 | OD1 | ASP | C | 66 | 43.830 | 122.226 | 67.480 | 1.00 | 37.77 | C |
| ATOM | 2081 | OD2 | ASP | C | 66 | 45.797 | 123.258 | 67.856 | 1.00 | 33.68 | C |
| ATOM | 2082 | C | ASP | C | 66 | 45.506 | 118.463 | 68.532 | 1.00 | 32.11 | C |
| ATOM | 2083 | O | ASP | C | 66 | 46.089 | 117.975 | 67.553 | 1.00 | 32.41 | C |
| ATOM | 2084 | N | SER | C | 67 | 45.534 | 117.861 | 69.702 | 1.00 | 30.28 | C |
| ATOM | 2085 | CA | SER | C | 67 | 46.226 | 116.615 | 69.784 | 1.00 | 29.91 | C |
| ATOM | 2086 | CB | SER | C | 67 | 47.160 | 116.620 | 70.972 | 1.00 | 29.65 | C |
| ATOM | 2087 | OG | SER | C | 67 | 47.347 | 115.297 | 71.438 | 1.00 | 27.11 | C |
| ATOM | 2088 | C | SER | C | 67 | 45.171 | 115.542 | 69.973 | 1.00 | 30.94 | C |
| ATOM | 2089 | O | SER | C | 67 | 44.142 | 115.767 | 70.585 | 1.00 | 30.50 | C |
| ATOM | 2090 | N | PRO | C | 68 | 45.414 | 114.355 | 69.436 | 1.00 | 30.65 | C |
| ATOM | 2091 | CD | PRO | C | 68 | 46.460 | 114.138 | 68.427 | 1.00 | 30.13 | C |
| ATOM | 2092 | CA | PRO | C | 68 | 44.496 | 113.206 | 69.530 | 1.00 | 29.30 | C |
| ATOM | 2093 | CB | PRO | C | 68 | 44.919 | 112.320 | 68.369 | 1.00 | 28.56 | C |
| ATOM | 2094 | CG | PRO | C | 68 | 46.419 | 112.611 | 68.259 | 1.00 | 29.48 | C |
| ATOM | 2095 | C | PRO | C | 68 | 44.610 | 112.488 | 70.855 | 1.00 | 28.82 | C |
| ATOM | 2096 | O | PRO | C | 68 | 43.973 | 111.482 | 71.096 | 1.00 | 30.03 | C |
| ATOM | 2097 | N | ASN | C | 69 | 45.462 | 112.977 | 71.717 | 1.00 | 27.74 | C |
| ATOM | 2098 | CA | ASN | C | 69 | 45.589 | 112.323 | 73.025 | 1.00 | 27.54 | C |
| ATOM | 2099 | CB | ASN | C | 69 | 47.042 | 111.914 | 73.242 | 1.00 | 27.65 | C |
| ATOM | 2100 | CG | ASN | C | 69 | 47.158 | 110.661 | 74.057 | 1.00 | 30.52 | C |
| ATOM | 2101 | OD1 | ASN | C | 69 | 46.236 | 109.844 | 74.064 | 1.00 | 30.86 | C |
| ATOM | 2102 | ND2 | ASN | C | 69 | 48.301 | 110.472 | 74.731 | 1.00 | 31.95 | C |
| ATOM | 2103 | C | ASN | C | 69 | 45.119 | 113.212 | 74.208 | 1.00 | 26.77 | C |
| ATOM | 2104 | O | ASN | C | 69 | 45.305 | 112.845 | 75.336 | 1.00 | 24.81 | C |
| ATOM | 2105 | N | PHE | C | 70 | 44.528 | 114.383 | 73.925 | 1.00 | 27.89 | C |
| ATOM | 2106 | CA | PHE | C | 70 | 44.113 | 115.264 | 74.987 | 1.00 | 27.73 | C |
| ATOM | 2107 | CB | PHE | C | 70 | 45.245 | 116.239 | 75.342 | 1.00 | 26.91 | C |
| ATOM | 2108 | CG | PHE | C | 70 | 46.521 | 115.569 | 75.715 | 1.00 | 24.40 | C |
| ATOM | 2109 | CD1 | PHE | C | 70 | 47.444 | 115.311 | 74.769 | 1.00 | 21.53 | C |
| ATOM | 2110 | CD2 | PHE | C | 70 | 46.740 | 115.119 | 77.033 | 1.00 | 27.10 | C |
| ATOM | 2111 | CE1 | PHE | C | 70 | 48.588 | 114.602 | 75.086 | 1.00 | 25.09 | C |
| ATOM | 2112 | CE2 | PHE | C | 70 | 47.875 | 114.408 | 77.392 | 1.00 | 24.83 | C |
| ATOM | 2113 | CZ | PHE | C | 70 | 48.801 | 114.146 | 76.411 | 1.00 | 27.28 | C |
| ATOM | 2114 | C | PHE | C | 70 | 42.863 | 116.071 | 74.709 | 1.00 | 28.85 | C |
| ATOM | 2115 | O | PHE | C | 70 | 42.588 | 116.461 | 73.582 | 1.00 | 26.99 | C |
| ATOM | 2116 | N | LEU | C | 71 | 42.118 | 116.318 | 75.782 | 1.00 | 28.58 | C |
| ATOM | 2117 | CA | LEU | C | 71 | 40.959 | 117.162 | 75.732 | 1.00 | 28.71 | C |
| ATOM | 2118 | CB | LEU | C | 71 | 39.731 | 116.395 | 76.162 | 1.00 | 28.45 | C |
| ATOM | 2119 | CG | LEU | C | 71 | 39.367 | 115.092 | 75.456 | 1.00 | 29.25 | C |
| ATOM | 2120 | CD1 | LEU | C | 71 | 38.046 | 114.603 | 76.056 | 1.00 | 27.89 | C |
| ATOM | 2121 | CD2 | LEU | C | 71 | 39.225 | 115.301 | 73.938 | 1.00 | 27.22 | C |
| ATOM | 2122 | C | LEU | C | 71 | 41.272 | 118.232 | 76.807 | 1.00 | 30.35 | C |
| ATOM | 2123 | O | LEU | C | 71 | 42.115 | 118.021 | 77.681 | 1.00 | 30.67 | C |
| ATOM | 2124 | N | CYS | C | 72 | 40.627 | 119.391 | 76.748 | 1.00 | 31.16 | C |
| ATOM | 2125 | CA | CYS | C | 72 | 40.843 | 120.360 | 77.803 | 1.00 | 31.13 | C |
| ATOM | 2126 | CB | CYS | C | 72 | 42.019 | 121.276 | 77.429 | 1.00 | 29.58 | C |
| ATOM | 2127 | SG | CYS | C | 72 | 41.622 | 122.461 | 76.187 | 1.00 | 30.68 | C |
| ATOM | 2128 | C | CYS | C | 72 | 39.504 | 121.113 | 78.038 | 1.00 | 31.15 | C |
| ATOM | 2129 | O | CYS | C | 72 | 38.597 | 121.030 | 77.235 | 1.00 | 29.67 | C |
| ATOM | 2130 | N | SER | C | 73 | 39.397 | 121.801 | 79.163 | 1.00 | 32.66 | C |
| ATOM | 2131 | CA | SER | C | 73 | 38.186 | 122.551 | 79.535 | 1.00 | 33.86 | C |
| ATOM | 2132 | CB | SER | C | 73 | 38.349 | 123.261 | 80.901 | 1.00 | 34.85 | C |
| ATOM | 2133 | OG | SER | C | 73 | 38.996 | 122.441 | 81.888 | 1.00 | 38.83 | C |
| ATOM | 2134 | C | SER | C | 73 | 37.967 | 123.634 | 78.542 | 1.00 | 33.58 | C |
| ATOM | 2135 | O | SER | C | 73 | 38.910 | 124.109 | 77.910 | 1.00 | 33.11 | C |
| ATOM | 2136 | N | VAL | C | 74 | 36.728 | 124.059 | 78.463 | 1.00 | 33.20 | C |
| ATOM | 2137 | CA | VAL | C | 74 | 36.343 | 125.136 | 77.574 | 1.00 | 34.27 | C |
| ATOM | 2138 | CB | VAL | C | 74 | 34.828 | 125.039 | 77.341 | 1.00 | 37.14 | C |
| ATOM | 2139 | CG1 | VAL | C | 74 | 34.293 | 126.251 | 76.628 | 1.00 | 37.21 | C |
| ATOM | 2140 | CG2 | VAL | C | 74 | 34.556 | 123.782 | 76.538 | 1.00 | 39.64 | C |
| ATOM | 2141 | C | VAL | C | 74 | 36.687 | 126.443 | 78.291 | 1.00 | 32.50 | C |
| ATOM | 2142 | O | VAL | C | 74 | 36.436 | 126.593 | 79.460 | 1.00 | 34.41 | C |
| ATOM | 2143 | N | LEU | C | 75 | 37.247 | 127.398 | 77.597 | 1.00 | 30.48 | C |
| ATOM | 2144 | CA | LEU | C | 75 | 37.622 | 128.656 | 78.210 | 1.00 | 28.97 | C |
| ATOM | 2145 | CB | LEU | C | 75 | 39.036 | 129.036 | 77.774 | 1.00 | 26.73 | C |
| ATOM | 2146 | CG | LEU | C | 75 | 40.160 | 128.283 | 78.450 | 1.00 | 27.30 | C |
| ATOM | 2147 | CD1 | LEU | C | 75 | 41.464 | 128.495 | 77.697 | 1.00 | 22.74 | C |
| ATOM | 2148 | CD2 | LEU | C | 75 | 40.224 | 128.711 | 79.945 | 1.00 | 24.85 | C |
| ATOM | 2149 | C | LEU | C | 75 | 36.695 | 129.769 | 77.735 | 1.00 | 29.29 | C |
| ATOM | 2150 | O | LEU | C | 75 | 36.094 | 129.692 | 76.699 | 1.00 | 27.59 | C |
| ATOM | 2151 | N | PRO | C | 76 | 36.605 | 130.847 | 78.495 | 1.00 | 30.69 | C |
| ATOM | 2152 | CD | PRO | C | 76 | 37.366 | 131.186 | 79.698 | 1.00 | 31.44 | C |
| ATOM | 2153 | CA | PRO | C | 76 | 35.743 | 131.950 | 78.084 | 1.00 | 31.44 | C |
| ATOM | 2154 | CB | PRO | C | 76 | 35.901 | 132.978 | 79.196 | 1.00 | 30.76 | C |
| ATOM | 2155 | CG | PRO | C | 76 | 36.463 | 132.232 | 80.317 | 1.00 | 32.73 | C |
| ATOM | 2156 | C | PRO | C | 76 | 36.355 | 132.489 | 76.825 | 1.00 | 32.85 | C |
| ATOM | 2157 | O | PRO | C | 76 | 37.575 | 132.447 | 76.649 | 1.00 | 34.16 | C |
| ATOM | 2158 | N | THR | C | 77 | 35.535 | 133.051 | 75.958 | 1.00 | 33.52 | C |
| ATOM | 2159 | CA | THR | C | 77 | 36.066 | 133.594 | 74.703 | 1.00 | 33.22 | C |
| ATOM | 2160 | CB | THR | C | 77 | 34.901 | 134.010 | 73.762 | 1.00 | 33.73 | C |
| ATOM | 2161 | OG1 | THR | C | 77 | 34.470 | 132.842 | 73.063 | 1.00 | 35.39 | C |
| ATOM | 2162 | CG2 | THR | C | 77 | 35.341 | 135.011 | 72.758 | 1.00 | 34.82 | C |
| ATOM | 2163 | C | THR | C | 77 | 36.961 | 134.781 | 74.903 | 1.00 | 31.52 | C |
| ATOM | 2164 | O | THR | C | 77 | 37.970 | 134.918 | 74.232 | 1.00 | 31.18 | C |
| ATOM | 2165 | N | HIS | C | 78 | 36.523 | 135.641 | 75.814 | 1.00 | 30.38 | C |
| ATOM | 2166 | CA | HIS | C | 78 | 37.175 | 136.878 | 76.191 | 1.00 | 30.95 | C |
| ATOM | 2167 | CB | HIS | C | 78 | 36.335 | 137.987 | 75.633 | 1.00 | 30.49 | C |
| ATOM | 2168 | CG | HIS | C | 78 | 36.799 | 139.370 | 75.975 | 1.00 | 31.82 | C |
| ATOM | 2169 | CD2 | HIS | C | 78 | 38.023 | 139.858 | 76.274 | 1.00 | 32.73 | C |
| ATOM | 2170 | ND1 | HIS | C | 78 | 35.963 | 140.468 | 75.921 | 1.00 | 31.94 | C |
| ATOM | 2171 | CE1 | HIS | C | 78 | 36.647 | 141.568 | 76.166 | 1.00 | 30.16 | C |
| ATOM | 2172 | NE2 | HIS | C | 78 | 37.901 | 141.228 | 76.382 | 1.00 | 31.98 | C |
| ATOM | 2173 | C | HIS | C | 78 | 37.206 | 136.948 | 77.734 | 1.00 | 32.14 | C |
| ATOM | 2174 | O | HIS | C | 78 | 36.179 | 136.717 | 78.396 | 1.00 | 34.02 | C |
| ATOM | 2175 | N | TRP | C | 79 | 38.376 | 137.223 | 78.317 | 1.00 | 31.26 | C |
| ATOM | 2176 | CA | TRP | C | 79 | 38.498 | 137.303 | 79.793 | 1.00 | 29.12 | C |
| ATOM | 2177 | CB | TRP | C | 79 | 39.030 | 136.031 | 80.410 | 1.00 | 27.36 | C |
| ATOM | 2178 | CG | TRP | C | 79 | 38.637 | 135.893 | 81.845 | 1.00 | 26.00 | C |
| ATOM | 2179 | CD2 | TRP | C | 79 | 37.309 | 135.779 | 82.350 | 1.00 | 24.61 | C |
| ATOM | 2180 | CE2 | TRP | C | 79 | 37.412 | 135.610 | 83.759 | 1.00 | 23.81 | C |
| ATOM | 2181 | CE3 | TRP | C | 79 | 36.025 | 135.800 | 81.752 | 1.00 | 24.26 | C |
| ATOM | 2182 | CD1 | TRP | C | 79 | 39.474 | 135.796 | 82.940 | 1.00 | 25.05 | C |
| ATOM | 2183 | NE1 | TRP | C | 79 | 38.746 | 135.624 | 84.082 | 1.00 | 23.89 | C |
| ATOM | 2184 | CZ2 | TRP | C | 79 | 36.281 | 135.464 | 84.585 | 1.00 | 22.20 | C |
| ATOM | 2185 | CZ3 | TRP | C | 79 | 34.877 | 135.637 | 82.589 | 1.00 | 21.91 | C |
| ATOM | 2186 | CH2 | TRP | C | 79 | 35.029 | 135.476 | 83.985 | 1.00 | 22.26 | C |
| ATOM | 2187 | C | TRP | C | 79 | 39.448 | 138.393 | 80.182 | 1.00 | 29.42 | C |
| ATOM | 2188 | O | TRP | C | 79 | 40.271 | 138.853 | 79.365 | 1.00 | 29.08 | C |
| ATOM | 2189 | N | ARG | C | 80 | 39.338 | 138.791 | 81.446 | 1.00 | 29.85 | C |
| ATOM | 2190 | CA | ARG | C | 80 | 40.150 | 139.858 | 81.968 | 1.00 | 30.72 | C |
| ATOM | 2191 | CB | ARG | C | 80 | 39.502 | 140.476 | 83.178 | 1.00 | 30.49 | C |
| ATOM | 2192 | CG | ARG | C | 80 | 40.113 | 141.813 | 83.529 | 1.00 | 28.37 | C |
| ATOM | 2193 | CD | ARG | C | 80 | 39.276 | 142.500 | 84.545 | 1.00 | 28.39 | C |
| ATOM | 2194 | NE | ARG | C | 80 | 39.760 | 143.858 | 84.782 | 1.00 | 30.36 | C |
| ATOM | 2195 | CZ | ARG | C | 80 | 39.186 | 144.712 | 85.642 | 1.00 | 31.15 | C |
| ATOM | 2196 | NH1 | ARG | C | 80 | 38.102 | 144.341 | 86.338 | 1.00 | 29.10 | C |
| ATOM | 2197 | NH2 | ARG | C | 80 | 39.696 | 145.933 | 85.804 | 1.00 | 30.96 | C |
| ATOM | 2198 | C | ARG | C | 80 | 41.532 | 139.407 | 82.337 | 1.00 | 32.58 | C |
| ATOM | 2199 | O | ARG | C | 80 | 41.732 | 138.347 | 82.902 | 1.00 | 33.57 | C |
| ATOM | 2200 | N | CYS | C | 81 | 42.499 | 140.229 | 82.034 | 1.00 | 33.53 | C |
| ATOM | 2201 | CA | CYS | C | 81 | 43.807 | 139.827 | 82.354 | 1.00 | 37.57 | C |
| ATOM | 2202 | CB | CYS | C | 81 | 44.772 | 140.791 | 81.626 | 1.00 | 42.01 | C |
| ATOM | 2203 | SG | CYS | C | 81 | 45.438 | 142.042 | 82.691 | 1.00 | 53.97 | C |
| ATOM | 2204 | C | CYS | C | 81 | 44.001 | 139.739 | 83.892 | 1.00 | 36.01 | C |
| ATOM | 2205 | O | CYS | C | 81 | 43.448 | 140.497 | 84.659 | 1.00 | 37.08 | C |
| ATOM | 2206 | N | ASN | C | 82 | 44.771 | 138.761 | 84.320 | 1.00 | 35.22 | C |
| ATOM | 2207 | CA | ASN | C | 82 | 45.073 | 138.506 | 85.721 | 1.00 | 33.74 | C |
| ATOM | 2208 | CB | ASN | C | 82 | 45.813 | 139.655 | 86.375 | 1.00 | 34.09 | C |
| ATOM | 2209 | CG | ASN | C | 82 | 46.294 | 139.289 | 87.794 | 1.00 | 38.45 | C |
| ATOM | 2210 | OD1 | ASN | C | 82 | 46.852 | 138.191 | 88.022 | 1.00 | 40.27 | C |
| ATOM | 2211 | ND2 | ASN | C | 82 | 46.070 | 140.187 | 88.755 | 1.00 | 37.38 | C |
| ATOM | 2212 | C | ASN | C | 82 | 43.900 | 138.153 | 86.583 | 1.00 | 33.34 | C |
| ATOM | 2213 | O | ASN | C | 82 | 43.994 | 138.106 | 87.816 | 1.00 | 34.87 | C |
| ATOM | 2214 | N | LYS | C | 83 | 42.783 | 137.876 | 85.955 | 1.00 | 32.05 | C |
| ATOM | 2215 | CA | LYS | C | 83 | 41.608 | 137.522 | 86.712 | 1.00 | 31.58 | C |
| ATOM | 2216 | CB | LYS | C | 83 | 40.440 | 138.288 | 86.145 | 1.00 | 29.55 | C |
| ATOM | 2217 | CG | LYS | C | 83 | 39.139 | 137.866 | 86.680 | 1.00 | 29.22 | C |
| ATOM | 2218 | CD | LYS | C | 83 | 38.033 | 138.706 | 86.054 | 1.00 | 31.16 | C |
| ATOM | 2219 | CE | LYS | C | 83 | 36.701 | 138.362 | 86.696 | 1.00 | 32.13 | C |
| ATOM | 2220 | NZ | LYS | C | 83 | 35.706 | 139.373 | 86.340 | 1.00 | 34.84 | C |
| ATOM | 2221 | C | LYS | C | 83 | 41.301 | 135.996 | 86.743 | 1.00 | 32.81 | C |
| ATOM | 2222 | O | LYS | C | 83 | 41.455 | 135.264 | 85.745 | 1.00 | 33.87 | C |
| ATOM | 2223 | N | THR | C | 84 | 40.860 | 135.537 | 87.897 | 1.00 | 31.11 | C |
| ATOM | 2224 | CA | THR | C | 84 | 40.523 | 134.158 | 88.062 | 1.00 | 31.49 | C |
| ATOM | 2225 | CB | THR | C | 84 | 39.922 | 133.917 | 89.480 | 1.00 | 30.62 | C |
| ATOM | 2226 | OG1 | THR | C | 84 | 40.954 | 134.011 | 90.454 | 1.00 | 33.13 | C |
| ATOM | 2227 | CG2 | THR | C | 84 | 39.338 | 132.573 | 89.588 | 1.00 | 29.85 | C |
| ATOM | 2228 | C | THR | C | 84 | 39.493 | 133.773 | 86.988 | 1.00 | 31.84 | C |
| ATOM | 2229 | O | THR | C | 84 | 38.549 | 134.525 | 86.729 | 1.00 | 31.15 | C |
| ATOM | 2230 | N | LEU | C | 85 | 39.687 | 132.595 | 86.386 | 1.00 | 31.37 | C |
| ATOM | 2231 | CA | LEU | C | 85 | 38.795 | 132.089 | 85.376 | 1.00 | 31.62 | C |
| ATOM | 2232 | CB | LEU | C | 85 | 39.429 | 130.913 | 84.684 | 1.00 | 29.13 | C |
| ATOM | 2233 | CG | LEU | C | 85 | 40.698 | 131.259 | 83.940 | 1.00 | 28.22 | C |
| ATOM | 2234 | CD1 | LEU | C | 85 | 41.473 | 129.939 | 83.572 | 1.00 | 24.78 | C |
| ATOM | 2235 | CD2 | LEU | C | 85 | 40.280 | 132.111 | 82.757 | 1.00 | 23.91 | C |
| ATOM | 2236 | C | LEU | C | 85 | 37.493 | 131.622 | 85.967 | 1.00 | 32.88 | C |
| ATOM | 2237 | O | LEU | C | 85 | 37.441 | 131.243 | 87.128 | 1.00 | 33.69 | C |
| ATOM | 2238 | N | PRO | C | 86 | 36.419 | 131.620 | 85.165 | 1.00 | 33.95 | C |
| ATOM | 2239 | CD | PRO | C | 86 | 36.367 | 132.118 | 83.779 | 1.00 | 33.87 | C |
| ATOM | 2240 | CA | PRO | C | 86 | 35.099 | 131.163 | 85.650 | 1.00 | 35.26 | C |
| ATOM | 2241 | CB | PRO | C | 86 | 34.144 | 131.564 | 84.531 | 1.00 | 35.54 | C |
| ATOM | 2242 | CG | PRO | C | 86 | 35.054 | 131.590 | 83.296 | 1.00 | 35.69 | C |
| ATOM | 2243 | C | PRO | C | 86 | 35.031 | 129.649 | 85.985 | 1.00 | 36.54 | C |
| ATOM | 2244 | O | PRO | C | 86 | 34.149 | 129.228 | 86.731 | 1.00 | 37.79 | C |
| ATOM | 2245 | N | ILE | C | 87 | 35.934 | 128.838 | 85.440 | 1.00 | 35.53 | C |
| ATOM | 2246 | CA | ILE | C | 87 | 35.976 | 127.429 | 85.794 | 1.00 | 35.67 | C |
| ATOM | 2247 | CB | ILE | C | 87 | 35.491 | 126.532 | 84.687 | 1.00 | 37.21 | C |
| ATOM | 2248 | CG2 | ILE | C | 87 | 34.055 | 126.864 | 84.406 | 1.00 | 38.13 | C |
| ATOM | 2249 | CG1 | ILE | C | 87 | 36.294 | 126.759 | 83.384 | 1.00 | 38.60 | C |
| ATOM | 2250 | CD1 | ILE | C | 87 | 37.792 | 126.480 | 83.403 | 1.00 | 39.08 | C |
| ATOM | 2251 | C | ILE | C | 87 | 37.419 | 127.104 | 86.011 | 1.00 | 35.70 | C |
| ATOM | 2252 | O | ILE | C | 87 | 38.261 | 127.910 | 85.664 | 1.00 | 36.91 | C |
| ATOM | 2253 | N | ALA | C | 88 | 37.723 | 125.933 | 86.571 | 1.00 | 35.30 | C |
| ATOM | 2254 | CA | ALA | C | 88 | 39.109 | 125.555 | 86.770 | 1.00 | 33.45 | C |
| ATOM | 2255 | CB | ALA | C | 88 | 39.254 | 124.612 | 87.966 | 1.00 | 31.61 | C |
| ATOM | 2256 | C | ALA | C | 88 | 39.568 | 124.886 | 85.497 | 1.00 | 32.46 | C |
| ATOM | 2257 | O | ALA | C | 88 | 38.977 | 123.920 | 85.060 | 1.00 | 34.21 | C |
| ATOM | 2258 | N | PHE | C | 89 | 40.611 | 125.412 | 84.883 | 1.00 | 31.90 | C |
| ATOM | 2259 | CA | PHE | C | 89 | 41.134 | 124.821 | 83.665 | 1.00 | 31.21 | C |
| ATOM | 2260 | CB | PHE | C | 89 | 42.289 | 125.642 | 83.114 | 1.00 | 27.99 | C |
| ATOM | 2261 | CG | PHE | C | 89 | 42.709 | 125.213 | 81.755 | 1.00 | 27.26 | C |
| ATOM | 2262 | CD1 | PHE | C | 89 | 41.872 | 125.445 | 80.660 | 1.00 | 29.12 | C |
| ATOM | 2263 | CD2 | PHE | C | 89 | 43.901 | 124.555 | 81.545 | 1.00 | 25.87 | C |
| ATOM | 2264 | CE1 | PHE | C | 89 | 42.234 | 125.019 | 79.334 | 1.00 | 28.27 | C |
| ATOM | 2265 | CE2 | PHE | C | 89 | 44.273 | 124.127 | 80.269 | 1.00 | 26.58 | C |
| ATOM | 2266 | CZ | PHE | C | 89 | 43.448 | 124.353 | 79.141 | 1.00 | 27.43 | C |
| ATOM | 2267 | C | PHE | C | 89 | 41.634 | 123.392 | 83.929 | 1.00 | 32.04 | C |
| ATOM | 2268 | O | PHE | C | 89 | 42.427 | 123.166 | 84.862 | 1.00 | 33.97 | C |
| ATOM | 2269 | N | LYS | C | 90 | 41.155 | 122.443 | 83.127 | 1.00 | 31.27 | C |
| ATOM | 2270 | CA | LYS | C | 90 | 41.554 | 121.041 | 83.217 | 1.00 | 32.10 | C |
| ATOM | 2271 | CB | LYS | C | 90 | 40.364 | 120.142 | 83.557 | 1.00 | 33.56 | C |
| ATOM | 2272 | CG | LYS | C | 90 | 39.880 | 120.160 | 84.954 | 1.00 | 35.55 | C |
| ATOM | 2273 | CD | LYS | C | 90 | 38.577 | 119.425 | 84.962 | 1.00 | 37.60 | C |
| ATOM | 2274 | CE | LYS | C | 90 | 37.578 | 120.076 | 85.964 | 1.00 | 39.41 | C |
| ATOM | 2275 | NZ | LYS | C | 90 | 37.980 | 119.749 | 87.366 | 1.00 | 41.66 | C |
| ATOM | 2276 | C | LYS | C | 90 | 42.090 | 120.485 | 81.917 | 1.00 | 31.34 | C |
| ATOM | 2277 | O | LYS | C | 90 | 41.625 | 120.859 | 80.833 | 1.00 | 31.59 | C |
| ATOM | 2278 | N | VAL | C | 91 | 43.032 | 119.555 | 82.033 | 1.00 | 29.98 | C |
| ATOM | 2279 | CA | VAL | C | 91 | 43.589 | 118.859 | 80.887 | 1.00 | 28.72 | C |
| ATOM | 2280 | CB | VAL | C | 91 | 45.114 | 119.056 | 80.746 | 1.00 | 27.09 | C |
| ATOM | 2281 | CG1 | VAL | C | 91 | 45.644 | 118.106 | 79.660 | 1.00 | 25.99 | C |
| ATOM | 2282 | CG2 | VAL | C | 91 | 45.404 | 120.476 | 80.344 | 1.00 | 27.09 | C |
| ATOM | 2283 | C | VAL | C | 91 | 43.283 | 117.363 | 81.108 | 1.00 | 28.64 | C |
| ATOM | 2284 | O | VAL | C | 91 | 43.682 | 116.765 | 82.114 | 1.00 | 28.10 | C |
| ATOM | 2285 | N | VAL | C | 92 | 42.544 | 116.773 | 80.182 | 1.00 | 27.50 | C |
| ATOM | 2286 | CA | VAL | C | 92 | 42.219 | 115.365 | 80.287 | 1.00 | 28.23 | C |
| ATOM | 2287 | CB | VAL | C | 92 | 40.707 | 115.145 | 80.124 | 1.00 | 27.71 | C |
| ATOM | 2288 | CG1 | VAL | C | 92 | 40.374 | 113.659 | 80.240 | 1.00 | 25.28 | C |
| ATOM | 2289 | CG2 | VAL | C | 92 | 39.963 | 115.953 | 81.198 | 1.00 | 26.45 | C |
| ATOM | 2290 | C | VAL | C | 92 | 42.988 | 114.514 | 79.264 | 1.00 | 29.31 | C |
| ATOM | 2291 | O | VAL | C | 92 | 42.987 | 114.766 | 78.068 | 1.00 | 29.63 | C |
| ATOM | 2292 | N | ALA | C | 93 | 43.689 | 113.510 | 79.751 | 1.00 | 30.80 | C |
| ATOM | 2293 | CA | ALA | C | 93 | 44.472 | 112.622 | 78.885 | 1.00 | 30.83 | C |
| ATOM | 2294 | CB | ALA | C | 93 | 45.725 | 112.176 | 79.616 | 1.00 | 30.47 | C |
| ATOM | 2295 | C | ALA | C | 93 | 43.671 | 111.393 | 78.448 | 1.00 | 29.88 | C |
| ATOM | 2296 | O | ALA | C | 93 | 43.077 | 110.719 | 79.260 | 1.00 | 28.59 | C |
| ATOM | 2297 | N | LEU | C | 94 | 43.646 | 111.124 | 77.148 | 1.00 | 30.60 | C |
| ATOM | 2298 | CA | LEU | C | 94 | 42.886 | 109.976 | 76.655 | 1.00 | 31.86 | C |
| ATOM | 2299 | CB | LEU | C | 94 | 42.580 | 110.132 | 75.179 | 1.00 | 29.64 | C |
| ATOM | 2300 | CG | LEU | C | 94 | 41.646 | 111.326 | 74.957 | 1.00 | 31.24 | C |
| ATOM | 2301 | CD1 | LEU | C | 94 | 41.362 | 111.473 | 73.434 | 1.00 | 29.49 | C |
| ATOM | 2302 | CD2 | LEU | C | 94 | 40.340 | 111.161 | 75.764 | 1.00 | 28.40 | C |
| ATOM | 2303 | C | LEU | C | 94 | 43.744 | 108.737 | 76.937 | 1.00 | 32.35 | C |
| ATOM | 2304 | O | LEU | C | 94 | 43.432 | 107.922 | 77.843 | 1.00 | 32.28 | C |
| ATOM | 2305 | N | GLY | C | 95 | 44.862 | 108.645 | 76.230 | 1.00 | 30.49 | C |
| ATOM | 2306 | CA | GLY | C | 95 | 45.752 | 107.530 | 76.429 | 1.00 | 30.05 | C |
| ATOM | 2307 | C | GLY | C | 95 | 46.384 | 107.591 | 77.787 | 1.00 | 32.09 | C |
| ATOM | 2308 | O | GLY | C | 95 | 46.412 | 108.623 | 78.413 | 1.00 | 33.00 | C |
| ATOM | 2309 | N | ASP | C | 96 | 46.951 | 106.481 | 78.233 | 1.00 | 33.70 | C |
| ATOM | 2310 | CA | ASP | C | 96 | 47.562 | 106.365 | 79.550 | 1.00 | 34.61 | C |
| ATOM | 2311 | CB | ASP | C | 96 | 48.203 | 105.000 | 79.706 | 1.00 | 36.92 | C |
| ATOM | 2312 | CG | ASP | C | 96 | 47.201 | 103.887 | 79.732 | 1.00 | 39.47 | C |
| ATOM | 2313 | OD1 | ASP | C | 96 | 46.086 | 104.029 | 79.214 | 1.00 | 42.62 | C |
| ATOM | 2314 | OD2 | ASP | C | 96 | 47.536 | 102.830 | 80.263 | 1.00 | 41.77 | C |
| ATOM | 2315 | C | ASP | C | 96 | 48.617 | 107.385 | 79.817 | 1.00 | 35.22 | C |
| ATOM | 2316 | O | ASP | C | 96 | 49.458 | 107.645 | 78.901 | 1.00 | 36.65 | C |
| ATOM | 2317 | N | VAL | C | 97 | 48.575 | 107.942 | 81.052 | 1.00 | 33.06 | C |
| ATOM | 2318 | CA | VAL | C | 97 | 49.533 | 108.930 | 81.549 | 1.00 | 32.14 | C |
| ATOM | 2319 | CB | VAL | C | 97 | 49.042 | 110.391 | 81.307 | 1.00 | 31.23 | C |
| ATOM | 2320 | CG1 | VAL | C | 97 | 50.025 | 111.407 | 81.945 | 1.00 | 29.63 | C |
| ATOM | 2321 | CG2 | VAL | C | 97 | 48.952 | 110.644 | 79.814 | 1.00 | 26.08 | C |
| ATOM | 2322 | C | VAL | C | 97 | 49.745 | 108.686 | 83.036 | 1.00 | 33.72 | C |
| ATOM | 2323 | O | VAL | C | 97 | 48.774 | 106.638 | 83.785 | 1.00 | 35.51 | C |
| ATOM | 2324 | N | PRO | C | 98 | 51.012 | 108.519 | 83.486 | 1.00 | 33.95 | C |
| ATOM | 2325 | CD | PRO | C | 98 | 52.221 | 108.481 | 82.644 | 1.00 | 33.10 | C |
| ATOM | 2326 | CA | PRO | C | 98 | 51.346 | 108.257 | 84.914 | 1.00 | 35.09 | C |
| ATOM | 2327 | CB | PRO | C | 98 | 52.857 | 108.015 | 84.906 | 1.00 | 32.98 | C |
| ATOM | 2328 | CG | PRO | C | 98 | 53.172 | 107.656 | 83.509 | 1.00 | 33.49 | C |
| ATOM | 2329 | C | PRO | C | 98 | 50.985 | 109.362 | 85.910 | 1.00 | 36.46 | C |
| ATOM | 2330 | O | PRO | C | 98 | 51.454 | 110.498 | 85.770 | 1.00 | 37.84 | C |
| ATOM | 2331 | N | ASP | C | 99 | 50.147 | 109.039 | 86.900 | 1.00 | 36.78 | C |
| ATOM | 2332 | CA | ASP | C | 99 | 49.774 | 110.020 | 87.913 | 1.00 | 36.76 | C |
| ATOM | 2333 | CB | ASP | C | 99 | 49.275 | 109.351 | 89.155 | 1.00 | 36.18 | C |
| ATOM | 2334 | CG | ASP | C | 99 | 47.854 | 108.942 | 89.040 | 1.00 | 38.61 | C |
| ATOM | 2335 | OD1 | ASP | C | 99 | 47.262 | 109.119 | 87.969 | 1.00 | 37.95 | C |
| ATOM | 2336 | OD2 | ASP | C | 99 | 47.300 | 108.440 | 90.028 | 1.00 | 41.34 | C |
| ATOM | 2337 | C | ASP | C | 99 | 51.044 | 110.736 | 88.260 | 1.00 | 38.07 | C |
| ATOM | 2338 | O | ASP | C | 99 | 52.111 | 110.127 | 88.280 | 1.00 | 39.22 | C |
| ATOM | 2339 | N | GLY | C | 100 | 50.962 | 112.042 | 88.488 | 1.00 | 38.69 | C |
| ATOM | 2340 | CA | GLY | C | 100 | 52.170 | 112.788 | 88.831 | 1.00 | 37.67 | C |
| ATOM | 2341 | C | GLY | C | 100 | 52.920 | 113.373 | 87.636 | 1.00 | 36.97 | C |
| ATOM | 2342 | O | GLY | C | 100 | 53.934 | 114.048 | 87.809 | 1.00 | 36.77 | C |
| ATOM | 2343 | N | THR | C | 101 | 52.459 | 113.104 | 86.417 | 1.00 | 34.74 | C |
| ATOM | 2344 | CA | THR | C | 101 | 53.129 | 113.715 | 85.299 | 1.00 | 32.15 | C |
| ATOM | 2345 | CB | THR | C | 101 | 52.636 | 113.124 | 83.987 | 1.00 | 30.65 | C |
| ATOM | 2346 | OG1 | THR | C | 101 | 52.973 | 111.718 | 83.947 | 1.00 | 30.02 | C |
| ATOM | 2347 | CG2 | THR | C | 101 | 53.269 | 113.847 | 82.824 | 1.00 | 27.13 | C |
| ATOM | 2348 | C | THR | C | 101 | 52.831 | 115.217 | 85.401 | 1.00 | 32.42 | C |
| ATOM | 2349 | O | THR | C | 101 | 51.710 | 115.643 | 85.741 | 1.00 | 33.86 | C |
| ATOM | 2350 | N | LEU | C | 102 | 53.861 | 116.011 | 85.171 | 1.00 | 32.25 | C |
| ATOM | 2351 | CA | LEU | C | 102 | 53.751 | 117.454 | 85.186 | 1.00 | 32.03 | C |
| ATOM | 2352 | CB | LEU | C | 102 | 55.152 | 118.083 | 85.297 | 1.00 | 32.15 | C |
| ATOM | 2353 | CG | LEU | C | 102 | 55.333 | 119.619 | 85.219 | 1.00 | 32.70 | C |
| ATOM | 2354 | CD1 | LEU | C | 102 | 54.372 | 120.405 | 86.209 | 1.00 | 32.03 | C |
| ATOM | 2355 | CD2 | LEU | C | 102 | 56.762 | 119.915 | 85.501 | 1.00 | 29.31 | C |
| ATOM | 2356 | C | LEU | C | 102 | 53.089 | 117.888 | 83.881 | 1.00 | 31.54 | C |
| ATOM | 2357 | O | LEU | C | 102 | 53.370 | 117.298 | 82.847 | 1.00 | 30.94 | C |
| ATOM | 2358 | N | VAL | C | 103 | 52.201 | 118.892 | 83.937 | 1.00 | 30.83 | C |
| ATOM | 2359 | CA | VAL | C | 103 | 51.513 | 119.428 | 82.764 | 1.00 | 29.27 | C |
| ATOM | 2360 | CB | VAL | C | 103 | 50.008 | 119.036 | 82.754 | 1.00 | 27.05 | C |
| ATOM | 2361 | CG1 | VAL | C | 103 | 49.317 | 119.600 | 81.514 | 1.00 | 23.70 | C |
| ATOM | 2362 | CG2 | VAL | C | 103 | 49.885 | 117.534 | 82.819 | 1.00 | 27.14 | C |
| ATOM | 2363 | C | VAL | C | 103 | 51.596 | 120.936 | 82.928 | 1.00 | 29.84 | C |
| ATOM | 2364 | O | VAL | C | 103 | 51.375 | 121.469 | 84.028 | 1.00 | 30.98 | C |
| ATOM | 2365 | N | THR | C | 104 | 51.851 | 121.638 | 81.846 | 1.00 | 29.90 | C |
| ATOM | 2366 | CA | THR | C | 104 | 51.943 | 123.077 | 81.930 | 1.00 | 30.30 | C |
| ATOM | 2367 | CB | THR | C | 104 | 53.383 | 123.569 | 81.839 | 1.00 | 30.59 | C |
| ATOM | 2368 | OG1 | THR | C | 104 | 53.847 | 123.330 | 80.500 | 1.00 | 33.27 | C |
| ATOM | 2369 | CG2 | THR | C | 104 | 54.279 | 122.869 | 82.847 | 1.00 | 29.33 | C |
| ATOM | 2370 | C | THR | C | 104 | 51.232 | 123.726 | 80.757 | 1.00 | 31.36 | C |
| ATOM | 2371 | O | THR | C | 104 | 50.983 | 123.070 | 79.728 | 1.00 | 30.44 | C |
| ATOM | 2372 | N | VAL | C | 105 | 50.962 | 125.027 | 80.904 | 1.00 | 30.15 | C |
| ATOM | 2373 | CA | VAL | C | 105 | 50.297 | 125.794 | 79.858 | 1.00 | 29.66 | C |
| ATOM | 2374 | CB | VAL | C | 105 | 48.889 | 126.282 | 80.322 | 1.00 | 28.13 | C |
| ATOM | 2375 | CG1 | VAL | C | 105 | 48.298 | 127.227 | 79.341 | 1.00 | 25.68 | C |
| ATOM | 2376 | CG2 | VAL | C | 105 | 47.978 | 125.094 | 80.454 | 1.00 | 26.83 | C |
| ATOM | 2377 | C | VAL | C | 105 | 51.117 | 126.990 | 79.513 | 1.00 | 30.18 | C |
| ATOM | 2378 | O | VAL | C | 105 | 51.651 | 127.626 | 80.382 | 1.00 | 32.01 | C |
| ATOM | 2379 | N | MET | C | 106 | 51.216 | 127.301 | 78.236 | 1.00 | 32.58 | C |
| ATOM | 2380 | CA | MET | C | 106 | 51.950 | 128.485 | 77.762 | 1.00 | 33.02 | C |
| ATOM | 2381 | CB | MET | C | 106 | 53.284 | 128.114 | 77.163 | 1.00 | 32.80 | C |
| ATOM | 2382 | CG | MET | C | 106 | 54.231 | 127.386 | 78.078 | 1.00 | 34.70 | C |
| ATOM | 2383 | SD | MET | C | 106 | 55.899 | 127.243 | 77.261 | 1.00 | 41.58 | C |
| ATOM | 2384 | CE | MET | C | 106 | 55.936 | 125.550 | 76.777 | 1.00 | 38.71 | C |
| ATOM | 2385 | C | MET | C | 106 | 51.052 | 129.081 | 76.672 | 1.00 | 34.08 | C |
| ATOM | 2386 | O | MET | C | 106 | 50.234 | 128.378 | 76.050 | 1.00 | 33.39 | C |
| ATOM | 2387 | N | ALA | C | 107 | 51.188 | 130.374 | 76.434 | 1.00 | 34.43 | C |
| ATOM | 2388 | CA | ALA | C | 107 | 50.336 | 130.986 | 75.453 | 1.00 | 35.00 | C |
| ATOM | 2389 | CB | ALA | C | 107 | 49.152 | 131.663 | 76.142 | 1.00 | 33.40 | C |
| ATOM | 2390 | C | ALA | C | 107 | 51.118 | 131.988 | 74.650 | 1.00 | 35.96 | C |
| ATOM | 2391 | O | ALA | C | 107 | 52.165 | 132.447 | 75.096 | 1.00 | 36.63 | C |
| ATOM | 2392 | N | GLY | C | 108 | 50.603 | 132.325 | 73.467 | 1.00 | 35.39 | C |
| ATOM | 2393 | CA | GLY | C | 108 | 51.271 | 133.308 | 72.636 | 1.00 | 36.08 | C |
| ATOM | 2394 | C | GLY | C | 108 | 50.420 | 133.498 | 71.415 | 1.00 | 36.70 | C |
| ATOM | 2395 | O | GLY | C | 108 | 49.379 | 132.843 | 71.315 | 1.00 | 38.41 | C |
| ATOM | 2396 | N | ASN | C | 109 | 50.818 | 134.397 | 70.526 | 1.00 | 36.52 | C |
| ATOM | 2397 | CA | ASN | C | 109 | 50.102 | 134.596 | 69.294 | 1.00 | 37.94 | C |
| ATOM | 2398 | CB | ASN | C | 109 | 48.672 | 135.117 | 69.537 | 1.00 | 37.11 | C |
| ATOM | 2399 | CG | ASN | C | 109 | 48.638 | 136.510 | 69.962 | 1.00 | 36.95 | C |
| ATOM | 2400 | OD1 | ASN | C | 109 | 49.667 | 137.165 | 70.023 | 1.00 | 39.21 | C |
| ATOM | 2401 | ND2 | ASN | C | 109 | 47.451 | 137.005 | 70.263 | 1.00 | 36.94 | C |
| ATOM | 2402 | C | ASN | C | 109 | 50.929 | 135.545 | 68.463 | 1.00 | 39.42 | C |
| ATOM | 2403 | O | ASN | C | 109 | 52.010 | 135.936 | 68.916 | 1.00 | 37.70 | C |
| ATOM | 2404 | N | ASP | C | 110 | 50.437 | 135.931 | 67.279 | 1.00 | 40.87 | C |
| ATOM | 2405 | CA | ASP | C | 110 | 51.249 | 136.807 | 66.444 | 1.00 | 44.38 | C |
| ATOM | 2406 | CB | ASP | C | 110 | 50.757 | 136.831 | 65.002 | 1.00 | 45.50 | C |
| ATOM | 2407 | CG | ASP | C | 110 | 49.457 | 137.600 | 64.827 | 1.00 | 48.02 | C |
| ATOM | 2408 | OD1 | ASP | C | 110 | 49.000 | 137.710 | 63.665 | 1.00 | 50.16 | C |
| ATOM | 2409 | OD2 | ASP | C | 110 | 48.884 | 138.083 | 65.819 | 1.00 | 47.93 | C |
| ATOM | 2410 | C | ASP | C | 110 | 51.423 | 138.245 | 66.926 | 1.00 | 45.71 | C |
| ATOM | 2411 | O | ASP | C | 110 | 51.916 | 139.092 | 66.172 | 1.00 | 46.80 | C |
| ATOM | 2412 | N | GLU | C | 111 | 51.058 | 138.510 | 68.179 | 1.00 | 45.59 | C |
| ATOM | 2413 | CA | GLU | C | 111 | 51.189 | 139.849 | 68.713 | 1.00 | 46.51 | C |
| ATOM | 2414 | CB | GLU | C | 111 | 49.821 | 140.466 | 68.856 | 1.00 | 47.25 | C |
| ATOM | 2415 | CG | GLU | C | 111 | 49.899 | 141.892 | 69.362 | 1.00 | 51.67 | C |
| ATOM | 2416 | CD | GLU | C | 111 | 48.832 | 142.802 | 68.744 | 1.00 | 54.57 | C |
| ATOM | 2417 | OE1 | GLU | C | 111 | 48.471 | 143.776 | 69.436 | 1.00 | 55.28 | C |
| ATOM | 2418 | OE2 | GLU | C | 111 | 48.371 | 142.554 | 67.574 | 1.00 | 54.30 | C |
| ATOM | 2419 | C | GLU | C | 111 | 51.944 | 139.911 | 70.042 | 1.00 | 45.60 | C |
| ATOM | 2420 | O | GLU | C | 111 | 52.673 | 140.868 | 70.307 | 1.00 | 45.89 | C |
| ATOM | 2421 | N | ASN | C | 112 | 51.751 | 138.900 | 70.874 | 1.00 | 43.35 | C |
| ATOM | 2422 | CA | ASN | C | 112 | 52.440 | 138.799 | 72.147 | 1.00 | 42.37 | C |
| ATOM | 2423 | CB | ASN | C | 112 | 51.410 | 138.797 | 73.244 | 1.00 | 41.08 | C |
| ATOM | 2424 | CG | ASN | C | 112 | 52.010 | 138.762 | 74.599 | 1.00 | 39.23 | C |
| ATOM | 2425 | OD1 | ASN | C | 112 | 53.181 | 138.422 | 74.759 | 1.00 | 40.25 | C |
| ATOM | 2426 | ND2 | ASN | C | 112 | 51.195 | 139.070 | 75.617 | 1.00 | 36.81 | C |
| ATOM | 2427 | C | ASN | C | 112 | 53.042 | 137.423 | 71.928 | 1.00 | 42.67 | C |
| ATOM | 2428 | O | ASN | C | 112 | 52.326 | 136.426 | 71.969 | 1.00 | 44.25 | C |
| ATOM | 2429 | N | TYR | C | 113 | 54.341 | 137.351 | 71.676 | 1.00 | 41.86 | C |
| ATOM | 2430 | CA | TYR | C | 113 | 54.901 | 136.063 | 71.324 | 1.00 | 42.12 | C |
| ATOM | 2431 | CB | TYR | C | 113 | 56.260 | 136.270 | 70.666 | 1.00 | 42.12 | C |
| ATOM | 2432 | CG | TYR | C | 113 | 57.368 | 136.494 | 71.632 | 1.00 | 43.99 | C |
| ATOM | 2433 | CD1 | TYR | C | 113 | 58.047 | 135.414 | 72.185 | 1.00 | 45.33 | C |
| ATOM | 2434 | CE1 | TYR | C | 113 | 59.034 | 135.598 | 73.157 | 1.00 | 46.73 | C |
| ATOM | 2435 | CD2 | TYR | C | 113 | 57.704 | 137.776 | 72.058 | 1.00 | 44.61 | C |
| ATOM | 2436 | CE2 | TYR | C | 113 | 58.691 | 137.977 | 73.030 | 1.00 | 45.95 | C |
| ATOM | 2437 | CZ | TYR | C | 113 | 59.338 | 136.876 | 73.579 | 1.00 | 47.28 | C |
| ATOM | 2438 | OH | TYR | C | 113 | 60.212 | 137.008 | 74.631 | 1.00 | 49.42 | C |
| ATOM | 2439 | C | TYR | C | 113 | 54.941 | 135.069 | 72.475 | 1.00 | 41.75 | C |
| ATOM | 2440 | O | TYR | C | 113 | 55.018 | 133.847 | 72.276 | 1.00 | 41.13 | C |
| ATOM | 2441 | N | SER | C | 114 | 54.831 | 135.600 | 73.686 | 1.00 | 41.69 | C |
| ATOM | 2442 | CA | SER | C | 114 | 54.855 | 134.757 | 74.855 | 1.00 | 39.84 | C |
| ATOM | 2443 | CB | SER | C | 114 | 56.290 | 134.441 | 75.178 | 1.00 | 39.26 | C |
| ATOM | 2444 | OG | SER | C | 114 | 56.306 | 133.396 | 76.131 | 1.00 | 44.63 | C |
| ATOM | 2445 | C | SER | C | 114 | 54.137 | 135.424 | 76.047 | 1.00 | 39.49 | C |
| ATOM | 2446 | O | SER | C | 114 | 54.733 | 136.165 | 76.830 | 1.00 | 40.75 | C |
| ATOM | 2447 | N | ALA | C | 115 | 52.850 | 135.143 | 76.187 | 1.00 | 37.79 | C |
| ATOM | 2448 | CA | ALA | C | 115 | 52.057 | 135.711 | 77.251 | 1.00 | 36.23 | C |
| ATOM | 2449 | CB | ALA | C | 115 | 50.617 | 135.390 | 77.017 | 1.00 | 35.84 | C |
| ATOM | 2450 | C | ALA | C | 115 | 52.445 | 135.251 | 78.647 | 1.00 | 36.33 | C |
| ATOM | 2451 | O | ALA | C | 115 | 52.738 | 134.077 | 78.868 | 1.00 | 36.02 | C |
| ATOM | 2452 | N | GLU | C | 116 | 52.402 | 136.192 | 79.587 | 1.00 | 36.44 | C |
| ATOM | 2453 | CA | GLU | C | 116 | 52.701 | 135.939 | 80.972 | 1.00 | 36.45 | C |
| ATOM | 2454 | CB | GLU | C | 116 | 53.039 | 137.238 | 81.682 | 1.00 | 39.39 | C |
| ATOM | 2455 | CG | GLU | C | 116 | 52.741 | 137.200 | 83.178 | 1.00 | 45.98 | C |
| ATOM | 2456 | CD | GLU | C | 116 | 53.426 | 138.348 | 83.935 | 1.00 | 50.07 | C |
| ATOM | 2457 | OE1 | GLU | C | 116 | 52.920 | 139.520 | 83.940 | 1.00 | 51.52 | C |
| ATOM | 2458 | OE2 | GLU | C | 116 | 54.502 | 138.063 | 84.515 | 1.00 | 52.79 | C |
| ATOM | 2459 | C | GLU | C | 116 | 51.501 | 135.284 | 81.629 | 1.00 | 35.12 | C |
| ATOM | 2460 | O | GLU | C | 116 | 50.383 | 135.785 | 81.553 | 1.00 | 32.87 | C |
| ATOM | 2461 | N | LEU | C | 117 | 51.770 | 134.188 | 82.318 | 1.00 | 33.65 | C |
| ATOM | 2462 | CA | LEU | C | 117 | 50.730 | 133.436 | 82.966 | 1.00 | 33.68 | C |
| ATOM | 2463 | CB | LEU | C | 117 | 50.688 | 132.049 | 82.353 | 1.00 | 33.11 | C |
| ATOM | 2464 | CG | LEU | C | 117 | 50.501 | 131.960 | 80.874 | 1.00 | 31.83 | C |
| ATOM | 2465 | CD1 | LEU | C | 117 | 50.429 | 130.470 | 80.544 | 1.00 | 31.78 | C |
| ATOM | 2466 | CD2 | LEU | C | 117 | 49.226 | 132.707 | 80.476 | 1.00 | 31.58 | C |
| ATOM | 2467 | C | LEU | C | 117 | 50.960 | 133.270 | 84.449 | 1.00 | 34.36 | C |
| ATOM | 2468 | O | LEU | C | 117 | 52.006 | 133.603 | 84.941 | 1.00 | 36.24 | C |
| ATOM | 2469 | N | ARG | C | 118 | 49.986 | 132.726 | 85.161 | 1.00 | 34.54 | C |
| ATOM | 2470 | CA | ARG | C | 118 | 50.146 | 132.469 | 86.574 | 1.00 | 35.11 | C |
| ATOM | 2471 | CB | ARG | C | 118 | 49.507 | 133.568 | 87.391 | 1.00 | 36.75 | C |
| ATOM | 2472 | CG | ARG | C | 118 | 50.354 | 134.845 | 87.411 | 1.00 | 41.11 | C |
| ATOM | 2473 | CD | ARG | C | 118 | 49.644 | 135.919 | 88.225 | 1.00 | 44.78 | C |
| ATOM | 2474 | NE | ARG | C | 118 | 49.084 | 135.339 | 89.454 | 1.00 | 48.38 | C |
| ATOM | 2475 | CZ | ARG | C | 118 | 48.415 | 136.032 | 90.373 | 1.00 | 49.92 | C |
| ATOM | 2476 | NH1 | ARG | C | 118 | 48.238 | 137.346 | 90.190 | 1.00 | 49.36 | C |
| ATOM | 2477 | NH2 | ARG | C | 118 | 47.898 | 135.408 | 91.451 | 1.00 | 50.71 | C |
| ATOM | 2478 | C | ARG | C | 118 | 49.541 | 131.108 | 86.928 | 1.00 | 34.92 | C |
| ATOM | 2479 | O | ARG | C | 118 | 48.424 | 130.767 | 86.479 | 1.00 | 36.54 | C |
| ATOM | 2480 | N | ASN | C | 119 | 50.274 | 130.327 | 87.723 | 1.00 | 32.22 | C |
| ATOM | 2481 | CA | ASN | C | 119 | 49.833 | 129.008 | 88.108 | 1.00 | 30.03 | C |
| ATOM | 2482 | CB | ASN | C | 119 | 48.542 | 129.101 | 88.872 | 1.00 | 29.21 | C |
| ATOM | 2483 | CG | ASN | C | 119 | 48.569 | 130.238 | 89.843 | 1.00 | 29.80 | C |
| ATOM | 2484 | OD1 | ASN | C | 119 | 49.581 | 130.460 | 90.512 | 1.00 | 28.32 | C |
| ATOM | 2485 | ND2 | ASN | C | 119 | 47.464 | 130.960 | 89.947 | 1.00 | 27.96 | C |
| ATOM | 2486 | C | ASN | C | 119 | 49.595 | 128.161 | 86.890 | 1.00 | 29.45 | C |
| ATOM | 2487 | O | ASN | C | 119 | 48.564 | 127.496 | 86.844 | 1.00 | 30.33 | C |
| ATOM | 2488 | N | ALA | C | 120 | 50.506 | 128.202 | 85.914 | 1.00 | 27.06 | C |
| ATOM | 2489 | CA | ALA | C | 120 | 50.354 | 127.436 | 84.697 | 1.00 | 26.09 | C |
| ATOM | 2490 | CB | ALA | C | 120 | 51.064 | 128.126 | 83.526 | 1.00 | 24.59 | C |
| ATOM | 2491 | C | ALA | C | 120 | 50.911 | 126.055 | 84.865 | 1.00 | 26.24 | C |
| ATOM | 2492 | O | ALA | C | 120 | 51.397 | 125.453 | 83.916 | 1.00 | 28.50 | C |
| ATOM | 2493 | N | THR | C | 121 | 50.738 | 125.517 | 86.043 | 1.00 | 25.96 | C |
| ATOM | 2494 | CA | THR | C | 121 | 51.307 | 124.243 | 86.323 | 1.00 | 27.16 | C |
| ATOM | 2495 | CB | THR | C | 121 | 52.485 | 124.486 | 87.214 | 1.00 | 27.20 | C |
| ATOM | 2496 | OG1 | THR | C | 121 | 53.631 | 124.396 | 86.398 | 1.00 | 26.37 | C |
| ATOM | 2497 | CG2 | THR | C | 121 | 52.550 | 123.534 | 88.392 | 1.00 | 25.56 | C |
| ATOM | 2498 | C | THR | C | 121 | 50.338 | 123.339 | 86.997 | 1.00 | 28.42 | C |
| ATOM | 2499 | O | THR | C | 121 | 49.445 | 123.819 | 87.738 | 1.00 | 27.33 | C |
| ATOM | 2500 | N | ALA | C | 122 | 50.504 | 122.039 | 86.717 | 1.00 | 28.07 | C |
| ATOM | 2501 | CA | ALA | C | 122 | 49.666 | 121.019 | 87.335 | 1.00 | 28.28 | C |
| ATOM | 2502 | CB | ALA | C | 122 | 48.226 | 121.139 | 86.829 | 1.00 | 28.29 | C |
| ATOM | 2503 | C | ALA | C | 122 | 50.180 | 119.574 | 87.164 | 1.00 | 30.64 | C |
| ATOM | 2504 | O | ALA | C | 122 | 51.129 | 119.271 | 86.402 | 1.00 | 32.48 | C |
| ATOM | 2505 | N | ALA | C | 123 | 49.540 | 118.652 | 87.854 | 1.00 | 31.13 | C |
| ATOM | 2506 | CA | ALA | C | 123 | 49.983 | 117.275 | 87.799 | 1.00 | 31.10 | C |
| ATOM | 2507 | CB | ALA | C | 123 | 50.610 | 116.894 | 89.118 | 1.00 | 28.18 | C |
| ATOM | 2508 | C | ALA | C | 123 | 48.823 | 116.350 | 87.490 | 1.00 | 31.91 | C |
| ATOM | 2509 | O | ALA | C | 123 | 47.712 | 116.507 | 88.017 | 1.00 | 31.92 | C |
| ATOM | 2510 | N | MET | C | 124 | 49.104 | 115.357 | 86.655 | 1.00 | 32.45 | C |
| ATOM | 2511 | CA | MET | C | 124 | 48.090 | 114.400 | 86.271 | 1.00 | 31.80 | C |
| ATOM | 2512 | CB | MET | C | 124 | 48.653 | 113.502 | 85.192 | 1.00 | 32.66 | C |
| ATOM | 2513 | CG | MET | C | 124 | 48.446 | 113.932 | 83.784 | 1.00 | 33.31 | C |
| ATOM | 2514 | SD | MET | C | 124 | 46.739 | 113.887 | 83.308 | 1.00 | 32.34 | C |
| ATOM | 2515 | CE | MET | C | 124 | 46.912 | 114.593 | 81.700 | 1.00 | 34.85 | C |
| ATOM | 2516 | C | MET | C | 124 | 47.697 | 113.533 | 87.439 | 1.00 | 31.69 | C |
| ATOM | 2517 | O | MET | C | 124 | 48.568 | 113.066 | 88.175 | 1.00 | 32.88 | C |
| ATOM | 2518 | N | LYS | C | 125 | 46.398 | 113.280 | 87.574 | 1.00 | 31.81 | C |
| ATOM | 2519 | CA | LYS | C | 125 | 45.847 | 112.411 | 88.612 | 1.00 | 31.27 | C |
| ATOM | 2520 | CB | LYS | C | 125 | 45.397 | 113.225 | 89.817 | 1.00 | 31.69 | C |
| ATOM | 2521 | CG | LYS | C | 125 | 45.010 | 112.382 | 91.053 | 1.00 | 34.10 | C |
| ATOM | 2522 | CD | LYS | C | 125 | 45.866 | 111.098 | 91.109 | 1.00 | 37.83 | C |
| ATOM | 2523 | CE | LYS | C | 125 | 45.960 | 110.495 | 92.554 | 1.00 | 40.64 | C |
| ATOM | 2524 | NZ | LYS | C | 125 | 46.890 | 109.295 | 92.636 | 1.00 | 41.27 | C |
| ATOM | 2525 | C | LYS | C | 125 | 44.674 | 111.732 | 87.907 | 1.00 | 31.95 | C |
| ATOM | 2526 | O | LYS | C | 125 | 43.734 | 112.400 | 87.474 | 1.00 | 29.63 | C |
| ATOM | 2527 | N | ASN | C | 126 | 44.771 | 110.397 | 87.784 | 1.00 | 33.26 | C |
| ATOM | 2528 | CA | ASN | C | 126 | 43.789 | 109.540 | 87.075 | 1.00 | 32.57 | C |
| ATOM | 2529 | CB | ASN | C | 126 | 42.504 | 109.264 | 87.888 | 1.00 | 33.62 | C |
| ATOM | 2530 | CG | ASN | C | 126 | 42.782 | 108.847 | 89.353 | 1.00 | 38.09 | C |
| ATOM | 2531 | OD1 | ASN | C | 126 | 43.523 | 107.889 | 89.662 | 1.00 | 38.06 | C |
| ATOM | 2532 | ND2 | ASN | C | 126 | 42.173 | 109.594 | 90.272 | 1.00 | 40.50 | C |
| ATOM | 2533 | C | ASN | C | 126 | 43.403 | 110.182 | 85.746 | 1.00 | 32.07 | C |
| ATOM | 2534 | O | ASN | C | 126 | 42.228 | 110.396 | 85.474 | 1.00 | 32.46 | C |
| ATOM | 2535 | N | GLN | C | 127 | 44.393 | 110.532 | 84.929 | 1.00 | 30.75 | C |
| ATOM | 2536 | CA | GLN | C | 127 | 44.131 | 111.103 | 83.609 | 1.00 | 30.47 | C |
| ATOM | 2537 | CB | GLN | C | 127 | 43.140 | 110.213 | 82.862 | 1.00 | 29.34 | C |
| ATOM | 2538 | CG | GLN | C | 127 | 43.621 | 108.773 | 82.781 | 1.00 | 30.08 | C |
| ATOM | 2539 | CD | GLN | C | 127 | 44.761 | 108.612 | 81.804 | 1.00 | 31.43 | C |
| ATOM | 2540 | OE1 | GLN | C | 127 | 45.812 | 108.106 | 82.139 | 1.00 | 31.26 | C |
| ATOM | 2541 | NE2 | GLN | C | 127 | 44.551 | 109.056 | 80.578 | 1.00 | 32.83 | C |
| ATOM | 2542 | C | GLN | C | 127 | 43.648 | 112.568 | 83.546 | 1.00 | 30.84 | C |
| ATOM | 2543 | O | GLN | C | 127 | 43.379 | 113.080 | 82.462 | 1.00 | 30.85 | C |
| ATOM | 2544 | N | VAL | C | 128 | 43.572 | 113.248 | 84.684 | 1.00 | 29.27 | C |
| ATOM | 2545 | CA | VAL | C | 128 | 43.131 | 114.616 | 84.669 | 1.00 | 28.71 | C |
| ATOM | 2546 | CB | VAL | C | 128 | 41.757 | 114.722 | 85.350 | 1.00 | 28.20 | C |
| ATOM | 2547 | CG1 | VAL | C | 128 | 41.377 | 116.137 | 85.619 | 1.00 | 26.06 | C |
| ATOM | 2548 | CG2 | VAL | C | 128 | 40.741 | 114.059 | 84.481 | 1.00 | 29.82 | C |
| ATOM | 2549 | C | VAL | C | 128 | 44.100 | 115.519 | 85.387 | 1.00 | 29.43 | C |
| ATOM | 2550 | O | VAL | C | 128 | 44.414 | 115.317 | 86.525 | 1.00 | 29.88 | C |
| ATOM | 2551 | N | ALA | C | 129 | 44.566 | 116.543 | 84.726 | 1.00 | 29.90 | C |
| ATOM | 2552 | CA | ALA | C | 129 | 45.454 | 117.457 | 85.400 | 1.00 | 30.97 | C |
| ATOM | 2553 | CB | ALA | C | 129 | 46.670 | 117.828 | 84.475 | 1.00 | 30.54 | C |
| ATOM | 2554 | C | ALA | C | 129 | 44.572 | 118.674 | 85.659 | 1.00 | 30.83 | C |
| ATOM | 2555 | O | ALA | C | 129 | 44.106 | 119.301 | 84.705 | 1.00 | 31.50 | C |
| ATOM | 2556 | N | ARG | C | 130 | 44.328 | 119.020 | 86.921 | 1.00 | 31.92 | C |
| ATOM | 2557 | CA | ARG | C | 130 | 43.473 | 120.190 | 87.208 | 1.00 | 32.94 | C |
| ATOM | 2558 | CB | ARG | C | 130 | 42.496 | 119.868 | 88.325 | 1.00 | 34.08 | C |
| ATOM | 2559 | CG | ARG | C | 130 | 41.792 | 121.061 | 88.891 | 1.00 | 37.35 | C |
| ATOM | 2560 | CD | ARG | C | 130 | 40.742 | 120.618 | 89.946 | 1.00 | 40.95 | C |
| ATOM | 2561 | NE | ARG | C | 130 | 39.854 | 121.694 | 90.385 | 1.00 | 44.31 | C |
| ATOM | 2562 | CZ | ARG | C | 130 | 40.251 | 122.753 | 91.109 | 1.00 | 47.14 | C |
| ATOM | 2563 | NH1 | ARG | C | 130 | 41.541 | 122.875 | 91.485 | 1.00 | 49.43 | C |
| ATOM | 2564 | NH2 | ARG | C | 130 | 39.369 | 123.709 | 91.459 | 1.00 | 48.31 | C |
| ATOM | 2565 | C | ARG | C | 130 | 44.274 | 121.385 | 87.627 | 1.00 | 33.26 | C |
| ATOM | 2566 | O | ARG | C | 130 | 45.012 | 121.313 | 88.589 | 1.00 | 34.61 | C |
| ATOM | 2567 | N | PHE | C | 131 | 44.153 | 122.491 | 86.918 | 1.00 | 32.50 | C |
| ATOM | 2568 | CA | PHE | C | 131 | 44.871 | 123.665 | 87.345 | 1.00 | 31.73 | C |
| ATOM | 2569 | CB | PHE | C | 131 | 45.159 | 124.549 | 86.191 | 1.00 | 29.41 | C |
| ATOM | 2570 | CG | PHE | C | 131 | 46.147 | 123.980 | 85.259 | 1.00 | 28.37 | C |
| ATOM | 2571 | CD1 | PHE | C | 131 | 45.784 | 122.911 | 84.410 | 1.00 | 26.82 | C |
| ATOM | 2572 | CD2 | PHE | C | 131 | 47.437 | 124.506 | 85.198 | 1.00 | 24.53 | C |
| ATOM | 2573 | CE1 | PHE | C | 131 | 46.696 | 122.379 | 83.509 | 1.00 | 25.51 | C |
| ATOM | 2574 | CE2 | PHE | C | 131 | 48.341 | 123.985 | 84.306 | 1.00 | 26.00 | C |
| ATOM | 2575 | CZ | PHE | C | 131 | 47.977 | 122.918 | 83.453 | 1.00 | 26.06 | C |
| ATOM | 2576 | C | PHE | C | 131 | 44.127 | 124.470 | 88.361 | 1.00 | 34.03 | C |
| ATOM | 2577 | O | PHE | C | 131 | 42.946 | 124.737 | 88.208 | 1.00 | 36.47 | C |
| ATOM | 2578 | N | ASN | C | 132 | 44.827 | 124.867 | 89.409 | 1.00 | 37.26 | C |
| ATOM | 2579 | CA | ASN | C | 132 | 44.275 | 125.675 | 90.487 | 1.00 | 37.97 | C |
| ATOM | 2580 | CB | ASN | C | 132 | 44.988 | 125.334 | 91.797 | 1.00 | 39.99 | C |
| ATOM | 2581 | CG | ASN | C | 132 | 44.322 | 125.962 | 93.024 | 1.00 | 44.28 | C |
| ATOM | 2582 | OD1 | ASN | C | 132 | 43.131 | 125.746 | 93.303 | 1.00 | 47.04 | C |
| ATOM | 2583 | ND2 | ASN | C | 132 | 45.096 | 126.720 | 93.776 | 1.00 | 46.02 | C |
| ATOM | 2584 | C | ASN | C | 132 | 44.590 | 127.121 | 90.105 | 1.00 | 38.49 | C |
| ATOM | 2585 | O | ASN | C | 132 | 45.783 | 127.522 | 89.989 | 1.00 | 40.17 | C |
| ATOM | 2586 | N | ASP | C | 133 | 43.553 | 127.909 | 89.885 | 1.00 | 35.68 | C |
| ATOM | 2587 | CA | ASP | C | 133 | 43.736 | 129.311 | 89.561 | 1.00 | 34.27 | C |
| ATOM | 2588 | CB | ASP | C | 133 | 44.190 | 130.069 | 90.789 | 1.00 | 35.39 | C |
| ATOM | 2589 | CG | ASP | C | 133 | 43.822 | 131.546 | 90.717 | 1.00 | 38.00 | C |
| ATOM | 2590 | OD1 | ASP | C | 133 | 42.828 | 131.872 | 90.045 | 1.00 | 37.80 | C |
| ATOM | 2591 | OD2 | ASP | C | 133 | 44.510 | 132.391 | 91.338 | 1.00 | 40.98 | C |
| ATOM | 2592 | C | ASP | C | 133 | 44.616 | 129.730 | 88.400 | 1.00 | 32.74 | C |
| ATOM | 2593 | O | ASP | C | 133 | 45.331 | 130.725 | 88.496 | 1.00 | 31.56 | C |
| ATOM | 2594 | N | LEU | C | 134 | 44.573 | 128.992 | 87.294 | 1.00 | 31.56 | C |
| ATOM | 2595 | CA | LEU | C | 134 | 45.364 | 129.416 | 86.119 | 1.00 | 30.91 | C |
| ATOM | 2596 | CB | LEU | C | 134 | 45.109 | 128.459 | 84.934 | 1.00 | 29.53 | C |
| ATOM | 2597 | CG | LEU | C | 134 | 45.482 | 128.973 | 83.535 | 1.00 | 28.14 | C |
| ATOM | 2598 | CD1 | LEU | C | 134 | 46.980 | 129.104 | 83.411 | 1.00 | 29.33 | C |
| ATOM | 2599 | CD2 | LEU | C | 134 | 44.973 | 128.030 | 82.508 | 1.00 | 26.88 | C |
| ATOM | 2600 | C | LEU | C | 134 | 44.894 | 130.847 | 85.741 | 1.00 | 30.21 | C |
| ATOM | 2601 | O | LEU | C | 134 | 43.704 | 131.142 | 85.789 | 1.00 | 30.09 | C |
| ATOM | 2602 | N | ARG | C | 135 | 45.822 | 131.733 | 85.403 | 1.00 | 29.58 | C |
| ATOM | 2603 | CA | ARG | C | 135 | 45.474 | 133.105 | 84.984 | 1.00 | 29.40 | C |
| ATOM | 2604 | CB | ARG | C | 135 | 45.624 | 134.129 | 86.094 | 1.00 | 30.04 | C |
| ATOM | 2605 | CG | ARG | C | 135 | 44.784 | 133.842 | 87.299 | 1.00 | 33.62 | C |
| ATOM | 2606 | CD | ARG | C | 135 | 44.726 | 135.001 | 88.203 | 1.00 | 34.28 | C |
| ATOM | 2607 | NE | ARG | C | 135 | 44.351 | 134.610 | 89.548 | 1.00 | 35.57 | C |
| ATOM | 2608 | CZ | ARG | C | 135 | 44.110 | 135.504 | 90.517 | 1.00 | 37.54 | C |
| ATOM | 2609 | NH1 | ARG | C | 135 | 44.194 | 136.808 | 90.254 | 1.00 | 36.98 | C |
| ATOM | 2610 | NH2 | ARG | C | 135 | 43.842 | 135.107 | 91.749 | 1.00 | 34.63 | C |
| ATOM | 2611 | C | ARG | C | 135 | 46.314 | 133.622 | 83.857 | 1.00 | 29.56 | C |
| ATOM | 2612 | O | ARG | C | 135 | 47.540 | 133.355 | 83.774 | 1.00 | 30.47 | C |
| ATOM | 2613 | N | PHE | C | 136 | 45.661 | 134.373 | 82.986 | 1.00 | 27.71 | C |
| ATOM | 2614 | CA | PHE | C | 136 | 46.374 | 134.976 | 81.894 | 1.00 | 29.41 | C |
| ATOM | 2615 | CB | PHE | C | 136 | 45.522 | 134.935 | 80.643 | 1.00 | 28.54 | C |
| ATOM | 2616 | CG | PHE | C | 136 | 45.229 | 133.568 | 80.181 | 1.00 | 27.81 | C |
| ATOM | 2617 | CD1 | PHE | C | 136 | 44.279 | 132.800 | 80.819 | 1.00 | 28.56 | C |
| ATOM | 2618 | CD2 | PHE | C | 136 | 45.925 | 133.027 | 79.124 | 1.00 | 25.86 | C |
| ATOM | 2619 | CE1 | PHE | C | 136 | 44.030 | 131.486 | 80.392 | 1.00 | 28.56 | C |
| ATOM | 2620 | CE2 | PHE | C | 136 | 45.686 | 131.750 | 78.699 | 1.00 | 26.19 | C |
| ATOM | 2621 | CZ | PHE | C | 136 | 44.749 | 130.970 | 79.314 | 1.00 | 26.97 | C |
| ATOM | 2622 | C | PHE | C | 136 | 46.694 | 136.432 | 82.258 | 1.00 | 30.80 | C |
| ATOM | 2623 | O | PHE | C | 136 | 45.776 | 137.239 | 82.474 | 1.00 | 31.28 | C |
| ATOM | 2624 | N | VAL | C | 137 | 47.979 | 136.779 | 82.327 | 1.00 | 31.42 | C |
| ATOM | 2625 | CA | VAL | C | 137 | 48.365 | 138.162 | 82.666 | 1.00 | 32.80 | C |
| ATOM | 2626 | CB | VAL | C | 137 | 49.603 | 138.192 | 83.566 | 1.00 | 32.75 | C |
| ATOM | 2627 | CG1 | VAL | C | 137 | 49.944 | 139.604 | 83.959 | 1.00 | 29.80 | C |
| ATOM | 2628 | CG2 | VAL | C | 137 | 49.336 | 137.354 | 84.781 | 1.00 | 32.80 | C |
| ATOM | 2629 | C | VAL | C | 137 | 48.659 | 138.965 | 81.428 | 1.00 | 33.01 | C |
| ATOM | 2630 | O | VAL | C | 137 | 48.211 | 140.074 | 81.314 | 1.00 | 35.16 | C |
| ATOM | 2631 | N | GLY | C | 138 | 49.423 | 138.414 | 80.512 | 1.00 | 33.06 | C |
| ATOM | 2632 | CA | GLY | C | 138 | 49.682 | 139.117 | 79.272 | 1.00 | 34.89 | C |
| ATOM | 2633 | C | GLY | C | 138 | 48.453 | 139.326 | 78.378 | 1.00 | 35.80 | C |
| ATOM | 2634 | O | GLY | C | 138 | 47.496 | 138.544 | 78.375 | 1.00 | 35.47 | C |
| ATOM | 2635 | N | ARG | C | 139 | 48.474 | 140.385 | 77.585 | 1.00 | 36.48 | C |
| ATOM | 2636 | CA | ARG | C | 139 | 47.340 | 140.649 | 76.751 | 1.00 | 37.26 | C |
| ATOM | 2637 | CB | ARG | C | 139 | 47.188 | 142.140 | 76.579 | 1.00 | 41.59 | C |
| ATOM | 2638 | CG | ARG | C | 139 | 47.470 | 142.984 | 77.821 | 1.00 | 46.37 | C |
| ATOM | 2639 | CD | ARG | C | 139 | 48.131 | 144.333 | 77.325 | 1.00 | 51.78 | C |
| ATOM | 2640 | NE | ARG | C | 139 | 48.526 | 145.251 | 78.399 | 1.00 | 57.91 | C |
| ATOM | 2641 | CZ | ARG | C | 139 | 48.859 | 144.886 | 79.654 | 1.00 | 61.47 | C |
| ATOM | 2642 | NH1 | ARG | C | 139 | 48.864 | 143.584 | 80.065 | 1.00 | 61.84 | C |
| ATOM | 2643 | NH2 | ARG | C | 139 | 49.163 | 145.850 | 80.531 | 1.00 | 62.11 | C |
| ATOM | 2644 | C | ARG | C | 139 | 47.516 | 139.986 | 75.412 | 1.00 | 36.42 | C |
| ATOM | 2645 | O | ARG | C | 139 | 48.647 | 139.746 | 74.987 | 1.00 | 36.92 | C |
| ATOM | 2646 | N | SER | C | 140 | 46.404 | 139.698 | 74.739 | 1.00 | 35.20 | C |
| ATOM | 2647 | CA | SER | C | 140 | 46.482 | 139.080 | 73.429 | 1.00 | 34.67 | C |
| ATOM | 2648 | CB | SER | C | 140 | 45.457 | 137.967 | 73.315 | 1.00 | 35.18 | C |
| ATOM | 2649 | OG | SER | C | 140 | 44.145 | 138.474 | 73.152 | 1.00 | 35.11 | C |
| ATOM | 2650 | C | SER | C | 140 | 46.328 | 140.036 | 72.233 | 1.00 | 34.38 | C |
| ATOM | 2651 | O | SER | C | 140 | 46.619 | 139.637 | 71.134 | 1.00 | 35.00 | C |
| ATOM | 2652 | N | GLY | C | 141 | 45.882 | 141.279 | 72.426 | 1.00 | 33.71 | C |
| ATOM | 2653 | CA | GLY | C | 141 | 45.730 | 142.184 | 71.302 | 1.00 | 33.85 | C |
| ATOM | 2654 | C | GLY | C | 141 | 44.277 | 142.314 | 70.812 | 1.00 | 35.72 | C |
| ATOM | 2655 | O | GLY | C | 141 | 43.521 | 141.322 | 70.691 | 1.00 | 35.44 | C |
| ATOM | 2656 | N | ARG | C | 142 | 43.871 | 143.550 | 70.519 | 1.00 | 36.75 | C |
| ATOM | 2657 | CA | ARG | C | 142 | 42.513 | 143.850 | 70.003 | 1.00 | 37.71 | C |
| ATOM | 2658 | CB | ARG | C | 142 | 42.493 | 145.233 | 69.346 | 1.00 | 37.84 | C |
| ATOM | 2659 | CG | ARG | C | 142 | 41.135 | 145.799 | 69.143 | 1.00 | 39.53 | C |
| ATOM | 2660 | CD | ARG | C | 142 | 41.224 | 146.930 | 68.139 | 1.00 | 41.58 | C |
| ATOM | 2661 | NE | ARG | C | 142 | 39.908 | 147.440 | 67.762 | 1.00 | 45.71 | C |
| ATOM | 2662 | CZ | ARG | C | 142 | 39.192 | 148.292 | 68.488 | 1.00 | 48.33 | C |
| ATOM | 2663 | NH1 | ARG | C | 142 | 39.666 | 148.756 | 69.646 | 1.00 | 48.76 | C |
| ATOM | 2664 | NH2 | ARG | C | 142 | 37.985 | 148.667 | 68.057 | 1.00 | 50.63 | C |
| ATOM | 2665 | C | ARG | C | 142 | 42.118 | 142.801 | 68.962 | 1.00 | 37.32 | C |
| ATOM | 2666 | O | ARG | C | 142 | 42.936 | 142.433 | 68.098 | 1.00 | 37.71 | C |
| ATOM | 2667 | N | GLY | C | 143 | 40.874 | 142.330 | 69.045 | 1.00 | 36.31 | C |
| ATOM | 2668 | CA | GLY | C | 143 | 40.401 | 141.296 | 68.140 | 1.00 | 35.43 | C |
| ATOM | 2669 | C | GLY | C | 143 | 41.319 | 140.078 | 68.019 | 1.00 | 34.77 | C |
| ATOM | 2670 | O | GLY | C | 143 | 41.326 | 139.418 | 67.011 | 1.00 | 35.52 | C |
| ATOM | 2671 | N | LYS | C | 144 | 42.108 | 139.755 | 69.022 | 1.00 | 34.49 | C |
| ATOM | 2672 | CA | LYS | C | 144 | 42.961 | 138.593 | 68.870 | 1.00 | 35.15 | C |
| ATOM | 2673 | CB | LYS | C | 144 | 44.393 | 139.049 | 68.665 | 1.00 | 36.12 | C |
| ATOM | 2674 | CG | LYS | C | 144 | 44.595 | 139.678 | 67.319 | 1.00 | 38.14 | C |
| ATOM | 2675 | CD | LYS | C | 144 | 46.071 | 139.974 | 67.107 | 1.00 | 39.77 | C |
| ATOM | 2676 | CE | LYS | C | 144 | 46.350 | 140.376 | 65.658 | 1.00 | 40.19 | C |
| ATOM | 2677 | NZ | LYS | C | 144 | 47.797 | 140.681 | 65.402 | 1.00 | 38.89 | C |
| ATOM | 2678 | C | LYS | C | 144 | 42.890 | 137.578 | 70.026 | 1.00 | 34.25 | C |
| ATOM | 2679 | O | LYS | C | 144 | 42.466 | 137.895 | 71.138 | 1.00 | 34.48 | C |
| ATOM | 2680 | N | SER | C | 145 | 43.288 | 136.340 | 69.774 | 1.00 | 32.44 | C |
| ATOM | 2681 | CA | SER | C | 145 | 43.215 | 135.418 | 70.857 | 1.00 | 31.64 | C |
| ATOM | 2682 | CB | SER | C | 145 | 41.992 | 134.559 | 70.728 | 1.00 | 30.06 | C |
| ATOM | 2683 | OG | SER | C | 145 | 42.275 | 133.610 | 69.781 | 1.00 | 34.26 | C |
| ATOM | 2684 | C | SER | C | 145 | 44.493 | 134.624 | 70.927 | 1.00 | 31.05 | C |
| ATOM | 2685 | O | SER | C | 145 | 45.246 | 134.567 | 69.984 | 1.00 | 30.76 | C |
| ATOM | 2686 | N | PHE | C | 146 | 44.772 | 134.072 | 72.096 | 1.00 | 31.20 | C |
| ATOM | 2687 | CA | PHE | C | 146 | 45.994 | 133.329 | 72.293 | 1.00 | 31.16 | C |
| ATOM | 2688 | CB | PHE | C | 146 | 46.348 | 133.261 | 73.796 | 1.00 | 32.37 | C |
| ATOM | 2689 | CG | PHE | C | 146 | 46.995 | 134.517 | 74.369 | 1.00 | 31.15 | C |
| ATOM | 2690 | CD1 | PHE | C | 146 | 46.510 | 135.088 | 75.551 | 1.00 | 30.06 | C |
| ATOM | 2691 | CD2 | PHE | C | 146 | 48.088 | 135.099 | 73.756 | 1.00 | 30.78 | C |
| ATOM | 2692 | CE1 | PHE | C | 146 | 47.090 | 136.206 | 76.096 | 1.00 | 28.41 | C |
| ATOM | 2693 | CE2 | PHE | C | 146 | 48.672 | 136.218 | 74.300 | 1.00 | 30.83 | C |
| ATOM | 2694 | CZ | PHE | C | 146 | 48.153 | 136.777 | 75.489 | 1.00 | 28.59 | C |
| ATOM | 2695 | C | PHE | C | 146 | 45.867 | 131.908 | 71.794 | 1.00 | 31.41 | C |
| ATOM | 2696 | O | PHE | C | 146 | 44.805 | 131.335 | 71.717 | 1.00 | 31.98 | C |
| ATOM | 2697 | N | THR | C | 147 | 46.983 | 131.325 | 71.449 | 1.00 | 32.53 | C |
| ATOM | 2698 | CA | THR | C | 147 | 46.984 | 129.940 | 71.046 | 1.00 | 32.35 | C |
| ATOM | 2699 | CB | THR | C | 147 | 47.828 | 129.762 | 69.782 | 1.00 | 32.69 | C |
| ATOM | 2700 | OG1 | THR | C | 147 | 47.076 | 130.261 | 68.671 | 1.00 | 35.35 | C |
| ATOM | 2701 | CG2 | THR | C | 147 | 48.203 | 128.291 | 69.559 | 1.00 | 30.16 | C |
| ATOM | 2702 | C | THR | C | 147 | 47.675 | 129.295 | 72.247 | 1.00 | 32.47 | C |
| ATOM | 2703 | O | THR | C | 147 | 48.768 | 129.726 | 72.650 | 1.00 | 33.26 | C |
| ATOM | 2704 | N | LEU | C | 148 | 47.054 | 128.291 | 72.840 | 1.00 | 31.00 | C |
| ATOM | 2705 | CA | LEU | C | 148 | 47.707 | 127.667 | 73.977 | 1.00 | 31.17 | C |
| ATOM | 2706 | CB | LEU | C | 148 | 46.696 | 127.100 | 74.967 | 1.00 | 29.65 | C |
| ATOM | 2707 | CG | LEU | C | 148 | 45.657 | 128.021 | 75.532 | 1.00 | 29.82 | C |
| ATOM | 2708 | CD1 | LEU | C | 148 | 44.960 | 127.252 | 76.602 | 1.00 | 29.31 | C |
| ATOM | 2709 | CD2 | LEU | C | 148 | 46.295 | 129.286 | 76.081 | 1.00 | 28.30 | C |
| ATOM | 2710 | C | LEU | C | 148 | 48.576 | 126.487 | 73.595 | 1.00 | 31.90 | C |
| ATOM | 2711 | O | LEU | C | 148 | 48.276 | 125.756 | 72.621 | 1.00 | 31.95 | C |
| ATOM | 2712 | N | THR | C | 149 | 49.629 | 126.282 | 74.393 | 1.00 | 31.69 | C |
| ATOM | 2713 | CA | THR | C | 149 | 50.501 | 125.111 | 74.239 | 1.00 | 30.24 | C |
| ATOM | 2714 | CB | THR | C | 149 | 51.915 | 125.477 | 73.970 | 1.00 | 29.58 | C |
| ATOM | 2715 | OG1 | THR | C | 149 | 52.016 | 126.061 | 72.655 | 1.00 | 30.84 | C |
| ATOM | 2716 | CG2 | THR | C | 149 | 52.712 | 124.222 | 74.011 | 1.00 | 29.46 | C |
| ATOM | 2717 | C | THR | C | 149 | 50.413 | 124.315 | 75.539 | 1.00 | 29.25 | C |
| ATOM | 2718 | O | THR | C | 149 | 50.764 | 124.813 | 76.587 | 1.00 | 29.68 | C |
| ATOM | 2719 | N | ILE | C | 150 | 49.835 | 123.119 | 75.483 | 1.00 | 28.55 | C |
| ATOM | 2720 | CA | ILE | C | 150 | 49.678 | 122.282 | 76.668 | 1.00 | 27.65 | C |
| ATOM | 2721 | CB | ILE | C | 150 | 48.298 | 121.624 | 76.739 | 1.00 | 25.88 | C |
| ATOM | 2722 | CG2 | ILE | C | 150 | 48.262 | 120.672 | 77.884 | 1.00 | 22.25 | C |
| ATOM | 2723 | CG1 | ILE | C | 150 | 47.190 | 122.671 | 76.891 | 1.00 | 24.98 | C |
| ATOM | 2724 | CD1 | ILE | C | 150 | 45.884 | 122.133 | 76.483 | 1.00 | 21.01 | C |
| ATOM | 2725 | C | ILE | C | 150 | 50.710 | 121.194 | 76.466 | 1.00 | 30.46 | C |
| ATOM | 2726 | O | ILE | C | 150 | 50.695 | 120.505 | 75.424 | 1.00 | 32.59 | C |
| ATOM | 2727 | N | THR | C | 151 | 51.626 | 121.077 | 77.432 | 1.00 | 31.62 | C |
| ATOM | 2728 | CA | THR | C | 151 | 52.718 | 120.125 | 77.401 | 1.00 | 31.11 | C |
| ATOM | 2729 | CB | THR | C | 151 | 54.072 | 120.834 | 77.548 | 1.00 | 31.95 | C |
| ATOM | 2730 | OG1 | THR | C | 151 | 54.145 | 121.912 | 76.607 | 1.00 | 34.33 | C |
| ATOM | 2731 | CG2 | THR | C | 151 | 55.228 | 119.884 | 77.264 | 1.00 | 30.94 | C |
| ATOM | 2732 | C | THR | C | 151 | 52.540 | 119.173 | 78.556 | 1.00 | 32.17 | C |
| ATOM | 2733 | O | THR | C | 151 | 52.348 | 119.597 | 79.732 | 1.00 | 32.24 | C |
| ATOM | 2734 | N | VAL | C | 152 | 52.534 | 117.882 | 78.213 | 1.00 | 31.21 | C |
| ATOM | 2735 | CA | VAL | C | 152 | 52.407 | 116.844 | 79.207 | 1.00 | 31.12 | C |
| ATOM | 2736 | CB | VAL | C | 152 | 51.314 | 115.891 | 78.847 | 1.00 | 29.96 | C |
| ATOM | 2737 | CG1 | VAL | C | 152 | 51.378 | 114.639 | 79.748 | 1.00 | 27.76 | C |
| ATOM | 2738 | CG2 | VAL | C | 152 | 49.996 | 116.600 | 78.968 | 1.00 | 28.60 | C |
| ATOM | 2739 | C | VAL | C | 152 | 53.779 | 116.174 | 79.190 | 1.00 | 34.28 | C |
| ATOM | 2740 | O | VAL | C | 152 | 54.230 | 115.631 | 78.155 | 1.00 | 34.65 | C |
| ATOM | 2741 | N | PHE | C | 153 | 54.451 | 116.250 | 80.341 | 1.00 | 35.20 | C |
| ATOM | 2742 | CA | PHE | C | 153 | 55.811 | 115.766 | 80.477 | 1.00 | 36.00 | C |
| ATOM | 2743 | CB | PHE | C | 153 | 56.550 | 116.613 | 81.538 | 1.00 | 34.78 | C |
| ATOM | 2744 | CG | PHE | C | 153 | 56.792 | 118.047 | 81.128 | 1.00 | 33.63 | C |
| ATOM | 2745 | CD1 | PHE | C | 153 | 55.801 | 119.029 | 81.294 | 1.00 | 33.83 | C |
| ATOM | 2746 | CD2 | PHE | C | 153 | 57.985 | 118.420 | 80.530 | 1.00 | 32.22 | C |
| ATOM | 2747 | CE1 | PHE | C | 153 | 55.992 | 120.376 | 80.860 | 1.00 | 31.42 | C |
| ATOM | 2748 | CE2 | PHE | C | 153 | 58.191 | 119.764 | 80.086 | 1.00 | 31.60 | C |
| ATOM | 2749 | CZ | PHE | C | 153 | 57.193 | 120.738 | 80.250 | 1.00 | 30.19 | C |
| ATOM | 2750 | C | PHE | C | 153 | 56.059 | 114.279 | 80.724 | 1.00 | 36.78 | C |
| ATOM | 2751 | O | PHE | C | 153 | 56.421 | 113.853 | 81.802 | 1.00 | 37.42 | C |
| ATOM | 2752 | N | THR | C | 154 | 55.882 | 113.505 | 79.674 | 1.00 | 39.01 | C |
| ATOM | 2753 | CA | THR | C | 154 | 56.155 | 112.073 | 79.652 | 1.00 | 39.76 | C |
| ATOM | 2754 | CB | THR | C | 154 | 55.094 | 111.379 | 78.803 | 1.00 | 38.18 | C |
| ATOM | 2755 | OG1 | THR | C | 154 | 54.871 | 112.142 | 77.600 | 1.00 | 37.55 | C |
| ATOM | 2756 | CG2 | THR | C | 154 | 53.799 | 111.246 | 79.562 | 1.00 | 36.81 | C |
| ATOM | 2757 | C | THR | C | 154 | 57.507 | 112.085 | 78.888 | 1.00 | 42.45 | C |
| ATOM | 2758 | O | THR | C | 154 | 58.004 | 113.185 | 78.528 | 1.00 | 43.06 | C |
| ATOM | 2759 | N | ASN | C | 155 | 58.122 | 110.925 | 78.644 | 1.00 | 44.92 | C |
| ATOM | 2760 | CA | ASN | C | 155 | 59.370 | 110.906 | 77.850 | 1.00 | 47.63 | C |
| ATOM | 2761 | CB | ASN | C | 155 | 60.451 | 110.177 | 78.583 | 1.00 | 52.18 | C |
| ATOM | 2762 | CG | ASN | C | 155 | 60.559 | 110.648 | 79.988 | 1.00 | 57.17 | C |
| ATOM | 2763 | OD1 | ASN | C | 155 | 60.930 | 111.829 | 80.240 | 1.00 | 59.63 | C |
| ATOM | 2764 | ND2 | ASN | C | 155 | 60.207 | 109.759 | 80.948 | 1.00 | 59.24 | C |
| ATOM | 2765 | C | ASN | C | 155 | 59.142 | 110.162 | 76.573 | 1.00 | 46.46 | C |
| ATOM | 2766 | O | ASN | C | 155 | 58.970 | 108.952 | 76.611 | 1.00 | 48.77 | C |
| ATOM | 2767 | N | CPR | C | 156 | 59.088 | 110.862 | 75.438 | 1.00 | 44.14 | C |
| ATOM | 2768 | CD | CPR | C | 156 | 58.882 | 110.310 | 74.088 | 1.00 | 43.00 | C |
| ATOM | 2769 | CA | CPR | C | 156 | 59.232 | 112.306 | 75.369 | 1.00 | 42.57 | C |
| ATOM | 2770 | CB | CPR | C | 156 | 59.599 | 112.523 | 73.926 | 1.00 | 43.09 | C |
| ATOM | 2771 | CG | CPR | C | 156 | 58.691 | 111.566 | 73.266 | 1.00 | 42.89 | C |
| ATOM | 2772 | C | CPR | C | 156 | 57.952 | 113.042 | 75.710 | 1.00 | 40.74 | C |
| ATOM | 2773 | O | CPR | C | 156 | 56.889 | 112.477 | 75.857 | 1.00 | 41.66 | C |
| ATOM | 2774 | N | PRO | C | 157 | 58.043 | 114.338 | 75.815 | 1.00 | 39.34 | C |
| ATOM | 2775 | CD | PRO | C | 157 | 59.222 | 115.203 | 75.657 | 1.00 | 38.18 | C |
| ATOM | 2776 | CA | PRO | C | 157 | 56.840 | 115.100 | 76.143 | 1.00 | 39.29 | C |
| ATOM | 2777 | CB | PRO | C | 157 | 57.377 | 116.546 | 76.253 | 1.00 | 39.14 | C |
| ATOM | 2778 | CG | PRO | C | 157 | 58.898 | 116.339 | 76.550 | 1.00 | 37.70 | C |
| ATOM | 2779 | C | PRO | C | 157 | 55.789 | 114.998 | 75.049 | 1.00 | 37.95 | C |
| ATOM | 2780 | O | PRO | C | 157 | 56.126 | 114.817 | 73.890 | 1.00 | 38.79 | C |
| ATOM | 2781 | N | GLN | C | 158 | 54.520 | 115.136 | 75.410 | 1.00 | 37.52 | C |
| ATOM | 2782 | CA | GLN | C | 158 | 53.434 | 115.159 | 74.401 | 1.00 | 35.07 | C |
| ATOM | 2783 | CB | GLN | C | 158 | 52.317 | 114.186 | 74.754 | 1.00 | 35.50 | C |
| ATOM | 2784 | CG | GLN | C | 158 | 52.781 | 112.767 | 74.709 | 1.00 | 34.35 | C |
| ATOM | 2785 | CD | GLN | C | 158 | 51.695 | 111.805 | 75.081 | 1.00 | 36.62 | C |
| ATOM | 2786 | OE1 | GLN | C | 158 | 50.677 | 111.693 | 74.386 | 1.00 | 36.49 | C |
| ATOM | 2787 | NE2 | GLN | C | 158 | 51.892 | 111.099 | 76.193 | 1.00 | 35.38 | C |
| ATOM | 2788 | C | GLN | C | 158 | 52.936 | 116.588 | 74.479 | 1.00 | 33.32 | C |
| ATOM | 2789 | O | GLN | C | 158 | 52.788 | 117.142 | 75.589 | 1.00 | 32.85 | C |
| ATOM | 2790 | N | VAL | C | 159 | 52.720 | 117.194 | 73.317 | 1.00 | 31.22 | C |
| ATOM | 2791 | CA | VAL | C | 159 | 52.301 | 118.569 | 73.264 | 1.00 | 29.12 | C |
| ATOM | 2792 | CB | VAL | C | 159 | 53.397 | 119.416 | 72.597 | 1.00 | 28.28 | C |
| ATOM | 2793 | CG1 | VAL | C | 159 | 53.008 | 120.892 | 72.600 | 1.00 | 24.72 | C |
| ATOM | 2794 | CG2 | VAL | C | 159 | 54.735 | 119.238 | 73.355 | 1.00 | 24.46 | C |
| ATOM | 2795 | C | VAL | C | 159 | 51.033 | 118.697 | 72.500 | 1.00 | 30.16 | C |
| ATOM | 2796 | O | VAL | C | 159 | 50.868 | 118.038 | 71.502 | 1.00 | 29.55 | C |
| ATOM | 2797 | N | ALA | C | 160 | 50.138 | 119.559 | 72.984 | 1.00 | 31.65 | C |
| ATOM | 2798 | CA | ALA | C | 160 | 48.838 | 119.792 | 72.363 | 1.00 | 32.47 | C |
| ATOM | 2799 | CB | ALA | C | 160 | 47.738 | 119.130 | 73.197 | 1.00 | 31.05 | C |
| ATOM | 2800 | C | ALA | C | 160 | 48.555 | 121.260 | 72.318 | 1.00 | 33.73 | C |
| ATOM | 2801 | O | ALA | C | 160 | 48.628 | 121.934 | 73.357 | 1.00 | 34.40 | C |
| ATOM | 2802 | N | THR | C | 161 | 48.224 | 121.771 | 71.141 | 1.00 | 34.55 | C |
| ATOM | 2803 | CA | THR | C | 161 | 47.852 | 123.180 | 71.054 | 1.00 | 35.53 | C |
| ATOM | 2804 | CB | THR | C | 161 | 48.476 | 123.861 | 69.822 | 1.00 | 33.32 | C |
| ATOM | 2805 | OG1 | THR | C | 161 | 48.067 | 123.169 | 68.658 | 1.00 | 34.69 | C |
| ATOM | 2806 | CG2 | THR | C | 161 | 49.997 | 123.806 | 69.907 | 1.00 | 33.77 | C |
| ATOM | 2807 | C | THR | C | 161 | 46.296 | 123.302 | 71.041 | 1.00 | 36.01 | C |
| ATOM | 2808 | O | THR | C | 161 | 45.560 | 122.328 | 70.840 | 1.00 | 36.48 | C |
| ATOM | 2809 | N | TYR | C | 162 | 45.816 | 124.495 | 71.345 | 1.00 | 36.41 | C |
| ATOM | 2810 | CA | TYR | C | 162 | 44.418 | 124.842 | 71.355 | 1.00 | 35.42 | C |
| ATOM | 2811 | CB | TYR | C | 162 | 44.006 | 124.834 | 72.786 | 1.00 | 36.91 | C |
| ATOM | 2812 | CG | TYR | C | 162 | 42.585 | 125.215 | 73.074 | 1.00 | 40.97 | C |
| ATOM | 2813 | CD1 | TYR | C | 162 | 42.153 | 125.338 | 74.415 | 1.00 | 42.63 | C |
| ATOM | 2814 | CE1 | TYR | C | 162 | 40.850 | 125.751 | 74.750 | 1.00 | 44.91 | C |
| ATOM | 2815 | CD2 | TYR | C | 162 | 41.684 | 125.506 | 72.057 | 1.00 | 41.57 | C |
| ATOM | 2816 | CE2 | TYR | C | 162 | 40.384 | 125.919 | 72.351 | 1.00 | 45.00 | C |
| ATOM | 2817 | CZ | TYR | C | 162 | 39.958 | 126.050 | 73.710 | 1.00 | 47.80 | C |
| ATOM | 2818 | OH | TYR | C | 162 | 38.678 | 126.519 | 74.059 | 1.00 | 49.20 | C |
| ATOM | 2819 | C | TYR | C | 162 | 44.488 | 126.254 | 70.741 | 1.00 | 35.83 | C |
| ATOM | 2820 | O | TYR | C | 162 | 44.913 | 127.214 | 71.386 | 1.00 | 36.47 | C |
| ATOM | 2821 | N | HIS | C | 163 | 44.158 | 126.358 | 69.466 | 1.00 | 36.31 | C |
| ATOM | 2822 | CA | HIS | C | 163 | 44.264 | 127.625 | 68.743 | 1.00 | 38.00 | C |
| ATOM | 2823 | CB | HIS | C | 163 | 44.388 | 127.348 | 67.223 | 1.00 | 39.67 | C |
| ATOM | 2824 | CG | HIS | C | 163 | 45.794 | 127.075 | 66.756 | 1.00 | 41.87 | C |
| ATOM | 2825 | CD2 | HIS | C | 163 | 46.762 | 127.912 | 66.292 | 1.00 | 43.04 | C |
| ATOM | 2826 | ND1 | HIS | C | 163 | 46.394 | 125.838 | 66.876 | 1.00 | 42.34 | C |
| ATOM | 2827 | CE1 | HIS | C | 163 | 47.666 | 125.921 | 66.526 | 1.00 | 42.13 | C |
| ATOM | 2828 | NE2 | HIS | C | 163 | 47.917 | 127.169 | 66.169 | 1.00 | 44.05 | C |
| ATOM | 2829 | C | HIS | C | 163 | 43.073 | 128.542 | 69.031 | 1.00 | 38.22 | C |
| ATOM | 2830 | O | HIS | C | 163 | 41.972 | 128.047 | 69.292 | 1.00 | 37.27 | C |
| ATOM | 2831 | N | ARG | C | 164 | 43.313 | 129.865 | 69.032 | 1.00 | 39.32 | C |
| ATOM | 2832 | CA | ARG | C | 164 | 42.244 | 130.862 | 69.262 | 1.00 | 39.68 | C |
| ATOM | 2833 | CB | ARG | C | 164 | 41.336 | 130.949 | 68.022 | 1.00 | 42.31 | C |
| ATOM | 2834 | CG | ARG | C | 164 | 42.029 | 131.581 | 66.789 | 1.00 | 46.60 | C |
| ATOM | 2835 | CD | ARG | C | 164 | 41.241 | 131.348 | 65.484 | 1.00 | 52.63 | C |
| ATOM | 2836 | NE | ARG | C | 164 | 41.913 | 130.302 | 64.699 | 1.00 | 57.97 | C |
| ATOM | 2837 | CZ | ARG | C | 164 | 41.464 | 129.053 | 64.497 | 1.00 | 58.83 | C |
| ATOM | 2838 | NH1 | ARG | C | 164 | 40.294 | 128.631 | 64.996 | 1.00 | 58.95 | C |
| ATOM | 2839 | NH2 | ARG | C | 164 | 42.238 | 128.195 | 63.835 | 1.00 | 59.81 | C |
| ATOM | 2840 | C | ARG | C | 164 | 41.415 | 130.462 | 70.455 | 1.00 | 38.31 | C |
| ATOM | 2841 | O | ARG | C | 164 | 40.180 | 130.472 | 70.415 | 1.00 | 37.61 | C |
| ATOM | 2842 | N | ALA | C | 165 | 42.112 | 130.124 | 71.532 | 1.00 | 37.92 | C |
| ATOM | 2843 | CA | ALA | C | 165 | 41.454 | 129.638 | 72.734 | 1.00 | 36.34 | C |
| ATOM | 2844 | CB | ALA | C | 165 | 42.408 | 128.791 | 73.538 | 1.00 | 35.79 | C |
| ATOM | 2845 | C | ALA | C | 165 | 40.889 | 130.722 | 73.591 | 1.00 | 35.29 | C |
| ATOM | 2846 | O | ALA | C | 165 | 39.857 | 130.510 | 74.216 | 1.00 | 36.44 | C |
| ATOM | 2847 | N | ILE | C | 166 | 41.526 | 131.891 | 73.625 | 1.00 | 32.84 | C |
| ATOM | 2848 | CA | ILE | C | 166 | 40.999 | 132.937 | 74.490 | 1.00 | 30.51 | C |
| ATOM | 2849 | CB | ILE | C | 166 | 41.254 | 132.546 | 75.949 | 1.00 | 30.91 | C |
| ATOM | 2850 | CG2 | ILE | C | 166 | 42.765 | 132.447 | 76.230 | 1.00 | 28.33 | C |
| ATOM | 2851 | CG1 | ILE | C | 166 | 40.621 | 133.563 | 76.869 | 1.00 | 31.29 | C |
| ATOM | 2852 | CD1 | ILE | C | 166 | 40.779 | 133.193 | 78.332 | 1.00 | 31.89 | C |
| ATOM | 2853 | C | ILE | C | 166 | 41.621 | 134.294 | 74.219 | 1.00 | 29.92 | C |
| ATOM | 2854 | O | ILE | C | 166 | 42.814 | 134.398 | 73.903 | 1.00 | 29.40 | C |
| ATOM | 2855 | N | LYS | C | 167 | 40.798 | 135.334 | 74.294 | 1.00 | 29.76 | C |
| ATOM | 2856 | CA | LYS | C | 167 | 41.267 | 136.715 | 74.100 | 1.00 | 30.32 | C |
| ATOM | 2857 | CB | LYS | C | 167 | 40.309 | 137.543 | 73.286 | 1.00 | 29.05 | C |
| ATOM | 2858 | CG | LYS | C | 167 | 40.679 | 138.994 | 73.335 | 1.00 | 30.41 | C |
| ATOM | 2859 | CD | LYS | C | 167 | 39.942 | 139.793 | 72.302 | 1.00 | 30.19 | C |
| ATOM | 2860 | CE | LYS | C | 167 | 38.446 | 139.884 | 72.592 | 1.00 | 30.18 | C |
| ATOM | 2861 | NZ | LYS | C | 167 | 37.793 | 140.832 | 71.637 | 1.00 | 31.20 | C |
| ATOM | 2862 | C | LYS | C | 167 | 41.362 | 137.340 | 75.488 | 1.00 | 31.85 | C |
| ATOM | 2863 | O | LYS | C | 167 | 40.400 | 137.259 | 76.296 | 1.00 | 32.58 | C |
| ATOM | 2864 | N | ILE | C | 168 | 42.525 | 137.915 | 75.780 | 1.00 | 31.88 | C |
| ATOM | 2865 | CA | ILE | C | 168 | 42.772 | 138.506 | 77.076 | 1.00 | 32.66 | C |
| ATOM | 2866 | CB | ILE | C | 168 | 44.004 | 137.884 | 77.757 | 1.00 | 31.38 | C |
| ATOM | 2867 | CG2 | ILE | C | 168 | 44.334 | 138.687 | 79.002 | 1.00 | 30.67 | C |
| ATOM | 2868 | CG1 | ILE | C | 168 | 43.715 | 136.432 | 78.142 | 1.00 | 29.73 | C |
| ATOM | 2869 | CD1 | ILE | C | 168 | 42.512 | 136.301 | 79.087 | 1.00 | 26.31 | C |
| ATOM | 2870 | C | ILE | C | 168 | 42.985 | 140.004 | 76.932 | 1.00 | 34.54 | C |
| ATOM | 2871 | O | ILE | C | 168 | 43.883 | 140.482 | 76.197 | 1.00 | 34.67 | C |
| ATOM | 2872 | N | THR | C | 169 | 42.150 | 140.753 | 77.638 | 1.00 | 34.36 | C |
| ATOM | 2873 | CA | THR | C | 169 | 42.239 | 142.212 | 77.563 | 1.00 | 33.81 | C |
| ATOM | 2874 | CB | THR | C | 169 | 40.965 | 142.850 | 76.901 | 1.00 | 31.13 | C |
| ATOM | 2875 | OG1 | THR | C | 169 | 39.805 | 142.512 | 77.687 | 1.00 | 26.90 | C |
| ATOM | 2876 | CG2 | THR | C | 169 | 40.792 | 142.343 | 75.484 | 1.00 | 31.32 | C |
| ATOM | 2877 | C | THR | C | 169 | 42.328 | 142.732 | 78.979 | 1.00 | 34.13 | C |
| ATOM | 2878 | O | THR | C | 169 | 42.035 | 142.005 | 79.925 | 1.00 | 33.49 | C |
| ATOM | 2879 | N | VAL | C | 170 | 42.687 | 144.006 | 79.086 | 1.00 | 34.51 | C |
| ATOM | 2880 | CA | VAL | C | 170 | 42.847 | 144.676 | 80.332 | 1.00 | 35.80 | C |
| ATOM | 2881 | CB | VAL | C | 170 | 43.479 | 146.008 | 79.974 | 1.00 | 37.64 | C |
| ATOM | 2882 | CG1 | VAL | C | 170 | 42.883 | 147.160 | 80.774 | 1.00 | 38.34 | C |
| ATOM | 2883 | CG2 | VAL | C | 170 | 44.991 | 145.878 | 80.155 | 1.00 | 36.90 | C |
| ATOM | 2884 | C | VAL | C | 170 | 41.518 | 144.782 | 81.102 | 1.00 | 36.60 | C |
| ATOM | 2885 | O | VAL | C | 170 | 41.470 | 144.687 | 82.336 | 1.00 | 37.63 | C |
| ATOM | 2886 | N | ASP | C | 171 | 40.429 | 144.943 | 80.372 | 1.00 | 36.48 | C |
| ATOM | 2887 | CA | ASP | C | 171 | 39.134 | 145.050 | 80.989 | 1.00 | 36.47 | C |
| ATOM | 2888 | CB | ASP | C | 171 | 38.312 | 146.091 | 80.257 | 1.00 | 37.11 | C |
| ATOM | 2889 | CG | ASP | C | 171 | 38.745 | 147.528 | 80.587 | 1.00 | 38.94 | C |
| ATOM | 2890 | OD1 | ASP | C | 171 | 39.804 | 147.766 | 81.236 | 1.00 | 37.63 | C |
| ATOM | 2891 | OD2 | ASP | C | 171 | 37.995 | 148.428 | 80.162 | 1.00 | 41.39 | C |
| ATOM | 2892 | C | ASP | C | 171 | 38.343 | 143.767 | 81.017 | 1.00 | 37.66 | C |
| ATOM | 2893 | O | ASP | C | 171 | 37.557 | 143.526 | 81.934 | 1.00 | 37.40 | C |
| ATOM | 2894 | N | GLY | C | 172 | 38.543 | 142.930 | 80.016 | 1.00 | 38.41 | C |
| ATOM | 2895 | CA | GLY | C | 172 | 37.757 | 141.728 | 79.951 | 1.00 | 38.59 | C |
| ATOM | 2896 | C | GLY | C | 172 | 36.406 | 142.167 | 79.381 | 1.00 | 39.33 | C |
| ATOM | 2897 | O | GLY | C | 172 | 36.190 | 143.346 | 79.075 | 1.00 | 36.51 | C |
| ATOM | 2898 | N | PRO | C | 173 | 35.485 | 141.218 | 79.209 | 1.00 | 40.94 | C |
| ATOM | 2899 | CD | PRO | C | 173 | 35.721 | 139.774 | 79.360 | 1.00 | 40.50 | C |
| ATOM | 2900 | CA | PRO | C | 173 | 34.147 | 141.477 | 78.682 | 1.00 | 43.50 | C |
| ATOM | 2901 | CB | PRO | C | 173 | 33.438 | 140.157 | 78.921 | 1.00 | 41.69 | C |
| ATOM | 2902 | CG | PRO | C | 173 | 34.519 | 139.185 | 78.674 | 1.00 | 41.96 | C |
| ATOM | 2903 | C | PRO | C | 173 | 33.442 | 142.637 | 79.390 | 1.00 | 46.36 | C |
| ATOM | 2904 | O | PRO | C | 173 | 33.417 | 142.705 | 80.627 | 1.00 | 46.49 | C |
| ATOM | 2905 | N | ARG | C | 174 | 32.872 | 143.548 | 78.598 | 1.00 | 49.70 | C |
| ATOM | 2906 | CA | ARG | C | 174 | 32.124 | 144.704 | 79.144 | 1.00 | 53.74 | C |
| ATOM | 2907 | CB | ARG | C | 174 | 33.012 | 145.956 | 79.242 | 1.00 | 50.98 | C |
| ATOM | 2908 | CG | ARG | C | 174 | 33.569 | 146.157 | 80.639 | 1.00 | 48.33 | C |
| ATOM | 2909 | CD | ARG | C | 174 | 34.551 | 147.314 | 80.700 | 1.00 | 47.25 | C |
| ATOM | 2910 | NE | ARG | C | 174 | 33.936 | 148.578 | 81.100 | 1.00 | 45.87 | C |
| ATOM | 2911 | CZ | ARG | C | 174 | 34.614 | 149.707 | 81.323 | 1.00 | 44.56 | C |
| ATOM | 2912 | NH1 | ARG | C | 174 | 35.911 | 149.746 | 81.185 | 1.00 | 43.40 | C |
| ATOM | 2913 | NH2 | ARG | C | 174 | 34.003 | 150.804 | 81.723 | 1.00 | 45.12 | C |
| ATOM | 2914 | C | ARG | C | 174 | 30.837 | 145.068 | 78.380 | 1.00 | 56.99 | C |
| ATOM | 2915 | O | ARG | C | 174 | 30.793 | 145.043 | 77.137 | 1.00 | 57.44 | C |
| ATOM | 2916 | N | GLU | C | 175 | 29.807 | 145.428 | 79.143 | 1.00 | 60.80 | C |
| ATOM | 2917 | CA | GLU | C | 175 | 28.520 | 145.798 | 78.566 | 1.00 | 64.68 | C |
| ATOM | 2918 | CB | GLU | C | 175 | 27.482 | 145.909 | 79.677 | 1.00 | 66.10 | C |
| ATOM | 2919 | CG | GLU | C | 175 | 26.086 | 145.700 | 79.183 | 1.00 | 70.38 | C |
| ATOM | 2920 | CD | GLU | C | 175 | 25.839 | 144.311 | 78.618 | 1.00 | 72.64 | C |
| ATOM | 2921 | OE1 | GLU | C | 175 | 24.754 | 144.121 | 78.000 | 1.00 | 74.48 | C |
| ATOM | 2922 | OE2 | GLU | C | 175 | 26.708 | 143.419 | 78.796 | 1.00 | 74.34 | C |
| ATOM | 2923 | C | GLU | C | 175 | 28.665 | 147.120 | 77.819 | 1.00 | 66.00 | C |
| ATOM | 2924 | O | GLU | C | 175 | 29.258 | 148.074 | 78.333 | 1.00 | 65.74 | C |
| ATOM | 2925 | N | PRO | C | 176 | 28.120 | 147.192 | 76.592 | 1.00 | 67.91 | C |
| ATOM | 2926 | CD | PRO | C | 176 | 27.093 | 146.263 | 76.070 | 1.00 | 68.95 | C |
| ATOM | 2927 | CA | PRO | C | 176 | 28.207 | 148.418 | 75.768 | 1.00 | 68.61 | C |
| ATOM | 2928 | CB | PRO | C | 176 | 27.295 | 148.107 | 74.577 | 1.00 | 68.78 | C |
| ATOM | 2929 | CG | PRO | C | 176 | 26.247 | 147.175 | 75.172 | 1.00 | 69.63 | C |
| ATOM | 2930 | C | PRO | C | 176 | 27.751 | 149.623 | 76.583 | 1.00 | 68.77 | C |
| ATOM | 2931 | O | PRO | C | 176 | 26.995 | 149.443 | 77.562 | 1.00 | 68.99 | C |
| ATOM | 2932 | N | ARG | C | 177 | 28.216 | 150.829 | 76.225 | 1.00 | 68.82 | C |
| ATOM | 2933 | CA | ARG | C | 177 | 27.819 | 152.038 | 76.993 | 1.00 | 68.89 | C |
| ATOM | 2934 | CB | ARG | C | 177 | 28.868 | 152.325 | 78.099 | 1.00 | 66.70 | C |
| ATOM | 2935 | CG | ARG | C | 177 | 30.356 | 152.146 | 77.766 | 1.00 | 65.25 | C |
| ATOM | 2936 | CD | ARG | C | 177 | 31.230 | 152.875 | 78.873 | 1.00 | 62.96 | C |
| ATOM | 2937 | NE | ARG | C | 177 | 32.653 | 152.489 | 78.896 | 1.00 | 59.27 | C |
| ATOM | 2938 | CZ | ARG | C | 177 | 33.640 | 153.285 | 79.311 | 1.00 | 57.87 | C |
| ATOM | 2939 | NH1 | ARG | C | 177 | 33.361 | 154.492 | 79.737 | 1.00 | 56.75 | C |
| ATOM | 2940 | NH2 | ARG | C | 177 | 34.913 | 152.896 | 79.273 | 1.00 | 56.60 | C |
| ATOM | 2941 | C | ARG | C | 177 | 27.368 | 153.395 | 76.333 | 1.00 | 69.53 | C |
| ATOM | 2942 | O | ARG | C | 177 | 27.929 | 153.916 | 75.345 | 1.00 | 70.16 | C |
| ATOM | 2943 | N | ALA | C | 178 | 25.383 | 155.781 | 76.002 | 0.00 | 77.76 | C |
| ATOM | 2944 | CA | ALA | C | 178 | 26.177 | 156.720 | 75.219 | 0.00 | 77.74 | C |
| ATOM | 2945 | CB | ALA | C | 178 | 27.608 | 156.758 | 75.743 | 0.00 | 77.77 | C |
| ATOM | 2946 | C | ALA | C | 178 | 26.168 | 156.342 | 73.743 | 0.00 | 77.72 | C |
| ATOM | 2947 | O | ALA | C | 178 | 25.819 | 157.204 | 72.911 | 0.00 | 77.71 | C |
| ATOM | 2948 | OXT | ALA | C | 178 | 26.515 | 155.191 | 73.433 | 1.00 | 77.62 | C |
| ATOM | 2949 | CB | PRO | D | 2 | 45.678 | 107.141 | 70.031 | 1.00 | 32.57 | D |
| ATOM | 2950 | CG | PRO | D | 2 | 44.620 | 107.963 | 69.374 | 1.00 | 33.93 | D |
| ATOM | 2951 | C | PRO | D | 2 | 47.055 | 108.952 | 71.083 | 1.00 | 31.66 | D |
| ATOM | 2952 | O | PRO | D | 2 | 46.807 | 110.088 | 70.797 | 1.00 | 32.83 | D |
| ATOM | 2953 | N | PRO | D | 2 | 44.674 | 108.683 | 71.588 | 1.00 | 34.36 | D |
| ATOM | 2954 | CD | PRO | D | 2 | 43.719 | 108.464 | 70.485 | 1.00 | 33.06 | D |
| ATOM | 2955 | CA | PRO | D | 2 | 45.932 | 107.964 | 71.289 | 1.00 | 33.50 | D |
| ATOM | 2956 | N | ARG | D | 3 | 48.284 | 108.495 | 71.204 | 1.00 | 30.85 | D |
| ATOM | 2957 | CA | ARG | D | 3 | 49.453 | 109.344 | 71.079 | 1.00 | 31.36 | D |
| ATOM | 2958 | CB | ARG | D | 3 | 50.707 | 108.557 | 71.384 | 1.00 | 35.34 | D |
| ATOM | 2959 | CG | ARG | D | 3 | 50.556 | 107.818 | 72.695 | 1.00 | 41.54 | D |
| ATOM | 2960 | CD | ARG | D | 3 | 51.758 | 107.031 | 73.014 | 1.00 | 45.69 | D |
| ATOM | 2961 | NE | ARG | D | 3 | 52.880 | 107.939 | 73.141 | 1.00 | 51.83 | D |
| ATOM | 2962 | CZ | ARG | D | 3 | 54.142 | 107.539 | 73.122 | 1.00 | 53.08 | D |
| ATOM | 2963 | NH1 | ARG | D | 3 | 54.413 | 106.245 | 72.968 | 1.00 | 53.85 | D |
| ATOM | 2964 | NH2 | ARG | D | 3 | 55.113 | 108.427 | 73.285 | 1.00 | 54.27 | D |
| ATOM | 2965 | C | ARG | D | 3 | 49.683 | 110.076 | 69.818 | 1.00 | 30.71 | D |
| ATOM | 2966 | O | ARG | D | 3 | 50.124 | 111.192 | 69.858 | 1.00 | 28.60 | D |
| ATOM | 2967 | N | VAL | D | 4 | 49.399 | 109.448 | 68.689 | 1.00 | 32.15 | D |
| ATOM | 2968 | CA | VAL | D | 4 | 49.580 | 110.083 | 67.406 | 1.00 | 33.15 | D |
| ATOM | 2969 | CB | VAL | D | 4 | 50.708 | 109.446 | 66.710 | 1.00 | 33.58 | D |
| ATOM | 2970 | CG1 | VAL | D | 4 | 52.025 | 109.869 | 67.334 | 1.00 | 31.78 | D |
| ATOM | 2971 | CG2 | VAL | D | 4 | 50.528 | 107.990 | 66.834 | 1.00 | 33.39 | D |
| ATOM | 2972 | C | VAL | D | 4 | 48.369 | 109.976 | 66.497 | 1.00 | 34.72 | D |
| ATOM | 2973 | O | VAL | D | 4 | 47.461 | 109.217 | 66.768 | 1.00 | 34.74 | D |
| ATOM | 2974 | N | VAL | D | 5 | 48.339 | 110.757 | 65.417 | 1.00 | 36.52 | D |
| ATOM | 2975 | CA | VAL | D | 5 | 47.230 | 110.657 | 64.483 | 1.00 | 37.41 | D |
| ATOM | 2976 | CB | VAL | D | 5 | 47.075 | 111.928 | 63.628 | 1.00 | 37.97 | D |
| ATOM | 2977 | CG1 | VAL | D | 5 | 46.728 | 113.085 | 64.494 | 1.00 | 36.67 | D |
| ATOM | 2978 | CG2 | VAL | D | 5 | 48.350 | 112.180 | 62.842 | 1.00 | 37.13 | D |
| ATOM | 2979 | C | VAL | D | 5 | 47.643 | 109.514 | 63.580 | 1.00 | 38.70 | D |
| ATOM | 2980 | O | VAL | D | 5 | 48.799 | 109.126 | 63.571 | 1.00 | 37.88 | D |
| ATOM | 2981 | N | PRO | D | 6 | 46.706 | 108.955 | 62.803 | 1.00 | 40.92 | D |
| ATOM | 2982 | CD | PRO | D | 6 | 45.237 | 109.105 | 62.833 | 1.00 | 40.29 | D |
| ATOM | 2983 | CA | PRO | D | 6 | 47.109 | 107.837 | 61.923 | 1.00 | 41.03 | D |
| ATOM | 2984 | CB | PRO | D | 6 | 45.783 | 107.191 | 61.536 | 1.00 | 39.87 | D |
| ATOM | 2985 | CG | PRO | D | 6 | 44.824 | 108.308 | 61.617 | 1.00 | 40.10 | D |
| ATOM | 2986 | C | PRO | D | 6 | 47.898 | 108.249 | 60.704 | 1.00 | 41.77 | D |
| ATOM | 2987 | O | PRO | D | 6 | 48.466 | 107.398 | 60.049 | 1.00 | 44.57 | D |
| ATOM | 2988 | N | ASP | D | 7 | 47.976 | 109.550 | 60.440 | 1.00 | 40.60 | D |
| ATOM | 2989 | CA | ASP | D | 7 | 48.638 | 110.083 | 59.238 | 1.00 | 40.05 | D |
| ATOM | 2990 | CB | ASP | D | 7 | 47.564 | 110.651 | 58.312 | 1.00 | 39.62 | D |
| ATOM | 2991 | CG | ASP | D | 7 | 46.676 | 111.660 | 59.032 | 1.00 | 40.66 | D |
| ATOM | 2992 | OD1 | ASP | D | 7 | 46.570 | 111.565 | 60.292 | 1.00 | 42.46 | D |
| ATOM | 2993 | OD2 | ASP | D | 7 | 46.093 | 112.547 | 58.374 | 1.00 | 38.89 | D |
| ATOM | 2994 | C | ASP | D | 7 | 49.593 | 111.214 | 59.580 | 1.00 | 39.49 | D |
| ATOM | 2995 | O | ASP | D | 7 | 49.420 | 112.326 | 59.103 | 1.00 | 39.94 | D |
| ATOM | 2996 | N | GLN | D | 8 | 50.580 | 110.926 | 60.415 | 1.00 | 39.45 | D |
| ATOM | 2997 | CA | GLN | D | 8 | 51.548 | 111.911 | 60.880 | 1.00 | 38.36 | D |
| ATOM | 2998 | CB | GLN | D | 8 | 52.649 | 111.233 | 61.718 | 1.00 | 37.26 | D |
| ATOM | 2999 | CG | GLN | D | 8 | 52.151 | 110.300 | 62.752 | 1.00 | 35.10 | D |
| ATOM | 3000 | CD | GLN | D | 8 | 53.234 | 109.783 | 63.669 | 1.00 | 37.73 | D |
| ATOM | 3001 | OE1 | GLN | D | 8 | 53.991 | 110.547 | 64.296 | 1.00 | 34.90 | D |
| ATOM | 3002 | NE2 | GLN | D | 8 | 53.299 | 108.464 | 63.777 | 1.00 | 38.15 | D |
| ATOM | 3003 | C | GLN | D | 8 | 52.205 | 112.678 | 59.762 | 1.00 | 38.74 | D |
| ATOM | 3004 | O | GLN | D | 8 | 52.144 | 113.929 | 59.716 | 1.00 | 38.77 | D |
| ATOM | 3005 | N | ARG | D | 9 | 52.824 | 111.931 | 58.854 | 1.00 | 37.98 | D |
| ATOM | 3006 | CA | ARG | D | 9 | 53.547 | 112.572 | 57.784 | 1.00 | 38.09 | D |
| ATOM | 3007 | CB | ARG | D | 9 | 54.110 | 111.557 | 56.829 | 1.00 | 37.98 | D |
| ATOM | 3008 | CG | ARG | D | 9 | 54.986 | 112.223 | 55.884 | 1.00 | 39.19 | D |
| ATOM | 3009 | CD | ARG | D | 9 | 55.994 | 113.008 | 56.640 | 1.00 | 42.50 | D |
| ATOM | 3010 | NE | ARG | D | 9 | 56.794 | 113.779 | 55.707 | 1.00 | 46.41 | D |
| ATOM | 3011 | CZ | ARG | D | 9 | 56.440 | 114.974 | 55.248 | 1.00 | 49.97 | D |
| ATOM | 3012 | NH1 | ARG | D | 9 | 55.289 | 115.542 | 55.660 | 1.00 | 49.87 | D |
| ATOM | 3013 | NH2 | ARG | D | 9 | 57.220 | 115.592 | 54.357 | 1.00 | 50.87 | D |
| ATOM | 3014 | C | ARG | D | 9 | 52.692 | 113.527 | 57.023 | 1.00 | 37.45 | D |
| ATOM | 3015 | O | ARG | D | 9 | 53.065 | 114.655 | 56.701 | 1.00 | 38.02 | D |
| ATOM | 3016 | N | SER | D | 10 | 51.524 | 113.060 | 56.706 | 1.00 | 38.42 | D |
| ATOM | 3017 | CA | SER | D | 10 | 50.646 | 113.921 | 55.981 | 1.00 | 40.13 | D |
| ATOM | 3018 | CB | SER | D | 10 | 49.394 | 113.170 | 55.587 | 1.00 | 39.84 | D |
| ATOM | 3019 | OG | SER | D | 10 | 48.566 | 114.005 | 54.793 | 1.00 | 38.78 | D |
| ATOM | 3020 | C | SER | D | 10 | 50.268 | 115.191 | 56.792 | 1.00 | 41.58 | D |
| ATOM | 3021 | O | SER | D | 10 | 50.246 | 116.309 | 56.254 | 1.00 | 40.30 | D |
| ATOM | 3022 | N | LYS | D | 11 | 49.949 | 115.013 | 58.072 | 1.00 | 42.37 | D |
| ATOM | 3023 | CA | LYS | D | 11 | 49.622 | 116.131 | 58.931 | 1.00 | 43.42 | D |
| ATOM | 3024 | CB | LYS | D | 11 | 49.422 | 115.646 | 60.348 | 1.00 | 43.78 | D |
| ATOM | 3025 | CG | LYS | D | 11 | 48.531 | 116.590 | 61.068 | 1.00 | 46.63 | D |
| ATOM | 3026 | CD | LYS | D | 11 | 48.027 | 116.126 | 62.439 | 1.00 | 46.71 | D |
| ATOM | 3027 | CE | LYS | D | 11 | 46.966 | 117.117 | 62.875 | 1.00 | 46.44 | D |
| ATOM | 3028 | NZ | LYS | D | 11 | 46.933 | 117.216 | 64.328 | 1.00 | 48.67 | D |
| ATOM | 3029 | C | LYS | D | 11 | 50.755 | 117.149 | 58.906 | 1.00 | 44.23 | D |
| ATOM | 3030 | O | LYS | D | 11 | 50.547 | 118.339 | 58.650 | 1.00 | 44.30 | D |
| ATOM | 3031 | N | PHE | D | 12 | 51.968 | 116.674 | 59.158 | 1.00 | 45.11 | D |
| ATOM | 3032 | CA | PHE | D | 12 | 53.128 | 117.541 | 59.148 | 1.00 | 46.21 | D |
| ATOM | 3033 | CB | PHE | D | 12 | 54.387 | 116.741 | 59.410 | 1.00 | 45.34 | D |
| ATOM | 3034 | CG | PHE | D | 12 | 55.593 | 117.590 | 59.645 | 1.00 | 45.34 | D |
| ATOM | 3035 | CD1 | PHE | D | 12 | 55.760 | 118.272 | 60.847 | 1.00 | 44.60 | D |
| ATOM | 3036 | CD2 | PHE | D | 12 | 56.564 | 117.711 | 58.664 | 1.00 | 43.80 | D |
| ATOM | 3037 | CE1 | PHE | D | 12 | 56.869 | 119.055 | 61.049 | 1.00 | 45.98 | D |
| ATOM | 3038 | CE2 | PHE | D | 12 | 57.678 | 118.493 | 58.858 | 1.00 | 43.63 | D |
| ATOM | 3039 | CZ | PHE | D | 12 | 57.847 | 119.169 | 60.032 | 1.00 | 45.55 | D |
| ATOM | 3040 | C | PHE | D | 12 | 53.307 | 118.301 | 57.830 | 1.00 | 47.76 | D |
| ATOM | 3041 | O | PHE | D | 12 | 53.826 | 119.420 | 57.830 | 1.00 | 48.66 | D |
| ATOM | 3042 | N | GLU | D | 13 | 52.885 | 117.724 | 56.711 | 1.00 | 48.82 | D |
| ATOM | 3043 | CA | GLU | D | 13 | 53.052 | 118.408 | 55.432 | 1.00 | 50.97 | D |
| ATOM | 3044 | CB | GLU | D | 13 | 53.107 | 117.412 | 54.259 | 1.00 | 53.60 | D |
| ATOM | 3045 | CG | GLU | D | 13 | 54.497 | 117.053 | 53.664 | 1.00 | 56.92 | D |
| ATOM | 3046 | CD | GLU | D | 13 | 55.386 | 118.244 | 53.284 | 1.00 | 59.65 | D |
| ATOM | 3047 | OE1 | GLU | D | 13 | 56.067 | 118.782 | 54.213 | 1.00 | 60.58 | D |
| ATOM | 3048 | OE2 | GLU | D | 13 | 55.408 | 118.618 | 52.069 | 1.00 | 59.30 | D |
| ATOM | 3049 | C | GLU | D | 13 | 51.950 | 119.364 | 55.079 | 1.00 | 50.36 | D |
| ATOM | 3050 | O | GLU | D | 13 | 52.180 | 120.388 | 54.464 | 1.00 | 50.65 | D |
| ATOM | 3051 | N | ASN | D | 14 | 50.736 | 119.015 | 55.454 | 1.00 | 50.28 | D |
| ATOM | 3052 | CA | ASN | D | 14 | 49.609 | 119.801 | 55.030 | 1.00 | 50.52 | D |
| ATOM | 3053 | CB | ASN | D | 14 | 48.497 | 118.872 | 54.529 | 1.00 | 51.45 | D |
| ATOM | 3054 | CG | ASN | D | 14 | 48.951 | 117.998 | 53.347 | 1.00 | 52.34 | D |
| ATOM | 3055 | OD1 | ASN | D | 14 | 49.612 | 118.485 | 52.416 | 1.00 | 51.01 | D |
| ATOM | 3056 | ND2 | ASN | D | 14 | 48.592 | 116.707 | 53.385 | 1.00 | 52.12 | D |
| ATOM | 3057 | C | ASN | D | 14 | 49.047 | 120.800 | 55.970 | 1.00 | 50.85 | D |
| ATOM | 3058 | O | ASN | D | 14 | 48.393 | 121.744 | 55.543 | 1.00 | 51.13 | D |
| ATOM | 3059 | N | GLU | D | 15 | 49.274 | 120.635 | 57.252 | 1.00 | 50.98 | D |
| ATOM | 3060 | CA | GLU | D | 15 | 48.738 | 121.658 | 58.115 | 1.00 | 51.60 | D |
| ATOM | 3061 | CB | GLU | D | 15 | 48.513 | 121.145 | 59.495 | 1.00 | 53.94 | D |
| ATOM | 3062 | CG | GLU | D | 15 | 47.558 | 120.026 | 59.536 | 1.00 | 57.72 | D |
| ATOM | 3063 | CD | GLU | D | 15 | 46.845 | 119.975 | 60.838 | 1.00 | 59.75 | D |
| ATOM | 3064 | OE1 | GLU | D | 15 | 47.504 | 119.987 | 61.915 | 1.00 | 62.07 | D |
| ATOM | 3065 | OE2 | GLU | D | 15 | 45.600 | 119.930 | 60.783 | 1.00 | 62.42 | D |
| ATOM | 3066 | C | GLU | D | 15 | 49.609 | 122.873 | 58.199 | 1.00 | 50.81 | D |
| ATOM | 3067 | O | GLU | D | 15 | 50.809 | 122.796 | 58.370 | 1.00 | 50.27 | D |
| ATOM | 3068 | N | GLU | D | 16 | 48.970 | 124.013 | 58.079 | 1.00 | 51.06 | D |
| ATOM | 3069 | CA | GLU | D | 16 | 49.660 | 125.274 | 58.125 | 1.00 | 51.27 | D |
| ATOM | 3070 | CB | GLU | D | 16 | 48.630 | 126.372 | 58.001 | 1.00 | 54.24 | D |
| ATOM | 3071 | CG | GLU | D | 16 | 49.166 | 127.742 | 57.664 | 1.00 | 61.05 | D |
| ATOM | 3072 | CD | GLU | D | 16 | 48.038 | 128.758 | 57.551 | 1.00 | 63.95 | D |
| ATOM | 3073 | OE1 | GLU | D | 16 | 47.209 | 128.828 | 58.491 | 1.00 | 66.45 | D |
| ATOM | 3074 | OE2 | GLU | D | 16 | 47.971 | 129.481 | 56.532 | 1.00 | 65.95 | D |
| ATOM | 3075 | C | GLU | D | 16 | 50.436 | 125.379 | 59.427 | 1.00 | 49.59 | D |
| ATOM | 3076 | O | GLU | D | 16 | 51.558 | 125.892 | 59.446 | 1.00 | 48.91 | D |
| ATOM | 3077 | N | PHE | D | 17 | 49.852 | 124.865 | 60.508 | 1.00 | 47.34 | D |
| ATOM | 3078 | CA | PHE | D | 17 | 50.536 | 124.904 | 61.796 | 1.00 | 44.93 | D |
| ATOM | 3079 | CB | PHE | D | 17 | 49.841 | 124.027 | 62.837 | 1.00 | 41.09 | D |
| ATOM | 3080 | CG | PHE | D | 17 | 50.556 | 123.965 | 64.161 | 1.00 | 37.49 | D |
| ATOM | 3081 | CD1 | PHE | D | 17 | 50.618 | 125.077 | 64.987 | 1.00 | 34.81 | D |
| ATOM | 3082 | CD2 | PHE | D | 17 | 51.246 | 122.809 | 64.543 | 1.00 | 36.15 | D |
| ATOM | 3083 | CE1 | PHE | D | 17 | 51.367 | 125.046 | 66.175 | 1.00 | 35.31 | D |
| ATOM | 3084 | CE2 | PHE | D | 17 | 51.995 | 122.770 | 65.724 | 1.00 | 36.03 | D |
| ATOM | 3085 | CZ | PHE | D | 17 | 52.065 | 123.888 | 66.552 | 1.00 | 34.89 | D |
| ATOM | 3086 | C | PHE | D | 17 | 51.970 | 124.426 | 61.671 | 1.00 | 46.16 | D |
| ATOM | 3087 | O | PHE | D | 17 | 52.894 | 125.103 | 62.142 | 1.00 | 46.89 | D |
| ATOM | 3088 | N | PHE | D | 18 | 52.166 | 123.251 | 61.071 | 1.00 | 46.74 | D |
| ATOM | 3089 | CA | PHE | D | 18 | 53.516 | 122.714 | 60.918 | 1.00 | 46.92 | D |
| ATOM | 3090 | CB | PHE | D | 18 | 53.462 | 121.218 | 60.652 | 1.00 | 45.35 | D |
| ATOM | 3091 | CG | PHE | D | 18 | 52.931 | 120.431 | 61.810 | 1.00 | 44.22 | D |
| ATOM | 3092 | CD1 | PHE | D | 18 | 51.652 | 119.906 | 61.783 | 1.00 | 41.94 | D |
| ATOM | 3093 | CD2 | PHE | D | 18 | 53.734 | 120.222 | 62.934 | 1.00 | 44.05 | D |
| ATOM | 3094 | CE1 | PHE | D | 18 | 51.169 | 119.181 | 62.840 | 1.00 | 43.79 | D |
| ATOM | 3095 | CE2 | PHE | D | 18 | 53.264 | 119.496 | 64.013 | 1.00 | 45.05 | D |
| ATOM | 3096 | CZ | PHE | D | 18 | 51.970 | 118.965 | 63.974 | 1.00 | 44.94 | D |
| ATOM | 3097 | C | PHE | D | 18 | 54.311 | 123.444 | 59.840 | 1.00 | 47.62 | D |
| ATOM | 3098 | O | PHE | D | 18 | 55.450 | 123.787 | 60.073 | 1.00 | 46.32 | D |
| ATOM | 3099 | N | ARG | D | 19 | 53.701 | 123.712 | 58.683 | 1.00 | 49.64 | D |
| ATOM | 3100 | CA | ARG | D | 19 | 54.368 | 124.437 | 57.584 | 1.00 | 51.75 | D |
| ATOM | 3101 | CB | ARG | D | 19 | 53.340 | 124.793 | 56.501 | 1.00 | 54.74 | D |
| ATOM | 3102 | CG | ARG | D | 19 | 52.643 | 123.567 | 55.882 | 1.00 | 59.49 | D |
| ATOM | 3103 | CD | ARG | D | 19 | 51.764 | 123.920 | 54.656 | 1.00 | 62.91 | D |
| ATOM | 3104 | NE | ARG | D | 19 | 51.977 | 122.936 | 53.599 | 1.00 | 67.30 | D |
| ATOM | 3105 | CZ | ARG | D | 19 | 53.015 | 122.950 | 52.757 | 1.00 | 69.38 | D |
| ATOM | 3106 | NH1 | ARG | D | 19 | 53.922 | 123.918 | 52.837 | 1.00 | 69.45 | D |
| ATOM | 3107 | NH2 | ARG | D | 19 | 53.192 | 121.963 | 51.878 | 1.00 | 69.91 | D |
| ATOM | 3108 | C | ARG | D | 19 | 55.065 | 125.727 | 58.081 | 1.00 | 51.25 | D |
| ATOM | 3109 | O | ARG | D | 19 | 56.236 | 126.004 | 57.792 | 1.00 | 50.41 | D |
| ATOM | 3110 | N | LYS | D | 20 | 54.343 | 126.501 | 58.866 | 1.00 | 50.86 | D |
| ATOM | 3111 | CA | LYS | D | 20 | 54.896 | 127.721 | 59.387 | 1.00 | 50.66 | D |
| ATOM | 3112 | CB | LYS | D | 20 | 53.822 | 128.476 | 60.145 | 1.00 | 52.19 | D |
| ATOM | 3113 | CG | LYS | D | 20 | 52.883 | 129.205 | 59.264 | 1.00 | 55.08 | D |
| ATOM | 3114 | CD | LYS | D | 20 | 52.071 | 130.211 | 60.033 | 1.00 | 58.75 | D |
| ATOM | 3115 | CE | LYS | D | 20 | 51.097 | 130.934 | 59.092 | 1.00 | 61.52 | D |
| ATOM | 3116 | NZ | LYS | D | 20 | 49.747 | 131.250 | 59.718 | 1.00 | 63.73 | D |
| ATOM | 3117 | C | LYS | D | 20 | 56.067 | 127.504 | 60.307 | 1.00 | 50.60 | D |
| ATOM | 3118 | O | LYS | D | 20 | 56.778 | 128.431 | 60.653 | 1.00 | 51.50 | D |
| ATOM | 3119 | N | LEU | D | 21 | 56.276 | 126.283 | 60.749 | 1.00 | 51.13 | D |
| ATOM | 3120 | CA | LEU | D | 21 | 57.369 | 126.032 | 61.676 | 1.00 | 51.75 | D |
| ATOM | 3121 | CB | LEU | D | 21 | 56.818 | 125.401 | 62.971 | 1.00 | 50.02 | D |
| ATOM | 3122 | CG | LEU | D | 21 | 56.302 | 126.250 | 64.149 | 1.00 | 49.40 | D |
| ATOM | 3123 | CD1 | LEU | D | 21 | 56.283 | 127.713 | 63.808 | 1.00 | 48.92 | D |
| ATOM | 3124 | CD2 | LEU | D | 21 | 54.897 | 125.777 | 64.511 | 1.00 | 49.81 | D |
| ATOM | 3125 | C | LEU | D | 21 | 58.455 | 125.127 | 61.094 | 1.00 | 53.18 | D |
| ATOM | 3126 | O | LEU | D | 21 | 59.437 | 124.829 | 61.790 | 1.00 | 53.01 | D |
| ATOM | 3127 | N | SER | D | 22 | 58.312 | 124.719 | 59.825 | 1.00 | 54.02 | D |
| ATOM | 3128 | CA | SER | D | 22 | 59.261 | 123.777 | 59.273 | 1.00 | 55.58 | D |
| ATOM | 3129 | CB | SER | D | 22 | 58.580 | 122.836 | 58.303 | 1.00 | 54.51 | D |
| ATOM | 3130 | OG | SER | D | 22 | 57.794 | 123.567 | 57.419 | 1.00 | 56.53 | D |
| ATOM | 3131 | C | SER | D | 22 | 60.491 | 124.355 | 58.670 | 1.00 | 57.18 | D |
| ATOM | 3132 | O | SER | D | 22 | 61.443 | 123.635 | 58.496 | 1.00 | 57.69 | D |
| ATOM | 3133 | N | ARG | D | 23 | 60.474 | 125.645 | 58.363 | 1.00 | 59.62 | D |
| ATOM | 3134 | CA | ARG | D | 23 | 61.626 | 126.374 | 57.814 | 1.00 | 62.09 | D |
| ATOM | 3135 | CB | ARG | D | 23 | 61.186 | 127.439 | 56.790 | 1.00 | 65.46 | D |
| ATOM | 3136 | CG | ARG | D | 23 | 60.962 | 127.009 | 55.328 | 1.00 | 71.15 | D |
| ATOM | 3137 | CD | ARG | D | 23 | 59.713 | 126.122 | 55.078 | 1.00 | 75.91 | D |
| ATOM | 3138 | NE | ARG | D | 23 | 59.472 | 125.941 | 53.635 | 1.00 | 79.79 | D |
| ATOM | 3139 | CZ | ARG | D | 23 | 58.774 | 124.941 | 53.087 | 1.00 | 81.32 | D |
| ATOM | 3140 | NH1 | ARG | D | 23 | 58.224 | 123.987 | 53.854 | 1.00 | 82.05 | D |
| ATOM | 3141 | NH2 | ARG | D | 23 | 58.618 | 124.896 | 51.758 | 1.00 | 80.91 | D |
| ATOM | 3142 | C | ARG | D | 23 | 62.168 | 127.127 | 59.032 | 1.00 | 62.31 | D |
| ATOM | 3143 | O | ARG | D | 23 | 61.381 | 127.518 | 59.892 | 1.00 | 63.40 | D |
| ATOM | 3144 | N | GLU | D | 24 | 63.474 | 127.361 | 59.114 | 1.00 | 61.88 | D |
| ATOM | 3145 | CA | GLU | D | 24 | 64.034 | 128.110 | 60.246 | 1.00 | 61.51 | D |
| ATOM | 3146 | CB | GLU | D | 24 | 65.525 | 128.267 | 60.074 | 1.00 | 63.62 | D |
| ATOM | 3147 | CG | GLU | D | 24 | 66.256 | 126.976 | 60.322 | 1.00 | 67.85 | D |
| ATOM | 3148 | CD | GLU | D | 24 | 67.644 | 126.992 | 59.737 | 1.00 | 70.09 | D |
| ATOM | 3149 | OE1 | GLU | D | 24 | 68.357 | 128.002 | 59.971 | 1.00 | 70.97 | D |
| ATOM | 3150 | OE2 | GLU | D | 24 | 68.005 | 125.999 | 59.046 | 1.00 | 71.36 | D |
| ATOM | 3151 | C | GLU | D | 24 | 63.437 | 129.492 | 60.470 | 1.00 | 59.59 | D |
| ATOM | 3152 | O | GLU | D | 24 | 63.131 | 130.215 | 59.524 | 1.00 | 59.25 | D |
| ATOM | 3153 | N | CYS | D | 25 | 63.270 | 129.861 | 61.730 | 1.00 | 57.08 | D |
| ATOM | 3154 | CA | CYS | D | 25 | 62.722 | 131.168 | 62.014 | 1.00 | 55.92 | D |
| ATOM | 3155 | CB | CYS | D | 25 | 61.180 | 131.214 | 61.842 | 1.00 | 56.43 | D |
| ATOM | 3156 | SG | CYS | D | 25 | 60.264 | 129.809 | 62.419 | 1.00 | 55.81 | D |
| ATOM | 3157 | C | CYS | D | 25 | 63.113 | 131.648 | 63.368 | 1.00 | 54.32 | D |
| ATOM | 3158 | O | CYS | D | 25 | 63.718 | 130.900 | 64.147 | 1.00 | 53.13 | D |
| ATOM | 3159 | N | GLU | D | 26 | 62.778 | 132.909 | 63.626 | 1.00 | 52.98 | D |
| ATOM | 3160 | CA | GLU | D | 26 | 63.112 | 133.565 | 64.878 | 1.00 | 52.95 | D |
| ATOM | 3161 | CB | GLU | D | 26 | 62.681 | 135.029 | 64.794 | 1.00 | 53.72 | D |
| ATOM | 3162 | CG | GLU | D | 26 | 63.102 | 135.868 | 65.978 | 1.00 | 56.93 | D |
| ATOM | 3163 | CD | GLU | D | 26 | 62.696 | 137.324 | 65.811 | 1.00 | 59.66 | D |
| ATOM | 3164 | OE1 | GLU | D | 26 | 62.849 | 138.106 | 66.784 | 1.00 | 59.27 | D |
| ATOM | 3165 | OE2 | GLU | D | 26 | 62.218 | 137.691 | 64.693 | 1.00 | 61.89 | D |
| ATOM | 3166 | C | GLU | D | 26 | 62.525 | 132.903 | 66.125 | 1.00 | 51.88 | D |
| ATOM | 3167 | O | GLU | D | 26 | 61.319 | 132.799 | 66.287 | 1.00 | 52.32 | D |
| ATOM | 3168 | N | ILE | D | 27 | 63.388 | 132.425 | 66.994 | 1.00 | 50.87 | D |
| ATOM | 3169 | CA | ILE | D | 27 | 62.925 | 131.820 | 68.225 | 1.00 | 51.36 | D |
| ATOM | 3170 | CB | ILE | D | 27 | 63.403 | 130.402 | 68.369 | 1.00 | 50.81 | D |
| ATOM | 3171 | CG2 | ILE | D | 27 | 62.660 | 129.538 | 67.392 | 1.00 | 50.25 | D |
| ATOM | 3172 | CG1 | ILE | D | 27 | 64.909 | 130.347 | 68.144 | 1.00 | 50.66 | D |
| ATOM | 3173 | CD1 | ILE | D | 27 | 65.581 | 129.166 | 68.779 | 1.00 | 51.45 | D |
| ATOM | 3174 | C | ILE | D | 27 | 63.476 | 132.675 | 69.352 | 1.00 | 51.85 | D |
| ATOM | 3175 | O | ILE | D | 27 | 64.217 | 133.614 | 69.107 | 1.00 | 53.10 | D |
| ATOM | 3176 | N | LYS | D | 28 | 63.121 | 132.376 | 70.586 | 1.00 | 51.70 | D |
| ATOM | 3177 | CA | LYS | D | 28 | 63.584 | 133.205 | 71.669 | 1.00 | 51.81 | D |
| ATOM | 3178 | CB | LYS | D | 28 | 62.878 | 134.557 | 71.547 | 1.00 | 51.60 | D |
| ATOM | 3179 | CG | LYS | D | 28 | 63.201 | 135.519 | 72.626 | 1.00 | 53.02 | D |
| ATOM | 3180 | CD | LYS | D | 28 | 62.571 | 136.888 | 72.369 | 1.00 | 54.12 | D |
| ATOM | 3181 | CE | LYS | D | 28 | 63.094 | 137.568 | 71.121 | 1.00 | 52.86 | D |
| ATOM | 3182 | NZ | LYS | D | 28 | 63.036 | 139.051 | 71.314 | 1.00 | 53.52 | D |
| ATOM | 3183 | C | LYS | D | 28 | 63.289 | 132.539 | 73.028 | 1.00 | 52.51 | D |
| ATOM | 3184 | O | LYS | D | 28 | 62.221 | 131.910 | 73.216 | 1.00 | 52.46 | D |
| ATOM | 3185 | N | TYR | D | 29 | 64.248 | 132.652 | 73.954 | 1.00 | 51.77 | D |
| ATOM | 3186 | CA | TYR | D | 29 | 64.114 | 132.098 | 75.300 | 1.00 | 50.80 | D |
| ATOM | 3187 | CB | TYR | D | 29 | 65.424 | 132.257 | 76.066 | 1.00 | 49.96 | D |
| ATOM | 3188 | CG | TYR | D | 29 | 65.364 | 131.807 | 77.488 | 1.00 | 48.55 | D |
| ATOM | 3189 | CD1 | TYR | D | 29 | 65.043 | 130.500 | 77.802 | 1.00 | 49.31 | D |
| ATOM | 3190 | CE1 | TYR | D | 29 | 64.999 | 130.067 | 79.120 | 1.00 | 49.17 | D |
| ATOM | 3191 | CD2 | TYR | D | 29 | 65.637 | 132.686 | 78.524 | 1.00 | 49.27 | D |
| ATOM | 3192 | CE2 | TYR | D | 29 | 65.590 | 132.273 | 79.860 | 1.00 | 48.43 | D |
| ATOM | 3193 | CZ | TYR | D | 29 | 65.269 | 130.958 | 80.149 | 1.00 | 48.63 | D |
| ATOM | 3194 | OH | TYR | D | 29 | 65.191 | 130.514 | 81.460 | 1.00 | 49.15 | D |
| ATOM | 3195 | C | TYR | D | 29 | 63.034 | 132.906 | 75.993 | 1.00 | 51.04 | D |
| ATOM | 3196 | O | TYR | D | 29 | 63.059 | 134.122 | 75.922 | 1.00 | 51.67 | D |
| ATOM | 3197 | N | THR | D | 30 | 62.105 | 132.241 | 76.663 | 1.00 | 50.51 | D |
| ATOM | 3198 | CA | THR | D | 30 | 61.000 | 132.915 | 77.332 | 1.00 | 49.97 | D |
| ATOM | 3199 | CB | THR | D | 30 | 59.637 | 132.346 | 76.852 | 1.00 | 48.64 | D |
| ATOM | 3200 | OG1 | THR | D | 30 | 59.515 | 130.964 | 77.252 | 1.00 | 47.80 | D |
| ATOM | 3201 | CG2 | THR | D | 30 | 59.527 | 132.445 | 75.366 | 1.00 | 46.51 | D |
| ATOM | 3202 | C | THR | D | 30 | 61.039 | 132.742 | 78.855 | 1.00 | 50.98 | D |
| ATOM | 3203 | O | THR | D | 30 | 60.154 | 133.240 | 79.565 | 1.00 | 50.01 | D |
| ATOM | 3204 | N | GLY | D | 31 | 62.041 | 132.010 | 79.344 | 1.00 | 51.66 | D |
| ATOM | 3205 | CA | GLY | D | 31 | 62.155 | 131.746 | 80.770 | 1.00 | 52.79 | D |
| ATOM | 3206 | C | GLY | D | 31 | 62.569 | 132.956 | 81.592 | 1.00 | 54.45 | D |
| ATOM | 3207 | O | GLY | D | 31 | 63.305 | 133.827 | 81.111 | 1.00 | 54.26 | D |
| ATOM | 3208 | N | PHE | D | 32 | 62.110 | 132.990 | 82.843 | 1.00 | 56.20 | D |
| ATOM | 3209 | CA | PHE | D | 32 | 62.367 | 134.090 | 83.778 | 1.00 | 58.64 | D |
| ATOM | 3210 | CB | PHE | D | 32 | 63.709 | 133.894 | 84.463 | 1.00 | 58.34 | D |
| ATOM | 3211 | CG | PHE | D | 32 | 63.864 | 132.551 | 85.106 | 1.00 | 57.16 | D |
| ATOM | 3212 | CD1 | PHE | D | 32 | 64.209 | 131.443 | 84.337 | 1.00 | 56.91 | D |
| ATOM | 3213 | CD2 | PHE | D | 32 | 63.650 | 132.395 | 86.472 | 1.00 | 56.18 | D |
| ATOM | 3214 | CE1 | PHE | D | 32 | 64.343 | 130.183 | 84.915 | 1.00 | 57.34 | D |
| ATOM | 3215 | CE2 | PHE | D | 32 | 63.774 | 131.147 | 87.076 | 1.00 | 55.92 | D |
| ATOM | 3216 | CZ | PHE | D | 32 | 64.124 | 130.028 | 86.304 | 1.00 | 56.72 | D |
| ATOM | 3217 | C | PHE | D | 32 | 62.304 | 135.477 | 83.146 | 1.00 | 60.47 | D |
| ATOM | 3218 | O | PHE | D | 32 | 63.197 | 136.297 | 83.308 | 1.00 | 60.88 | D |
| ATOM | 3219 | N | ARG | D | 33 | 61.217 | 135.736 | 82.443 | 1.00 | 63.08 | D |
| ATOM | 3220 | CA | ARG | D | 33 | 61.039 | 136.998 | 81.774 | 1.00 | 66.21 | D |
| ATOM | 3221 | CB | ARG | D | 33 | 59.692 | 136.995 | 81.050 | 1.00 | 67.35 | D |
| ATOM | 3222 | CG | ARG | D | 33 | 59.750 | 136.356 | 79.671 | 1.00 | 70.67 | D |
| ATOM | 3223 | CD | ARG | D | 33 | 58.382 | 136.451 | 78.914 | 1.00 | 73.89 | D |
| ATOM | 3224 | NE | ARG | D | 33 | 58.076 | 137.789 | 78.353 | 1.00 | 75.50 | D |
| ATOM | 3225 | CZ | ARG | D | 33 | 58.703 | 138.331 | 77.306 | 1.00 | 75.61 | D |
| ATOM | 3226 | NH1 | ARG | D | 33 | 59.689 | 137.663 | 76.689 | 1.00 | 75.01 | D |
| ATOM | 3227 | NH2 | ARG | D | 33 | 58.321 | 139.531 | 76.861 | 1.00 | 75.78 | D |
| ATOM | 3228 | C | ARG | D | 33 | 61.144 | 138.238 | 82.654 | 1.00 | 67.76 | D |
| ATOM | 3229 | O | ARG | D | 33 | 61.514 | 139.300 | 82.179 | 1.00 | 68.03 | D |
| ATOM | 3230 | N | ASP | D | 34 | 60.823 | 138.121 | 83.928 | 1.00 | 69.50 | D |
| ATOM | 3231 | CA | ASP | D | 34 | 60.846 | 139.290 | 84.800 | 1.00 | 72.39 | D |
| ATOM | 3232 | CB | ASP | D | 34 | 59.798 | 139.118 | 85.905 | 1.00 | 73.15 | D |
| ATOM | 3233 | CG | ASP | D | 34 | 59.973 | 137.822 | 86.689 | 1.00 | 74.40 | D |
| ATOM | 3234 | OD1 | ASP | D | 34 | 60.597 | 136.860 | 86.174 | 1.00 | 75.03 | D |
| ATOM | 3235 | OD2 | ASP | D | 34 | 59.460 | 137.761 | 87.827 | 1.00 | 74.75 | D |
| ATOM | 3236 | C | ASP | D | 34 | 62.203 | 139.673 | 85.420 | 1.00 | 73.42 | D |
| ATOM | 3237 | O | ASP | D | 34 | 62.377 | 140.811 | 85.882 | 1.00 | 73.86 | D |
| ATOM | 3238 | N | ARG | D | 35 | 63.155 | 138.741 | 85.408 | 1.00 | 73.53 | D |
| ATOM | 3239 | CA | ARG | D | 35 | 64.481 | 138.968 | 85.974 | 1.00 | 74.06 | D |
| ATOM | 3240 | CB | ARG | D | 35 | 65.137 | 137.632 | 86.309 | 1.00 | 74.21 | D |
| ATOM | 3241 | CG | ARG | D | 35 | 64.661 | 137.041 | 87.610 | 1.00 | 75.32 | D |
| ATOM | 3242 | CD | ARG | D | 35 | 63.147 | 137.150 | 87.712 | 1.00 | 77.19 | D |
| ATOM | 3243 | NE | ARG | D | 35 | 62.576 | 136.586 | 88.935 | 1.00 | 77.72 | D |
| ATOM | 3244 | CZ | ARG | D | 35 | 62.989 | 135.465 | 89.512 | 1.00 | 78.37 | D |
| ATOM | 3245 | NH1 | ARG | D | 35 | 64.004 | 134.777 | 88.995 | 1.00 | 78.21 | D |
| ATOM | 3246 | NH2 | ARG | D | 35 | 62.346 | 135.011 | 90.580 | 1.00 | 78.75 | D |
| ATOM | 3247 | C | ARG | D | 35 | 65.403 | 139.758 | 85.060 | 1.00 | 74.25 | D |
| ATOM | 3248 | O | ARG | D | 35 | 65.181 | 139.810 | 83.860 | 1.00 | 73.92 | D |
| ATOM | 3249 | N | PRO | D | 36 | 66.469 | 140.378 | 85.629 | 1.00 | 74.71 | D |
| ATOM | 3250 | CD | PRO | D | 36 | 66.887 | 140.335 | 87.046 | 1.00 | 74.32 | D |
| ATOM | 3251 | CA | PRO | D | 36 | 67.428 | 141.170 | 84.841 | 1.00 | 74.16 | D |
| ATOM | 3252 | CB | PRO | D | 36 | 68.474 | 141.591 | 85.876 | 1.00 | 73.55 | D |
| ATOM | 3253 | CG | PRO | D | 36 | 67.735 | 141.583 | 87.152 | 1.00 | 73.93 | D |
| ATOM | 3254 | C | PRO | D | 36 | 68.036 | 140.289 | 83.762 | 1.00 | 74.00 | D |
| ATOM | 3255 | O | PRO | D | 36 | 68.199 | 139.074 | 83.952 | 1.00 | 73.57 | D |
| ATOM | 3256 | N | HIS | D | 37 | 68.396 | 140.900 | 82.642 | 1.00 | 73.90 | D |
| ATOM | 3257 | CA | HIS | D | 37 | 68.961 | 140.119 | 81.559 | 1.00 | 73.74 | D |
| ATOM | 3258 | CB | HIS | D | 37 | 69.386 | 141.017 | 80.411 | 1.00 | 73.90 | D |
| ATOM | 3259 | CG | HIS | D | 37 | 69.909 | 140.260 | 79.225 | 1.00 | 74.60 | D |
| ATOM | 3260 | CD2 | HIS | D | 37 | 71.164 | 140.138 | 78.724 | 1.00 | 74.70 | D |
| ATOM | 3261 | ND1 | HIS | D | 37 | 69.091 | 139.545 | 78.375 | 1.00 | 74.78 | D |
| ATOM | 3262 | CE1 | HIS | D | 37 | 69.818 | 139.025 | 77.400 | 1.00 | 74.71 | D |
| ATOM | 3263 | NE2 | HIS | D | 37 | 71.079 | 139.370 | 77.589 | 1.00 | 74.24 | D |
| ATOM | 3264 | C | HIS | D | 37 | 70.118 | 139.210 | 81.925 | 1.00 | 73.29 | D |
| ATOM | 3265 | O | HIS | D | 37 | 70.152 | 138.060 | 81.488 | 1.00 | 73.88 | D |
| ATOM | 3266 | N | GLU | D | 38 | 71.062 | 139.696 | 82.723 | 1.00 | 73.37 | D |
| ATOM | 3267 | CA | GLU | D | 38 | 72.237 | 138.883 | 83.075 | 1.00 | 73.46 | D |
| ATOM | 3268 | CB | GLU | D | 38 | 73.173 | 139.667 | 83.990 | 1.00 | 75.72 | D |
| ATOM | 3269 | CG | GLU | D | 38 | 73.818 | 140.900 | 83.326 | 1.00 | 78.68 | D |
| ATOM | 3270 | CD | GLU | D | 38 | 72.785 | 141.880 | 82.706 | 1.00 | 80.48 | D |
| ATOM | 3271 | OE1 | GLU | D | 38 | 71.831 | 142.300 | 83.403 | 1.00 | 80.79 | D |
| ATOM | 3272 | OE2 | GLU | D | 38 | 72.927 | 142.241 | 81.512 | 1.00 | 81.66 | D |
| ATOM | 3273 | C | GLU | D | 38 | 71.851 | 137.569 | 83.729 | 1.00 | 72.30 | D |
| ATOM | 3274 | O | GLU | D | 38 | 72.432 | 136.519 | 83.440 | 1.00 | 71.65 | D |
| ATOM | 3275 | N | GLU | D | 39 | 70.852 | 137.629 | 84.600 | 1.00 | 71.02 | D |
| ATOM | 3276 | CA | GLU | D | 39 | 70.373 | 136.438 | 85.273 | 1.00 | 69.39 | D |
| ATOM | 3277 | CB | GLU | D | 39 | 69.313 | 136.815 | 86.315 | 1.00 | 69.29 | D |
| ATOM | 3278 | CG | GLU | D | 39 | 68.970 | 135.686 | 87.286 | 1.00 | 69.82 | D |
| ATOM | 3279 | CD | GLU | D | 39 | 67.874 | 136.076 | 88.261 | 1.00 | 70.37 | D |
| ATOM | 3280 | OE1 | GLU | D | 39 | 67.867 | 137.237 | 88.715 | 1.00 | 70.23 | D |
| ATOM | 3281 | OE2 | GLU | D | 39 | 67.025 | 135.220 | 88.583 | 1.00 | 71.23 | D |
| ATOM | 3282 | C | GLU | D | 39 | 69.761 | 135.505 | 84.221 | 1.00 | 68.82 | D |
| ATOM | 3283 | O | GLU | D | 39 | 70.086 | 134.318 | 84.143 | 1.00 | 68.12 | D |
| ATOM | 3284 | N | ARG | D | 40 | 68.863 | 136.057 | 83.417 | 1.00 | 67.87 | D |
| ATOM | 3285 | CA | ARG | D | 40 | 68.201 | 135.265 | 82.407 | 1.00 | 67.91 | D |
| ATOM | 3286 | CB | ARG | D | 40 | 67.336 | 136.154 | 81.500 | 1.00 | 67.72 | D |
| ATOM | 3287 | CG | ARG | D | 40 | 66.196 | 136.865 | 82.241 | 1.00 | 66.77 | D |
| ATOM | 3288 | CD | ARG | D | 40 | 65.151 | 137.364 | 81.270 | 1.00 | 66.00 | D |
| ATOM | 3289 | NE | ARG | D | 40 | 64.540 | 136.229 | 80.585 | 1.00 | 66.23 | D |
| ATOM | 3290 | CZ | ARG | D | 40 | 64.410 | 136.122 | 79.263 | 1.00 | 65.72 | D |
| ATOM | 3291 | NH1 | ARG | D | 40 | 64.846 | 137.106 | 78.465 | 1.00 | 65.14 | D |
| ATOM | 3292 | NH2 | ARG | D | 40 | 63.873 | 135.022 | 78.748 | 1.00 | 63.97 | D |
| ATOM | 3293 | C | ARG | D | 40 | 69.269 | 134.536 | 81.615 | 1.00 | 67.89 | D |
| ATOM | 3294 | O | ARG | D | 40 | 69.064 | 133.393 | 81.162 | 1.00 | 67.97 | D |
| ATOM | 3295 | N | GLN | D | 41 | 70.414 | 135.184 | 81.445 | 1.00 | 66.86 | D |
| ATOM | 3296 | CA | GLN | D | 41 | 71.491 | 134.523 | 80.729 | 1.00 | 66.92 | D |
| ATOM | 3297 | CB | GLN | D | 41 | 72.635 | 135.495 | 80.514 | 1.00 | 66.33 | D |
| ATOM | 3298 | CG | GLN | D | 41 | 72.579 | 136.216 | 79.216 | 1.00 | 66.20 | D |
| ATOM | 3299 | CD | GLN | D | 41 | 73.452 | 137.408 | 79.269 | 1.00 | 66.03 | D |
| ATOM | 3300 | OE1 | GLN | D | 41 | 73.929 | 137.900 | 78.252 | 1.00 | 66.02 | D |
| ATOM | 3301 | NE2 | GLN | D | 41 | 73.666 | 137.906 | 80.482 | 1.00 | 66.32 | D |
| ATOM | 3302 | C | GLN | D | 41 | 71.994 | 133.277 | 81.493 | 1.00 | 66.04 | D |
| ATOM | 3303 | O | GLN | D | 41 | 72.199 | 132.198 | 80.916 | 1.00 | 65.90 | D |
| ATOM | 3304 | N | ALA | D | 42 | 72.195 | 133.442 | 82.790 | 1.00 | 65.46 | D |
| ATOM | 3305 | CA | ALA | D | 42 | 72.667 | 132.352 | 83.615 | 1.00 | 65.74 | D |
| ATOM | 3306 | CB | ALA | D | 42 | 72.924 | 132.857 | 85.007 | 1.00 | 65.68 | D |
| ATOM | 3307 | C | ALA | D | 42 | 71.616 | 131.257 | 83.647 | 1.00 | 66.32 | D |
| ATOM | 3308 | O | ALA | D | 42 | 71.910 | 130.102 | 83.301 | 1.00 | 66.27 | D |
| ATOM | 3309 | N | ARG | D | 43 | 70.406 | 131.639 | 84.082 | 1.00 | 66.54 | D |
| ATOM | 3310 | CA | ARG | D | 43 | 69.259 | 130.751 | 84.177 | 1.00 | 66.79 | D |
| ATOM | 3311 | CB | ARG | D | 43 | 67.976 | 131.580 | 84.320 | 1.00 | 67.47 | D |
| ATOM | 3312 | CG | ARG | D | 43 | 67.849 | 132.416 | 85.593 | 1.00 | 67.13 | D |
| ATOM | 3313 | CD | ARG | D | 43 | 67.654 | 131.528 | 86.768 | 1.00 | 67.30 | D |
| ATOM | 3314 | NE | ARG | D | 43 | 67.301 | 132.304 | 87.937 | 1.00 | 68.47 | D |
| ATOM | 3315 | CZ | ARG | D | 43 | 67.438 | 131.859 | 89.178 | 1.00 | 69.33 | D |
| ATOM | 3316 | NH1 | ARG | D | 43 | 67.924 | 130.635 | 89.388 | 1.00 | 69.94 | D |
| ATOM | 3317 | NH2 | ARG | D | 43 | 67.103 | 132.634 | 90.203 | 1.00 | 69.42 | D |
| ATOM | 3318 | C | ARG | D | 43 | 69.165 | 129.886 | 82.908 | 1.00 | 66.88 | D |
| ATOM | 3319 | O | ARG | D | 43 | 69.104 | 128.652 | 82.973 | 1.00 | 66.89 | D |
| ATOM | 3320 | N | PHE | D | 44 | 69.162 | 130.549 | 81.752 | 1.00 | 66.55 | D |
| ATOM | 3321 | CA | PHE | D | 44 | 69.079 | 129.852 | 80.475 | 1.00 | 66.16 | D |
| ATOM | 3322 | CB | PHE | D | 44 | 69.347 | 130.803 | 79.311 | 1.00 | 63.13 | D |
| ATOM | 3323 | CG | PHE | D | 44 | 69.322 | 130.119 | 77.989 | 1.00 | 61.90 | D |
| ATOM | 3324 | CD1 | PHE | D | 44 | 68.168 | 129.485 | 77.544 | 1.00 | 60.65 | D |
| ATOM | 3325 | CD2 | PHE | D | 44 | 70.461 | 130.020 | 77.223 | 1.00 | 61.36 | D |
| ATOM | 3326 | CE1 | PHE | D | 44 | 68.155 | 128.766 | 76.375 | 1.00 | 58.87 | D |
| ATOM | 3327 | CE2 | PHE | D | 44 | 70.457 | 129.291 | 76.037 | 1.00 | 60.36 | D |
| ATOM | 3328 | CZ | PHE | D | 44 | 69.303 | 128.665 | 75.619 | 1.00 | 60.45 | D |
| ATOM | 3329 | C | PHE | D | 44 | 70.079 | 128.705 | 80.431 | 1.00 | 67.22 | D |
| ATOM | 3330 | O | PHE | D | 44 | 69.693 | 127.532 | 80.378 | 1.00 | 67.33 | D |
| ATOM | 3331 | N | GLN | D | 45 | 71.364 | 129.051 | 80.470 | 1.00 | 68.33 | D |
| ATOM | 3332 | CA | GLN | D | 45 | 72.433 | 128.062 | 80.430 | 1.00 | 69.21 | D |
| ATOM | 3333 | CB | GLN | D | 45 | 73.782 | 128.752 | 80.513 | 1.00 | 70.10 | D |
| ATOM | 3334 | CG | GLN | D | 45 | 74.354 | 129.214 | 79.198 | 1.00 | 70.76 | D |
| ATOM | 3335 | CD | GLN | D | 45 | 75.868 | 129.258 | 79.249 | 1.00 | 71.18 | D |
| ATOM | 3336 | OE1 | GLN | D | 45 | 76.517 | 128.243 | 79.504 | 1.00 | 71.64 | D |
| ATOM | 3337 | NE2 | GLN | D | 45 | 76.434 | 130.432 | 79.014 | 1.00 | 71.03 | D |
| ATOM | 3338 | C | GLN | D | 45 | 72.376 | 126.976 | 81.502 | 1.00 | 68.95 | D |
| ATOM | 3339 | O | GLN | D | 45 | 72.595 | 125.796 | 81.214 | 1.00 | 68.07 | D |
| ATOM | 3340 | N | ASN | D | 46 | 72.118 | 127.366 | 82.738 | 1.00 | 69.16 | D |
| ATOM | 3341 | CA | ASN | D | 46 | 72.025 | 126.371 | 83.798 | 1.00 | 71.01 | D |
| ATOM | 3342 | CB | ASN | D | 46 | 71.520 | 127.027 | 85.077 | 1.00 | 72.95 | D |
| ATOM | 3343 | CG | ASN | D | 46 | 72.312 | 128.313 | 85.429 | 1.00 | 75.88 | D |
| ATOM | 3344 | OD1 | ASN | D | 46 | 73.554 | 128.322 | 85.374 | 1.00 | 76.55 | D |
| ATOM | 3345 | ND2 | ASN | D | 46 | 71.593 | 129.394 | 85.799 | 1.00 | 75.50 | D |
| ATOM | 3346 | C | ASN | D | 46 | 71.064 | 125.259 | 83.370 | 1.00 | 71.10 | D |
| ATOM | 3347 | O | ASN | D | 46 | 71.450 | 124.088 | 83.285 | 1.00 | 70.79 | D |
| ATOM | 3348 | N | ALA | D | 47 | 69.822 | 125.657 | 83.073 | 1.00 | 71.04 | D |
| ATOM | 3349 | CA | ALA | D | 47 | 68.756 | 124.756 | 82.645 | 1.00 | 70.47 | D |
| ATOM | 3350 | CB | ALA | D | 47 | 67.452 | 125.543 | 82.437 | 1.00 | 70.23 | D |
| ATOM | 3351 | C | ALA | D | 47 | 69.122 | 123.990 | 81.373 | 1.00 | 70.45 | D |
| ATOM | 3352 | O | ALA | D | 47 | 68.776 | 122.804 | 81.217 | 1.00 | 69.07 | D |
| ATOM | 3353 | N | CYS | D | 48 | 69.818 | 124.653 | 80.456 | 1.00 | 70.73 | D |
| ATOM | 3354 | CA | CYS | D | 48 | 70.213 | 123.967 | 79.235 | 1.00 | 71.62 | D |
| ATOM | 3355 | CB | CYS | D | 48 | 70.982 | 124.896 | 78.324 | 1.00 | 70.68 | D |
| ATOM | 3356 | SG | CYS | D | 48 | 69.958 | 125.820 | 77.216 | 1.00 | 70.62 | D |
| ATOM | 3357 | C | CYS | D | 48 | 71.079 | 122.775 | 79.589 | 1.00 | 72.73 | D |
| ATOM | 3358 | O | CYS | D | 48 | 70.877 | 121.681 | 79.080 | 1.00 | 73.31 | D |
| ATOM | 3359 | N | ARG | D | 49 | 72.051 | 122.994 | 80.461 | 1.00 | 73.87 | D |
| ATOM | 3360 | CA | ARG | D | 49 | 72.916 | 121.917 | 80.878 | 1.00 | 75.35 | D |
| ATOM | 3361 | CB | ARG | D | 49 | 74.061 | 122.483 | 81.746 | 1.00 | 78.18 | D |
| ATOM | 3362 | CG | ARG | D | 49 | 75.142 | 123.249 | 80.902 | 1.00 | 81.17 | D |
| ATOM | 3363 | CD | ARG | D | 49 | 76.320 | 123.834 | 81.711 | 1.00 | 83.22 | D |
| ATOM | 3364 | NE | ARG | D | 49 | 76.015 | 125.130 | 82.349 | 1.00 | 85.98 | D |
| ATOM | 3365 | CZ | ARG | D | 49 | 75.673 | 125.295 | 83.640 | 1.00 | 86.71 | D |
| ATOM | 3366 | NH1 | ARG | D | 49 | 75.593 | 124.236 | 84.453 | 1.00 | 86.31 | D |
| ATOM | 3367 | NH2 | ARG | D | 49 | 75.400 | 126.522 | 84.123 | 1.00 | 86.39 | D |
| ATOM | 3368 | C | ARG | D | 49 | 72.072 | 120.857 | 81.613 | 1.00 | 75.15 | D |
| ATOM | 3369 | O | ARG | D | 49 | 72.467 | 119.681 | 81.719 | 1.00 | 74.90 | D |
| ATOM | 3370 | N | ASP | D | 50 | 70.900 | 121.271 | 82.096 | 1.00 | 74.47 | D |
| ATOM | 3371 | CA | ASP | D | 50 | 69.968 | 120.353 | 82.763 | 1.00 | 73.42 | D |
| ATOM | 3372 | CB | ASP | D | 50 | 69.015 | 121.123 | 83.696 | 1.00 | 74.98 | D |
| ATOM | 3373 | CG | ASP | D | 50 | 69.540 | 121.251 | 85.115 | 1.00 | 75.45 | D |
| ATOM | 3374 | OD1 | ASP | D | 50 | 69.647 | 120.214 | 85.833 | 1.00 | 75.65 | D |
| ATOM | 3375 | OD2 | ASP | D | 50 | 69.831 | 122.405 | 85.501 | 1.00 | 76.06 | D |
| ATOM | 3376 | C | ASP | D | 50 | 69.126 | 119.615 | 81.690 | 1.00 | 71.52 | D |
| ATOM | 3377 | O | ASP | D | 50 | 68.312 | 118.740 | 82.003 | 1.00 | 71.55 | D |
| ATOM | 3378 | N | GLY | D | 51 | 69.328 | 119.975 | 80.431 | 1.00 | 68.75 | D |
| ATOM | 3379 | CA | GLY | D | 51 | 68.569 | 119.344 | 79.362 | 1.00 | 67.20 | D |
| ATOM | 3380 | C | GLY | D | 51 | 67.147 | 119.871 | 79.164 | 1.00 | 65.47 | D |
| ATOM | 3381 | O | GLY | D | 51 | 66.263 | 119.155 | 78.697 | 1.00 | 64.78 | D |
| ATOM | 3382 | N | ARG | D | 52 | 66.929 | 121.132 | 79.520 | 1.00 | 64.57 | D |
| ATOM | 3383 | CA | ARG | D | 52 | 65.626 | 121.737 | 79.388 | 1.00 | 62.19 | D |
| ATOM | 3384 | CB | ARG | D | 52 | 64.744 | 121.292 | 80.542 | 1.00 | 62.86 | D |
| ATOM | 3385 | CG | ARG | D | 52 | 65.183 | 121.752 | 81.864 | 1.00 | 65.38 | D |
| ATOM | 3386 | CD | ARG | D | 52 | 64.234 | 121.218 | 82.910 | 1.00 | 68.95 | D |
| ATOM | 3387 | NE | ARG | D | 52 | 64.492 | 121.791 | 84.236 | 1.00 | 73.18 | D |
| ATOM | 3388 | CZ | ARG | D | 52 | 64.182 | 123.040 | 84.610 | 1.00 | 75.15 | D |
| ATOM | 3389 | NH1 | ARG | D | 52 | 63.593 | 123.887 | 83.762 | 1.00 | 76.24 | D |
| ATOM | 3390 | NH2 | ARG | D | 52 | 64.447 | 123.450 | 85.848 | 1.00 | 75.97 | D |
| ATOM | 3391 | C | ARG | D | 52 | 65.610 | 123.257 | 79.250 | 1.00 | 60.35 | D |
| ATOM | 3392 | O | ARG | D | 52 | 66.481 | 123.983 | 79.723 | 1.00 | 59.16 | D |
| ATOM | 3393 | N | SER | D | 53 | 64.587 | 123.725 | 78.559 | 1.00 | 58.93 | D |
| ATOM | 3394 | CA | SER | D | 53 | 64.428 | 125.138 | 78.323 | 1.00 | 57.54 | D |
| ATOM | 3395 | CB | SER | D | 53 | 65.509 | 125.637 | 77.400 | 1.00 | 56.75 | D |
| ATOM | 3396 | OG | SER | D | 53 | 65.271 | 126.994 | 77.145 | 1.00 | 56.27 | D |
| ATOM | 3397 | C | SER | D | 53 | 63.098 | 125.452 | 77.698 | 1.00 | 56.81 | D |
| ATOM | 3398 | O | SER | D | 53 | 62.417 | 124.569 | 77.211 | 1.00 | 57.33 | D |
| ATOM | 3399 | N | GLU | D | 54 | 62.743 | 126.728 | 77.698 | 1.00 | 56.26 | D |
| ATOM | 3400 | CA | GLU | D | 54 | 61.492 | 127.195 | 77.122 | 1.00 | 55.56 | D |
| ATOM | 3401 | CB | GLU | D | 54 | 60.659 | 127.911 | 78.166 | 1.00 | 57.06 | D |
| ATOM | 3402 | CG | GLU | D | 54 | 60.315 | 127.104 | 79.367 | 1.00 | 59.88 | D |
| ATOM | 3403 | CD | GLU | D | 54 | 59.824 | 128.000 | 80.481 | 1.00 | 62.26 | D |
| ATOM | 3404 | OE1 | GLU | D | 54 | 59.398 | 129.154 | 80.183 | 1.00 | 63.35 | D |
| ATOM | 3405 | OE2 | GLU | D | 54 | 59.861 | 127.557 | 81.647 | 1.00 | 63.81 | D |
| ATOM | 3406 | C | GLU | D | 54 | 61.764 | 128.191 | 76.022 | 1.00 | 53.73 | D |
| ATOM | 3407 | O | GLU | D | 54 | 62.405 | 129.189 | 76.242 | 1.00 | 52.37 | D |
| ATOM | 3408 | N | ILE | D | 55 | 61.232 | 127.937 | 74.845 | 1.00 | 53.14 | D |
| ATOM | 3409 | CA | ILE | D | 55 | 61.425 | 128.844 | 73.734 | 1.00 | 52.27 | D |
| ATOM | 3410 | CB | ILE | D | 55 | 62.493 | 128.328 | 72.860 | 1.00 | 52.49 | D |
| ATOM | 3411 | CG2 | ILE | D | 55 | 62.651 | 129.207 | 71.675 | 1.00 | 53.54 | D |
| ATOM | 3412 | CG1 | ILE | D | 55 | 63.791 | 128.314 | 73.653 | 1.00 | 54.60 | D |
| ATOM | 3413 | CD1 | ILE | D | 55 | 64.845 | 127.380 | 73.092 | 1.00 | 57.38 | D |
| ATOM | 3414 | C | ILE | D | 55 | 60.158 | 129.061 | 72.920 | 1.00 | 51.96 | D |
| ATOM | 3415 | O | ILE | D | 55 | 59.306 | 128.168 | 72.815 | 1.00 | 52.88 | D |
| ATOM | 3416 | N | ALA | D | 56 | 60.023 | 130.255 | 72.355 | 1.00 | 50.38 | D |
| ATOM | 3417 | CA | ALA | D | 56 | 58.847 | 130.552 | 71.569 | 1.00 | 49.63 | D |
| ATOM | 3418 | CB | ALA | D | 56 | 58.112 | 131.758 | 72.163 | 1.00 | 48.61 | D |
| ATOM | 3419 | C | ALA | D | 56 | 59.192 | 130.831 | 70.115 | 1.00 | 49.54 | D |
| ATOM | 3420 | O | ALA | D | 56 | 60.220 | 131.414 | 69.835 | 1.00 | 50.99 | D |
| ATOM | 3421 | N | PHE | D | 57 | 58.362 | 130.397 | 69.176 | 1.00 | 48.52 | D |
| ATOM | 3422 | CA | PHE | D | 57 | 58.606 | 130.758 | 67.789 | 1.00 | 46.43 | D |
| ATOM | 3423 | CB | PHE | D | 57 | 57.924 | 129.798 | 66.815 | 1.00 | 46.74 | D |
| ATOM | 3424 | CG | PHE | D | 57 | 58.630 | 128.481 | 66.671 | 1.00 | 47.17 | D |
| ATOM | 3425 | CD1 | PHE | D | 57 | 58.231 | 127.378 | 67.385 | 1.00 | 47.26 | D |
| ATOM | 3426 | CD2 | PHE | D | 57 | 59.692 | 128.345 | 65.805 | 1.00 | 47.93 | D |
| ATOM | 3427 | CE1 | PHE | D | 57 | 58.869 | 126.156 | 67.238 | 1.00 | 47.29 | D |
| ATOM | 3428 | CE2 | PHE | D | 57 | 60.336 | 127.120 | 65.654 | 1.00 | 47.62 | D |
| ATOM | 3429 | CZ | PHE | D | 57 | 59.910 | 126.026 | 66.377 | 1.00 | 47.22 | D |
| ATOM | 3430 | C | PHE | D | 57 | 57.928 | 132.132 | 67.756 | 1.00 | 46.16 | D |
| ATOM | 3431 | O | PHE | D | 57 | 56.697 | 132.231 | 67.745 | 1.00 | 45.68 | D |
| ATOM | 3432 | N | VAL | D | 58 | 58.768 | 133.179 | 67.784 | 1.00 | 45.52 | D |
| ATOM | 3433 | CA | VAL | D | 58 | 58.378 | 134.606 | 67.809 | 1.00 | 43.10 | D |
| ATOM | 3434 | CB | VAL | D | 58 | 59.599 | 135.551 | 67.607 | 1.00 | 42.52 | D |
| ATOM | 3435 | CG1 | VAL | D | 58 | 59.151 | 136.956 | 67.618 | 1.00 | 39.32 | D |
| ATOM | 3436 | CG2 | VAL | D | 58 | 60.609 | 135.343 | 68.701 | 1.00 | 42.75 | D |
| ATOM | 3437 | C | VAL | D | 58 | 57.324 | 135.076 | 66.842 | 1.00 | 42.18 | D |
| ATOM | 3438 | O | VAL | D | 58 | 56.386 | 135.736 | 67.244 | 1.00 | 42.83 | D |
| ATOM | 3439 | N | ALA | D | 59 | 57.467 | 134.776 | 65.569 | 1.00 | 40.19 | D |
| ATOM | 3440 | CA | ALA | D | 59 | 56.447 | 135.260 | 64.629 | 1.00 | 41.01 | D |
| ATOM | 3441 | CB | ALA | D | 59 | 56.820 | 134.873 | 63.209 | 1.00 | 38.59 | D |
| ATOM | 3442 | C | ALA | D | 59 | 54.998 | 134.769 | 64.919 | 1.00 | 41.82 | D |
| ATOM | 3443 | O | ALA | D | 59 | 54.030 | 135.536 | 64.810 | 1.00 | 43.03 | D |
| ATOM | 3444 | N | THR | D | 60 | 54.851 | 133.501 | 65.280 | 1.00 | 40.73 | D |
| ATOM | 3445 | CA | THR | D | 60 | 53.532 | 132.973 | 65.538 | 1.00 | 40.43 | D |
| ATOM | 3446 | CB | THR | D | 60 | 53.371 | 131.493 | 65.120 | 1.00 | 39.93 | D |
| ATOM | 3447 | OG1 | THR | D | 60 | 54.224 | 130.698 | 65.938 | 1.00 | 39.88 | D |
| ATOM | 3448 | CG2 | THR | D | 60 | 53.696 | 131.268 | 63.658 | 1.00 | 36.47 | D |
| ATOM | 3449 | C | THR | D | 60 | 53.170 | 132.990 | 66.999 | 1.00 | 40.31 | D |
| ATOM | 3450 | O | THR | D | 60 | 51.982 | 132.867 | 67.355 | 1.00 | 40.70 | D |
| ATOM | 3451 | N | GLY | D | 61 | 54.176 | 133.113 | 67.859 | 1.00 | 39.02 | D |
| ATOM | 3452 | CA | GLY | D | 61 | 53.869 | 133.099 | 69.280 | 1.00 | 36.81 | D |
| ATOM | 3453 | C | GLY | D | 61 | 53.620 | 131.672 | 69.743 | 1.00 | 36.11 | D |
| ATOM | 3454 | O | GLY | D | 61 | 52.974 | 131.456 | 70.741 | 1.00 | 35.32 | D |
| ATOM | 3455 | N | THR | D | 62 | 54.112 | 130.679 | 69.010 | 1.00 | 35.51 | D |
| ATOM | 3456 | CA | THR | D | 62 | 53.965 | 129.314 | 69.475 | 1.00 | 36.22 | D |
| ATOM | 3457 | CB | THR | D | 62 | 54.024 | 128.292 | 68.309 | 1.00 | 36.51 | D |
| ATOM | 3458 | OG1 | THR | D | 62 | 52.933 | 128.546 | 67.421 | 1.00 | 39.34 | D |
| ATOM | 3459 | CG2 | THR | D | 62 | 53.883 | 126.854 | 68.842 | 1.00 | 35.12 | D |
| ATOM | 3460 | C | THR | D | 62 | 55.037 | 128.936 | 70.513 | 1.00 | 36.47 | D |
| ATOM | 3461 | O | THR | D | 62 | 56.199 | 128.780 | 70.173 | 1.00 | 35.40 | D |
| ATOM | 3462 | N | ASN | D | 63 | 54.633 | 128.772 | 71.764 | 1.00 | 35.95 | D |
| ATOM | 3463 | CA | ASN | D | 63 | 55.565 | 128.420 | 72.826 | 1.00 | 37.19 | D |
| ATOM | 3464 | CB | ASN | D | 63 | 55.065 | 128.943 | 74.204 | 1.00 | 37.62 | D |
| ATOM | 3465 | CG | ASN | D | 63 | 55.111 | 130.483 | 74.320 | 1.00 | 38.81 | D |
| ATOM | 3466 | OD1 | ASN | D | 63 | 55.777 | 131.033 | 75.204 | 1.00 | 40.38 | D |
| ATOM | 3467 | ND2 | ASN | D | 63 | 54.411 | 131.169 | 73.431 | 1.00 | 38.85 | D |
| ATOM | 3468 | C | ASN | D | 63 | 55.746 | 126.906 | 72.919 | 1.00 | 38.57 | D |
| ATOM | 3469 | O | ASN | D | 63 | 54.855 | 126.139 | 72.507 | 1.00 | 37.97 | D |
| ATOM | 3470 | N | LEU | D | 64 | 56.898 | 126.493 | 73.468 | 1.00 | 38.79 | D |
| ATOM | 3471 | CA | LEU | D | 64 | 57.246 | 125.079 | 73.689 | 1.00 | 38.20 | D |
| ATOM | 3472 | CB | LEU | D | 64 | 57.931 | 124.480 | 72.449 | 1.00 | 36.63 | D |
| ATOM | 3473 | CG | LEU | D | 64 | 57.137 | 124.259 | 71.128 | 1.00 | 37.55 | D |
| ATOM | 3474 | CD1 | LEU | D | 64 | 58.074 | 123.741 | 70.043 | 1.00 | 34.33 | D |
| ATOM | 3475 | CD2 | LEU | D | 64 | 55.928 | 123.269 | 71.347 | 1.00 | 35.66 | D |
| ATOM | 3476 | C | LEU | D | 64 | 58.218 | 124.942 | 74.861 | 1.00 | 39.26 | D |
| ATOM | 3477 | O | LEU | D | 64 | 59.076 | 125.807 | 75.061 | 1.00 | 38.92 | D |
| ATOM | 3478 | N | SER | D | 65 | 58.048 | 123.910 | 75.684 | 1.00 | 39.66 | D |
| ATOM | 3479 | CA | SER | D | 65 | 59.007 | 123.641 | 76.738 | 1.00 | 40.30 | D |
| ATOM | 3480 | CB | SER | D | 65 | 58.381 | 123.097 | 78.009 | 1.00 | 42.49 | D |
| ATOM | 3481 | OG | SER | D | 65 | 57.697 | 124.069 | 78.760 | 1.00 | 47.61 | D |
| ATOM | 3482 | C | SER | D | 65 | 59.709 | 122.478 | 76.042 | 1.00 | 41.65 | D |
| ATOM | 3483 | O | SER | D | 65 | 59.022 | 121.614 | 75.451 | 1.00 | 40.41 | D |
| ATOM | 3484 | N | LEU | D | 66 | 61.048 | 122.443 | 76.134 | 1.00 | 42.73 | D |
| ATOM | 3485 | CA | LEU | D | 66 | 61.852 | 121.425 | 75.487 | 1.00 | 44.33 | D |
| ATOM | 3486 | CB | LEU | D | 66 | 62.814 | 122.095 | 74.534 | 1.00 | 43.28 | D |
| ATOM | 3487 | CG | LEU | D | 66 | 62.092 | 122.993 | 73.539 | 1.00 | 43.51 | D |
| ATOM | 3488 | CD1 | LEU | D | 66 | 63.082 | 123.861 | 72.733 | 1.00 | 41.22 | D |
| ATOM | 3489 | CD2 | LEU | D | 66 | 61.222 | 122.086 | 72.645 | 1.00 | 42.23 | D |
| ATOM | 3490 | C | LEU | D | 66 | 62.634 | 120.561 | 76.435 | 1.00 | 46.85 | D |
| ATOM | 3491 | O | LEU | D | 66 | 63.049 | 121.011 | 77.482 | 1.00 | 48.18 | D |
| ATOM | 3492 | N | GLN | D | 67 | 62.815 | 119.302 | 76.061 | 1.00 | 49.89 | D |
| ATOM | 3493 | CA | GLN | D | 67 | 63.593 | 118.334 | 76.833 | 1.00 | 52.13 | D |
| ATOM | 3494 | CB | GLN | D | 67 | 62.753 | 117.153 | 77.197 | 1.00 | 53.69 | D |
| ATOM | 3495 | CG | GLN | D | 67 | 61.589 | 117.497 | 78.001 | 1.00 | 56.00 | D |
| ATOM | 3496 | CD | GLN | D | 67 | 61.708 | 116.920 | 79.377 | 1.00 | 58.21 | D |
| ATOM | 3497 | OE1 | GLN | D | 67 | 61.946 | 117.662 | 80.352 | 1.00 | 58.68 | D |
| ATOM | 3498 | NE2 | GLN | D | 67 | 61.554 | 115.576 | 79.480 | 1.00 | 57.34 | D |
| ATOM | 3499 | C | GLN | D | 67 | 64.602 | 117.842 | 75.823 | 1.00 | 53.74 | D |
| ATOM | 3500 | O | GLN | D | 67 | 64.220 | 117.270 | 74.805 | 1.00 | 53.80 | D |
| ATOM | 3501 | N | PHE | D | 68 | 65.882 | 118.050 | 76.093 | 1.00 | 55.63 | D |
| ATOM | 3502 | CA | PHE | D | 68 | 66.916 | 117.631 | 75.157 | 1.00 | 56.56 | D |
| ATOM | 3503 | CB | PHE | D | 68 | 68.008 | 118.681 | 75.108 | 1.00 | 54.80 | D |
| ATOM | 3504 | CG | PHE | D | 68 | 67.502 | 120.083 | 75.151 | 1.00 | 53.24 | D |
| ATOM | 3505 | CD1 | PHE | D | 68 | 67.747 | 120.899 | 76.279 | 1.00 | 53.65 | D |
| ATOM | 3506 | CD2 | PHE | D | 68 | 66.837 | 120.618 | 74.062 | 1.00 | 52.08 | D |
| ATOM | 3507 | CE1 | PHE | D | 68 | 67.337 | 122.236 | 76.295 | 1.00 | 52.73 | D |
| ATOM | 3508 | CE2 | PHE | D | 68 | 66.421 | 121.940 | 74.065 | 1.00 | 51.75 | D |
| ATOM | 3509 | CZ | PHE | D | 68 | 66.672 | 122.755 | 75.177 | 1.00 | 52.13 | D |
| ATOM | 3510 | C | PHE | D | 68 | 67.531 | 116.283 | 75.523 | 1.00 | 58.25 | D |
| ATOM | 3511 | O | PHE | D | 68 | 68.646 | 116.224 | 76.024 | 1.00 | 59.67 | D |
| ATOM | 3512 | N | PHE | D | 69 | 66.788 | 115.213 | 75.298 | 1.00 | 59.90 | D |
| ATOM | 3513 | CA | PHE | D | 69 | 67.263 | 113.863 | 75.549 | 1.00 | 62.39 | D |
| ATOM | 3514 | CB | PHE | D | 69 | 66.796 | 113.317 | 76.884 | 1.00 | 63.98 | D |
| ATOM | 3515 | CG | PHE | D | 69 | 67.028 | 114.235 | 78.027 | 1.00 | 66.81 | D |
| ATOM | 3516 | CD1 | PHE | D | 69 | 66.068 | 115.168 | 78.379 | 1.00 | 68.12 | D |
| ATOM | 3517 | CD2 | PHE | D | 69 | 68.214 | 114.163 | 78.776 | 1.00 | 68.82 | D |
| ATOM | 3518 | CE1 | PHE | D | 69 | 66.268 | 116.027 | 79.470 | 1.00 | 70.30 | D |
| ATOM | 3519 | CE2 | PHE | D | 69 | 68.439 | 115.019 | 79.877 | 1.00 | 70.28 | D |
| ATOM | 3520 | CZ | PHE | D | 69 | 67.465 | 115.953 | 80.228 | 1.00 | 70.63 | D |
| ATOM | 3521 | C | PHE | D | 69 | 66.567 | 113.084 | 74.471 | 1.00 | 63.60 | D |
| ATOM | 3522 | O | PHE | P | 69 | 65.616 | 113.581 | 73.861 | 1.00 | 63.87 | D |
| ATOM | 3523 | N | PRO | D | 70 | 67.019 | 111.859 | 74.207 | 1.00 | 64.24 | D |
| ATOM | 3524 | CD | PRO | D | 70 | 68.296 | 111.261 | 74.626 | 1.00 | 64.07 | D |
| ATOM | 3525 | CA | PRO | D | 70 | 66.361 | 111.065 | 73.162 | 1.00 | 65.05 | D |
| ATOM | 3526 | CB | PRO | D | 70 | 67.426 | 110.036 | 72.796 | 1.00 | 64.31 | D |
| ATOM | 3527 | CG | PRO | D | 70 | 68.163 | 109.861 | 74.101 | 1.00 | 64.14 | D |
| ATOM | 3528 | C | PRO | D | 70 | 65.069 | 110.461 | 73.713 | 1.00 | 66.02 | D |
| ATOM | 3529 | O | PRO | D | 70 | 64.969 | 110.222 | 74.908 | 1.00 | 65.84 | D |
| ATOM | 3530 | N | ALA | D | 71 | 64.059 | 110.248 | 72.881 | 1.00 | 67.68 | D |
| ATOM | 3531 | CA | ALA | D | 71 | 62.845 | 109.666 | 73.428 | 1.00 | 69.88 | D |
| ATOM | 3532 | CB | ALA | D | 71 | 61.924 | 109.243 | 72.305 | 1.00 | 70.30 | D |
| ATOM | 3533 | C | ALA | D | 71 | 63.231 | 108.452 | 74.314 | 1.00 | 71.42 | D |
| ATOM | 3534 | O | ALA | D | 71 | 64.111 | 107.642 | 73.930 | 1.00 | 70.45 | D |
| ATOM | 3535 | N | SER | D | 72 | 62.596 | 108.369 | 75.498 | 1.00 | 73.17 | D |
| ATOM | 3536 | CA | SER | D | 72 | 62.803 | 107.298 | 76.518 | 1.00 | 74.70 | D |
| ATOM | 3537 | CB | SER | D | 72 | 63.455 | 106.040 | 75.905 | 1.00 | 75.10 | D |
| ATOM | 3538 | OG | SER | D | 72 | 63.668 | 105.027 | 76.878 | 1.00 | 74.67 | D |
| ATOM | 3539 | C | SER | D | 72 | 63.647 | 107.726 | 77.730 | 1.00 | 75.71 | D |
| ATOM | 3540 | O | SER | D | 72 | 64.874 | 107.880 | 77.628 | 1.00 | 75.99 | D |
| ATOM | 3541 | N | ALA | D | 73 | 62.991 | 107.902 | 78.873 | 0.00 | 75.89 | D |
| ATOM | 3542 | CA | ALA | D | 73 | 63.684 | 108.302 | 80.092 | 0.00 | 76.20 | D |
| ATOM | 3543 | CB | ALA | D | 73 | 63.808 | 109.819 | 80.147 | 0.00 | 76.22 | D |
| ATOM | 3544 | C | ALA | D | 73 | 62.958 | 107.791 | 81.332 | 0.00 | 76.37 | D |
| ATOM | 3545 | O | ALA | D | 73 | 61.737 | 107.637 | 81.328 | 0.00 | 76.42 | D |
| ATOM | 3546 | N | ALA | D | 74 | 63.719 | 107.528 | 82.390 | 0.00 | 76.55 | D |
| ATOM | 3547 | CA | ALA | D | 74 | 63.159 | 107.035 | 83.642 | 0.00 | 76.67 | D |
| ATOM | 3548 | CB | ALA | D | 74 | 62.384 | 108.149 | 84.339 | 0.00 | 76.73 | D |
| ATOM | 3549 | C | ALA | D | 74 | 62.251 | 105.832 | 83.408 | 0.00 | 76.74 | D |
| ATOM | 3550 | O | ALA | D | 74 | 61.080 | 105.840 | 83.792 | 0.00 | 76.80 | D |
| ATOM | 3551 | N | THR | D | 80 | 71.743 | 113.881 | 82.679 | 1.00 | 85.10 | D |
| ATOM | 3552 | CA | THR | D | 80 | 72.533 | 114.923 | 82.007 | 1.00 | 85.01 | D |
| ATOM | 3553 | CB | THR | D | 80 | 74.036 | 114.909 | 82.451 | 1.00 | 84.53 | D |
| ATOM | 3554 | OG1 | THR | D | 80 | 74.139 | 115.347 | 83.808 | 1.00 | 82.90 | D |
| ATOM | 3555 | CG2 | THR | D | 80 | 74.875 | 115.841 | 81.574 | 1.00 | 84.01 | D |
| ATOM | 3556 | C | THR | D | 80 | 72.472 | 114.788 | 80.480 | 1.00 | 85.21 | D |
| ATOM | 3557 | O | THR | D | 80 | 72.542 | 113.691 | 79.915 | 1.00 | 84.93 | D |
| ATOM | 3558 | N | PRO | D | 81 | 72.334 | 115.919 | 79.792 | 1.00 | 85.20 | D |
| ATOM | 3559 | CD | PRO | D | 81 | 72.143 | 117.279 | 80.338 | 1.00 | 85.40 | D |
| ATOM | 3560 | CA | PRO | D | 81 | 72.262 | 115.903 | 78.330 | 1.00 | 85.23 | D |
| ATOM | 3561 | CB | PRO | D | 81 | 71.829 | 117.337 | 77.992 | 1.00 | 85.34 | D |
| ATOM | 3562 | CG | PRO | D | 81 | 72.417 | 118.168 | 79.129 | 1.00 | 85.50 | D |
| ATOM | 3563 | C | PRO | D | 81 | 73.572 | 115.506 | 77.645 | 1.00 | 85.03 | D |
| ATOM | 3564 | O | PRO | D | 81 | 74.627 | 115.494 | 78.275 | 1.00 | 84.92 | D |
| ATOM | 3565 | N | SER | D | 82 | 73.479 | 115.195 | 76.353 | 1.00 | 84.87 | D |
| ATOM | 3566 | CA | SER | D | 82 | 74.619 | 114.810 | 75.528 | 1.00 | 84.86 | D |
| ATOM | 3567 | CB | SER | D | 82 | 74.267 | 113.610 | 74.652 | 1.00 | 84.82 | D |
| ATOM | 3568 | OG | SER | D | 82 | 74.807 | 113.781 | 73.350 | 1.00 | 84.22 | D |
| ATOM | 3569 | C | SER | D | 82 | 74.968 | 115.964 | 74.617 | 1.00 | 85.17 | D |
| ATOM | 3570 | O | SER | D | 82 | 74.127 | 116.813 | 74.361 | 1.00 | 85.56 | D |
| ATOM | 3571 | N | ARG | D | 83 | 76.192 | 115.981 | 74.100 | 1.00 | 85.39 | D |
| ATOM | 3572 | CA | ARG | D | 83 | 76.599 | 117.064 | 73.209 | 1.00 | 85.89 | D |
| ATOM | 3573 | CB | ARG | D | 83 | 78.089 | 116.922 | 72.811 | 1.00 | 88.09 | D |
| ATOM | 3574 | CG | ARG | D | 83 | 78.731 | 118.198 | 72.225 | 1.00 | 90.48 | D |
| ATOM | 3575 | CD | ARG | D | 83 | 78.695 | 119.376 | 73.247 | 1.00 | 93.40 | D |
| ATOM | 3576 | NE | ARG | D | 83 | 79.855 | 119.434 | 74.158 | 1.00 | 96.34 | D |
| ATOM | 3577 | CZ | ARG | D | 83 | 80.114 | 118.571 | 75.146 | 1.00 | 97.40 | D |
| ATOM | 3578 | NH1 | ARG | D | 83 | 79.289 | 117.545 | 75.397 | 1.00 | 97.52 | D |
| ATOM | 3579 | NH2 | ARG | D | 83 | 81.225 | 118.725 | 75.869 | 1.00 | 97.41 | D |
| ATOM | 3580 | C | ARG | D | 83 | 75.704 | 117.013 | 71.968 | 1.00 | 84.73 | D |
| ATOM | 3581 | O | ARG | D | 83 | 75.532 | 118.018 | 71.278 | 1.00 | 84.59 | D |
| ATOM | 3582 | N | GLU | D | 84 | 75.127 | 115.839 | 71.705 | 1.00 | 83.13 | D |
| ATOM | 3583 | CA | GLU | D | 84 | 74.253 | 115.647 | 70.544 | 1.00 | 81.45 | D |
| ATOM | 3584 | CB | GLU | D | 84 | 73.858 | 114.176 | 70.392 | 1.00 | 80.74 | D |
| ATOM | 3585 | CG | GLU | D | 84 | 73.001 | 113.898 | 69.160 | 1.00 | 79.13 | D |
| ATOM | 3586 | CD | GLU | D | 84 | 72.538 | 112.443 | 69.065 | 1.00 | 77.99 | D |
| ATOM | 3587 | OE1 | GLU | D | 84 | 73.182 | 111.580 | 69.692 | 1.00 | 77.10 | D |
| ATOM | 3588 | OE2 | GLU | D | 84 | 71.546 | 112.163 | 68.348 | 1.00 | 76.34 | D |
| ATOM | 3589 | C | GLU | D | 84 | 72.985 | 116.455 | 70.676 | 1.00 | 80.87 | D |
| ATOM | 3590 | O | GLU | D | 84 | 72.454 | 116.935 | 69.668 | 1.00 | 80.47 | D |
| ATOM | 3591 | N | TYR | D | 85 | 72.504 | 116.572 | 71.922 | 1.00 | 80.17 | D |
| ATOM | 3592 | CA | TYR | D | 85 | 71.273 | 117.308 | 72.245 | 1.00 | 79.72 | D |
| ATOM | 3593 | CB | TYR | D | 85 | 70.426 | 116.490 | 73.226 | 1.00 | 78.60 | D |
| ATOM | 3594 | CG | TYR | D | 85 | 69.792 | 115.308 | 72.515 | 1.00 | 78.24 | D |
| ATOM | 3595 | CD1 | TYR | D | 85 | 70.483 | 114.104 | 72.347 | 1.00 | 78.22 | D |
| ATOM | 3596 | CE1 | TYR | D | 85 | 69.942 | 113.063 | 71.595 | 1.00 | 77.54 | D |
| ATOM | 3597 | CD2 | TYR | D | 85 | 68.541 | 115.432 | 71.917 | 1.00 | 77.93 | D |
| ATOM | 3598 | CE2 | TYR | D | 85 | 67.994 | 114.406 | 71.166 | 1.00 | 77.46 | D |
| ATOM | 3599 | CZ | TYR | D | 85 | 68.696 | 113.230 | 71.007 | 1.00 | 77.62 | D |
| ATOM | 3600 | OH | TYR | D | 85 | 68.139 | 112.248 | 70.229 | 1.00 | 77.21 | D |
| ATOM | 3601 | C | TYR | D | 85 | 71.500 | 118.733 | 72.752 | 1.00 | 79.44 | D |
| ATOM | 3602 | O | TYR | D | 85 | 70.739 | 119.650 | 72.412 | 1.00 | 79.51 | D |
| ATOM | 3603 | N | VAL | D | 86 | 72.555 | 118.906 | 73.545 | 1.00 | 79.06 | D |
| ATOM | 3604 | CA | VAL | D | 86 | 72.966 | 120.214 | 74.070 | 1.00 | 78.82 | D |
| ATOM | 3605 | CB | VAL | D | 86 | 72.922 | 120.267 | 75.627 | 1.00 | 77.98 | D |
| ATOM | 3606 | CG1 | VAL | D | 86 | 73.418 | 121.611 | 76.123 | 1.00 | 76.13 | D |
| ATOM | 3607 | CG2 | VAL | D | 86 | 71.506 | 120.001 | 76.118 | 1.00 | 77.42 | D |
| ATOM | 3608 | C | VAL | D | 86 | 74.421 | 120.380 | 73.600 | 1.00 | 79.25 | D |
| ATOM | 3609 | O | VAL | D | 86 | 75.358 | 119.942 | 74.278 | 1.00 | 79.57 | D |
| ATOM | 3610 | N | ASP | D | 87 | 74.600 | 121.000 | 72.437 | 1.00 | 78.86 | D |
| ATOM | 3611 | CA | ASP | D | 87 | 75.927 | 121.185 | 71.873 | 1.00 | 78.79 | D |
| ATOM | 3612 | CB | ASP | D | 87 | 75.917 | 120.955 | 70.363 | 1.00 | 79.00 | D |
| ATOM | 3613 | CG | ASP | D | 87 | 77.305 | 120.821 | 69.793 | 1.00 | 78.80 | D |
| ATOM | 3614 | OD1 | ASP | D | 87 | 78.237 | 121.348 | 70.413 | 1.00 | 78.79 | D |
| ATOM | 3615 | OD2 | ASP | D | 87 | 77.469 | 120.197 | 68.725 | 1.00 | 79.28 | D |
| ATOM | 3616 | C | ASP | D | 87 | 76.483 | 122.556 | 72.122 | 1.00 | 78.99 | D |
| ATOM | 3617 | O | ASP | D | 87 | 76.040 | 123.537 | 71.520 | 1.00 | 78.60 | D |
| ATOM | 3618 | N | LEU | D | 88 | 77.490 | 122.613 | 72.983 | 1.00 | 79.67 | D |
| ATOM | 3619 | CA | LEU | D | 88 | 78.142 | 123.875 | 73.300 | 1.00 | 80.27 | D |
| ATOM | 3620 | CB | LEU | D | 88 | 78.428 | 123.936 | 74.794 | 1.00 | 79.99 | D |
| ATOM | 3621 | CG | LEU | D | 88 | 77.218 | 124.536 | 75.513 | 1.00 | 80.38 | D |
| ATOM | 3622 | CD1 | LEU | D | 88 | 77.414 | 124.542 | 77.017 | 1.00 | 80.83 | D |
| ATOM | 3623 | CD2 | LEU | D | 88 | 77.011 | 125.965 | 74.994 | 1.00 | 80.71 | D |
| ATOM | 3624 | C | LEU | D | 88 | 79.416 | 124.209 | 72.503 | 1.00 | 80.19 | D |
| ATOM | 3625 | O | LEU | D | 88 | 79.774 | 125.375 | 72.381 | 1.00 | 80.33 | D |
| ATOM | 3626 | N | GLU | D | 89 | 80.079 | 123.196 | 71.952 | 1.00 | 80.52 | D |
| ATOM | 3627 | CA | GLU | D | 89 | 81.311 | 123.383 | 71.178 | 1.00 | 80.73 | D |
| ATOM | 3628 | CB | GLU | D | 89 | 82.034 | 122.036 | 71.047 | 1.00 | 82.50 | D |
| ATOM | 3629 | CG | GLU | D | 89 | 82.247 | 121.327 | 72.412 | 1.00 | 86.00 | D |
| ATOM | 3630 | CD | GLU | D | 89 | 82.618 | 119.838 | 72.292 | 1.00 | 88.55 | D |
| ATOM | 3631 | OE1 | GLU | D | 89 | 81.783 | 119.022 | 71.796 | 1.00 | 89.52 | D |
| ATOM | 3632 | OE2 | GLU | D | 89 | 83.755 | 119.488 | 72.698 | 1.00 | 89.79 | D |
| ATOM | 3633 | C | GLU | D | 89 | 81.008 | 123.968 | 69.800 | 1.00 | 79.69 | D |
| ATOM | 3634 | O | GLU | D | 89 | 81.196 | 125.150 | 69.569 | 1.00 | 79.11 | D |
| ATOM | 3635 | N | ARG | D | 90 | 80.545 | 123.121 | 68.894 | 1.00 | 78.91 | D |
| ATOM | 3636 | CA | ARG | D | 90 | 80.169 | 123.505 | 67.538 | 1.00 | 78.33 | D |
| ATOM | 3637 | CB | ARG | D | 90 | 78.707 | 123.133 | 67.321 | 1.00 | 77.44 | D |
| ATOM | 3638 | CG | ARG | D | 90 | 78.128 | 123.714 | 66.082 | 1.00 | 76.98 | D |
| ATOM | 3639 | CD | ARG | D | 90 | 76.939 | 122.937 | 65.678 | 1.00 | 77.57 | D |
| ATOM | 3640 | NE | ARG | D | 90 | 76.359 | 123.489 | 64.466 | 1.00 | 78.58 | D |
| ATOM | 3641 | CZ | ARG | D | 90 | 75.488 | 122.842 | 63.706 | 1.00 | 79.19 | D |
| ATOM | 3642 | NH1 | ARG | D | 90 | 75.105 | 121.609 | 64.041 | 1.00 | 79.71 | D |
| ATOM | 3643 | NH2 | ARG | D | 90 | 74.995 | 123.434 | 62.625 | 1.00 | 79.28 | D |
| ATOM | 3644 | C | ARG | D | 90 | 80.395 | 124.958 | 67.083 | 1.00 | 78.13 | D |
| ATOM | 3645 | O | ARG | D | 90 | 80.971 | 125.202 | 66.020 | 1.00 | 78.14 | D |
| ATOM | 3646 | N | GLU | D | 91 | 79.892 | 125.909 | 67.860 | 1.00 | 77.91 | D |
| ATOM | 3647 | CA | GLU | D | 91 | 80.035 | 127.332 | 67.565 | 1.00 | 77.60 | D |
| ATOM | 3648 | CB | GLU | D | 91 | 78.921 | 127.805 | 66.631 | 1.00 | 76.44 | D |
| ATOM | 3649 | CG | GLU | D | 91 | 78.967 | 129.284 | 66.309 | 1.00 | 76.02 | D |
| ATOM | 3650 | CD | GLU | D | 91 | 78.237 | 129.586 | 65.026 | 1.00 | 76.63 | D |
| ATOM | 3651 | OE1 | GLU | D | 91 | 78.004 | 130.781 | 64.721 | 1.00 | 76.32 | D |
| ATOM | 3652 | OE2 | GLU | D | 91 | 77.903 | 128.609 | 64.315 | 1.00 | 76.76 | D |
| ATOM | 3653 | C | GLU | D | 91 | 79.978 | 128.062 | 68.917 | 1.00 | 78.28 | D |
| ATOM | 3654 | O | GLU | D | 91 | 79.152 | 127.745 | 69.778 | 1.00 | 78.42 | D |
| ATOM | 3655 | N | ALA | D | 92 | 80.874 | 129.022 | 69.128 | 1.00 | 78.46 | D |
| ATOM | 3656 | CA | ALA | D | 92 | 80.898 | 129.723 | 70.403 | 1.00 | 77.60 | D |
| ATOM | 3657 | CB | ALA | D | 92 | 82.302 | 130.199 | 70.726 | 1.00 | 77.77 | D |
| ATOM | 3658 | C | ALA | D | 92 | 79.952 | 130.899 | 70.364 | 1.00 | 77.52 | D |
| ATOM | 3659 | O | ALA | D | 92 | 79.650 | 131.448 | 69.283 | 1.00 | 76.69 | D |
| ATOM | 3660 | N | GLY | D | 93 | 79.498 | 131.283 | 71.561 | 1.00 | 76.87 | D |
| ATOM | 3661 | CA | GLY | D | 93 | 78.556 | 132.390 | 71.686 | 1.00 | 76.45 | D |
| ATOM | 3662 | C | GLY | D | 93 | 77.086 | 131.956 | 71.562 | 1.00 | 76.07 | D |
| ATOM | 3663 | O | GLY | D | 93 | 76.151 | 132.733 | 71.887 | 1.00 | 76.41 | D |
| ATOM | 3664 | N | LYS | D | 94 | 76.889 | 130.719 | 71.084 | 1.00 | 73.97 | D |
| ATOM | 3665 | CA | LYS | D | 94 | 75.567 | 130.160 | 70.904 | 1.00 | 71.80 | D |
| ATOM | 3666 | CB | LYS | D | 94 | 75.041 | 130.541 | 69.519 | 1.00 | 71.38 | D |
| ATOM | 3667 | CG | LYS | D | 94 | 76.001 | 130.294 | 68.408 | 1.00 | 70.71 | D |
| ATOM | 3668 | CD | LYS | D | 94 | 75.567 | 131.056 | 67.176 | 1.00 | 70.61 | D |
| ATOM | 3669 | CE | LYS | D | 94 | 74.795 | 130.164 | 66.219 | 1.00 | 71.83 | D |
| ATOM | 3670 | NZ | LYS | D | 94 | 74.620 | 130.771 | 64.820 | 1.00 | 72.10 | D |
| ATOM | 3671 | C | LYS | D | 94 | 75.506 | 128.641 | 71.100 | 1.00 | 70.46 | D |
| ATOM | 3672 | O | LYS | D | 94 | 76.472 | 127.922 | 70.815 | 1.00 | 70.79 | D |
| ATOM | 3673 | N | VAL | D | 95 | 74.366 | 128.166 | 71.605 | 1.00 | 67.61 | D |
| ATOM | 3674 | CA | VAL | D | 95 | 74.164 | 126.745 | 71.820 | 1.00 | 65.02 | D |
| ATOM | 3675 | CB | VAL | D | 95 | 73.567 | 126.469 | 73.200 | 1.00 | 64.30 | D |
| ATOM | 3676 | CG1 | VAL | D | 95 | 72.159 | 127.010 | 73.265 | 1.00 | 64.40 | D |
| ATOM | 3677 | CG2 | VAL | D | 95 | 73.582 | 124.989 | 73.493 | 1.00 | 63.25 | D |
| ATOM | 3678 | C | VAL | D | 95 | 73.239 | 126.144 | 70.749 | 1.00 | 63.66 | D |
| ATOM | 3679 | O | VAL | D | 95 | 72.332 | 126.794 | 70.242 | 1.00 | 64.34 | D |
| ATOM | 3680 | N | TYR | D | 96 | 73.506 | 124.902 | 70.378 | 1.00 | 61.32 | D |
| ATOM | 3681 | CA | TYR | D | 96 | 72.695 | 124.227 | 69.402 | 1.00 | 59.10 | D |
| ATOM | 3682 | CB | TYR | D | 96 | 73.571 | 123.596 | 68.356 | 1.00 | 58.32 | D |
| ATOM | 3683 | CG | TYR | D | 96 | 73.911 | 124.577 | 67.284 | 1.00 | 57.24 | D |
| ATOM | 3684 | CD1 | TYR | D | 96 | 74.660 | 125.724 | 67.571 | 1.00 | 57.36 | D |
| ATOM | 3685 | CE1 | TYR | D | 96 | 74.965 | 126.670 | 66.564 | 1.00 | 57.26 | D |
| ATOM | 3686 | CD2 | TYR | D | 96 | 73.471 | 124.377 | 65.978 | 1.00 | 56.60 | D |
| ATOM | 3687 | CE2 | TYR | D | 96 | 73.763 | 125.300 | 64.973 | 1.00 | 56.83 | D |
| ATOM | 3688 | CZ | TYR | D | 96 | 74.500 | 126.451 | 65.270 | 1.00 | 56.39 | D |
| ATOM | 3689 | OH | TYR | D | 96 | 74.666 | 127.406 | 64.286 | 1.00 | 56.32 | D |
| ATOM | 3690 | C | TYR | D | 96 | 71.917 | 123.184 | 70.150 | 1.00 | 57.88 | D |
| ATOM | 3691 | O | TYR | D | 96 | 72.486 | 122.394 | 70.908 | 1.00 | 57.88 | P |
| ATOM | 3692 | N | LEU | D | 97 | 70.608 | 123.175 | 69.950 | 1.00 | 56.44 | D |
| ATOM | 3693 | CA | LEU | D | 97 | 69.788 | 122.220 | 70.683 | 1.00 | 54.85 | D |
| ATOM | 3694 | CB | LEU | D | 97 | 68.862 | 122.982 | 71.602 | 1.00 | 54.20 | D |
| ATOM | 3695 | CG | LEU | D | 97 | 69.621 | 123.938 | 72.487 | 1.00 | 53.28 | D |
| ATOM | 3696 | CD1 | LEU | D | 97 | 68.759 | 125.128 | 72.744 | 1.00 | 53.40 | D |
| ATOM | 3697 | CD2 | LEU | D | 97 | 70.016 | 123.241 | 73.765 | 1.00 | 52.86 | D |
| ATOM | 3698 | C | LEU | D | 97 | 68.975 | 121.284 | 69.815 | 1.00 | 53.32 | D |
| ATOM | 3699 | O | LEU | D | 97 | 68.716 | 121.559 | 68.647 | 1.00 | 52.62 | D |
| ATOM | 3700 | N | LYS | D | 98 | 68.572 | 120.177 | 70.415 | 1.00 | 51.98 | D |
| ATOM | 3701 | CA | LYS | D | 98 | 67.764 | 119.176 | 69.732 | 1.00 | 51.09 | D |
| ATOM | 3702 | CB | LYS | D | 98 | 68.679 | 118.069 | 69.197 | 1.00 | 53.07 | D |
| ATOM | 3703 | CG | LYS | D | 98 | 68.051 | 117.068 | 68.213 | 1.00 | 55.64 | D |
| ATOM | 3704 | CD | LYS | D | 98 | 69.002 | 115.851 | 67.995 | 1.00 | 57.58 | D |
| ATOM | 3705 | CE | LYS | D | 98 | 68.306 | 114.789 | 67.147 | 1.00 | 59.64 | D |
| ATOM | 3706 | NZ | LYS | D | 98 | 68.923 | 113.436 | 67.276 | 1.00 | 60.87 | D |
| ATOM | 3707 | C | LYS | D | 98 | 66.765 | 118.619 | 70.778 | 1.00 | 48.82 | D |
| ATOM | 3708 | O | LYS | D | 98 | 67.154 | 118.216 | 71.888 | 1.00 | 46.45 | D |
| ATOM | 3709 | N | ALA | D | 99 | 65.471 | 118.626 | 70.441 | 1.00 | 47.03 | D |
| ATOM | 3710 | CA | ALA | D | 99 | 64.468 | 118.113 | 71.375 | 1.00 | 43.93 | D |
| ATOM | 3711 | CB | ALA | D | 99 | 63.922 | 119.229 | 72.192 | 1.00 | 44.05 | D |
| ATOM | 3712 | C | ALA | D | 99 | 63.344 | 117.389 | 70.692 | 1.00 | 42.49 | D |
| ATOM | 3713 | O | ALA | D | 99 | 62.574 | 117.969 | 69.919 | 1.00 | 43.23 | D |
| ATOM | 3714 | N | PRO | D | 100 | 63.253 | 116.084 | 70.927 | 1.00 | 41.88 | D |
| ATOM | 3715 | CD | PRO | D | 100 | 64.191 | 115.230 | 71.678 | 1.00 | 41.19 | D |
| ATOM | 3716 | CA | PRO | D | 100 | 62.179 | 115.288 | 70.313 | 1.00 | 41.25 | D |
| ATOM | 3717 | CB | PRO | D | 100 | 62.761 | 113.889 | 70.315 | 1.00 | 39.88 | D |
| ATOM | 3718 | CG | PRO | D | 100 | 63.515 | 113.880 | 71.621 | 1.00 | 40.93 | D |
| ATOM | 3719 | C | PRO | D | 100 | 60.929 | 115.398 | 71.210 | 1.00 | 40.41 | D |
| ATOM | 3720 | O | PRO | D | 100 | 61.083 | 115.567 | 72.433 | 1.00 | 40.22 | D |
| ATOM | 3721 | N | MET | D | 101 | 59.731 | 115.362 | 70.603 | 1.00 | 39.05 | D |
| ATOM | 3722 | CA | MET | D | 101 | 58.444 | 115.400 | 71.329 | 1.00 | 38.66 | D |
| ATOM | 3723 | CB | MET | D | 101 | 58.108 | 116.814 | 71.796 | 1.00 | 38.99 | D |
| ATOM | 3724 | CG | MET | D | 101 | 57.482 | 117.688 | 70.709 | 1.00 | 39.81 | D |
| ATOM | 3725 | SD | MET | D | 101 | 58.165 | 119.389 | 70.749 | 1.00 | 46.04 | D |
| ATOM | 3726 | CE | MET | D | 101 | 56.836 | 120.270 | 70.586 | 1.00 | 44.57 | D |
| ATOM | 3727 | C | MET | D | 101 | 57.322 | 114.965 | 70.393 | 1.00 | 38.30 | D |
| ATOM | 3728 | O | MET | D | 101 | 57.567 | 114.828 | 69.192 | 1.00 | 38.66 | D |
| ATOM | 3729 | N | ILE | D | 102 | 56.113 | 114.725 | 70.934 | 1.00 | 36.58 | D |
| ATOM | 3730 | CA | ILE | D | 102 | 54.984 | 114.425 | 70.077 | 1.00 | 35.93 | D |
| ATOM | 3731 | CB | ILE | D | 102 | 54.156 | 113.269 | 70.521 | 1.00 | 35.38 | D |
| ATOM | 3732 | CG2 | ILE | D | 102 | 53.008 | 113.111 | 69.543 | 1.00 | 35.07 | D |
| ATOM | 3733 | CG1 | ILE | D | 102 | 54.989 | 112.005 | 70.519 | 1.00 | 34.18 | D |
| ATOM | 3734 | CD1 | ILE | D | 102 | 54.196 | 110.762 | 70.679 | 1.00 | 35.31 | D |
| ATOM | 3735 | C | ILE | D | 102 | 54.108 | 115.634 | 70.113 | 1.00 | 35.88 | D |
| ATOM | 3736 | O | ILE | D | 102 | 53.423 | 115.862 | 71.081 | 1.00 | 37.93 | D |
| ATOM | 3737 | N | LEU | D | 103 | 54.135 | 116.417 | 69.048 | 1.00 | 36.79 | D |
| ATOM | 3738 | CA | LEU | D | 103 | 53.367 | 117.664 | 68.925 | 1.00 | 36.91 | D |
| ATOM | 3739 | CB | LEU | D | 103 | 54.273 | 118.700 | 68.253 | 1.00 | 36.47 | D |
| ATOM | 3740 | CG | LEU | D | 103 | 53.764 | 120.089 | 67.945 | 1.00 | 36.59 | D |
| ATOM | 3741 | CD1 | LEU | D | 103 | 53.300 | 120.673 | 69.197 | 1.00 | 36.58 | D |
| ATOM | 3742 | CD2 | LEU | D | 103 | 54.880 | 120.939 | 67.346 | 1.00 | 35.48 | D |
| ATOM | 3743 | C | LEU | D | 103 | 52.035 | 117.501 | 68.131 | 1.00 | 36.66 | D |
| ATOM | 3744 | O | LEU | D | 103 | 52.054 | 117.250 | 66.907 | 1.00 | 36.75 | D |
| ATOM | 3745 | N | ASN | D | 104 | 50.889 | 117.659 | 68.807 | 1.00 | 35.94 | D |
| ATOM | 3746 | CA | ASN | D | 104 | 49.584 | 117.489 | 68.172 | 1.00 | 34.59 | D |
| ATOM | 3747 | CB | ASN | D | 104 | 49.247 | 118.622 | 67.185 | 1.00 | 34.05 | D |
| ATOM | 3748 | CG | ASN | D | 104 | 49.043 | 119.948 | 67.864 | 1.00 | 35.09 | D |
| ATOM | 3749 | OD1 | ASN | D | 104 | 48.818 | 120.016 | 69.062 | 1.00 | 37.53 | D |
| ATOM | 3750 | ND2 | ASN | D | 104 | 49.128 | 121.022 | 67.099 | 1.00 | 35.92 | D |
| ATOM | 3751 | C | ASN | D | 104 | 49.513 | 116.132 | 67.440 | 1.00 | 35.40 | D |
| ATOM | 3752 | O | ASN | D | 104 | 49.034 | 116.007 | 66.298 | 1.00 | 33.24 | D |
| ATOM | 3753 | N | GLY | D | 105 | 49.986 | 115.095 | 68.112 | 1.00 | 35.85 | D |
| ATOM | 3754 | CA | GLY | D | 105 | 49.913 | 113.779 | 67.528 | 1.00 | 37.38 | D |
| ATOM | 3755 | C | GLY | D | 105 | 50.935 | 113.406 | 66.460 | 1.00 | 39.75 | D |
| ATOM | 3756 | O | GLY | D | 105 | 50.785 | 112.345 | 65.806 | 1.00 | 40.32 | D |
| ATOM | 3757 | N | VAL | D | 106 | 51.946 | 114.236 | 66.213 | 1.00 | 38.67 | D |
| ATOM | 3758 | CA | VAL | D | 106 | 52.904 | 113.834 | 65.205 | 1.00 | 38.63 | D |
| ATOM | 3759 | CB | VAL | D | 106 | 52.752 | 114.681 | 63.909 | 1.00 | 39.72 | D |
| ATOM | 3760 | CG1 | VAL | D | 106 | 51.665 | 115.703 | 64.080 | 1.00 | 36.79 | D |
| ATOM | 3761 | CG2 | VAL | D | 106 | 54.094 | 115.280 | 63.498 | 1.00 | 38.14 | D |
| ATOM | 3762 | C | VAL | D | 106 | 54.305 | 113.912 | 65.782 | 1.00 | 38.76 | D |
| ATOM | 3763 | O | VAL | D | 106 | 54.650 | 114.855 | 66.449 | 1.00 | 39.58 | D |
| ATOM | 3764 | N | CYS | D | 107 | 55.105 | 112.886 | 65.577 | 1.00 | 38.67 | D |
| ATOM | 3765 | CA | CYS | D | 107 | 56.449 | 112.897 | 66.114 | 1.00 | 38.69 | D |
| ATOM | 3766 | CB | CYS | D | 107 | 57.050 | 111.505 | 66.048 | 1.00 | 39.37 | D |
| ATOM | 3767 | SG | CYS | D | 107 | 56.158 | 110.233 | 66.989 | 1.00 | 39.50 | D |
| ATOM | 3768 | C | CYS | D | 107 | 57.362 | 113.875 | 65.378 | 1.00 | 39.37 | D |
| ATOM | 3769 | O | CYS | D | 107 | 57.504 | 113.827 | 64.151 | 1.00 | 39.17 | D |
| ATOM | 3770 | N | VAL | D | 108 | 57.973 | 114.784 | 66.129 | 1.00 | 39.24 | D |
| ATOM | 3771 | CA | VAL | D | 108 | 58.838 | 115.761 | 65.518 | 1.00 | 39.79 | D |
| ATOM | 3772 | CB | VAL | D | 108 | 58.142 | 117.138 | 65.303 | 1.00 | 40.13 | D |
| ATOM | 3773 | CG1 | VAL | D | 108 | 56.870 | 116.991 | 64.454 | 1.00 | 39.67 | D |
| ATOM | 3774 | CG2 | VAL | D | 108 | 57.822 | 117.758 | 66.625 | 1.00 | 39.84 | D |
| ATOM | 3775 | C | VAL | D | 108 | 60.023 | 115.980 | 66.402 | 1.00 | 40.21 | D |
| ATOM | 3776 | O | VAL | D | 108 | 60.077 | 115.459 | 67.508 | 1.00 | 40.07 | D |
| ATOM | 3777 | N | ILE | D | 109 | 60.986 | 116.721 | 65.868 | 1.00 | 41.20 | D |
| ATOM | 3778 | CA | ILE | D | 109 | 62.197 | 117.070 | 66.576 | 1.00 | 42.21 | D |
| ATOM | 3779 | CB | ILE | D | 109 | 63.419 | 116.291 | 66.037 | 1.00 | 43.31 | D |
| ATOM | 3780 | CG2 | ILE | D | 109 | 64.683 | 116.733 | 66.743 | 1.00 | 43.03 | D |
| ATOM | 3781 | CG1 | ILE | D | 109 | 63.213 | 114.779 | 66.277 | 1.00 | 44.19 | D |
| ATOM | 3782 | CD1 | ILE | D | 109 | 64.198 | 113.889 | 65.534 | 1.00 | 44.87 | D |
| ATOM | 3783 | C | ILE | D | 109 | 62.363 | 118.547 | 66.324 | 1.00 | 42.08 | D |
| ATOM | 3784 | O | ILE | D | 109 | 62.253 | 119.026 | 65.191 | 1.00 | 41.69 | D |
| ATOM | 3785 | N | TRP | D | 110 | 62.568 | 119.261 | 67.419 | 1.00 | 43.02 | D |
| ATOM | 3786 | CA | TRP | D | 110 | 62.766 | 120.700 | 67.414 | 1.00 | 44.70 | D |
| ATOM | 3787 | CB | TRP | D | 110 | 62.217 | 121.297 | 68.709 | 1.00 | 45.36 | D |
| ATOM | 3788 | CG | TRP | D | 110 | 62.324 | 122.797 | 68.765 | 1.00 | 46.52 | D |
| ATOM | 3789 | CD2 | TRP | D | 110 | 63.462 | 123.574 | 69.173 | 1.00 | 46.82 | D |
| ATOM | 3790 | CE2 | TRP | D | 110 | 63.131 | 124.933 | 68.975 | 1.00 | 47.69 | D |
| ATOM | 3791 | CE3 | TRP | D | 110 | 64.729 | 123.255 | 69.675 | 1.00 | 46.95 | D |
| ATOM | 3792 | CD1 | TRP | D | 110 | 61.387 | 123.687 | 68.357 | 1.00 | 46.85 | D |
| ATOM | 3793 | NE1 | TRP | D | 110 | 61.856 | 124.969 | 68.477 | 1.00 | 47.30 | D |
| ATOM | 3794 | CZ2 | TRP | D | 110 | 64.030 | 125.994 | 69.266 | 1.00 | 48.53 | D |
| ATOM | 3795 | CZ3 | TRP | D | 110 | 65.632 | 124.305 | 69.966 | 1.00 | 48.31 | D |
| ATOM | 3796 | CH2 | TRP | D | 110 | 65.273 | 125.661 | 69.758 | 1.00 | 48.04 | D |
| ATOM | 3797 | C | TRP | D | 110 | 64.270 | 120.922 | 67.362 | 1.00 | 45.41 | D |
| ATOM | 3798 | O | TRP | D | 110 | 65.019 | 120.405 | 68.210 | 1.00 | 44.36 | D |
| ATOM | 3799 | N | LYS | D | 111 | 64.713 | 121.673 | 66.363 | 1.00 | 47.07 | D |
| ATOM | 3800 | CA | LYS | D | 111 | 66.148 | 121.952 | 66.205 | 1.00 | 48.48 | D |
| ATOM | 3801 | CB | LYS | D | 111 | 66.709 | 121.271 | 64.973 | 1.00 | 50.21 | D |
| ATOM | 3802 | CG | LYS | D | 111 | 66.731 | 119.746 | 64.954 | 1.00 | 53.59 | D |
| ATOM | 3803 | CD | LYS | D | 111 | 67.299 | 119.352 | 63.586 | 1.00 | 57.44 | D |
| ATOM | 3804 | CE | LYS | D | 111 | 67.525 | 117.866 | 63.392 | 1.00 | 59.32 | D |
| ATOM | 3805 | NZ | LYS | D | 111 | 68.329 | 117.643 | 62.138 | 1.00 | 61.56 | D |
| ATOM | 3806 | C | LYS | D | 111 | 66.364 | 123.424 | 66.024 | 1.00 | 48.33 | D |
| ATOM | 3807 | O | LYS | D | 111 | 65.804 | 124.028 | 65.112 | 1.00 | 47.29 | D |
| ATOM | 3808 | N | GLY | D | 112 | 67.185 | 123.999 | 66.887 | 1.00 | 49.41 | D |
| ATOM | 3809 | CA | GLY | D | 112 | 67.479 | 125.417 | 66.800 | 1.00 | 50.62 | D |
| ATOM | 3810 | C | GLY | D | 112 | 68.730 | 125.773 | 67.582 | 1.00 | 51.94 | D |
| ATOM | 3811 | O | GLY | D | 112 | 69.293 | 124.952 | 68.329 | 1.00 | 52.74 | D |
| ATOM | 3812 | N | TRP | D | 113 | 69.181 | 127.008 | 67.409 | 1.00 | 52.98 | D |
| ATOM | 3813 | CA | TRP | D | 113 | 70.361 | 127.496 | 68.114 | 1.00 | 53.38 | D |
| ATOM | 3814 | CB | TRP | D | 113 | 71.551 | 127.736 | 67.157 | 1.00 | 51.47 | D |
| ATOM | 3815 | CG | TRP | D | 113 | 71.300 | 128.692 | 66.007 | 1.00 | 49.44 | D |
| ATOM | 3816 | CD2 | TRP | D | 113 | 71.342 | 130.125 | 66.057 | 1.00 | 49.14 | D |
| ATOM | 3817 | CE2 | TRP | D | 113 | 71.047 | 130.605 | 64.754 | 1.00 | 49.25 | D |
| ATOM | 3818 | CE3 | TRP | D | 113 | 71.601 | 131.057 | 67.077 | 1.00 | 48.41 | D |
| ATOM | 3819 | CD1 | TRP | D | 113 | 70.985 | 128.365 | 64.709 | 1.00 | 49.67 | D |
| ATOM | 3820 | NE1 | TRP | D | 113 | 70.832 | 129.515 | 63.945 | 1.00 | 49.75 | D |
| ATOM | 3821 | CZ2 | TRP | D | 113 | 71.008 | 131.968 | 64.454 | 1.00 | 48.76 | D |
| ATOM | 3822 | CZ3 | TRP | D | 113 | 71.560 | 132.419 | 66.773 | 1.00 | 46.84 | D |
| ATOM | 3823 | CH2 | TRP | D | 113 | 71.267 | 132.861 | 65.482 | 1.00 | 47.81 | D |
| ATOM | 3824 | C | TRP | D | 113 | 69.980 | 128.798 | 68.797 | 1.00 | 55.10 | D |
| ATOM | 3825 | O | TRP | D | 113 | 69.026 | 129.493 | 68.388 | 1.00 | 54.83 | D |
| ATOM | 3826 | N | ILE | D | 114 | 70.736 | 129.121 | 69.834 | 1.00 | 56.77 | D |
| ATOM | 3827 | CA | ILE | D | 114 | 70.495 | 130.319 | 70.577 | 1.00 | 59.23 | D |
| ATOM | 3828 | CB | ILE | D | 114 | 69.638 | 129.978 | 71.828 | 1.00 | 59.05 | D |
| ATOM | 3829 | CG2 | ILE | D | 114 | 69.869 | 130.968 | 72.964 | 1.00 | 57.50 | D |
| ATOM | 3830 | CG1 | ILE | D | 114 | 68.171 | 129.960 | 71.416 | 1.00 | 59.07 | D |
| ATOM | 3831 | CD1 | ILE | D | 114 | 67.342 | 129.267 | 72.424 | 1.00 | 60.09 | D |
| ATOM | 3832 | C | ILE | D | 114 | 71.805 | 130.998 | 70.951 | 1.00 | 61.18 | D |
| ATOM | 3833 | O | ILE | D | 114 | 72.760 | 130.340 | 71.376 | 1.00 | 61.11 | D |
| ATOM | 3834 | N | ASP | D | 115 | 71.824 | 132.319 | 70.758 | 1.00 | 62.88 | D |
| ATOM | 3835 | CA | ASP | D | 115 | 72.945 | 133.182 | 71.089 | 1.00 | 64.33 | D |
| ATOM | 3836 | CB | ASP | D | 115 | 72.649 | 134.555 | 70.531 | 1.00 | 65.15 | D |
| ATOM | 3837 | CG | ASP | D | 115 | 73.703 | 135.599 | 70.899 | 1.00 | 66.77 | D |
| ATOM | 3838 | OD1 | ASP | D | 115 | 74.080 | 135.727 | 72.106 | 1.00 | 64.98 | D |
| ATOM | 3839 | OD2 | ASP | D | 115 | 74.122 | 136.314 | 69.937 | 1.00 | 68.08 | D |
| ATOM | 3840 | C | ASP | D | 115 | 72.959 | 133.266 | 72.606 | 1.00 | 65.49 | D |
| ATOM | 3841 | O | ASP | D | 115 | 72.056 | 133.892 | 73.177 | 1.00 | 66.63 | D |
| ATOM | 3842 | N | LEU | D | 116 | 73.952 | 132.672 | 73.272 | 1.00 | 65.82 | D |
| ATOM | 3843 | CA | LEU | D | 116 | 73.995 | 132.713 | 74.736 | 1.00 | 66.06 | D |
| ATOM | 3844 | CB | LEU | D | 116 | 75.183 | 131.910 | 75.227 | 1.00 | 66.26 | D |
| ATOM | 3845 | CG | LEU | D | 116 | 75.210 | 130.463 | 74.725 | 1.00 | 67.03 | D |
| ATOM | 3846 | CD1 | LEU | D | 116 | 76.645 | 129.972 | 74.667 | 1.00 | 66.30 | D |
| ATOM | 3847 | CD2 | LEU | D | 116 | 74.369 | 129.582 | 75.631 | 1.00 | 65.55 | D |
| ATOM | 3848 | C | LEU | D | 116 | 74.034 | 134.134 | 75.336 | 1.00 | 66.81 | D |
| ATOM | 3849 | O | LEU | D | 116 | 73.951 | 134.317 | 76.551 | 1.00 | 65.90 | D |
| ATOM | 3850 | N | GLN | D | 117 | 74.138 | 135.142 | 74.480 | 1.00 | 67.97 | D |
| ATOM | 3851 | CA | GLN | D | 117 | 74.185 | 136.509 | 74.954 | 1.00 | 69.40 | D |
| ATOM | 3852 | CB | GLN | D | 117 | 75.396 | 137.227 | 74.324 | 1.00 | 72.09 | D |
| ATOM | 3853 | CG | GLN | D | 117 | 75.783 | 138.546 | 75.006 | 1.00 | 75.66 | D |
| ATOM | 3854 | CD | GLN | D | 117 | 75.093 | 139.767 | 74.374 | 1.00 | 77.82 | D |
| ATOM | 3855 | OE1 | GLN | D | 117 | 75.684 | 140.452 | 73.523 | 1.00 | 78.95 | D |
| ATOM | 3856 | NE2 | GLN | D | 117 | 73.834 | 140.036 | 74.778 | 1.00 | 77.92 | D |
| ATOM | 3857 | C | GLN | D | 117 | 72.873 | 137.258 | 74.657 | 1.00 | 69.01 | D |
| ATOM | 3858 | O | GLN | D | 117 | 72.321 | 137.900 | 75.544 | 1.00 | 69.68 | D |
| ATOM | 3859 | N | ARG | D | 118 | 72.361 | 137.202 | 73.431 | 1.00 | 67.58 | D |
| ATOM | 3860 | CA | ARG | D | 118 | 71.096 | 137.892 | 73.155 | 1.00 | 66.55 | D |
| ATOM | 3861 | CB | ARG | D | 118 | 70.899 | 138.060 | 71.655 | 1.00 | 67.41 | D |
| ATOM | 3862 | CG | ARG | D | 118 | 72.154 | 138.378 | 70.871 | 1.00 | 68.99 | D |
| ATOM | 3863 | CD | ARG | D | 118 | 71.787 | 138.632 | 69.420 | 1.00 | 69.45 | D |
| ATOM | 3864 | NE | ARG | D | 118 | 70.734 | 139.638 | 69.388 | 1.00 | 70.64 | D |
| ATOM | 3865 | CZ | ARG | D | 118 | 70.129 | 140.073 | 68.289 | 1.00 | 71.88 | D |
| ATOM | 3866 | NH1 | ARG | D | 118 | 70.474 | 139.596 | 67.094 | 1.00 | 72.20 | D |
| ATOM | 3867 | NH2 | ARG | D | 118 | 69.159 | 140.980 | 68.392 | 1.00 | 71.77 | D |
| ATOM | 3868 | C | ARG | D | 118 | 69.907 | 137.075 | 73.712 | 1.00 | 65.76 | D |
| ATOM | 3869 | O | ARG | D | 118 | 68.883 | 137.627 | 74.133 | 1.00 | 65.16 | D |
| ATOM | 3870 | N | LEU | D | 119 | 70.067 | 135.750 | 73.730 | 1.00 | 64.24 | D |
| ATOM | 3871 | CA | LEU | D | 119 | 69.014 | 134.841 | 74.188 | 1.00 | 62.55 | D |
| ATOM | 3872 | CB | LEU | D | 119 | 68.456 | 135.301 | 75.541 | 1.00 | 60.51 | D |
| ATOM | 3873 | CG | LEU | D | 119 | 69.428 | 135.069 | 76.695 | 1.00 | 59.42 | D |
| ATOM | 3874 | CD1 | LEU | D | 119 | 68.740 | 135.396 | 77.978 | 1.00 | 57.96 | D |
| ATOM | 3875 | CD2 | LEU | D | 119 | 69.914 | 133.632 | 76.710 | 1.00 | 58.74 | D |
| ATOM | 3876 | C | LEU | D | 119 | 67.870 | 134.639 | 73.147 | 1.00 | 62.42 | D |
| ATOM | 3877 | O | LEU | D | 119 | 66.728 | 134.328 | 73.514 | 1.00 | 62.62 | D |
| ATOM | 3878 | N | ASP | D | 120 | 68.189 | 134.820 | 71.858 | 1.00 | 61.13 | D |
| ATOM | 3879 | CA | ASP | D | 120 | 67.242 | 134.598 | 70.764 | 1.00 | 60.12 | D |
| ATOM | 3880 | CB | ASP | D | 120 | 66.717 | 135.915 | 70.198 | 1.00 | 60.18 | D |
| ATOM | 3881 | CG | ASP | D | 120 | 67.795 | 136.774 | 69.606 | 1.00 | 60.92 | D |
| ATOM | 3882 | OD1 | ASP | D | 120 | 68.983 | 136.400 | 69.691 | 1.00 | 62.58 | D |
| ATOM | 3883 | OD2 | ASP | D | 120 | 67.446 | 137.835 | 69.053 | 1.00 | 60.11 | D |
| ATOM | 3884 | C | ASP | D | 120 | 67.948 | 133.781 | 69.673 | 1.00 | 59.64 | D |
| ATOM | 3885 | O | ASP | D | 120 | 69.088 | 133.363 | 69.858 | 1.00 | 60.07 | D |
| ATOM | 3886 | N | GLY | D | 121 | 67.295 | 133.518 | 68.550 | 1.00 | 57.93 | D |
| ATOM | 3887 | CA | GLY | D | 121 | 67.981 | 132.725 | 67.564 | 1.00 | 56.32 | D |
| ATOM | 3888 | C | GLY | D | 121 | 67.163 | 132.281 | 66.386 | 1.00 | 56.51 | D |
| ATOM | 3889 | O | GLY | D | 121 | 66.390 | 133.052 | 65.808 | 1.00 | 56.18 | D |
| ATOM | 3890 | N | MET | D | 122 | 67.378 | 131.028 | 66.002 | 1.00 | 56.17 | D |
| ATOM | 3891 | CA | MET | D | 122 | 66.649 | 130.432 | 64.896 | 1.00 | 56.11 | D |
| ATOM | 3892 | CB | MET | D | 122 | 67.444 | 130.534 | 63.592 | 1.00 | 56.16 | D |
| ATOM | 3893 | CG | MET | D | 122 | 67.753 | 131.948 | 63.150 | 1.00 | 55.48 | D |
| ATOM | 3894 | SD | MET | D | 122 | 66.293 | 132.857 | 62.749 | 1.00 | 56.32 | D |
| ATOM | 3895 | CE | MET | D | 122 | 66.127 | 132.533 | 60.989 | 1.00 | 55.29 | D |
| ATOM | 3896 | C | MET | D | 122 | 66.389 | 128.968 | 65.221 | 1.00 | 56.12 | D |
| ATOM | 3897 | O | MET | D | 122 | 67.128 | 128.331 | 65.975 | 1.00 | 56.24 | D |
| ATOM | 3898 | N | GLY | D | 123 | 65.315 | 128.442 | 64.658 | 1.00 | 55.47 | D |
| ATOM | 3899 | CA | GLY | D | 123 | 64.982 | 127.053 | 64.887 | 1.00 | 53.89 | D |
| ATOM | 3900 | C | GLY | D | 123 | 63.785 | 126.652 | 64.047 | 1.00 | 52.30 | D |
| ATOM | 3901 | O | GLY | D | 123 | 63.153 | 127.477 | 63.403 | 1.00 | 52.34 | D |
| ATOM | 3902 | N | CYS | D | 124 | 63.471 | 125.378 | 64.044 | 1.00 | 50.70 | D |
| ATOM | 3903 | CA | CYS | D | 124 | 62.348 | 124.955 | 63.274 | 1.00 | 50.30 | D |
| ATOM | 3904 | CB | CYS | D | 124 | 62.700 | 124.918 | 61.791 | 1.00 | 50.59 | D |
| ATOM | 3905 | SG | CYS | D | 124 | 63.862 | 123.596 | 61.432 | 1.00 | 49.58 | D |
| ATOM | 3906 | C | CYS | D | 124 | 61.988 | 123.579 | 63.756 | 1.00 | 50.04 | D |
| ATOM | 3907 | O | CYS | D | 124 | 62.656 | 122.993 | 64.611 | 1.00 | 50.95 | D |
| ATOM | 3908 | N | LEU | D | 125 | 60.921 | 123.069 | 63.188 | 1.00 | 49.42 | D |
| ATOM | 3909 | CA | LEU | D | 125 | 60.407 | 121.788 | 63.549 | 1.00 | 49.28 | D |
| ATOM | 3910 | CB | LEU | D | 125 | 58.890 | 121.921 | 63.687 | 1.00 | 48.02 | D |
| ATOM | 3911 | CG | LEU | D | 125 | 58.249 | 121.678 | 65.051 | 1.00 | 46.05 | D |
| ATOM | 3912 | CD1 | LEU | D | 125 | 59.031 | 122.326 | 66.151 | 1.00 | 44.89 | D |
| ATOM | 3913 | CD2 | LEU | D | 125 | 56.844 | 122.187 | 65.014 | 1.00 | 44.10 | D |
| ATOM | 3914 | C | LEU | D | 125 | 60.778 | 120.899 | 62.403 | 1.00 | 49.88 | D |
| ATOM | 3915 | O | LEU | D | 125 | 60.735 | 121.331 | 61.263 | 1.00 | 48.86 | D |
| ATOM | 3916 | N | GLU | D | 126 | 61.129 | 119.656 | 62.712 | 1.00 | 51.42 | D |
| ATOM | 3917 | CA | GLU | D | 126 | 61.540 | 118.668 | 61.700 | 1.00 | 52.92 | D |
| ATOM | 3918 | CB | GLU | D | 126 | 63.032 | 118.398 | 61.903 | 1.00 | 53.96 | D |
| ATOM | 3919 | CG | GLU | D | 126 | 63.851 | 118.299 | 60.659 | 1.00 | 57.77 | D |
| ATOM | 3920 | CD | GLU | D | 126 | 65.311 | 117.962 | 60.982 | 1.00 | 60.55 | D |
| ATOM | 3921 | OE1 | GLU | D | 126 | 65.576 | 116.882 | 61.582 | 1.00 | 61.17 | D |
| ATOM | 3922 | OE2 | GLU | D | 126 | 66.196 | 118.781 | 60.635 | 1.00 | 61.86 | D |
| ATOM | 3923 | C | GLU | D | 126 | 60.737 | 117.362 | 61.887 | 1.00 | 52.54 | D |
| ATOM | 3924 | O | GLU | D | 126 | 60.590 | 116.878 | 63.026 | 1.00 | 53.05 | D |
| ATOM | 3925 | N | PHE | D | 127 | 60.209 | 116.777 | 60.814 | 1.00 | 51.50 | D |
| ATOM | 3926 | CA | PHE | D | 127 | 59.457 | 115.538 | 60.995 | 1.00 | 50.28 | D |
| ATOM | 3927 | CB | PHE | D | 127 | 58.793 | 115.098 | 59.702 | 1.00 | 49.80 | D |
| ATOM | 3928 | CG | PHE | D | 127 | 57.949 | 113.838 | 59.833 | 1.00 | 51.81 | D |
| ATOM | 3929 | CD1 | PHE | D | 127 | 56.790 | 113.825 | 60.628 | 1.00 | 52.34 | D |
| ATOM | 3930 | CD2 | PHE | D | 127 | 58.256 | 112.692 | 59.109 | 1.00 | 51.72 | D |
| ATOM | 3931 | CE1 | PHE | D | 127 | 55.938 | 112.678 | 60.685 | 1.00 | 52.37 | D |
| ATOM | 3932 | CE2 | PHE | D | 127 | 57.422 | 111.564 | 59.164 | 1.00 | 53.14 | D |
| ATOM | 3933 | CZ | PHE | D | 127 | 56.249 | 111.566 | 59.962 | 1.00 | 52.65 | D |
| ATOM | 3934 | C | PHE | D | 127 | 60.403 | 114.450 | 61.479 | 1.00 | 49.74 | D |
| ATOM | 3935 | O | PHE | D | 127 | 61.443 | 114.255 | 60.898 | 1.00 | 48.89 | D |
| ATOM | 3936 | N | ASP | D | 128 | 60.063 | 113.765 | 62.563 | 1.00 | 49.96 | D |
| ATOM | 3937 | CA | ASP | D | 128 | 60.915 | 112.707 | 63.048 | 1.00 | 51.36 | D |
| ATOM | 3938 | CB | ASP | D | 128 | 60.904 | 112.706 | 64.551 | 1.00 | 50.76 | D |
| ATOM | 3939 | CG | ASP | D | 128 | 61.842 | 111.697 | 65.118 | 1.00 | 51.02 | D |
| ATOM | 3940 | OD1 | ASP | D | 128 | 62.197 | 110.759 | 64.352 | 1.00 | 50.60 | D |
| ATOM | 3941 | OD2 | ASP | D | 128 | 62.206 | 111.838 | 66.313 | 1.00 | 50.83 | D |
| ATOM | 3942 | C | ASP | D | 128 | 60.443 | 111.346 | 62.524 | 1.00 | 52.78 | D |
| ATOM | 3943 | O | ASP | D | 128 | 59.830 | 110.577 | 63.250 | 1.00 | 52.61 | D |
| ATOM | 3944 | N | GLU | D | 129 | 60.762 | 111.033 | 61.270 | 1.00 | 54.48 | D |
| ATOM | 3945 | CA | GLU | D | 129 | 60.284 | 109.787 | 60.694 | 1.00 | 56.11 | D |
| ATOM | 3946 | CB | GLU | D | 129 | 60.655 | 109.631 | 59.192 | 1.00 | 56.77 | D |
| ATOM | 3947 | CG | GLU | D | 129 | 62.110 | 109.268 | 58.776 | 1.00 | 58.52 | D |
| ATOM | 3948 | CD | GLU | D | 129 | 62.522 | 107.769 | 59.006 | 1.00 | 60.92 | D |
| ATOM | 3949 | OE1 | GLU | D | 129 | 61.647 | 106.846 | 58.957 | 1.00 | 59.33 | D |
| ATOM | 3950 | OE2 | GLU | D | 129 | 63.756 | 107.517 | 59.216 | 1.00 | 61.86 | D |
| ATOM | 3951 | C | GLU | D | 129 | 60.687 | 108.560 | 61.462 | 1.00 | 57.36 | D |
| ATOM | 3952 | O | GLU | D | 129 | 59.920 | 107.596 | 61.495 | 1.00 | 55.86 | D |
| ATOM | 3953 | N | GLU | D | 130 | 61.859 | 108.570 | 62.101 | 1.00 | 59.40 | D |
| ATOM | 3954 | CA | GLU | D | 130 | 62.253 | 107.362 | 62.837 | 1.00 | 61.36 | D |
| ATOM | 3955 | CB | GLU | D | 130 | 63.630 | 107.545 | 63.514 | 1.00 | 64.81 | D |
| ATOM | 3956 | CG | GLU | D | 130 | 64.372 | 106.216 | 63.849 | 1.00 | 69.16 | D |
| ATOM | 3957 | CD | GLU | D | 130 | 65.888 | 106.410 | 64.172 | 1.00 | 72.56 | D |
| ATOM | 3958 | OE1 | GLU | D | 130 | 66.239 | 106.837 | 65.318 | 1.00 | 73.36 | D |
| ATOM | 3959 | OE2 | GLU | D | 130 | 66.729 | 106.138 | 63.265 | 1.00 | 72.98 | D |
| ATOM | 3960 | C | GLU | D | 130 | 61.143 | 107.035 | 63.862 | 1.00 | 61.09 | D |
| ATOM | 3961 | O | GLU | D | 130 | 60.506 | 105.973 | 63.751 | 1.00 | 60.57 | D |
| ATOM | 3962 | N | ARG | D | 131 | 60.891 | 107.966 | 64.805 | 1.00 | 60.99 | D |
| ATOM | 3963 | CA | ARG | D | 131 | 59.872 | 107.857 | 65.874 | 1.00 | 60.38 | D |
| ATOM | 3964 | CB | ARG | D | 131 | 59.847 | 109.138 | 66.701 | 1.00 | 60.16 | D |
| ATOM | 3965 | CG | ARG | D | 131 | 61.046 | 109.307 | 67.568 | 1.00 | 63.20 | D |
| ATOM | 3966 | CD | ARG | D | 131 | 60.878 | 108.486 | 68.823 | 1.00 | 66.58 | D |
| ATOM | 3967 | NE | ARG | D | 131 | 59.705 | 108.968 | 69.568 | 1.00 | 70.87 | D |
| ATOM | 3968 | CZ | ARG | D | 131 | 59.174 | 108.399 | 70.656 | 1.00 | 71.58 | D |
| ATOM | 3969 | NH1 | ARG | D | 131 | 59.702 | 107.285 | 71.177 | 1.00 | 70.33 | D |
| ATOM | 3970 | NH2 | ARG | D | 131 | 58.095 | 108.960 | 71.213 | 1.00 | 71.56 | D |
| ATOM | 3971 | C | ARG | D | 131 | 58.465 | 107.585 | 65.361 | 1.00 | 59.96 | D |
| ATOM | 3972 | O | ARG | D | 131 | 57.784 | 106.669 | 65.815 | 1.00 | 58.70 | D |
| ATOM | 3973 | N | ALA | D | 132 | 58.033 | 108.396 | 64.410 | 1.00 | 60.38 | D |
| ATOM | 3974 | CA | ALA | D | 132 | 56.700 | 108.244 | 63.858 | 1.00 | 62.37 | D |
| ATOM | 3975 | CB | ALA | D | 132 | 56.510 | 109.200 | 62.655 | 1.00 | 60.88 | D |
| ATOM | 3976 | C | ALA | D | 132 | 56.436 | 106.788 | 63.456 | 1.00 | 63.78 | D |
| ATOM | 3977 | O | ALA | D | 132 | 55.320 | 106.273 | 63.607 | 1.00 | 64.02 | D |
| ATOM | 3978 | N | GLN | D | 133 | 57.479 | 106.118 | 62.979 | 1.00 | 65.89 | D |
| ATOM | 3979 | CA | GLN | D | 133 | 57.374 | 104.735 | 62.537 | 1.00 | 67.56 | D |
| ATOM | 3980 | CB | GLN | D | 133 | 58.566 | 104.440 | 61.566 | 1.00 | 68.87 | D |
| ATOM | 3981 | CG | GLN | D | 133 | 59.366 | 103.149 | 61.749 | 1.00 | 72.49 | D |
| ATOM | 3982 | CD | GLN | D | 133 | 58.729 | 101.916 | 61.066 | 1.00 | 74.79 | D |
| ATOM | 3983 | OE1 | GLN | D | 133 | 59.303 | 100.806 | 61.099 | 1.00 | 74.99 | D |
| ATOM | 3984 | NE2 | GLN | D | 133 | 57.545 | 102.105 | 60.447 | 1.00 | 74.67 | D |
| ATOM | 3985 | C | GLN | D | 133 | 57.246 | 103.780 | 63.758 | 1.00 | 67.53 | D |
| ATOM | 3986 | O | GLN | D | 133 | 56.570 | 102.755 | 63.669 | 1.00 | 67.90 | D |
| ATOM | 3987 | N | GLN | D | 134 | 57.806 | 104.139 | 64.913 | 1.00 | 67.18 | D |
| ATOM | 3988 | CA | GLN | D | 134 | 57.697 | 103.278 | 66.091 | 1.00 | 67.12 | D |
| ATOM | 3989 | CB | GLN | D | 134 | 58.862 | 103.528 | 67.033 | 1.00 | 67.84 | D |
| ATOM | 3990 | CG | GLN | D | 134 | 60.220 | 103.343 | 66.389 | 1.00 | 70.77 | D |
| ATOM | 3991 | CD | GLN | D | 134 | 61.324 | 104.091 | 67.152 | 1.00 | 73.37 | D |
| ATOM | 3992 | OE1 | GLN | D | 134 | 61.292 | 105.323 | 67.283 | 1.00 | 74.62 | D |
| ATOM | 3993 | NE2 | GLN | D | 134 | 62.298 | 103.349 | 67.666 | 1.00 | 74.45 | D |
| ATOM | 3994 | C | GLN | D | 134 | 56.400 | 103.482 | 66.866 | 1.00 | 66.84 | D |
| ATOM | 3995 | O | GLN | D | 134 | 56.050 | 102.647 | 67.675 | 1.00 | 67.27 | D |
| ATOM | 3996 | N | GLU | D | 135 | 55.683 | 104.574 | 66.614 | 1.00 | 66.69 | D |
| ATOM | 3997 | CA | GLU | D | 135 | 54.442 | 104.882 | 67.346 | 1.00 | 66.72 | D |
| ATOM | 3998 | CB | GLU | D | 135 | 54.127 | 106.378 | 67.246 | 1.00 | 65.38 | D |
| ATOM | 3999 | CG | GLU | D | 135 | 54.918 | 107.263 | 68.197 | 1.00 | 63.42 | D |
| ATOM | 4000 | CD | GLU | D | 135 | 54.729 | 106.904 | 69.670 | 1.00 | 62.92 | D |
| ATOM | 4001 | OE1 | GLU | D | 135 | 53.632 | 106.438 | 70.058 | 1.00 | 62.55 | D |
| ATOM | 4002 | OE2 | GLU | D | 135 | 55.682 | 107.116 | 70.450 | 1.00 | 62.44 | D |
| ATOM | 4003 | C | GLU | D | 135 | 53.155 | 104.116 | 66.993 | 1.00 | 67.81 | D |
| ATOM | 4004 | O | GLU | D | 135 | 53.066 | 103.533 | 65.881 | 1.00 | 68.17 | D |
| ATOM | 4005 | OXT | GLU | D | 135 | 52.223 | 104.146 | 67.846 | 1.00 | 68.02 | D |
| ATOM | 4006 | O5′ | GUA | E | 1 | 108.204 | 119.958 | 66.777 | 1.00 | 40.30 | E |
| ATOM | 4007 | N9 | GUA | E | 1 | 111.377 | 118.617 | 65.300 | 1.00 | 37.33 | E |
| ATOM | 4008 | C4 | GUA | E | 1 | 112.547 | 117.911 | 65.209 | 1.00 | 35.99 | E |
| ATOM | 4009 | N3 | GUA | E | 1 | 113.768 | 118.439 | 65.229 | 1.00 | 35.17 | E |
| ATOM | 4010 | C2 | GUA | E | 1 | 114.710 | 117.512 | 65.163 | 1.00 | 36.48 | E |
| ATOM | 4011 | N2 | GUA | E | 1 | 115.997 | 117.884 | 65.203 | 1.00 | 34.15 | E |
| ATOM | 4012 | N1 | GUA | E | 1 | 114.449 | 116.155 | 65.071 | 1.00 | 37.21 | E |
| ATOM | 4013 | C6 | GUA | E | 1 | 113.166 | 115.594 | 65.088 | 1.00 | 37.34 | E |
| ATOM | 4014 | O6 | GUA | E | 1 | 113.014 | 114.346 | 65.090 | 1.00 | 39.72 | E |
| ATOM | 4015 | C5 | GUA | E | 1 | 112.181 | 116.579 | 65.141 | 1.00 | 36.58 | E |
| ATOM | 4016 | N7 | GUA | E | 1 | 110.809 | 116.444 | 65.187 | 1.00 | 39.27 | E |
| ATOM | 4017 | C8 | GUA | E | 1 | 110.366 | 117.681 | 65.288 | 1.00 | 38.81 | E |
| ATOM | 4018 | C2′ | GUA | E | 1 | 110.149 | 120.789 | 64.594 | 1.00 | 34.59 | E |
| ATOM | 4019 | C5′ | GUA | E | 1 | 108.749 | 121.004 | 67.510 | 1.00 | 34.75 | E |
| ATOM | 4020 | C4′ | GUA | E | 1 | 110.039 | 121.486 | 66.882 | 1.00 | 36.71 | E |
| ATOM | 4021 | O4′ | GUA | E | 1 | 111.014 | 120.399 | 66.759 | 1.00 | 36.47 | E |
| ATOM | 4022 | C1′ | GUA | E | 1 | 111.246 | 120.070 | 65.395 | 1.00 | 35.89 | E |
| ATOM | 4023 | C3′ | GUA | E | 1 | 109.899 | 122.029 | 65.448 | 1.00 | 34.34 | E |
| ATOM | 4024 | O3′ | GUA | E | 1 | 110.895 | 123.068 | 65.351 | 1.00 | 33.29 | E |
| ATOM | 4025 | P | THY | E | 2 | 111.086 | 123.935 | 64.001 | 1.00 | 35.11 | E |
| ATOM | 4026 | O1P | THY | E | 2 | 110.173 | 123.371 | 62.984 | 1.00 | 35.85 | E |
| ATOM | 4027 | O2P | THY | E | 2 | 111.061 | 125.367 | 64.310 | 1.00 | 36.86 | E |
| ATOM | 4028 | O5′ | THY | E | 2 | 112.598 | 123.757 | 63.599 | 1.00 | 31.62 | E |
| ATOM | 4029 | N1 | THY | E | 2 | 114.311 | 119.406 | 61.824 | 1.00 | 33.66 | E |
| ATOM | 4030 | C6 | THY | E | 2 | 112.954 | 119.314 | 61.897 | 1.00 | 33.97 | E |
| ATOM | 4031 | C2 | THY | E | 2 | 115.089 | 118.313 | 61.673 | 1.00 | 34.31 | E |
| ATOM | 4032 | O2 | THY | E | 2 | 116.309 | 118.389 | 61.660 | 1.00 | 35.94 | E |
| ATOM | 4033 | N3 | THY | E | 2 | 114.415 | 117.126 | 61.558 | 1.00 | 34.58 | E |
| ATOM | 4034 | C4 | THY | E | 2 | 113.074 | 116.934 | 61.630 | 1.00 | 35.15 | E |
| ATOM | 4035 | O4 | THY | E | 2 | 112.621 | 115.785 | 61.568 | 1.00 | 37.29 | E |
| ATOM | 4036 | C5 | THY | E | 2 | 112.293 | 118.153 | 61.811 | 1.00 | 35.27 | E |
| ATOM | 4037 | C5A | THY | E | 2 | 110.798 | 118.072 | 61.901 | 1.00 | 31.88 | E |
| ATOM | 4038 | C2′ | THY | E | 2 | 114.521 | 121.819 | 60.997 | 1.00 | 33.91 | E |
| ATOM | 4039 | C5′ | THY | E | 2 | 113.168 | 122.508 | 63.750 | 1.00 | 34.75 | E |
| ATOM | 4040 | C4′ | THY | E | 2 | 114.582 | 122.516 | 63.263 | 1.00 | 34.85 | E |
| ATOM | 4041 | O4′ | THY | E | 2 | 114.919 | 121.112 | 63.182 | 1.00 | 35.81 | E |
| ATOM | 4042 | C1′ | THY | E | 2 | 115.022 | 120.681 | 61.878 | 1.00 | 34.33 | E |
| ATOM | 4043 | C3′ | THY | E | 2 | 114.771 | 123.058 | 61.853 | 1.00 | 35.17 | E |
| ATOM | 4044 | O3′ | THY | E | 2 | 116.090 | 123.641 | 61.776 | 1.00 | 35.62 | E |
| ATOM | 4045 | P | THY | E | 3 | 116.611 | 124.372 | 60.413 | 1.00 | 38.83 | E |
| ATOM | 4046 | O1P | THY | E | 3 | 115.533 | 124.376 | 59.396 | 1.00 | 37.67 | E |
| ATOM | 4047 | O2P | THY | E | 3 | 117.262 | 125.646 | 60.782 | 1.00 | 35.92 | E |
| ATOM | 4048 | O5′ | THY | E | 3 | 117.715 | 123.353 | 59.864 | 1.00 | 36.83 | E |
| ATOM | 4049 | N1 | THY | E | 3 | 116.477 | 118.659 | 58.098 | 1.00 | 40.74 | E |
| ATOM | 4050 | C6 | THY | E | 3 | 115.265 | 119.304 | 58.092 | 1.00 | 39.04 | E |
| ATOM | 4051 | C2 | THY | E | 3 | 116.556 | 117.279 | 58.202 | 1.00 | 40.40 | E |
| ATOM | 4052 | O2 | THY | E | 3 | 117.633 | 116.676 | 58.213 | 1.00 | 41.55 | E |
| ATOM | 4053 | N3 | THY | E | 3 | 115.343 | 116.637 | 58.277 | 1.00 | 37.75 | E |
| ATOM | 4054 | C4 | THY | E | 3 | 114.098 | 117.223 | 58.239 | 1.00 | 38.86 | E |
| ATOM | 4055 | O4 | THY | E | 3 | 113.073 | 116.526 | 58.295 | 1.00 | 39.60 | E |
| ATOM | 4056 | C5 | THY | E | 3 | 114.094 | 118.667 | 58.136 | 1.00 | 38.93 | E |
| ATOM | 4057 | C5A | THY | E | 3 | 112.778 | 119.381 | 58.084 | 1.00 | 37.77 | E |
| ATOM | 4058 | C2′ | THY | E | 3 | 117.721 | 120.631 | 57.079 | 1.00 | 40.67 | E |
| ATOM | 4059 | C5′ | THY | E | 3 | 117.470 | 121.961 | 59.952 | 1.00 | 39.89 | E |
| ATOM | 4060 | C4′ | THY | E | 3 | 118.554 | 121.198 | 59.244 | 1.00 | 41.27 | E |
| ATOM | 4061 | O4′ | THY | E | 3 | 118.124 | 119.816 | 59.234 | 1.00 | 40.05 | E |
| ATOM | 4062 | C1′ | THY | E | 3 | 117.753 | 119.396 | 57.954 | 1.00 | 39.78 | E |
| ATOM | 4063 | C3′ | THY | E | 3 | 118.690 | 121.572 | 57.776 | 1.00 | 43.08 | E |
| ATOM | 4064 | O3′ | THY | E | 3 | 120.060 | 121.547 | 57.320 | 1.00 | 46.62 | E |
| ATOM | 4065 | P | GUA | E | 4 | 120.379 | 121.875 | 55.778 | 1.00 | 50.54 | E |
| ATOM | 4066 | O1P | GUA | E | 4 | 119.136 | 122.179 | 54.971 | 1.00 | 46.71 | E |
| ATOM | 4067 | O2P | GUA | E | 4 | 121.506 | 122.796 | 55.781 | 1.00 | 49.34 | E |
| ATOM | 4068 | O5′ | GUA | E | 4 | 120.865 | 120.454 | 55.282 | 1.00 | 49.82 | E |
| ATOM | 4069 | N9 | GUA | E | 4 | 118.682 | 117.585 | 54.409 | 1.00 | 46.18 | E |
| ATOM | 4070 | C4 | GUA | E | 4 | 117.716 | 116.583 | 54.557 | 1.00 | 43.57 | E |
| ATOM | 4071 | N3 | GUA | E | 4 | 117.946 | 115.255 | 54.637 | 1.00 | 41.80 | E |
| ATOM | 4072 | C2 | GUA | E | 4 | 116.812 | 114.563 | 54.705 | 1.00 | 41.88 | E |
| ATOM | 4073 | N2 | GUA | E | 4 | 116.825 | 113.223 | 54.747 | 1.00 | 41.24 | E |
| ATOM | 4074 | N1 | GUA | E | 4 | 115.565 | 115.127 | 54.728 | 1.00 | 41.44 | E |
| ATOM | 4075 | C6 | GUA | E | 4 | 115.310 | 116.485 | 54.664 | 1.00 | 42.52 | E |
| ATOM | 4076 | O6 | GUA | E | 4 | 114.148 | 116.880 | 54.697 | 1.00 | 42.40 | E |
| ATOM | 4077 | C5 | GUA | E | 4 | 116.503 | 117.240 | 54.562 | 1.00 | 42.60 | E |
| ATOM | 4078 | N7 | GUA | E | 4 | 116.683 | 118.606 | 54.439 | 1.00 | 44.80 | E |
| ATOM | 4079 | C8 | GUA | E | 4 | 117.989 | 118.769 | 54.363 | 1.00 | 45.95 | E |
| ATOM | 4080 | C2′ | GUA | E | 4 | 120.885 | 118.301 | 53.340 | 1.00 | 46.23 | E |
| ATOM | 4081 | C5′ | GUA | E | 4 | 121.731 | 119.718 | 56.086 | 1.00 | 47.51 | E |
| ATOM | 4082 | C4′ | GUA | E | 4 | 121.941 | 118.351 | 55.489 | 1.00 | 48.35 | E |
| ATOM | 4083 | O4′ | GUA | E | 4 | 120.701 | 117.628 | 55.595 | 1.00 | 47.78 | E |
| ATOM | 4084 | C1′ | GUA | E | 4 | 120.135 | 117.408 | 54.304 | 1.00 | 47.18 | E |
| ATOM | 4085 | C3′ | GUA | E | 4 | 122.251 | 118.384 | 53.992 | 1.00 | 47.84 | E |
| ATOM | 4086 | O3′ | GUA | E | 4 | 123.105 | 117.313 | 53.657 | 1.00 | 48.82 | E |
| ATOM | 4087 | P | CYT | E | 5 | 123.755 | 117.239 | 52.205 | 1.00 | 51.98 | E |
| ATOM | 4088 | O1P | CYT | E | 5 | 123.487 | 118.481 | 51.434 | 1.00 | 51.40 | E |
| ATOM | 4089 | O2P | CYT | E | 5 | 125.151 | 116.827 | 52.503 | 1.00 | 52.25 | E |
| ATOM | 4090 | O5′ | CYT | E | 5 | 122.959 | 116.037 | 51.530 | 1.00 | 49.54 | E |
| ATOM | 4091 | N1 | CYT | E | 5 | 118.672 | 115.266 | 50.773 | 1.00 | 44.13 | E |
| ATOM | 4092 | C6 | CYT | E | 5 | 118.685 | 116.621 | 50.619 | 1.00 | 42.43 | E |
| ATOM | 4093 | C2 | CYT | E | 5 | 117.480 | 114.614 | 51.156 | 1.00 | 42.08 | E |
| ATOM | 4094 | O2 | CYT | E | 5 | 117.488 | 113.398 | 51.290 | 1.00 | 40.99 | E |
| ATOM | 4095 | N3 | CYT | E | 5 | 116.356 | 115.337 | 51.362 | 1.00 | 40.49 | E |
| ATOM | 4096 | C4 | CYT | E | 5 | 116.383 | 116.650 | 51.205 | 1.00 | 40.45 | E |
| ATOM | 4097 | N4 | CYT | E | 5 | 115.266 | 117.307 | 51.402 | 1.00 | 40.76 | E |
| ATOM | 4098 | C5 | CYT | E | 5 | 117.571 | 117.344 | 50.825 | 1.00 | 41.12 | E |
| ATOM | 4099 | C2′ | CYT | E | 5 | 121.016 | 114.881 | 49.621 | 1.00 | 47.78 | E |
| ATOM | 4100 | C5′ | CYT | E | 5 | 122.778 | 114.872 | 52.284 | 1.00 | 48.31 | E |
| ATOM | 4101 | C4′ | CYT | E | 5 | 121.916 | 113.862 | 51.570 | 1.00 | 48.74 | E |
| ATOM | 4102 | O4′ | CYT | E | 5 | 120.499 | 114.113 | 51.788 | 1.00 | 48.97 | E |
| ATOM | 4103 | C1′ | CYT | E | 5 | 119.889 | 114.442 | 50.544 | 1.00 | 47.01 | E |
| ATOM | 4104 | C3′ | CYT | E | 5 | 122.098 | 113.908 | 50.058 | 1.00 | 48.84 | E |
| ATOM | 4105 | O3′ | CYT | E | 5 | 121.854 | 112.596 | 49.532 | 1.00 | 51.33 | E |
| ATOM | 4106 | P | GUA | E | 6 | 122.422 | 112.200 | 48.086 | 1.00 | 52.42 | E |
| ATOM | 4107 | O1P | GUA | E | 6 | 121.792 | 113.063 | 47.081 | 1.00 | 54.19 | E |
| ATOM | 4108 | O2P | GUA | E | 6 | 123.896 | 112.170 | 48.165 | 1.00 | 53.92 | E |
| ATOM | 4109 | O5′ | GUA | E | 6 | 121.834 | 110.748 | 47.832 | 1.00 | 51.48 | E |
| ATOM | 4110 | N9 | GUA | E | 6 | 117.810 | 111.270 | 47.814 | 1.00 | 51.17 | E |
| ATOM | 4111 | C4 | GUA | E | 6 | 116.477 | 111.555 | 47.984 | 1.00 | 48.87 | E |
| ATOM | 4112 | N3 | GUA | E | 6 | 115.486 | 110.666 | 48.148 | 1.00 | 49.21 | E |
| ATOM | 4113 | C2 | GUA | E | 6 | 114.310 | 111.263 | 48.300 | 1.00 | 49.79 | E |
| ATOM | 4114 | N2 | GUA | E | 6 | 113.181 | 110.543 | 48.465 | 1.00 | 49.22 | E |
| ATOM | 4115 | N1 | GUA | E | 6 | 114.142 | 112.614 | 48.298 | 1.00 | 49.07 | E |
| ATOM | 4116 | C6 | GUA | E | 6 | 115.157 | 113.528 | 48.125 | 1.00 | 49.70 | E |
| ATOM | 4117 | O6 | GUA | E | 6 | 114.911 | 114.715 | 48.133 | 1.00 | 51.34 | E |
| ATOM | 4118 | C5 | GUA | E | 6 | 116.394 | 112.915 | 47.958 | 1.00 | 49.64 | E |
| ATOM | 4119 | N7 | GUA | E | 6 | 117.645 | 113.484 | 47.797 | 1.00 | 50.86 | E |
| ATOM | 4120 | C8 | GUA | E | 6 | 118.455 | 112.473 | 47.724 | 1.00 | 51.45 | E |
| ATOM | 4121 | C2′ | GUA | E | 6 | 119.416 | 109.796 | 46.564 | 1.00 | 52.27 | E |
| ATOM | 4122 | C5′ | GUA | E | 6 | 121.570 | 109.879 | 48.900 | 1.00 | 51.53 | E |
| ATOM | 4123 | C4′ | GUA | E | 6 | 120.381 | 108.996 | 48.582 | 1.00 | 52.78 | E |
| ATOM | 4124 | O4′ | GUA | E | 6 | 119.135 | 109.656 | 48.909 | 1.00 | 52.56 | E |
| ATOM | 4125 | C1′ | GUA | E | 6 | 118.427 | 109.949 | 47.705 | 1.00 | 52.26 | E |
| ATOM | 4126 | C3′ | GUA | E | 6 | 120.264 | 108.660 | 47.093 | 1.00 | 53.40 | E |
| ATOM | 4127 | O3′ | GUA | E | 6 | 119.496 | 107.459 | 46.958 | 1.00 | 54.45 | E |
| ATOM | 4128 | P | GUA | E | 7 | 119.382 | 106.711 | 45.526 | 1.00 | 55.30 | E |
| ATOM | 4129 | O1P | GUA | E | 7 | 119.320 | 107.683 | 44.427 | 1.00 | 54.94 | E |
| ATOM | 4130 | O2P | GUA | E | 7 | 120.433 | 105.653 | 45.503 | 1.00 | 57.67 | E |
| ATOM | 4131 | O5′ | GUA | E | 7 | 117.943 | 106.058 | 45.643 | 1.00 | 52.72 | E |
| ATOM | 4132 | N9 | GUA | E | 7 | 114.830 | 109.617 | 44.782 | 1.00 | 46.05 | E |
| ATOM | 4133 | C4 | GUA | E | 7 | 113.922 | 110.630 | 44.844 | 1.00 | 44.11 | E |
| ATOM | 4134 | N3 | GUA | E | 7 | 112.583 | 110.498 | 44.924 | 1.00 | 42.20 | E |
| ATOM | 4135 | C2 | GUA | E | 7 | 111.978 | 111.678 | 44.913 | 1.00 | 41.66 | E |
| ATOM | 4136 | N2 | GUA | E | 7 | 110.667 | 111.759 | 44.948 | 1.00 | 40.58 | E |
| ATOM | 4137 | N1 | GUA | E | 7 | 112.627 | 112.881 | 44.856 | 1.00 | 42.07 | E |
| ATOM | 4138 | C6 | GUA | E | 7 | 113.994 | 113.042 | 44.788 | 1.00 | 43.56 | E |
| ATOM | 4139 | O6 | GUA | E | 7 | 114.470 | 114.166 | 44.749 | 1.00 | 45.17 | E |
| ATOM | 4140 | C5 | GUA | E | 7 | 114.667 | 111.791 | 44.774 | 1.00 | 44.65 | E |
| ATOM | 4141 | N7 | GUA | E | 7 | 116.023 | 111.504 | 44.708 | 1.00 | 45.17 | E |
| ATOM | 4142 | C8 | GUA | E | 7 | 116.067 | 110.203 | 44.736 | 1.00 | 45.80 | E |
| ATOM | 4143 | C2′ | GUA | E | 7 | 115.444 | 107.468 | 43.703 | 1.00 | 51.37 | E |
| ATOM | 4144 | C5′ | GUA | E | 7 | 116.967 | 106.765 | 46.338 | 1.00 | 51.30 | E |
| ATOM | 4145 | C4′ | GUA | E | 7 | 115.603 | 106.421 | 45.819 | 1.00 | 52.06 | E |
| ATOM | 4146 | O4′ | GUA | E | 7 | 114.811 | 107.620 | 45.966 | 1.00 | 51.99 | E |
| ATOM | 4147 | C1′ | GUA | E | 7 | 114.554 | 108.195 | 44.702 | 1.00 | 49.77 | E |
| ATOM | 4148 | C3′ | GUA | E | 7 | 115.562 | 106.094 | 44.331 | 1.00 | 53.43 | E |
| ATOM | 4149 | O3′ | GUA | E | 7 | 114.377 | 105.272 | 44.086 | 1.00 | 55.78 | E |
| ATOM | 4150 | P | THY | E | 8 | 114.029 | 104.729 | 42.581 | 1.00 | 56.78 | E |
| ATOM | 4151 | O1P | THY | E | 8 | 115.157 | 105.041 | 41.669 | 1.00 | 54.64 | E |
| ATOM | 4152 | O2P | THY | E | 8 | 113.532 | 103.354 | 42.714 | 1.00 | 58.16 | E |
| ATOM | 4153 | O5′ | THY | E | 8 | 112.787 | 105.606 | 42.150 | 1.00 | 56.47 | E |
| ATOM | 4154 | N1 | THY | E | 8 | 112.613 | 110.808 | 41.289 | 1.00 | 48.69 | E |
| ATOM | 4155 | C6 | THY | E | 8 | 113.923 | 110.419 | 41.249 | 1.00 | 46.91 | E |
| ATOM | 4156 | C2 | THY | E | 8 | 112.271 | 112.135 | 41.440 | 1.00 | 48.21 | E |
| ATOM | 4157 | O2 | THY | E | 8 | 111.103 | 112.536 | 41.486 | 1.00 | 49.50 | E |
| ATOM | 4158 | N3 | THY | E | 8 | 113.336 | 112.984 | 41.543 | 1.00 | 45.47 | E |
| ATOM | 4159 | C4 | THY | E | 8 | 114.657 | 112.668 | 41.502 | 1.00 | 45.05 | E |
| ATOM | 4160 | O4 | THY | E | 8 | 115.502 | 113.548 | 41.605 | 1.00 | 44.75 | E |
| ATOM | 4161 | C5 | THY | E | 8 | 114.946 | 111.277 | 41.337 | 1.00 | 46.23 | E |
| ATOM | 4162 | C5A | THY | E | 8 | 116.373 | 110.853 | 41.261 | 1.00 | 44.33 | E |
| ATOM | 4163 | C2′ | THY | E | 8 | 111.624 | 108.888 | 39.993 | 1.00 | 52.83 | E |
| ATOM | 4164 | C5′ | THY | E | 8 | 112.877 | 107.019 | 42.220 | 1.00 | 55.81 | E |
| ATOM | 4165 | C4′ | THY | E | 8 | 111.517 | 107.625 | 42.014 | 1.00 | 55.14 | E |
| ATOM | 4166 | O4′ | THY | E | 8 | 111.615 | 109.029 | 42.360 | 1.00 | 53.89 | E |
| ATOM | 4167 | C1′ | THY | E | 8 | 111.539 | 109.830 | 41.189 | 1.00 | 50.84 | E |
| ATOM | 4168 | C3′ | THY | E | 8 | 111.071 | 107.586 | 40.548 | 1.00 | 54.85 | E |
| ATOM | 4169 | O3′ | THY | E | 8 | 109.651 | 107.492 | 40.502 | 1.00 | 57.02 | E |
| ATOM | 4170 | P | THY | E | 9 | 108.896 | 107.207 | 39.105 | 1.00 | 59.12 | E |
| ATOM | 4171 | O1P | THY | E | 9 | 109.941 | 107.007 | 38.078 | 1.00 | 54.99 | E |
| ATOM | 4172 | O2P | THY | E | 9 | 107.802 | 106.177 | 39.356 | 1.00 | 59.06 | E |
| ATOM | 4173 | O5′ | THY | E | 9 | 108.111 | 108.557 | 38.822 | 1.00 | 59.66 | E |
| ATOM | 4174 | N1 | THY | E | 9 | 110.364 | 112.631 | 37.780 | 1.00 | 52.32 | E |
| ATOM | 4175 | C6 | THY | E | 9 | 111.258 | 111.602 | 37.611 | 1.00 | 49.64 | E |
| ATOM | 4176 | C2 | THY | E | 9 | 110.788 | 113.926 | 37.982 | 1.00 | 51.42 | E |
| ATOM | 4177 | O2 | THY | E | 9 | 110.010 | 114.865 | 38.120 | 1.00 | 50.86 | E |
| ATOM | 4178 | N3 | THY | E | 9 | 112.154 | 114.084 | 38.017 | 1.00 | 50.87 | E |
| ATOM | 4179 | C4 | THY | E | 9 | 113.115 | 113.091 | 37.876 | 1.00 | 49.21 | E |
| ATOM | 4180 | O4 | THY | E | 9 | 114.330 | 113.365 | 37.977 | 1.00 | 46.45 | E |
| ATOM | 4181 | C5 | THY | E | 9 | 112.586 | 111.769 | 37.640 | 1.00 | 48.94 | E |
| ATOM | 4182 | C5A | THY | E | 9 | 113.551 | 110.652 | 37.436 | 1.00 | 47.63 | E |
| ATOM | 4183 | C2′ | THY | E | 9 | 108.435 | 111.293 | 36.829 | 1.00 | 56.40 | E |
| ATOM | 4184 | C5′ | THY | E | 9 | 108.461 | 109.740 | 39.527 | 1.00 | 59.72 | E |
| ATOM | 4185 | C4′ | THY | E | 9 | 107.641 | 110.895 | 39.026 | 1.00 | 58.28 | E |
| ATOM | 4186 | O4′ | THY | E | 9 | 108.515 | 112.046 | 39.074 | 1.00 | 56.24 | E |
| ATOM | 4187 | C1′ | THY | E | 9 | 108.903 | 112.405 | 37.763 | 1.00 | 55.17 | E |
| ATOM | 4188 | C3′ | THY | E | 9 | 107.244 | 110.727 | 37.562 | 1.00 | 58.18 | E |
| ATOM | 4189 | O3′ | THY | E | 9 | 106.044 | 111.464 | 37.305 | 1.00 | 61.06 | E |
| ATOM | 4190 | P | GUA | E | 10 | 105.322 | 111.412 | 35.833 | 1.00 | 63.68 | E |
| ATOM | 4191 | O1P | GUA | E | 10 | 106.043 | 110.543 | 34.880 | 1.00 | 60.35 | E |
| ATOM | 4192 | O2P | GUA | E | 10 | 103.859 | 111.135 | 36.141 | 1.00 | 62.38 | E |
| ATOM | 4193 | O5′ | GUA | E | 10 | 105.462 | 112.927 | 35.334 | 1.00 | 61.83 | E |
| ATOM | 4194 | N9 | GUA | E | 10 | 108.312 | 115.202 | 34.491 | 1.00 | 50.74 | E |
| ATOM | 4195 | C4 | GUA | E | 10 | 109.501 | 115.891 | 34.652 | 1.00 | 48.21 | E |
| ATOM | 4196 | N3 | GUA | E | 10 | 109.660 | 117.233 | 34.686 | 1.00 | 48.49 | E |
| ATOM | 4197 | C2 | GUA | E | 10 | 110.948 | 117.591 | 34.857 | 1.00 | 46.90 | E |
| ATOM | 4198 | N2 | GUA | E | 10 | 111.298 | 118.899 | 34.938 | 1.00 | 45.76 | E |
| ATOM | 4199 | N1 | GUA | E | 10 | 111.971 | 116.703 | 34.966 | 1.00 | 44.95 | E |
| ATOM | 4200 | C6 | GUA | E | 10 | 111.824 | 115.323 | 34.942 | 1.00 | 47.20 | E |
| ATOM | 4201 | O6 | GUA | E | 10 | 112.810 | 114.606 | 35.073 | 1.00 | 46.31 | E |
| ATOM | 4202 | C5 | GUA | E | 10 | 110.465 | 114.927 | 34.767 | 1.00 | 47.40 | E |
| ATOM | 4203 | N7 | GUA | E | 10 | 109.902 | 113.656 | 34.709 | 1.00 | 50.27 | E |
| ATOM | 4204 | C8 | GUA | E | 10 | 108.618 | 113.874 | 34.564 | 1.00 | 50.64 | E |
| ATOM | 4205 | C2′ | GUA | E | 10 | 106.125 | 114.848 | 33.376 | 1.00 | 56.61 | E |
| ATOM | 4206 | C5′ | GUA | E | 10 | 104.964 | 113.989 | 36.152 | 1.00 | 60.56 | E |
| ATOM | 4207 | C4′ | GUA | E | 10 | 105.024 | 115.298 | 35.407 | 1.00 | 58.95 | E |
| ATOM | 4208 | O4′ | GUA | E | 10 | 106.364 | 115.829 | 35.489 | 1.00 | 58.06 | E |
| ATOM | 4209 | C1′ | GUA | E | 10 | 106.997 | 115.745 | 34.245 | 1.00 | 53.76 | E |
| ATOM | 4210 | C3′ | GUA | E | 10 | 104.745 | 115.141 | 33.916 | 1.00 | 58.06 | E |
| ATOM | 4211 | O3′ | GUA | E | 10 | 104.364 | 116.389 | 33.272 | 1.00 | 58.47 | E |
| ATOM | 4212 | O5′ | CYT | F | 1 | 119.350 | 120.486 | 36.060 | 1.00 | 56.04 | F |
| ATOM | 4213 | N1 | CYT | F | 1 | 116.020 | 119.550 | 36.039 | 1.00 | 43.78 | F |
| ATOM | 4214 | C6 | CYT | F | 1 | 117.000 | 118.649 | 36.338 | 1.00 | 41.77 | F |
| ATOM | 4215 | C2 | CYT | F | 1 | 114.748 | 119.103 | 35.628 | 1.00 | 44.46 | F |
| ATOM | 4216 | O2 | CYT | F | 1 | 113.857 | 119.930 | 35.358 | 1.00 | 45.95 | F |
| ATOM | 4217 | N3 | CYT | F | 1 | 114.517 | 117.777 | 35.516 | 1.00 | 44.41 | F |
| ATOM | 4218 | C4 | CYT | F | 1 | 115.487 | 116.910 | 35.789 | 1.00 | 43.40 | F |
| ATOM | 4219 | N4 | CYT | F | 1 | 115.206 | 115.630 | 35.631 | 1.00 | 44.63 | F |
| ATOM | 4220 | C5 | CYT | F | 1 | 116.781 | 117.327 | 36.228 | 1.00 | 41.50 | F |
| ATOM | 4221 | C2′ | CYT | F | 1 | 117.109 | 121.486 | 37.273 | 1.00 | 46.79 | F |
| ATOM | 4222 | C5′ | CYT | F | 1 | 119.415 | 121.528 | 35.089 | 1.00 | 52.32 | F |
| ATOM | 4223 | C4′ | CYT | F | 1 | 118.095 | 122.249 | 35.238 | 1.00 | 51.35 | F |
| ATOM | 4224 | O4′ | CYT | F | 1 | 116.987 | 121.351 | 34.919 | 1.00 | 50.23 | F |
| ATOM | 4225 | C1′ | CYT | F | 1 | 116.272 | 120.988 | 36.105 | 1.00 | 46.19 | F |
| ATOM | 4226 | C3′ | CYT | F | 1 | 117.843 | 122.687 | 36.681 | 1.00 | 48.80 | F |
| ATOM | 4227 | O3′ | CYT | F | 1 | 117.070 | 123.910 | 36.619 | 1.00 | 49.85 | F |
| ATOM | 4228 | P | ADE | F | 2 | 116.772 | 124.786 | 37.963 | 1.00 | 48.88 | F |
| ATOM | 4229 | O1P | ADE | F | 2 | 117.852 | 124.618 | 38.936 | 1.00 | 46.61 | F |
| ATOM | 4230 | O2P | ADE | F | 2 | 116.506 | 126.146 | 37.362 | 1.00 | 49.56 | F |
| ATOM | 4231 | O5′ | ADE | F | 2 | 115.413 | 124.156 | 38.539 | 1.00 | 45.15 | F |
| ATOM | 4232 | N9 | ADE | F | 2 | 114.024 | 120.470 | 39.699 | 1.00 | 44.04 | F |
| ATOM | 4233 | C4 | ADE | F | 2 | 113.530 | 119.260 | 39.297 | 1.00 | 42.59 | F |
| ATOM | 4234 | N3 | ADE | F | 2 | 112.274 | 118.986 | 38.916 | 1.00 | 43.47 | F |
| ATOM | 4235 | C2 | ADE | F | 2 | 112.147 | 117.701 | 38.569 | 1.00 | 42.92 | F |
| ATOM | 4236 | N1 | ADE | F | 2 | 113.065 | 116.736 | 38.549 | 1.00 | 42.36 | F |
| ATOM | 4237 | C6 | ADE | F | 2 | 114.330 | 117.044 | 38.927 | 1.00 | 42.82 | F |
| ATOM | 4238 | N6 | ADE | F | 2 | 115.254 | 116.088 | 38.880 | 1.00 | 40.40 | F |
| ATOM | 4239 | C5 | ADE | F | 2 | 114.591 | 118.382 | 39.345 | 1.00 | 43.31 | F |
| ATOM | 4240 | N7 | ADE | F | 2 | 115.737 | 119.015 | 39.813 | 1.00 | 41.93 | F |
| ATOM | 4241 | C8 | ADE | F | 2 | 115.348 | 120.245 | 40.011 | 1.00 | 43.96 | F |
| ATOM | 4242 | C2′ | ADE | F | 2 | 113.704 | 122.876 | 40.588 | 1.00 | 43.64 | F |
| ATOM | 4243 | C5′ | ADE | F | 2 | 114.230 | 124.310 | 37.778 | 1.00 | 44.39 | F |
| ATOM | 4244 | C4′ | ADE | F | 2 | 113.038 | 123.670 | 38.443 | 1.00 | 45.04 | F |
| ATOM | 4245 | O4′ | ADE | F | 2 | 113.101 | 122.217 | 38.402 | 1.00 | 45.64 | F |
| ATOM | 4246 | C1′ | ADE | F | 2 | 113.240 | 121.714 | 39.733 | 1.00 | 44.50 | F |
| ATOM | 4247 | C3′ | ADE | F | 2 | 112.959 | 124.014 | 39.924 | 1.00 | 43.54 | F |
| ATOM | 4248 | O3′ | ADE | F | 2 | 111.591 | 124.029 | 40.322 | 1.00 | 42.75 | F |
| ATOM | 4249 | P | ADE | F | 3 | 111.125 | 125.082 | 41.422 | 1.00 | 43.07 | F |
| ATOM | 4250 | O1P | ADE | F | 3 | 112.293 | 125.170 | 42.346 | 1.00 | 41.00 | F |
| ATOM | 4251 | O2P | ADE | F | 3 | 110.616 | 126.303 | 40.758 | 1.00 | 44.81 | F |
| ATOM | 4252 | O5′ | ADE | F | 3 | 109.934 | 124.322 | 42.090 | 1.00 | 39.17 | F |
| ATOM | 4253 | N9 | ADE | F | 3 | 110.931 | 119.303 | 41.892 | 1.00 | 37.38 | F |
| ATOM | 4254 | C4 | ADE | F | 3 | 111.325 | 118.003 | 41.756 | 1.00 | 37.30 | F |
| ATOM | 4255 | N3 | ADE | F | 3 | 110.591 | 116.993 | 41.248 | 1.00 | 37.47 | F |
| ATOM | 4256 | C2 | ADE | F | 3 | 111.281 | 115.879 | 41.278 | 1.00 | 36.21 | F |
| ATOM | 4257 | N1 | ADE | F | 3 | 112.535 | 115.671 | 41.724 | 1.00 | 37.94 | F |
| ATOM | 4258 | C6 | ADE | F | 3 | 113.254 | 116.699 | 42.222 | 1.00 | 34.83 | F |
| ATOM | 4259 | N6 | ADE | F | 3 | 114.498 | 116.451 | 42.636 | 1.00 | 29.59 | F |
| ATOM | 4260 | C5 | ADE | F | 3 | 112.624 | 117.946 | 42.252 | 1.00 | 36.24 | F |
| ATOM | 4261 | N7 | ADE | F | 3 | 113.045 | 119.199 | 42.658 | 1.00 | 36.50 | F |
| ATOM | 4262 | C8 | ADE | F | 3 | 112.016 | 119.969 | 42.408 | 1.00 | 37.37 | F |
| ATOM | 4263 | C2′ | ADE | F | 3 | 109.103 | 120.005 | 43.034 | 1.00 | 38.45 | F |
| ATOM | 4264 | C5′ | ADE | F | 3 | 110.147 | 123.007 | 42.505 | 1.00 | 38.89 | F |
| ATOM | 4265 | C4′ | ADE | F | 3 | 109.072 | 122.151 | 41.917 | 1.00 | 38.50 | F |
| ATOM | 4266 | O4′ | ADE | F | 3 | 109.742 | 121.135 | 41.091 | 1.00 | 36.78 | F |
| ATOM | 4267 | C1′ | ADE | F | 3 | 109.603 | 119.829 | 41.644 | 1.00 | 35.83 | F |
| ATOM | 4268 | C3′ | ADE | F | 3 | 108.451 | 121.349 | 43.032 | 1.00 | 37.95 | F |
| ATOM | 4269 | O3′ | ADE | F | 3 | 107.152 | 121.532 | 43.498 | 1.00 | 40.03 | F |
| ATOM | 4270 | P | CYT | F | 4 | 106.880 | 121.376 | 45.084 | 1.00 | 39.01 | F |
| ATOM | 4271 | O1P | CYT | F | 4 | 108.140 | 121.756 | 45.771 | 1.00 | 36.65 | F |
| ATOM | 4272 | O2P | CYT | F | 4 | 105.615 | 122.096 | 45.377 | 1.00 | 39.29 | F |
| ATOM | 4273 | O5′ | CYT | F | 4 | 106.585 | 119.819 | 45.250 | 1.00 | 37.97 | F |
| ATOM | 4274 | N1 | CYT | F | 4 | 109.010 | 116.364 | 44.877 | 1.00 | 40.52 | F |
| ATOM | 4275 | C6 | CYT | F | 4 | 109.621 | 117.556 | 45.140 | 1.00 | 39.78 | F |
| ATOM | 4276 | C2 | CYT | F | 4 | 109.762 | 115.242 | 44.618 | 1.00 | 40.24 | F |
| ATOM | 4277 | O2 | CYT | F | 4 | 109.178 | 114.193 | 44.281 | 1.00 | 41.34 | F |
| ATOM | 4278 | N3 | CYT | F | 4 | 111.103 | 115.314 | 44.742 | 1.00 | 40.07 | F |
| ATOM | 4279 | C4 | CYT | F | 4 | 111.683 | 116.455 | 45.099 | 1.00 | 39.41 | F |
| ATOM | 4280 | N4 | CYT | F | 4 | 113.003 | 116.456 | 45.304 | 1.00 | 37.56 | F |
| ATOM | 4281 | C5 | CYT | F | 4 | 110.939 | 117.638 | 45.279 | 1.00 | 39.55 | F |
| ATOM | 4282 | C2′ | CYT | F | 4 | 107.007 | 116.868 | 46.235 | 1.00 | 42.10 | F |
| ATOM | 4283 | C5′ | CYT | F | 4 | 106.045 | 119.137 | 44.132 | 1.00 | 40.14 | F |
| ATOM | 4284 | C4′ | CYT | F | 4 | 105.793 | 117.664 | 44.373 | 1.00 | 41.36 | F |
| ATOM | 4285 | O4′ | CYT | F | 4 | 106.951 | 116.895 | 43.915 | 1.00 | 39.45 | F |
| ATOM | 4286 | C1′ | CYT | F | 4 | 107.551 | 116.228 | 44.980 | 1.00 | 39.38 | F |
| ATOM | 4287 | C3′ | CYT | F | 4 | 105.624 | 117.299 | 45.835 | 1.00 | 41.80 | F |
| ATOM | 4288 | O3′ | CYT | F | 4 | 104.680 | 116.295 | 46.085 | 1.00 | 46.11 | F |
| ATOM | 4289 | P | CYT | F | 5 | 104.296 | 115.931 | 47.620 | 1.00 | 52.17 | F |
| ATOM | 4290 | O1P | CYT | F | 5 | 104.861 | 117.012 | 48.499 | 1.00 | 50.87 | F |
| ATOM | 4291 | O2P | CYT | F | 5 | 102.850 | 115.569 | 47.692 | 1.00 | 49.75 | F |
| ATOM | 4292 | O5′ | CYT | F | 5 | 105.125 | 114.603 | 47.927 | 1.00 | 50.70 | F |
| ATOM | 4293 | N1 | CYT | F | 5 | 109.082 | 113.416 | 48.643 | 1.00 | 42.73 | F |
| ATOM | 4294 | C6 | CYT | F | 5 | 108.916 | 114.775 | 48.760 | 1.00 | 41.17 | F |
| ATOM | 4295 | C2 | CYT | F | 5 | 110.364 | 112.871 | 48.499 | 1.00 | 40.46 | F |
| ATOM | 4296 | O2 | CYT | F | 5 | 110.486 | 111.651 | 48.406 | 1.00 | 38.48 | F |
| ATOM | 4297 | N3 | CYT | F | 5 | 111.442 | 113.716 | 48.471 | 1.00 | 38.96 | F |
| ATOM | 4298 | C4 | CYT | F | 5 | 111.259 | 115.043 | 48.592 | 1.00 | 36.76 | F |
| ATOM | 4299 | N4 | CYT | F | 5 | 112.328 | 115.830 | 48.569 | 1.00 | 33.81 | F |
| ATOM | 4300 | C5 | CYT | F | 5 | 109.967 | 115.614 | 48.740 | 1.00 | 37.82 | F |
| ATOM | 4301 | C2′ | CYT | F | 5 | 106.799 | 112.772 | 49.643 | 1.00 | 46.97 | F |
| ATOM | 4302 | C5′ | CYT | F | 5 | 105.189 | 113.604 | 46.950 | 1.00 | 48.60 | F |
| ATOM | 4303 | C4′ | CYT | F | 5 | 105.878 | 112.368 | 47.464 | 1.00 | 48.82 | F |
| ATOM | 4304 | O4′ | CYT | F | 5 | 107.327 | 112.535 | 47.353 | 1.00 | 48.88 | F |
| ATOM | 4305 | C1′ | CYT | F | 5 | 107.928 | 112.512 | 48.654 | 1.00 | 45.33 | F |
| ATOM | 4306 | C3′ | CYT | F | 5 | 105.618 | 112.089 | 48.954 | 1.00 | 49.22 | F |
| ATOM | 4307 | O3′ | CYT | F | 5 | 105.524 | 110.657 | 49.187 | 1.00 | 52.62 | F |
| ATOM | 4308 | P | GUA | F | 6 | 104.788 | 110.074 | 50.519 | 1.00 | 56.66 | F |
| ATOM | 4309 | O1P | GUA | F | 6 | 104.243 | 111.195 | 51.337 | 1.00 | 56.04 | F |
| ATOM | 4310 | O2P | GUA | F | 6 | 103.915 | 108.930 | 50.167 | 1.00 | 57.78 | F |
| ATOM | 4311 | O5′ | GUA | F | 6 | 106.015 | 109.523 | 51.327 | 1.00 | 55.45 | F |
| ATOM | 4312 | N9 | GUA | F | 6 | 110.542 | 111.901 | 52.171 | 1.00 | 46.06 | F |
| ATOM | 4313 | C4 | GUA | F | 6 | 111.792 | 112.401 | 51.937 | 1.00 | 42.47 | F |
| ATOM | 4314 | N3 | GUA | F | 6 | 112.907 | 111.670 | 51.757 | 1.00 | 41.10 | F |
| ATOM | 4315 | C2 | GUA | F | 6 | 113.964 | 112.438 | 51.563 | 1.00 | 41.59 | F |
| ATOM | 4316 | N2 | GUA | F | 6 | 115.164 | 111.872 | 51.315 | 1.00 | 40.80 | F |
| ATOM | 4317 | N1 | GUA | F | 6 | 113.930 | 113.828 | 51.588 | 1.00 | 40.51 | F |
| ATOM | 4318 | C6 | GUA | F | 6 | 112.795 | 114.588 | 51.807 | 1.00 | 39.90 | F |
| ATOM | 4319 | O6 | GUA | F | 6 | 112.883 | 115.799 | 51.852 | 1.00 | 40.19 | F |
| ATOM | 4320 | C5 | GUA | F | 6 | 111.648 | 113.782 | 51.963 | 1.00 | 41.12 | F |
| ATOM | 4321 | N7 | GUA | F | 6 | 110.319 | 114.138 | 52.153 | 1.00 | 40.58 | F |
| ATOM | 4322 | C8 | GUA | F | 6 | 109.696 | 112.994 | 52.262 | 1.00 | 43.87 | F |
| ATOM | 4323 | C2′ | GUA | F | 6 | 109.230 | 110.222 | 53.532 | 1.00 | 51.73 | F |
| ATOM | 4324 | C5′ | GUA | F | 6 | 107.239 | 110.188 | 51.160 | 1.00 | 55.85 | F |
| ATOM | 4325 | C4′ | GUA | F | 6 | 108.365 | 109.279 | 51.535 | 1.00 | 54.23 | F |
| ATOM | 4326 | O4′ | GUA | F | 6 | 109.563 | 110.024 | 51.204 | 1.00 | 52.98 | F |
| ATOM | 4327 | C1′ | GUA | F | 6 | 110.207 | 110.484 | 52.373 | 1.00 | 49.99 | F |
| ATOM | 4328 | C3′ | GUA | F | 6 | 108.375 | 109.068 | 53.044 | 1.00 | 53.68 | F |
| ATOM | 4329 | O3′ | GUA | F | 6 | 108.785 | 107.735 | 53.388 | 1.00 | 54.85 | F |
| ATOM | 4330 | P | CYT | F | 7 | 108.639 | 107.198 | 54.913 | 1.00 | 56.27 | F |
| ATOM | 4331 | O1P | CYT | F | 7 | 107.523 | 107.829 | 55.661 | 1.00 | 53.65 | F |
| ATOM | 4332 | O2P | CYT | F | 7 | 108.632 | 105.738 | 54.684 | 1.00 | 55.09 | F |
| ATOM | 4333 | O5′ | CYT | F | 7 | 110.053 | 107.612 | 55.535 | 1.00 | 54.90 | F |
| ATOM | 4334 | N1 | CYT | F | 7 | 112.488 | 111.340 | 55.846 | 1.00 | 54.11 | F |
| ATOM | 4335 | C6 | CYT | F | 7 | 111.234 | 111.872 | 55.834 | 1.00 | 53.28 | F |
| ATOM | 4336 | C2 | CYT | F | 7 | 113.592 | 112.131 | 55.536 | 1.00 | 54.09 | F |
| ATOM | 4337 | O2 | CYT | F | 7 | 114.719 | 111.611 | 55.541 | 1.00 | 54.58 | F |
| ATOM | 4338 | N3 | CYT | F | 7 | 113.418 | 113.436 | 55.253 | 1.00 | 53.81 | F |
| ATOM | 4339 | C4 | CYT | F | 7 | 112.199 | 113.958 | 55.277 | 1.00 | 53.62 | F |
| ATOM | 4340 | N4 | CYT | F | 7 | 112.086 | 115.260 | 55.013 | 1.00 | 53.83 | F |
| ATOM | 4341 | C5 | CYT | F | 7 | 111.044 | 113.170 | 55.570 | 1.00 | 52.55 | F |
| ATOM | 4342 | C2′ | CYT | F | 7 | 111.856 | 109.219 | 57.245 | 1.00 | 56.35 | F |
| ATOM | 4343 | C5′ | CYT | F | 7 | 111.231 | 107.301 | 54.782 | 1.00 | 55.05 | F |
| ATOM | 4344 | C4′ | CYT | F | 7 | 112.497 | 107.767 | 55.467 | 1.00 | 56.73 | F |
| ATOM | 4345 | O4′ | CYT | F | 7 | 112.844 | 109.128 | 55.082 | 1.00 | 56.30 | F |
| ATOM | 4346 | C1′ | CYT | F | 7 | 112.736 | 109.947 | 56.231 | 1.00 | 55.41 | F |
| ATOM | 4347 | C3′ | CYT | F | 7 | 112.344 | 107.799 | 56.994 | 1.00 | 57.75 | F |
| ATOM | 4348 | O3′ | CYT | F | 7 | 113.652 | 107.599 | 57.580 | 1.00 | 58.83 | F |
| ATOM | 4349 | P | ADE | F | 8 | 113.900 | 106.360 | 58.572 | 1.00 | 59.21 | F |
| ATOM | 4350 | O1P | ADE | F | 8 | 113.337 | 106.653 | 59.939 | 1.00 | 57.75 | F |
| ATOM | 4351 | O2P | ADE | F | 8 | 113.414 | 105.185 | 57.786 | 1.00 | 57.99 | F |
| ATOM | 4352 | O5′ | ADE | F | 8 | 115.472 | 106.337 | 58.710 | 1.00 | 56.25 | F |
| ATOM | 4353 | N9 | ADE | F | 8 | 116.111 | 110.019 | 59.004 | 1.00 | 50.03 | F |
| ATOM | 4354 | C4 | ADE | F | 8 | 116.096 | 111.374 | 58.741 | 1.00 | 48.47 | F |
| ATOM | 4355 | N3 | ADE | F | 8 | 117.118 | 112.148 | 58.335 | 1.00 | 47.78 | F |
| ATOM | 4356 | C2 | ADE | F | 8 | 116.723 | 113.393 | 58.155 | 1.00 | 45.82 | F |
| ATOM | 4357 | N1 | ADE | F | 8 | 115.510 | 113.921 | 58.305 | 1.00 | 45.91 | F |
| ATOM | 4358 | C6 | ADE | F | 8 | 114.501 | 113.119 | 58.701 | 1.00 | 46.93 | F |
| ATOM | 4359 | N6 | ADE | F | 8 | 113.283 | 113.629 | 58.820 | 1.00 | 44.82 | F |
| ATOM | 4360 | C5 | ADE | F | 8 | 114.796 | 111.781 | 58.951 | 1.00 | 48.18 | F |
| ATOM | 4361 | N7 | ADE | F | 8 | 114.011 | 110.721 | 59.368 | 1.00 | 49.17 | F |
| ATOM | 4362 | C8 | ADE | F | 8 | 114.844 | 109.704 | 59.393 | 1.00 | 50.24 | F |
| ATOM | 4363 | C2′ | ADE | F | 8 | 117.503 | 108.004 | 59.877 | 1.00 | 54.38 | F |
| ATOM | 4364 | C5′ | ADE | F | 8 | 116.256 | 106.187 | 57.567 | 1.00 | 54.79 | F |
| ATOM | 4365 | C4′ | ADE | F | 8 | 117.477 | 107.062 | 57.676 | 1.00 | 55.23 | F |
| ATOM | 4366 | O4′ | ADE | F | 8 | 117.074 | 108.451 | 57.594 | 1.00 | 55.19 | F |
| ATOM | 4367 | C1′ | ADE | F | 8 | 117.247 | 109.110 | 58.841 | 1.00 | 53.06 | F |
| ATOM | 4368 | C3′ | ADE | F | 8 | 118.194 | 106.940 | 59.020 | 1.00 | 55.52 | F |
| ATOM | 4369 | O3′ | ADE | F | 8 | 119.586 | 107.195 | 58.768 | 1.00 | 56.00 | F |
| ATOM | 4370 | P | ADE | F | 9 | 120.681 | 106.682 | 59.793 | 1.00 | 56.86 | F |
| ATOM | 4371 | O1P | ADE | F | 9 | 119.968 | 105.960 | 60.891 | 1.00 | 56.30 | F |
| ATOM | 4372 | O2P | ADE | F | 9 | 121.729 | 106.022 | 58.998 | 1.00 | 56.33 | F |
| ATOM | 4373 | O5′ | ADE | F | 9 | 121.294 | 108.030 | 60.376 | 1.00 | 55.98 | F |
| ATOM | 4374 | N9 | ADE | F | 9 | 118.510 | 111.739 | 62.025 | 1.00 | 47.93 | F |
| ATOM | 4375 | C4 | ADE | F | 9 | 117.757 | 112.869 | 61.851 | 1.00 | 45.74 | F |
| ATOM | 4376 | N3 | ADE | F | 9 | 118.209 | 114.106 | 61.618 | 1.00 | 44.88 | F |
| ATOM | 4377 | C2 | ADE | F | 9 | 117.216 | 114.972 | 61.577 | 1.00 | 43.09 | F |
| ATOM | 4378 | N1 | ADE | F | 9 | 115.909 | 114.765 | 61.732 | 1.00 | 44.48 | F |
| ATOM | 4379 | C6 | ADE | F | 9 | 115.474 | 113.512 | 61.962 | 1.00 | 45.65 | F |
| ATOM | 4380 | N6 | ADE | F | 9 | 114.153 | 113.318 | 62.134 | 1.00 | 43.58 | F |
| ATOM | 4381 | C5 | ADE | F | 9 | 116.451 | 112.485 | 62.020 | 1.00 | 46.03 | F |
| ATOM | 4382 | N7 | ADE | F | 9 | 116.369 | 111.113 | 62.249 | 1.00 | 48.14 | F |
| ATOM | 4383 | C8 | ADE | F | 9 | 117.619 | 110.719 | 62.224 | 1.00 | 48.22 | F |
| ATOM | 4384 | C2′ | ADE | F | 9 | 120.585 | 110.631 | 62.937 | 1.00 | 51.41 | F |
| ATOM | 4385 | C5′ | ADE | F | 9 | 120.607 | 109.221 | 60.163 | 1.00 | 53.89 | F |
| ATOM | 4386 | C4′ | ADE | F | 9 | 121.365 | 110.386 | 60.722 | 1.00 | 52.27 | F |
| ATOM | 4387 | O4′ | ADE | F | 9 | 120.391 | 111.447 | 60.753 | 1.00 | 52.46 | F |
| ATOM | 4388 | C1′ | ADE | F | 9 | 119.968 | 111.708 | 62.057 | 1.00 | 50.12 | F |
| ATOM | 4389 | C3′ | ADE | F | 9 | 121.820 | 110.223 | 62.153 | 1.00 | 52.12 | F |
| ATOM | 4390 | O3′ | ADE | F | 9 | 122.868 | 111.161 | 62.349 | 1.00 | 52.41 | F |
| ATOM | 4391 | P | CYT | F | 10 | 123.942 | 110.914 | 63.483 | 1.00 | 52.69 | F |
| ATOM | 4392 | O1P | CYT | F | 10 | 123.575 | 109.653 | 64.251 | 1.00 | 53.41 | F |
| ATOM | 4393 | O2P | CYT | F | 10 | 125.279 | 111.060 | 62.899 | 1.00 | 54.19 | F |
| ATOM | 4394 | O5′ | CYT | F | 10 | 123.782 | 112.187 | 64.404 | 1.00 | 52.56 | F |
| ATOM | 4395 | N1 | CYT | F | 10 | 118.902 | 114.023 | 65.166 | 1.00 | 42.59 | F |
| ATOM | 4396 | C6 | CYT | F | 10 | 118.767 | 112.676 | 65.403 | 1.00 | 42.59 | F |
| ATOM | 4397 | C2 | CYT | F | 10 | 117.812 | 114.805 | 64.974 | 1.00 | 42.45 | F |
| ATOM | 4398 | O2 | CYT | F | 10 | 118.010 | 115.991 | 64.697 | 1.00 | 41.40 | F |
| ATOM | 4399 | N3 | CYT | F | 10 | 116.567 | 114.279 | 65.098 | 1.00 | 43.47 | F |
| ATOM | 4400 | C4 | CYT | F | 10 | 116.421 | 112.985 | 65.395 | 1.00 | 43.84 | F |
| ATOM | 4401 | N4 | CYT | F | 10 | 115.173 | 112.522 | 65.560 | 1.00 | 42.53 | F |
| ATOM | 4402 | C5 | CYT | F | 10 | 117.557 | 112.119 | 65.544 | 1.00 | 42.78 | F |
| ATOM | 4403 | C2′ | CYT | F | 10 | 120.931 | 114.557 | 66.548 | 1.00 | 47.47 | F |
| ATOM | 4404 | C5′ | CYT | F | 10 | 122.540 | 112.521 | 64.911 | 1.00 | 50.04 | F |
| ATOM | 4405 | C4′ | CYT | F | 10 | 122.364 | 113.996 | 64.757 | 1.00 | 47.50 | F |
| ATOM | 4406 | O4′ | CYT | F | 10 | 121.042 | 114.176 | 64.220 | 1.00 | 44.98 | F |
| ATOM | 4407 | C1′ | CYT | F | 10 | 120.202 | 114.709 | 65.209 | 1.00 | 45.11 | F |
| ATOM | 4408 | C3′ | CYT | F | 10 | 122.380 | 114.657 | 66.127 | 1.00 | 48.69 | F |
| ATOM | 4409 | O3′ | CYT | F | 10 | 122.620 | 116.048 | 66.135 | 1.00 | 52.11 | F |
| ATOM | 4410 | O5′ | GUA | G | 1 | 47.014 | 140.673 | 96.657 | 1.00 | 38.92 | G |
| ATOM | 4411 | N9 | GUA | G | 1 | 43.791 | 142.655 | 96.443 | 1.00 | 36.26 | G |
| ATOM | 4412 | C4 | GUA | G | 1 | 42.700 | 143.421 | 96.775 | 1.00 | 35.90 | G |
| ATOM | 4413 | N3 | GUA | G | 1 | 41.433 | 142.985 | 96.862 | 1.00 | 37.97 | G |
| ATOM | 4414 | C2 | GUA | G | 1 | 40.601 | 143.959 | 97.239 | 1.00 | 39.06 | G |
| ATOM | 4415 | N2 | GUA | G | 1 | 39.261 | 143.701 | 97.379 | 1.00 | 37.23 | G |
| ATOM | 4416 | N1 | GUA | G | 1 | 41.019 | 145.245 | 97.523 | 1.00 | 38.08 | G |
| ATOM | 4417 | C6 | GUA | G | 1 | 42.341 | 145.680 | 97.496 | 1.00 | 37.00 | G |
| ATOM | 4418 | O6 | GUA | G | 1 | 42.637 | 146.821 | 97.904 | 1.00 | 36.07 | G |
| ATOM | 4419 | C5 | GUA | G | 1 | 43.201 | 144.675 | 97.042 | 1.00 | 35.62 | G |
| ATOM | 4420 | N7 | GUA | G | 1 | 44.567 | 144.705 | 96.859 | 1.00 | 35.61 | G |
| ATOM | 4421 | C8 | GUA | G | 1 | 44.873 | 143.483 | 96.509 | 1.00 | 36.26 | G |
| ATOM | 4422 | C2′ | GUA | G | 1 | 44.656 | 140.768 | 94.954 | 1.00 | 34.97 | G |
| ATOM | 4423 | C5′ | GUA | G | 1 | 46.434 | 139.614 | 97.380 | 1.00 | 36.88 | G |
| ATOM | 4424 | C4′ | GUA | G | 1 | 45.022 | 139.395 | 96.884 | 1.00 | 38.10 | G |
| ATOM | 4425 | O4′ | GUA | G | 1 | 44.195 | 140.520 | 97.286 | 1.00 | 35.81 | G |
| ATOM | 4426 | C1′ | GUA | G | 1 | 43.789 | 141.241 | 96.121 | 1.00 | 36.37 | G |
| ATOM | 4427 | C3′ | GUA | G | 1 | 44.886 | 139.316 | 95.344 | 1.00 | 35.81 | G |
| ATOM | 4428 | O3′ | GUA | G | 1 | 43.748 | 138.493 | 95.080 | 1.00 | 35.65 | G |
| ATOM | 4429 | P | THY | G | 2 | 43.292 | 138.174 | 93.593 | 1.00 | 36.01 | G |
| ATOM | 4430 | O1P | THY | G | 2 | 44.066 | 138.930 | 92.637 | 1.00 | 31.68 | G |
| ATOM | 4431 | O2P | THY | G | 2 | 43.282 | 136.639 | 93.548 | 1.00 | 38.33 | G |
| ATOM | 4432 | O5′ | THY | G | 2 | 41.752 | 138.567 | 93.574 | 1.00 | 33.00 | G |
| ATOM | 4433 | N1 | THY | G | 2 | 40.151 | 143.199 | 93.514 | 1.00 | 33.65 | G |
| ATOM | 4434 | C6 | THY | G | 2 | 41.510 | 143.199 | 93.312 | 1.00 | 32.12 | G |
| ATOM | 4435 | C2 | THY | G | 2 | 39.485 | 144.337 | 93.844 | 1.00 | 34.36 | G |
| ATOM | 4436 | O2 | THY | G | 2 | 38.270 | 144.367 | 94.004 | 1.00 | 36.43 | G |
| ATOM | 4437 | N3 | THY | G | 2 | 40.269 | 145.438 | 93.984 | 1.00 | 33.28 | G |
| ATOM | 4438 | C4 | THY | G | 2 | 41.629 | 145.511 | 93.847 | 1.00 | 33.39 | G |
| ATOM | 4439 | O4 | THY | G | 2 | 42.224 | 146.571 | 94.092 | 1.00 | 34.54 | G |
| ATOM | 4440 | C5 | THY | G | 2 | 42.270 | 144.300 | 93.454 | 1.00 | 31.47 | G |
| ATOM | 4441 | C5A | THY | G | 2 | 43.742 | 144.340 | 93.230 | 1.00 | 26.26 | G |
| ATOM | 4442 | C2′ | THY | G | 2 | 39.525 | 141.165 | 92.143 | 1.00 | 32.65 | G |
| ATOM | 4443 | C5′ | THY | G | 2 | 41.290 | 139.639 | 94.306 | 1.00 | 32.39 | G |
| ATOM | 4444 | C4′ | THY | G | 2 | 39.806 | 139.824 | 94.103 | 1.00 | 34.01 | G |
| ATOM | 4445 | O4′ | THY | G | 2 | 39.590 | 141.193 | 94.509 | 1.00 | 34.57 | G |
| ATOM | 4446 | C1′ | THY | G | 2 | 39.315 | 142.002 | 93.395 | 1.00 | 33.75 | G |
| ATOM | 4447 | C3′ | THY | G | 2 | 39.297 | 139.758 | 92.654 | 1.00 | 34.40 | G |
| ATOM | 4448 | O3′ | THY | G | 2 | 37.910 | 139.418 | 92.638 | 1.00 | 35.27 | G |
| ATOM | 4449 | P | THY | G | 3 | 37.159 | 139.145 | 91.240 | 1.00 | 36.74 | G |
| ATOM | 4450 | O1P | THY | G | 3 | 38.074 | 139.342 | 90.080 | 1.00 | 36.29 | G |
| ATOM | 4451 | O2P | THY | G | 3 | 36.385 | 137.865 | 91.357 | 1.00 | 38.49 | G |
| ATOM | 4452 | O5′ | THY | G | 3 | 36.105 | 140.320 | 91.188 | 1.00 | 37.18 | G |
| ATOM | 4453 | N1 | THY | G | 3 | 37.521 | 145.189 | 90.681 | 1.00 | 39.81 | G |
| ATOM | 4454 | C6 | THY | G | 3 | 38.638 | 144.512 | 90.222 | 1.00 | 38.98 | G |
| ATOM | 4455 | C2 | THY | G | 3 | 37.615 | 146.436 | 91.219 | 1.00 | 40.10 | G |
| ATOM | 4456 | O2 | THY | G | 3 | 36.636 | 147.035 | 91.666 | 1.00 | 42.50 | G |
| ATOM | 4457 | N3 | THY | G | 3 | 38.892 | 146.966 | 91.234 | 1.00 | 38.89 | G |
| ATOM | 4458 | C4 | THY | G | 3 | 40.058 | 146.366 | 90.796 | 1.00 | 38.22 | G |
| ATOM | 4459 | O4 | THY | G | 3 | 41.138 | 146.963 | 90.897 | 1.00 | 37.01 | G |
| ATOM | 4460 | C5 | THY | G | 3 | 39.888 | 145.025 | 90.252 | 1.00 | 39.31 | G |
| ATOM | 4461 | C5A | THY | G | 3 | 41.101 | 144.285 | 89.770 | 1.00 | 34.18 | G |
| ATOM | 4462 | C2′ | THY | G | 3 | 35.959 | 143.716 | 89.422 | 1.00 | 41.25 | G |
| ATOM | 4463 | C5′ | THY | G | 3 | 36.505 | 141.575 | 91.628 | 1.00 | 38.96 | G |
| ATOM | 4464 | C4′ | THY | G | 3 | 35.399 | 142.564 | 91.426 | 1.00 | 40.55 | G |
| ATOM | 4465 | O4′ | THY | G | 3 | 35.968 | 143.849 | 91.771 | 1.00 | 40.80 | G |
| ATOM | 4466 | C1′ | THY | G | 3 | 36.174 | 144.625 | 90.607 | 1.00 | 40.36 | G |
| ATOM | 4467 | C3′ | THY | G | 3 | 35.002 | 142.670 | 89.961 | 1.00 | 43.41 | G |
| ATOM | 4468 | O3′ | THY | G | 3 | 33.609 | 142.997 | 89.778 | 1.00 | 47.27 | G |
| ATOM | 4469 | P | GUA | G | 4 | 32.991 | 143.182 | 88.272 | 1.00 | 49.65 | G |
| ATOM | 4470 | O1P | GUA | G | 4 | 33.981 | 143.121 | 87.185 | 1.00 | 48.14 | G |
| ATOM | 4471 | O2P | GUA | G | 4 | 31.779 | 142.309 | 88.204 | 1.00 | 50.35 | G |
| ATOM | 4472 | O5′ | GUA | G | 4 | 32.509 | 144.684 | 88.371 | 1.00 | 49.75 | G |
| ATOM | 4473 | N9 | GUA | G | 4 | 34.829 | 147.527 | 88.117 | 1.00 | 44.28 | G |
| ATOM | 4474 | C4 | GUA | G | 4 | 35.916 | 148.354 | 88.350 | 1.00 | 41.44 | G |
| ATOM | 4475 | N3 | GUA | G | 4 | 35.874 | 149.574 | 88.901 | 1.00 | 39.90 | G |
| ATOM | 4476 | C2 | GUA | G | 4 | 37.078 | 150.114 | 88.970 | 1.00 | 41.55 | G |
| ATOM | 4477 | N2 | GUA | G | 4 | 37.257 | 151.336 | 89.494 | 1.00 | 42.60 | G |
| ATOM | 4478 | N1 | GUA | G | 4 | 38.223 | 149.510 | 88.539 | 1.00 | 40.96 | G |
| ATOM | 4479 | C6 | GUA | G | 4 | 38.301 | 148.251 | 87.989 | 1.00 | 41.22 | G |
| ATOM | 4480 | O6 | GUA | G | 4 | 39.408 | 147.790 | 87.671 | 1.00 | 40.92 | G |
| ATOM | 4481 | C5 | GUA | G | 4 | 37.013 | 147.656 | 87.889 | 1.00 | 41.17 | G |
| ATOM | 4482 | N7 | GUA | G | 4 | 36.633 | 146.421 | 87.385 | 1.00 | 42.09 | G |
| ATOM | 4483 | C8 | GUA | G | 4 | 35.332 | 146.379 | 87.557 | 1.00 | 43.12 | G |
| ATOM | 4484 | C2′ | GUA | G | 4 | 32.403 | 147.333 | 87.371 | 1.00 | 47.33 | G |
| ATOM | 4485 | C5′ | GUA | G | 4 | 31.898 | 145.140 | 89.594 | 1.00 | 49.50 | G |
| ATOM | 4486 | C4′ | GUA | G | 4 | 31.744 | 146.642 | 89.560 | 1.00 | 49.41 | G |
| ATOM | 4487 | O4′ | GUA | G | 4 | 33.068 | 147.259 | 89.643 | 1.00 | 47.94 | G |
| ATOM | 4488 | C1′ | GUA | G | 4 | 33.425 | 147.838 | 88.390 | 1.00 | 46.17 | G |
| ATOM | 4489 | C3′ | GUA | G | 4 | 31.155 | 147.105 | 88.220 | 1.00 | 49.22 | G |
| ATOM | 4490 | O3′ | GUA | G | 4 | 30.314 | 148.257 | 88.450 | 1.00 | 50.89 | G |
| ATOM | 4491 | P | CYT | G | 5 | 29.417 | 148.890 | 87.252 | 1.00 | 52.80 | G |
| ATOM | 4492 | O1P | CYT | G | 5 | 29.370 | 147.917 | 86.121 | 1.00 | 52.24 | G |
| ATOM | 4493 | 02P | CYT | G | 5 | 28.183 | 149.310 | 87.904 | 1.00 | 50.60 | G |
| ATOM | 4494 | O5′ | CYT | G | 5 | 30.247 | 150.195 | 86.811 | 1.00 | 50.08 | G |
| ATOM | 4495 | N1 | CYT | G | 5 | 34.543 | 150.810 | 85.451 | 1.00 | 44.66 | G |
| ATOM | 4496 | C6 | CYT | G | 5 | 34.330 | 149.563 | 84.927 | 1.00 | 43.66 | G |
| ATOM | 4497 | C2 | CYT | G | 5 | 35.866 | 151.252 | 85.702 | 1.00 | 44.12 | G |
| ATOM | 4498 | O2 | CYT | G | 5 | 36.041 | 152.359 | 86.230 | 1.00 | 45.32 | G |
| ATOM | 4499 | N3 | CYT | G | 5 | 36.909 | 150.449 | 85.376 | 1.00 | 41.86 | G |
| ATOM | 4500 | C4 | CYT | G | 5 | 36.677 | 149.237 | 84.865 | 1.00 | 40.97 | G |
| ATOM | 4501 | N4 | CYT | G | 5 | 37.715 | 148.469 | 84.588 | 1.00 | 40.81 | G |
| ATOM | 4502 | C5 | CYT | G | 5 | 35.362 | 148.752 | 84.625 | 1.00 | 40.78 | G |
| ATOM | 4503 | C2′ | CYT | G | 5 | 32.112 | 151.743 | 84.956 | 1.00 | 48.83 | G |
| ATOM | 4504 | C5′ | CYT | G | 5 | 30.820 | 150.982 | 87.822 | 1.00 | 48.65 | G |
| ATOM | 4505 | C4′ | CYT | G | 5 | 31.685 | 152.092 | 87.279 | 1.00 | 49.16 | G |
| ATOM | 4506 | O4′ | CYT | G | 5 | 33.075 | 151.661 | 87.113 | 1.00 | 48.10 | G |
| ATOM | 4507 | C1′ | CYT | G | 5 | 33.422 | 151.732 | 85.742 | 1.00 | 46.81 | G |
| ATOM | 4508 | C3′ | CYT | G | 5 | 31.243 | 152.577 | 85.898 | 1.00 | 49.90 | G |
| ATOM | 4509 | O3′ | CYT | G | 5 | 31.498 | 154.000 | 85.807 | 1.00 | 52.88 | G |
| ATOM | 4510 | P | GUA | G | 6 | 30.775 | 154.889 | 84.660 | 1.00 | 56.56 | G |
| ATOM | 4511 | O1P | GUA | G | 6 | 31.155 | 154.352 | 83.295 | 1.00 | 54.54 | G |
| ATOM | 4512 | O2P | GUA | G | 6 | 29.342 | 154.991 | 85.052 | 1.00 | 52.49 | G |
| ATOM | 4513 | O5′ | GUA | G | 6 | 31.508 | 156.300 | 84.746 | 1.00 | 53.05 | G |
| ATOM | 4514 | N9 | GUA | G | 6 | 35.380 | 155.480 | 83.798 | 1.00 | 51.78 | G |
| ATOM | 4515 | C4 | GUA | G | 6 | 36.677 | 155.067 | 83.628 | 1.00 | 50.48 | G |
| ATOM | 4516 | N3 | GUA | G | 6 | 37.782 | 155.795 | 83.875 | 1.00 | 50.29 | G |
| ATOM | 4517 | C2 | GUA | G | 6 | 38.889 | 155.120 | 83.594 | 1.00 | 51.45 | G |
| ATOM | 4518 | N2 | GUA | G | 6 | 40.084 | 155.684 | 83.778 | 1.00 | 51.38 | G |
| ATOM | 4519 | N1 | GUA | G | 6 | 38.906 | 153.834 | 83.105 | 1.00 | 51.72 | G |
| ATOM | 4520 | C6 | GUA | G | 6 | 37.767 | 153.074 | 82.845 | 1.00 | 53.06 | G |
| ATOM | 4521 | O6 | GUA | G | 6 | 37.870 | 151.916 | 82.396 | 1.00 | 55.74 | G |
| ATOM | 4522 | C5 | GUA | G | 6 | 36.588 | 153.780 | 83.149 | 1.00 | 51.55 | G |
| ATOM | 4523 | N7 | GUA | G | 6 | 35.266 | 153.373 | 83.052 | 1.00 | 51.49 | G |
| ATOM | 4524 | C8 | GUA | G | 6 | 34.583 | 154.404 | 83.459 | 1.00 | 51.71 | G |
| ATOM | 4525 | C2′ | GUA | G | 6 | 33.792 | 157.419 | 83.388 | 1.00 | 52.90 | G |
| ATOM | 4526 | C5′ | GUA | G | 6 | 32.048 | 156.747 | 85.961 | 1.00 | 51.85 | G |
| ATOM | 4527 | C4′ | GUA | G | 6 | 33.273 | 157.586 | 85.705 | 1.00 | 52.84 | G |
| ATOM | 4528 | O4′ | GUA | G | 6 | 34.453 | 156.753 | 85.548 | 1.00 | 53.84 | G |
| ATOM | 4529 | C1′ | GUA | G | 6 | 34.925 | 156.810 | 84.204 | 1.00 | 52.43 | G |
| ATOM | 4530 | C3′ | GUA | G | 6 | 33.182 | 158.375 | 84.399 | 1.00 | 53.40 | G |
| ATOM | 4531 | O3′ | GUA | G | 6 | 34.029 | 159.518 | 84.530 | 1.00 | 53.50 | G |
| ATOM | 4532 | P | GUA | G | 7 | 34.007 | 160.651 | 83.426 | 1.00 | 53.54 | G |
| ATOM | 4533 | O1P | GUA | G | 7 | 33.750 | 160.088 | 82.075 | 1.00 | 53.60 | G |
| ATOM | 4534 | O2P | GUA | G | 7 | 33.081 | 161.627 | 84.022 | 1.00 | 54.92 | G |
| ATOM | 4535 | O5′ | GUA | G | 7 | 35.514 | 161.132 | 83.409 | 1.00 | 51.87 | G |
| ATOM | 4536 | N9 | GUA | G | 7 | 37.981 | 157.814 | 80.897 | 1.00 | 48.05 | G |
| ATOM | 4537 | C4 | GUA | G | 7 | 38.753 | 156.766 | 80.461 | 1.00 | 45.94 | G |
| ATOM | 4538 | N3 | GUA | G | 7 | 40.098 | 156.774 | 80.287 | 1.00 | 45.76 | G |
| ATOM | 4539 | C2 | GUA | G | 7 | 40.540 | 155.616 | 79.778 | 1.00 | 44.55 | G |
| ATOM | 4540 | N2 | GUA | G | 7 | 41.840 | 155.427 | 79.532 | 1.00 | 42.99 | G |
| ATOM | 4541 | N1 | GUA | G | 7 | 39.727 | 154.555 | 79.480 | 1.00 | 43.61 | G |
| ATOM | 4542 | C6 | GUA | G | 7 | 38.350 | 154.525 | 79.689 | 1.00 | 44.15 | G |
| ATOM | 4543 | O6 | GUA | G | 7 | 37.701 | 153.499 | 79.425 | 1.00 | 43.57 | G |
| ATOM | 4544 | C5 | GUA | G | 7 | 37.870 | 155.751 | 80.214 | 1.00 | 44.98 | G |
| ATOM | 4545 | N7 | GUA | G | 7 | 36.583 | 156.131 | 80.546 | 1.00 | 45.51 | G |
| ATOM | 4546 | C8 | GUA | G | 7 | 36.705 | 157.353 | 80.970 | 1.00 | 45.98 | G |
| ATOM | 4547 | C2′ | GUA | G | 7 | 37.480 | 160.250 | 80.593 | 1.00 | 53.84 | G |
| ATOM | 4548 | C5′ | GUA | G | 7 | 36.492 | 160.165 | 83.559 | 1.00 | 51.30 | G |
| ATOM | 4549 | C4′ | GUA | G | 7 | 37.775 | 160.592 | 82.920 | 1.00 | 52.18 | G |
| ATOM | 4550 | O4′ | GUA | G | 7 | 38.452 | 159.373 | 82.557 | 1.00 | 53.31 | G |
| ATOM | 4551 | C1′ | GUA | G | 7 | 38.419 | 159.181 | 81.154 | 1.00 | 51.81 | G |
| ATOM | 4552 | C3′ | GUA | G | 7 | 37.611 | 161.361 | 81.620 | 1.00 | 55.14 | G |
| ATOM | 4553 | O3′ | GUA | G | 7 | 38.831 | 162.090 | 81.442 | 1.00 | 56.40 | G |
| ATOM | 4554 | P | THY | G | 8 | 39.019 | 163.019 | 80.185 | 1.00 | 58.64 | G |
| ATOM | 4555 | O1P | THY | G | 8 | 37.688 | 163.061 | 79.474 | 1.00 | 56.59 | G |
| ATOM | 4556 | O2P | THY | G | 8 | 39.701 | 164.274 | 80.646 | 1.00 | 59.83 | G |
| ATOM | 4557 | O5′ | THY | G | 8 | 40.080 | 162.260 | 79.295 | 1.00 | 56.83 | G |
| ATOM | 4558 | N1 | THY | G | 8 | 39.460 | 157.655 | 76.825 | 1.00 | 48.64 | G |
| ATOM | 4559 | C6 | THY | G | 8 | 38.223 | 158.101 | 77.216 | 1.00 | 47.45 | G |
| ATOM | 4560 | C2 | THY | G | 8 | 39.656 | 156.360 | 76.433 | 1.00 | 46.84 | G |
| ATOM | 4561 | O2 | THY | G | 8 | 40.728 | 155.939 | 76.081 | 1.00 | 46.00 | G |
| ATOM | 4562 | N3 | THY | G | 8 | 38.540 | 155.578 | 76.456 | 1.00 | 45.79 | G |
| ATOM | 4563 | C4 | THY | G | 8 | 37.265 | 155.956 | 76.810 | 1.00 | 46.32 | G |
| ATOM | 4564 | O4 | THY | G | 8 | 36.355 | 155.133 | 76.747 | 1.00 | 45.06 | G |
| ATOM | 4565 | C5 | THY | G | 8 | 37.128 | 157.336 | 77.222 | 1.00 | 45.76 | G |
| ATOM | 4566 | C5A | THY | G | 8 | 35.789 | 157.854 | 77.623 | 1.00 | 43.68 | G |
| ATOM | 4567 | C2′ | THY | G | 8 | 40.462 | 159.857 | 75.980 | 1.00 | 53.92 | G |
| ATOM | 4568 | C5′ | THY | G | 8 | 39.803 | 160.969 | 78.849 | 1.00 | 55.70 | G |
| ATOM | 4569 | C4′ | THY | G | 8 | 41.035 | 160.392 | 78.221 | 1.00 | 55.10 | G |
| ATOM | 4570 | O4′ | THY | G | 8 | 40.833 | 158.969 | 78.145 | 1.00 | 54.29 | G |
| ATOM | 4571 | C1′ | THY | G | 8 | 40.614 | 158.568 | 76.805 | 1.00 | 52.28 | G |
| ATOM | 4572 | C3′ | THY | G | 8 | 41.264 | 160.863 | 76.793 | 1.00 | 55.20 | G |
| ATOM | 4573 | O3′ | THY | G | 8 | 42.661 | 160.795 | 76.557 | 1.00 | 56.45 | G |
| ATOM | 4574 | P | THY | G | 9 | 43.284 | 161.490 | 75.265 | 1.00 | 59.63 | G |
| ATOM | 4575 | O1P | THY | G | 9 | 42.131 | 162.114 | 74.529 | 1.00 | 59.23 | G |
| ATOM | 4576 | O2P | THY | G | 9 | 44.477 | 162.315 | 75.638 | 1.00 | 58.57 | G |
| ATOM | 4577 | O5′ | THY | G | 9 | 43.838 | 160.247 | 74.443 | 1.00 | 58.81 | G |
| ATOM | 4578 | N1 | THY | G | 9 | 40.860 | 156.915 | 72.574 | 1.00 | 49.44 | G |
| ATOM | 4579 | C6 | THY | G | 9 | 40.089 | 157.984 | 72.937 | 1.00 | 48.39 | G |
| ATOM | 4580 | C2 | THY | G | 9 | 40.305 | 155.674 | 72.422 | 1.00 | 48.89 | G |
| ATOM | 4581 | O2 | THY | G | 9 | 40.951 | 154.708 | 72.070 | 1.00 | 48.38 | G |
| ATOM | 4582 | N3 | THY | G | 9 | 38.955 | 155.599 | 72.682 | 1.00 | 47.82 | G |
| ATOM | 4583 | C4 | THY | G | 9 | 38.118 | 156.630 | 73.048 | 1.00 | 47.53 | G |
| ATOM | 4584 | O4 | THY | G | 9 | 36.920 | 156.414 | 73.279 | 1.00 | 45.88 | G |
| ATOM | 4585 | C5 | THY | G | 9 | 38.758 | 157.910 | 73.148 | 1.00 | 48.02 | G |
| ATOM | 4586 | C5A | THY | G | 9 | 37.913 | 159.104 | 73.467 | 1.00 | 48.52 | G |
| ATOM | 4587 | C2′ | THY | G | 9 | 42.790 | 158.336 | 71.696 | 1.00 | 55.69 | G |
| ATOM | 4588 | C5′ | THY | G | 9 | 43.370 | 158.944 | 74.722 | 1.00 | 57.31 | G |
| ATOM | 4589 | C4′ | THY | G | 9 | 43.954 | 157.965 | 73.740 | 1.00 | 56.21 | G |
| ATOM | 4590 | O4′ | THY | G | 9 | 42.972 | 156.929 | 73.572 | 1.00 | 54.76 | G |
| ATOM | 4591 | C1′ | THY | G | 9 | 42.313 | 157.036 | 72.324 | 1.00 | 52.67 | G |
| ATOM | 4592 | C3′ | THY | G | 9 | 44.157 | 158.540 | 72.346 | 1.00 | 57.79 | G |
| ATOM | 4593 | O3′ | THY | G | 9 | 45.263 | 157.822 | 71.753 | 1.00 | 59.82 | G |
| ATOM | 4594 | P | GUA | G | 10 | 45.807 | 158.233 | 70.307 | 1.00 | 62.80 | G |
| ATOM | 4595 | O1P | GUA | G | 10 | 45.018 | 159.474 | 69.818 | 1.00 | 63.72 | G |
| ATOM | 4596 | O2P | GUA | G | 10 | 47.286 | 158.245 | 70.310 | 1.00 | 62.06 | G |
| ATOM | 4597 | O5′ | GUA | G | 10 | 45.372 | 157.009 | 69.365 | 1.00 | 63.52 | G |
| ATOM | 4598 | N9 | GUA | G | 10 | 42.042 | 155.441 | 68.236 | 1.00 | 50.25 | G |
| ATOM | 4599 | C4 | GUA | G | 10 | 40.828 | 154.816 | 68.399 | 1.00 | 47.09 | G |
| ATOM | 4600 | N3 | GUA | G | 10 | 40.525 | 153.557 | 68.008 | 1.00 | 43.77 | G |
| ATOM | 4601 | C2 | GUA | G | 10 | 39.255 | 153.265 | 68.280 | 1.00 | 45.16 | G |
| ATOM | 4602 | N2 | GUA | G | 10 | 38.729 | 152.031 | 67.956 | 1.00 | 44.71 | G |
| ATOM | 4603 | N1 | GUA | G | 10 | 38.380 | 154.150 | 68.895 | 1.00 | 45.81 | G |
| ATOM | 4604 | C6 | GUA | G | 10 | 38.694 | 155.440 | 69.308 | 1.00 | 45.86 | G |
| ATOM | 4605 | O6 | GUA | G | 10 | 37.831 | 156.152 | 69.861 | 1.00 | 47.27 | G |
| ATOM | 4606 | C5 | GUA | G | 10 | 40.023 | 155.755 | 69.019 | 1.00 | 45.96 | G |
| ATOM | 4607 | N7 | GUA | G | 10 | 40.727 | 156.927 | 69.253 | 1.00 | 48.27 | G |
| ATOM | 4608 | C8 | GUA | G | 10 | 41.917 | 156.699 | 68.776 | 1.00 | 48.57 | G |
| ATOM | 4609 | C2′ | GUA | G | 10 | 44.091 | 155.982 | 66.960 | 1.00 | 57.06 | G |
| ATOM | 4610 | C5′ | GUA | G | 10 | 45.800 | 155.667 | 69.636 | 1.00 | 60.57 | G |
| ATOM | 4611 | C4′ | GUA | G | 10 | 45.413 | 154.737 | 68.497 | 1.00 | 59.27 | G |
| ATOM | 4612 | O4′ | GUA | G | 10 | 44.032 | 154.288 | 68.619 | 1.00 | 56.96 | G |
| ATOM | 4613 | C1′ | GUA | G | 10 | 43.229 | 154.894 | 67.606 | 1.00 | 54.05 | G |
| ATOM | 4614 | C3′ | GUA | G | 10 | 45.489 | 155.405 | 67.121 | 1.00 | 58.50 | G |
| ATOM | 4615 | O3′ | GUA | G | 10 | 45.659 | 154.372 | 66.139 | 1.00 | 60.36 | G |
| ATOM | 4616 | O5′ | CYT | H | 1 | 30.838 | 150.754 | 70.169 | 1.00 | 50.90 | H |
| ATOM | 4617 | N1 | CYT | H | 1 | 34.211 | 151.390 | 69.707 | 1.00 | 43.12 | H |
| ATOM | 4618 | C6 | CYT | H | 1 | 33.407 | 152.216 | 70.457 | 1.00 | 42.63 | H |
| ATOM | 4619 | C2 | CYT | H | 1 | 35.464 | 151.848 | 69.232 | 1.00 | 40.48 | H |
| ATOM | 4620 | O2 | CYT | H | 1 | 36.185 | 151.084 | 68.606 | 1.00 | 37.31 | H |
| ATOM | 4621 | N3 | CYT | H | 1 | 35.845 | 153.109 | 69.481 | 1.00 | 39.15 | H |
| ATOM | 4622 | C4 | CYT | H | 1 | 35.056 | 153.917 | 70.176 | 1.00 | 41.39 | H |
| ATOM | 4623 | N4 | CYT | H | 1 | 35.493 | 155.166 | 70.363 | 1.00 | 40.04 | H |
| ATOM | 4624 | C5 | CYT | H | 1 | 33.778 | 153.475 | 70.711 | 1.00 | 42.21 | H |
| ATOM | 4625 | C2′ | CYT | H | 1 | 33.119 | 149.210 | 70.498 | 1.00 | 46.37 | H |
| ATOM | 4626 | C5′ | CYT | H | 1 | 30.491 | 150.053 | 68.963 | 1.00 | 48.89 | H |
| ATOM | 4627 | C4′ | CYT | H | 1 | 31.697 | 149.229 | 68.585 | 1.00 | 48.01 | H |
| ATOM | 4628 | O4′ | CYT | H | 1 | 32.821 | 150.116 | 68.315 | 1.00 | 46.83 | H |
| ATOM | 4629 | C1′ | CYT | H | 1 | 33.781 | 150.020 | 69.368 | 1.00 | 45.82 | H |
| ATOM | 4630 | C3′ | CYT | H | 1 | 32.167 | 148.317 | 69.715 | 1.00 | 46.40 | H |
| ATOM | 4631 | O3′ | CYT | H | 1 | 32.861 | 147.227 | 69.107 | 1.00 | 44.93 | H |
| ATOM | 4632 | P | ADE | H | 2 | 33.156 | 145.897 | 69.942 | 1.00 | 45.50 | H |
| ATOM | 4633 | O1P | ADE | H | 2 | 32.223 | 145.857 | 71.122 | 1.00 | 45.82 | H |
| ATOM | 4634 | O2P | ADE | H | 2 | 33.186 | 144.778 | 68.972 | 1.00 | 43.98 | H |
| ATOM | 4635 | O5′ | ADE | H | 2 | 34.637 | 146.139 | 70.467 | 1.00 | 42.98 | H |
| ATOM | 4636 | N9 | ADE | H | 2 | 36.531 | 149.174 | 72.494 | 1.00 | 42.33 | H |
| ATOM | 4637 | C4 | ADE | H | 2 | 37.096 | 150.422 | 72.366 | 1.00 | 41.46 | H |
| ATOM | 4638 | N3 | ADE | H | 2 | 38.282 | 150.724 | 71.807 | 1.00 | 42.57 | H |
| ATOM | 4639 | C2 | ADE | H | 2 | 38.505 | 152.045 | 71.862 | 1.00 | 42.87 | H |
| ATOM | 4640 | N1 | ADE | H | 2 | 37.726 | 153.018 | 72.376 | 1.00 | 40.76 | H |
| ATOM | 4641 | C6 | ADE | H | 2 | 36.532 | 152.668 | 72.916 | 1.00 | 40.83 | H |
| ATOM | 4642 | N6 | ADE | H | 2 | 35.742 | 153.626 | 73.413 | 1.00 | 38.22 | H |
| ATOM | 4643 | C5 | ADE | H | 2 | 36.189 | 151.301 | 72.919 | 1.00 | 41.39 | H |
| ATOM | 4644 | N7 | ADE | H | 2 | 35.071 | 150.618 | 73.394 | 1.00 | 42.77 | H |
| ATOM | 4645 | C8 | ADE | H | 2 | 35.325 | 149.366 | 73.121 | 1.00 | 42.15 | H |
| ATOM | 4646 | C2′ | ADE | H | 2 | 36.694 | 146.571 | 72.571 | 1.00 | 40.65 | H |
| ATOM | 4647 | C5′ | ADE | H | 2 | 35.665 | 146.167 | 69.543 | 1.00 | 40.30 | H |
| ATOM | 4648 | C4′ | ADE | H | 2 | 36.988 | 146.473 | 70.193 | 1.00 | 41.50 | H |
| ATOM | 4649 | O4′ | ADE | H | 2 | 37.054 | 147.861 | 70.613 | 1.00 | 41.80 | H |
| ATOM | 4650 | C1′ | ADE | H | 2 | 37.150 | 147.928 | 72.032 | 1.00 | 42.62 | H |
| ATOM | 4651 | C3′ | ADE | H | 2 | 37.207 | 145.659 | 71.469 | 1.00 | 41.16 | H |
| ATOM | 4652 | O3′ | ADE | H | 2 | 38.591 | 145.366 | 71.555 | 1.00 | 40.03 | H |
| ATOM | 4653 | P | ADE | H | 3 | 39.072 | 143.972 | 72.169 | 1.00 | 40.26 | H |
| ATOM | 4654 | O1P | ADE | H | 3 | 38.106 | 143.672 | 73.252 | 1.00 | 39.24 | H |
| ATOM | 4655 | O2P | ADE | H | 3 | 39.257 | 142.973 | 71.110 | 1.00 | 40.42 | H |
| ATOM | 4656 | O5′ | ADE | H | 3 | 40.483 | 144.442 | 72.757 | 1.00 | 37.80 | H |
| ATOM | 4657 | N9 | ADE | H | 3 | 40.060 | 149.362 | 74.337 | 1.00 | 36.11 | H |
| ATOM | 4658 | C4 | ADE | H | 3 | 39.798 | 150.659 | 74.695 | 1.00 | 33.24 | H |
| ATOM | 4659 | N3 | ADE | H | 3 | 40.555 | 151.720 | 74.440 | 1.00 | 33.72 | H |
| ATOM | 4660 | C2 | ADE | H | 3 | 40.059 | 152.777 | 74.990 | 1.00 | 32.42 | H |
| ATOM | 4661 | N1 | ADE | H | 3 | 38.952 | 152.913 | 75.692 | 1.00 | 33.15 | H |
| ATOM | 4662 | C6 | ADE | H | 3 | 38.181 | 151.848 | 75.909 | 1.00 | 32.47 | H |
| ATOM | 4663 | N6 | ADE | H | 3 | 37.013 | 152.032 | 76.560 | 1.00 | 30.84 | H |
| ATOM | 4664 | C5 | ADE | H | 3 | 38.635 | 150.629 | 75.414 | 1.00 | 33.01 | H |
| ATOM | 4665 | N7 | ADE | H | 3 | 38.141 | 149.330 | 75.499 | 1.00 | 33.43 | H |
| ATOM | 4666 | C8 | ADE | H | 3 | 39.006 | 148.623 | 74.818 | 1.00 | 34.68 | H |
| ATOM | 4667 | C2′ | ADE | H | 3 | 42.025 | 148.230 | 74.848 | 1.00 | 40.11 | H |
| ATOM | 4668 | C5′ | ADE | H | 3 | 40.525 | 145.611 | 73.606 | 1.00 | 39.61 | H |
| ATOM | 4669 | C4′ | ADE | H | 3 | 41.596 | 146.553 | 73.124 | 1.00 | 38.73 | H |
| ATOM | 4670 | O4′ | ADE | H | 3 | 40.938 | 147.819 | 72.793 | 1.00 | 37.40 | H |
| ATOM | 4671 | C1′ | ADE | H | 3 | 41.286 | 148.873 | 73.698 | 1.00 | 37.27 | H |
| ATOM | 4672 | C3′ | ADE | H | 3 | 42.506 | 146.925 | 74.277 | 1.00 | 38.60 | H |
| ATOM | 4673 | O3′ | ADE | H | 3 | 43.811 | 146.483 | 74.414 | 1.00 | 36.13 | H |
| ATOM | 4674 | P | CYT | H | 4 | 44.324 | 146.172 | 75.874 | 1.00 | 36.55 | H |
| ATOM | 4675 | O1P | CYT | H | 4 | 43.177 | 145.752 | 76.710 | 1.00 | 32.41 | H |
| ATOM | 4676 | O2P | CYT | H | 4 | 45.495 | 145.253 | 75.628 | 1.00 | 36.43 | H |
| ATOM | 4677 | O5′ | CYT | H | 4 | 44.826 | 147.588 | 76.439 | 1.00 | 35.22 | H |
| ATOM | 4678 | N1 | CYT | H | 4 | 42.846 | 151.103 | 77.637 | 1.00 | 39.47 | H |
| ATOM | 4679 | C6 | CYT | H | 4 | 42.143 | 149.938 | 77.608 | 1.00 | 39.62 | H |
| ATOM | 4680 | C2 | CYT | H | 4 | 42.193 | 152.301 | 77.918 | 1.00 | 39.27 | H |
| ATOM | 4681 | O2 | CYT | H | 4 | 42.801 | 153.355 | 77.771 | 1.00 | 41.72 | H |
| ATOM | 4682 | N3 | CYT | H | 4 | 40.911 | 152.274 | 78.324 | 1.00 | 36.42 | H |
| ATOM | 4683 | C4 | CYT | H | 4 | 40.268 | 151.114 | 78.401 | 1.00 | 35.75 | H |
| ATOM | 4684 | N4 | CYT | H | 4 | 39.052 | 151.108 | 78.882 | 1.00 | 33.28 | H |
| ATOM | 4685 | C5 | CYT | H | 4 | 40.862 | 149.899 | 78.000 | 1.00 | 37.72 | H |
| ATOM | 4686 | C2′ | CYT | H | 4 | 44.964 | 150.023 | 78.380 | 1.00 | 43.66 | H |
| ATOM | 4687 | C5′ | CYT | H | 4 | 45.303 | 148.534 | 75.519 | 1.00 | 38.49 | H |
| ATOM | 4688 | C4′ | CYT | H | 4 | 45.761 | 149.829 | 76.147 | 1.00 | 40.81 | H |
| ATOM | 4689 | O4′ | CYT | H | 4 | 44.632 | 150.760 | 76.181 | 1.00 | 39.08 | H |
| ATOM | 4690 | C1′ | CYT | H | 4 | 44.302 | 151.090 | 77.497 | 1.00 | 39.36 | H |
| ATOM | 4691 | C3′ | CYT | H | 4 | 46.207 | 149.675 | 77.594 | 1.00 | 42.92 | H |
| ATOM | 4692 | O3′ | CYT | H | 4 | 47.286 | 150.493 | 77.994 | 1.00 | 46.72 | H |
| ATOM | 4693 | P | CYT | H | 5 | 47.896 | 150.273 | 79.466 | 1.00 | 50.72 | H |
| ATOM | 4694 | O1P | CYT | H | 5 | 47.321 | 149.067 | 80.109 | 1.00 | 49.00 | H |
| ATOM | 4695 | O2P | CYT | H | 5 | 49.370 | 150.416 | 79.293 | 1.00 | 50.48 | H |
| ATOM | 4696 | O5′ | CYT | H | 5 | 47.324 | 151.489 | 80.305 | 1.00 | 49.22 | H |
| ATOM | 4697 | N1 | CYT | H | 5 | 43.765 | 152.663 | 81.985 | 1.00 | 42.82 | H |
| ATOM | 4698 | C6 | CYT | H | 5 | 43.777 | 151.363 | 81.578 | 1.00 | 41.70 | H |
| ATOM | 4699 | C2 | CYT | H | 5 | 42.557 | 153.273 | 82.355 | 1.00 | 41.27 | H |
| ATOM | 4700 | O2 | CYT | H | 5 | 42.565 | 154.453 | 82.714 | 1.00 | 43.31 | H |
| ATOM | 4701 | N3 | CYT | H | 5 | 41.406 | 152.551 | 82.317 | 1.00 | 38.27 | H |
| ATOM | 4702 | C4 | CYT | H | 5 | 41.438 | 151.275 | 81.924 | 1.00 | 35.81 | H |
| ATOM | 4703 | N4 | CYT | H | 5 | 40.303 | 150.605 | 81.872 | 1.00 | 32.66 | H |
| ATOM | 4704 | C5 | CYT | H | 5 | 42.643 | 150.636 | 81.552 | 1.00 | 37.91 | H |
| ATOM | 4705 | C2′ | CYT | H | 5 | 46.230 | 152.791 | 82.740 | 1.00 | 48.03 | H |
| ATOM | 4706 | C5′ | CYT | H | 5 | 47.308 | 152.744 | 79.711 | 1.00 | 49.08 | H |
| ATOM | 4707 | C4′ | CYT | H | 5 | 46.844 | 153.806 | 80.669 | 1.00 | 48.96 | H |
| ATOM | 4708 | O4′ | CYT | H | 5 | 45.386 | 153.764 | 80.755 | 1.00 | 46.96 | H |
| ATOM | 4709 | C1′ | CYT | H | 5 | 45.004 | 153.426 | 82.068 | 1.00 | 45.01 | H |
| ATOM | 4710 | C3′ | CYT | H | 5 | 47.363 | 153.575 | 82.095 | 1.00 | 49.34 | H |
| ATOM | 4711 | O3′ | CYT | H | 5 | 47.648 | 154.824 | 82.744 | 1.00 | 52.38 | H |
| ATOM | 4712 | P | GUA | H | 6 | 48.660 | 154.882 | 83.991 | 1.00 | 54.69 | H |
| ATOM | 4713 | O1P | GUA | H | 6 | 49.174 | 153.539 | 84.307 | 1.00 | 54.14 | H |
| ATOM | 4714 | O2P | GUA | H | 6 | 49.622 | 156.004 | 83.756 | 1.00 | 57.09 | H |
| ATOM | 4715 | O5′ | GUA | H | 6 | 47.661 | 155.267 | 85.136 | 1.00 | 52.45 | H |
| ATOM | 4716 | N9 | GUA | H | 6 | 43.131 | 153.056 | 86.180 | 1.00 | 46.25 | H |
| ATOM | 4717 | C4 | GUA | H | 6 | 41.811 | 152.704 | 86.097 | 1.00 | 42.21 | H |
| ATOM | 4718 | N3 | GUA | H | 6 | 40.779 | 153.484 | 86.421 | 1.00 | 41.15 | H |
| ATOM | 4719 | C2 | GUA | H | 6 | 39.624 | 152.891 | 86.198 | 1.00 | 41.59 | H |
| ATOM | 4720 | N2 | GUA | H | 6 | 38.471 | 153.563 | 86.402 | 1.00 | 41.51 | H |
| ATOM | 4721 | N1 | GUA | H | 6 | 39.501 | 151.604 | 85.738 | 1.00 | 41.04 | H |
| ATOM | 4722 | C6 | GUA | H | 6 | 40.560 | 150.772 | 85.434 | 1.00 | 40.66 | H |
| ATOM | 4723 | O6 | GUA | H | 6 | 40.339 | 149.621 | 85.066 | 1.00 | 40.85 | H |
| ATOM | 4724 | C5 | GUA | H | 6 | 41.798 | 151.417 | 85.618 | 1.00 | 40.89 | H |
| ATOM | 4725 | N7 | GUA | H | 6 | 43.086 | 150.971 | 85.370 | 1.00 | 42.28 | H |
| ATOM | 4726 | C8 | GUA | H | 6 | 43.850 | 151.975 | 85.712 | 1.00 | 44.80 | H |
| ATOM | 4727 | C2′ | GUA | H | 6 | 44.840 | 154.126 | 87.666 | 1.00 | 52.12 | H |
| ATOM | 4728 | C5′ | GUA | H | 6 | 46.379 | 154.788 | 85.033 | 1.00 | 52.63 | H |
| ATOM | 4729 | C4′ | GUA | H | 6 | 45.472 | 155.605 | 85.893 | 1.00 | 52.80 | H |
| ATOM | 4730 | O4′ | GUA | H | 6 | 44.147 | 155.086 | 85.610 | 1.00 | 52.86 | H |
| ATOM | 4731 | C1′ | GUA | H | 6 | 43.658 | 154.318 | 86.710 | 1.00 | 49.80 | H |
| ATOM | 4732 | C3′ | GUA | H | 6 | 45.739 | 155.316 | 87.367 | 1.00 | 53.74 | H |
| ATOM | 4733 | O3′ | GUA | H | 6 | 45.527 | 156.456 | 88.199 | 1.00 | 55.02 | H |
| ATOM | 4734 | P | CYT | H | 7 | 45.976 | 156.410 | 89.750 | 1.00 | 56.15 | H |
| ATOM | 4735 | O1P | CYT | H | 7 | 47.164 | 155.509 | 89.916 | 1.00 | 52.89 | H |
| ATOM | 4736 | O2P | CYT | H | 7 | 46.072 | 157.868 | 90.077 | 1.00 | 55.88 | H |
| ATOM | 4737 | O5′ | CYT | H | 7 | 44.662 | 155.883 | 90.457 | 1.00 | 54.75 | H |
| ATOM | 4738 | N1 | CYT | H | 7 | 41.927 | 152.516 | 90.184 | 1.00 | 52.10 | H |
| ATOM | 4739 | C6 | CYT | H | 7 | 43.087 | 151.963 | 89.732 | 1.00 | 51.79 | H |
| ATOM | 4740 | C2 | CYT | H | 7 | 40.701 | 151.942 | 89.864 | 1.00 | 52.28 | H |
| ATOM | 4741 | O2 | CYT | H | 7 | 39.650 | 152.452 | 90.308 | 1.00 | 51.13 | H |
| ATOM | 4742 | N3 | CYT | H | 7 | 40.679 | 150.837 | 89.101 | 1.00 | 52.02 | H |
| ATOM | 4743 | C4 | CYT | H | 7 | 41.815 | 150.281 | 88.701 | 1.00 | 52.16 | H |
| ATOM | 4744 | N4 | CYT | H | 7 | 41.733 | 149.141 | 88.009 | 1.00 | 53.30 | H |
| ATOM | 4745 | C5 | CYT | H | 7 | 43.079 | 150.852 | 89.001 | 1.00 | 51.28 | H |
| ATOM | 4746 | C2′ | CYT | H | 7 | 43.067 | 154.023 | 91.969 | 1.00 | 54.62 | H |
| ATOM | 4747 | C5′ | CYT | H | 7 | 43.458 | 156.549 | 90.132 | 1.00 | 54.23 | H |
| ATOM | 4748 | C4′ | CYT | H | 7 | 42.291 | 156.000 | 90.909 | 1.00 | 55.25 | H |
| ATOM | 4749 | O4′ | CYT | H | 7 | 41.726 | 154.860 | 90.219 | 1.00 | 55.63 | H |
| ATOM | 4750 | C1′ | CYT | H | 7 | 41.922 | 153.712 | 91.026 | 1.00 | 54.04 | H |
| ATOM | 4751 | C3′ | CYT | H | 7 | 42.731 | 155.469 | 92.279 | 1.00 | 55.58 | H |
| ATOM | 4752 | O3′ | CYT | H | 7 | 41.595 | 155.493 | 93.171 | 1.00 | 57.10 | H |
| ATOM | 4753 | P | ADE | H | 8 | 41.641 | 156.379 | 94.519 | 1.00 | 56.99 | H |
| ATOM | 4754 | O1P | ADE | H | 8 | 42.356 | 155.598 | 95.583 | 1.00 | 57.46 | H |
| ATOM | 4755 | O2P | ADE | H | 8 | 42.171 | 157.713 | 94.085 | 1.00 | 55.72 | H |
| ATOM | 4756 | O5′ | ADE | H | 8 | 40.119 | 156.457 | 94.959 | 1.00 | 53.31 | H |
| ATOM | 4757 | N9 | ADE | H | 8 | 39.076 | 152.956 | 94.291 | 1.00 | 52.46 | H |
| ATOM | 4758 | C4 | ADE | H | 8 | 38.892 | 151.754 | 93.637 | 1.00 | 50.36 | H |
| ATOM | 4759 | N3 | ADE | H | 8 | 37.742 | 151.215 | 93.211 | 1.00 | 49.41 | H |
| ATOM | 4760 | C2 | ADE | H | 8 | 37.961 | 150.081 | 92.564 | 1.00 | 47.57 | H |
| ATOM | 4761 | N1 | ADE | H | 8 | 39.093 | 149.478 | 92.304 | 1.00 | 45.45 | H |
| ATOM | 4762 | C6 | ADE | H | 8 | 40.231 | 150.035 | 92.727 | 1.00 | 47.13 | H |
| ATOM | 4763 | N6 | ADE | H | 8 | 41.378 | 149.438 | 92.422 | 1.00 | 47.60 | H |
| ATOM | 4764 | C5 | ADE | H | 8 | 40.147 | 151.228 | 93.453 | 1.00 | 49.19 | H |
| ATOM | 4765 | N7 | ADE | H | 8 | 41.111 | 152.040 | 94.036 | 1.00 | 50.32 | H |
| ATOM | 4766 | C8 | ADE | H | 8 | 40.427 | 153.046 | 94.526 | 1.00 | 51.29 | H |
| ATOM | 4767 | C2′ | ADE | H | 8 | 38.099 | 154.690 | 95.951 | 1.00 | 55.36 | H |
| ATOM | 4768 | C5′ | ADE | H | 8 | 39.176 | 157.051 | 94.125 | 1.00 | 52.98 | H |
| ATOM | 4769 | C4′ | ADE | H | 8 | 37.883 | 156.285 | 94.184 | 1.00 | 54.04 | H |
| ATOM | 4770 | O4′ | ADE | H | 8 | 38.089 | 154.972 | 93.596 | 1.00 | 54.56 | H |
| ATOM | 4771 | C1′ | ADE | H | 8 | 38.034 | 153.952 | 94.600 | 1.00 | 54.46 | H |
| ATOM | 4772 | C3′ | ADE | H | 8 | 37.430 | 156.020 | 95.619 | 1.00 | 55.04 | H |
| ATOM | 4773 | O3′ | ADE | H | 8 | 36.001 | 155.918 | 95.622 | 1.00 | 55.61 | H |
| ATOM | 4774 | P | ADE | H | 9 | 35.169 | 156.132 | 96.997 | 1.00 | 55.91 | H |
| ATOM | 4775 | O1P | ADE | H | 9 | 36.141 | 156.450 | 98.079 | 1.00 | 55.15 | H |
| ATOM | 4776 | O2P | ADE | H | 9 | 34.023 | 157.027 | 96.711 | 1.00 | 56.86 | H |
| ATOM | 4777 | O5′ | ADE | H | 9 | 34.529 | 154.718 | 97.244 | 1.00 | 54.64 | H |
| ATOM | 4778 | N9 | ADE | H | 9 | 37.088 | 150.536 | 97.143 | 1.00 | 50.46 | H |
| ATOM | 4779 | C4 | ADE | H | 9 | 37.644 | 149.492 | 96.440 | 1.00 | 48.35 | H |
| ATOM | 4780 | N3 | ADE | H | 9 | 36.987 | 148.464 | 95.871 | 1.00 | 48.31 | H |
| ATOM | 4781 | C2 | ADE | H | 9 | 37.837 | 147.631 | 95.315 | 1.00 | 47.31 | H |
| ATOM | 4782 | N1 | ADE | H | 9 | 39.174 | 147.698 | 95.263 | 1.00 | 47.40 | H |
| ATOM | 4783 | C6 | ADE | H | 9 | 39.801 | 148.744 | 95.828 | 1.00 | 46.40 | H |
| ATOM | 4784 | N6 | ADE | H | 9 | 41.133 | 148.794 | 95.751 | 1.00 | 44.72 | H |
| ATOM | 4785 | C5 | ADE | H | 9 | 39.004 | 149.711 | 96.456 | 1.00 | 46.90 | H |
| ATOM | 4786 | N7 | ADE | H | 9 | 39.301 | 150.894 | 97.126 | 1.00 | 46.34 | H |
| ATOM | 4787 | C8 | ADE | H | 9 | 38.133 | 151.350 | 97.496 | 1.00 | 47.68 | H |
| ATOM | 4788 | C2′ | ADE | H | 9 | 35.325 | 151.454 | 98.691 | 1.00 | 53.03 | H |
| ATOM | 4789 | C5′ | ADE | H | 9 | 35.024 | 153.637 | 96.511 | 1.00 | 53.98 | H |
| ATOM | 4790 | C4′ | ADE | H | 9 | 34.230 | 152.404 | 96.818 | 1.00 | 53.90 | H |
| ATOM | 4791 | O4′ | ADE | H | 9 | 35.070 | 151.343 | 96.346 | 1.00 | 54.00 | H |
| ATOM | 4792 | C1′ | ADE | H | 9 | 35.659 | 150.660 | 97.436 | 1.00 | 52.19 | H |
| ATOM | 4793 | C3′ | ADE | H | 9 | 34.033 | 152.149 | 98.308 | 1.00 | 54.32 | H |
| ATOM | 4794 | O3′ | ADE | H | 9 | 32.915 | 151.259 | 98.469 | 1.00 | 56.71 | H |
| ATOM | 4795 | P | CYT | H | 10 | 32.124 | 151.177 | 99.904 | 1.00 | 60.78 | H |
| ATOM | 4796 | O1P | CYT | H | 10 | 32.824 | 152.002 | 100.934 | 1.00 | 57.30 | H |
| ATOM | 4797 | O2P | CYT | H | 10 | 30.651 | 151.306 | 99.637 | 1.00 | 59.10 | H |
| ATOM | 4798 | O5′ | CYT | H | 10 | 32.298 | 149.714 | 100.448 | 1.00 | 57.48 | H |
| ATOM | 4799 | N1 | CYT | H | 10 | 36.957 | 147.412 | 99.455 | 1.00 | 45.39 | H |
| ATOM | 4800 | C6 | CYT | H | 10 | 37.305 | 148.521 | 100.164 | 1.00 | 44.04 | H |
| ATOM | 4801 | C2 | CYT | H | 10 | 37.889 | 146.725 | 98.711 | 1.00 | 44.87 | H |
| ATOM | 4802 | O2 | CYT | H | 10 | 37.490 | 145.768 | 98.011 | 1.00 | 45.52 | H |
| ATOM | 4803 | N3 | CYT | H | 10 | 39.188 | 147.103 | 98.753 | 1.00 | 44.25 | H |
| ATOM | 4804 | C4 | CYT | H | 10 | 39.549 | 148.163 | 99.501 | 1.00 | 44.51 | H |
| ATOM | 4805 | N4 | CYT | H | 10 | 40.866 | 148.510 | 99.547 | 1.00 | 43.04 | H |
| ATOM | 4806 | C5 | CYT | H | 10 | 38.584 | 148.924 | 100.233 | 1.00 | 42.52 | H |
| ATOM | 4807 | C2′ | CYT | H | 10 | 35.140 | 146.628 | 100.937 | 1.00 | 50.05 | H |
| ATOM | 4808 | C5′ | CYT | H | 10 | 33.573 | 149.163 | 100.458 | 1.00 | 54.55 | H |
| ATOM | 4809 | C4′ | CYT | H | 10 | 33.539 | 147.847 | 99.740 | 1.00 | 52.02 | H |
| ATOM | 4810 | O4′ | CYT | H | 10 | 34.745 | 147.797 | 98.939 | 1.00 | 50.94 | H |
| ATOM | 4811 | C1′ | CYT | H | 10 | 35.620 | 146.872 | 99.511 | 1.00 | 48.50 | H |
| ATOM | 4812 | C3′ | CYT | H | 10 | 33.643 | 146.748 | 100.787 | 1.00 | 51.00 | H |
| ATOM | 4813 | O3′ | CYT | H | 10 | 33.204 | 145.464 | 100.370 | 1.00 | 52.99 | H |
| ATOM | 4814 | OH2 | TIP | W | 1 | 39.617 | 149.064 | 90.269 | 1.00 | 134.44 | W |
| ATOM | 4815 | OH2 | TIP | W | 2 | 40.848 | 136.007 | 92.184 | 1.00 | 19.65 | W |
| ATOM | 4816 | OH2 | TIP | W | 3 | 54.302 | 136.960 | 67.952 | 1.00 | 39.11 | W |
| ATOM | 4817 | OH2 | TIP | W | 4 | 55.333 | 140.195 | 70.912 | 1.00 | 32.17 | W |
| ATOM | 4818 | OH2 | TIP | W | 5 | 100.203 | 130.958 | 62.972 | 1.00 | 33.13 | W |
| ATOM | 4819 | OH2 | TIP | W | 6 | 107.803 | 145.514 | 65.931 | 1.00 | 30.60 | W |
| ATOM | 4820 | OH2 | TIP | W | 7 | 110.919 | 133.045 | 60.268 | 1.00 | 31.12 | W |
| ATOM | 4821 | OH2 | TIP | W | 8 | 53.130 | 124.003 | 78.104 | 1.00 | 31.95 | W |
| ATOM | 4822 | OH2 | TIP | W | 9 | 44.156 | 142.753 | 74.401 | 1.00 | 31.27 | W |
| ATOM | 4823 | OH2 | TIP | W | 10 | 60.405 | 134.237 | 62.200 | 1.00 | 39.31 | W |
| ATOM | 4824 | OH2 | TIP | W | 11 | 51.745 | 128.694 | 72.296 | 1.00 | 38.54 | W |
| ATOM | 4825 | OH2 | TIP | W | 12 | 62.736 | 115.056 | 74.630 | 1.00 | 50.68 | W |
| ATOM | 4826 | OH2 | TIP | W | 13 | 98.699 | 151.582 | 50.544 | 1.00 | 19.97 | W |
| ATOM | 4827 | OH2 | TIP | W | 14 | 98.616 | 153.649 | 52.432 | 1.00 | 21.92 | W |
| ATOM | 4828 | OH2 | TIP | W | 15 | 119.674 | 126.064 | 56.472 | 1.00 | 36.17 | W |
| ATOM | 4829 | OH2 | TIP | W | 16 | 95.528 | 148.095 | 64.391 | 1.00 | 31.03 | W |
| ATOM | 4830 | OH2 | TIP | W | 17 | 103.780 | 136.827 | 42.456 | 1.00 | 56.82 | W |
| ATOM | 4831 | OH2 | TIP | W | 18 | 94.292 | 143.673 | 53.272 | 1.00 | 28.08 | W |
| ATOM | 4832 | OH2 | TIP | W | 20 | 98.023 | 140.680 | 41.772 | 1.00 | 35.12 | W |
| ATOM | 4833 | OH2 | TIP | W | 21 | 86.488 | 128.894 | 51.070 | 1.00 | 46.42 | W |
| ATOM | 4834 | OH2 | TIP | W | 22 | 89.211 | 147.255 | 54.870 | 1.00 | 41.78 | W |
| ATOM | 4835 | OH2 | TIP | W | 23 | 44.330 | 106.044 | 79.766 | 1.00 | 36.85 | W |
| ATOM | 4836 | OH2 | TIP | W | 24 | 41.712 | 131.058 | 87.700 | 1.00 | 32.99 | W |
| ATOM | 4837 | OH2 | TIP | W | 25 | 43.327 | 135.668 | 83.474 | 1.00 | 25.58 | W |
| ATOM | 4838 | OH2 | TIP | W | 27 | 97.879 | 133.524 | 53.203 | 1.00 | 29.24 | W |
| ATOM | 4839 | OH2 | TIP | W | 28 | 110.637 | 121.176 | 46.103 | 1.00 | 23.17 | W |
| ATOM | 4840 | OH2 | TIP | W | 29 | 106.302 | 124.932 | 44.876 | 1.00 | 26.66 | W |
| ATOM | 4841 | OH2 | TIP | W | 31 | 40.281 | 145.890 | 77.683 | 1.00 | 36.33 | W |
| ATOM | 4842 | OH2 | TIP | W | 32 | 59.764 | 132.826 | 64.442 | 1.00 | 33.21 | W |
| ATOM | 4843 | OH2 | TIP | W | 33 | 115.020 | 143.659 | 57.244 | 1.00 | 42.04 | W |
| ATOM | 4844 | OH2 | TIP | W | 34 | 98.482 | 126.638 | 51.648 | 1.00 | 30.78 | W |
| ATOM | 4845 | OH2 | TIP | W | 35 | 110.268 | 136.670 | 60.681 | 1.00 | 37.17 | W |
| ATOM | 4846 | OH2 | TIP | W | 38 | 40.116 | 137.158 | 90.024 | 1.00 | 27.73 | W |
| ATOM | 4847 | OH2 | TIP | W | 41 | 97.272 | 141.019 | 55.433 | 1.00 | 32.76 | W |
| ATOM | 4848 | OH2 | TIP | W | 42 | 47.076 | 109.999 | 85.776 | 1.00 | 30.34 | W |
| ATOM | 4849 | OH2 | TIP | W | 43 | 50.853 | 142.184 | 77.588 | 1.00 | 32.45 | W |
| ATOM | 4850 | OH2 | TIP | W | 47 | 50.247 | 115.220 | 70.741 | 1.00 | 33.21 | W |
| ATOM | 4851 | OH2 | TIP | W | 49 | 35.299 | 143.359 | 73.684 | 1.00 | 31.25 | W |
| ATOM | 4852 | OH2 | TIP | W | 50 | 42.283 | 126.733 | 86.732 | 1.00 | 28.76 | W |
| ATOM | 4853 | OH2 | TIP | W | 51 | 50.805 | 112.785 | 72.012 | 1.00 | 34.88 | W |
| ATOM | 4854 | OH2 | TIP | W | 52 | 98.030 | 137.932 | 48.167 | 1.00 | 28.07 | W |
| ATOM | 4855 | OH2 | TIP | W | 53 | 116.102 | 122.833 | 55.158 | 1.00 | 38.77 | W |
| ATOM | 4856 | OH2 | TIP | W | 54 | 100.741 | 160.884 | 54.146 | 1.00 | 32.09 | W |
| ATOM | 4857 | OH2 | TIP | W | 55 | 104.757 | 153.055 | 65.232 | 1.00 | 33.96 | W |
| ATOM | 4858 | OH2 | TIP | W | 59 | 48.955 | 105.729 | 74.962 | 1.00 | 33.05 | W |
| ATOM | 4859 | OH2 | TIP | W | 60 | 108.700 | 129.474 | 55.354 | 1.00 | 41.86 | W |
| ATOM | 4860 | OH2 | TIP | W | 61 | 100.649 | 124.079 | 49.263 | 1.00 | 33.29 | W |
| ATOM | 4861 | OH2 | TIP | W | 62 | 36.519 | 142.155 | 86.129 | 1.00 | 31.05 | W |
| ATOM | 4862 | OH2 | TIP | W | 65 | 47.556 | 133.342 | 93.157 | 1.00 | 37.32 | W |
| ATOM | 4863 | OH2 | TIP | W | 69 | 47.360 | 125.268 | 88.812 | 1.00 | 40.31 | W |
| ATOM | 4864 | OH2 | TIP | W | 70 | 52.770 | 121.496 | 90.204 | 1.00 | 24.57 | W |
| ATOM | 4865 | OH2 | TIP | W | 71 | 47.506 | 135.138 | 65.842 | 1.00 | 27.83 | W |
| ATOM | 4866 | OH2 | TIP | W | 72 | 112.287 | 135.318 | 60.857 | 1.00 | 28.28 | W |
| ATOM | 4867 | OH2 | TIP | W | 73 | 44.958 | 135.169 | 66.350 | 1.00 | 24.19 | W |
| ATOM | 4868 | OH2 | TIP | W | 74 | 55.976 | 121.945 | 74.786 | 1.00 | 28.53 | W |
| ATOM | 4869 | OH2 | TIP | W | 75 | 101.166 | 157.108 | 57.782 | 1.00 | 23.43 | W |
| END | | | | | | | | | | | |

1. A crystal comprising the AML1 Runt domain.

2. A crystal comprising CBFβ.

3. The crystal of claim 1 or 2 comprising a complex between the AML1 Runt domain and CBFβ and optionally the cognate DNA binding site.

4. The crystal claim 3, which comprises six Runt domain subunits and four CBFβ subunits arranged as two dimers of Runt domain/CBFβ heterodimers (Runt domain/CBFβ) 2 and a Runt domain homodimer.

5. A crystal comprising the regions of AML1 Runt domain which are involved in heterodimerisation with CBFβ.

6. The crystal of claim 5, which comprises loops βF-G, βO-A, βB-C and βC-D and strands βC, βF and βG.

7. A crystal comprising the regions of CBFβ which are involved in heterodimerisation with AML1.

8. The crystal of claim 7, which comprises the following elements of secondary structure: amino-terminal loop and helix H1; strand β1 and loop β1-H3; strands β2, β3 and the connecting loop β2-β3; and strand β4 and at least part of loop β4-β5.

9. The crystal of claim 5 or 7, wherein the residues involved in heterodimerisation are as shown in Table 2.

10. The crystal of claim 1, 2, 5, or 7, wherein the crystal has P21 symmetry.

11. The crystal of claim 1, 2, 5, or 7, wherein said crystal comprises a unit cell having the following dimensions: a=103.2 A, b=79.4 A, c=130.1 A.

12. A crystal having the structure co-ordinates as shown in Table 3 or Table 4.

13. A model for at least part of the AML1 Runt domain and/or at least part of CBFβ, made using the crystal of claim 1, 2, 5, 7, or 12.

14. A method of screening for a ligand capable of binding the AML1 Runt domain and/or CBFβ, comprising analyzing the three dimensional structure of said AML1 Runt domain and/or CBFβ subunit by a computer modeling program.

15. A model for DNA binding by the AML1 Runt domain, made using the crystal according to any of claims 1, 2, 5, 7, or 12.

16. A model according to claim 15, made by least squares superimposition of the coordinates of the Stat3β/DNA complex on to the AML1 Runt domain.

17. A method of screening for a ligand capable of modulating the DNA-binding capacity of the AML1 Runt domain, comprising analyzing the three dimensional structure of said AML1 Runt domain by a computer modeling program.

18. A computer readable medium having stored thereon, structure data of a crystal according to any of claims 1, 2, 5, 7, or 12.

19. A computer readable medium having stored thereon, model data for at least part of the AML1 Runt domain and/or at least part of CBFβ according to claim 13.

20. A computer readable medium having stored thereon, model data for DNA binding by the AML1 Runt domain according to claim 15.

21. A method for producing a medicament to treat and/or prevent a disease in a mammal comprising:
(a) analyzing the three dimensional structure of an AML1 Runt domain and/or CBFβ subunit by a computer modeling program so as to identify a ligand which is capable of interacting with said AML1 Runt domain and/or CBFβ subunit;
(b) mixing said ligand with a pharmaceutically acceptable carrier.

22. A method for the treatment and/or prevention of a disease associated with chromosomal translocations involving the AML1 and/or the CBFB gene, comprising administering to an animal in need of said treatment and/or prevention a pharmaceutical formulation comprising a ligand which is capable of interacting with an AML1 Runt domain and/or CBFβ subunit, and a pharmaceutical carrier.

23. The method of claim 22, wherein said disease is selected from the group consisting of acute myeloid leukaemia (AML), childhood acute lymphoblastic leukaemia, therapy-related leukaemia, myelodysplasia, familial platelet disorder (FDP), cleidocranial dysplasia (CCD), and conditions associated with impaired osteogenesis and/or haematopoiesis.
